SEQUENCE LISTING (288 sequences)

SEQ ID NO: 1 - DNA, 202001 bp, Homo sapiens
  source 1..202001: mol_type = genomic DNA
gcccagcaggtgtcagcctcattttaccccgcccctattcaagatgaagttgttctggttccaacgcctctgacatattagctgcatcattttacatttcttttttttttttccttttaaatggggtcttgctctgtcacccaggctggagtgctgtggtatgatctcggctcactgcaatctccacctccgaggttccagcgattctcttgcctcagcctcccgagtagctgggactacaggcacccaccatcatactgggctaatttttgtgtttttagtagagatggggtttccccatgttgcccaggctgatctcaaactcctgggcttaagcaatacagccgcgttggcctcccaaagtgttgggattacaagcatgagctaccccacccagctcattttacatttccacttgttaaactgaaaactggcccgagaaagcttctgtactgccatccttgcgtccttgcagatgaatcgtaacctagcatagtaggtaggcagactgaaaacctaacttagcagtaggcttctgtaacaacagctgtgtctcagccagttcctgcagccagacttcaaccactcacaggccgcaaactgttcaaactgtgttcggagaaggcgaattcatctggctgttaacgtgcctcacttctgctttctgtggccactttcccttttctgtccataaatttgctttgaccacacagcatccctagagtctccctgaatctgctgtgattctgggacctgcaccatttgtgaattgttttttttttccttgatcagctaaactctgttcaattcaatttgttggaagtttttaacataccaatggtgcaccaaggttccaatttctccacttcctcataaataagtcattttaaatggcttttcagtattccaatatttggaagtattaatgtttctaccaattttctatttttggacattgaggttgtttcattttttttttctttttttgagacagagtctcgctccgtcacccaggctggagtgcagtggcctgatcccggcccactgcaacctccacctccctcctcagcctcctgagtagctgggattacaggtgcatgcaccaccacacccagctaatttttgtatttttagtagagatggggtttcaccatgttggtcaggctggtctcaaactcctgacctcaggtggtccacctgccttggcctcccaaaatgctgggattacaggcctgagccactgcgcctggcctcatcttcttgatattaatgttgctttaacatctttgtccctgtgttttttgtttttttttttgagacggagtctcattcattctgtcacccaggctggagttcagtggcgtgatctcagctcactgcaacctctgtctcctgggttccagtgattctcctgcgtcggtctcctgagtagctgtgttcctgggtctttcgatggttatttaatacttccctacagtaatgccctgtgcgtacatgctaagtgtgatgaaatggttggcacagttaaatcttttgaaagacattgccaagtcactcttcagaaaagtgataggaggtcatagcaattttaagaagtcctcatttctacatttccttactaatctcggttggtgtctcttcaatctttcctcacacttttcttgggtttttcctgaatcatgagtctactacatttacacattttaaagcatctttagaaacaggatctcattttgttgcccaggctagagtttggtggcatgattatagctcctcatactcctgggctcaagtgatccttccacctctgaaaccccaaaatttgagaaaggtctcatttaatttagaaagtttattttgccaaggttgagggtgcacacctgtgatgatatacgagttaaaaagaaattatttaggcagatactgagggtaagaaagtcctcggtaaggttttcttttcaatgaaaagcagcccccaagcattttcttttctaacaaagagcagcctgtaaaatcgagctgcagacatacacaagcaagctggaagcttgcacaggtgaatgctggcagctgtgccaataagaaaaggctacctggggccaggcagatccaacatggcggctccatcttccctttccttgtcaaccatgtgcacagtaaggagcaggcaacatagtgtcccccgagtagagaccaatttgcataataaaaggtgagggtagggtgggcagcttctttgcatgctatgtaaacattatgcctggtccaaccaatctttgggccctgtgtaaattagacaccacctcctcaagcctgtctataaaaccctgtccattctgccgcaggctggaagacccactggggcacccctctctctctataggagacagctattcatttttctctttctttcacctattaaagctccactcttaaccccactccgtgtgtatctatgttcttgatttccttggcatgaggcaatgaaccttgggtattaccccagaaccttgggtattatgccacttcagtgacacagcctcaggaaatcctgatgacatgttcccaagatggtcggggcacagcttggttttatacattttagggagacatgagacgtcaattcatatatgtaagaagtacattggttccgtccagaaaggcggggacaacttgaggcagggagagagcttctaggtcacaggtagacaaatggttgcattcttttgaatctccgataagcctttccaaaggaggcaatcagaatatgcgtctattgactgggcgcagtggctcatgcctgtaatgccagcactttgggaggcggaggtgggtggatcacctgaggtcaggagtttgagagcagcccggccaacatggtgaaaccctgtctctactaaaaatacaaaaaattagctgggcgtggtggcgggcgcctgtaatcccagctactcgggaggctgaggcaggagaatagcttgaacccagaaggaagaggttgcagtgagctgagatggtgccattgcactccagcctgggcaacaagagtgaaactccatctcagaaaaaaaaaaaaaaggcctgggcaaagtggctcacgcctgtaatcccagcactttgggaagccgaggcgggcaggtcacaaagtcaggagattgagaccatcctggctaacatgatgaaaccccatctctactaaaaaatacaaaaaactagctgggtgtggtggcgagcacctgtagtcccagctactcggcaggctgaggcaggagaatggcgtgaaccggggaggcggagcttgcagtgagccgagatcacaccactgcactccagcccggacgacagggcaagactctatctcaaattaaaaaaaaaaaaaaaaaaaaaaaaaaagagagagagaatatgcatctatctcagtgagcagaaggatgactttgaatggaatgggagcagttcctagcttgaacttcccctttagcttcagtgatttgggggctcaaggtatgttcctttcacatacctcagcctcccaagtagctgggaccacaagtgcatgccaccacacgtggctaatgttttattttttttgtaggaatagggtctcactatgtgtccaggctggtctaaaacccctgagctcaaatggtcctcccgcctcagcctcccgaaatgctgggattacaggcatgagccagcatgcccggcctagtctacatttttataaattgctaattcaaagttccctctccaaaacctcatggttttccctgttctcatcccctgcaccctcccttcccctggagtactcacctggccttggaggtctggtgtgagcccggacttcgattctaggcacagcatgtgatgagcgcccccaggtcaaacacctcccctctgcggcctgtgcttcaccgccttgacagtgagaaaggtctcccttcggctcattctcgaagtctcaaacttcacttctcctgtgcgctgattctgaattcagcccccgtccaaggtcctggcccctttctcttctgcttggcgtgttgttcatcaccactgtgcactgctgagggtaagtgcggttctctggacctctgctttatcattagaacagactcttgcggtttcccacgacattcctttcacttctcacttggaagatgagccgtgaggaaatcctgtgttgtgtggtatgtgggctgtgcttctgcttgacttgagggccaagcagcattgcaagccatggttttaaataagaaagaacatttctaaccttcatcttctagtaaggaaacaagtgggctttagagttcttgctcaggaaagacctatgtcccagtccaaccggaccttttactaaagagatcttcctgatcctcctccccaggccaggggaggggtcctccctggggttggagcctttagtagggggtcggagacacgacgtagccttcatgacattcatagtctagttacacgatccctgtaagggtcagttgaagtaagtgctacaaaggaagggaggtgctcagtggagagggctctcttttatgtattatatttctttcatggggagggatatggatcagggatcagcagaggtgtttcagtcccgagggaaagaaagtcagcgtggcttgggagttgggagcagcaagacagtggctcaagatatcttaagactagtggagtacaccttgcatgttaaaagccttgctcagggctgcctggttcttgtaggacgacagagatggcctagctctgcatactgcacccccaggggctcagaacagtgcaaatgtcagtctatctgtcagtggcagagccagccttggagcaggggtgcaaggaggtctctgcactggccaggcatgcagaacattctgttcagtagcactggacagaaggccccatctagatgagacagagctggtggggcaggacaaagactcctggcagctcaaacggcctggcagatgcttggagagagggggcttcttgagacagcaccatttctgggaagagagtcacctgggagggatgaggccacgctccggcttggaggtgaagagaggggctgctgcaagaaagaattagagacatgccagcctttgctgtgttgcccaggctggtcatgaactcttggcctcaagcaatcttcccacctcagcctccccaagcgctgggattatagacatgagcccccatgctggccaataaaagatgattttatggaggggatggtggtgaaggttgtgggtggtatgaaatagtaagaaatatatattggtctgcacccagttcctgccacagagctcctaaaatcctgagaacttcctgggtgagcatcttttgttctaatgaggtgactcttggtggctcctggataggagtgaatcaccagaaagatcaagccagagttagaagcagaaagtgctggctataacacaggaaagctgtaacacaaataataaagtttttttttttttttttgagatggagcctcactctgttgcccaggctggagtgcaatggtgcaatctcagctcactacaagctctgcctcccaggttcaagtgattctcctgcctcagcctcctgagcagttgggactacaggtgtgtgccaccacatctggctaatttttgtatttttagcagagacggggtttcaccatattaaccaggctggcctcaaactccttaccttgtgatccgcctgcctcagcctcccaaagtgctgggattacaggcatgagccaccgtgcctggccaaaagacattgttcttaaaagaatcaactaactaaccaaataaataaaaatctaacctaattaagaaactaaaaatacacaaaaattaatttcaaggggagaaaaatcatgtaaagagagaaagataatgaatactttgcagaaatttatgaacataaacataaaacttggatgaaatgcatttctaggaaaacataatttatcaaaactaaccacaagtaaaatagaagcctaaataggatattttcaagagaagaagtaaagttgtcaaagtgctacccttcaaaaaaacaccaggctcaaacaatctgacatgggaatgttagcacaccttagagagcaaataaaactttgaatgggcttgaaatattccagactctagaaaaacaaaacttcccaattctttttataaagcaagtataaattgataccaaaatcttataaagaccttatacaaaacttcataccaatctcttttatgaatacaaaacccttaataaagtattaccagacagaacccaacaatacataaaaatgtcacatcataacatagtggggtttatttcaataatgcatggatggttcaatacaaggaaattcagtaacacaatataatagatcatgtgaatatacccaaagaaaaaatagattattttcatagatgctgtaaaggcatttgaccaaattcaacacctactttttaggtggtcaataaaataaattagttactccttctttagcatgataaaatatatttatcagcccagaaggcatcattttacccgataagggcacacgctggagggaataatgttaaaattaggaataagaggatagctagtttctttcttcttttttttttttgagacggagtcttgctctgttgccaggctggagtgcagtggtgcaatgttggctcactgcacgccccccgcctcccaggttcaagcgattctcctgcctcagcctcccgagtagctgggactacaggcgcgcaccaccatgcccggctaatttttttttgtattttagtagagatggggtttcaccatgttggtcaggctggtcttgaactcccaacctcacgtactgggattaccggtgtgagccaccacgccagcccaactactttcaacattatccttaatactgatgcttattgacttactatggggttacctctagataaatccataataagttgaaaatataagtaaaaaatgcccttaatacacctaacctaccaaacatcatagctgagcccagcctgccttagctatgctcagacactgacgtcagcctacaattggcaaaatcacacagcagcacagtctactgcagagcatctgctgtttgcccttgtgactgcgtggctgcctgggagcttcccagcttcacaagacagtattacgtagcacatcactagcctggggaaagatcaaagttgaaaatttgaagtgtggtttccattgaatgtgtactgcttttgcaccatcatcaagtcaaaaaattttagttgaaccagcctaagtttgggaccatctttattttcaggaggaacttccatgtacattgatgacggacgatagaatccgtttctatcatcctaatgaacataatgaataaatccagacaaacataaacattaacagagtaagcagctttcggggctggaagccagaagagggtgggagcgcagagagagaggccaaacaccagggctgcttctgctttgcgggtatttgctgatctggacaaggtatctggaaggctgagctaagcctcctttttttttgaggtggcgtctcactctgttgccaggctggagtgcaatggtgcgatctcagctcactgcaacctccacctccctggttcaagcgattctcctgcctcagcctcccgagtagctgggattacaggctcccgccactacacccagctgatttttgtaattttagtagagacggggtttcaccatgttggccaggatggtctcgatctcttgacgtcatgatctgtccacctcggcctcccaaagtgctgggattataggcgtgacccaccgtgccccgtctgagctaagcctcttgagcataggggactaaaaatgaaatctagcgcatgccaagtttagggtcccaggcaattcctttccactttggggtccactttggggtccaccccacccaagaagaaggatgacttggaagtaaaccagctctgaaatatggatggtcctctgggaccataccaatcccttcatatcaaccacatccagttcctcaaaactggaacttggattaagatggcctaggacttctagtgtcccaggagcctggcattgcaaacaaaaatcctctccggaagaagataataccttaagcttcaaatgactctctaataaatttcaaatacaatgtccagcacacaaacacaaattaccaggaacgtgatatgaggcctgatggatgggaattagcagaaacttcaggcatgagaaacataccctcagaggcctagaatctatctagtgtctagataatggagatatgaaatacagacacttaaacaactatgtttcccatgttcaaagaggaaatttgcaaaacttgaaagtgttggcaggaaatcagaaactataaaatgtgacaacagcatactttagagtcagtataaattacggtcccgaaaactgcagaattccagaacttaatggtaaagcaagggtttaacagcagaatagaaatagccagagagaactaggaagtaagtcagatgacactacccagaataaggcactgagaggccaaggaatggaaaatgcagaagaaaggatatggtgagaggatctaatatacatttatttggagtaccagggagagagagaaggagaagaacagaagccgtgtttcaaggacggtgactgagaggcttcgaaactgatgaaagccatcagttcacaaattcaaagcccagtgaattccaaggagaaaaaaagaaatccatactgtgaaagcaagtccagacaatgacaaacaccatcaacaatacacaggacaggcataagatgcatttaatggggacactcagaggcagagggttatcagaaggaggcacttctctcccaagttctcatcatcccagggccagggacagctggtcacaccttagggagttcactaggagagggatctggcttcttgtcattctgggtatttgtagggaaattggaagggaaccgagagcacctagccaatcgcatagcaatgggagatttcaggctgtggggaatgtctttgctggtgaaaagaacatcctgaccttagaaatctttcaccgagggggatctgcgttccagaacttctggagctggtataggtaaggctttgagctttcctactgagccagcctgttgctaggttaccaaaggggacctcgagggccatctggccaacaagcagacttgtctctccttacacccccagacgtatcactgcaaaactacagaaaaccaaagacagagaaaatcttaaaagcagccagatttaaaaaatggcatattagtttcaaagcagcagccatgaaattgacagctgatgtctcaacagcaagaatgaaaagtggaagacaggccaggtgtggtggctcaggcctgtaatcccagcactttgggaggccgaggcgggtggatcacgaggtcaggagaccaagaccatcctggctaacatggtgaaaccccgtctctactaaaaatacaaaaaaattagtcgggcatggtggtgggtgcctgtagtcccagctactcgggaggctgaggcaggagaatggcgtgaacccgggaggcggagcttgcagtgagccgagattgtgccactgcactccagcctgggtgacagagcaagactctgtctcaaaaaaaaaaaaaaaaaaaaaaaaaaaagggtgacgaagcttcaatctcctgaaaggaagcaactgccgcctttgattcgatacccaccaaaatccgtgaagaaggaaggcaaaataaaaacacttcctgattgaactggaaagatttccgcaatagaagacccactgtccaaggaattctaaaggatgctttccaggcagaagaaaatgaccccagaggaagatcagagattcaggaaagaaatggagagtgataaaaatggaaaattcgggggccaatttaaacaaaagctgactgctctacaactgttgtgtctctatcttttgtaacatatatgtgtgtgtagcttttttttttttttttgtcaagatggattctcactctgtcgcccaggctacagtgaaatggcacggtctcggctcactgcaacctctgccccttgggctcaaatgattctcttgcctcagcctcctgagtagctgagattacaggtgcctggcacaatgcctggctaatttttgtatttttactagagatgggatttctccatgttggccaggctggtcttgaacacctgacctcaggtgatccacctgcctgggcctcccaaagtgctaggattacaggcgcgagccactgcatctggcctatgtgtgtgtttatatggaattaaaacacatggcaataataccctccaaattgggagaaaccaaaaatagcatttaaatgttgtaagctccctgcataatcaagaagagaatagatttacgttagattttgatacctggaggatgaatgttgtaatttctagggtgaccatgaaaagaggagacaacggtgtatgtttttttttttttgagatggagtctcactttgtcacccaggctggagtgttgtggtgtgatcttggctcactgcaacctcctcctcttgggttcaggccatcctcccacctaggcctccagagtaggtgggatcacaggcacctgccaccacacctggctaatttttttttttttttaaatatttagtagagatggggtttcaccatgttggccaggctggtcttgaactcctgacctcaggcgatctgcctacctctgcctctcaaagtgctgggattacaggtgtgagccatcgcgcccggccaacagtgatcactttcaaactaacagaggttcaaaaataaaatcagacttaaccaaaaaccaggtaacagagctggtaggatatacagaaagactgacctcacgtatatcaacgattacagttaatattaatgaaggaaatgctctagtttaaaaacgagggttgtcaaagaccccacataagaagctccttaccagcggtgcacctagaacctaaggaaacaggacagatgaaggaggacgcgcccccgccgctgtcctgcgcctcagccatcctatgagacgggaaaggtttctgtctgcagctgggcccgtgctctttaccagctcctggctttcttctctggaaggttcctgcctgttttgccctcacacctgctcctctctcagccctctcaggggtggggctggaggccaccaaagagcctcctctgctctccagttgctcgactgctcctcatttccccctggggtctgcgtcagggtttccttcttttccagccccaccccgcgtgcatcccacctggtctcgggtcggggctgctcccgcttactgccccctgcccaggctggtgtgcaccccctctggctgctttcaaggcctcttctctcttctcggcaggacaggcacaggcaggtggccaggtgtcatgcttagctccccgcccagtgagattctttcatttaacaatcttcccctgaatagttcatgttcattgctgaaaatttgaaaaatatggaaaagcacaaagattaagatataaaccgccctcaattcccctgcccagagagagtcactgctatgacttggtgactaggaaccttatttctctctcgctcttttttttttttttgagacagagtcttgctctgtcacccaggctggagtgcagtggctcgatctcagctcactgcaacctccgcctcctgggttcaagcgattctcctgcctcagcctcttgagtagctgggattacaggcacctgccaccatgcccggctaatttttgtatttttagttgagagagggtttcatcttgttggtcaggcggacttgaactcctgacctcaggtgatcagcccacctcggcctcccaaagtgctgggattacaggtgtgagccactgcgccttcatctctcttctgtgtatgtgtacgctgttttttctttagaatgggggacgttatcaggctctacatggtgtgtagtcggctagcatgttgtaagcctttccctgtgtcacaagtgctcatctggaacaggattctaatgactgcctgtggctatgttgggattcctttaactcagctccttctgcccagcatctatcttttttccatcttttgtcctaagtgttgctataataaatcattgatcacacatgcctgactgtttgcataggataaattacgggaaatgtttttgctgttcagggactgtgcccatttttaggcctcagagacaccatgccagactgcccagtattgatctttactctttttagatgatgccaaacttttctgtgaactttaaaaacctgtgtcttgacagtccatttctgtaagtctttcacattagatttcctgtcaggatgatagtcaattctaggcagatgatgttttctcagccatggctgaagcagttgtgatttgttgtggccatgtaaagtcccgatgatccattgcctccctggatgggttggaataatttggtttgggagcatataacagaatgacctggagtcacagcagctcagacggaagtgtatttctcccttacagatgaaagaattccaggccaggctggaatgacaactgcacacagtcatctgggccccctccttccagctcccatcaccccaggatgtggcttttatgcagatgatccaaaatggctgctcaagtcccagccaacacatcccattccagggagcaggaaaaaggtgtgtctttcccttcattttatgtgattcctttctagaagtactactcattacttctgcttgcatctccctggctagcacttacttagttatatggccatagctagctgaaggaaggacagggactgtcatacactagctaagaggcaaactgcttagataaaaaggtctctaaagaaggtcagagcggctgctagggtgcaactctattacttattgttatgggacgaactgtgtccctcattcaggttgatgtcctaagccccagaacctcagaatgggattgtatttggagacaggttctttaaggaggtaaggaggctaaaatgagatcattagggtgggccataatccgactgatgtcttacaagaagagattaggacacggacatgctcagagggacggccacgtgaggacaccaagaaaggcagctgtctgcaagtcaaggacagggctcaggggaaaccaaccttgccaacaccttcatctcggacttctagcctctaggaccatgagaagatacatttctgttgtttaagctgcccggtctgtggtactttgttatggcagcccaagtaaacaaatacagtcatctgctgctggaacaaatcaccccagcactgtggcttggcagcacacatgtctagtcatagagttatatgtagttacgtgtagagccatatgtatcgtcacacgttctgtgggtcaggaatttggacccagcttaaccagctccacttctcgccagggttcagtcaaataccagctgcctcccacctgagagctcagccggggaagggtccctttccaatctcacgtggtgttggcaggatccagttcctcatggcctgctggactgagaacctcagttctcactgcctgttggccagaggccgcctttatgtcctcgccatgtgggcctctccaacatggcagctgacttcatcagagcatccatgccaagaaggcaacagagagggccagggagactgaagtcatacccttttgcgacctagtcatggggtgacattccatcacctttgcccattggttagaagcaggccaccaggtacagcccaagctcacggggaggggtcatacaagggtgtcaataccaggaggtgaggggtgctggggccatcttatgagtctgcccactgaggtaactaacaaccttgaggcctgacacagtggacaaaggcccttattaacagcagagaactgggaactttatttatttatttatttttgagacagagtctcactcttgtcacccaggctggagtgcaatggcatgatcttggctcactgcaacctccacctcccaggttcaagcaattctgcctcagcctccggaatagctgggactacaggcatgcaccactacacccggctaatttttgtatttttagtagagacagggtttcgccatgttggccaggctggtctcgaactcctgacctctggtgatctgcctgccttggcctcccaaagtgctgggattacaggcgtgagccaccgcacctcgctggaacttaatttttttagagacagtgtcgctctatcacccaagctggagtgcagtggtgcaatcctagctcacttgcagcctcaaattcctgggttcaggtgatcctcccacatcagcctcccaagaactgggaactaacagctgtttctctgctgtccttctcaagaaaagggaggctactgctaccccactggggacaatgctgggtttccctttaggacaggctctgagacaaggcggaggtgctgtttgtggccacagagcaggggactctgggttgcaggtgtggcctggctaaagtaggctttactgggctcctctctgcctgcatcaccccccggctgggcggttgtctctgaggccaaccttactccctgctgggcaggctggacagctgccctctccgtttgcccctctaccacccaaaaggcaggaggctctggagaccaggaccctgcccgccacggcctgtgtcccaggcgtgagggggtgccccacagacctctgctgagctgctgctgaatgacgccccttgggggtcctgccggaaggtcagagcaggggtgcactcccataaagaaacgcccccaggtcgggactcattcctgtgggcggcatcttgtggccatagctgcttctcgctgcactaatcacagtgcctctgtgggcagcaggcgctgaccacccaggcctgccccagaccctctcctcccttccggggcgctgcgctgggaccgatggggggcgccaggcctgtggacaccgccctgcaggggcctctccagctcactgggggtggggtgggggtcacacttggggtcctcaggtcgtgccgaccacgcgcattctctgcgctctgcgcaggagctcgcccaccctctccccgtgcagagagccccgcagctggctccccgcagggctgtccgggtgagtatggctctggccacgggccagtgtggcgggagggcaaaccccaaggccacctcggctcagagtccacggccggctgtcgccccgctccaggcgtcggcgggggatcctttccgcatgggcctgcgcccgcgctcggcgccccctccacggccccgccccgtccatggccccgtccttcatgggcgagcccctccatggccctgcccctccgcgccccacccctccctcgccccacctctcaccttcctgccccgcccccagcctccccacccctcaccggccagtcccctcccctatcccgctccgcccctcagccgccccgcccctcagccggcctgcctaatgtccccgtccccagcatcgccccgccccgcccccgtctcgccccgcccctcaggcggcctccctgctgtgccccgccccggcctcgccacgcccctacctcaccacgccccccgcatcgccacgccccccgcatcgccacgcctcccttaccatgcagtcccgccccgtcccttcctcgtcccgcctcgccgcgacacttcacacacagcttcgcctcaccccattacagtctcaccacgccccgtcccctctccgttgagccccgcgccttcgcccgggtggggcgctgcgctgtcagcggccttgctgtgtgaggcagaacctgcgggggcaggggcgggctggttccctggccagccattggcagagtccgcaggctagggctgtcaatcatgctggccggcgtggccccgcctccgccggcgcggccccgcctccgccggcgcagcgtctgggacgcaaggcgccgtgggggctgccgggacgggtccaagatggacggccgctcaggttctgcttttacctgcggcccagagccccattcattgccccggtgctgagcggcgccgcgagtcggcccgaggcctccggggactgccgtgccgggcgggagaccgccatggcgaccctggaaaagctgatgaaggccttcgagtccctcaagtccttccagcagcagcagcagcagcagcagcagcagcagcagcagcagcagcagcagcagcagcaacagccgccaccgccgccgccgccgccgccgcctcctcagcttcctcagccgccgccgcaggcacagccgctgctgcctcagccgcagccgcccccgccgccgcccccgccgccacccggcccggctgtggctgaggagccgctgcaccgaccgtgagtttgggcccgctgcagctccctgtcccggcgggtcccaggctacggcggggatggcggtaaccctgcagcctgcgggccggcgacacgaacccccggccccgcagagacagagtgacccagcaacccagagcccatgagggacacccgccccctcctggggcgaggccttcccccacttcagccccgctccctcacttgggtcttcccttgtcctctcgcgaggggaggcagagccttgttggggcctgtcctgaattcaccgaggggagtcacggcctcagccctctcgcccttcgcaggatgcgaagagttggggcgagaacttgtttctttttatttgcgagaaaccagggcgggggttcttttaactgcgttgtgaagagaacttggaggagccgagatttgctcagtgccacttccctcttctagtctgagagggaagagggctgggggcgcgggacacttcgagaggaggcggggtttggagctggagagatgtgggggcagtggatgacataatgcttttaggacgcctcggcgggagtggcggggcagggggggggcggggagtgagggcgcgtccaatgggagatttcttttcctagtggcacttaaaacagcctgagatttgaggctcttcctacattgtcaggacatttcatttagttcatgatcacggtggtagtaacacgattttaagcaccacctaagagatctgctcatctaagcctaagttggtctgcaggcgtttgaatgagttgtggttgccaagtaaagtggtgaacttacgtggtgattaatgaaattatcttaaatattaggaagagttgattgaagttttttgcctatgtgtgttgggaataaaaccaacacgttgctgatggggaggttaattgccgagggatgaatgaggtgtacattttaccagtattccagtcaggcttgccagaatacggggggtccgcagactccgtgggcatctcagatgtgccagtgaaagggtttctgtttgcttcattgctgacagcttgttactttttggaagctaggggtttctgttgcttgttcttggggagaatttttgaaacaggaaaagagagaccattaaaacatctagcggaaccccaggactttccctggaagtctgtgtgtcgagtgtacagtaggagttaggaagtactctggtgcagttcaggcctttctcttacctctcagtattctatttccgatctggatgtgtcccagatggcatttggtaagaatatctctgttaagactgattaatttttagtaatatttcttgttctttgtttctgttatgatccttgtctcgtcttcaaagtttaattagaaaatgattcggagagcagtgttagcttatttgttggaataaaatttaggaataaattattctaaaggatggaaaaactttttggatatttggagaaattttaaaacaatttggcttatctcttcagtaagtaatttctcatccagaaatttactgtagtgcttttctaggaggtaggtgtcataaaagttcacacattgcatgtatcttgtgtaaacactaaacagggctcctgatgggaaggaagacctttctgctgggctgcttcagacacttgatcattctaaaaatatgccttctctttcttatgctgatttgacagaacctgcatttgcttatcttcaaaatatgggtatcaagaaatttcctttgctgccttgacaaaggagatagattttgtttcattactttaaggtaatatatgattaccttatttaaaaaatttaatcaggactggcaaggtggcttacacctttaatccgagcactttgggaggcctaggtggacgaatcacctgaggtcaggagtttgagaccagcctggctaacatggtgaaaccctgtctctactaaaaatacaaaaattagctggtcatggtggcacgtgcctgtaatccaagctacctgggaggctgaggcaggaaaatcgcttgaacccgggaggcagagtctgcagtgagttgagatcacgccactgcactccagcctgggtgacagagcgagactctatctcaaaaaaaattttttttaatgtattatttttgcataagtaatacattgacatgatacaaattctgtaattacaaaagggcaataattaaaatatcttccttccacccctttcctctgagtacctaactttgtccccaagaacaagcactatttcagttcctcatgtatcctgccagatataacctgttcatattgtaagatagatttaaaatgctctaaaaacaaaagtagtttagaataatatatatctatatattttttgagatgtagtctcacattgtcacccaggctggagtgcagtgatacaatctcggctcactgcagtctctgcctcccaggttcaaatgcttctcctgcctcagccttctgagtagctgggattacaggcgcccaccaccatgtccagctaatttttgtatttttagtagagatggggtttcaccatgttggccaggctggtcttgaactcctgaccttgtgatctgtccacctcggcctcccaaagtgctgggattacaggtgtgagccaccatgcctggctagaataataacttttaaaggttcttagcatgctctgaaatcaactgcattaggtttatttatagttttatagttattttaaataaaatgcatatttgtcatatttctctgtattttgctgttgagaaaggaggtattcactaattttgagtaacaaacactgctcacaaagtttggattttggcagttctgttcacgtgcttcagccaaaaaatcctcttctcaaagtaagattgatgaaagcaatttagaaagtatctgttctgtttttatggctcttgctctttggtgtggaactgtggtgtcacgccatgcatgggcctcagtttatgagtgtttgtgctctgctcagcatacaggatgcaggagttccttatggggctggctgcaggctcagcaaatctagcatgcttgggagggtcctcacagtaattaggaggcaattaatacttgcttctggcagtttcttattctccttcagattcctatctggtgtttccctgactttattcattcatcagtaaatatttactaaacatgtactatgtgcctggcactgttataggtgcagggctcagcagtgagcagacaaagctctgccctcgtgaagctttcattctaatgaaggacatagacagtaagcaagatagataagtaaaatatacagtacgttaatacgtggaggaacttcaaagcagggaaggggatagggaaatgtcagggttaatcgagtgttaacttatttttatttttaaaaaaattgttaagggctttccagcaaaacccagaaagcctgctagacaaattccaaaagagctgtagcactaagtgttgacatttttattttattttgttttgttttgttttttttgagacagttcttgctctatcagccaggctggagtgcactagtgtgatcttggctcactgcaacctctgcctcttgggttcaagtgattctcatgcctcagcctcctgtttagctgggattatagacatgcactgccatgcctgggtaattttttttttttcccccgagacggagtcttgctctgtcgcccaggctggagtgcagtggcgcgatctcagctcactgcaagctccgcttcccgagttcacgccattctcctgcctcagtctcccaagtagctgggactacaggcgcctgccaccacgtccagctaatttttttgtatttttaatagagacggggtttcaccgtgttagccaggatgatcttgatctcctgacctcgtcatccgccgaccttgtgatccgcccacctcggcctcccaaagtgctgggattacaggcatgagccactgtgcccggccacgcctgggtaatttttgtatttttagtagagatggggttttgccatgatgagcaggctggtctcgaactcccggcctcatgtgatctgcctgccttggcctcccaaagtgctaggattacaggcatgagccaccatacctggccagtgttgatattttaaatacggtgttcagggaaggtccactgagaagacagctttttttttttttttttttggggttggggggcaaggtcttgctctttaacccaggctggaatgcagtatcactatcgtagctcacttcagccttgaactcctgggctcaagtgatcctcccacctcaacctcacaatgtgttgggactataggtgtgagccatcacacctggccagatgatggcttttgagtaaagacctcaagcgagttaagagtctagtgtaagggtgtatgaagtagtggtattccagatggggggaacaggtccaaaatcttcctgtttcaggaatagcaaggatgtcattttagttgggtgaattgagtgagggggacatttgtagtaagaagtaaggtccaagaggtcaagggagtgccatatcagaccaatactacttgccttgtagatggaataaagatattggcatttatgtgagtgagatgggatgtcactggaggattagagcagaggagtagcatgatctgaatttcaatcttaagtgaactctggctgacaacagagtgaaggggaacaccggcaaaagcagaaaccagttaggaagccactgcagtgctcagataagcatggtgggttctgtcagggtaccggctgtcggctgtgggcagtgtgaggaatgactgactggattttgaatgcggaaccaactgcacttgttgaactctgctaagtataacaatttagcagtagcttgcgttatcaggtttgtattcagctgcaagtaacagaaaatcctgctgcaatagcttaaactggtaacaagcaagagcttatcagaagacaaaaataagtctggggaaattcaacaataagttaaggaacccaggctctttcttttttttttttttgaaacggagtttcgctcttgtcacccgggctggagtgcaatgatgtgatctcagctcactaaaacctctacctcctgggttcaagtgattcttctgcctcagcctcccaagtaactgggattacaggcgtataccaccatgcccagctaatttttgtgtttttagtagagatggggtttcaccatgttggccaggctggtctcgaacttctgacctcaggtgatccactcgcctcagcctgccaaagtgctgggattacaggtttgggccactgcacccggtcagaacccaggctctttcttatacttaccttgcaaacccttgttctcattttttccctttgtatttttattgttgaattgtaatagttctttatatattctggatactggattcttatcagatagatgatttgtaaaaactctcccttcctttggattgtctttttactttcttgatagtgtcttttgaagtgtaaaagtttttaattttgatgaagtcgagtttatctattttgtctttggttgctgtgcttcaagtgtcatatctaagaaatcattgtctaatccaaagtcaaaaaggtttactcctatgttttcttctaagaattttagagttttacatttaagtctgatccattttgagttaatttttatatatggttcaggtagaagtccaactttattcttttccatgtggttattcagttgtcccagcactgtttgttgaagagactattctttccccatggaattatcttagtacccttgttgaaaattaatcgtccttaattgtataaatttatttctagactgtcagttctacctgttggtctttatgtcgatcctgtgccagtaccatacagtcttgattactgaagtttgtgtcacagtttaaattcatgaaatgtgagttctccaactttgttccttttcaagattgatttggccatgctgggtcccttgcatttccgtacgaattgtaggatcagcttgtcagtttcaacaaagaagccaagtaggattctgagagggattgtgttgaatctgtagatcaacttggggagtattcgcatcttaacaatattgtcttccacctatgaacatgggcaaactttgtgtaaatggtcagattgtaagtatttcgggctgtgtgggcacagtgtctctgtcacagctacgcggctctgccattgtagcatgaaagtagccataagcaatatgtatgagtgtctgtgttccaatagaattttattaatgacaaggaagtttgaatttcatataattttcacctgtcatgagatagtatttgattattttggtcaaccatttaaaaatgtaaaaacatttcttagcttgtgaactagccaaaaatatgcaggttatagttttcccactcctaggttaaaatatgataggaccacatttggaaagcatttctttttttttttttttttttttttttgagacggagtttcactcttgttgcccaggctggagtgcagtggcgcgatctcggctcactgcaacctctgcctcccaggttcaagacattctcctgcacggcctccctagtagctgggattacaggcatgcgccaccacacccagctaattttgtatttttagtagagacggggtttctccatgttggtcaggctggtcttgaactcctgacctcaggtgatccacccgcctcagcctcccaaagtgctgggattacagggtgtgagccaccacaccctgctggaaagcatttcttttttggctgtttttgttttttttttaaactagttttgaaaattataaaagttacacatatacattataaaaatatcttcaagcagcacagatgaaaaacaaagcccttcttgcaagtctgtcatctttgtctaacttcctaagaacaaaagtgtttcttgtgtcttcttcccagattttaatatgcatatacaagcatttaaatgtgtcattttttgtttgcttgactgagatcacattacatatgtatttttttacttaacaatgtgtcatagatattgttccatagcagtacctgtaattcttattaattgctatgtaatattttagaatttctttttaaaagaggacttttggagatgtaaaggcaaaggtctcacatttttgtggctgtagaatgtgctggtgacatattctctctaccttgagaagtccccatccccatcacctccatttcctgtaaataagtcaaccacttgataaactacctttgaatggatccacactcaaaacatttagtcttattcagacaacaaggaggaaaaataaaataccttataaagcactgtttaatattgtattaaattggatcaatttgggggctagaatgtatgttagagacatgatatgtccataggtccttgctatcacagtgaggtctcagggacagtcgtttggtatcatttgggatctcataagcagactctctctgcttgacctgacaaatcagagtctgtgttttaacaggttcagtgagtgacttacatgcacattggagtttgggaagctccactgtaggtgcttagaccttacctttgttgttgctaataacaatgcaagcatttgggaggaagacctgtgttgctcatatgtgtccaggtgtagctgaggtggccttgcttatctgctgtagggccgttgagcatttctgtagctgtgatgagtgagctgaggtgagcctgcggagagctcccagccattggtagtgggactcgcttagatgaactggaaggaccctttcatctgagcagccactatggagaaaaacaaccgaatgaggggagagacaatgtgcaattttatttagggcacaaaggagagctgtggttagaaggtgacatttgagtggaaagggggcaagccatgtgtatagcgggagaagagaggtccaggcagagttaacagaaggcagaaatgctttccatgtttgagaaccagtaaggaggccagtggctgaagtaaggtgaagggcagaaataaggatgaggctgcgagagatgagaggttagagacgagcgtcttgtgcaccaagataagcttgtgtggtcaaaacaagtagtttaatttatgtttttaaaagatcattttggctgggcacaatggttcatgcctgtaataccagtagtttgagacggtgtggtgggaggattgcctgaggccagacgaccagcatagccaacatagcagcacctataaggtctctacaaaaaactttaaaaaattagctgggcatagtggtgtgtgcctgtagtcccagctactcaggaggctgaggaggctggaggattgcttgagtccaggagtttgaggctgcagtgagctatgattatgccactacactacaacctgggcaagagagtgagaccctgtctctaaatatacacacacacacacacacacacacacacacacacacacacacacacacacacacacatatatatgtatatatatgcatttagatgaaaagatcactttgacaataccacatgctggtgaggatttagaaaaactaggtcacttattgctggtgggaatataatatagtacggccactctggaaaacagtttggcagtttgtcataaaactgaacataccgttagtatacagcccagcagcaactacaatcctgggcattaatcctagagaaatgaaaccttaatgttcacataaaaacctatactcaagtatgcatagcagctttacccataatatctaagaactggaatcagctcagatgtccttcaacaggtgaatggttaaactactcagtaataaaaaggaatgagctactgatagcatgcaacagtttaggtgaagttatgctaatgaaaaaagccaatcccaaaaggttatacatactgtatgattctatgtttttttgcaatggcacagttttagggatggagaatagattagtggttgcctggggttagagatggggtagtagagtaggttagtggtggcagaggagagaaaagagagggaggtgaatgtggttataaaaggacaacacaggggaatacttgtaatggaaatgctttgtctttttttttttttttttttttttggcgacagagtcttgctctgttgcccaggctggagtgcagtggcatgatcttttctcactgcaacctctgcctcctgggttcaagtgatacttgtgtctcagtctcccatgttcagagtgaaacaaaccagaggtaatgttcatccaaataatccaacacacatgacattaaaacatcaagatcaggtcggacgtggtggctcatgcctgtaatcccagcacttttgggaggccaaggtgggcagatcacttgaggtcaggagttcgagaccagccgggccaacatgatgaaaccccatcttgactaaaaatacaaaaattagccgggcatggtggtgtgcacctgtagtcccagctacttgggaggctgaggcaagagaactgcttgaacccgaggggcagaggttgcagtgagctgagagtgcgccattgcacttcagcctgtgtgacagagtaagactccatctccaaaaaaaaaaaaccaagatcaattaaaatacagcattactgggccgggtgtggtggctcacacctgtaatcccagcactttgggaggccgagatgggcagatcacgaggtcaggagatccagaccatcccggctaacacggtgaaaccccgtctctactaaaaaatacaaaaaattagccgggtatagtggtgggtgcctgtagtcccagctacttgggaggctgaagcaggagaatggtgtgaacccgggaggcagagctggcagtgagctgagatcgcgccactgcactccagcctgggcgacagagcaagactccgtctcgggggaaaaaaaaaaataaataaatagaatgctgtagtgtccttgagtttacatgcccctccttacgcttgtgtgcccgtgcagattgcttgattacacaattagaggaggctggcggaggattgttttaatttttttttttttgagacagtctggctctgttccccaggctagagtgcaatggcgcaatcttggtgcactgcaacctctgcctcctgggttcaagcagttcttctgccgcagcctcccgagtagctgggattataggcgcccgccaccacgcccaactattttttgtatttttagtagagcagcgtttcaccatgctggccaggctggtctcgaactcctgacctcagatgatctgctgccccagcctcccaaagtgctgggattacaggcgtgagccacacctggccgtttgttttaattttgaaggtgaagtgaaagtgactacatttaccaaaagtgattgaaaagccaggactgttcttaccctgtttttccagttcttgctcagagcaaggtggtttctttttcacttaatcaccatacttacttttcatgtagaacaagtcagtttgagttatcagttcatcatcttaactaaattccatgggggaaggaattagttttagtttcttaaacttccaggtttgcttattggacaaaatgagatagcaaggcagtgtttttaagttagattttttatttctttggtaatacaattttctcagaaacttagtagtcttttagtttagttgtttttagttggtcctatgttttggatcacccctctctactttattttgatagtgccaactgtgaagacatctgaagccataggtttggatgggaaggaggcatctttagcctgatcatcttcgccaggctgtttatctccttttgcttggctgagaagtcttaataggaggcttattcccagctatttggggacatagaagcagttagccattgcttatattttactgaggtctgtgtggtatgttgattgtagtcagttaacgattttgagaactgaaggcagcctggtatatatagagtaggtattagactgtgtttcttctaattgaatttcccatctcttgtaatctatgccatcatcttctgtactgctgagaaagaaagaaagtttctaatcaaactataccactggttgtaagatgcagtttggctttagtgatgttaacacatgattcaaacgtgaaattgattgagtattggtgaaatacagaggagatttaaagccagaagacctgggtttaaatgctggctgtatgacttcatatctgtgtgatcttgggcatgtcatggttggcacttcaatttcttctctctataatgggggaagtgaggccagtcatggtggctcatacctataatcccagtgctttgggaggccaagatgggaagatcgcttgaggccaggagtttgagcaattgggcaacatcgtgaggccccgtctctacaaaatattttgaaaaaattagccaggcccagtggtgcgtgcctgtggtccgcgccactcaggaggctgagacgggaggatcctttcagcctaggagtttaaggctaaagtgagccatgattgtgctatcgtactccagcctgggcagcagagcaagatcctgactctaaaaaaaagtaaaataaagtaaaatgggggaaatgaactgctttagtaacatcatctgttttttctgtgagcagcgtagcttgacagccattggtgaactcgtgccctgtgcttccctgtccagatccccattctgcccgcaacatggagtataacggtttattcatagtagtcgagaaacactcactgaatgaatgaatgaggtgtagaactaagtggagtgggtaattcaacacatattaatttccttcttttttttatttttagaaagaaagaactttcagctaccaagaaagaccgtgtgaatcattgtctgacaatatgtgaaaacatagtggcacagtctgtcaggtaattgcactttgaactgtctagagaaaataagaactttgtatattttcagtcttaatgggctagaatattctttgtgtcccagctattttaaatggattcagaaatccatttaagatgaagaaggacccttttcccatatttctggctatatacaaggatatccagacactgaaatgaataatgttccctttttgtaatcttttatgcaaaaattaaaaccattatggtaattgaacaacatgtttatgtttagttaacacccttagcaactatagttattttaaaaccatctatggtttgatatttttgcatttgttgcaatagtaggaacagcacaagacagttcagtttgtctctcttatttgctttttcttggcagtttgctgtcctattgtacctctgctcctagcagtggctggagcccactcctctgtgcttcgggattagtggggatcgtggggcattgactgtaggtcagctttccttgcttgatctttctcactgggatgaactagcagcaccttcttttgtagctgctttgcttttgactatctttctgaccgttgttcctagtagctgtagatggtaaatatatttaggcctgtttccaatggctcagtaggagacatattcacctatgatatctgaattctgttacccacatgggcatgcgtgaaatagttgccttgccttactttcccttggaataaataattcatgttattctcctggtagaagctagaaaaagcctttatagtcagtcagaaaaaaatttttagacaaataatcttgattttagtactgacaaaaacgtgtggtgattctttttttaatttttttttgagacggagtttcactcttgttgcccaggctggagtgcaatggcgtgatctcggctcactgcaacctctgcctcctgggttcaagtgattctcctgcctcagcctcccaagtagctggagttacaggcatgtgctactgtgcccagctaattttgtatttttagtagagatgttggtcaggctgatctcgaactcccaaccttaggtgatctgcccgcctcagcctcccaaagtgctgggattacaggcgtgagccagggcgcccggtgattcatttgttttttcaaaaaatttcctcttggccattgcttttcacttttgttttttttttttttttgagacggagtcacgatctgtcacccaggctggagtgcagtggcatgatcttggcttactgcaagctctgcctcccaggttcacgccattctcctgcttcagcctggcgagtagctgggactacaggtgctcgccaccacacccggctaattttttgtatttttagtagagatggggtttcaccgtggtcttgatctcctgacctcatgacccgctcaactcagcctcccaaagtgctgggattacaggcgtgagccaccgcgcccggccctctcttgtctttttattgtggtaaaatgcacataaaattgactgtcttaaccatttttaggggtacagttcagtatatatattcgtaatgttgtacagccatcactgccatctacttcataagtttttcttctgtcaaaactgaacatctgtcttcattaaactccctatcatccattctttcctgtagtccctttctactttctgtctgtatgagtgtaactgctctggagacctcatgtaagtggattcctacaggatttgtgttttttttttggtgatctgcttatttttaatgcctctgtgcatttgtattatatactttcaaagtgatttcacaaaaccgtttcattttaggttaactcatttctgttgtttgtgaaatactgtgtatgattctgttctgtttctgtctaatttgtggaaatgttgtgggaagaaaatgaaataacaaatgagcatatgtcctgaaaataaaaatataaaaattctaagttagcatgctattgtagaatacaacgctatgataaaagtaggaaaaaaaaaggtttgaattctatctctgctacctgtgtaagctgggtgactttagataagctgtaacgtgtttgagccttactggctcatttttgaaatgtaatccctagttacacagttcttgtgggatcagatggtacatgtgaaacactgtgaaaaagcaactgcatagatatgttcattagccacctgagcgggaagcgtatcccattgcgatgcccatcatccaaagctatatgttatctttacttttttttttttgagacagagtcttgctctgttgcccaggctagagtgcagtggtgcaatctcagctcactgcaagctccacctcccgggttcacgctattctcctgccccagcctcccaagtagctgggactacaggcacccgccaccatgcctggctaaatttttgtatttttagtagagatggggtttcaccgtgttagccaggatggtcttgatctcctgacctcgtgatccgcccgcctcggcctcccaaagtgctgggattacaggcgtgagccactgcccctggccatctttactttttttgtgaaatgactttaaatacttggcaaacatttggtcattgttcatctgatctccaccatccaggtctcagagaacataatttctctctgaaagcttattgacccaggaaataagatctctttcaatctgagtgcgtcaggctttattcttgtcattttgtcttttgataattttcaaatggaattcatggaatgttggcttatattcatatattagtaaagtatgttgagacatcttaagattgatttgtggttctatatgccatattaaatcaaaataatagctgttaatggttttcacattagtctgtctcttgtttttatggagtaatgctgagagttcattatgcttgttctacagaagagcatgttaaaaggagtttttggagtcagagaggttattcttggtttcataggatacactctatactttttagggatttcagagtatatagctgaaggtgatattttatgtaaatatgttttatggaaacttattgctcatcgctgtttcctgttaactctcctaaaatataattaaacttttggaacttttttatagcttttgtgctagactaatttttgtctctaatgaggttatataaatggcagcttctgacgttttcaatgtaggaagtcatttaaaacttcatgtatattgtgaaaatgtagtctgctttaagctctctaaagtggtctaagttactggttcctaagtatggatgagcatcaaaatcatctggaaaatttgttaaaaatacagtaatgaaggcacctcactgtcctttttcccaaacatacttctgcattctgtttgagtaggtagggactacacatttttcacaagtatcctcttgggaatacccaggaatgcttacttgagcaacctcttactaatatgtaccttgataaggtggctaggtaaacataaatatacaaaaatccatagatctcccatatattagcataaatcagctagaaaatataacgtttaaagatctagttcacagtagcaccaatatatcgaactctaaggaatcgataaatatgcaaaaactttataaaaacttctgttaatgtttctgaaagatataggtgaccactttctagataggaagattttatattactaagttgaattttctctaaattaacacagaaatttaaaataatcttgatcaaaattctagtagaggtatttttgaacttgttcactgcaagaataaatacataattgcaaagaatatctcaaaatcatcaccaggcctggtgtggtggcccatgcctgtaatcccagcactttgggaggctgaggcaggcagatcacctgaggtcaagagtttgagaccagctggaccagtgcggtgaaacactgcctctactaaaaatacaaaaattagctgggtgtggtggtgcatgcctgtagtcccagctacttgggaggctgaggcaggagaattgcttgaacccaggaggtacaggttgcggtgagcctagatcgcaccactgcattccagcctgggcgacaagagcaaaattctgtctcaagaaaaaagagaaaaaagaaaaagaaatcaacactaatatggtgagacttaatgtatgtgacattaaaatagtgattggatgttaaaacaggtatagaacagaaagaagagtgtatgtgtgtatctgtatgaatttatgatgggtgtaacatatatgtattagggaaatgagggaaatgatacatttctctgactttgggagaacattatatctctacctcatattgcaaacaaacataaagttcagattaattacctaaatgtgaaaaaatgaaataatttctttaaaaaatgtaatcttagtttgaggaaggttaacattataaaggaaaaaactgttttgagtggaatatagttcaatatgtcaaaatccaccttcaacaaaattgaaagtaaattgaacttggggaaagtattgacagcatatagatcaaaggttactagcctgtgtaaagagcagttataaatatcgttaagaaaaacactgtcgacctgtcggcaccttgttctccgactcccagcctccagaactgtgacgagtaagtgcttattgtttaaaccacccagtctgtatgtggtattttgttatagaaactcaagctgattaggacactagtaatcagtagactgaaactgaaacaaaaataagaaccttttttacctgtcaaattggcaaacattaagaatattcagatttttgtcagaggtgatacaaccttctaagaaggcaatttgggaaaatataaagctttagattattatatgtctgacctagcagttttacctctagggtgcttacccctaggaaagtgtgtaatgatattggtgcagtgcccttcatcccattagaaaattaaaaataaccttaatggcctaccactaaaaggggattgaaaatttaagatatatttatttatgtgtttattgagatggagtcttgcactgtccgcctgggccagagtgcaatggtgcgatctcggctcactgcaacctctgcttcccgggttcatgtgattctcctgcctcagcctcctgagtagctgggattacaggctcacaccaccgcacccggctaattttttgtatttttagtagagatggggtttcactgtgttggccagactggtctcgaactcctgacctcatgatccgcgcccctcggcctcccagtgttgggattacaggtgtgagccactgcgcctggccagatacatttatacaagagaatgttagttaacattcatagatatttatattttgtttactttttattaaaaaaattttttttagagacaggatcttactctgtcacccaggcaggatgcagttgcacaatcatagcccactgcagcctgaactcctgggcttaagtgatccttctgcctcagccttttgagtacctgggggactttaggcagtgctactatacctggctaatttttaaatgttttatagatgagatcttgctgtattgcccaggctggtctagaattcctgggcccaagtgatcctcccaccttggcctcccaaagcgctgagattacaggcatgagccaccacttctgaccaatagatatttatatttgtgactggaaaatatattaacaatgtgttaaaaaattcagttaaaaaataatgaaagatttttgcttctggctaagatagaataacaaggacagcatttatcttcttgccttgaaatagttgaaaacggaagaaatatatgtaacagtggttttcaagttattgggcatcaggcaaagaagaatagttatcccaggaaaatgaatgtggagagccctacaatttccttacattactgcctggtcatggcaagaggaaaaactgagaggagactgaggctgagccagtggtttgctgggttgaggaggcagagctgggagtgcagagatgcaaggtggtgagagcccatatggaagaataccagggaagagagctgcagagggagctccggagacctgcaccctgccctctcagtaccctgtcatgtgtgtagctgagtactgacgagcacttgcttgtgcggaaatgacccagggctggaggtagagccacctgaaaggattagaaggaacagttgctgaaagtcacacagggccaggaagaatttctaatcacaccagttggagtggaaaacctcagctctcatagagcaggtagggtactcagaagggtttgcccacctagccccagactaagtttcgttactctgaccctacctaatattaaaaagagattaattaaattgttcgcaacaaaaataatatatttcagtgtttgtaacacgtagaagtgaattgtatgacaatagcataaaggctggaagagcagaaattgacatgtatttgcgctgggcagaataatgctcccctctttccccaaaagatatcaagtcctaatccctggagcctgtaaatattactttatatggaaaattgttttatgatgtgattaaattcaggatcttgagatgagggggctatcttggatgatctgggtaggcactaaatgcaatcacatatatataaaaaggaggcagagggagattttacacacagagagaaggccctgtgaagatggaacagaaagatttgaaggtgctggccttgaaaattggagtgatgaagctataagccaaggaatgcagcagccaccaaagctggaagaggcacggagcagttctcatttagagcctactccagagggaatgtggtgctgccaattccttttttttttttttttttaagatatcatttacccctttaagttggttttttttttttttttttttttttagtatttattgatcattcttgggtgtttcttggagagggggatttggcagggtcataggacaatagtggagggaaggtcagcagataaacatgtaaacaaaggtctctggttttcctaggcagagggccctgccacgttctgcagtgtttgtgtccctgggtacttgagattagggagtggtgatgactcttaacgagtatgctgccttcaagcatctgtttaacaaagcacatcttgcaccgcccttaatccatttaacccttagtggacacagcacatgtttcagagagcacggggttgggggtaaggttatagattaacagcatcccaaggcagaagaatttttcttagtacagaacaaaatggagtgtcctatgtctacttctttctacgcagacacagtaacaatctgatctctctttcttttcccacatttcctccttttctattcgacaaaactgccaccgtcatcatggactgttctcaatgagctattgggtacacctcccagatggggtggcggccgggcagaggggctcctcacttcccagatggggcggccgggcagaggcgccccccaacctcccagacggggcggcggctgggcgggggctgccccccacctcccggacggggcgggtggccgggcgggggctgcccaccacctcccggacggggcggctggccgggcgggggctgccccccacctcccggacggggcgggtggccgggcgggggctgccccccacctcccggacggggcggctggccgggcgggggctgccccccacctcccggacggagcggctgccgggcggaggggctcctcacttcccggacggggcggctgctgggcggaggggctcctcacttctcagacggggcggctggtcagagacgctcctcacctcccagacggggtggcagtggggcagagacattcttaagttcccagacggagtcacggccgggcagaggtgctcttcacatctcagacggggcggcggggcagaggtgctccccacttcccagacgatgggcggccgggcagagatgctcctcacttcctagatgggatgacagccgggaagaggcgctcctcacttcccagactgggcagccaggcagaggggctcctcacatcccagacgatgggcggccaggcagaaacgctcctcacttcctagacggggtggcggctgggcagaggccgcaatcttggcactttgggaggccaaggcaggcggctgggaggtgaaggttgtagtgacccgagatcacgccactgcactccagcctgggcaacactgagcactgagtgagcgagactccgtctgcaatcccggcacctcgggaggccgaggctggcagatcacttgcagtcaggagctggagaccagcccggccaacacggcgaaaccccgtctccaccaaaaaacacgaaaaccagtcagacatggcggtgcgtgcctgcaatcccaggcacttggcaggctgaggcaggagaatcaggtagggaggttgcagtgagtagagatggtggcagtacagtccagccttggctcggcatcagagggagactgtgcgagggcgagggcgagggcgagggaattccttaatttcagtttagtgatactaattttggactctggcctctaaaactgtgaaagaaaaaattttttgtttgtttgtttcttttaagccacatagtttgtggtaatttgttacagcagctgcaggaaactaatttatgctgcatgtgaaatggtgtaataaggtagattgtgatgaagatacatagtataaacaattaagcaacaactaaaagcacaacaaggaattatagctaatgaaccaaaaaaggagattagaataataaaaatggtgaatcccaaagaagccagaaataggggaagaggcaaataaaggaaagaaagagcttgatggtagatttcaacctaactatgtcaaaaaggacattacatgtaaaaggcagcgatttttcagattgaatggaaaagtaagactcggtatatgctgctgcctgcaagaaacacattctaaatataaaggcaaaaataacctacaggtaacagaacggaaagaagttcactgtgcttacaagaattagatgcaagctagactggttctgttaatatcagacaaagtggatttcaaagcaaaggctcttgcccaggatgagatggtcatttcataatgatgaaggggattcgttcatcagcctggcatagcaagctgaaatgtttatgcaccggactacagagctaaaatacatgaagcaaagcctgacagaactacaagtagaaacagacaaatccacagtgatagagatttcagtagccgctctcaatgatttgtagaacacgtagccataatatctggatctagaacacttgaccaacactgtcccctgtgcaacctcattggcatttacaggacactccacccagcaccagcagaagagacactctctcaagtgctcacagaatgtttgccaagatagagcagatgctgggccataaaacaagtctctaaattaaaagcattcaaattattcagagtatgttttctgacctcagtatcattaagttggaatatattataggaagataacctggaaaagcctcagatatgtggaaaaacccatttccacatggcccatgggtcagaagtgaagtcaaaagggaaatttgaaagtcttttggattgactgatataaaaacaatagatttctaaacttgtggggtgctgttacagcatagtaaatggaaatttctagcattaaatgcctgttttaggaaagaaagatttcaaatcaatgacctcagcttctacctttggaaacttgaaaatgacaagcaaatggaatccagagttaccagaagggccaggtacggtggcttatgcctgcagttctgccactttgggaggccgaggcaggtggattgtttgagactggcagttgaagaccagcctgggcagcctagggagaccccatatctacaaaaaacaaaaaaattagccaggtgtggtggcatgtgcctgtagtcccagctaaccaggagtctaaggtgggaggattgcttgagtctgggaggttgaggctgcagtgaactgtgattgtgccactgtgttccatcctgggcaacagaatgagaccctgtctcaaaaacaaaaacagttactagaagaatggacatcataaagataggagcagaagtcagtaaaatagaaaacaaaaatacataggaaatcaataaaaccaaaagctggttcatcaagaacatcaataaattggtaaagctgataggaaaaacagtgaagtcacaaattagcaatatcaggaatgagggagatgacagtagtatagattatatagatattaaaaggactgtatgaggcaggtgtggtggttcacgcctgtaatcccagcaccttgggaggccgaggtggacagatcacctgaggtcaggagtttgggaccagcctggccaacatggtgaaactctgtctctactaaaaatacaaaaattagttggtcgtggtgctgtgtgcctgtaatcccagctacttgggaggctgaggcaggagaattgcttgaacctgggaggcggaggttgcagtgagctgagattgtgccgttgcactccagcctgggtgacagagcaagactccatctcaaaacaaataaataaataaaaaggactatatggtaatattatgaacaactttatgccaataaatttgacaacttatagatgaaatggatgagttccttgaaagacacagaaactattaaagctctctcaagaagatatagataagctgattagccctatatctattttattgaatttaaatgtaaaaatcaatatttagttactggaaaacttttaagtgtggttggaaatggtatacgaactttttcaactgaattttatgaagtctaatcacaggtaaaggttttctgatgaaaatttagtgtctgaattgagatatactgtaaaaaatgttatatatcttaattatttcttcacattaattacatgttgaaataatactttgggtgtattgggttaaattaaatattatgaaaatcttgcctgttttctttttacttttgatgcgtcagctaggaaatataaaagtgtagctcacattctgtttctgttgacagtactgctttggagcacagtgtttgaatgatctatcatttcaaagacctttcctcagttcgttattcatggctgtctgtattccacatagataaggtctgaaatactgctaagtggcatgttttgttttatgcttttataagtttgttgatcattactgatgtggacttttggtgcctcttaggctcattgctatcttccaaccattgtttgcaatttttacctagagataaagagaaagagacatttggtttcagagtagttagattgggatcatgaaagagcaacctcattttgatgcttcaaaaatagcacatcccccgtattactgggatttgctattcttgggattacttcaagaacatccttgtgttactggtttggatgcttctgaatgctgtgaagtcagtttcatgtacatggctcatcagtttagctctctcttggctttgtttagacagttggagcatgatggcctaaacagcttctttcaattaaacattttaaaatagtttacaaatagtaaacaaactccagtttttgtgactctttgtctcgcacaacaaaaacacaatctgaccatgatcatctggcatcttagggtgaaatatggttatactttggcccataccgaaagcaagattaaaaaggggcaggagagatagactgctgaactgattttcaaggttccaagaatattgtaggttaagagtaaaagtaaacttttggtagaaagcagtgggttgtctaggattgaagtatctgaagtttttaaacgaaaatttaaaaagaaaaatgagaattgccttacaagtacaatctcttcttttttaaaaaataaactttattttgaaatagttttagatttatagaaaaaaattagatagggtaggaagttttcatataccctacatccagttaccccagttattatcatcctaatttagtgtgagacattttcatgtttaatgaatcaatattgatatgctattaacttaagtccagactttattcagattttcttaatttctatgtaatgtcctttttctgttccagaattccatgcaggacaccggatacctcattacatttcattgtcatgtcaccttaggctcctcttgacagtttctcttctttttttgcttagaaattctccagaatttcagaaacttctgggcatcgctatggaactttttctgctgtgcagtgatgacgcagagtcagatgtcaggatggtggctgacgaatgcctcaacaaagttatcaaagtaagaaccgtgtggatgatgttctcctcagagctatcattgttgtaggctgagagaagaagcgatcattgagtgttcttctgttttgagtccctgaggatgtctgcacttttttcctttctgatgtatggtttggaggtgctctgttgtatggtttggaggtgctctgttgtatggtttggaggtgctctattgtatggtttggaggtgctctgttgtatggtttggaggtgctcttgtatggtttggaggtgctcttgtatggtttggaggtgctctgttgtatggtttggaggtggtcttgtatggtttgcaggtgctctattgcatggtttgcaggtgctctattgtatggtttggaagtgctcttgtatggtttggaggtgctcttgtatggtttggagatgctctattgtatggtttgcaggtgctctattgtatggtttggaagtgctcttgtatggtttggaggtgctcttgtatggtttggaggtgctctgttgtatggtttggaggtgctctgttgtatggtttggaggtgctcttgtatggtttggaggtgctctattgtatggtttggagatgctctggtatctgcctgcattgcttgccacacctgcccggtcagaaggcgctatgttgacaattgtgcctgcacggtgcctaggtcaatgaagggaaccgatggtagccactggatgctcctgggaaaatgtcactacaggcaccagagaagccagagctatgcccaaatttctatgagtctcagttttcttaaccataaaatgggatcaatgtttttgtggcatgtgtatgagtgtgtgtctgtgtatgtgtgaggattaaattgtgtatgtgtgaggactaattgccactactggatcctcaaagtggtaagaagtgttcttattaataatgacatccttacactcttacccagcaagattgatgggtgtggcactgcttctctttttccatcacatggtttccatggtatccttttgcccagggaatctttgctttgtggctagcactttgttgtttggctaatcacgctttctgtggtcaggacgctggcttctctggagccatgggattctagctccctgtcttgtccctagagtggtcactgtcttctctctccgcttgcaattcctgctttgctcgcatctcacttatgcagtgacgtatatcagtttcaccttgttctccgtgcctgctgatcattggcaccacttgcatggtgccatttagggcctgcttccagttaagcttgcttctccacaggcctaaatatccttgcttgcttcttttattctcactggcaggaccagggcggtctgtctttgcatgagacagggtctcgctcagtcacccaggctggagtgcagtggctgatcacggctcattgcagccttgagctaccgggctcaagctatcctcctggcttggccccttgagtagctgggactacaggcgtgcaccaccatgcccagctaatttttaaaattatttgtagagatgggatctcgccaggttgcccaggctggtcttgaacgcctgggctcaagtgatcctccctccttggtttcccaaagtgctgggatcacaggtgtgagccactgtgcctggcccttgatgtttcagttcttgatatttgatcctcagagtcagaaaatctaaaaagagggctatcccaggttgccttggttcatggcaaatgggacgttaagagggcagagagaatatgaacagaaactgttctaatattggtcatttaatgtgtaagtattgttcttttttaaacctccttcattttttttccaggaattgctggacacagtggcttggtgtgtgtctgaggactgtaggccatggccctaggttgtggttttaggtctcaggtgctcttcctggctgtctccttgcttctttcccatgtcctcttctttgtttccagccatttctcccttatgcttaagtttggtgcagcagggtttggctgctctcagattcctgcttcctcagatgctgtagttgtcaggcccagcgggctggcagcgggatcaggatctggctaggtttgctctcactgtggcagagtagggggaggcgtgggagagcacgtgtgaccccaggccagctgtagggagcataggcatggtcacgtagccttcaggtcctagactttgtcttctcatgagtatggctgtgtgtgtatggtgaaaactaggttctacttagcccaagaaaatgggcacattttgcatgtggtttctgtagagaaatgcactgggtatctgacatagcctggcagcatgcctccctcaggtaggttagtctcaggcggtgaagcacgtgtgtccagcaagaacttcatatgtggcataaagtctccgttctgtgaggtgctggcaaatcaccaccaccgtcaagaggctgaagtgatttttgtctagggaggcaggaaaggcttcctggagtcagcagccagtaggtgaaagagtagattggagaccttcttaatcatcaccgcctcttgtctcaaggggtgccaggaagctgtggaggctgaacccatcttatgctgccagagagtgggacaccatgagggtcaggtcaaggggttgtaccttgtttggtagagaattaggggctcttgaagactttggatgtggtcaggggagtgtatcatttaggaagagtgacccggtgaggacgtggggtagaggaggacaggtgggagggagtccaggtgggagtgagtagacccagcaggagtgcagggcctcgagccaggatggtggcagggctgtgaggagaggcagccacctgtgtgtctgcggaagcaggggcaagagggaagaggccagcagcgtgctgccatcacccagcgactggcgtagattgtgagagaccattccctgctcttaggaggggctgagttttagttttctcttgttatacaataagcttggtatttgtttacaaaacatttgtaaagctaaatcaaggtttgataaggcttctagttttatttaagaagtaatgttgaaataaatgtttgtccaattcgctttgctcatttaaggactttcagtacaaactgcaacaacaggattaggatttaaacgtttctgagatgtttttactcctcagaatttcccagaatgtgatctggttttgattttcaagcttgctgacccaataggttaacccacaagttttacgaagaccatctcagtccacttacatcaactgcccatgccacggttaaagagatcatcgactgatgtttggcacagcttcctccctcttgggtgggcaagcatttggaagagaaggctcctatgggtgagagtggggcaccaaagtcttccctgtcccatcccctagcttgagaagcccttctctaatgtggactttgtgccgttagcatcgttactagcttgaagttgaccatctggacgtactttctggtttagcctcacaagtgagcaaggagggttgagagatgtgctgtgaggaatgtggggccccagctggcagcaggctctgggtcaggggggcagggaccacgggcatacctgacagtgaggaggggccacacctgcagaaaaggatgcaggactccgccttgggaagtgttctaggccagagcgagggtctgtggtttataagtacacccacagtgctcgggaccctgcagatgtccagggtgccgtctgagcccgtatcatccaacagaatgttctgctagtgaagattaaagatttactccaggggctttaggatttattatatatatataaatcctatatatataattttttttttttttttttttgagatggagtttcgctcttgttgcccaggctggagtgcaatggcgtgatcttggctcactgcaacctccgcctcccgggttcaaactattctcctgcctcagcctctcgagtagctgggattacaggcgcccaccaccacacccggctaatttttgtattttttagtagagacggagtttctccatgttggtcaggctggtcttgaactcctgacctcaggtgatctgcccgccttggcctcccaaagtgctgggattacaggcatgagccaccccacctggccaggatttattgtatttgaaccatctaccattttaattttgatgttatgtagtatttgatgataatgaaagttaaattgtttttctttccatttttctgtttaagtgaatgacctgtatctagtttattcagtaacttcctgcatatatttgtttctttcattcttaatgaatatattcttaatttagttgctattatgttttgctttgccccaaaattgaaatcttagtttccttttagctcgttttagaactagtgatgggatgtgtcttccataaatctcttgtgatttgttgtaggctttgatggattctaatcttccaaggttacagctcgagctctataaggaaattaaaaaggtgggccttgcttttcttttttaaaaatgttttaaattttaaatttttataggtacacgtattttgtaggtacatgtaaatgtatatatttatggggtacatgagatattttgatacaggtatacaatacataataatcacaccatggaaagttggatatccatgccctcaagcatttatcctttgtgttacaaacaatccagttacatgctttacttattttattttatttttgagacagagtcttgctttcacccatgctagagtacagtggcatgaccttggctcactgcaacctccgcctcccgggttcaaccgaactttgggctggtctcaaactcctgacctcaggtgatccgcccgcctcggcctcccaaagtgttgggattacaggcgtgagccactgtgccgggcctgattgtacattttaaaataactaaaacagtcagggcacagtggctcatgcctgtaatcccagcattttgggaggctgaggcaggtgatcacctgagatcaggagttcgagaccagcctggccaacatggagaaaccctgtctctactaaaaatacaaaaattagccaagtgtggtggcgggcgcctgtaatcctggctactcgggaggctgaggtaggggaatcgcttgaacctgggggtggaggttgcagtgagccgagatcacgccactgcattccagcctgagcgacagagtgagactttgtctcaaaaaataaaaatgaaataaaattgggccgggtgtggtggctcacaccttagtcccagcactttgggaacctgaggcaggtggatgcttgagaccaggagtttgagaccagcatgggcaacatggcaaaacgctgtctgtacagaaattagctgggtgtggtggtgcacaactatagtctcagctacttgggagattgaggtgggaggattaattgagcctggaaggttgaatctataggtagctgagattgtgccactgcccttcagcctgggcgaccaagtgagaccctgtctcaaaagaaaaacaaaaaaacaaaaaacaaaccactattatcgactatatattattgtctatgatccctctgctgtgctgtcgaataccaggtcttgggcccttatttccatcactgagcaaacttcactctgttaagcagcaggtgtgggatttcatcgttattcagtaattcacaatgttagaaggaaatgctgtttggtagacgattgctttacttttcttcaaaaggttactctttattagatgagatgagaattaaaaatggtaacttactttatatctttataattgaagcccactagaccttaaagtagttaccagatgttttatgcatttaaatggccttttctctaaaattagaaagtaacaaggaaagaaaatgcttcgtttctatgcaaccctcttggtgactagtatgtgactcttaatgcaaccctcattgcaccccctcagaatggtgcccctcggagtttgcgtgctgccctgtggaggtttgctgagctggctcacctggttcggcctcagaaatgcaggtaagttgtacactctggatgttggtttttgtcgggggccagctgctactgatcctttatgtctcagctcagatgtcatttcaaaagtctgctctgccctctccaaattgcagtcgaccttgccctgtttatgtttccctcatagcactaatccatgtcagaaattgtcacgtacagtctatctgtgtgcttgtttattttctatcccacccttccgcaagagacttatgggatgtgtgccccaggacagcaggggtcttactgtcttatgctctgttgcagcccagcagcgataacagtgtctgcacatagtacttgcttaaaagatacttgccaaattgttgaaggttgaggtaccaatttcattattgctgactataggagttatagcaaaatatccatttgtctgttacatgagttaaaaatatggttgttgcactgtgaatagtttggtttagtcaaaacagttgtatcttaacggattgagaaacaaaagcaggaccacttttcatcagctccctccttctccttaaccagcaatacatgctgatgctgatatcccatagaccctcagctccatcctgagtcactgggaatgtggtctaaaccctcactattaatatgaactgagtttcaataagaatcttatatgggtcgggcatagtggctcatacctttgatcccagcacttcaggaggccaaggcaggtggattgcttgacccagactaggcaacatggtgaaacgccgcctctacaaaaaatacaaaacttagccaggcatggtggtgcgtgcctgtggtcacagccactcgagaggctgaggtgggaggatcacttgagcctgggaggtggaggtcgtgttgagccaagatcgcaccactgcactccagcctgggcaacagagtgagacctgtctcaaaaaaaccaaaatccagaaaagaacttatatggctgcagaggtataatcactaaggaaatttccttttgtataatcttttttcttttactatcatttaaaaaaatgtgttatatttctgaagcaacacatccaggttctgcacatagcagccaaagtgaccttaaagaatataactgggtcttgtcattcccttatttaaactcttgtacccatttcccagtgccgtttagatagagattccagactcgtcaatggctctgtcacctcagacaccctgcattgactcattagtctgattagagtcaggtttttcttcctcctgatggtttttttttcccccttagttctcagcggaacagtcacttccttagggaggtttccccagccaccctctgaggccgtgcttgttgccagactctgccactagagggcagggctgcaccactcctggcacctcgcacccggcctgccctgtcactctgtgtgttgggtgaattcctgtgatctgtgactcactgctctgtgtcctacacattcggcttttcttctctccccacaaccccattttataattctcctttttcaggaaagctttattcccatttaaaaatttttgtttttaaaatggtattttcttacacttattttctaattaaaaatgagtgttttaagaagtattatgatttactgcaaataatttttaaacccagccttttagatcctctgtgatcataagagaaatgaaggatgtctcccaacacttgagcttcatccacatttcatcctcctgttctttcagctgagttttccccatcccattagggactgttggaatataaaactggcttttccctaacagggaatgaattgcttctgtttctcctgaaggagagctggaagaatgacttgcgttcttttgcatacacaggccttacctggtgaaccttctgccgtgcctgactcgaacaagcaagagacccgaagaatcagtccaggagaccttggctgcagctgttcccaaaattatggcttcttttggcaattttgcaaatgacaatgaaattaaggtatgattgttgcctcaggtcacaaacatgcgagtgatgctgtgagtgagtctgtggagggtgagggcttctgaacagggagtcctgtgggagtgcttcttggggtatgttgtatgtcgtaatttagactaccatcatttgtgttatttttgaggcacctaaggacttctttccacttctcatttcttactgtggggtgaagagttgaattgggagatggtttctagatgcaaattgaaaaggcatttttccagagcagatttgttttcggcgtactagagtgactctttaacctagctgcgggaagatgactgtgccaagactgcaggtaggagaaagctcactgacgaggccttgtgggtctgaacgtcctgcagctatcagagcctgttggcttcctgttgtgcattccaacaaatcatcttcaaacccactttagtgttttgtttataatgtccagaaatagtgaccctgtcacatgctctacagattacaggattcttagcctcttcctttttggtaggtcagtcctgggtttgagcccaagtgaccctcctgggaggtgatgatacacactgggtagagtggaatcagatggacttggattagaattctgtcctctttactagttattttcctctaggcaaactgcccaacagctctaagctatttccttcgtattctgaaaaataagccttaatgggacccatatagggcaactctgagagtaaaataaaggaatatgtgttagagtgtagcatagtcacccacgggaagggcttagatgttagctgctactgctcttattagctgaatgatttggaataaactgttagcctctctcatgttttttctcttgagcttcgaagttttcttgttaatactaaggagatattcaaactagtcatggggttttggaatgacgaagggagatgatgaatctaaagaatttagtgtaatatttcttcatgctcagtaaatggtagtttctgctgctgttatttttattaccatctctttggaatgggagtaggtgctcctttgtggtcagaggctgtgagagctccacagcgccagtttgcccatctgtacactggggtctgttgaaggcagtcccctctgtgatatctctggctgtcagagctcagatgatagatggtatttttgtactcttagttctcatcattttcatgatttcgatcaccatttgagtatgatgatgctaacactttgttgaacgtagaatccgttaattacttccttcctgaacctttggcattaaaaaaaatctattctgctacctctctgctcatttatggttattcaaatttattatcaagagcctggtacagtggcttgtgcctataattgtagctacttgggaggctgaggtaggaggattgcttgaggccaggagtttgagaccagcctgggcaagatagtgagaccctatctctaaaaaaactgaaaaaaaattagctggacatgatggcatgtgcctgtggtcctagctactcaggaggctgagacaggaggctcggttgagcccaggagttggagttcgaggctacactgagctgtgattgtgccaccacactccagcatgggtggtaaaacaagatgccatttcttaaaaaaaaaaaatatatatatatatattatcaatgaaattcagtagtaccaacaggattataaacaaagatagtagttcccttcctactttttctcttaatccttgtgtctcacaggcaaacataactcttagtatttcttccaatatttactttcatgtttctttctttctttctttttttttctttgagatggagttttgctcttgttgccaaggctggagtgcaatgacgcaatcttggctcaccacaacctctgtctcccgggttcaagcgattctcctgcctcagcctcctagtagctgggattacaggcatgcatcaccacgctcggctaattttgtacttttagtagagatggggtttctccgggttggtcaggctggtctcgaactcctgacctcaggtgatcctcccacctcagcctcccaaagtgctgggattacaggcgtgagccactgcgcccagcaacttccacatttctaaataacatgcttctactgctattttttttttcaattttagacatttttttactttcactatagttctatcagaattcagtgtgtacgttattatgcctaagtaaatagtcatggttgcttacgtattatatttctttgattgtgtttcttatttgatgagaaagctgtgttttttgctctgggttgaaactggagagaggacctggggaggaggaggaggacagatgaagttggtgactgtaccttcatggccatagctgggttctcagcacccggggatctgctgatcacctactcataggccaggcccctatcgaagttctaggtgacccagtgctggggacgggggggccacctgcaaggtctaatcatggaggtgggggctacagtgttggcttgtgctggggccagcatccttaggaaggcatcttggaggtggaggagacagccgcccacttcttgattggggccttcagcagcaccagcttcttgggcaggctggtgctggctttcatcaccatgtcgtgttcaatcttcttccagatcctgacttctaggttcagctttcctcagaccctggttcctttcagaggccattgctgctgccttgctctttgctggcttgtgccttgattatatgtctttgtacaactttttgttttcctggagttaatcttcacatctgttttcttggagttaatcgttacctctatatcgcttgcttattattctttggcctttttgtcttctcacaccttccaacttctttgtaatatgtgtttagtacaatttttcatgacaggtagtttactgaatcagtttttccccagtgtggtcatccaacttgagttatccagctctctgccccagtctgggcaggttgatcttcaggtctgtagtacacttgtatcctaggacttctctttgccattagcctggaatttcctttgcagttctcccgttggatgcccagttcctagatgccatatgtttttctatcgtctagtagcttcctgagagaagatgaatgggagggaaattgtatgaggttttgcattcataaaaatgccattttttttcctgtacacttggctgggtatggtgttctggggtagaaatcattttccctcagaaatgcaaagtctttgccctgttgtcttaaaatctccaacgtgacccgattccttaacctatgaatgtacttttctttggaagctttccatttttggggaggtgaagtgctaggtacttagtaggccttttaatttggaaacttacatcccttcagttctgggaaaattttcttaacatttctctgagaagttcttgccttttattttctgtgttctctcctgaaattggttagttggatgttggtcctcctagattgactcacatcttacctttttcttttctttttctggtactttttagatatccatctcaaactcttctattcattgttatgtttttaacttctttcttttctttgtctcttgatggggtcttgccctgttgcccaggttgtggtgcagtggtgcgatcatagctcactgcagcctcaaattcctgggctcaagcagctgttctgcctcaccctcccaagtagttgggactacaggtatgcaccaccacgtccagctattttctttacttttttttttttttttttgagatggagtcctactctgtcgcccaggctagagtgcggtggtgggattttggctcacttaagcctctgcctcccaggttcaagcagttctcctgcctcagcctctcaagtagctgggattacaggtgtgcaccaccatgcccggctaatttttgtatttttagtagagccagagtttcaccatgttggccaggctggtctcgaacgcctgacctcaggtgatccgcctgccttggcctccgaaagtgccgggattacaggcgtgagcccatcattagatctttaaataccagtatctataagtcttttcctcttgagtcagctagtatccctggaaggaaattactcattttcctgcttggaggctataagcttggctatgtttatcctgcaaccggggactggaagggaggggactgacagtgttgctggtcagggtgccctcttactttttgttttctgtgtgcatctcacgtctgtcctcagcctatgtaaacacctcttgagattatccctctcaatctttgccggaggtgggggaggggctgcttcctgggctgccttggattggagggaagacctcaggtgagtgggtgggaatttgcccaaggagccatgagaccagccactatttcaccctctccatccctccactttcagatgtatgtggcgcctccaaagcccgagctcttcttggcgtctgtggcttcaataagcttgctttttgctggtatccctcctaccctcccctgtccccagcaaagcttgcatttgaacttcttcctacgggctaacaaatcagtcagttatgtagctcttgttactttttagcttccgaagttttgttgacacccgtagtctgctaatgtccctgttctgttctttctgttcgtgtaaatatatgctttatacaacttctttacatgatttttgtggggtttctgggtagcagagcttcacaagttcaatccagcgtgttggattagaaatctcccaccctctggtttattcttattctcaaaattacctgccaaacactgatactcccttgtttttccttttcctgacaggaaatgtacataccatacaggacagaaatcattagtgtatcccttggtgaataaccacaaagtgaacttaacccttgtaaccgccacccaggtcaagacagaatattaccaagcactcagaagcctctcccctattcccccgtcactgctcctgccttcctccccaaggtcatgactgctggcttctaattccagagtctgtttttaaattctgtgtacatagaccatggattaagtgttctttttgtctggtttattttggtcgacattaagttcatgagagtcttctatattatcgtgtgtattagtattcctgtagttttaggagcttcatagcattccattgtagggatataccacagtttattcattgtattatcactgggttgtttctagttcttggctattgcgagcagtgctactgtgaccactcttaggtgtgtcttttggagtacatgtgcaggtttccatcttgcacagctagaggtggagttgttgggtgatagggtgtgtgcatctcagctgcagtagaaactgccaaatagctttccttgagtgcttgtaccagctcacccttttgccactgtgtatggggattccaggagctctggtcctcgctagcacttggaattgctgatgcttttactcttagccttcctgatgggtgttttctggaatcacattatgattttaatttccattccttaaagtacccttggctctgaagtttaatgattcatgcatctcttcccttttgaagtactcttacaggtatgttgtgcatgtgttgaaaagtggcactatctattctaaaatacagtatgcctcctctgtgtttgaacagttgtagcgtggccttggggcctcctgttagctggcttggagaagggattcttgggattgtagagattagacctgaggaggccccttggagctctctgactaaattttattctttattattccaaactatttaagctcaccgtgtgctgactcatcataataatgagtagctctcattgtgcttgtctatttggactcatacaatgattttttttttttctttgagacagagtcttgctctgttgcctaggctggagtgcagtggcacaatctcggctcactgcagcctccacctcccaggttcaagtgattcttgtgcctcagcttctcaagtagctgagactgcaggtgcgtaccaccatgcctggctaatgtttgtatttttagtagagacggggtttcaccatgttggccaggttggtctcaaactcctgacctcaagtgatctgccttcttcagcctcccaaagtgctgggattacaggtgtgagccactgagcttggccaaagtagttttttaagatgttagtatcttttcttgcagctaaaaaagtttgtcagagatgattctactttgttctccaggtgttttctcagggagaaattggaggcagtaagccactgggggagtcctgtggctggggggtggggtagtcctgtggctccttgtcagggagtcctgtggctggcaaggagagaagtcctgtggctgggttgggagggagtcctgtggctggggtctcatcctgtgcctaacagtgtccagaggtgccgagaccagctcagtcggggagaccctaacccagcagcgctagaggaattaaagacacacacacagaaatatagaggtgtgaagtgggaaatcaggggtctcacagcctttagagctgagagccctgaacagagatttacccacatatttattaatagcaaaccagtcattagcattgtttctatagatgttaaattaactaaaagtatcccttatgggaaacgaggggatgggccgaattaaaagaagaggttgggctagttaaccgcagcaggagcatgtccttaaggcacagatcgctcatgctattgtttgtggcttaagaatgcctttaagcggttttccaccctgggtgggccaggtgttccttgccctcattcctgtcaacccacaaccttccagtgtgggcattagggccattatgaacatgttacagtgcttcagagattttgtttatggccagttttggggccagtttatggccagattttggggggcctgctcccaatacagaggtctcgtgtaaattccctgggaggcgataagcctctgagaaacagactatgctaaccacgccatgaaagagaaacttatttataaatcagatgccagttactagtttactgcttatttgcccaggcgtagctctgacagagtccccgactcatagtgcttgctcagtgcatgctgaacaatgattggaatcaagtcatggctcagagcatagttttgaataatgggaaatggatgttcttaagtaacatagtcaccaagataatgcgactagctgggtcaccccttttcaattttaggatatttttatcaagatttaaatggccatcattagagttatagcactttctcctttggattgtcctagaggcccatgagaaagtattccctaatttcttaggagaacagtttgtgggtagtatgcggtcatgtccagttaaattgcagatatttccgatcgaagatgttccagtcctgagaacttcgtgacattagcaggacttctacaagccatctcttagggtggggcatttactgcagttggctagtactcttttctccttaactttgtcatttgttgatttttttttaactgtccccaaatactgtgggcagagtgtatctagaattgaggcctccaccattgcggagaggacatggatgctgagcagtcccctgagtgaaggttataaagaagcaaatagactacacatgtctgtaaactgctcttgagtgtcccaaatttggggtacttcagttcagctgtaggaaaagcctcaaactgtttatactttgcaagaattggaaacttctaattcacgttaagttttatgtaatacatgataagcttcataggagcttcatcttttatctacttggacttttgcttccgtaggttttgttaaaggccttcatagcgaacctgaagtcaagctcccccaccattcggcggacagcggctggatcagcagtgagcatctgccagcactcaagaaggacacaatatttctatagttggctactaaatgtgctcttaggtaaggtggaggcatatgagtggaagagtctccagcatgtactcaagatagacctttgaaataaataaaaccagatgatccctcagcttctagaccaggctatttggcactggttgattgaatgtgaactgcactggggctgctgtgagcccgcatgggtctctgtgaccctgcagatgcagccgtgcccagggactgggcagtgggtgtgggctggtgtgagccctgtctgccacccagggcctggccctctgtctgtgtcggccatgactatggtgagtcttgtaggcttgagactgtgcctcgggttcctgcgggttctctgtaggtcagttgacagtttctcctgttgtttgggtaactgtggaaacgaacactggcaagtgctgaagcgagcatgtggacgtgcgatatgaaataacgacctggctttcaaaggcagtgaggctctctggaaaggaccttgctgagctagggatgtgggtgtgtagccattcccagtgggcctcatggcgtactcgttcatgatcatgtttgtgccatcttgatctctcaggatctcttcttttttaacagattaagccgggaatctccaaacagtgagtcagatgttaagatgtcttgcttccacccccacaggcttactcgttcctgtcgaggatgaacactccactctgctgattcttggcgtgctgctcaccctgaggtatttggtgcccttgctgcagcagcaggtcaaggacacaagcctgaaaggcagcttcggagtgacaaggaaagaaatggaagtctctccttctgcagagcagcttgtccaggtaggagcacagggtttactctaggccctgcatgtgaatgactgacattcaaagaaccgattaatttggaagagaagcggcagaaccgagagttagaggtgtggactctggagctgcgctgctcgtttccaaccctaggtgctgacctctagctgtcttccctctgtatgtccctgtcaccgtgagtcaaatgcgggtgatgcctcctcaggtgccgtgttacctaagcctctcagagaccactgctaccctgtttctaaaaccagaggtcacgatatgtgttcatccacccagtaaatactgattgagcacccactgtgtgctaggctctgggataggggctgggtatacaatggtgagtatttcagctgcagcttctgccccgtggaggctgtggcctagcacactggtctaggcacggtggtatatgctcactcaaggagatagggacgtggtcgtttggggtgtcggaacaaaatgtcggaacttctctttccaatgcagagaaaccttgcagtaattctaatgtactgtgattggcagttgacttcagttctttgtagcacgcttactcaggttatttcactaactatgtaaccatgcagcctcattttaagcaattggattttttgaactttacttaaaatgttatgtcagggtttttattgtgcttaatgtgtgccatttagctaagttttgtaggatacgaaattgtaagtggcttaaaatgattcttaatagaatcatgaattgaagataatgctaataatttaagcactgagttaggtagtgtttgtaaaatgcttagaatgcttcctggcacatgttaaggccatgtaagtgctgcgtgttgataaacagctgagcaaaagtggactcttaagaaagtattggggctgagagttctgttccaaccagctgccctttggttatttttcagaataaaagcagagtctcatgggatatgacatttatatttccttcacaaaaaacactgctgagtgttttgttgagtaaaaagggtgtagccatggtaataatacatttaaaatatagtttatttcatctttaccttgccttgttttttttttaagctagctttttattgagaattccacacatacaaaagtatcaactcatgaccagttatatttcatttataatcctacttctccctttttttattatttgaaagcaaaccccaattatcctcttatttcatctataagtatttcagtatctctatagatgaggactcttctttatttttaaaactttatttttaaaatgatggtcagatgcagtgttcatgcctgtaatcccagaactttgggaggccaagctgggcggatcacttgaacctgggagtttgagaccagcccgggaaacatggcgaaaccccatgtcttaaagaaaaaaatcagccaagtgtggtgatgcatgcctgtagtcccagctacttgggaggctgagatgggagggtcacatgagcctggaagatcaaggctgcagtgatccatgattgtaccactgcactccatcctgggtgatggagcaagattctgtctcaaaaaaacaaaactgcaaaacaacgtcacaaaacagtgccattgttagacctgaaaatattaaacatttcctacatcaaatacccaccaactcattatcaatttttctctctactcttttggaatcagcatctaaataaaattggtcgataaggattgtaaatctctttgatgaactggttcccctccatcccagtttttttcccttagagttcatttattgagaaaccagattgtttgtcttctaagttttcctgtggtctgatatactgcttccatctccactgtgtaaattaacacctttttctcttctctgtatttcctgtaaatcaataattggaggaaaagccttgtcagatttagtgtatattttatatctgagtccagtatttcttatataatattttaagataagtgtactcttttaaaaagtattgaaactatatgctcaattttttttaactgatgcttttaagaaggctgcttgatcataaaagtttagagatcattggtctgatgggaaaagcaaataattactaaaccgtttagcaaggttgaggtgcacatggtggggcctggagaagttcagtcatgagccgtcacttatgggcacgtggaatctgacccggcacagagttgggagaagacaggagctttatagacagaaaatgtggtctttgctaagtcccaggagtgaaagggtgagacagtgctcacagcacacgagtgtgggtgcgtagacagagcaagggtgggtcctgaaaaggcctgcaggctttctcatagattagcaagagtgctggttacggaggtttctaacatttgtgaacagatcgaaactgtgttaaattgggattgcagtaatcctggaaggacagggatagagggtgaaggggaaaaaagggtatggatgtgagacttaattgctgattttcttaagacctttctccaaagtaaataaatgatgtggcacatttttgaactggcaaattctaaactctagatatgattatctctataacatatcttactccatcttcttttgactaaaaactgttcttaattaaattaccatgagacgttcaattcagcaaatgtagtttggctaaccatatttaattagaatttaatataatcctaggcctggccaaactattaagcaagtgtgggcaaaatattgataattttagatatgcaggaacttagtttgctttccatgtgtgcttttcgaaaaaggaataaattgaaaaatagaggaagccctgaaatccaagaagcaaactctctcacctaggcatgcagtaaaagcaattctaggatgattgctgtttggcgcgtagttcgtattagaaaccattcttcttgaataaatagtatgtttaagaagctgggcagagggaaggcatatgcatatattatcaacaaggagggagaaaaaggcaattagtaaccatccataggagggtcagcaagatttataaaggaaatttgtgatccaagtatgaagcaaaataaggtgcagaataaattttaagcaagtaatagattagagtaagagaacccatttgaccattaaccttgggacattctctttcaaatgacatggagtagtactgaaatctttctttctttctgagtctaggttattgtgactggactcagaaagaaatatttcattattgcagtgaataacatttgtgaacattattgttcataaattatgcagtgaataacatttatgaacacgtgatgtgtaagatacatactgtttatttttagttaagttttttggctcaacttctaggcagagaacattaaatgtaaatagtgttacctaggagcatgtaaatggaaatctccatagtatgaaagcagtgctgttgctaacagaatttaggagggggcagatgaggtgaaggaaatgtgggtgctgatttccttattacattgagaggagccaggagattctttgttcaaaatggatggcttaagaagtcaaagtataagctgattacgtagagcaggtacccaaaaatgttttgtgtaaggggccagatagtaaatattttcagtcttgcaggccatcccaagtctgtggcagctactcaacactacctttgtagcatgaaagcagccacaggcagcccataaatgtggctctgttccggtgaaactttaggtacaaaagcaggtgcaggccagacctgacctgtgcactgtggtttgctgacctgggattcaggggtatagaagttaccatcagaagagctaaaagtgagactttttactttatactcttctacactgtctgattttgaaaaaaagaaacatgtattttataatattaaagatagggttggcaaatagcaaataaaaatacagaataccagtgaaatttgaacttcagatacattatgagtaattttatggtgtaagtatattccaaatcatgtgggacatacttacactacaaaattatttgttgtttgtttacagtttaaatttgagtgccttgtattttatctggcaactgtaattaaagggaaaaagaataaattcattatgttcatataatgtgatatagcaggggtccccaacccccaggctgcagagtggtactggtccatgggtccccaacccccaggctgcagagcggtattggtccatggcctgttaggaaccaggctgcccagcaggaagtgagcagcaggtgagctggcattcccacctgagcaccgcctcctgtcagatcagtggcagcattagattcccataggagtgcaaaccctattgtgaactgcacatgtgaggggtctaggttgtgcgctccttatgagaatctaatgcctgatgatctgaggtggaacagtctcgtcttgaaaccatcccctggccctgtggaaaaattgtctcccatgaaaccagtctctggtgccagaaaggttgggtagcactgtgatatagtattaaaagtgctaataaatatggcatactgcctttaaaatgtctggtagctctttctcagtggcactcataatagtgttttttgatttttaaatgtgtgtcaagctgactctcccctccgtgtatgctgggctttattttccctttcctagtcaccagttttgggaaatagagatcttcattctcatgctgctcctctagtgcaagtgctccatttatttttaaggaattaatataacaaaaaatcatgggaatttagaaaacaacatggaagctaatgatcacattggtggaagtgatagggaaatatttagggggagaagttaaggtataaactttgtcaatgaagtcctattaaaaacaacaaaaaagtgaagcttaggatgcattttataaactctgaccagaacacctgtgtttctctgtttctaggtttatgaactgacgttacatcatacacagcaccaagaccacaatgttgtgaccggagccctggagctgttgcagcagctcttcagaacgcctccacccgagcttctgcaaaccctgaccgcagtcgggggcattgggcagctcaccgctgctaaggaggagtctggtggccgaagccgtagtgggagtattgtggaacttataggcaagttattagcaaggtctactcttacaattaactttgcagtaatactagttacactctattgattatgggcctgccctgtgctaagcagtctgcattccatcttccttgccaaaacttataatacaaatttcatctttattttataaataggggagttgggctgggtgtggtggctcacgcctgtaatttcagcactttggaaggatcgcttcagcccaggagtttgagacaacctggccaagtgagaccctgtctctacaaaaaaaaaaaaaaaaaaaaaattagctgggcatggtggcacatgcctgtagtcccagctgctttggaggctgaggtggtaggattgcttaagcccaagaggttgaggctgcagtgaatcttgatggcagctgcactgagcctggtgacagagcaagatgctgtctcaaaataaatttaaaaataaaataagagaattaaagtttagcaggttgggtggcaaaatgaggccacacatttaaagcccctcctcctgattcttttctctgccttggctgcctcctgtggcattttaggtgctgagaaatgaaaacagtagggaaaatagttccaggatcctcatgttaatttgccagaaatggcatcttcaagtcgtcagagggatctgagagttccttcctggcctgacttgagaaaatccgtctgtccccagctctgcgtctgcctccactgcccagtcacctcctctccatgctcttggggctgggccctaccccaccatgcagtgctgccctggagcagtgagcttggtgggtcctgtctggcatgagagctgcctttgggagctggatcccagcctctaccactgggtctggtgcctagcaggctatggataaacttctgctgactccggcctctcctaagccactgcaacgtggtcggtgtagtgcacagtgtgtgtgcagcgtggccttactcacagcctccacattagagagaatctgactgaagtcttactgctgcctcgtgtgaacataaatgtttgccagaaccatgagcaggaaatgttaatctgccttgtttcctgtcctttacacggaagaatttttttctgtatggaatgcgtgccttacaaataatgagtggaaatacccatcgctaatgaaaagttatacttgactgttagtcagctaaataatctgagatttctaatacttttaatttggcttttacaatgcaatttatcttagcttttttgatttcttaggtcatatctttagaactatatatttgaatgttaatgtaattttcatattgaaattaaaatgttgaactgcgatgttaagtgtttcctgtggaaaaacgttcacattttctctagttttaaagttgaatcaagctgtttgaagattttcacatttcttctagattttatcagcttgttactttatctgtcactttctgtgatttgcagctggagggggttcctcatgcagccctgtcctttcaagaaaacaaaaaggtgattatttcagaaatcagagtcttgtgttgaatcttactgattttcttgtatttctgtaatgtaatgtatcttgtatttcttgtaatactgtattggactctgtgtatatctcttctcagatgagtgattatatgtgtgaatgttgctggaatctgataaccaggcctgaatagttttgtagggtggcttttaaaaattactttcatatcagaattgctttgtcataaattttgaacgcatcataaatttctaatgttcggggtcagcagactttttttgtaaagggacagagtgtaaacatcttagctttatgggccatatggtctcttttgcaacattcagctctgccctgtgacaggaatgcagttgtaaagacatgagctactggccagctatgttccagtagaactttacttacagaaacagacaggctgtagtttgccaatacctgccttagggaatgtgttgttatattttgtgagttaccttctcagtaaattttatttagtattagtcaggaatattattaagtagcttcttttccagcctggtcaacatagtgagacccggtctctaccaaaacaaaacaaaacaaaaaaacagccacgcatgtggcatgtgcctgtagcctcagctgctgctcagggggctgaggcaagaggattgtttgagcccaggagtttgaggtcacagtgagctgtagtcatgccactgcactccagcctaggcaacagaatgagaccttgtgtcttaaaaaaaaaaagtttcctttgttgggttattttaatttggacctggttatcatttttcagccatatttaactttgtacatatcagaatgttctgataaaacttaacttttattaaagtgtttgtgatataatctgctagttttggtacacattatcttttgcaatgccagttattttcttttccagtgtgggtttgcataggaaaagaattgctgtcactttctattttgaaatcttaaaagactgatccttttttgtgtcatgatttgagtatttaattgagagcctaatgcctaatattatttgcagtattaaatgggatcttaacaggaatagcattctagccttcattgaattaagtaaacatttcttaagagaacttggaatctataatatttgcgtcatcatagtatgagatacttaatcaagtttgagattttagtgaaacattgtttagaagccaaaaggattctaggaaaaattaatgtctatattcttgaattaggagagattttgggacgtgtgactaagttacgctgacacttgtttgtttcttagtcgctttttccagtggcggtgagaacgaagatgactgattcacattgctcagatgagtttatcctcttctggctgggacatgggatatatcctgtctcttttaagcctttttggtatttttcccccattgagagctgtgtcttcaaactcttctgttatagctggaaaatcctttttaagtgaaatctgcccaaattataagacagatgaaggtagagttgtgttggatataggattagggtgaaagtagtgggggtgtcctggagcctctcttctggtggcagcctagctcttgtgcctttgaggaaattaccctggggacggctctgtggaacatatttgcaaaccactgatttggaagatagagatggcttttgttaagatctgaattcacctttttggcattttatttgatttctcaaggtaaagaacttattttgtaataaagtttcctattatttagtagataggccaagttgctgtgttaattccatgtagattttgggtttcctttgctcattttttcactcttaatctcacatcattgtaagtttatggaagttatcatacttctgactttttctttgaagagcagaaattagaaattcccaataattattttgatagtgtcatttaatgacactcacatgtgatgtagccacaaagatttaatgagttcagttttaaatcatattaagactgttggtttcatttgttctcattaatgtaattctgaagatgaacaataaaatgtatttttagaactttcaaatgaaatattatttcatccttccagatcatataatgcttaagttctgattgttaatcataaagtctagaaaattaaaagataataaaatgaaagtgacttttaggtattagagttttattataaattctggtgtgtcattggagctatgacatgaatatttcaaaggccaatagcattggatctttacagttataacttaccatttttaagtttaagtagtaatatagattatttaataatcaaaatcaataaatattaattattaaaatgttttgtggtatagtttgagaatcattgcttttaactttttccatataggtttattgactttaatagcattctaaacataacatctctacattctttgtgtttaatactgtggaggtataaaaatacttatatatgatgataaactatattagagtaaattaaatattcttatgagtttcattttagagtgcatttacttaattttgaagtccttatttttagcaaactaaaaggaatgttggtacattatttactaggcaaagtgctcttaggagaagaagaagccttggaggatgactctgaatcgagatcggatgtcagcagctctgccttaacaggtagttctcactagttagccgctggtgtggaccttcactgtctgccttccaccccttgcccttcctgctcgtccccctgcacctggtggacagcacgactgggggcagcagtggagccaggttgcttaaatggggcatattcgggcttcttttataatacttactctgaagcttgtgtgtctgtggtgtttgcatcatatatttgttgttttccatggtttaggctgttttaaaattaggtttatggcttgagcatagggctttgtgagtaggggatggcaggtcgaaacatctcatgagttggatgggttatgctgggggttgggaaatgggatgaaaaattatgggatgaaaaattgcctatggatagtttaacttgaaagaatctgcctttgtttacagatagttatcttttttcttttttgagatagagtctcacactgtcacccagtgcagatacccagtgtcactggagtgcagtggtgtgctcttggtgcactgcagcctccgccttctgggttccagcgattctcctgcctcagcctcccaagtagctgggactacaggtgcccgccaccacgcttggctaatttttgtatttttttgtggagacgggtttttgccatgttggtcaggctggtcttgaactcctgacctcaagtgatctgcctgcctcagcctcccacagtgccgggattacaggagtgagccactgtgcccggccagttacagatacttatctaatgaaattctctgtgtactttataaaagatgaggattaactgaaggtactaataactggattatatgagggtggttttggttgtataatcctatctaaaagaatattttagctataactgaaagtaagacttaaatatttagagaggaaaatctgaataattctagtagtaattatttatttacaaaataaaaatagatttttttttgattacacaaattaaacaacaataaaacatcacagcaatccggatactataaagctcacatgcttaccgacccaactgccccaggagtgaccactgccaacagcttcatgtcgacctttttgccataatttttatatagccttttttgtttttaaatggtaatttagaaagtcaactaggaaaatgtgttacaggtttatcttccaggagaataggactggagtcgagatcttgaatgtggcttggaagaaggcaagcccaccccagagagatgagttgacagttgtttctgaccactgcttgcttagagggcctgcgtgtctgtgaccgcctagctttgcgcccctgactaggctgccccttaattacaaatgtctttatatattgctccagctaaggcttggagtagtcggttaagaacttgaacttcggtttttgcagtgaaacagcatttgagaatatcaccttctgataagccttattttataaggtgggtactgtagtgggaggcagtgtgagagatgcttgaaggatgcactgctgtcctgcatttcagcatcttcaggatgctgtgcagctgaaacatttgataacggtggaactgttcgttattttgcaagcctgtgattccctattgaatgttttctctcgccatttgacaaatgagtgtttctctgtcttcagcctcagtgaaggatgagatcagtggagagctggctgcttcttcaggggtttccactccagggtcagcaggtcatgacatcatcacagaacagccacggtcacagcacacactgcaggcggactcagtggatctggccagctgtgacttgacaagctctgccactgatggggatgaggaggatatcttgagccacagctccagccaggtcagcgccgtcccatctgaccctgccatggacctgaatgatgggacccaggcctcgtcgcccatcagcgacagctcccagaccaccaccgaagggcctgattcagctgttaccccttcagacagttctgaaattgtaagtgggcagaggggcctgacatctttttttttattttttatttgagacagagtctcactccatagtgcagtggaggccgggcacaggggctcatgcctgtaatcccagcactttgggagactgaggcaggcggatcacttgaggtcaggagttcgagaccagcctggccaacatggtgaaaccctgtctctactaaaaatacaaaaattagttgggcgtggtggcacatgtctgtagtcccagctgttagggaggctgaggcaggagaattgcttgagcctgggaggcagaggttgcaatgagccgagatcgtgacactgcactccagcccgggcaacagagcaagactccatttcaaaaaaaataaaaaaataaagtgcagtggctcgttctcagcccactgcaacttctgcctcccaggctcgagcgattctcccgcctcagcctcctgagtaggtgggattacaggtgggcaccaccacactcagctaatgtttgtattttcagtagagacagggtttcaccatgttggccaggctggtctcaaactcctgaccttagatgatccacccaccttggcctcctaaagtattgggattatagttgtgagccaccatgcccggccctgccacctgccatcttttgagttcttccctggagacctagacctgaaccctcctgcttgttctcttgttatctaatacccctattgacagcgcagcttagatcattaatggagagcttgacctcatctgataccttcactgaaggaaacaacttagtgtcttttgtgttgaacactgaggtaaaaaattggaatagttgattatatgaactctgctaaaattgagtgcattttacattttttaaggccttgttgggccctggttaaataattatttttaaaaatccttaaggagcctattataaacagatctgtggtcttaatgaaatgtgattaatactgtgcattattttaagaacttttgacttttcaaaaaacttttacaacatttcccatttgatagcggcataggtttaagcacttctcatctctaagttagtggacaaaaaaccctcatggatagtctaataatgtttgctacaagtccatgttgagttttatactccattttattttcagttttaaaaactgtggttaaatatgtgtaacataaaatttatgttcttaaccattttttgcgtatacagttcgctggtattaaatacatttaaataatgtcatggaatcattgctaccacccatctctgtaaccttttgatcatgtaacactgaagctctgttcccattgaactctattcctcctttcccgccaagtccctggcaaccacgattcttctttctgtcttctgaatttgactactttgggttctcatatactttaggagtcacacagtatttgttttacttagcataatgtccccaaagctcatgcatgttgtagcctatgttagaacttcctaatgtttcaggccaaatactattccattgtatggataggccacattttgcttttccattcctctgtccatggacacttgtattgcttcatgttttagccattgtgaatcatgctgttatgaacgtgggtgtacagatagctcctggagactctgctttccatttttttggctaaatacccagaaatggagttgcttttacattccaattttaatttaaaacattcatatcattgagtgttttacttaatagtatagtagttaacaaacttaataaaatagtattttggtaataatttgctggtagtccattgttcagtttttttaggtaaattacacaggacatttcaagtggacatgaaacatcttgtgatgtggaatcatgccccaagctgatggctaaacatatgaaataccataccctaaatttagtagatttagtctttgcaatttaggagataacctgttatattgttaggtttttgtcgaaaagctttgtcctcatatttccaacttgctgtaaaatttgtttgtgaagacaaatatttttgtatgggttttttctttttcatattaaaaagaaatgtccacattggaatttttttggagtttttagagctaatagagcttttcataatgtagtgggaatgagtgatcagtaagctcttagcagtttccatgcgtgcatttctgtgccttgaaataaatgacagatgagtacatttgtgttctgtgtgtaaaatgtgctctttcctcattgcacttccatgttggagggcttgtctcttggtgatcacacttcaaaattctcacagccccccttgaaccgtttaggtgttagacggtaccgacaaccagtatttgggcctgcagattggacagccccaggatgaagatgaggaagccacaggtattcttcctgatgaagcctcggaggccttcaggaactcttccatgggtatgtggactacaggtgatgcgctacaaagtggtttgtattcagacctggacatcttaattatatctttgcttccaagaagaagtcctttgatactgttttctgagttctgaatagctgatgaaaatgaccaattgaggaataatcatactttttcttgatctaaatcttatacttttgagttatcttagcataaatgtataattgtattttaagtggaaatttgtcacttaatcttgatttctctgtttttaaagcccttcaacaggcacatttattgaaaaacatgagtcactgcaggcagccttctgacagcagtgttgataaatttgtgttgagagatgaagctactgaaccgggtgatcaagaaaacaaggtgagggacataggcttgagacgacttggtgtttctgagcttgtgtgaggatttaaaatcgccctggctactgtctactttattgctttcccatccctgggcctttaaatttcccctttaaataccagctcttcccaggcctgttgttttctgcctttccaggtactacccacagccttgagaattgcctgagttctgcctcctttgagagtgtgccccagacaaatctattctgtactgaatgtttccttgtctgatttcttggatcattcatttgatggttgcgtatggcctgcaacgtttcttgttttggttctactgaactgttctaaaagtctctcttcatattatctttttacatgtaaatgtaactgtcttcacttttaattcctcaaggacaaggaatagcgtttcacagttcgtcccatcaatcagaattatagcctttggcatctccctatctaccaggcccacttcctcttagatttgggcttccccaggctgttgcctttccccaagtagcttctgcttgtcctgtagaagacctttcatgctttgcttctgcagcagccgttcctgaatgcctagtgtcaactgccttcttaccacgcccaccctccctgcatgctgcatttatcccctgccacagccctgtgaccctgtgtcctgctgcctctgacttgtctgtttctgcttggccatggtctctgtgaggtcaggtgtgcatatgggcacaaaccagggcatctctttatccccagcacctggcttaagtgctgctctggaactatctgttgaatgaactaatgcatgaatgtattgttgagtatgagacaaacaagtgtcattgtctcctttctagccttgccgcatcaaaggtgacattggacagtccactgatgatgactctgcacctcttgtccattgtgtccgccttttatctgcttcgtttttgctaacagggggaaaaaatggtgagtacaaaaggggatgtgcacagttgaaggaaataactaggtttcagaggtcagcttggtggcctgtttttgccttgcgtgcagcagaggaagtagaatctgaggatgagtttggttttcactagccgaggggagggaggaaatgatgggagcaggtaggttattgggtctggttttgttcatttgaaaacaatctgttgtttgaggctgaaggtggcttgggtgatttcttggcagtgctggttccggacagggatgtgagggtcagcgtgaaggccctggccctcagctgtgtgggagcagctgtggccctccacccggaatctttcttcagcaaactctataaagttcctcttgacaccacggaataccctggtatgttaaaagttcacatcttattttctcagatttaatcattattgtaaaaactatttcagtattgactattttagttttagagcagtaagtgttttgagttcatttgggatatttgacctgcgttgtagctcttcagaaaacacatgaatagtgaagttctttgtttcatgggttccctttagatgaaacccatagaggagaaaagtagaaacctcagcacgtaagagccaacatatatacacatcggatttaaacctaaagcacaaattgtgcctggtcgcagtggcgctgagtcgcactcagccaggccaggcattcacactcagggtgagtgggaaccaggactggctgaggcagcagtggacccaagtctccatcgcgcccatgcttactatggagccttctcgttctctctttttctttgggtgagagggtacacttgtgtttttgaatttatatgaggtaagtgtgtaatagggttttttctaatcttttttaagtggaatctggaattttaatcagatttattatctgacaacctagaattataatccagaaagtctgtggtattgaggacatattggcaatatgatgaatctctaattcttaaatcctgaaacttttttttttttaatcacttagggttattatagtgaagtcatttctgaatttggatcttctcttcacacctctttttctctttcctgagaattaagcttttgtttcgagttagaaagttgatagtagggaattgttccatggctgagcaatttatctccacagaggaacagtatgtctcagacatcttgaactacatcgatcatggagacccacaggttcgaggagccactgccattctctgtgggaccctcatctgctccatcctcagcaggtcccgcttccacgtgggagattggatgggcaccattagaaccctcacaggtaacggccagtttttcagctgtgttttttctagttatgcttactaaggtttaagtttagatgatgatgtttgttgcttgttcttctggttaggaaatacattttctttggcggattgcattcctttgctgcggaaaacactgaaggatgagtcttctgttacttgcaagttagcttgtacagctgtgagggtgagcataatcttctgtggaaccatttcttcacttagtggacattttatcattgctacaattaaaattggagcttaataggaaatatttccatgcactctaaagctgtaaccagtaatacccaccatgtatccatctctcagctttagaaagaaaacgttgccagtaaagttaatgcttcataaacttcagtttaagttctaattctcagaatatttgtttgaaatagacctcttcctaaaggatatatttagaaataacctatcattaagtgtaaagtctgttgaatatgctgggcacggtgactcacacctgtaatctgaccactttgggaggccaaggtggaaggattgcttgagcccaggagttcaagactatgggcaacatagttgaccctgtccctacagaaaattaaaaaaaaaaaaaaaaaaagtagctgggtatggtggtgcatacctgtagtctcagctactcgggaagctgaggtggaggggggattgcttgagccccagagatcaaggctgcagtaaggcgtggttacaccactgccctctagcctgggcaacagagtgagactgtctcaaaaataatagtaataataatcagttgaattaaaaaaaaaaaaaaaaaaaccactgtgctaggcccatagtatggtaagagttaaagtgagccttagggattatttactcaacctctgtttctgtataaagtggaataggctcaattctttaagtgatagcatgttgaacctttccataccaactggctcataagtcacaactggccagtcaacaagagtaaaaattaactggtaaaaatcaaagcaaaaaacctacaattgtcaaatttgtgggataactcccccttttaaaatgtcatgcctgacagtaatttctctctagtttccaggttttcagtcagttgtgtcttttttgagcagaaggaagcatgctaagagctcaatcttgtggctagctgggggtctttgtgtcagccatgcatgtgatggtgcccctgggtgcttggggctgcaggggaggggtacagcagtaggggcctgttctgttctctcgtgctgtggagtacatagtgacatagtggggtggtccttggtgtaggtcccttgttcctacccctgggtctgagatttatttagaagtggtgttggggctgtgcggcaggcccctctgtaactgatcaatgtttgtgaagttgctgtttgagagttgaaaccatgacataagcagaaatggaaggaagaaagaaccagttatgtgaaagggacacatttacttttaagcttgtatttactgagataaagtattcttaatcaatgttcttgagaggtgtgggaaaaatgcaacatcctggttgcagttaaacccagaacattgtgtgttgaagagtgacggttctcaaaccgtcaagacgcgggtactgagtgggactaacctgctgtcctcttgccttggaccttgtgttccagaactgtgtcatgagtctctgcagcagcagctacagtgagttaggactgcagctgatcatcgatgtgctgactctgaggaacagttcctattggctggtgaggacagagcttctggaaacccttgcagagattgacttcaggtaagtgagtcacatccattagatttcatgaactaagctcaattgaaagttctgggatcacttgatgcaaggaatgatgttatcaagtaccctgtccatcagaaatccgagtggtttaggtagatgacagtgattttctcctcccagtggctttttgctgaactttgccctatgcttggaattttattttattttattatttatttagagacaagatcttgctctgtcgcccaggcttgaatgcagtagcacaatcatagctcactgaagctttgaactctaggactcaagtggtcctcctgcctcagcctcccgattagctaggagaataggtgtgtgccgtcacactggctaatattttttgtagaaatggggtcttgctatgttgcccaggctggtctcaaactcctgggcttgattgatcctccatcttggcctcccaaagtgctgggattacaggcatgagccactgtgcctggcctagaattttaaaatataagtagaagagtagatttttttttttggtagtcctcgtcatttaagtattctggatagtgggaataaaagagcttagaatttttcatctttgtcttaaacttttaaaaaaatgtagcttatattaattctgcttgtttaaaaagaatatactcttcattatactgaacctaggtaagacagctggtttatattttgttgcaattaaaaaacgtgagctgtggttgcagtgagccaagattgtggccattgcacttcagcctggcaacagagtgagacttggcctcaaaaaaaaaaaaataacatgagctgtgttggcactttcattttctaagagtagttttggctggagaagttttctttcagtactttcttttagaagggaaattttcctttataatttagggtttgttttttttttttccaagccaccttttatagagcccttgtgggttatttcatttaatccttagaatgtttataaatctgggcttgttctcggctccacccacagatagggacgctgagcgtgcatgagtgggcagcaagatagcaggttatggagggcccagctcaccccttctgtggcttgagccaattttatagggcacttacagagtcttttgaaatagtatttattttgaagaaaaagaaaaacagtttactgagtactgtcttattgagtctggaattgtgagaggaatgccacctctatttatttaaagccattggccttttttgttgttttgagtaagtgctgcccaaggtccttccagggcacctggatgagcctgctctggagcaagctggcggtaagtgtttactgagtaactaaatgatttcattgttaaatgtgctcttttgttaggctggtgagctttttggaggcaaaagcagaaaacttacacagaggggctcatcattatacaggggtaagcggtttatttttgtgagatgctgttttaccttcaagaaggtgaaagtgaggctttccttgtggaatttctctaaatgcattcgtcatgttttagatgtttatttcacagtttatatcatgaaagttataatcttgtcatatggatttaagtctagtaatgttgagttctttctcactagctttccaaaatatcttacctaaaatttagtcaaatacaagattatgtttatttttattatccttctctctaaagcttttaaaactgcaagaacgagtgctcaataatgttgtcatccatttgcttggagatgaagaccccagggtgcgacatgttgccgcagcatcactaattaggtatttaccaatattttatctcttttccttttttggttgaagtactaaaagatacgagaatggaaagagagggaagaattcaaaggatgtagagcagtattcctgaatctgagctcatttcagccattctattcttaaactataatgaaaaaaaaatccaaaaaagtctaaaattataattaaaaaaacaacaaaatactaactgtccattgtaaaaagtaatgcactttcattgtaaaaattttggactatagagaatagtactaagaagaaaaaaaaaatcaccttcaattctgctgccacctggaggtaatcactgttaatattttgctatatactctatgagtttcttgttcaaaatcaggtcaaaattacatgcaattttgtaatctgacaatttccacttaatattttattagcattttcctgttatgaaacagtaattttagttatgggtcgttgttttgctatgcggttgggataaaattttatatactttttttggcaattacttattatacataaatgtttgtgtatagttttctttttctgagaattcctggaagttgagttaccaggcccggctttgaatttttttttttattttttttttgagacagagtcctgctctattgtccaggtgctatctcggctcactgcaacctctgtctccctggttcaagcgattctcctgcctcagcctcccgagtagctgggattacaggggcacaccaccacgcccaattaatttttgtatttttagtagagacagggtttcacgatattggccaggctggtctcgaacttctgaccccgtgatccacctgcattggcctcccaaagtgctgggattacaggcgtgagccatggcgcctggccaggctttaaatttaaaacaaatcttctaatagctttatggaggttataatttacatttcttgaaatgtactcactttgagtgtatagtaaactccaattttatcacatttctgtcaccccaaatgtatccttgtgcccatttgctgtaacctccggttcctgccccaactcctaggcagccactcatctattttctgtcccttaagatttgtgttttcgccaggcgctcatgcctgtaatcccagcactttgggaggccgaggttggtggatcacttgaggtcaggagttcgagaccagcctggccaacatggtgaaaccttgtctctactaaaaatacaaaaattagtcggatgtggtggcacacgcctgtaatcccagctactcgggaggctgaggcaggagaatcacttgaacctgggaggcggaggttgcagtgagcagagatcgcgccactgccttccaacctgggcaacagagagagactgtctcaaaacaaacaaagatttgtattttctggacattttatagtactggggtcatagtatagatggacttttgcatttggcttcttttacttaattgtgagattggttcttgttgtagcatgtatcagtagtttgttcatttttattggcgaaagtattctattatatgaataataccatattttatctatccatcagatggatattatagagttcatgttttggctaatttatgaattatggtactgtgaacatttgcctgcaagattttgtgtagacatgtcttcatttctcttgagtagatcacctagaagtggatttttaaataattttggtacttactgtgaaactgctcttcaaaaacataccattgttccttccttccttccttccttccttccttccttctttccttcctcccttcctccctcccttccctacttccctctccctttccctttcccttccccttttcccttccccttcccgcctgcctgcctgcctgccttccttccttccttccttcgtttctttctacatatacacatttttttaaatttcaatggtttttggggtacaagtggtttttggttacatggctgaattttggttacatggtgaagtctgagattttagtacacctgtcacccgagtagtgtaccttgtacccaatatgtagttttttgtccctcaccttccagccttccgccttgtgagtctccaatgtccattataccacactgtatgcccttgcgtacccacagctcagctcccacttctgagaacatatagcagaaacatgccaaagtatactcccactaccagaatgtgattgtgcctgattcttctcaccagtacaaatatttcaaaaaaagttaaatatgtatcagttttttgggcagaagttgatacttctctttatttatttattttttttgagatagggtctcattctatgatgcccaggctggagtgtggtggtgcgatctcggctcactgcagtctctgcctcccaggttcaagtgattcccacgtcagcctcccaggaagctggaattacaggcgagggccaccactgccagctaatttttgtattttttggtagagatggggtttcaccatgttggccagactggtctcaagctcctgacctcaagtgatccacctgccttggccttccaaagtgctgggattacaggcgtgagctaccacacccggctgatatttctttttaaaataacttaccttcttttgaaagtaatacatgtttaatgaacagaatttaaggaaaatataaaaaaacgaaataatctttgtaatcaaactactgaaaagaaaaccaaagttacattttggtgcatattctttttcattttcatcattgtaatttgcatttctttgattacttgtgagacactcctttcatttacttaataggtttatatgacttgcctattcagagattttgcagctttaccattttctgcaaatgatagcaacttctttttgtttgtttgtttgtggagacagagtctcgctctgtcactcaggcaggaatgcagtggtggaatcttggctcattgcaactattgcctcctgggttcaagcgattttcctgcctcagcctcccaagtagctgggattacaggagtgtgccaccatgcccggctaatttttgtatctttagtagagatggggttttgccatgttggccgggctgatcttgaactcctggcctcaagcggtccccctgtctcggcctcccaaagtgctgggattacaggcgtgagccaccgtacccagccagtagttacttcttatattctagaaaaaattctactcatgatcaagtctccatgaggaaagagactttaattgaagatcatggggcttgcagaccaatatgataaaatagttcattgtttctaaaagtattactgagtgttgatggcagatatgaacccttttgtttttgtaggaaaatgttacccgtattctccatttgaattcagtttagatttgttaggaatcgcagcttaagctttgccatctgggagtgtttgggacagttttgcagacaaaattgcaaaagtgcctaaggaatgcagctggcattcagacctgctctgtgctcagtactctgtggacagacactgttcagcacttgttgatcagaaggtttagaaagagaactttcaaagttggtttttaattaaagcatttaatagtgtaaatagaaagggattaaattttatgacagacaaaagaaagtacagcacccagctgggcgtgggggctcacgcctgtaatccagcactatggggggctgaggtgggtggatcacgaggtcaggagttcaagagttcaagaacagcctggccaaggtgatgaaaccctgtctctactaaaactacaaaaattagccgggcgcggtggcaggcgcctgtaatcccagctactcaggaggctgaggcaggagaatcacttgaacctggacggcagaggttgcagtgagccaagattgcaccattgtactccggcctgggccacagagtgacattctgtctcaaaaaaaaaaaaaaaagaaaaaaagaaagtacagcacccagttatgtccgagtgggtgcatgagagtgaccctgagattggagacaacgctgtcacgtgcttgaagaacgccacctgagaaagggggcgagaagtggtgtccgctggtaaccagaggtgttggcttagccatctgcagggaggagggtggtctatcacaggtgagtttcatctactttcttaagcaaattaaccttacttttgtgttaggcttgtcccaaagctgttttataaatgtgaccaaggacaagctgatccagtagtggccgtggcaagagatcaaagcagtgtttacctgaaacttctcatgcatgagacgcagcctccatctcatttctccgtcagcacaataaccaggtatgctgacccagtggcatcttcacattgtcgggaaaatgccctttcctgatgcctttctttaggctttaattgaaaacattttattttctagaaaaaagcttcagctcaggatgtttgagtgtaggtcagtcctttgataggatattatcattttgaggattgaccacaccacctctgtatttaagctctgccacaatcactcagctgtgacactgtaaatctcttaatagtttattacattccatgtgctgacagttgtatttttgtttgtgacacttacgtattatctgttaaaacattttcactttagttgtgttacctttaaagaggattgtattctatcatgcctgttgattttttggtgagcgggctattaaagtcagtgttatttagggttatccactagttcagtgatttgcgagattatcattcacatttattgtggagcttttgaatatcgtgtcaaatggccacatatatcccattcttatctgcttcttaggtgagtgggacacagtgctttaatgaagctataatcttcagaattctagcttgcagagaagattgcagaagtgataagacttgtgctttttaattttgtcttttaaatgttattttaaaaattggctttatatgatactctttttttctgctgagtaacagtgttttacaaaacttggactaaatgacttctaagcttaaatgatcacttgatgctttttttctgaattaggaactcagcttatcaaatatcaaagtcataattcctgaataaataacgtcttttttcatgtaaagactgctttaaaaaacacatggaaggctgggtgcggtggctcacgcctgtaatcctaacactttgggaggcccaggtgggcaggtcgcttgagctcaggggttcaagaccacccagggcaacatggcaaaacccacctctactcaaatacaaaaaattagccaggcgtggtggcgggcccctgtaatcccagctactcgggaggctgagggatgagaatcacttgagccccggaggcagaggttgcagtgagccaagattgtgccattgcactcccagcttgggctacagagtgagactctgtctcaaaaaaagacacacacacaaacaaaaaaaacatggagacatttttttggccaccttaatatttcccctcagataatttcctttgtttaaactcagaactggcattttctctcttggagaagattcaggacaaatactcctttaagataagtagaagcagtgaaagaggatttgattatcaggaatttgataagcttagaataaattgttgcttcttaatgtcatttcagaagatgaatatttattaatagatgccaactgagatatcattaaaattgattactaactactacttggaaaagtctcccagttccaaacttcagcaggcctcttgacaattcagctgtggtcaattgggtcttgcgtgatagatacaatgaccaattgtgcagcagagtgtgctgcttagctgcctattctgttagcattcatgtgttaacttaaaatcataatctccttagttttgttgagtgtctccgtggacaagacactgtgagggatacaaaatcagattggctttattcaaaccactggggtattataattcatttataatttattttattttttgccttttttccatgtgttctaaaggaattagagtttgtatataactataatgggggatagaaattgacatgtgccatgaagggaatgcaaaaaagtgccgtgggagatgagaagtggagaaaggaatttcttttttcttggaagcaggaataacttcatgaagcatgtatttcaacttaaacagatagtaggcaacgctgtaaggggagtatggctgcagcaaaagtgttcggggcagactgggaggaagggagggaataaattcagccattgttatggaataatgatcaaaatttattttcagcccgtttcacttaaaagttgagactgcttaactttttttaatctttaatcttaaacttttaaatgccatttgatctttaaaaatatatgttttaatagtgtattttaagtctctatatttttgttattagaatatatagaggctataacctactaccaagcataacagacgtcactatggaaaataacctttcaagagttattgcagcagtttctcatgaactaatcacatcaaccaccagagcactcacagtaagtctctttcttgatcggtcttactgacattgtaatagtttttggtagcttgtatggccagttagttgtatggtcatcttacggtgaggtgcttgtcttacagctcttacttatccatgaggcttgctaagaaattgtgcttctgtgaaaagaatctcagcttactccaggaatgtaaatgactatgttttttctgattattaaagtaatacacgcccaaaataaaaaaattcagccaatttaggaagacacaacaattaaaataagccaggcatggtggctcatgcctgtaatcccagcactttgggaggccaaggttgggggctcacttgaggtcaggagtcggataccagcctggccaacgtggtgaaaccccatctctactaaaaatacaaaaattagctgggcgtggtggcgggcgcctgtaatcccagctactcaggaggctgaggcaggagaatcgcttgaacctgggaggtagaggttgcagtgagctgaggtcaagccactgcactccagcctgtgcaatagagcgagactctgtctcaaaaaaaaaaaaaaaaaaagaaaagaaaaaagtaaactactgtcacctgcattggtaatgtatcagaagtttaaaatgtctagattataattaactcagtgacctggtaatatatactaagggaaaaatatttataatttacatttttacatttttatttttttaattttattattttttttttgagacagagttttgctcttgttgcccaggctggagtgcaatggcatgatctcagctcaccacaacctccacctcccgggttcaagcaattctcctgcctcagcctcctgagtagctgggattacaggcatgcaccaccatgcccggctaattttgtatttttagtagagacagggtttctccatgttggtcaggctggtctcaaactcccaacctcaggtgatccgccctcctcgaccccccaaagtgctgggattacaggtgtgagccaccatgcctggccttacatttttataataagaatttatgttgctgacattagaaaagaaccataatatccaagaatccaagaataattaaattatgtacatatgctagtatatagtgtgatgctttggagaatttttaacaatatggagatgtataatctggattgtaatattgagtgaaaaaaggcagaatacaaacctggtgggggtatagtcggatttcagttaagaaaaataatatttacatatatacatttctcacactggcagataatcaccaagataaattttgggattgtggatgatttttttcttctttatatttttcagatattctcaaattttctaaaatgagcaagtataacttttgttatcagaaaaaaataatatacaaaagtaatgttaatttgctggtgaccaggttaaacctttttatttttattttttgagatggaatctcactctgttgcccaggctagagcacagtggcatgatcttggctcactgcagcctccgcttcctgggttcaaatgattctctggccccagcctcctgagtggctggaattacaggcgtgtggcaccacacctggctaatttttgtatttttagtagaggtagggtttcaccaggttggtcaggctggtctcgaactcctgacctcgtgatccacccacctcggcctcccaaagtgctgggattacaggcgtgagctactgcgcccagccagacctttttattttatttgacaaaagaaatacttccatgttatagaagactaaatattgtttgggctgtctgcagtatggtcttcccttgatttgttcaaaatatcgtaaactttgcttatttatttttattgtggccgactgtgtcgggcactgttgtaggcttgggatggaaaaacaggattcctgcccttagggtttctgcaggctggtcagggagacgatgtggtaagctggagctcagctcctaaggatgtgcaggggcagttgagaggcggaagggtgggagatcattccagggtgtgggcagcacaggaacctctcttcattgggatataattgccattctgataacacgtgtttgaggtgtctaaagtaggaagttgtaccatggtgggacagatatcctgtggttatcatacacagatctcagttttcttctcattgtttgtactttttataaagggtaacaggagatataattcaataaacctttgtggtgtttgggtgtgattttattgtttctttcttctcagtttggatgctgtgaagctttgtgtcttctttccactgccttcccagtttgcatttggagtttaggttggcactgtgggtatgtattttcctcagtatatattaatagttgtctacaacagtatgacataaacatagttattaggatgccctttttctttctttttaagtcttttatcaatttggctttttggaaaaatatctgatggaatacttgtttctgctatattagctgtgtgagactagtgacaggagctgtgggaaatgaatgccaaatgttcttaggcattgatgggaatttcagggtgtggtcttcaagttcatttaagggaattttcatatgctggcaaaaggcttttctcattagcttgactctttccaaaattatttgctgtgaattagaagtttaggaaccttttttcacttaattgtgacctagcatacgaaatggtgatgatttaggaactactgttcttgtattaacagcttttatttaaaaatgattttcctccagtagatggccctactagcatctgggaaataatttcaagtcttctccagcattcaggaataggctttcattttgtgtatcaattactgagaatgattttggtgactcacatcacatttgagaagtaaacctgcagatttcttgtgtgtgtcagcaaatgaccaactgatatttgcttgaagtggattacattatctgctctagaatgattgctttcccaccttcctcacatacagactgagcagctacggtttctaatcataggtctggcactagacttcacttctgggcaactttggcattggagtaaaatgtattaatttaaagaaagttaaaaatccgttcaagtaaacatacagttctaatactttttacaatttaaaatatagatttaaatgataaaataaaaaagaaaatatgggtagacaccataatcctcgtttctgcatctgttcacaaggggttgatatttatgagttctattctccatatccattctatgttctcttaatgctcagtcagcacctcaggtggttggagttcaatgcttggtagtttgacttacactgtcttttctaggggattgagccctgggtagtcctgcttatttgaggttgcaatttgtctttcaataacttttactacaagatatggcgtgttaaaggataccattggggaaccaacataataatatcaggaaaactaaccacgtcagacctgccccattgtgtatcaagtacactatttttccatagtaataaagagttcaccccagccaattctcttttattttgtgcctgtttactcaatggcattaacatgcccaaatgtctgggtagctgtctcatctccagttcagcagaaccattgtcatatgccctagtaaaagcattccttcattggacacttaggccccaatactttcattcagatctactacctgatttcatttctcaaatgatttttatggagctctgatttataggaaagatgttagttgattaaaaataaaacaatttctgagctggtataaaatgtattgtgacatgccttcctcttggaattgcaagagaaaggaagactgttgtttgcttaaaaattgtctataatttgactttgcaaatgtctgcttccagagtgcctccactgagtgcctcagatgagtctaggaagagctgtaccgttgggatggccacaatgattctgaccctgctctcgtcagcttggttcccattggatctctcagcccatcaagatgctttgattttggccggaaacttgcttgcaggtactggtactgagttgaaacagggactccaggacttggattttgatttccttagggggaatgggggtggtgagcatatgaggggaaaatactataaggtcattgccagtgatggcttgtccctttagtcaaatttcagatgttacctatatgcataaacacatgcagttggcagctgttctgtgctgagtattttaaagtagcctcttcccaatatagcccctcagttaactacaagtaaactcattttgaatttcattttaatgggcaccatatgccagtactccctcgggcactgggatgttaagaaagtataatgtatggacttcattctcaagttagttttagattagagggggatacacgtaaacaaaagtgcagtggtcacacagagtggccctaatcactctccttgggcagatttatgggctggtaggaaagagcacaacacggagagggtgtagcaccttggcgatgataatggaggatgtggccagcaaggaagacggagtccattgaaattgattttgggagaagttgccaatctccatgaaagaattggggcctgtgctatttgcttcagggggctataggagagtttcgtgaaagggactaaaagatgagtattttaataagatcattcatccaacttgaacatgggctggaggagaaggtagggagactcaggagattaatgttgatgctaaggcaagataatggctttgggactgtagggaagacactgattgtaagagaatgaaggaggcagaattgccaggcctggttcaccaactgaacttcggttgtgaagacaaagaaacctgggatgacttcacatcctgggcaggtgtgtggtggtgacagtcatggaaattgggaacacagatttgtgcgggaaacatcagtttcagtttgagtttggcttatcagttgaatatcaggcacagatgtctggccaactctcaacatagggtcttaaatgacttcagttccccaagcaatttgtccttcccatgctattggggtggagaggtaatgtctgtgcccatatcacagccagtgctcccaaatctctgagaagttcatgggcctctgaagaagaagccaacccagcagccaccaagcaagaggaggtctggccagccctgggggaccgggccctggtgcccatggtggagcagctcttctctcacctgctgaaggtgattaacatttgtgcccacgtcctggatgacgtggctcctggacccgcaataaaggtaatgtcccacttgggtgctggattcatacagccttaatgactatgggtttccagactacctttgtttagtaatctgtcccttctttattctctttttgctttaaatgaacaaaattgctcagattgtgacactaaatttaacatcaaaatgtgaccatgtggatgggtgcagtggctcgtgcctgttattccagcactttgggagactgaggcaagtggatcacttgaggccaagagttcgagaccagcctgggcaacatcacgaaaccccctctctactaaaaatacaaaaaattagatgggttgggccgggcgtggtggctcaagcctgtaatcccagcactttgggaggccgaggtgggcggatcacgaggtcaagagatcaagaccatcctggctaacacagtgaaaccccgtctctactaaaaatacaaaaaaattatctgagcatggtggcgggcgcctgtagtcccagctgctcgggaggctgaggcaggagaatggcgtgaatccgggaggcggagcttgcagtgagccgagatcgtgccactgcactccagcctgggtgacagagcgagactccgtctcaaaaaaaaaattagatgggcatggtggtgcgtgcctgtaatcccagctacttgggaggctgaggcaagagagttgcttgaacctgggaggcggagtttgcagtaagccttgattgtgccgctgcactccagcctgggtgacagagtcagactctttccaaaagaagaaaaaaatgtgaccatgtgttttatagctcttttagtatcatcagtcactgttatccctaagagggaaatacctagctttagttttaggtttccagcattagccaagaaagctcagaattgatgttcctggccaagtacctcattgctgtctccttaaatcttggttaatggctactgtcctggctagcatagttatggagcatttccatggttgtagaatgttctgccaatctcagggacagttttgcttttctgtgaagcaataaaatcaacttcaaaacaaatgttaactatttgtacaatggatttaagatagaccagttcacatacttttttttttttttttttttgagatggagtttcattcttgttgcctgggctggagtgcaatggtgtgatctcagctcactgcaacttctgcctcctgggttcaaacgattcttctgcctcagcctctcgaggcagattacagctgggattacaggcatgcaccaccacacccagctaatttttttgtagttttagtagagacggggtttcaccatgttggtcaggttggtctcaaactcctgacctgaagtgatctatccgcttcggcctcccaaagtgttgggattacgggcatgagccaccacgcccagcctaagatagaccagttcacttactgtttatatctgattactctctctttgccttgtcttctacctttaaaaatctccctactaacttcccattctcctttagctgccatcagtcttctcccttctctgcaaacatctctggagagtcccagcctcagcccacagagcttcccactgctctgaggtggaccttgtttgcaaggcttctttggctctcttggcctggaccctgtctactacttcagccatccttccttaacccctgctggtggtttctgttgccacactccatagcagcgtttcccgcccagatcatgtctttacatctctgggcactgctctggtcctgcctgcctttccctctttgtatcctgcaggctgctacccccatcttgagtgtcctcttcagttggctttcagagggcctcctgggtgttcccttacccacttgccactccccagtcactgggttcagtccttcctgcccaccagcacatgctttctaggctctgtcctaggccgtcttctctctttgtagtctctgggccagtgctgttctagagagtggcagaattttctataaccatggcagtgctccatagctatgccaggcaagacagtagccactaaacacatatagctgttgagcccttgaaatgcagctagtgtgactgaagaactgaaccccgattcggtttaattttcattaaatttaaatttaaataaccttatgtgggtagtggctccagtattgggcagggcagcctgagagtcggggctgttctcctgtcttcagtgtctagatgagggacctcagaggacctgtctctggagctgcagttcaatgtagccagctgccccgtgacacttacatatagctgatttgtggatatgtcagacacggtgtgatgagctcagctttctgtcctcctccccacatctgcccctgccccatttaccccactttgtgtcttatcaagctagaaacaggtcaccacaagtcttcatttccactcaccaagtcttttgtttcccctactaaatattttgcgagaagaaagtgtgtacctttgtattcacatacatgtacatgcacatatacatgcacatatgcaggggtccccaacctctgttaaaaaccggactgcaggccgtgcgtggtggctcacgcctgtaattccagaactttgggaggccgagaccagtgcatcacaaggtcaggagatcgagaccattccggctcacacggtgaaaccccgtctctactaaaaatacaaaaaaaaattagccgggtgtggtggcgggcgcccatagtcccagctacctgggaggctgatgcaggagaacggcgtgaacctgggaggcggagcttgcagtgagccgagattgtgccattgcactccagcctgggcgacagagcgagactctgtctcaaaaacaaaacaaaacaaaaaaaaaaaaaaccaggctgcacaggaagaagtgagcaagcattaccatctgagctctatctcctctcaggccagtggtggcattagattctcataggagcgtgtatgagttcgttctcacacttctgtaaagacatacctgagacatataaagaaaagaggtttaattggctcacagttctgcaggctgtacaggcttctgtttctgggaaggcctcaggaaacttgcagtcatggcagaaggtgaaggggaagtaggcacatcttcacatggcccacaggaaaaagagagaaggagagagagagagagacagagagagagagagaaaaagaaagattgagagggagagaggagggagaaaggagagtgcctgtagggggagttgctacacaaaggagcaccagggggatggtgctcaaccattagaaactacccccatgatccaatcacctcccaccaggccccacctccgacactggagattacaattcagcatgagatttgggtggggacacagagccaaaccatatcagagcatgaaccctattgtgaactgcacatttgagggatctaggttgcatgctccttatgagaatctaatgcctgatgatgatttgaggtggaacagtttcatcccgaaaccatcccccgccaaccctggtttgtggaaaaattgtcttccacagaaccggtccctggtgccaaaaagtttggggacctctgcacatatgcatgcacctgtacatggacacataatacatgtacatatgcatactttatattctctgccacttctggtccagactgatatactatctcatttggattactgcactagccttttgttttggaaacagcattttttaaaaaatttaatttaatttttttgagatagggtgtcattctgttgcccagcttggagtgcagtgtcatgatcatagctcactgcggcctcgatctcccaggctcaagtgatccttctgcctcagccttctcagtagttgggactacaggcatacccaccatgcccagctaattttttgatttttttttttttttgagacagagtctcagcctgtcgcccaggctggagtgggttggcgcgatctcagctcactgcaacttctgcctcccaggttcaagtgattctcctgcctcagcctcccgagtagttgggattacaggcgcctgccaccacacccagctaactttttgtatttttagtagagacggggtttcaccatgttggccaggctggtctcgaacttgtgacctcgtgattagcccgcctcggcctcccaaagtgctgggattacaggcgtgagctaccgctcccagccaggaaacagcattcttgagataattcatataattcacccatttaaagtatataattcattctctttagtatgcccacagagttgtacagccatcaccagaatcagttttagaacccataaaggaactctgtactctttacccaaaacctccatgcctccagctgcaggcagccactaacctgccttctgtctctgtgactctacgtcttctggacattactgtggatgggctcatacagtcagtgagcttgtgactggtgccttctaccaagcagggttttcagtgtagcagcctctctgtttttcttttttttttaaattgtgacggaacttctgcctcccgggttcaagcgattctcctgcctcagcctcccgagtggctgggactacaggcccatgtcaccatgcctggctaattttttttttttttttttttagtagagatgggtttcaacatgttagccagggtggtctcgatctcctgacttcatgatccgcctgcctcggcctcccaaagtgctgggattacaggcgtgagccaccatgcccggctaacctttcatttactgtctgcatttcttccctgatgccttccagtccatgcacccgattgtagccattcatcctattatggtttaaggtgactgtcttagtcagcatgggttgccataacaaaataccatagcctgggtggcttcaacaacagaatttacttctcacacttctggaggttgggaagtccaagatccaggactttcgccttgccctcatgtggtgagggggtgaggaagctctgtggggcctcttatatatggatgctaatctcattcatgaggggtctgccctcatgacccagtcacctcccaaaggccccacctcctaataccatcaccctggtaattaagtttcagtgtataaatttgggggactatagacattgaaaccataacaagcacttttctaagatcagggagtgagtaagtagcagagctaggacctcaattccacatgtcagtcatcttgccttcactctgctccatgatggctgcctcctagagcattgggagtctcgatgttctatatgctctcatgtgttgtgtattggagatagttgaggctttatgaatacatctggatttgttgacttctagctttgctggtaaccagctgtgaccttgaataagttacttcatctctgagcctgtttcctcttttagaaacaggagtttaaaatgctgctttgggttgggcacggtggctcatgcctgtaattccagcactttgggaggctgagatgggaggatcactggagcttggagttcgagaccagcctgggcatcatagtgtgagatcctgtctcctcaagaaattaaaaaattagctgggtgatgtggcgtgtgcctgtggtcccatctactctggaggctgaggtgggaggattgcttgagcccaggaggttgaggctacaatgaaatatgattgcaccccatcctgggtgacgagtgagaccctgtctcaaaaaagaaaaaaaaaatgctgctttgtacccctttcatgtcatggcgtcatggccaacatagaatgccctggttgtttgctgttggagggcatgggcctgggggctccctgagggctccttccatcttcaactcattctctgtgcacctgttaggaagttgtgggccagtccctaccatgtatcattgtgtgggtaaaagtaaataaaatgtgtacagtgtctgaactgtacatatcagggtccaagaacaaaatgagtgacatgggttagctctttttaataaatggtaaaaccaaatattctaattttcagttttgttatacttccatcacatgtttttgtttttttgttttttgtttttgtttttctattttaggcagccttgccttctctaacaaaccccccttctctaagtcccatccgacgaaaggggaaggagaaagaaccaggagaacaagcatctgtaccgttgagtcccaagaaaggcagtgaggccagtgcaggtaggaaacagcgtggggaagggagggacatgagtgcagcatctgtcatgtagaaacataggatttaagtaacttggtgttttagagaaataaatataatacacatcagtaaagtgagagaaagtttctccaggtgcggttcaagatattagaaactaatgactgatgtacacagaccaccttttggtctgaagcatttctaagtgccactggctgacatgcagcccctacagcctccaggcttccagccctagcatggagcatcactctcctatgcttccctggttgcaggtgatggctggagaggcctcctgattttcagtaagggaagtggtgtagatgcttaggaatagatgtagtgagtgaaaaaactgattctgatatgtcaaaaattctgattggaaatggaatatttacatttggaagagctaaaggcgagagaaagtggggataaagtcatctgagttggaggagcttaaaccattcacaagtttggaggacctttttttacccatgaaaaggtcagaacagaaggggctaggatttaggtgtgactgcagtttattgaattcccatccatactgctctcggtgggcagtggcaggggcaggagaggagcctggcaaagcatgaagtgactgctgctgcctctgctatctgggacgcctggccacctgtctgtacagtctccctccagacccattctcacgctgtctcttggcacccaggggccagtgatggttctcccatttgttttgtgtatatagcatttatatcaaggctatttatttatttatttattttatttatttatttttttgagacagagtctcactctgtcacccaggctggagtgcagtggtgcaatctcggctcagtgcaagctctgcctcctgggttcaagcaattctcctgcctcagcctcctgagtagctgggactacaggtgtgcaccaccacacctggctaattttttgtattttttattagtggagacggggtttcaccttgttggccaggatggtcttgatctcctgacctcgtgatccgtccacctcagcctctcaaagtgctgggattacaggcatgagtcactgtacccggcctatttatttatttttaattgacaaaattgtatatatctgtaatatacaacatgatgtttgaaatatgtgtacattggccaggcgtggtggctcacacctgtaatcccagcactttgggaggctgaggtgggcggatcacgaggtcgggagttcaagaccaaactggccagcatggtgaaatcctgtctctactaaaaataccacaaaaaaaaaaaaaaaaaaaaaaagccgggcatggtggctcgcgccagtcgtcccagctacttgggaggctgaggcaggagaattgcttgaatctggcaggtggaggttgcagtgagctgagttcatgccactgcactctagcctgggcgatagagcgagactccgtctcaaaaaaaaaaaaaaaagaagaaatacatatgcattgtggaatggctaattaacctgtgcatcacctcacgtatcattgttttgtggtgagaacacttaaaatctactctttcagtgattttcttgcatatggtacattgctattaactgcagtcaccatgctatacagtagatctcttgaactcattcctcctgtctataaatgaaattttgtatccttgaccaacacattcaaggttttttttgagatggagtcttcttcacccaggctggagtaccatggcacgatctcatctcactgcaacctccgcctcccaggttcaagcaattctcctgcctcagcctcctgagtagctgggattacaggcacatgctactgcacctggctaatttttgtatttttagtagaagtggagtttcaccatgttggccaggctggtctcgaactcctgacctcaagtgatccgcctgccttggcctgccaaagtgctgggattacaggtgtgagccactgcacccggcctcaagcgttttaaaagatgctcttttctaaggattgactgtagtacaggaggaagattgacctgttgaaaagcctcagcctttacaagtgtaaaattatcagtatattactatcatctttctgatgaattaaataaactaaggactccaagtcaaaagtcttcaaactgaagtagaatagttgtatatagtgcttggcactttaatatttagtatcggtttaatgataatgtttgtgcctttgccgtctttaaaacatttttacatcatccctgtttgattacttggtgtgctcatgaagttgttggccactaaggaatcttaggctcagagaggttctggaattggccagtggtccttgaatcagctgctcctatgattctctaactgatttctcacaaagcaaacaagcaatcataacaaaacaactgtgcacactgctcttcttattttgttatttaaaaagtacttaggctctacttatgtttgttagtcaatttctcattacttctagttaatcaaaaggtcagaggaaatacttgaatattttcatactagaatactttaaaaaatcatgatttccagtaatctctttaaaacttggcaagttattttgatctaaaagtttatcttttgtgtgcatatttttaaagcttctagacaatctgatacctcaggtcctgttacaacaagtaaatcctcatcactggggagtttctatcatcttccttcatacctcaaactgcatgatgtcctgaaagctacacacgctaactacaaggtatgggcctctgcatcttttaaaaatatatatgcacacatacttacgtctaatggatagttgatgtttttcttatgatttgtaggatgtataagccctttgagatatgagttacatttagttttttcaagtttgtttgtctttcagctttgtttatgatagcttctatcatacaggtgttttggattttcatattgtttgtactcacagctaagattgattacagtgacagagctaggatgtgcagccaggttatagggggaagtggccctggtggagtctggagggatccgtgtacaggcttccttccctcccgtgaggctcacacaaaaatacagcaacatgctggtcctgcaggtaccctctgcctaacatgagccacaattccagactcacagaagaaaagcaggtgttcggcataaaccatgtgtttcaaatagtctgggcatggtgagccacttgttatcagctagggaaagtttatgtcagcgtaagaaactgttcaccagatacccccaagagccagcctttctgtctagggatgttttagttttttagttcatttttttttttaactttaaaattttctgttcatctgcaatttgttagatatgaagtatgtgtctaatttaatttttgtttttggttgtccccaataatgtttacagaagaatttttctgcactaattggcttgagttacttacattctcatagttctctagtttcagtagtttcatttattattttgttatatcaatctatctgtctgctcatctattagaagcatccttgttttttttttttcttttttagacagagtcttgctctgtccccaggttggagtgcagtggtgcaaccatgcctccctgcagtctcagggctcaagtgatcctcccacctcagctcctgagtacctgggactaccggcatgtgccaccacacccagctaatttttacattttttgtagagacagggtctccctaagttgcctgggctggtctcaagctcctggcttaagtaatcctccctccttggcctcccaaagtgctgggattacaggtgtgagcaactgcacccggctacaagtatacttcttaattattgtagcttaatggtatttatgaggggatcagttcccctgttgttctttagaattttctggatattcttctttattgattttgggatgtgaacaatagaatcaacttctacttgtagattgatttagggagaacttatacctcagatgttaagtcaccctgtccagaatgtgggatgctttcctatttgttcagaactttttaaattacctcagaagcacatgaaatttaaaggattttaaaaaaaacttaaagattatttcacatagctcttgcacatttcttgataaatgaatcctcaggtattcctctgtttttgttactaatagttacttcttatgggttttttttcccctgaaaatcatttatcaaacgtatgtggcttattttctgaaggatgtttgataattttggaagatatgaaagtcttcatattttacaaggtttgaggtctctttaagctgcatggttctcatgtcagctcccaaagcagaagacggcatgttgaaaaatgccgtagagaagatacttcttttccacctgttttcaactcatatcatcttgaatttcagggcacctttccatgctcctagtgcttgctatctgtttattattttccttcctgaataccctgaactccagcatgttctgctgtaattctggcctccctggcatcttggactcctgtttcctttgctctgtcatccccgcggtcagctcctgctgcgcagcttctcagctgaagtgcgtttggagtgcctggcgtgtcttgctggatctttgagtattgcctctggtttccttggttccttctgctgagttgctcagcgtctccactccccatttcttgtgtggcccttcctgcactcctctgattccttttgtcttccctggtttcttgctttggtttcgagtctccacagaacttttgcagctcttctgaagacctggaagctttttcatcttaattctcatctcatgacctcttttcccttctttgagagctagaacttcccatggtgaacttctctttccagaattccatgccttcttttccctcccacttacctgttgtccaggagaggtcagattgctgtgcatattggaggagaaccctttcttccctgggctcttcatctcacatgacatcaccacatcacctcgttccttggaccctcagtggtgtcactgctggatttttctttcctttggctggccttagggcacacccaggttgactagcgtagtcatggtatttagatccactcacattttcagtttctgtgtctgtctcttgcctgcttctgacttcgcccagagaaagcttctctttcacaagggttcttagatttatgttcactgagcaccttcttttctgaggcagtgttttaccaatatttattttcctagtcagtctcgccttacctttcttgttatgcatgtctttggtcctgacccattctctgagtctgtaaaatagaattgctgtataatttaattacatgaaatcctttagaatcttaacacatcttacacctgatttaatattttattgtatccaaattgaaccaaccctatgtgaatttgacagtgatttctcccagggatcctagtgtataaggaataggacttagtattttctattttttgatataccacataccagatactgattatgatggacatttaacccttttttctcattatgaaagaaagttaggaattatttcttccagtagcgccagtgtaacctgaaagcctttgaaagagtagtttttgtatagctatctgaaaggaatttctttccaaaatatttttccagtgctgacaacaaacacgcagacacaccctgcaaggtgagtgtacggcgccgcacagtggaggcatctgctgcagccgtcgatgtttgtgtctttggttgtacattatgagatcgtgacagggccagtaaccgtgtgttctctccttcaccttcccaaggtcacgctggatcttcagaacagcacggaaaagtttggagggtttctccgctcagccttggatgttctttctcagatactagagctggccacactgcaggacattgggaaggtttgtgtcttgttttttctccttgggttgtggctggcacacttgatgtgcgtcttctgggctgagttcatctaggatggagcctggttctccagggtgcctccgggagactcctccctgccccacgtgcttgcgtcacaggacccaagtctgactctgccttagccatgaagtttagggggaagtttctatttgtattctatttttgtctgttatcatgtattagcttagacccagtttagtttggaaaatcagtgggtttcaaaatgtgtttgtagagtcctttatttcttaacttgaccttttcaagtggaaaggggcaaaacagacgggtaagggggcggggcgggaggtgtgacttgctcttttgtgcctgaggaagtaacagagctggggttgacagtcatattctctgacacagatagtctctgacttatctcacagaaagtcagcggcagagcctgagttaaaagtctcgtagattttctttttcttttttttggtggctaatttcagttttatttatatttgtttatttatttattatactttaagttctgggttacatgtgcagaatgtgcagttttgttacataggtatacacgtgccatgatggtttgctgcacccatcaacccatcacctacattaggtatttctcctaatgttatccctcccccagtcccctcactccccatgggccccggtgtgtgatgttctcctccctgtgcccatgtgttctcattgttcaatttccacttgtgagtgagaacatgcggtgtttggttttctgatcttgtgatagtttgctgagaatgatggtttccagcatcatccatgtgcctgcaaaggacatgaactcatccttttttatggctgtatagtattccatggtgtatatgtgccacattttcttaatccagtctatcattgatggacattcgggttggttccaagtctttgctattgtgactagtgccacaataaacatacatgtgcatgtgtctttatcgtagaatgatttataatcctttgggtatatgcccagtaatgggattgctgggtcaaatggtatttctagttctagacctttgaggaatcgccagactgtcttccacaatagttgaactaatttacactcccaccaacagtgtaaaagtgttcctatttttccacaacctctccagcatctgttgtttcgtgactttttaacgatcgccatcctaactggcgtgagatggtatctcattgtgattttgatctgcatttctctaatgaccagtggtgatgagcattttttcgtatgtctgttggctgcataaatgtcttcttttgcgaagtgtctgttcatatcctttgtccattttttgatggggttgtttgcttttttttcgtaaatttgtttaagttctttgtagattctggatgttaatcttttgtcagatgggtagattgcaaaaattttatcccattctgtaggttgcctgttcactctgatgatagtttcttttgctatgcagaagctctttagtttaattagatcccgtttgtcaattttggcttttgttgccattgcttttggtgttttagacatgaagtctttgcctatgcctatgtcctgaatgttatggcccaggttttcttctaggatttttatggtcctaggtcttatgtttaagtctttgatccatcttgagttgatttttgtgtaaggtataaggaaggggtccagtttcagttttctgcatgtggctagccagttttcccaacaccatttattaaatagggaatcttttccccattgcttatgtgtgtcaggtttgtcaaagatcagatgattgtagatgtgtggtggtatttctgaggcctctgttctgttccattggtctatatatctgttttggtaccagtaccatgcagttttggttactgtagtgttgtagtatagtttgaagtcaggtagtgtgatgcctccagctttgttcttctagcccaggattgtcttggctatgcaggctcttttttggttccatatgaagtttaaaatagttttttccaattctgtgaagaaagtcagtgatagcttgatggggggatagcattgaatctataaattactttgggcagcaaggccattttcacgatattgattcgtcctatccatgaacatggaatgtttttctatttgtttgtgtcctctcttatttccttgagcagtggtttgtagttctccttgaagaggtccttcacatcccttgtaagttgtcttcctaggtgtttcattcccttagtagcatttgtgaatgggagttcactcatgatttggctctctgtttgtctgttattggtgtataggaatgcttgtgatttttgcacattgattttgtatcctgagactttgctgaagttgctaatcagcttaaggagattttgagctgaaccaatagggttttctaaatatacaatcatgtcatctgcaaacagggacagttttacttcctctcttcctatttgaataccctttattgctttctcttgcctgattgcgctggccagaacttccaatactatgttgaataggagtggtgagagagggcatccttgtcttgtgccggttttcgaagggaatgcttccagtttttgcccattcagtatgatattagctgtgggtttgtcataaatagctcttactatgttgagatacgttccatcgatacctagtttattgagagtttttagcatgaaaggctgttgaattttgtcaaaggccttttctgcatctgttgagataatcatatggtttttgttgttggttctgtttatgtgatggattacgtttattgatttgcgtatgttgaaccagccttgcattccagggatgaagctgacttgattgtggtggataagctttttgatgtgctgctggattcagtttgccagtattttattgaggattttcacatcgatgttcatcagggatattggcctaaaattctctttttttgttgtgtctctgccaggctttggtatcaggatgatgctggcctcataaaatgagttagggaggattctctctttttctattgattggaatagtttcagaaggaatggtaccatctcctctttgtacctctggtagaattcggctgtgaatccatcctggactttttttggttagtaggctattaactattgcctcaagtttagaacctgttatcagtctattcagagattcagcttttttctggtttagtcttgggagggtgtatgtgtccaggaatttatccatttcttctagattttctagtttatttgggtagagatgtttatagtattctctgatggtagtttgtatttctgtgggatcggtggtgatatcccctttatcgtttttattgagtctatttgattcttctctcttttcttctttattagtcttgctagcggtctacctattttattgatcttttcaaaaaaccagcacctggattcattgattttttttggagggttttttttcgtgtctctatctccttcagttctgctctgatcttagttattttttgtcttctgctagcttttgaatttgtttgctcttgcttttctagttcttttaattgtgatgttagggtgttaattttagatcttttctgctttctcttgtgggcatttagtgctataaatttccctctacacactgctttaaatgtgtcccagagattctggtatgttgtgtcttcgttctcattggtttccaagaaaatttttatttctgccttcatttcgttatttacccagtagtcattcaagagcaggttgttcagtttccatgtagttgtgtggttttgagtgagattctcaatcctgagttctaatttgattgcactgtggtctgacagacagtttgttgtgatttctgttcttttacatttgctgaggagtgttttacttccaactatgtggtcagttttagaataagtgcaatgtggtgctgagaagaatgtatgttctgttgatttggggtgcagagttctgtagatgtctattaggtccgcttggtccagtgctgagttcaagtcctggatatccttgttaattttctggctcattgatctgcctaatattgacagtggggtgttaaagtctcccactattaccgggtgggagtctctttgtaggtctctaagaacttgcttcatgaatctgggtgctcctgtattgggggcgtgtatatttaggatagttagctcttcttgttgaattgatccctttaccattatgtaatggccttctttgtctcctttgaactttgttgatttaaagtctgttttatcagagactaggattgcaatccctgctttttttttgctttccatttgcttgttagatcttcctccatccctttattttgagccaatgagtgtctttgcatgtgagatgggtctcctgaatacagcacaccaatgggtcttgactctttatccaatttgccagtctgtgtcttttaattggggcatttagcccatttacatttaaggttaatattgctatgtgtgaatttgatcctgtcattatgatcctagttggttattttgcccgttaactgatgcagtttcttcatagcgtcagtagtctttacaatttggcatgtttttgcagtggctggtactggttgttcctttccatgtttagtgcttccttcaggagctcttgtaaggcaggcctggtggtgacaaaatctctgcatttgcttgtctgtaaaggattttatttctcgttcacttatgaagcttagtttggctggatatgaaattctgggttgaaaatactttttttaaagaatgttgaatattggctcccactcttttctggcttgtaggatttctgcagagagatctgctgttagtctgatgggcttccctttgtgggtaacccgacctttctctctggctgccctttccttcatttcaatcttggtggatctgatgattatgtgtcttggggttgctcttctcgaggagtatctttgtggtgttctctgtatttcctgaatttgaatgttggtctgccttgctaggttggggaagttctcctggataatatcctgaagagtgttttctaacttggttctattctccccatcactttcaggtacaccaatcaaacgtagatttggtcttttcacatagtcccatatttcttggaggcttggttcatttcttttcactcttttttctctaatcttgtcttctcgctttatttcattaatttgatcttcaatcactgatatcctttcttctgcttgattgaatcggctgtcgaagcttgtgtatacttcacaaaattctcgttctgtggtttttagctccatcaggtcatttaagctcttctctacactggttattctagccattagtctaacatttttttcaaggtttttagcttccttgtgatgggttagaacatgctcctttagctcggagaagtttgttattaccgaccttctgaagcctacttctgtcaattcatcaaactcattctccatccagttttgttcccttgctggtgaggagttgtgatcctttggaggagaagaggtgttctggtttttggaattttcagcctttctgctatggtttctccccatcattgtggttttatctacctttggtctttgatgttggtgacctacggatggggttttggtgtgggtgtcctttttgttgatgttgatgctattcctttctgtttgttagttttccttctaacagacaggcccctcagctgcaggtctgttggagtttgctggaggtccactccaggccctgtttgcctgggcatcaccagcagaggctgcagaacagcaaatattgctgcctgatccttcctctggaaacatcgtcccagagcacgaaggtgtctgcctgtatgaggtgtttgttggcccctactgggaggtgtctcccagtcaggctacatgggggtcagggacccacttgaggcagtctgttcattatcggagcttgaatgccgtaccgggagaaccactgctctcttcagagctgtcaggcacgtatgtttaaatctggagaagctgtctgctgccttttgttcagatgtgcccttcccccagaggtggaatctagagaggcagtaggccttgctgagctgcagtgggctctgcccagttcgagcttccctgctgctttgtttacactgtgagcatagaaccacctactctagcctcagcagtggtggacacccctcccccagccaagctcctgcatcccaggtcgatttcagagtgctgcgctagcagtgagcaaggccccatgggcgtgggacccgctgagccaggcacaggagagaatctcctggtctgctggttgtgaagactgtgggaaaagtgcagtatttgggcaggagtgtactgctccttcaggtacagtcactcatggcttcctttggcttggaaagggaagtcccccgaccccttgtgcttcccaggtgaggcaacaccccgccctgcttcggcttgccctccgtgggctgcacccactgtccagcaagtcccagtgagatgaactaggtacctcagttggaaatgcagaaatcacctgtcttctgtgtcgatctcactgggagctgtagactggagctgttcctattcggccattttggaagcatcccttgttttttgaggtggagtcttgctctgtcgcccaggctgacgtgcatcggcacaatctcggcccactgcaacctttgcctcctggtttcaagcgattctcctacctcagcctccggagtagctgggattacaggcacctgccaccatgcctggctaattttttgtatttttagtggagatggggtttcaccacattggccaggctagtctcgaactcctgaccttgtgatccacccacctcagcctcctagagtgctgggatcacaggtgtcagccaccacgcccagccatattttcagatctccctctctttgccctaaaccactgtgcttaataagtagtttttagtggccagcagtctccatgtataacacattttagcaaaatggaaaatactatatgttttaaatttgaacgtgagattatactgaaataaaaatcatctaactgggattctttaaatagtaagattttcttttttgtatgtgggtttttttttaaccttattattatgactgtcatatatagaaatggctgtttttcagttacagtcagtgaatgtatcaaatgctgccttatccaaataataaaagtaaattattaataagtcacaatttaatgaagattgatgttagttgatctttatattcttgaaatcagccatatggttgtgtgtgtatgtatatatttttaaaggtacataaagataataagctcatctctgaaaatttttacatttggcataagaataactggataattaagcatcttattctctggcctgtgtctttacagttaaaggtagatttactcacctctccttttttgtttttctaagttcatcttttttgctgtttcaagacagaggcccattttagctttctcgcatatccttttgtttgtactttggaagcctcacctgcttaattgttgagtttttatccgtggtcttttagagggggatatgtagggtagaagctttcacaggttcttgtttgcacttggcccctgactgttttgaggaatctccctcactgactcacagcatggcaaggtttcagatctctttctgccacacagcagttctgaggcagctggaaagatatccagatgcttagattgtcaggccaggcttgagatatacaaactattgagccttatctgtgaccttgcttaggtgaaggcatcagagcccctgcaccaacatgcataggcctctgcatgtgtgcggggctgggtgttgaggtctgagcacaagtgtagctggagaggtgagcttgatgtggcgacgggtatgagcaggttttcttcagacttctgtgagtttacctagttccaggatttaaaggcacagagactttagaattaaaatagaatcattttctttttctaaatagcaacactaggaataaaaaataataattccacattcttgacaggtaatgttttttcttgtcttctaatccttatttattccatactcatttttatacataattgaaatgtattatgcattggatttttcttttgcattatattatagacgatttttcatgtaactccttactgttccattttatatgttttgtctggtttaagactttatctgcaaaccgggaaactgtctctacaaaaagaaaaacaaaaatagttggccgcagtggcatgcgtctgtggtcccagctactcggggctgaggtgggaggattgcttgagccttgggaggttgaggctgcaaagagccatgatcatgccattgcactccagcatgggtgacagactttatactgtctgttttgggtgatttgataatgatatgccctgatgtagtttttttatatcttgtgtttcttgtgcctgggtttattgaggttgggtctgtggcttcatagtatttttaaagtttggaaaattttaggccattctttctttctttctttcttttttttttttttgagacagtgtctcgctctgtcgcctgcgttggagtgcagtgacactatcttggctcactgcaagctctgcctcctgggttcacgccattctcctgcctcagcctcctgagtagctgggactacaggcgcctgccaccacgcctggctaattttttgtatttttagtagagacgaggtttcactgtgttagccaggatggtctcaatctcctgacctcgtgatctgcccgcctgggcctcccaaagtgctgggattacaggcgtgagccactgcacccagctaggccattatttcttcaaagattttttttctgccctgcctccctccttttttccctctcttaaaggggctgtgatttcctgaatgattgcttagtgttgtcccatagcttactgatgctcttttcagtgtttgattgttttatgtgttttctgttttgtatagtttctattattgtgttttcaagttctctgatcttttcttctacagtgtctactctgttgttaatctgttaatctgttgttaatcctgtccagcgtatttttttttttgtttttgaaacagtctcactctgttgcccaggctggagtttagtggtgcgatatcagctcactgcaacctccacctcccaggctcaagcaattcttctgcctcagcctcccgagtagctgggactataggcacgtgccaccacacctggctaatttgtgtatttttattagagatggggtttcaccatgttggccaaactggccttgaactcctgacctcaggtgattcatccgcctcggtctcccaaagtgttgggattataggcatgagccaccgtgtctggcccctgttcagtgtatatcactaattttgtttttatctctagaagtttgatttaggtcttttaaaaatgtctccctgtgtttctgtttagctttgtgaacacaattgtaataactgttttaatatccttctctgctagttctaagatcttctaataacttcccagttcttggtgtttctcattggttgattgatactcctcgttttgggttgtattttcctgcctctttgtatggctgccaattttttattggatgcccaaccttgtgaattttactttgttggatgctatatatttttgtgttcccatagatcttcttgagctttgttctgaggttagttgagttacatatagatggtttactcttttgggtcttgctttataatttgtcagatgggttggagcagtgcttagtttaggactaattttttttttggactaattattcctctttaggaataattaggtaccatgcttaggaggcaagaccatcctgagtactctacctaatgaaccagaaagtttgggttttccagtccgcctgctgagaacagtgactttctagccctgtgtgagcgctgagctctgctccttctaatcctttccaatgcttctttccctggcctcagggagttttctcacacacatatctctgctgagtactcgagagggaccttccccagatctccagagctctctctgtcttgttttctcttctctggtgctctgtcttatgaactgtggctgtcttggtctccttagattctcagcacctcttcaattcagagggttgcctgtccctcctccttgtgccacagcctaggaactctctcaaagcagcgagttggggcagccatagggctgacttagtctctcgtctcccagggatcactgtccttcattgctcatgtccagtgtcttgaggactctgggttttgtctgttttgttttttggtttgctttggttgtctcaggcaggagggtaaacccagtccctcaccctcattgtgctcagtagtggaagtctcactctattacattagatattagtatttgtagcagagccctggttccctggtacttggggagctcttgaaaggccagaaacagcatgctttctcaccttttccagggcttcagtttctggtgcacatcaagcattccatacacatttgttaaagtcctttgttagacaagtagtgattcacaggttctatttgtaattttttcagttaacatgtattgggtatctgctgggagctagtaaaaacaaaaagtggtgtgtgacaaattcaattctgacaagaacaaccttaaacacttagaatatactttgagcatatcagaattttaaaaatgtgtggcccttgagtatttgaaaccaacaagaatctattgcttattagtagaggatattttgttaaacaagtggagagagaggcattttcagtctaattggtgttggcttttagcagctgatggaaaccagttcgtgattagccaggcagtggtgaaacaggctgtgcattctgaatgcctaggtatctaggcattcagaatggtggcgctctttgagttagcatcttcttctttcttgattcttttttttttttttttgagatggactttcgctcttgttgcccaggtaacaactccagtgcaatggcgccatctcggctcactgtaacctctgcctccctggttcaagcgattctcctgcctcagcctctcaagtagctgggattacaggtgtgcgccaccacgcctggctaattttgtatttttggtagagatggggtttcactatattggtcaggctggtcttgaactcctgacctcaagtgatgcacctgcctcgatctcccaaaatgctgggattacaggcgtgagccaccactcccagccccttcttgattcttgaaaaggacattgggtgctgtacatctcgttatagatgttgataaaaatgcttgtgagaagagtaacattaaggtagttatttggtcatttttgcagattattttaagacaattctaggactgatttgtggtaaatcacacattgctgtatcatagttgtgttcactgaacatattcaggggctctacagatgcagggctcttagctgctttgcacacttctgaattcctgccctgcgaacaggactggatacctaatagacaacaggtacttgataacagtttattgaattaatgagtgaatgaacagatacataaatgcatgaaagaatggttgtaatgtatataacttggatttcaagactttttactgactgttcaaaataagaaattgaaaactttcctctgattttcctctactatttacacaatttaaatggaagttatcttgtaccttcaatttctgtctaggattcgtacaataacgggtcatctctgagtcgcttaatgtctcacttgtctttctacagtgtgttgaagagatcctaggatacctgaaatcctgctttagtcgagaaccaatgatggcaactgtttgtgttcaacaagtaagagcttcattcttttcctcttctgttaagacgttcgggtatgacagcaaaacgctgctactccttaagaggcaggcgctgttggcataatcagctgggaggattgtggggtccagcgcagcactttttggctcagtccatgattgagccaagaggccatccttcccttcactccccaggaggacgaggtctgtcactgtggagggcagaggacaccagaagctcctctgcaacctcgctagttaacttccagtccctcggagtttctgtttagaatgctcaatctcatttagaattgcaaggaaacccaaaacgcctatttaaggtacaaacagcacttcatacaatatctcatgaggtattaatagtgattcacaggaagaatttcacgctgtgagtctttgctaacatatccagttatttacagatggatttgatatttgtgtgggagattcttaaaagtgttgttcacgccacattgttgatgcctcatttttttcactgtagttgttgaagactctctttggcacaaacttggcctcccagtttgatggcttatcttccaaccccagcaagtcacaaggccgagcacagcgccttggctcctccagtgtgaggccaggcttgtaccactactgcttcatggccccgtacacccacttcacccaggccctcgctgacgccagcctgaggaacatggtgcaggcggagcaggagaacgacacctcggggtaacagttgtggcaagaatgctgtcgttggtggaagcacgaaagagcaagcaggaaatactttgtaaaagaataaaaacgaaaaatgttagcgaacatcttctaatagtctgctgtattcagagaactctaggagatatatatggttgatgcaaagatgatttaaggcatagcccggccttccaagaagtgtgtggccagtgagtgagatgggcttgggacttacacatctcagaggtgggggtagaggaggaggaacactgagtgggctgagaagcagccagctctcattgccaaagtgtgtcagcaaaccagaatgcagttcataatgtccccacccattcaaagcacaggacctgtagagtggtgtggcatgtgttggtggcacttttcaggcctgtaacaaggatgaaagaacagcttcatagcagcacagtagtgctggtgttcagaggtgtgtgaaggccatagaagcatcttggatatattaccttgtgttttgtcagctttatgactagaagtctcttttcacttaaatttgttttttttttttttgagacggagtcttgctctgtcgcccaggctggagtgcagtggtgcaatctcagctcactgcaagctctgcatcctgggttcatgccattctcctgcctcagcctcccgagtagctgggactacaggcgcctgccatcacgcctggctaacttttttttgtatttttagtagagacggggtttcaccatgttagccaggatggtctcgatctcctgacctcgtgatctgcccgtcccggcctcccaaagtgctgggattacaggcgtgagccaccgcgcccggcctcttttcacttaaatttatgtttgtgtttttaatgcctagtatacaggacttcttaaattgccttaagtatgaacaggtatttgagttgctaatctgtatagtagcaataatagaatcccttgtttttccttttataaatttagcgattaaatagctacaattaaaacactagagtcaggagtcaaggaaaatacccatgttccaggctgtatgttagtgatgtacttactatatattggagtttcaggagtaagtctgtttcaatgctttctgtaaccatttggggtattaataagcatgtgagtgtgtgcatgtttgggttaatttcatatatgtttcttagaagggatatcattgatgtaaatattttaaaggcttgtcctccaaaaaaatcatgtaatttcttctaaattactgatcttttaaatgaccttcacctttctctcaaatctcacttaagactgggctgagtagtcagtttcctgtagcagaaaaaagctcagacttgagtagccttctgcgagtgaggagacttgatggctgtcaggcagctgtaaactctaaatagagtgtcattatctgaagagggcgatgctgccacactgagtggcctttcaagttgtttctcaatctgacacgttctgatcgtgtgaatgtgaaattggtttgagcaggagtatatctgagtgcagaggagattatttaaagatattctcattctctgcttcccttttattcccatttggcagatggtttgatgtcctccagaaagtgtctacccagttgaagacaaacctcacgagtgtcacaaagaaccgtgcagataaggtaaatggtgccgtttgtggcatgtgaactcaggcgtgtcagtgctagagaggaaactggagctgagactttccaggtattttgcttgaagcttttagttgaaggcttacttatggattctttctttctttttttcttttttatagaatgctattcataatcacattcgtttgtttgaacctcttgttataaaagctttaaaacagtacacgactacaacatgtgtgcagttacagaagcaggttttagatttgctggcgcagctggttcagttacgggttaattactgtcttctggattcagatcaggtttgtcacttttatctttcatccatcatacctgttcctaatttagtacaaattaccctaaaagacactgaaatctactttaaagaaatgtggtctgcatgtttccctcatcagttgctgctgcttatctttttcatgcacctagctggtgcagaaggcctggggcatagccagcctcagcaagtcagcatccttgccccagctccctggactcaaggctaacctggggttggctgttagggatttccaaaggtttgtcccatccacttgcctcccctccaaaataagtttgaatttaaattgtgagatacaattaagatttattgtttggggaacatttttgcaaaatctagagttagtttaaacagattatcaattattaccataattgatcatctgcagtttcaagctatctaacaggttcacttacctctttaaaaaggaatggaatttagcaggacagtaactgagacccgtgctcctggagtccatgtgggagctgtgtggctctgcacaagcatttgcacgcttcccctcttgactgcattaccttcctcctatagttgctgtgggcaccagattctggctagtcctgtcccttcatgatgcacattttcctcaagattcgtcccagttaaatcactgcagatgaaactgccttttcatcgtcaaaatttaactgtcatttttgagccgtgatcttgggctactttcttatgtggggtaggaatatttgtgagttagaaatattacacttctctatttccttctagacgtaaatctgttaatcctgtcagcactgttactcacctgaaagggtctgtttccctaggagaactgagggcactcggtcaacactgattttccacagtgggtattggggtggtatctgcttgttttttttgttgttgttgtttgtttttttttgttttttttttgagatggagtctcgctctgtcacccaggctggagtgcaggggtgcgatctcggctcactgccagctccgcctcagaggttcacgccattctcctgcctcagcctcccgagtagctgggactacaggcacccaccactacgccaggctaattttttgtatttttagtagagacgaggtttcactgtgttagccaggatggtctccatctcctgacctcgtgatctgcccgcctcggcctcccaaagtgctgggatgacaggcgtgagccaccgcgcccggcctggggtctgcttttaatgaaggaggcatcaaggggtgggctttgcgttggcctgatgctttcatctttctttcacaaaacctgtccgaagaaaatccgtctaaatgggccattgctctcctcaggaaatagtcattgggaacttcttttcctttcctttgacactaggaggctgactggggagaagccctggtctatggctgtgggcagcaggggctgagaggagcaggctctcaggggggcacgggtaccccaagggaagccagagccctgatttgttccattctagtaagaacaaagactgctctggtttcatgtttgttctgattgcctttcatcaaccggtcccctttctcccagttcttaagattcagtacagtgacagttttatgaacaagaatagaacactagaacagacaaaccattgaactctatgctgataaagatttattgagctcctgctgtatgtttgcattctgcccagaggctctgagaaaaccaggccatatgctccatgctttatccatggaagctccccgtcaggttgggaaagctgacagctgcagggaatacagtgtgacacaaaactggctcccatgcagcccttacgtgtcgcctctcagatggttgggggacgaaggtcgactcctttgggtatcttattactaaaccagtttcagggaatctgtgccaccctatctgccattaacgtgaacagatgagtccccaaggtgtaattttgggtattgtctgatgtctcttggaatttattatttgtttttccaatgagatttcacctcagggtatagtaaagttgttgaggggattcctggatgtgttctgcaattatctaggctgatttcagaatagagttatgcttatagtcaaatttatcagctgtcaagaattttatttaaaatttatgcagataagcaggaggaaaagaagcctggtttttacattttaatcctattattgatgtgaaattttattttccttcctgtaggtgtttattggctttgtattgaaacagtttgaatacattgaagtgggccagttcaggtaatagcattttattattttagatttttttcttcttcttgtgtacttacatgtaatttaggttattaagtgaatgtttaaactactgttaggcatttttgctgttttctttaaatggaaatctgactaacatactgtgcatttttgcttctcttaaaaattaatgtatatctcaagacttgtttggaagtagttatgtatctgaaaattccatatgttgtcagtattcattgcacatttcaaagcatttaattgtgttgacagatggtggaatgaaatcttgtggtggagcactagtttttaaatcttcttagagaaagcagttttatataatgttgtctttagtaattattatgcatttgtattctctgcagctttttcttgctagatgttgaggttttaatacttcttgctagtccattacaggtttataattattaaaagttaaaattcttttagtacctaaaatgcttaataaacattgtaattaggaaaatttagtgcagaaggaaagtgttcccagattccctggggtctggaaacatagtgtttattctaattacatgacacctccactgtgttttggggcaagttactgtttctcttttgagtttcaatttcttcaagagcaaagaggcagaggagagctaggaagatcgtagctgctgtgcccctgtgccgtcgggtgccttctacctgctgcctccgaacctttacacatgtccctgctctgcgcgagggcacagatgggatgcactgtggcaggggtggggttagagtagatcacggacacctgttagcttgatgtgtgcttgctgtcaaggttgaatcatgaattattttatgttgcttatattgatatgtatcttaattttaaaagaaaggtctaaatggatgtttttgtttttagggaatcagaggcaatcattccaaacatctttttcttcttggtattactatcttatgaacgctatcattcaaaacagatcattggaattcctaaaatcattcagctctgtgatggcatcatggccagtggaaggaaggctgtgacacatggtaacgggacacacctttcactgtcgtcttcggtgtcgtgatgtgcttggcagtgttcgttttcatatacccactttgaacgttgtcagtggcagccatgtgcttctcaggctctgcatgtgtgtctgtgtatgtgaaggtactggttagagacgtttcaaaagagaagagagcatattctttactctcagcaatttgtaatcttctcagggaaaaaaattcaagaaacagtaagataacctaaggtacagatagattctgaatataaagttcctgttcattcacatgaaacgctaaaagttcttcacttgatcttagccaaaaggccaagaagcgatgcaacactaaaaattcttaaatcgaacttgccgtgaattaaattttgatctctcatccagtggtattggagatatagtttgacttgggttcagggctttctgttttgcctgatgattttgctggagcttaaataaggaacccaggagatggccagctgtgcaagcccccagcctgtggaaggagctagtgtggttttatgaatgagttgcaaatctttctttgagctttttgaactgatcttccagcattgccctattgacccctccctgactcctttgctggaatctgtaggcttttgaactttgacagggacacatcctaagacccttgcaaactcccagatgtgagaatggcactactacttagagtcttttcgactcagcgtgtgtgcagaagagcatcaaccgggctgtgttgcgaggcagggccttggctgacctctcagtgtttacatagctaagccagttagtgtttgccacggcctcacaagggcttcagattcacacagccaaagtatagattattaaaggcataggtgtttggtttcctggacttggagggtctttggacagaaaatcagtaggcaaccacacccagtactttgtgctgggaagcttggtcatctgtgagagggtcagagagtatacccatgcgtgcatgccaccgaagggtcagtgagtattcctgtgtgtgcatgtctcagggccggagagagtatgtgtcactgagaggtcagagtgtttgtgtgtgtgtcaaagagggttgcattgtgcccttcactgaggggtcagagggtgcctcgcgtgtgtgtgtgtgtacgtgtgtgtgtgtcactgaggggtcagagtgtgcctgtgtgtgtgcttgtgtgtgcgtacatgtcactgaggggtcagagtgtgcctctgtgtgtgtgctcatgtgtgtgcatacgtgtcactgaggggtcagagtgtgcctctgtgtgtgctcatttgtgagcgtatgtgtcactgagggggtcagagtgtgcctctgtgtgtgtgctcatgtgtgagcgtatgtgtcactgagggggtcagagtgtgcctctgtgtgtgtgctcatgtgtgagcgtatgtgtcactgaggggtcagtgttcctatgtgctcatgacattgagggtcagagtgtgcctgtgtgccaatgaaaggcatttcttatatttttttatatgtggtcatagtagaccagttaatttattttgactcctgtgttagaccaaaataagacttgggggaaagtcccttatctatctaatgacagagtgagtttacttaaaaaagcataataatccagtggctttgactaaatgtattatgtggaagtctttattgtcttttcagatgaatcaagtagattattcttgagaccaggaatgttgctgttttggttatttggaaagttttatcattttcaaattgacttttgaatttgagtcaccttttttcagaagtggtgttaaattataggagccctaggttttttttctttttttagaagtcatcacaaaatgatcagtgttcagaggaagagctttgaccttccacatggtataatgattgataaccttaattcatctcttaccataaaccaagtatgtgtaagggttttctttatttcttgaaagcattttgtagatgttgagagcagttttccaaatgtaatttccatgaaatgcctgataagggtacccttttgtccccacagccataccggctctgcagcccatagtccacgacctctttgtattaagaggaacaaataaagctgatgcaggaaaagagcttgaaacccaaaaagaggtggtggtgtcaatgttactgagactcatccagtaccatcaggtaagaggaatgtatgttggaactgtcgtggatactttattgacccgtgcagatggaaggaagtgccatgtggtaacgctcactgttaactgtgttactttgaaccaggtttgggctttctggggcctgggtagatgccggtgcagggggatggggagggaggcggggggtgggggggtgtggtggagttggggaggtgcagtggcaggaggtgttgttggtgtgtatcctttttttttttttgagatggagtctctctccgtcgcccaggctggagtgtggtggcacgatcttggctcattgcaagctccacctcccgggtttaagcaattctcctgcctccacctcccgagtagctgggattacaggcatgcaccaccatgcccagcaaatttttttttttgtatttttagtagagatggggtttcaccatgatggccaagctgtttcgaactcctgacctcaagtgatcctcctgccttggcctcccaaagtgctaggattacaggcgtgagccaccatgcccagcctggtgtttatctttaaagtgggcacagccacaggagttcacctgactcctggtctgagagtcacgagatcgttcaagatagtgaggccctcttttccaaaacgaggaccaaaaatcaattgacagtgttggtcaagatggtagaaaccttaaaatgatagaaatctcaactctgaaataaaaactttatttgtatatttatttaccactattttgacatagggctaaggtctttttctttgagctgatttctggttttgttttcttaaagtggcataagaattcaaagacattttgaggaaggctgagtgcagaaatctctctttttaaatgacttctcctttcttttaacttgcactgttgtctagccctcacttattttgtcaattctttttagctgtttgtctttgaatcttcataaagccatagcttttctcataagaagcagcactttctttgttcattcatattttaatgaacccctgtagtatttaattaaatacttaatgcctaattaaatcacataattgcaatgcaaaagtacatgtatcataaagaggtctgaaaatgagcaactggcaagcaggtggtggcaggcagagctgcttgggtgggtgggtgtcatggagaggagttcatcagccacatgttcagtgagctctggatatgtctgtttagaaatgatcactaataaacttgtgctcaaccatgtatacctctgggaagcaggtgctcttcagtagattgcctctgcagagaacacagaattgaagtgaatgtccacaaaggcaatgagccacctgcagaatagtttagtcaaggctgtgtttgaagtttgccaaagattaatatacatttgattttcatgttgtgccttttctctgattgtgaaatattacaaattctatacaaataacaatgatggcaaatcctcctgagcaaagtgtgcaccttgtatgtgccctagaggaacttgtgtttcgttctgattcccctacatttctcatgtcatagagtgggggttgcattagtgtccccctgtcctcgctgggatcacatctgtttggatcctagagtcttccagctgaactgggacaagtataacagacggacacgtaggggtggaaaggcgtctcttggcagcagactttctaattgtgcacgctcttataggtgttggagatgttcattcttgtcctgcagcagtgccacaaggagaatgaagacaagtggaagcgactgtctcgacagatagctgacatcatcctcccaatgttagccaaacagcaggtttgtccccgcagccttggcttgttgttgcatagtgatggtagcttaaggtccttgtgaaaggtgggtggctggaatcagctcttccttcagtcctaatctgtgccttgatagcagttctccgtgctagtcatgggacagctgacttcatttcttctcacaatgccatctcaggttggtattgcccacctactttacaggggggatcccacagctccgagaggttatggaggtgatcaggcagcacacagctttagagtgctggggtgagggcgggccaaggctaactctaaagcccgaacccttacctcctacactgcctcctgcattctggtcaacccagtgttttatttggtggttagatttttgtttttgttaccttactgcttgtaatttagcagttttcctttcctttcccttcctttcctttccgacagggtctcactctgtcacccaggctagagtgcagtcgtgtaatctcactgcaacaacctctgcctcccaggttcaaccaattctcccacctcagcctcctgagtagcaaggaccacaggtgtgcaccactacgcctggctagttttttgtatttttagtagagatgaggtctcgctgtgttgcccaggctggttttaaactcctgggcgcaagtgatccaccaaccttggcctgccaaagtgctggcattacaggtgtgagccacctcgcctggcctattcatcactaatcagaatttctatgatcaaatgacatgaatcattgtttccacaactgcagtggaaggaaatggcctggcagtgccagtttcagaagcagcctgcccccagtcaggcacaggccactgtgcccccagtgtagcagcacctctgtagctcacagagaagggtggtggggacctccttgaggcagctctgccagaaaatctcatgagctgcctggcacagcttgaggttgccttttaagtggactcagcaaatacatgtttgttcatcttgattatacacaataaacaactactctgtatagtacgagtagtccgtggtttttggcatttgatttaaacttagaggcatgtgatattgatgttactgccttcatgactgcacccccattctgatttcataatggaatgttatcttgagaccagttagacaacaggacagggatcttggcttctggtgagattgacagcagttttagtgtggtcagggtctccctgcctacagatggttttagaatggtgccctggaagctttatcccattcttttctgtgcgtaatctgagtagagtggagatcgaaggcctgaatacatagtaaatacctgacttaatatctgccgcaatggaaattgtgtgatacaacatttatgaaacgcttagtgcagcacctgccaggtagctcaccacaggtgcatgttgcattcagaagtagtgctagatactatcctgttactggcagtgcatacatcagtgatcaaagcagattaaagaaagaccccctgccttcttggagtgaagattttgttgggatgcgggtaaggggacagacaatagaaaagcaagtgagtgaagtctataccatggcggctgatcaggaacaccgtacagaagaatccaggagggaagagagttaggtggtgtctgcggtgggagtggcattgttcagctggtgatgagaagaagctttggtgatctggtgacatttgagtgaatttgcagaaaggaaagatacaagcctaggagatacctggggaaggaacattccaggcagagcaaatagcagtgcaaaggccctggcggggggcggacatgctgttagggtacaagcaatgagggtggaggagtggggcagccatggggagggaagggagtgaggcctggtggggtgaggccagtgtggaggagccttgagagggtttgcgctgatgtggtgtaggttttagcaggatcattcttattcctgagttgagaatagccttgagggggaggtgagggcagagcagggccacccatgtgagacccggcactggagtggaatggcccaagtcagcatcccttggcagcatgaaagcaaaaccagcaaggtttgctggtggcttagatgtggcatgtgagagagagcagggctttgggggtgatttcagggtgaggacagggtggctgtggacaaggtagggcagacattgggggcagcaggaggtcagagcctgtctggatgtagcagttgagaccccataggtgcctaatgaggtgaggccagcatcaggtgtatgagcctggagttgtcgagagactgtggggcagggggtcagcatctgagatgtccactcacagtggacccagactggctggagaggaggaggagcttgaataccgagcctgctgagtcccagctccaaggtcaggtaggtgaggggagccagtgctggggcagggggagtaggcaggtgtggggttcctaaagccaagattttttttaaggcattttgtgcaggagggcgacatctgctgtcagcaccttgggaacttggcccaggtttggcagcaccgagggcactgatgagtgcttttggaggagcaaagggagccaaaccctaatgggaatgtgttcctgaaaggacaggagagagacttgggaaaaggttttacttgaagagggaacggagaaatagggcagtagccagaggaggagaggagtcggcaatgggttaagttggcagaaatgaaggcctgtttacgcactgagggcagaagcaacagggaggatcagttcatgacacaggagacacaaatcgccgttgtggtgttcacagacatgggttaggattggctgcatggatgacagagcactgtgggttctcccagagttgctggggaggaggcagagttggtgagcacaggcgagggtccaggatgcaggaatcctggagctcaagtcagttgttcccttgttgtaagatgtggccagtgttgtgagcttcacatctgtgccttgaaaaacaccacatctgtttgcagagttgtttactatgtatacacactcagtagaaacaaaaattggaaacagtcagtgcccaccatcaataagtaatggttgaacacactgtggtataagcttagactattttagcttgggctattttgcatgattaaaaatgttctggccaggtgtggtggctcatgcctgtaatcccagcactttgggaggccaaggcaggcagattgcttgagctcaggagtttgagaccagcctgggcaacatggtgaaaccctgtctctactagaaatacaaaaagtagctgggtgtggtggtgtgcgcctgtagtcctggctaactcaggaggctgaggtgggaggatcacttgagcccattcgtgcgccactgcactcctggggcacagagtgagactctgttagaaagagagagagagaaagaagagagagggagggaggaaggaaggaaggaaataaatggaagaaatggaagggaggaaggggagggaggaaggaagaaaggaagttcagccagttgccttgggagttctccattgcactgggttaagtgagaagagcagagacgtttatgatttttcaaaacaactaaaacaaaacctctgtgggtgagggggcaaggatatggctataggaacatggggcagattaagaaagggatatacacacaccacttagcatttgttacaactgttgtgggagggatggagtgcagaaaaagaaaaaaaaaagtgcacaccatcccatgtatgtgtatacaaagggacgcttggaagactggtccccaaaatgttggtaatgattgtgtcagggtgctgcagtgctagttgattttttttcacacttttgtatatttgagtcttttacagaaagcatttattatttatgtaataaaaatctaaatgacaagatttctgttatgggaaaaatgtagctatacagtgttgttgtaaaaatgtttgcttggttcaccactgaacttaaaatgcttttaaatgagggaaggtgacgatgagatgattatgatgatttgcccttgagttacatagctggtgtacaggaagctgtcgtttcttttggcttacgtagaaatgtttgtggtgtctaattccacagatgcacattgactctcatgaagcccttggagtgttaaatacattatttgagattttggccccttcctccctccgtccggtagacatgcttttacggagtatgttcgtcactccaaacacaatggtgagtctctcgcctggctcagcagatgaatctggacggcttgttcaggctctgattactgggaccacccccagaatgtctgagtcagtcagtttgggtagggcttcttgagagtttgcttttttttttttttttttttttggtgtgggggtggtgcggaacagagtctcactctgtcgcccaggctggagtacagtgtcatgatctcggctcactgcaagctctgccttccagcttcacaccattctcctgcctcagcctcccgagttgctgggactacaagcgcccaccaccacgcccggctaatttttttgtatttttagtagagatggggtttcaccgtgttagccaggatggtcttgatctcctgacctcgtgacccgcccatctcagcctcccaaagtgctgggattacaggcgtgagccaccgcacccggcctttttattttttttggagatggagccttgctctgtcacccaggctggagtacagtggcgctacctcgactcactgcaacctccgcctcccgggttcaagcaattttcctgcctcagcctcccgagtagctgggactacaggtgcgtgccactgtgcccggctaattttttgtatttttagtagagacggggtttcactgtgttagccaggatggtcgcgatctcctgaccttgtgatccgcccgcctcggcctcccaaagtgttgggattacaggtggctctcgcaccaagccaagagtttgcatttttagcaaattcccaggtgaaactaatgcctgcttttctgggagcacactttgggactcagtgatagagaggtttattggtaggatagtaaaataggagttattttctttcacaaaattggcaattgggggaaatttaatcttccttttttcttcagctgtgacttatgtattatgtttattttaggcgtccgtgagcactgttcaactgtggatatcgggaattctggccattttgagggttctgatttcccagtcaactgaagatattgttctttctcgtattcaggagctctccttctctccgtatttaatctcctgtacagtaattaataggttaagagatggggacagtacttcaacgctagaagaacacagtgaagggaaacaaataaagaatttgccagaagaaacattttcaaggtatgctttctatctgagcctataactaacccatgccttttgggaagtcacgtgatgtttcacagtcagtaagtctggaataatacctggtcttgcttcacttctgagttgggtaaagaagtctgtatcagtgtaattttctaatccgtcctgcattatctatggctcttggttcatacctgtcttgaagttctgtcatgttctgtctcttgtcctcagtagagatgctacagcagtggctcgcctcaggcagggcagggcagtggggtggctgtcctgggggcaggcagtaggggcacgctgacgtcagggaagttgaaacccaagagaagccagtaaaagtgagtctcagattgtcaccatgtgctggcagttttacacgctgtcagtaataaaagtcttctccctgcagggcagcctgcctccaataaatacgtgtagtatcaaatcctgtcttccctcataaattgtttggaagctccccaaggacagtgatgaggcactcgtaagtgcttgctgcctagatgggtccctctccacctttgctagattctgagcattcactgagttagagctgcttctgcaaatgtgctgcttctgctaagtggctgtgacttcatgcagccttcacttggtttgtcatcagtggagatgccctgtgttgtcgaaggagataagcccagtaagcctgctgggcaccttttggtttgcaggttcagcaggcagcccatggctttccctgtgtcgcattgaagcagctggctaaaattgatgatacattaaattcctgtgacagatgatcagcttgtatttgtgtaatggtgtacagttcacaaagcttaaaaaaatgctacctgccatttcatcctcagtgaggaaggtgatacacagagagaccaagtgactgtgtccacggcgacggcgctctgcatttcactttagcggttaatgtactctacctatatttttactttatatttaccatatatcttttcatgtatacttggcgtaagtgctttatagtagtcacctaattcactgtcatcttttttgtttcttggaaggtttctattacaactggttggtattcttttagaagacattgttacaaaacagctgaaggtggaaatgagtgagcagcaacatactttctattgccaggaactaggcacactgctaatgtgtctgatccacatcttcaagtctggtaggtgaatcacattagtcttcctggagtgtctcgttccccattctgcactatacactctcagagtgtaggagctgtgctgcccggtagaaactctgccttgcccagtgtgccagttgaaaatatttgttgctgtaagagtacacctgataccatgtgacccagcagttccactcttgggtatatacccaaaagaatggaaagcagggtggtgaaaagatatttgcatgccagcattcatagcagcattattcacgatagctaaaatgtggaaccaactgaagtgtccctcgatggatgaatggataagcaaaatctggtgtatatttacagtggaatattattcagccttaaaaaaaggacattctgacacatgctacaacatgggtgacccttaaggacattatgctaaatgaaataagccagtcacaaaaggacaaatactatgtgattccacttacatgagggacctggagtagttaattcatagatatagaaagtagaatggtggttgccaggggctgcaggggaggggagttatttttacaagatgaagagagttattctagaaatgaatggtggtgatggttgtataacattatgaatgtacttaatgctactgaactgtacagttaaaaatagttaagaggaccaggtgtcatggctcatgcctgaaatccaagcactttgagaggccaaggcaggaggattgcttgagccaaggagtttgagaccagcctcagcaacatggtaggaccccatctgtacaaacaaactagccggggatagtggtgtgcatgtggtcccagctactcaggagactgaggctggaggatcgcttgagcccaggaggttaagtctctagtgagatgtgttcatgccactgcactccagcctcggctatagagtaagaccctgcctcaaaaaaacaaaacaaaacaagacaagagccaaaaatggttaagatgggccaatcacagtggcttatgcctgtaatcccaacactttgggaggtcaaggtaaaaggatcacttgaagccaggagcttgggaccagcctgagcaacatatcgagacccctatctctacaaagaaaatcaaaaactagctagatatggtgggcacatgcctgtagtcccagctacttgggaggctgaggtgggaggatctcttgagctcaggagttcgaggctgcagggagctattattgcactccagcctgggctacagaatgataccctgcctcttattaaaaaaaaatccaaaaaaaaaaaaaagtaaacctgagagcttcctcctcctgtgttaaatttggaggccaagatgtttttgttacttttacaaatgatcaaggacggtgaaggttgggcatggtagctcacacctgaaatcccagcactttgggaggctgaggcggggtgatcgcttgagcttgagaccagcctggacaacatagcaagagaccccatctccacaaaaataaaaaaataaaaaaaaatagccaggagtagtggcatgagcctgagcccaggaggtcaagctgtagtgagccatgatcatgccactgcactccagcctgggcgagatcgagaccatgtctctagagaaagaaaatgacaaggacagtgaacccaagaaagtcataagatgccagctgtgcagcaagcatggaaagcagccagtccaaattaggacagtgtgttttccaagaagaacgatcgtttgtaatgagaatgctttgctttaaataaatgactaaatagctagaagcctagttctaggggataggcacgtctttcttctctcaagaaaatagaaaggcaattctaatttctagtaacagcaaacagcattaagtcatggtccaaatatgaggcaaaccaaaatgtggcttgattgttcagcagttgatctgttggaagcccttgatattaaaaaggttctcctttaagcggcttaggagtcacgatcaaagacctatagaaagagatgccatccttctaggatccttggctctcttgggaactagattcagatagtcataatgtaaatactgcttgagctttctttctttctttctttctttctttttttttttgagacagagtttcactcttgttgcccatcctggagtgcaatggtgccatctcggctcaccgcaacctctgcctcccaggttcaagcaattctcctgcctcagcctcccgagtagctgggattacgggcatgcaccaccacgcctggctaattttttgtatttttagtagagacagggtttctccatgttgaggctggtctcgaactcctgacctcaggtgatccacccgcctcggcctcccaaagtgctgggattacaggtgtgagccaccgcacccggcccgagctttcatttttgaaatcaatgtatgactgaaacactgaagacttactgacttaattatggtttcagaacagaatgaaaatgtcttcggttctgatgaatataaaaggaaaactaaccaagttaatttggcaagtagatggtagagatagaggtggggagtggaaggggaactaaaatcttcacctagcattgttgggattatatggttacatcatctgaagttgacagaccaaaatatagaggcttcagaggtctccaaatagaactaaacatgtaattcagattgttaggaggtagtataaatgagctaaatctcatctttattacggtagagttaatgggtgatgtctaaagttgtctgaagtctataaatcatgacaaattatgatgtggtgattgtattcaacagtctttcagttgcagggataaaaccccagtttaaactagagtaagagaaagaatgtgttggtttaagctcctggaaagtgcaggcaagggtagttggtaggactgcatctagtgttgtaattctgtggtctgcattgtatatttatgcatctcagctctgctttcttcttttcatttatataatttttaaattttattttaaagatagggtctcactttgtcgcctaggctgaagtgcagtggcatgaagtgcagtgcgaggctcactctagcctcgaactcctgggctctagagttcttcctgcctcagccttctaagtagctgagacaataggcatgtaccaacatgcctggataggttttaaaatttttttgtagaaatggaagtcttgctgtgttgcccaggcgggtctttaactcttagcttcaggcgatcctcctgcctctgcctcccaaaatgctgaggttataggtgtcacccaccacgcccagtctcatctctgcttcctgtgttagttttgttctctggtgggctgttttcacatgaccgaagatgacctctagcaggctgtgttctcagcccctcaagtaggcctatgtgattggccttgcatgagtaatatgggtgaccataaacccctgaatgctctggtccacatgggccaaatgggagactggacagcattccattgatgaggaggtggggctggtctccgggagtaagggagaggagcacatgcagtaactgatggtctgctgcaagggatagcagcacagcagttagaattttggaggtaactaccagaactgaaaacagaaatgataacaagtagttgccttaaaaagggatgggagcagggtgcttttgtgatcaaagctcctttctcttactggatttttgtacacattttgcatacatatcttagagtaaaagatagcattttcagccttggtccatttgaggatactcttggcgtggcccgcctccatgctagcaggctctggttgtgccaagttcagttgagcatcctggctcttgcctgcacggaacttccagtcagtgcgtcagtatcacaagtcttgatatttcctatgaagaagaacagtagtgcagtgacagacgaaatgggtgggcaggcagaggcaggatttctgagggagagaagtagctagctttttgcagagaagagttccggcacccaagagagcagctgagagtacaggcaggcaggcaggatgccggtagggcccggccgcacggcgccacagaatcctggagaaaggggcctcttcatggcctctgcattcagctgctgtcaccctccgcacaggccatggccaaaatttaattttcatagtggactctagtttttgagccttacttgctattattgaaataattttcttgtttctttttaaagatcttcggattatgcttcactgaccactgtaataagtttaaagttgagaaaatatggcttgttaatgaatgataggtcaattttagtatgttggtcattttaatattttgccaccagttggtttggatttgatgccaggaggagacagcctcatttctaaggactagtcttgcctttgtgggataagggtggtgtgttctgtgtccttctacatgtccgagcgatctctgtgcagctcaaatgtggtcactgtcttattgcgctgatttcctctccttccatctcacaattgaggcaaaatattgttactgttgaagtgttgtccaataggacttccagcagagacaggatgtctgcactgtctaatttagttgcctttagccacatgtggtgttctgtacctgaaatgtggctggtctgattggatagcttaatttataattttatttaattttaattaacttaaatttaaacagctctgtgtggatagtggctcctgtatgagacagtgcaggtctgttgagaagcagctttactggtgggagtggagggcttggagagggcacgtgggtttcctgctggtatcttttgaccttatttaatctgcccaacatttgcaagtaagttgtgtgtgtgtgtatatataaatgtgtgtttctgtcttcttgtttcctttgactgcatttatttgaaagacactaggtggcagaattactgtatttgattggtttcaagataagagttgaaataattcatctcgtgtttttatataagtaaggtgtgtttagcatgtaaaattggtaatatgtattcacgtactgcttaaacaaaggctatgaattccacccataaaccgaaaatgaagacctttaaatttgtccatttcaggcgtgggtacttcttaaataatacctggttcaggaactagtcagaatggcacccttgactttttgtttcctgcttttcctcttgttgggagaggagggtattcatcccaaagtggtttgcctatttcacattccatctaggataagcagaatagccaagaaagatagctgtcctcctgtttacaacatttggggtaaccagcatccctctcttttggtccaagatagactggtttagaaacagatgatggcaccagaggcccaggaggtggaaacatcagctttgtttgttgtccatgtggctgaattagagctgtctggccttgtagcctcaacacggccttccagctttgctcaccgtgattttcaaggacacatcttgtgctcttccctgcctgccatccagactatacccagtcagggtggcaggagctgctgccccttcctccctgagtcctggtcgtgggtggtggagatgtgccatgacgctcacggaggcatgctcaccccttcctctgtggcagaggggatggctgcacgacagctcttccctgtcctttccaaagcgtctgtggttccactttttggggcaaagcaggaatactggaagagagagaaagtggtcctttctatagtaataaagttgacattgattcaagttcatgcttggggaaaggacagggctactaacaattataatgctgggagcaatggaattttctcatgggtatgtggtaggtttaattttaattatcccagttaattcttagaactgctctgtgaagtatttcccgctttgtgcttaagttctaaaagatcctgtgccaaaaccaagaatgaaaacccaagcattctttcttgcccatcgatctttctctcatcaggccacttcttgggttgatagtggtgagtgtagccgctgccactttcagaatacccaccatgggccccagtcactgtgtggcgtggagaagagatggttctctctgtgtcatagctgaacaagcccagcccagagaggtttctgccctaggagctctcgatggtggaattgggatgcgatcccacatcctgcctgttttgaaaacagcattctttatttccaattcctgcttccattgttccttttaatatttctttgtttagctcacaaaaacacggcttgcggagctgctgcgtgcagctgtagctgtttctctgggtgcagcctgcatccgccttcctgcccgcctcctttcctgcactgccatcgtggtctccgggcacttggtccctttctcttcccctgagtccctttggctcccctgtgccacccttgtgatccacaggctctgccttctttctgtctcagactgctgctcatcactactcgggaccctaggaagggaggttccaccgagaagcatcttctcatctcagccacgttctcagtgccactgttgtctttgttaggtaatggtagctactgtaacaaataaaccaacatttccatggcttcacaccagagaaggttgtttcttggttttatgacaatgtattgagggtgttcttggttcacggatggttttcctccatgtgggaattcggggacccaggctcctttccttcttttggttctgttctccaggccttcacatcctctgtgtctggttggggacaaggagagggaaggtaaagaaggctttgtggccttggataagtgacaggcatgcctttgctggtgttctctcgtggtgacaggtcacagccccaccctgtaaaaggggactgagagacgtcgtcctgctgcttcccagcagcagcactgtggtctctgatgtgttttctgtgaggataaaaacaggtgattccaggatgaggaaagtcagggaaacccttggaaggaggggaccaggcgggtgtcaccatgggattagtggtggcttcagaatgagctgcagcgagtgccatgccttctaaagcttttgctattctgatatgcccacaccatgcccagcaggtgtctgccttgctctccgcagagagagtgatgaatccttctcatgagcctctgtccagttgttcctccctccacctggaagggaccctgggttcctcataacatcccagcggaacaggggaccttctatcctgtccccaagttcatcctcatcctcctgccggcttcctggcccctcttatgtctgcttcctgacgccacatccttctggattctctggaattgaattttgcctttgatgcttatttaaaaatatccattgcaggccaggtgtggtggctcacacctgtaatcctgtgcactttgggaagccaaggtgggcagattgcttgagcccaggagtttgagattagcctgagcaacatgttgaaatcctgtttctatagaaaatacaaaaattagctgggcatggtggcgcacacctatactcccagctactcaggaacctgagacaggaggatcaattgagccccggaggccaaagctacagtgggctgtgatcgtgccactgtactccagtctggtcaaacagagtgagaccctgtctgaaaaaaaaaaaaaaatccattgcatacttcaccgtagcgaaacatgtatgtcttacctttcctttcctgcctgtagctgctcttttacacttaacagccacactaagccagccttaaatgaaaaacaaaccagcacttcctgtgccctcctgcttccttcatgaggggtccctccctctgtgtacactccattctcattgcccatggtggtttgtttccctcttgtttctcaagccatggcagcctgcctcttgccctctttactaaaaaggcctttgcagaggctgcctgtgttctttctttctaggtctctctcatcctaggccctccagcttgattctgtggagctgccctcttgtcactcagtagcttgtggggtcttctctgtctagccacttaattgattgtgttcctcgagttgctgtccatggtctctcgttactgttttctctgtgtttctgcctctctccttggccttggtaggtccatcccctttgtgaccttggctgttgctctcatggacaactttctcttgctggtccttgtagtcctggcatccagcttctcgacacgggacttgtcctgccagtacctcagacttgcacttaaaattgaactagcaccactgtcactctccagggcctcttcttgttaattagatcattagggatgttcagaatcccagcatcatagtatgttcctcctcccgctaccccaggaaccctaaccttacctcctcctctctatctactaggaggtggccctcagagtccgtctcatcttccacctgaacttccctaataggctccagcagctgccaccccgggggctgagtacttcctccatgccttgtgcagtgctgagccctttacctgggttctcctgtttgctccttattacagccctgcgaacagatactgctcttaattccatcttacacctaaggaagctgaggccccaggtaaggtgcatccaaggtcacccaggtagtagacagtagagccacgatctgaaccaggcagtctgattcagagcctgtgttgacactcagccacctagaacacagcttggattgtgggtttctattacctgttcaaaacccctacatcccgggtctgtccctgcacgtgctctgtggcctggctgcatcttccttgaaggcagtgcatgcctcttcactcagggggcccatgcaggaacagagggccccacagaaggatgaggccagtgcagaatgggctggaggggacaatgctgaccaggaagcaagtgtagagaaatcccaggaaacctggaggagccagagacaaggcattagaactcctcgtcgtgacctggtctgcattctctgagtgtgctgcttctgttagctcgcttccttggtctcaggttatagtttaaggcattgtggagccctaaaaagcctgtactctgtttttacctgttttaggaccctttcactttggggatgtgttgattttttttttttttttttttttttttttgagatagagtctcgctccattgcccaggctagagtgcagtggcacgatcttggccactgctgcccctgcctcctgggttcaagcaattcttgtgctcccgcctcccaaatacctgggattacaggcacccgccaccacactcggccaatttttgtatttttagtggagacagggttttaccatgttggtcaggctggtctcgaactcctgacctcaagtgatctgcccaccttggcctcccaaagtgctgtgattataggcgtgagccaccacacccggcctgaaatttaaatcagaaataaaattttgatcccaacagtgatgccaggcagcccagatctgggggagagggtggccttggccagctgggcctttctctgtttcccaagtcttgctgcctctccctgctgggctttgcagcctgtgcatgtctctgtgcctttgaccttgtttatccaaaggagaggatagaatgaagtcatgattcctggagccctgagaaggatgctgtggagaaatttgccggtagaatctagctgagtgtgttgctgaggtgccagcattgtgtgtggggaggctgaccgcttggcctgcctaggcccaggatgctccatggccgggcacagaggccacttggctgtcaggtgtcaggagcctgcagagggcacacagagcctggaccgcaggggggtcctgctttctcacctggcctccttcagcatttctgtccctcagtccttagcaagcccaggagctgttgagtttggcaggtgccgagtgctgttcctgcctgtgtagctgtggctcagtcctgtgggggccccgctgtggcccgagtgcagtgattcgaggcgctgagtgttccctgactccttctccaggagctgtgttcagactttcgcagctcttggcttggagctcctggagggcttggcattgccgaccaatgtggaggtcgacagtgagagaggaggaatgctagctttcttgaccagtccattaaataagtgggatattggccaggcacggcggctcacgccttaatcccagcactttgggaggctgaggcgggtggatcacgagctcaggagttcaagaccagcctggccaacatggtgaaaccccctctatactaaaaatacaaatattagctgggcgtggtggcaggcgcctgtaatcctagctacttgggaggctgaggcaggagaacagcttgaaaccggaaggtggagtttgcagtgagccaagattgcgccactgcactccaacctgggcaacaagagcaaaactctatctcaaaaaaaaaaaaaaaagtaggatatctgtttctgcttagaaaaatcagaattttctaaatgccaggtgttctgaatacgtaagtatgggagacgactcagcctgtttcatttttatgtaaaatcttcgcgtagccatgtggcactggaccgagatgaaagcaaagacatttctccttaactttgtttctaggaatgttccggagaatcacagcagctgccactaggctgttccgcagtgatggctgtggcggcagtttctacaccctggacagcttgaacttgcgggctcgttccatgatcaccacccacccggccctggtgctgctctggtgtcagatactgctgcttgtcaaccacaccgactaccgctggtgggcagaagtgcagcagaccccgaagtaggttcataatgccccacagcccagggcgccagcccagcaccctgtcctgagactcccagtaacctgagctttggccaccgttaaagcattttcattttccattttttgtgagggcttgtgaaatttctgctgcatattaatattcctttcatggacagcatattattgggacaaacatgcggtccagctaaaggcattcaaaatagcagttgctttctaaatgcgattttctttggcaggttctttgacaccattgcatcttgtgggatatgcttgtcatgctctgtggctcctactaagttctagtccttaaattggttccatagccagacatgttgcaatgtcttaacctcattataaagtaaatgtggttctggttatccttagataatgaagtaacagtgtagcaaatttcaaaacctcttggaaatgttattttaccattcaaaaaggcttactaaggttctcgttatgggtggccctctttttgcaaaaggttttcaggcttaagctccatttctaggtgctccaacactccattatttgtatatgtatggaaataaaagctgtgaccacccccaaccctggcccccgcccagctgaatcctcagcacagtatttctggaaggctcaagatcccacgctggggaaaagaagttctggagacaaaagagggcaggtgctgccgtgcctctctgctcagtatggatactggaccttgtgctgccagggctcccagtagggccagttcatggcactcagctggaaagtccactgttgggaggcattcttaaccatccactctgtgccgtatgtagtggggtctggtcattctgttggaggagacagaccagtgacgacatttgaaatgcttggtggatgtcttaggcctgttacgatgactgagcactgtgggggcaggagacagaaagtcagtgtctcctagttctgtgctgctttaacgtgcatagaaatcagctgcggattcagcagatcactccttttctgacagatgggcctgcttactctgatgttatatcagaaagctctgaatctgggaattgtgtcccctgaattggagtaacagaaatgcttagatgatgagtgtttaaaagaaataaaccaaaggtaaatttagtttggaattcagcaagcgtcttcattcagccctctgagggcaaactacagctttttgtaaatgtaggtaaattctgtgactgtttcgtgaccccctctgatccagttttcctttataaccttctgtattgttccttctattatcctgaaataacattaatagattaggctgggcgtggtggctcatgcctataatcccagcaccttgggaagccaaggcgggcagatcacctgaggccaggacttcgagaccagcctggccaacatgatgaaatgctgtctctactgaaaataacaaaaattagccgagcatggtgacaggtgcctgtagtccctgctactcagaaggctgaggcgggagaatcgcttgaacctaggaggaaaaggttgcagtgagctgagatcgcgccactgcactctagcctgggtgacagagtgagactccatctcaaaaaaaaaaaaaaaaaaaaaaaattaatggatcaatggatttttaacctaataattaaatttcaaaaaatatcgttctttaatggtaatgtaaaggtaaaattaagataatatgtaacaagcatgtgagtgtctaaggtgtccccgtggtggaaggaaaaaataaatccccataagtgtccaagatgcccatagagagcagagctgttctggtttaaacccctgctcttagcactgtgtttttccagctgtgggtggtgggggatgagtatctttttatttccatgagatgagaaaaatgaattactagaagtgtgaaatacaaaacacagctgctctttttttagccatagactcagcagccataaaattgctgtatccagttgcagaaattcctgctgcttactcttgaccctctctcggtttgtgtgcatctcctctcaggctggctcccagatgggagctggctccaggcgacactgggtgctctgctccaggaggtccttatgtgggtcctgccctagcctagcccctctcttatggactctgtcactgtgggtttatgattcactctcaatctgtcttacctcttggtgaactgttagagtcctgcctatactttggcgcttgtgggtgtgttgtggtacacatgatgtgttggtcacttcccagctcatcttgttctgagtcaccctagatttgggacattcattcgccaccagtaccgggcggtgtatggcctgagatttgggggggcttgtgctgctacaaattggggctgaatttgagttgacagtggaccttctttatgtctactgctcatatttgaattgcaaatactgcctcttctctttcagaggctcattaccctatagctgtattattgcaaagtgcacaattacagcttgagtgtaagtcacactgcgctggcaggacggcccactgagaaagggcacgtttcctgttcgttagttttcacattgacacataatttacaatacagtaaaatgtacttttctatcaactgtagtcagtaacagcccccctcccccaaccacatcaagatatagaggagtgctgtcacttcaaacagttccctcttcctctgccacatcctgcccctccccaggtctaaccaccaatccgtgctctgtccctctgttcagcccattgcagaaggccatagaaatagaatctataggctaggtgtggtggctcatgcctgtaatcccagtattttgagaggctgaagtgggaggatgacttgaggctgggagttcaagactagcctgggctgcctagcaagaccccatctccagaaaaaaaaaatttaaaaattacaatcacgtccctgtagttcagctgcttgggaggctgaggcaggaggatcacttgagctcaggagttagaggttacagtgagctatgatcgtgccactgtgctccagcctaggtgacacagcaagacgttgtctctggggaaaaaagaaagaaacggaaccacgcggtgtgcagccttctgagtctggcccctttcggtgagcagtgtctaaagttctgtcgcgtgttgcccacgcgtcggtggctcgctccttgcaactgctgagcattgtatggctaggctgtagtttgttttcacttcaccagttgggaaacagagaaaaggcactttttaaaaagtttaaatctgtagaattttggtttttaccagttctcttctaaatcctgagggattacaggaaaagttgttgtatttcagaatattcttagcttgatgtgacctctgtccccgttaaggccctttgccgcaatgggaaggacgtcgctcggtcagaccctgaaggtcagaggggcagtttgggagtgtgtcaacattttaactgtatggactagagccaagagtctcaaggtttataattcccacgtattcaaaaagaaaaaaacaataaagtgagaagtcagtgtagagtgaaataacctgtgttagtggggaagaagtgtttttaaacaggatttccataacgtataacatcaacatgtttagagtggtgatgtttcattgggaaacgaacagtaaaacatgaaagcagggaggttttcattctggcagttggcaactttcacggcagatggagaatttcaaaagcaattgctcaattatcaaacatagccagtgtgagttctgaaataaaggtgctgattgaatgtgcagctttatggtggattttgctattcaggcaagcattttaattttctgcctgttaaattctgttttctttagtttttcatatgtggtttattgtagcttaggaatagataactgagagtatatattacacatacaacattctgatatggcaatatttaaaacaacttgtctgttttagaactagaattaaacataatcatcttcagtattttgcaaataagctcactgccatccagaaacattgtcaatgcatctgttgctccttctagaagacacagtctgtccagcacaaagttacttagtccccagatgtctggagaagaggaggattctgacttggcagccaaacttggaatgtgcaatagagaaatagtacgaagaggggctctcattctcttctgtgattatgtcgtaagtttgaaatgcctgtaaacggggttgagggaggtggggaccaggagaacatcctgtgtagatgacacttgcatggaccctctggaacccagaccgcccggtgtcctgccaagctccatcgaaactaaatctagaatgaatgtttacttctgctgtgacatataattggagaccaggcctggccttccagtcactggattctaagttggactgtgagagtttttgcagctgactcatttatcaaatgcccggctattggctcacgcctacatgatgctgggtatgtttgttaatttgagggaagcaatggaataataataactaatgatttaaaaaacaaagtaagtgcattgactgtagtggggttctgattttaaatttttttaaaaattaataccaggagcagtggcttatgcctaaattccagcaactcgagaggctgaggtaggaagatcacttgagcccaggagtttgagacaagcctgggctatggtgtgagacacccatctctaaaaaaataaaaaataaaaaattatccaagtgtggtggctcgtgcctgtaatcacagctctttgagaagctgagggcggaggatggcttgagcctgggagttcgagaccagcctggcaacacagagaaaccctgcctctaccaaaaaaagaaagagaggaagaaagaaaaattagcctggcgtggtggtgcatgcctgtggtcccagccacctgagagactgagaagggaggattgcttgagcccagaagtttgaggctgcagtgagctgtgactgtgtcactgcactccggcctgggtgacaaggcgagacccctgctctaaaataatttttttaagttaatttgtagaaaaggtgttagatgttctttgtcacattttatgatggattcctgtttaaatgccgttctctttaaagaaaaaaaaataacttgtgggagtttttaaccataaaactagcatcacatatttaccatggagaatttacaaaaaaacaaataaacggaggaaaataaaacctcctgtaatcatactactcagagataacttgctgttagattttggtctagatttaatactttttctatatttatattaaaaatatttaaaacatatgcatttctttgtcacaaacatggtatcttatagatactactgtcacatagcaaaacagtgttaaatattctgaatcagaaaaggaagccgactctccaactgaaagaggtgttatcctagagactttttctggtgatgacaatttattaatagtcactttttgctttactttctctattgaagtagtttttctattttgttctacttttaaggataatataatttataatgctgtttttcacagaaatataagaaaaaagatactaattttataagttaataaagtttgatcatcccaaatccaaaaatctgaaatccaaaatgctccaaattctgaagctttttgagtgctgacattatgttcaaaggaaatgttcattggaaggtttcagattttcggatttagggagctcaacaaataagtataatgcacatatttcaaaacctgaaaaaaatcctaaattcagaatacttctgatcccaaacatttcagataagggttattcaacctgtactgtcagatgatcccaaatgaaaaatattaatcgttaaccaaatatcaaggaattgatcacattttacagtttctgcctaggattatgaatcaagatgaaaaggctctgcatgtttaaaaatatatatttttattttcttataaatcttaaatatctacacttaagatttatttgatatgtgggatccattcatattttggattcaacagttctgtcaaaactgtggcagtgataggggattctttttttcccactgaactatcacaaaattggaaaaagagtaattggagaaccccactggcttagccggcccgaagcccgggagagggcaggcagtgctgtggatggggtcatcccagcgcaacgctgcccctgctacctgcggatctcgctgaggcctgcctttgtcctttgacccttggccatttgttagtgtctctgagagctggactgctgtaccctacttccccagggggcctaacttcacacagcctctgccgcagtgcgtggttggaggtgacggccttggtaaatcgagtttcctacctcctcaattatttgtgctcatacactgtatatttttagtgaggtttatatttgggatgtgttttctccttcttaccctttctggcctttctatggcattaatacctggtctcttcttgtgtacttgaaaatgaatctctcatcatatttttccttagtgtcagaacctccatgactccgagcacttaacgtggctcattgtaaatcacattcaagatctgatcagcctttcccacgagcctccagtacaggacttcatcagtgccgttcatcggaactctgctgccagcggcctgttcatccaggcaattcagtctcgttgtgaaaacctttcaactgtacgtcttcatcctgccgactattgccagttgcagttttccctgccttaaaaatggagtattgaaatttttaactttaatttctgatttgcaaaatagtcatcttttgttcttttccttcttgctgttagccaaccatgctgaagaaaactcttcagtgcttggaggggatccatctcagccagtcgggagctgtgctcacgctgtatgtggacaggcttctgtgcacccctttccgtgtgctggctcgcatggtcgacatccttgcttgtcgccgggtagaaatgcttctggctgcaaatttacaggtattgggaagagaaaccctgatattgatttatattgaaaatttagcaggccaagcaaaacaggtggctggctttttcctccgtaagtatggtcttgacatggtcaccgatagaaacatggaaacatctgcaaacttgccgttactcgtgtgtccgatctgactgtttcttgtatttttttctagtctgcccttactaggatgaactgtacacatcagttcatcctttttaaatgagcatgaggttattttgggttgttaggtgttacaaacacactaatgtgtttttgtctattagagcagcatggcccagttgccaatggaagaactcaacagaatccaggaataccttcagagcagcgggctcgctcagaggtaatgctggaaacacaggtcgtccttgtgttaggacaacccaggatataaaggatatagatttgtacgggaataaattcacaggacaagaaatcgatgtgccttataggtgggtttactgcagaagtgccataatagaaccttcctacttttaaaacaaccagatctcactttctaaagagtaaaggatgaccggcaggatcacgtctgtgacgtgagtggaggcagtttgcactcctggtggctgtttgagaggtagcatttagaatgcctgtattcactgtcctgtgatgagtgggaaaataggttatcaggtttatcttagcaaaatcaaagcatgtcatctaattgctaaacaagagttggcaaatctgagagacattactcaatccttggcatgcaggacttacatctgcatcctgttgccattttatgtcttcaaagcatttaatcatttagttgtgtttgcaaagtctttgagaagcctttgtcagaaatccctacatctcctatgtgagtgtatttccatgactgcagaataagttaaacttttacctttttccttcccttgcggggcggggtggggggcagggattgtgtgtgtgagagggagagagagacagcagagaaggagaatataattatcatgctgtgtactttgagctgaaactgcaaaaaaggaaaaacacacaaaaattattatgcttttcagtctttagagtaccttgtctattatgcttttcagtctttagagtaccttgttgatggtgtttttaaatgggattgggcacaattaggtggacagtttgggatgatttttcagtctgtagggccaagctcttttgtaatttgcattatgaagttgtcactctcatagcagatggcgggagataaactattattactttttgaccctagacttagtcttcagtccagatgagggagattaaaagattataaatatcttgtgccagatgaggtgattttattttgaaatgaccatgaattcctatcagttgtcttactgggatatttgatagtggaatttgtgcatttgagtcttagatgatctgttttacatttattaagaaagcctttattagcttttatactgtgtattgcctgttgcagtgtttgagtataaatgaaatttctggaaaatattaatggagtacaaactgtgatacttaaaagtaaactagggcctgcatttgtatcatgacctgtttgagtattgatgagaagatagctgtgaagaaaaaggtttaaacaagtgtattttcctttaagaagccactaatagtgcatctccttagagtgtatatttctagaatcctagtgtgcagagtttagactaagactaaaaaaaaaaaaaaacaaattatactgtaatttcatttttatttgtattttagacaccaaaggctctattccctgctggacaggtttcgtctctccaccatgcaagactcacttagtccctctcctccagtctcttcccacccgctggacggggatgggcacgtgtcactggaaacagtgagtccggacaaagtaagtgtccagcgtgtctgcatgggaggcacagggcgctgagtgcctctgtcacctgtggcagatacagagagtgcagaggaggtgccgtggacccaaggagttctggcgctcggctcggctcagtgaagctgtggttagagacgtggggggccatcaaggtctgagggagccaagcagtgctgatgtgggacccttttggtaggagtgtggggtgagtagttagtgggtgaatcaaggaatagtcggccgtggcctgcaggcccctgactgcacaggccttcaagcacatgtcaatgccgttagcctccctccatctcctcataccttctggccacctgtgagttgcactgccactgccagccattctggtatgttgtcagcacctccactgctcatacctcatggttagggaccacctggagccttggtagagccttggtagagccttggtactctactttcctggacaaagttcagcttatgaatatgaatttagatttcaaaaaccagcagcccaagtataagaaagcgaaggttcagtcctgccttcttaggctctattcgctaagcacctgccctgccctggttgctggggagagatgagtaaagcagacaacccaggagaggatggcaaaggggccgctaacccttagtggtttagctatatttggaaggcctattggaagttcaccaggtgaagggggaggctgtgagggtgcccaggcaggtaacagaagtccaaaggggaaaacctgtggtgtggtgagccgtatagccacagcctgccggccggcagccctctcagcctagtgcggtgttcccaagcactggcctaggcctgtagctccagggatgtgaagtccccttgaacgccgcccatcatgttccccttatccatttttttcttcccaggactggtacgttcatcttgtcaaatcccagtgttggaccaggtcagattctgcactgctggaaggtgcagagctggtgaatcggattcctgctgaagatatgaatgccttcatgatgaactcggtacggggggagcagtggaggcaaggaatcctcagcttttcttgtgacttccaagtgggatttgtctcatcatcatgtgacccacttgttgacaacacatgttggggactccagtctgggcagggacgggatgtcggagagactccactctgaatggggccgggaagtggggaggactccatttcagatggggtcgggacatgggggttatgctgatcgagacagaaaagcacattgtttcagccacattagaatccacggaggtgttgttttgaaatccagctggccccaaggctgggtgtatggtttgggatgagaactatctggcctccactggaggaacaaacacaggatgttatcatctaagctccatggccaagacagaatggaagtcaaggttgcgtatttgccgtagacttcaacacagtgtcgtaatgcgtgacgtcaataacttgtttctagtgtcttggaagttgatctttagtcgtaaaagagacccttggatgcagcgagatttcctctactcacacctctgttagatgtagtgaggttcttcaccccccaaccccagatgtcagagggcaccctgcgcagagctaggaggccatgcaaagccttggtgtccctgtccctcacccgtgggcaggtcctgtgagcagtgggggggccacctcttgggtatggtgcagccatggcccaagcagggcttcttctcagacctactaggacgggagaaacctcctggtgctttagccctgcgttgatatgcagcaaatgggagggaagtgggcacctgggaggacaaatgcctgtagaggccgggagtgacggcaggtgttcatgaaaagagaccttgtggggagggcaacacaacagtgtgttctgatgtactgaagagctcaactgaaaacaacaggagaattagcccaaaatccatttactaaaattgtttatctttttttttttttttgagacaaagtctcgctgttgtcccccaggctggagtgcaatggcgctatcttggctcactgcaacctccgcctcctgggttcatacgattctcctgcctcagcctcccaaatagctggtattaacaggcatgcaccaccacgcccggctaatttttgtatttttagtagagacgggatttcaccatgttggccaggctggtctcaaactcctgacctcaggtgatccgcccacctcggcctcccaaagtgctgggattataggcctgagccaccacgcccggcctaaaattgtttatcttaagattcatgcagtgaaagctaacttactgagtgataaatttgcttagtgatctgtttattaggttttccaaatttgctaattgggctttgaacagctgtaaaagttctgactgtaaaagaaagcttcaacttttggcattcatgatgcttttctgagtattaaactaagatagatgttttacctgaaggatcggccaccaatctttaaatggctaaacaaaagggttgctaaaacataatccaaattgacataagaaataccatttttccaaccaaaattttggcattcatatggctacttttacgtatttcagctgcatttgaacatctttttcaaactttagggtggttggtgtatcactgaggtcttggatgacactttagctttgattttgtttttatgaattaaaattgtcataccaaaatttttatttcaagcaaatccaagagcataaaaaattaaaatattacttaaaatactaagagagaacagatatatattttactaagcatatgttgaatgaaattgttcaaatatttataacaggcatagagtagaattttcttaaaaatatttttgatggtataccaatttgtattttctcagaaacatttgccttattcttttttctgttgtgtttttcttacctgattgaaagctcataatctgttgttattgtttgttaacctttaatgctctgatttcaggagttcaacctaagcctgctagctccatgcttaagcctagggatgagtgaaatttctggtggccagaagagtgccctttttgaagcagcccgtgaggtgactctggcccgtgtgagcggcaccgtgcagcagctccctgctgtccatcatgtcttccagcccgagctgcctgcagagccggcggcctactggagcaagttgaatgatctgtttggtaattaaaattaaaatttatcttatttttaaaaagcattccagggccagtatagtactttgcaccaagtaaatgtacaataaaggcagtggatctaatacattgaaagcgtttacagaggtagctaaagagcagcacgggtgtcctcggctcagaatttcttcctgtgtgtttgccactttgccattcattgacatggtcatggacatagggctctaagcccttgaggaaggctgggccagacctcaggggagatgcagccccaaaccacgtgcagtcctgtggacggatgtgtagatgtgccactgaggaacaatgtcttgagctttcatcagattctcagagaattgcttgactgcctttcgaagttgatgcatctgtgctcacgtttgcacccacccacgaggtccttctgtttcaggggatgctgcactgtatcagtccctgcccactctggcccgggccctggcacagtacctggtggtggtctccaaactgcccagtcatttgcaccttcctcctgagaaagagaaggacattgtgaaattcgtggtggcaacccttgaggtaagaggcagctcgggagctcagtgttgctgtggggagggggcatggggctgacactgaagagggtaaagcagttttatttgaaaagcaagatctctgaccagtccagtcacttttccatctcagcctggcagtaagtcttgtcaccgtcaagttattgtagccatccttcaccctcacctcgccactcctcatggtggcctgtgaggtcagccaggtccccttctcatctgcacctaccatgttaggtggatcctaattttagagacatgaaaaataatcatctggaagtactttatgtcttaagttggcctggacatgtcagccaaggaatacttacttggtttgtgttagtgcttgtaattcgcccccagaatgtgtacacgttctggatgcattaaagtctggcctgtatccttaaagggccatcgctgtgctgcctgccctcagcaaggacacactttgcagacccacagaggctccgcctccacctcacaccaaagaaagggaggagtccaaagggcatcagtgccattactcacaaaatgataaatacacccttattctgaaccacgtggagtcatatggtttgtgatccctgtccttcaggtttcagcttagtggggaagtgggaaagtcagcgtgtgatcacagcacagggtgattgctgctgattatattatgtgcctgctgtatgcaggatgaaatactttatatgcgtcatcttatttgactctcacaaccccctgtgagataggctctgttactcccatttgacaggtgaggaaagcaaggcttagagaatttcagtgacttgcccaggtcctctgagctaggaagtagccattctggcatttgaacccaaggcctgctatccctagaacccacgctctcaaattcaacctatgacagaggcaagccctggtgctgtgggagccccaaggaagagcctctggcctggtggccacgtagcccaggagagatttctacaggagcccacagcgctgaaggagagagaggcagcagagtaagggggctttgtggcagagaggggactggcactttggggaataggtgggtcaggactgaatgtaatggagccatgtcagagctgtccttctggaagggcaagggcacctggacgcgctgcccctcagtgctttggacggttccacaactgtgattcacacggcttccccaaacgaaggtacacgagtgggcattctgtgactcggtacttccctttaggccctgtcctggcatttgatccatgagcagatcccgctgagtctggatctccaggcagggctggactgctgctgcctggccctgcagctgcctggcctctggagcgtggtctcctccacagagtttgtgacccacgcctgctccctcatctactgtgtgcacttcatcctggaggccggtgagtccccgtccatgaacggtgggttcctatcatagttcctgtctgcttcaccatgtttttattttgtgctgcctgtttgccaggtactaagctaggaattggggatggagaggtagataaaatatgcatcaggaagggctgggccccatctcttactctccaatatattggagtctacactggaatttaactggaatttgcttttttagtcattttatttagattttgaagtttcagctttcatcaaaaatacctctaaactttatgtctctgtgatctttggtcttagctgttttatgtatttagtcttatatgatcataagattaataacattacattcagaagattatttgttttctgtcagagttaaaatgtttgtttttatactgcattgtaatattaacgtactgtaaaataaaagtggcttgttcttttcaaggaacagtatcctcaacaagggtcattagccacaatttttaaaaaattggacgtcatagtttacatgttagagggcgttttgaagctttgtatttttaaattaaatgttatagagtgatgttttcatgtttcataattgttttcatctgtgcatttgtagccaacttgaaaacaaagatccagggattactacttaaaagccagacttcttggaggttatagtgatgattttgatagtatcttgagccgtctcataataacctcagggtgagagatggccaacaggagacagtcgagggacttagaaatctgaatgaaatctgaagttcaaatcttcagacatataccactaaccaagagattggtacctcagtctagtattgtctgtttgtctaaaattggttctaaggaatctaggctagtctgtctatccctttcaacttttgtgaggctgcacaaatgtaaaatgttgaataaaaagcactgatggaagtgtgtagaaattcttctctttgttctgttgtaattttagttgcagtgcagcctggagagcagcttcttagtccagaaagaaggacaaataccccaaaagccatcagcgaggaggaggaggaagtagatccaaacacacagagtaagtctcaggacccatttttttcttacatgttgttcctccaggacttaaaaatcattcacagagacgtgcaccgcggtgagtgtggactcctggaagcgcaccgtagctccgctgtgtcctgctgctcctccctagctgtcagggaggctgtagtccattgctttgccagctcttttgtttccgagtgaacaccttatccgtacacatgcggctgtctctgaccctacagaccagctgggatgccactgggggagcgctcccttccccccgcacttcccacactctgcagttattctgagatccttgagggcagggaacaggtttgtcttctttgtgttctcagaaattaatgctcggcctctggtcagcaagcaacaaccttttgttgagtgataatgaataaataaatgtttcccacatgagtattcagtaacctcagtgtcaggttcagccatctgttttggtggatatttaaaagaaaattccgcttttcctacagaaaaaaaaaaaaatccaaatcccagtgatttaagccagttatagacttagacatatactacggcttttcatgcactttcctcccaattctagagtaggtattttactaggaaaatggtggcagtgcctgttgggaggaagattctttggccaagtgtcttttgttcttgccagggcccctaggctgctggggtgcttcagcttctttagcccagtgtctggtggggaatggcccctgttgcctgtcccacagaggtgggggtgcctcacctggagcctgtccacacattttacacagcacgcttacctggagcatcaggcatcttttccatgctctgtggctcaggaaacacgccttttcaatcatgagtgcaccagtgcttttgggctttttctccccgcttttgtgcaatcctggttgtggatggagttttcctgtctttagtcttctgcatagtacttttctcttctggttcccggttcaaggttttgtaattagagaatgacccagaagcaatggcattttaatgcacagccaaggacttctctgaatttgtatctcaaacctctgtgggtccttcaggcttcagtttgtgatttcatgatttcttgttgctacctaaggaatatgaaaacacccacctccctactctgcatcttccagccgagtggcacctcaggctgtggatcctgtgcttctgtggtgaggataagaatagtgccaaccgtgtggattgaaatcaatcagttaatccctccatgtaaagcacctggaacggatgacagtcttgttatgaatactcaacaaatgctatcatgatttttagttagatttccattgctttaaaacagttgagacatcttggcggtttgagttagagcaacgggccctgaagtgggttctgtttgggtgaagatgattatgcttattccccatggccctctttaggcaagagtgggaagctttctttgtttttttaatcacctcgataggacgttacttcttaaaggtcatccaataaatattaataggccgggcgcggtggctcacgcctgtaatcccagcactttgggaggccgaggcgggcggatcacgaggtcaggagatcgagaccatcccagctaaaacggtgaaaccccgtctctactaaaaatacaaaaaattagccgggcgtagtggcgggcgcctgtagtcccagctacttgggaggctgaggcaggagaatggcgtgaacccgggaggcggagcttgcagtgagccgagatcccgccactgcactccagcctgggcgacagagcaagactccgtctcaaaaaaaaaaaaaaatattaataaagccaactcgttagcgtggggcttaattgcttaagtccaatgagaagtccttctctatcctaggaagttgcccaaactgtagaatctcgtggcctgtgggtaatagccacgtaatacacactcactgcctcaacaaatcatattttagtaggtatgatattctagactcaagacaccattctgtggatcttcccaagggtgtgaagtgtccacagcgtctgccttgggagtttccatgcccaccagaaccatgccccaagcccctcaagcactctgacctaggaaagccagtgaagcaaggatgacaacatggccctttgatactagctgagggacagacacaggtcctgggagaccagagaaagacgaggggcagaggaggtgtcctaaaggaagtctgaggctgaggagccacaggatggcttccagctgtcacaggctgctgctggccttatcacagagagtgggccagagggctgggaaccaaggccagagctcaggttcaggaccattccagcaatcccagcagaaaatggggagaattgtatggtataggcggatatgaaggtagaatctgcaggccttcagtggccaactcagagtctaagtggattccacagttacagcttgagcagctggttgtaggtcatgctttctacactgggcatataggatgtgttttttaaaaagtcctctcttaaccgttgcttgtttagatcctaagtatatcactgcagcctgtgagatggtggcagaaatggtggagtctctgcagtcggtgttggccttgggtcataaaaggaatagcggcgtgccggcgtttctcacgccattgctaaggaacatcatcatcagcctggcccgcctgccccttgtcaacagctacacacgtgtgcccccactggtgagtctgctcgttccttgcagaagaccaagtacggtgaaaggcaccggtaggccctgggctgggcacacgtgagagggcgggacagaatccccgcagcccagaggctgcctgctgtggttctggtgcccactgtggttctggtgccaggctgctttcctcaggcaccacgtgtggaggtcgctagtagaaatactgggttttctaaaatgaactgaggccctacatccctaagagattagtgttagacctgattctagagcaactagaccactttgcttaatagcagaccagaaaccacaccccctcgagtgagtgagattttcctttggagataattcatgtttttctacacagttttgcagttgtcttcagaattggtttaaagtaggtgttattgccaggcgcagtagctcatgcctgtaatcccagcactttgggaagccaaggtgggcggatcacttgaggtcaggatttcgagaccagcctggccaacatggtgaaaccccatctctactaaaaatataaaaattagccaggtgtggtggtgtacgcctgtaatcccagctactcaggagactgagacaggagaatcgcttgaacccaggaggcgaaggttgcagtaagccgagatcgcgccactgcactctagcctgggcaacagagcaagactccgtctcaaaaaaaaaaaaggtaggtgttattgatcagaacccttgtttcagataacatgaggagcttagcttgaggagagtgagggttgatggagggggactgacttctgcccagtgaaatggcatcatctcccaccagcccgctgaaataagatgatggggcctgttccttagggcctgcagcatcctcaggcaggaaagaaaggccgacctggcagggtgtgagccagcaggtgtaggtcagggagaatggagccaggtcccagggaagaggcttgtggctgcctgagaagggtgcgtgcctgcctgtgtgtgtgtgtgcacgtgtgtgtatgtatgctggagagtctagggaggcttgctccaaggacgcagtattgtttgatcctgagagataaggattctgccgcagggaatgaaggtattccagatggcgggcttattccgaagaagaggccagtgcctggcggtgctggaagcagttgcagaacagggagttgtaggctttcctgggaagagagcagcaggggtgctggagaagcaggccacacttgctgcatggggttgctctcggccccactcttggtgcacagcgagtcactgtgggttcattagcatctggttatgagacagtaactgctcctttggaggggctcgtggagaccatgcaggagggcacggtcttgaggtcatgccgtccagagcacacctgaggataggccaggacgggctgcacgctgtaggtaaaattcctccagcaagctcttcactggcattgaggagttccctgagtgcggtcatctggaaggcagctgtaacaggcactgcagtctctccctgggtgggtaccagagaggagcataggggagcataaccgatttaaagagagggctttcctgtggtgaggtaagagattagctggtcattatcatagagccccctctgcctttgtgcagatgggctgtgggaatcctggggttccgttgggtcctttgtcacctcactgaaggcatgtaagctgagctggccagaccgtgagctgatcctgccacttgaacagcatcaagcctgcctctggattcttctgtgcatggcacttgtctgagcacctcacgcacagagaactggacttcagagtttacagaaataagctgtatggttcattttcatgcctgcttgccaataaacatatctgagctgaacctcattgaacgcctgcctttattctagcacagcacctgctgtttgtgggcgaggggtgctgtctctaactcctgcctgcttctcccagcactccctgagtggggtgtgccagcagcctcaggatgaggacaggaagtgggagggcagagcagatttgggagggccacttgatggggaaggaagtcccaggaagcagttggagctgttttctgggggagaaggtgccagctctgggacagtgttggggtagtgaggagggagcccagtggagagaagtcgggcttcctgcttcctcacagtatgtctgtcctgactcaactcggatgatgtcacttccttttcatcttctcaggtgtggaagcttggatggtcacccaaaccgggaggggattttggcacagcattccctgagatccccgtggagttcctccaggaaaaggaagtctttaaggagttcatctaccgcatcaacacactaggtactcttggggcctctccttcaggtcaccattgtcggacatctaccgggaggaaatccagagcccccagtactgggatcttctcatttgactccagaaaagatttaagcatgataataatacaaacctatgtgaatacattttgcagtgttggcaaaactccttttatactgagaaaatagatcccagttcctgtgttttgtggcttgaatcccagctttgtgtattccgggcttgtttgaagtcaggaaaggttcatgtgtagtggacaacgtgagaccaaattctgccttagattttgcatttaggctaaacagtggcagcacttgtctcagaatgttttcttgtgttcaccagtctgatcctgttgtgtctcagtggtccattttctcatatgggaacaagcagacgggagcagatggagtcaggtttcttggcactcgccttccccagagcctagaggcagcatggggagaaagcaggcttggggctcagacagtcctggtctgcttccagccctcctacctgagcagcgcagggcaagtccgtctaacctctagagaccctcagttttgtcatatgtaaaatgggggtcgtgtctatttcatagaattgttgcagatttagaaattacatttctaaacaaatgttaccccttatttctaaataagtgtctaaatgaataagtcaccacttttgcccctatttgatggcaagaggtgtgatcttgtggtgggactgtaatcagtcagttctcagtgactgtgccctgctgtggtgtttcctggaatgttcctgtcttgtcctagaaagtctggcaggggcaccctgactccactgtccagtcctctccccagtccctcgggcttctgcagatttgaggcttgtttggatcccagaaggttgtggcaggagacaccttgcctctactttcccctttataattcaatgtccaaagagagccctgagcaggtacctcacgccagctgcctcacggagctcctcctcttcctggctgtgaggatcggtatcagtggcctcctgctctctcccccttgcctaacacgagcacctttgcttacttgggtgcccttgctcttgaactgcccatcggacgtgcgtgacccaagactgtgccgcagtccttgccttgtctgtgctcattttctttgttcatttttttccctgtaacgtaaattgttatatttgtctgtatctgtgtctgaatcagtcctgcacgctctccttctctctgtctcttgttctttctttaccccgtttatcacggggaccccgatgtccattgctctagttctcctgtcctaagcaccccatcccgtctctctggccttaccacaagtggcgtggctgcctcagacatcatgatggggacatgaagcacagctgtcagaaacaactgttcgttagatacactcgaatgcagctcatcaatagggatggagggtctgtcggatgtattttcactgaatccccgttcctaccttgatacactctttttaatctattcttctagacaggtcagaggaaccattactttgacttttaaatttttagcagctttattgaggtagaattcacatactacagatttcacccactctaagcggacagcttggtggccattagttttatccacagagttgtgcagccagctgcacagtctcagggctggactccagggaagattttagcccatttagtgagtggggcagaagtggccctggccctgcacgaggttgcctgcatgggcgtccctgccctgtccctgtgtctgctccactgggggttgaccaggctgccagggccgacttgggcctgtgccacctgcctctcatgtgtctcggacagtgcagccgatgtctatacttcggtttcctcaatgatgaaatggaggggatagtgttccccgcatcatagaactgtgtgaggtttaagggactcactgcccttggcgtggagccttctccaggggccgtgctgtgtcggcgtagctgtcagctctccgttacaggcttgagaagggttgacactctctcatgtaacatttatatttctaggctggaccagtcgtactcagtttgaagaaacttgggccaccctccttggtgtcctggtgacgcagcccctcgtgatggagcaggaggagagcccaccagaagtaaggccacaccctgtgctggttggcacatgggcagttatggccgcttgcaggcctttggtggggaataaaataaggcagcaagctggtgttctttttttctcttaccttatttttgaaagagtagctgaatggtgtcttgactgatattccagagcagggacaaagcctgctgaggtctgggggctgcgattaccaatggctggaatgcattttattacggtgcattccatgttaaggatcaatacgattgtgccctttctggaaaatatcttttagtttatcaatattcagaggagtgtaggttgaattaaaatgaaaaggcactttataaaggccatgagtagtacctggtttcatttttctaatgtcttgcagagattttatcaggcttcttgaagtgttcacgtacattacgctaacacgatattaataataactgtgctctggtacagcggagccagcagaatgggaagttgtggaatgcaggcccttgattctgatagaaggtgtggtttgaactcacagaaatgacagtttggagggtagacatatgtcacaagtcatcaagattgtctttaaattcatgcatagaagctaacagggtgtcataagcaaggcctgtaaaatgtatgagggaattcaaagataatttattaaaaagtaattcatgtttggagttttgtgcccaaaggagtccttgatttgaaaaatgggcttttgcccatcagattgtttcagggcccgtgtgtgcggaggccctgccttgtgccccgtgagctcagcctgacagaaatcctttggtagcacttaaggctcctcttcctcccattgaggcagggaagactctgggttctgcaggcagaggtggttgtgggtgtcttgctgctcttgttgacatgtgggctctccttccaggaagacacagagaggacccagatcaacgtcctggccgtgcaggccatcacctcactggtgctcagtgcaatgactgtgcctgtggccggcaacccagctgtaagctgcttggagcagcagccccggaacaagcctctgaaagctctcgacaccaggtttgcttgagttcccacgtgtctctgggacatagcaggtgctggggacagtgggttccccgctgaagcgtccagcagcttcaaccaggccgttttccttcattgctagaattgaaaacaccgtccgtgtggcctgtgcaggagatgcagacccaaaggtggcctcctggtcagtgagaagctggaaacgtgacaggaactgacgtggggttattgagcatttaggggaagacgttagcagagcaggaatgagcaggcaactagtagaacacccacttaagggctcacggacaggtgctcacttaggaagtgagtttcatttggtattacaccaggttcctttaggcaaagcggagggaaagttctggtgtttttcacttgtaagattttgaaggaaacaaaacactctttaccttttttctaaaatgtaggtttgggaggaagctgagcattatcagagggattgtggagcaagagattcaagcaatggtttcaaagagagagaatattgccacccatcatttatatcaggcatgggatcctgtcccttctctgtctccggctactacaggtacctgagggaaagggtgcgggggagcggttgtacttgggctagaatgagagaagactggcatgctcaccacaccagtgatgcgggaagacctgagtgtggtctgagttggaggctgtggtgctaaatacgctgcccctttcataagcaggagtcttagtcaggcccagggaggaagtaaaatctggaaatgaatgagaagcattctctcctgccagtcaagaaatgagaagcgaaagaattctcacgggctgtaagaccagcaggatttaaaagttgaattagttgcttatgttaagaactcaaccaagttcatctacacaagctgaatctccagcttttcctaagaaaccatgtgtggcagtggctgcagggcagggcacagctgggcctgagcaccccgctccctgcacctctcccctccctgggccctgcctgtcactgcccactctcccaccaagccttccggttgtgtgcctgccctatcacaggcatcggagcttgtcacctggtttaaaagaagagagttgtgtggggatttgggatgcacgtttttcactcaaaagtattttagcgtagagctctgtgattccgtagctatttaggagtttaagcaccttgaaggctttaattgcagaaagttctatgtggacgtgcaatgtgttatacgcagtgtctatgagactcaaatgtttattagggcgttgaagtaaactgagcacttggagggccatggatccagccttcaaggagctcataagtcaggaggacccaggagcaatgacctgtcatagaaggcagaaaagaggggcacagaggtgggtgggaggcatacacaggcagctcctggagctccaaggggagcaagtgcttccagggaagggggcgtggaggcccctttggaggaggcaagttgatctggggtctggcagagggttagctggggacatttagcgggaggctggtgcccgggaattggggggatgcccagcagaaagacatgaggaggctggcctggggcgtgggggggtgtgaaaggttaagtgggggcattatcctgctcccgctcctgccggctgtatctggtcagcctgggcaccgaggtggggttctggaaggcactgttcaccaaaatgcttatctgggtcccccagagagcttgcctgcctggactgtcggctcgcctgcaactgctgactcctaagcttttgcagctcagcccacaaccagttcctattcacagaggtgggagctgaggggtgacaagtgactgctgcagtcttatttgtcatagagaaaaagtgacagagtccagcttgcccactggccctgccagcttaactggttataaagtgacaaatccccaagacccacagggctctgcacaacctgggccctcctgccagtggcggcgagggcaggtggctcacggctgggtgcctgtctgggcaggagctgggctggtatggggtgggcctgcggccctgcccccctgtgcagatcaagactcagggtgctggtgttcacaggtgccctcatcagccacgagaagctgctgctacagatcaaccccgagcgggagctggggagcatgagctacaaactcggccaggtcagtctcgcgcccccgccgcctggcctctgtccgtttctgtcctcagactttggcgcttgacacacccaggagaaaagctcagtgcactttttaaatgaaaggaagttttccttttttttaaaaaaaaatttaatgttcattgtttttatctgttttattcctaggtcccgcaagcagaggaagcattagttttgtttttatttatgttctgtattccagaaagtagttaagagacctcacatgtagcgatagagatgtgtgtaagagacagtgagagggcgtgacttggacttaagcaaggaccgtgagacacaaaaaggggggtgaggacagagtggagtcagctgaaatgctcaggaggaagtagacgccatgaagggccatggtatggggggccgcaggcgtggccgtgagtgtccctggggccagctcttggggggctccctgagtgtccctgtccctgtggccagttctgggtgggagccccgtgtgcaggcagacagctcggccacttcctagcaggtcacattggtctgtgcttctgtttcctcctcagataagtgaagggattcaagggtctgggtgtggtggctaacacctgtaatctataacattttaggaggctgaggcaggaggcttacctgagctcaggaggttgaggctgcagtgagccatgattgcaccactgcactccagcctgggcaacagaccagtactctgtcccttaaaaaaaaatgtaaacagaaacgtagggccatttgcatatgatggcacatggcgtggagccctacaggtgtatgctgggcggggcccggctgtgctggccgacttgcacctttccctccaccccggtgctgtgtctttcgctcaccgggttcctgatttagtgaaagcagttgtgcaggacagttctctttgtagcttttgtttctgtggaaatgggtcagaatatggtgtttagaaacacttatgagctctgagagtttcctcttctgagttcctggcctgcagccttcacagcagaaaccctgtgatgtcacaagcctgtttctgttccctgctctctgcctgtactgtcctgttttgtgcctgccggtttcagtgacaggaagcagggagctactggaccagcctgtatttttctagacatagttggaaaaagaagtcccactcttctgtcctttcacctttgacagatgtttccaccccaagataagtgaaaatgaccaataggatgcactgtatttttcatgaaagtgtttctgaagggcaggctgagagtgagaggcctggggctcactgggtgcctctggccttgtcctgggcccagggacactggtctgtgcccgaggtattccctatccccccaaccccgctgcatttggccacatccttcaatgtttgcgttgtgtccagcgtccgcaaaccaactgtcatgggatcatactggggctgaagtacggtcccacccctgccctgtctggggctgaagtacagtgccacccctgccctgtctggggctgaaggacagtgccacccctgccctgtctggggctgaagtacagtgccacccctgccctgtctggggctgaaggacagtgccaccccttccctgtctggggctgaaggacagtgccacccctgccctgtctggggctgaaggacagtgccacccctgccctgtctggggctgaaggacagtgccacccctgccctgtctggggctgaaggacagtgccacccctgccctgtctggggctgaaggacagtgccacccctgccctgtctggggctgaaggacagtgccacccctgccctgtctggggctgaaggacagtgccacccctgccctgtctggggctgaaggacagtgccacccctgccctgtctggggctgaaggacagtgccacccctgccctgtctggggctgaaggacagtgccacccctgccctgtctggggctgaaggacagtgccacccctgccctgtctggggctgaaggacagtgccacccctgccctgtctggggctgaaggacagtgccacccctgccctgtctggggctgaaggacagtgccacccctgccctgtctgggatgtttagcccctagatgccactggactgagccgctacttgcttttgggaaagaggggtgggggttaggggtctgggcgaggggagtgcaggggctcctccttggcctgagagctgttcatacagactcctcgcccactccctgcagggtgctgggtcccaggggggaaatggcccttggtgccaagaacgtgagttggggctagtgccagtgatgatggagaacagctttttatgggcacacagcccacagcactgtgccaagtgctcgaggcttcccgagaaccaggcagaaaggaggacagtcgaggtgtgctgactgcgtggtggctgcgtgatctagagcgcgggtcacaaaggcgcgagggagctctggccttgggtttaccgcaatgactgccagtgcgggagactggaaaaggaatctcacgtattggttccgtgttttggggactccattcagatgtcacttaggagtgaaagcatcccttcgtagagcctctttctgtgtcaccctcctcagctgctcctggggttgactggcccctgattcatgcctttagcatgtgctggagcttcccagcagctgtccagcccctgccccaccctctctgtgggctcccttgcccgtaacctggggtgtctgaacgacccttgctaaggggcagactgttagacggtaggcatgtgctgagtcccagtggccacacccacccaccaggagcctggcactgtggccgcagcactgagcagtgccccgtttctgtggcaggtgtccatacactccgtgtggctggggaacagcatcacacccctgagggaggaggaatgggacgaggaagaggaggaggaggccgacgcccctgcaccttcgtcaccacccacgtctccagtcaactccaggttttccaatggcctttttctttttaacagaaatttgaaatttcttatcagtcatttgatttgtttgaggtgcttcttgaaatgagcctctcatctcatgtacttggaaaatacccatctcgcatattccacaggaaacaccgggctggagttgacatccactcctgttcgcagtttttgcttgagttgtacagccgctggatcctgccgtccagctcagccaggaggaccccggccatcctgatcagtgaggtggtcagatccgtaagtgagccttcccattcccctcacacctgcacgtgccacacgcaccacacacgccacacaccccacacacacacaccgcccacacacatgccacttgcacacacacccctcatgcatgcaacacacacacaggccacacgcaccatagacaccacacacacatgccacatgcacacacatacacggcatgcaccatacacacaacacacacagcacacatgccacacacacacgccacaccacatgcaccacacacatgccacatgcacacacactccacatgcatgcaccacacacacacacacacaccacacacaccacatgcaccacaccacacaggttacatgcacacaacacacacatgccacgtgcacacaccccacacaccacatgtatgtgccacacacagcacacaaccacacacatgcaccacacacatgccacatgtgcatgcaccagacacatggcacacactacacacacgccacgtgcacacaccccacacacatgtacgcaccacacacatgccacacacacatgcaccacacacatgccacatgtacacacatgtatatacacaccccacaccacacacacaccacttgcacaccacgcacacacaccacatgcgcacacacacaccacatacgccacatgtacacaccatacacacaccatacatgcaccacgtgtaccacgcacccacacagacacagcacacgcatacaccacacacacacgcacacatgcgtcccgcacagtaatgtctcttgggtgtaagaacacgacttgccagtagtagcgttctggatgcgttgcctggattctaacagcgcgattctccccttgccctcctggttttccacatctccagcttctagtggtctcagacttgttcaccgagcgcaaccagtttgagctgatgtatgtgacgctgacagaactgcgaagggtgcacccttcagaagacgagatcctcgctcagtacctggtgcctgccacctgcaaggcagctgccgtccttgggatggtaagtgacaggtggcacagaggtttctgtgctgaagccacgggggcccatctgccttgggacctggtgttggccagaggtgccgggtgcggctgcctccttccaagagttgacccgaaccggactccacggcccacgtgagctgcagtgcttctcagatggagggggttcagcgacggtcagtgccattcacaggtcactgtgatgtgggttgtggcggccaagccatggtttggggtcccgtatccctgggcttatgacatcattgtagtagcccatccccacagaaccacggtgtgtggtggcgctgaggcatcgtagatggtggaaatgctactggcttccccatgctctgccctgaggcctgactgcctcactccccttctcagttatgttccaggccccccgagcttcctggctggacagcttctctcctgggggccgttttgtcacagtgaccctgtgtttctagtcccaaatctgggtgctatagtctctttttagcgtggtggttgtcttagtcttttttggctgctaccacaagttaccttagactgggtaatttataaacagtggaaatttacttctcaccgttctgggggctggaagttttcatggtcaaggtgccagcagatttggtgtgtgatgagggctgctctctgcttcatagatggcatcttctggctgggtcctcacggtggaaggagtgaacaagctccctcaggccttttagaagggccccaatccacaagggctctcccatcatgacctcatcacctcccaaggccccaccttcttgtactgtggcactgcaaattaggtgtcagtgtaggagtttcaggagggatagaaacattcagaccatcccagcggtcaagtgttcatcctcttgagttcctccttattctgcttctggtttatcaggattcagccagtgcagcatggtacctgtattctgtggcacatcaccacatggtatttgccaagtatccatcacctgcacacgtgaaatcattgcccgtgggtcccgacatctggcgaagcatattcaaggatggcagaactgtcagagctggcacctctggttccttgtcatgtggcattacctagtaatccattttatgatagcaatggaaactcatttcttcaacaaacacctgagtggctgccgtgtgccagccgtctggggcccttggtgagaatggcatggtggtgcccatcagggcctgcctagcccgtgctctggacgggctcctgtgtgtcaggaacgacaatgctgtcatgacggtgaatgatttttttttttgccatcactccagccgctaacatttgcggagctcttcctcccgcacccccacctgacaaggccaagggtgaccttggccccaccctaggcggccaaggtcagaggttagctggcttgtctgggtcacacaaaatgcagcagaggttgaggtgagcacatgtccgtgacctggagcctgactccctctctgcgagtcttgactgctcttgcctagactctgtcctccccgagcccaaacgccagtcatcttcccttgtgggtgtccttcagcctggtgccatgctggtgactcagcagccgtccagggagtggaaacaattgagtgtgtgggttccctgtgtgggcatctctcttcacggcgaacaccctctgggtgttgcccacacgatgtcaaagcggctcttggaaggggtccttctcctttgtgggaagtttcagctgctgggctaacttgaattgtaactgtggttttgtgctcaggcccagatccccctaggcaagtgttgtgccatcagtaatcaaatgagaaataatcattttgaaaagcagatcctaaggcaggatggtcatggacactcactcccagctctttgtgcactcatgctttctggaagatggccatcctctgtgaaggttttcagcgcgtcatgcttggtacccacgtatccagagcatgtcgttttgaggtatttgcccaccgttgtgaaatccgtgccacccgagagcaggtcctgatgtggggctttcagaagtgggacctggggccgtacgcagtccttagggaggggccgtgtggcgttgtgcgtgtgaggggatagcacagggtgaggtgggggcccaagaaggaagtgacccacaaagaacagcctcctcttttggtccttgttcctgggatggctgggagtggcttctgtgtcgtccggccatttcccctgcggagaggctcctaccactgccgagaacctcatcattccacaaaaacaagaggccgcctggccatccagcgctccatgggaattctgtgtccccatagtcttgggctgaaggagggtgacattccttgctgacttctgcaggggtctcctcactgttaaagagcagattgaaagtgaagaacgtgggctaagtgtttaggtcgatatttaaccctgctaggttttggatactaagtgaaattgaggccattttggttgaagttgacagaaaccactatcagggatccccaagactaccccaggcttttctagaaagactctcagctaagatgtgttatggtaaaagcacacaaaacaaaatcagcaaagaaaattagcaagggcagaggcccatggggcgatgtcccgaggacaccaggcttgagcttccagaatcctctcccagcggggtcgtgcaggacgcacttaactccccgcacagtgagccgtgacagcgcgtgtgcagtgtcgtcgccaggaaagcacactagagactcggtgccagggtttttactgggggctgggcacatgggcaccctctgcctgcctcgtgcccagactctggactcccggagggaaggcaagttctcagcaccaaccctggtgcccacacaagcagctgagcacagggagcccctcctcagtgaggatggtgggcaccgtcccaacaccagccaggggccagccttgcacacaggcctctcaggatggtctccggcctgctgtgtagtctcttctgcacacaagcgtgagggcagcgcccccgcctcggctgtggggaggagccactgggacgtgagctctggtggcatgcagcagcttttgtctgtgtgtgcctaggacaaggccgtggcggagcctgtcagccgcctgctggagagcacgctcaggagcagccacctgcccagcagggttggagccctgcacggcgtcctctatgtgctggagtgcgacctgctggacgacactgccaagcagctcatcccggtcatcagcgactatctcctctccaacctgaaagggatcgcccagtgagtgggagcctggctggggctggggcgggggtctcagaatgagctgtgaaggaagcagcatcaccctctccaagtgcccaggctcctggccagatggcaggccaggtatcagtgggaacccaggtgggtgccatggctgaggtcagtgagacgcaagagcacaggtgcgtcctagaggcttcctcgggcacctccagcgagctggagctctcgcctctgctgctgtctcatgtggcgcttagcacactctcccacgtgcccattcctgactctgctctcgaggccatcggctctcattctctgctcccagaaccctgttattacccaggctagcctcctctctgcaccttccccgccctggcccagtacctccctcttgtttccactgtgattccgacctcaccttatcttaaagctgctggacggcaggttctgtacacacgtgtccttgacaaagcacggctggtgccgcaacccctcagcgagcaagtcaagctcttcacagcgatgtcttacaagcgcagagggctctgtgacaccctggtctcaccgccactcttccaaagtcgcagaggctttagcagagatgggcccagcctctctgagtcataggcttctgcacacgggagctgtctttagagggagggtggaatttcatcagccacccacatgggggagttgagggcaagaattaggagcaaagatgggaaggggtctgggaggaatggccagtgatcccctttgacaagtgggcaggaaacgggggctaggtcaaagttgagtggaagacctggagggagacgggaaggtctctgtaggcacagttcagacaggagggaggtgtgagccagggcacatgccggtggccgtctggcaggatttgggacatgctggagcagggacagcggctcatcaggggccattgccctcatccaggccagagtgtcacaagcccgtggggaggcccttctcgcctgtcatccttgctgggcagtgggtgctgtgctagcaggacaggcggacggctggcaactgtctctgcatccctggagcctggcatagggccaagtcacacggggcacaggcctgcaaatcaggcacatatgttggtgcagtgacgtgattttggggggcagccccagaacaggccccagacacaggccaaagccctgcctgtgctggtgtgttgggctgttctatggctcttgctgtgggcatggaggactcagggaaggagagttgaggtggtccaggagttgcgtttgggatgcagagagcttgtggcatccaggtagaaatggtgcgtggggctgacctcagcaccatgggcagaggggccgtgtcacgtgcctccgaggtggaggtgggaccacgtggtgacagatatacgcatcactgggcacgtttttgtgggtgttggggggcatcgtattggctcctctgttcacagtggccactcattcagtccctggctaccaggtcctcactgtgccatggggaaggccggcgctgtcgggggatcacagaaggcagcacgtcatgatggcatgtgccatgaaggaaaagcacagggcactcaggaagtagaggggactggcctggggtgtgggaatctagggcctcgttgagggacagagagaggaagtgtgtggtggccagcatggaggtggccacaggggaggctgagttaggccgagagggcagggcgttggggaggtagacgggctcagccactcagggagtggtcaagcagaggctgaagggtcaggccaggttgcaggggcctgggggagccactcagggtaggcgctcccgggagcccgcctggcccatagctctacactcccgcgtggggccggacatgctgtgaagccctctccacgttggatgggggtggctgagcctggatgctgtctcccgttttcagctgcgtgaacattcacagccagcagcacgtactggtcatgtgtgccactgcgttttacctcattgagaactatcctctggacgtagggccggaattttcagcatcaataatacaggtgagtgggccctggctgtcttcctctgcacacggggagtgggcttcccttctcttttccttgcaggatcataccagtgggccagttttgacttggtcgggaggaggcatgaacacctgagactgtgcagcgattctttgacacagaggcctttctccctgtgcagatgtgtggggtgatgctgtctggaagtgaggagtccaccccctccatcatttaccactgtgccctcagaggcctggagcgcctcctgctctctgagcagctctcccgcctggatgcagaatcgctggtcaagctgagtgtggacagagtgaacgtgcacagcccgcaccgggccatggcggctctgggcctgatgctcacctgcatgtacacaggtgagcatgtacacggtgcccataaggccagcccaagtcctgttcaagggaggcaggagcatgctcactcaagggacctcgactaggtgccctctgatttcacacttctggtgttgccccaagccggccccatcaccttgcaagaaaggctctggagcccccagggctggagtacctggtcagggttgaccgtccctgtggtcactcatcccatgtggctgagctgggctgggtcctgggcaagcaaggggctgatatcacctgctttcagatctccagggactcactggacccctgtgtacaaagcactgtctacagagcctattgggttgtatagaggtaaccttcgtactgaacacttttgttacaggaaaggagaaagtcagtccgggtagaacttcagaccctaatcctgcagcccccgacagcgagtcagtgattgttgctatggagcgggtatctgttctttttgataggtaagaagcgaagccccatccctcagccgttagcttccctagaactttggcctgaagctgtgcttttgtgtgtgtctgctgatcccctggcgctgttgctggagtcctgccagtgattccccaccacagcctgaccatgggctgccttggctcagggttccactggcgagctggtggtccttggaccccagcactcaggtgtagcgttgaccagttccaaggttgtcccagtgcctgcccatctctcctgagggctcagggacagtacctggcagttgggggtgtggcagggggcaggaatgaccagcctctgggagggtggggcagaagcctgtacagtgaggaggagctggctcagcctggctgcctatcgtgagaggggagcccacggggctgtgggaggggggccgtggtgcctgtgagcagggtgaggagcagcggcaggaggatgaaggtggaacccacacatgcatctttgagacccgtgtggtcagtggcttctgccccccaccaccccccactgctgtgcgtgcatagaattggcttccctcacctgctctggaagtgggttaggagcttggtagggctttttctcaaggacaagggcccctgatttgctctcaggcctcagtcctggcgacatggtggatctggagccttgttgcactgccttgcctgtgctctccaatcagggtggccagtggggagccatttggcttttctcaagagcatactcaggtggaccttgctccactgtttgaccagatgaggcattctgaacagccaagcctgtgctggtctgttttcatgttgatttttttttttcttttctttttgagatggagtttttcccttgtcacccaggctggagtgcaatggtgtgatctcggctcactgcaacctccgcctcccgggttcaagtgattctcctgcctcagcctccctagtagctgggattacaggcacacaccaccatgcccagctaatttttgtgtttttagtagagacggggtttcaccgtgttggctgggctggtctcgaactcctgaactcaagtgatccaccctccttggcctcccaaagtgctgggattgcaggcgtgagccactgcgcccggcccccatgtcgatttttaaatgcacctctgcatcgttcttcagtccccatatgctcactgagcaccactgcgactggcagacgggcacagggaggcgccacgaccagtcctggccttcaaggggcttgtggtctagtgggcccaatgctaggtggcgagtgctccaaagagtgtggtgcacgccttccgcttgaccgctctccagacgccacagggaggcacctcgcagctgaccacagatttctctctgtggagcagtgtcttcagagcggctgccatgccactgctgggcgagggtctgcgggcgggtagagccaggagcacctgtgaggaagtgcactgccattttcgtagctgcttcccgtgtgtctcagttacacacggctggcatgtgtgcactgatgagacgggaacgtgatggttgcttttcagcactgaaagggatactgctcagggggcgtgtttcaggatctggttagggaagaagcagcgagagcacagatggggccctgtgtggtaacaagaaaaaagtcctggttgacaacagtgccacgaagcgttagaacacatagggatgtttgtggagcatttgcatgtggaaagcagcaaaaacataatgggaacgggttcttttgttatgatttttaaaaatctcttttgtaacatccttcccgctgcgccgtttctgcatattcctttatgtagctttcaaactcctcttaggagttctggtccctacagggcgtgggagcccaggctttacgtagctttcaaactcctcttaggagttctggtccctacagggtgtgggagcccagggcctgtgccgagcagcctgcctccacgagctagacagaggaagggctggggttttgcctttttagtctcaaaattcgtactccagttgcttaggctctgactttccccacttggaaagtccctcacggccgagggtccctcccagccctgatttcacatcggcattttccccagtattagagccaaggccctccgcgggcaggtggggcagctgtgggagctggtgccagtctctgacctgcgtccctcctcccaggatcaggaaaggctttccttgtgaagccagagtggtggccaggatcctgccccagtttctagacgacttcttcccaccccaggacatcatgaacaaagtcatcggagagtttctgtccaaccagcagccatacccccagttcatggccaccgtggtgtataaggtgaggttgcatgtgggatggggatggagtgggaaagcctggaggtggagttgcctccgacttcccagcagattcgccagcagagcccagctcctccgctttaaagcagcaatgcctctggcccccaccccacccccgccacccaggcgcagcaggtgcttcccgtccccccagccctgacactcaggcacctgcttgctccttgcaggtgtttcagactctgcacagcaccgggcagtcgtccatggtccgggactgggtcatgctgtccctctccaacttcacgcagagggccccggtcgccatggccacgtggagcctctcctgcttctttgtcagcgcgtccaccagcccgtgggtcgcggcgatgtatcctctctgggtccctggtgctggccccgtttcccttgtcaacaccgaggctcatgtttcatgataaggttttgaaacctaacctttgcaaaaaccccacagatgccagggtgacaggccctcagccccagggaagtaaaatgctgacaggggtacagaaaggagcacgtccagacatttgctgaccagggcctctcagaggggccggtgtatggcaggagggtcgcagctgaggggcctttctgtggagggcctgggtgaggggagcgagggtgggcggtggtctctgcagacgtcccgcccactcgcgggctctgtgtggctgggcttctcctgacactgcttctcattagctttggtcattgtgcctcgatcgccctctcggggaaaggcttaagtaaagatccagttcccacccccagatgctggctgccaggagtttccctttccacagcccttccccaagacagaccacaagagcctccaagcagcacagttgtcctggtgctgacagcacagccttgcccggcgtgcctggcacggctctgccctcactgcattggagcagggctagtggaggccagcggaagcaccggccaccagcgctgcacaggagccaggccaggtgagtgctgccgagtgggtgccctgcctgcagggcatccagccagccaagggttgcaggaatggaggtggaggcgctgatgcagctggaggcatccaggtggcccttccggggctctgctcgctctccaggctccctggacccctttgtagactgtttcaggagaggaactcccaggtgaggacagggaggcagcattcccctcatttgccggcctttttccttaactcctgcaccagcctcccacatgtcatcagcaggatgggcaagctggagcaggtggacgtgaaccttttctgcctggtcgccacagacttctacagacaccagatagaggaggagctcgaccgcagggccttccagtctgtgcttgaggtggttgcagccccaggaagcccatatcaccggctgctgacttgtttacgaaatgtccacaaggtcaccacctgctgagcgccatggtgggagagactgtgaggcggcagctggggccggagcctttggaagtctgcgcccttgtgccctgcctccaccgagccagcttggtccctatgggcttccgcacatgccgcgggcggccaggcaacgtgcgtgtctctgccatgtggcagaagtgctctttgtggcagtggccaggcagggagtgtctgcagtcctggtggggctgagcctgaggccttccagaaagcaggagcagctgtgctgcaccccatgtgggtgaccaggtcctttctcctgatagtcacctgctggttgttgccaggttgcagctgctcttgcatctgggccagaagtcctccctcctgcaggctggctgttggcccctctgctgtcctgcagtagaaggtgccgtgagcaggctttgggaacactggcctgggtctccctggtggggtgtgcatgccacgccccgtgtctggatgcacagatgccatggcctgtgctgggccagtggctgggggtgctagacacccggcaccattctcccttctctcttttcttctcaggatttaaaatttaattatatcagtaaagagattaattttaacgtaactctttctatgcccgtgtaaagtatgtgaatcgcaaggcctgtgctgcatgcgacagcgtccggggtggtggacagggcccccggccacgctccctctcctgtagccactggcatagccctcctgagcacccgctgacatttccgttgtacatgttcctgtttatgcattcacaaggtgactgggatgtagagaggcgttagtgggcaggtggccacagcaggactgaggacaggcccccattatcctaggggtgcgctcacctgcagcccctcctcctcgggcacagacgactgtcgttctccacccaccagtcagggacagcagcctccctgtcactcagctgagaaggccagccctccctggctgtgagcagcctccactgtgtccagagacatgggcctcccactcctgttccttgctagccctggggtggcgtctgcctaggagctggctggcaggtgttgggacctgctgctccatggatgcatgccctaagagtgtcactgagctgtgttttgtctgagcctctctcggtcaacagcaaagcttggtgtcttggcactgttagtgacagagcccagcatcccttctgcccccgttccagctgacatcttgcacggtgaccccttttagtcaggagagtgcagatctgtgctcatcggagactgccccacggccctgtcagagccgccactcctatccccaggccaggtccctggaccagcctcctgtttgcaggcccagaggagccaagtcattaaaatggaagtggattctggatggccgggctgctgctgatgtaggagctggatttgggagctctgcttgccgactggctgtgagacgaggcaggggctctgcttcctcagccctagaggcgagccaggcaaggttggcgactgtcatgtggcttggtttggtcatgcccgtcgatgttttgggtattgaatgtggtaagtggaggaaatgttggaactctgtgcaggtgctgccttgagacccccaagcttccacctgtccctctcctatgtggcagctggggagcagctgagatgtggacttgtatgctgcccacatacgtgagggggagctgaaagggagcccctcctctgagcagcctctgccaggcctgtatgaggcttttcccaccagctcccaacagaggcctcccccagccaggaccacctcgtcctcgtggcggggcagcaggagcggtagaaaggggtccgatgtttgaggaggcccttaagggaagctactgaattataacacgtaagaaaatcaccattccgtattggttgggggctcctgtttctcatcctagctttttcctggaaagcccgctagaaggtttgggaacgaggggaaagttctcagaactgttggctgctccccacccgcctcccgcctcccccgcaggttatgtcagcagctctgagacagcagtatcacaggccagatgttgttcctggctagatgtttacatttgtaagaaataacactgtgaatgtaaaacagagccattcccttggaatgcatatcgctgggctcaacatagagtttgtcttcctcttgtttacgacgtgatctaaaccagtccttagcaaggggctcagaacaccccgctctggcagtaggtgtcccccacccccaaagacctgcctgtgtgctccggagatgaatatgagctcattagtaaaaatgacttcacccacgcatatacataaagtatccatgcatgtgcatatagacacatctataattttacacacacacctctcaagacggagatgcatggcctctaagagtgcccgtgtcggttcttcctggaagttgactttccttagacccgccaggtcaagttagccgcgtgacggacatccaggcgtgggacgtggtcagggcagggctcattcattgcccactaggatcccactggcgaagatggtctccatatcagctctctgcagaagggaggaagactttatcatgttcctaaaaatctgtggcaagcacccatcgtattatccaaattttgttgcaaatgtgattaatttggttgtcaagttttgggggtgggctgtggggagattgcttttgttttcctgctggtaatatcgggaaagattttaatgaaaccagggtagaattgtttggcaatgcactgaagcgtgtttctttcccaaaatgtgcctcccttccgctgcgggcccagctgagtctatgtaggtgatgtttccagctgccaagtgctctttgttactgtccaccctcatttctgccagcgcatgtgtcctttcaaggggaaaatgtgaagctgaaccccctccagacacccagaatgtagcatctgagaaggccctgtgccctaaaggacacccctcgcccccatcttcatggagggggtcatttcagagccctcggagccaatgaacagctcctcctcttggagctgagatgagccccacgtggagctcgggacggatagtagacagcaataactcggtgtgtggccgcctggcaggtggaacttcctcccgttgcggggtggagtgaggttagttctgtgtgtctggtgggtggagtcaggcttctcttgctacctgtgagcatccttcccagcagacatcctcatcgggctttgtccctcccccgcttcctccctctgcggggaggacccgggaccacagctgctggccagggtagacttggagctgtcctccagaggggtcacgtgtaggagtgagaagaaggaagatcttgagagctgctgagggaccttggagagctcaggatggctcagacgaggacactcgcttgccgggcctgggcctcctgggaaggagggagctgctcagaatgccgcatgacaactgaaggcaacctggaaggttcaggggccgctcttcccccatgtgcctgtcacgctctggtgcagtcaaaggaacgccttcccctcagttgtttctaagagcagagtctcccgctgcaatctgggtggtaactgccagccttggaggatcgtggccaacgtggacctgcctacggagggtgggctctgacccaagtggggcctccttgtccaggtctcactgctttgcaccgtggtcagagggactgtcagctgagcttgagctcccctggagccagcagggctgtgatgggcgagtcccggagccccacccagacctgaatgcttctgagagcaaagggaaggactgacgagagatgtatatttaattttttaactgctgcaaacattgtacatccaaattaaaggaaaaaaatggaaaccatcagttgttgctgtgtgaggcttgctttgcttcatgagaacctagaccttgctgagctggagtcttaggaagcagtctcctaagtgcttctccagcaggggcagaaactgtcccaccagctaacatctggcattatggagggtcccccaggcagctgccagcagggacaggccccgtgttttctgtagccagggatgaggaagtggccccagggcatgggcctggctgggtgcttctgcaagggccttcccaaaccacagtacaggtggtcttcctgccctgcagatgggagctgtgggagctgctggagctgctggagccttcatggtcaagtgacatcataagcttatatgacatacacaagcctcaggacttggcccatggcactgaagcaggtcatcaggcccagcacagagactagagctgtgttctcacagggcccaccacccttccacctccttggccattgacacctgcgtccctggcccagctgctcccaggtaacccccaaagcagctggcacatcccacctctggtgtggccggggctgctgtgtgtccgcagggcctgccccgtctattctagcttgtttgtcctgtctgaaccagcgcctactccaagaagcctctgctcagcccagcggggatgcttctaagctccggacgagcctctcggaagccttggtgattggtggtgtagtcatcttgggatgcagatgtcttaccaacctgcaagaacaaaaaccctgtggcttcctctggtgcagggtatttagtcaatgtttgctgaggtcccgtctggttctggctaattggcaggggtcgtccacccattctttccctgctctgctgtctgtgccaggagagacgggggccagtcggccaaggggccagctcctgctgcctgctcctcttgggcacgtgcgggggccccctttctctgagcagggatagggatcagtctgccggagggatgtggtggacaggcctaaagcatttggggcggggcatgccacttgagctccctaaatctgtctcctcataggtgacaccgctccagggccccccagtggcctctcctttcagagctacctaaattctggtcacttcagagaaatggagcacccccttctccctggtccaggtgtggacagcctggcacactgagcacacctggcatggctggtaatttcagaaagaagaggggccggggtccagtgggaagcagcggtgaacccctcgtgagtgggctttgcagtccctccccatgccacggcagagctgccctcaacacagccttcctcttcctcatcggagagcacaccctgtccccttgccgagctgtgccctgtgccttcggtggtatttgattttggctgctactggctttgttgggatctggaagtcgcttcccctgcgtggtgcgtggagcactgtaagtcagatgagggaagtagccagggtgaggtgagtaccgggtggagccgccactgaagggactgggtaggggggccttgcctctacatgatgtgacacagccaaccgaggacagaggaagccccgttcctgggggtgtggggtgcacccctcagggaagcctgcagtggggcctgaggaaaggcatcctccgcgagcccacgagtctggtccatgagcaccgtgacagtgtctgtgggtagaggtggacccggccttgtgtcatcaccaggacctcttttgggaaaccatgtggacatcgcttgcgggtcccccaggctctgcagccccagcagcctggctgccttttgggcaagtggcttgagccacagaggacccagtcctgttgcagccacatcctctgggggggcccgccagtgtggccggctttctccaccctacaccaggcctccaggtgtcctggtcgggggtgtctgggccctgggtgggccctgtggacctgtgaggtcagggtcagggcatcactggaggcagagggctgaagttgtgggtctgggttccccttgtgtgcacaggcccctgccctccatgcttggtcaggcagctacccccaaaactgctaggacaggctggtcctgaggtggatcctggcccctgtaccctctggacagcccacccgcccaaccttctaccctgccccagcggcggcagtgttggccacatccttcccctcctggccccaattgctctggggaagtccaggctccggagcctgcccaggggccccccgtgatttgggcccaggactccacgtggttctctgccttcacccaagccctgaactcctcagctgccaaatccccacccatctgcacaggctgtgctcaccactgctgctcctggaaggtgcccctcagtgggacgcccacctcctctctgggcttctgtgtttgggagccctgctgcccccacccttggtcagtccccatgtcctgctggcctgtcaggcagggcagaaaatccacccagaaatgctgagcaggatgagagtctagttgggcccagcctcattatttagaagggatggaggcctagggagcatgcttctagcctgagcccagcagggccccgcccatgtcccaggtctgcaccagggacagctcctgccgaggcctgacctgccccttctccctcaggtgctgctggttgaccagcctctggccctaggagaccccgtagcgactgagggtcccagcaggccatgcagctttgccaaggtacgagcccctccccagcaggggacagatgtggggaccctcccaggcaggagcagctgggtgcctggtgctgccatctgctgcctgcctggttcttgtcctcacattggaggtcagtgtgagggctctgcctcgggaaaggccatggagcttgccctgtccagggcctcccatgtgcactgagcctgggaagagagggttggagttgagccttttaccctgggaatgctgcctggaggatggtgcgggtgtggggtggcaccctgccaggcagggccctgcctccctgcgcccactggaactcgggcaggcaggggtgtaggtgcctcctctagagccgtccggtgggggcccccggcagtggtggtggtgtccactggccagcagctgccccttcagccaggacagtaggcctgacgctgtccccagcagctccaaggtggatttgtggaagggggtagagggcacgtagaggccccatgacctccccagggttctgggagggctgtgcccccttagccagcaccatgctgggtgatatagtcagatcctgttacccctgttgtggaggtgaggaaacaggttagtggggaggacatgactaaggtccatgctgagtcgctagagctgcacccagaaccactgctgggaccccatgcctttctgcttaccccttgtgccgggagatgccaagagatgctgggagccagccccacctctgcccttggagtcatggctacggaaagggcattcggaccggtccctgacctcaccggggagggccgaaccctgttcctgaggagccagggcttcctagaggaggtaggccttctagtcactccttcatctgcaggcactccacagagctctctgtgccagcccccagcacggagggctgaccttagtcgagtggagatgccccagtgccaggcagtagggatgatgtctcctgaggcccagatggaagggactggactagtctcatggggctgatggtggggccaggccttgaccagggacccagtgtagggggtgcagagacccctctgagttcctcacacatccctggggccctccccatacacttcctatcctgactgcgggcaagagggagccccagttcgccttccctatgctgggcacccacagtggggctgggcacccccgccatgcccctgccctgtccttcccctgagagcctcggtcccacctccaaggtgcctcagaggacagcaggggcagcgggcagaggccgagatgcctcctcattccaggctcagctgcccttcttggggcagcccacacctgagagtctcctgcagttggtcaggcctgaggagggcaggggggtgcctgctgtccctctgctgaccacagtggcatttagcctgggcaccgcgcccagcacagtccatgctgcacaggtgccgtgggctccacagagccctgcctgacatgcatgtgttacgtttcgggtgccgatgcccttgggcggcacttctccgggcagaacccccaggccaccgctccggttccggttccgctgcatctggggctctcggcaggctgtggtcctccggccagcctgggggcatctcagtccctcagccccacaggggcctgccccgcagcctgggcctcgagccccgtctccgcacgctgtgccgaatctggctgcccatcagctccctgcgtacccagactgtgccctgccatgcccgtggctcttcccaggagtgccctgtggcctccccctggcttgctgggctgattccctcctgtgtctcaaacagagctcacctttgccatcactgctgtcctcaccggccggtgccagaggcccgtgtctgtgtaccctgtgtctgcacctctgggcagggcctggctctgaccaacccgggcttccagtgtccacagacctaaggcccagggcgcctgggggctggagcaagagaagcaaaaggagccaagggtgggggtttggggttcttgtgagggcccagccccaggaccccaggaccaggacacccaggagccccagggcccagccccagttcagaaggcaggggccttctgagggagcttaagggtcccacagcccaggacccccaccagggccagtggccagcgttgggggactcagcctcctcgtcgctcgtcctctctgtttctcccaccttttgccccctttctccttgcctgttcccacccgaggccccctcttggcctgcgtgagccggggcggcactgaactgggggccgatccgcctgggcggcggtgagaggcagggccgggagccgggccgctgggtttgggcctggcccgctcgccgcaatattgatggcccgtcagtgcagccctgattcctgtgctttcagttaaaaggtttctgttgttgtagcttatgcagttgctctgttgctatggaaacgtgacatcaaaatgacgtttcccgtttaaaagcttttaactaaattcctgcctgtcagatgtaggccccattttgagcgtggagctgccttcgagcgagcgtgagcggcgcctcccgcccatggtgcgtggggccgggccggggccctcgctgagcgcgctctctcaccccacaggcgcctccggcatggcggcggccgaggggcccggctacctcgtgtctccccaggcggagaagcaccggcgggcccgcaactggacggacgccgagatgcgcggcctcatgctggtctgggaggagttcttcgacgagctcaagcagaccaagcgcaacgccaaggtgtacgagaagatggccagcaagctcttcgagatgaccggcgagcgcaggctgggcgaggagatcaagatcaagatcaccaacatgaccttccagtacaggtgggcgagcgggcagtgtgggccccaccaggacgggcgggcccgggcgtggcgggccgctcctgactttcttggagctctgagtcgggacgatgtgtgggtcgtggcctgcctgtcggtctcctctggccgggtatgggcagaaccccacggggtgagacggggcccacggaaaccgtgtgtgcagccttccattggggaagtggggaaactgaggcccagcaagggcaggaaaccagtctaagagctgaggggtagcaggggtggggctggtgctgggcagaggccaggatggctcccaggacgtatgggcggtctgggcactgtccctcggaggcagcaacactcatggtggtgcccactgacctcacaccctgctcccccatagggaggcggcggctgccagtgccctccccaccaccaagctcccaagctcagcaggggtttcaggggcctactgcgtcattggggaaattgagactgcaagtgagaaggaggctcagtgctctgcgacttggagcatccactgagcctctgccatgagccggtgagccccactggggctggccctagggtcacggtggggtatttccagaaatcaccaggtgaggtgcaggaccagccagcgcatgggtggggcttacggtgcgaagaagaaagaggtggaggcctgccctggcccaggactcccagcgtgggggctcccggcctggccccacctctgctcctgctacatggcaggtgggcccttcctgccctggcaacctgcagggaaggccggaggggaccacccagccagggagatgttggcgtctaggaggggacaggtgtggtcccacacacccagcatcttaaagtgcgtgggtccccagcccattaggacagggtcccgggtgggcaggggtcatggtggggtgaaggtctcaggcacaggcaaggtcacaggtgcggtgagggtcttgcagggtgtgaaggtcataggtgtgcggtgaaggtcacaggtgtggggtgatggttttgggtgtggggagggtcttgcacggagcgagggtggcagcaagagctggaagctgcagggggagaatggcagcagagagcacccggccctgtgggcggcctggacagggctgggcctggggctgccggagagcctgtcagcttccaggatgggagtggcctcactcagctgctccacctccgggtcaggcaggtgagcctggggcagagaggctgagagcacctgagccacttgtgggagaggccacccccactgcccccctcaggcgaggagccggcctccagcacagcagaagggaacccccagtccccagccctagtgggagtggggaagaggcccagcaaggccccggacagaccgccagcctgtgaggtctccgctttcagttgcgttgatttgattttttctgagccttgaaggaggggtccggggcctggccctgcccaaaggcccctaggcaggccccaaagccgggacctagggtgctgagcatgacggatgttgggtttgagcggctggcttgcgacgtgagggctgaggtgtgagcctgggtatcttcagaggttcggtggacacaggcagctgcccgcggccccactgttcccgtggcctcctagtcctgctcaggcacctggtgaggaagggacgcagagggcagtgggaggtggccacgactgttccagcaggctcccctctgactcaggaattcacgggcaccacctccctggctggctctggttggtgtctggccaggttattcattatttatgctgaaagcctcttcagagtcccaggggagggtttctgtctccattcctggaggctgagagatgagggtgcagcagagtgggggcctccactccagaccctgcagtctgggctggccaagggctgcaccggtgcactgcacgtcatggctgatgaagcacttccacaccgcagcccctcagagctgccacagtcagccttagttcaccgagggggaagctgaggcccagagcatgagagggacttgcccagggccacatagtccttagcagaggaagctgtggctgggtgactcgatctttgtcctttttctttatacccgcagtctccccatagcagaggcttttcttttttttttctttttcttttttttttttttacaagaactctttatatattaaggctgttgggctgaagaagcctgagagggtggctggttctgtggagcatggtttgttgaagtacagtttgggggcctcctacactgagaataggccttttctcgtttctccaaagagtgggctggctcaagtagggcagagagagaagcctggggcagaggttagggatgggcacccagcgcctgccctcacacgctctgtgctggtgtcttcacagccacgtgccaccctgggcagcatcccctgctcaccatctggctgtgcctgtttgctgggggcacctcattcagaatccagcttattgtttccaacggccaatggccacaccctggcaggtagcaagagtaggagagaggagacacccactccgagcacaggttgggtttggagcccggccttggggcactctgtcactcaaaggcagagtggggagtgggcactgggccttaggaggtactgggtccagtgaggcagagatgcccctgccccacccccaccttgtggcttcttccctggcctggccagagctgtctggccgccatggggccctgtgtctcctgccttgacctcccagagggcagccgaggcccaggggaggcctggggacttagcctctcagggcaggacctgtctgcaggagtaggtgggtgctgggggtcccagtggtaatgaggcatcaggcagtgtgggaaggggcccatccggcccaccccagggcctctgggcaggttgcaggttgtagcgctggatctaggctcctgcccagactgtaggttcaaccaagaatggcatgggagcccagcctgctgtttgctttattaaatctgccctgtagctgggggaggggcttactttgatcatcactatgtcattgatataaaaatagaggctcagagaggtgaatgaacctgcccaaagtcacacagcaaagtgtggagatgagatactgactcagggctgtggacactgaagcctgtgctctaacgccagtggctgtcgctccctgaggcattctctcccgaacaacacagttattatattacaaaatattatcactatatttatatatcttataataccttattattacaataaaaccttattactctacctttcaaaatgaattatttaaaaagcagtatttgctcattgcagagagtctagaaactatagaaaagcaagggaaaagcaataggaccagccccaaggtcccagcatgcacagataaccttagtaatactgggacgtgtgcttcctttttaacatctgagcccgtgtaggtcctgaagcccagcttctttctaagtccattgtcatcttgaccctggagcctggccgattttgctggggaggcccttgccagccgagagcggctcctgcctgtgccggcgtggcgcgcccctctgctgaggctgggcaggacaggggctgggccagctctgtttctcacccttggctcttgtgtctctcgtttcaggaaattaaaatgcatgacagatagcgagtccgccccgcccgactggccctattacctagccattgatgggattctggccaaggtccccgagtcctgtgatggcaaactgccggacagccagccgccggggccctccacgtcccagaccgaggcgtccctgtcgccgcccgctaagtccacccctctgtacttcccgtataaccagtgctcctacgaaggccgcttcgaggatgatcgctccgacagctcctccagcttactgtcccttaagttcaggtagtgtgtctgcttgtccttcccctgccctggggtatctcagcccccaccatttagagaaagggactgggagtggcaaggccggcggcggcggccacagtggttgcagaggccgtggctgcgggcagcgcctccagggacaggcggcctcagaccagggagggctttagtgtccacaggcagaccgagtttgtctcccagctccatcacttttgagctgcacggaaagttccttgacttctctggcctcagtctccctcctataaaatgggggtaaatcagtacctttctcagagggtggctgggagcatcacaggagagaagacgcagcatggggcccggcacacggagggagaccaagccccagaccccagaatgcgccccctggcctcccttagcccacacagaccccaccctcacaggctagctgccctctcagcactggggagggtgtcgggctgcacctcatcacgtgttgccgtgggcatgacccgtcccctctgccatccatcccacacctcagacccgtcccgtgctggccacgtgactgtgcctgcaagatgctcacagggcagccgggagccaggcagcatgcaggacagacacctgcggggtgggcctggggagcccagagaaggtgcttttgaggaggggacatttggggtgggctttcaaggtaaaatagaagttggccatttggaggcaagaacaggaagattgtggatttgagtcacagcttctcccctgccctggtcttcaagtctttctgacaggaggtgtcagaaaagtatctttagtagagaaggcgtctccgaggagggtccctctcatgccgggggccgctgcttgactcaggatttctcattgaagacctgagacaaaaacgcttttgctggcagctagaaggaaccagcaggaggcctgagatttgtggctgttgttcccgtggactgagcccagttctcagactcagctgcctggggccttgcacaggactggggcgtgggggctgccctccctgatcaggcccaaagcgcggatctcacgcccctgaggttggctgtaccctctcagctcagagcagagtgtgggccagggatgagcaggcactggagcagggccctggggtctgtgggttttggcagctccctgcccttcagggaggtctgctgagaccacgggtggcccctaccccagcagcagagctctcaggaggcgcccacagggctggactgcctttactcaccacctctaccagagctctgaggtcctggggagagagcccaggcctcttgtgggccccacaccctctaggtgcctgtccttctgcctctctaccaaggtgtgccggccccatttctaggccgccgggagataagggggctcacatctcaggcccttccttctgggacctcagtttccccatctgcctaaggccgggtggggctggtggtcttggcttccctacaggggtcctgagtactctgcactacccagcaccccccacccctgccttcatctctccctgggggtggtctctccacccctggcccccaactggggctgagcccccacctgcccagtttggtgggtgaagggtgctccctggcaggatatgcccctctgcagcccagaacatcccaccctttccagaccgaaggggtgtggattgtcctgggaccctggtcattggggtcatccgctagtcgcaaaggacggcaatgcctgtggcctctctttctttctttttctttttttttttttttgagacggagtctcgctcttgtgcagagagcagtggcgcgatcttggctcactgcaacctccgcctcgtgggttcaagcgattctcctgcctcagcctcccgagtagctgggattacaggcacccgccacaacgcctggctaatttttgtatttttagtagagatggggtttcaccatgttggccaggctggtcttgaactcctgacctcaggtgatccacctgcctctgcctcccaaagtgctgggattacaggcataagcctccacacccggccacccctgttactttctgtcaaaggcggtgggttctggcccctcctttgcacatggaatatgagaccctgagtaagtgacctgactccctggggcctcagtttccccatttgcccagtaggattgtcgggagggtccggtgaggcccctggtgtgcccaggctctgtggccagcacgtccacagccggcactgtccttccaggtcggaggagcggccggtgaagaagcgcaaggtgcagagctgccacctgcagaagaagcagctgcggctgctggaggccatggtggaggagcagcgccggctgagccgcgccgtggaggagacctgccgcgaggtgcgccgcgtgctggaccagcagcacatcctgcaggtgcagagcctgcagctgcaggagcgcatgatgagtctgctggagaggatcatcaccaagtccagcgtctaggccagcaggcggcggcggcggcggggccgggcggctggtggtactgctcaggccacccagggcaggccactcaggccaggcgggcaagggggccgccccgcgagcggagaccgccttccacctggcctctggcaggatgtcccttctgaggggtattttgaggaacccccaggccctggggaccgtgaggctccagtctccagcatgaatgcccttcctcggacacaggccagggcctctggggttcactccgagtaagaacgtcctagagccactctccagtgtcgttactatcaatgatacttgacgtggctttgatattaaacgtatactttttcattcttgcctggaacgcacagtttgctgttgctggcttggtgaggatgccctgattgatggatcccgaaaatgaaagcagatggaaacgggttggggcaggctggagctgggggagctctctcctgaagggaaccctgtgtcctccctcaccaggacctctgcgtctctccttaaatggcctctgacgcctgatgaaaaccccagcgaccttccaggaggcttttattcagctctgtttggagcatcaggtgtttccactgcctccttagcaatgacactaataaaagtcgtaacacctgttcacatgcacagccctgttgagtgttctgggtgctggagatatcatggtggatgacacaaaggccctggcctcttggagcttatgctcccatgcggggaagacacatgggtcagtagagaaatggttgcaggttgtgataagtgctggaagggaggggttggcctgaggacacggaggcagacatacgtggagctgggaacagtggccacacagggaacggccagtgcgaaggcccagaggcagaggacactggagcaagcccaggagcagctaggaggctggtggccagcagccaggccacggaagcccgtgcagcccgtggggaggagtgttcatgcttttcaagcttagtgggagtcttttggccagtgcagctctgggtctgacatcggtgggggacagaggggtggtggagcggccacagctgcaagctcacctcactgccggcccttccaccagtttcaaactctttctagaagctccagctttcccaaagctgaattctctatgagcctccttggccgggactcgggcgtctggttgccctggctgcaaaggaggctggggccaggtgtgtttgagtcacctcctggaattaggcaagttgctgcccaaatagaaggttgttggcaggtgggtcagcaggtgaacagcatggtttgactcagggttcagaaaaatctccctctggctgccaagcgagcaggccgtggagacaggtgcagaggcaggtgtggcagcaggcatcctgccaggcagtgctgcagtcatcctgcgacaagcagcagcagctcatcctaccctctagggggtcttgaggtcagccaggcaagagagcagcttggactccactgggtgtgggaccagcctgtggaccatggtggtgtggagggtgccctcggcctgcctgtgtgaaggagaggccggcgtgttctgtggagcccaaaggggagctgggcaagcaggattcacttcactctgagggtcctggagctcccaccctcctcagccatctccccagagcctgtgtgccgaggactcggcccatgttgctgtgggatgagaggcagagtgtcgtgagggtgtaaggagcggcggcagtggtgggaggagggagcagcagccagcgctacggtgccagtttccagctgccagatgacgccgctgaccctgtggttgagaagagatgcacagagccagctcttgcaagccagtgtggctgccatagcacctgccgagaagcagaaggaagggtggccccaggaggacagaggatgcgggcacatctgatgcgggcctgagttttgggagcttttgctctagccagtttccagctccgggacccacccgcctcgtaggcaagacaccacccaagaaatcatttgcttaacaaacacactgggctccaactggacacctgtgccaccctagatgctgggaacccagccatgacacaggcacctgcccccagctgctgaccactgaggctggctagcagctcccatggggccagtgtggggttccccagcctcctaacagggagccagtcacaagccctcgagagggaagggtgcccgcggccctggcaggaaggttaggctggacgctcccacaagacataacagatggaggttctaaatgatgtagcaacttcttcaccctgaaactgctgtagagtcagccatgacgcaccggtacttcagtaactgccaggcatccgggacagcacaccgcgagtcgctgctgtgcttgggttagaagtggtttggtctgttttcttctcgccctctctaatcagagtcagtgattcatgcccttccatcaccttagagaaggggcaggcgctgcccgaccttctccaggctggagcagcatcgcctcatgtcagcagaactcagctgtagaatatcgtggggttggtgcctttcatcagcagcatgtccttaacaactttctgatttcttccttagttgttggtccattaaggagaaaaaaaatgatctcagccattgctaaaatatttgataagattcagcaaagcagcatgttaacattgaaaactagaatcaggagccaggcagatgtgcttgcttttcacctgtagtatttcatgttgttttgacgtttttagctaatgcattaagataaataaacaaaagccgggcacggtggttcacgcctgtaatcccagcactttgggaggctgaggcgggaggatcctctgaggtcaggagttcaagaccagcctgaccaacatggagaaacctcgtcattactaaaaatacaaaattagctgggcgtggtggtgcatgcctgtaatcccagctacttgggaggctgaggcaggagaatcgcttgaacccgggaggcggaggttgcagtgagctgagattgcaccactgcactccagcctgggtgacagtgaaactcggtctcaaaaaaaaaaaaaaattaaaaaaagataaataaaataagcaggataagaaatgaagaaagtagagttacctttgttttcagatttcatttttgtatacccagaaagccaaatgtacaaaagactgggagctctttaaaccagcttaaacttgttgaaaatgaggatgaagaaatatcccattcagagttggaatgaatttaacccagaaggaacaggacctctactgaagagaactatgcagtcttactgaaaaatctaaataatacctgagcgctggagaaacttcgcacactcctgaaagctccaaagtcaatgtcatcattttattaatgtcattccaaacatagtctcaataatatcacttcttggttttgacatggacgcgatgatgtttaaattcatatgaaaaaagaacggggccaaaagtccaaggccagtcagcgtgagaagaccgctcggcctccctcggagtcggggagttggaaccgcagactgagatcatgtggctgctggaggccaggacgaacgtcgggaaatggagactcctgcgttgctggtgggatgtggtgcagccgcttccaggagcaatttggtgtcccgtcctaaagctgaagaaacgcatttcctctggtcagtgccactcctagacaggccaccctgcggcagccgtcctcaaactggtctgaggacccctcaacgctcttaaaaatcattaaaagtgggccaggtgcggtggctcacacctgtaatcccagcactttgggaggccaagacaggcggatcacgaggtcaggacattgagatcatcctggctaacacggtgaaaccccgtctctactaaaaatacaaaaaattagccgggcgtggtggcgggcgcctgtagtcccagctacttgggaggctgagccaggagaatggcgtgaacccaggaggtggagcttgcagtgagctgagatcactccactgcactccagcctgggcagcagagcgagactctgtctcaaaaaaaaataataaataaataaataaaaataaaataaaataaaattcattaaaagtgccaaagaacttttgcttatgtgagttctaatgaccaatattaatacacattagaatatcttattagaaattaaacctgagacctttagaaaacatgtattcatttcaaaatagcaataaacccatgacatattaacataaataacaattgtatgaaaaatatattttccaaaacaaaaagttttcgggagaagtgtggcatagttttacatggtcgtaaatctctggcttaagagaagcccactggcctctcagcaggctctgggtccgtccactttgggggtgttttggttgtgaagtataggagtgaatggagaagctcattcttacccagatgtgtatttgaaaagaaaaggaacattttaataacctttgcaaataatcggtatattcttccgtgatcctattccaacactggacaggtggtggtttgttttttttttttggagacggagtcccgctctgtcactcaggctggagtgcagtggcgcgatttcagctcactgcaagctccgcctcc

SEQ ID NO: 2 - RNA, 13481 nt, Homo sapiens
  source 1..13481: mol_type = mRNA
gctgccgggacgggtccaagatggacggccgctcaggttctgcttttacctgcggcccagagccccattcattgccccggtgctgagcggcgccgcgagtcggcccgaggcctccggggactgccgtgccgggcgggagaccgccatggcgaccctggaaaagctgatgaaggccttcgagtccctcaagtccttccagcagcagcagcagcagcagcagcagcagcagcagcagcagcagcagcagcagcagcagcagcaacagccgccaccgccgccgccgccgccgccgcctcctcagcttcctcagccgccgccgcaggcacagccgctgctgcctcagccgcagccgcccccgccgccgcccccgccgccacccggcccggctgtggctgaggagccgctgcaccgaccaaagaaagaactttcagctaccaagaaagaccgtgtgaatcattgtctgacaatatgtgaaaacatagtggcacagtctgtcagaaattctccagaatttcagaaacttctgggcatcgctatggaactttttctgctgtgcagtgatgacgcagagtcagatgtcaggatggtggctgacgaatgcctcaacaaagttatcaaagctttgatggattctaatcttccaaggttacagctcgagctctataaggaaattaaaaagaatggtgcccctcggagtttgcgtgctgccctgtggaggtttgctgagctggctcacctggttcggcctcagaaatgcaggccttacctggtgaaccttctgccgtgcctgactcgaacaagcaagagacccgaagaatcagtccaggagaccttggctgcagctgttcccaaaattatggcttcttttggcaattttgcaaatgacaatgaaattaaggttttgttaaaggccttcatagcgaacctgaagtcaagctcccccaccattcggcggacagcggctggatcagcagtgagcatctgccagcactcaagaaggacacaatatttctatagttggctactaaatgtgctcttaggcttactcgttcctgtcgaggatgaacactccactctgctgattcttggcgtgctgctcaccctgaggtatttggtgcccttgctgcagcagcaggtcaaggacacaagcctgaaaggcagcttcggagtgacaaggaaagaaatggaagtctctccttctgcagagcagcttgtccaggtttatgaactgacgttacatcatacacagcaccaagaccacaatgttgtgaccggagccctggagctgttgcagcagctcttcagaacgcctccacccgagcttctgcaaaccctgaccgcagtcgggggcattgggcagctcaccgctgctaaggaggagtctggtggccgaagccgtagtgggagtattgtggaacttatagctggagggggttcctcatgcagccctgtcctttcaagaaaacaaaaaggcaaagtgctcttaggagaagaagaagccttggaggatgactctgaatcgagatcggatgtcagcagctctgccttaacagcctcagtgaaggatgagatcagtggagagctggctgcttcttcaggggtttccactccagggtcagcaggtcatgacatcatcacagaacagccacggtcacagcacacactgcaggcggactcagtggatctggccagctgtgacttgacaagctctgccactgatggggatgaggaggatatcttgagccacagctccagccaggtcagcgccgtcccatctgaccctgccatggacctgaatgatgggacccaggcctcgtcgcccatcagcgacagctcccagaccaccaccgaagggcctgattcagctgttaccccttcagacagttctgaaattgtgttagacggtaccgacaaccagtatttgggcctgcagattggacagccccaggatgaagatgaggaagccacaggtattcttcctgatgaagcctcggaggccttcaggaactcttccatggcccttcaacaggcacatttattgaaaaacatgagtcactgcaggcagccttctgacagcagtgttgataaatttgtgttgagagatgaagctactgaaccgggtgatcaagaaaacaagccttgccgcatcaaaggtgacattggacagtccactgatgatgactctgcacctcttgtccattgtgtccgccttttatctgcttcgtttttgctaacagggggaaaaaatgtgctggttccggacagggatgtgagggtcagcgtgaaggccctggccctcagctgtgtgggagcagctgtggccctccacccggaatctttcttcagcaaactctataaagttcctcttgacaccacggaataccctgaggaacagtatgtctcagacatcttgaactacatcgatcatggagacccacaggttcgaggagccactgccattctctgtgggaccctcatctgctccatcctcagcaggtcccgcttccacgtgggagattggatgggcaccattagaaccctcacaggaaatacattttctttggcggattgcattcctttgctgcggaaaacactgaaggatgagtcttctgttacttgcaagttagcttgtacagctgtgaggaactgtgtcatgagtctctgcagcagcagctacagtgagttaggactgcagctgatcatcgatgtgctgactctgaggaacagttcctattggctggtgaggacagagcttctggaaacccttgcagagattgacttcaggctggtgagctttttggaggcaaaagcagaaaacttacacagaggggctcatcattatacagggcttttaaaactgcaagaacgagtgctcaataatgttgtcatccatttgcttggagatgaagaccccagggtgcgacatgttgccgcagcatcactaattaggcttgtcccaaagctgttttataaatgtgaccaaggacaagctgatccagtagtggccgtggcaagagatcaaagcagtgtttacctgaaacttctcatgcatgagacgcagcctccatctcatttctccgtcagcacaataaccagaatatatagaggctataacctactaccaagcataacagacgtcactatggaaaataacctttcaagagttattgcagcagtttctcatgaactaatcacatcaaccaccagagcactcacatttggatgctgtgaagctttgtgtcttctttccactgccttcccagtttgcatttggagtttaggttggcactgtggagtgcctccactgagtgcctcagatgagtctaggaagagctgtaccgttgggatggccacaatgattctgaccctgctctcgtcagcttggttcccattggatctctcagcccatcaagatgctttgattttggccggaaacttgcttgcagccagtgctcccaaatctctgagaagttcatgggcctctgaagaagaagccaacccagcagccaccaagcaagaggaggtctggccagccctgggggaccgggccctggtgcccatggtggagcagctcttctctcacctgctgaaggtgattaacatttgtgcccacgtcctggatgacgtggctcctggacccgcaataaaggcagccttgccttctctaacaaaccccccttctctaagtcccatccgacgaaaggggaaggagaaagaaccaggagaacaagcatctgtaccgttgagtcccaagaaaggcagtgaggccagtgcagcttctagacaatctgatacctcaggtcctgttacaacaagtaaatcctcatcactggggagtttctatcatcttccttcatacctcaaactgcatgatgtcctgaaagctacacacgctaactacaaggtcacgctggatcttcagaacagcacggaaaagtttggagggtttctccgctcagccttggatgttctttctcagatactagagctggccacactgcaggacattgggaagtgtgttgaagagatcctaggatacctgaaatcctgctttagtcgagaaccaatgatggcaactgtttgtgttcaacaattgttgaagactctctttggcacaaacttggcctcccagtttgatggcttatcttccaaccccagcaagtcacaaggccgagcacagcgccttggctcctccagtgtgaggccaggcttgtaccactactgcttcatggccccgtacacccacttcacccaggccctcgctgacgccagcctgaggaacatggtgcaggcggagcaggagaacgacacctcgggatggtttgatgtcctccagaaagtgtctacccagttgaagacaaacctcacgagtgtcacaaagaaccgtgcagataagaatgctattcataatcacattcgtttgtttgaacctcttgttataaaagctttaaaacagtacacgactacaacatgtgtgcagttacagaagcaggttttagatttgctggcgcagctggttcagttacgggttaattactgtcttctggattcagatcaggtgtttattggctttgtattgaaacagtttgaatacattgaagtgggccagttcagggaatcagaggcaatcattccaaacatctttttcttcttggtattactatcttatgaacgctatcattcaaaacagatcattggaattcctaaaatcattcagctctgtgatggcatcatggccagtggaaggaaggctgtgacacatgccataccggctctgcagcccatagtccacgacctctttgtattaagaggaacaaataaagctgatgcaggaaaagagcttgaaacccaaaaagaggtggtggtgtcaatgttactgagactcatccagtaccatcaggtgttggagatgttcattcttgtcctgcagcagtgccacaaggagaatgaagacaagtggaagcgactgtctcgacagatagctgacatcatcctcccaatgttagccaaacagcagatgcacattgactctcatgaagcccttggagtgttaaatacattatttgagattttggccccttcctccctccgtccggtagacatgcttttacggagtatgttcgtcactccaaacacaatggcgtccgtgagcactgttcaactgtggatatcgggaattctggccattttgagggttctgatttcccagtcaactgaagatattgttctttctcgtattcaggagctctccttctctccgtatttaatctcctgtacagtaattaataggttaagagatggggacagtacttcaacgctagaagaacacagtgaagggaaacaaataaagaatttgccagaagaaacattttcaaggtttctattacaactggttggtattcttttagaagacattgttacaaaacagctgaaggtggaaatgagtgagcagcaacatactttctattgccaggaactaggcacactgctaatgtgtctgatccacatcttcaagtctggaatgttccggagaatcacagcagctgccactaggctgttccgcagtgatggctgtggcggcagtttctacaccctggacagcttgaacttgcgggctcgttccatgatcaccacccacccggccctggtgctgctctggtgtcagatactgctgcttgtcaaccacaccgactaccgctggtgggcagaagtgcagcagaccccgaaaagacacagtctgtccagcacaaagttacttagtccccagatgtctggagaagaggaggattctgacttggcagccaaacttggaatgtgcaatagagaaatagtacgaagaggggctctcattctcttctgtgattatgtctgtcagaacctccatgactccgagcacttaacgtggctcattgtaaatcacattcaagatctgatcagcctttcccacgagcctccagtacaggacttcatcagtgccgttcatcggaactctgctgccagcggcctgttcatccaggcaattcagtctcgttgtgaaaacctttcaactccaaccatgctgaagaaaactcttcagtgcttggaggggatccatctcagccagtcgggagctgtgctcacgctgtatgtggacaggcttctgtgcacccctttccgtgtgctggctcgcatggtcgacatccttgcttgtcgccgggtagaaatgcttctggctgcaaatttacagagcagcatggcccagttgccaatggaagaactcaacagaatccaggaataccttcagagcagcgggctcgctcagagacaccaaaggctctattccctgctggacaggtttcgtctctccaccatgcaagactcacttagtccctctcctccagtctcttcccacccgctggacggggatgggcacgtgtcactggaaacagtgagtccggacaaagactggtacgttcatcttgtcaaatcccagtgttggaccaggtcagattctgcactgctggaaggtgcagagctggtgaatcggattcctgctgaagatatgaatgccttcatgatgaactcggagttcaacctaagcctgctagctccatgcttaagcctagggatgagtgaaatttctggtggccagaagagtgccctttttgaagcagcccgtgaggtgactctggcccgtgtgagcggcaccgtgcagcagctccctgctgtccatcatgtcttccagcccgagctgcctgcagagccggcggcctactggagcaagttgaatgatctgtttggggatgctgcactgtatcagtccctgcccactctggcccgggccctggcacagtacctggtggtggtctccaaactgcccagtcatttgcaccttcctcctgagaaagagaaggacattgtgaaattcgtggtggcaacccttgaggccctgtcctggcatttgatccatgagcagatcccgctgagtctggatctccaggcagggctggactgctgctgcctggccctgcagctgcctggcctctggagcgtggtctcctccacagagtttgtgacccacgcctgctccctcatctactgtgtgcacttcatcctggaggccgttgcagtgcagcctggagagcagcttcttagtccagaaagaaggacaaataccccaaaagccatcagcgaggaggaggaggaagtagatccaaacacacagaatcctaagtatatcactgcagcctgtgagatggtggcagaaatggtggagtctctgcagtcggtgttggccttgggtcataaaaggaatagcggcgtgccggcgtttctcacgccattgctaaggaacatcatcatcagcctggcccgcctgccccttgtcaacagctacacacgtgtgcccccactggtgtggaagcttggatggtcacccaaaccgggaggggattttggcacagcattccctgagatccccgtggagttcctccaggaaaaggaagtctttaaggagttcatctaccgcatcaacacactaggctggaccagtcgtactcagtttgaagaaacttgggccaccctccttggtgtcctggtgacgcagcccctcgtgatggagcaggaggagagcccaccagaagaagacacagagaggacccagatcaacgtcctggccgtgcaggccatcacctcactggtgctcagtgcaatgactgtgcctgtggccggcaacccagctgtaagctgcttggagcagcagccccggaacaagcctctgaaagctctcgacaccaggtttgggaggaagctgagcattatcagagggattgtggagcaagagattcaagcaatggtttcaaagagagagaatattgccacccatcatttatatcaggcatgggatcctgtcccttctctgtctccggctactacaggtgccctcatcagccacgagaagctgctgctacagatcaaccccgagcgggagctggggagcatgagctacaaactcggccaggtgtccatacactccgtgtggctggggaacagcatcacacccctgagggaggaggaatgggacgaggaagaggaggaggaggccgacgcccctgcaccttcgtcaccacccacgtctccagtcaactccaggaaacaccgggctggagttgacatccactcctgttcgcagtttttgcttgagttgtacagccgctggatcctgccgtccagctcagccaggaggaccccggccatcctgatcagtgaggtggtcagatcccttctagtggtctcagacttgttcaccgagcgcaaccagtttgagctgatgtatgtgacgctgacagaactgcgaagggtgcacccttcagaagacgagatcctcgctcagtacctggtgcctgccacctgcaaggcagctgccgtccttgggatggacaaggccgtggcggagcctgtcagccgcctgctggagagcacgctcaggagcagccacctgcccagcagggttggagccctgcacggcgtcctctatgtgctggagtgcgacctgctggacgacactgccaagcagctcatcccggtcatcagcgactatctcctctccaacctgaaagggatcgcccactgcgtgaacattcacagccagcagcacgtactggtcatgtgtgccactgcgttttacctcattgagaactatcctctggacgtagggccggaattttcagcatcaataatacagatgtgtggggtgatgctgtctggaagtgaggagtccaccccctccatcatttaccactgtgccctcagaggcctggagcgcctcctgctctctgagcagctctcccgcctggatgcagaatcgctggtcaagctgagtgtggacagagtgaacgtgcacagcccgcaccgggccatggcggctctgggcctgatgctcacctgcatgtacacaggaaaggagaaagtcagtccgggtagaacttcagaccctaatcctgcagcccccgacagcgagtcagtgattgttgctatggagcgggtatctgttctttttgataggatcaggaaaggctttccttgtgaagccagagtggtggccaggatcctgccccagtttctagacgacttcttcccaccccaggacatcatgaacaaagtcatcggagagtttctgtccaaccagcagccatacccccagttcatggccaccgtggtgtataaggtgtttcagactctgcacagcaccgggcagtcgtccatggtccgggactgggtcatgctgtccctctccaacttcacgcagagggccccggtcgccatggccacgtggagcctctcctgcttctttgtcagcgcgtccaccagcccgtgggtcgcggcgatcctcccacatgtcatcagcaggatgggcaagctggagcaggtggacgtgaaccttttctgcctggtcgccacagacttctacagacaccagatagaggaggagctcgaccgcagggccttccagtctgtgcttgaggtggttgcagccccaggaagcccatatcaccggctgctgacttgtttacgaaatgtccacaaggtcaccacctgctgagcgccatggtgggagagactgtgaggcggcagctggggccggagcctttggaagtctgcgcccttgtgccctgcctccaccgagccagcttggtccctatgggcttccgcacatgccgcgggcggccaggcaacgtgcgtgtctctgccatgtggcagaagtgctctttgtggcagtggccaggcagggagtgtctgcagtcctggtggggctgagcctgaggccttccagaaagcaggagcagctgtgctgcaccccatgtgggtgaccaggtcctttctcctgatagtcacctgctggttgttgccaggttgcagctgctcttgcatctgggccagaagtcctccctcctgcaggctggctgttggcccctctgctgtcctgcagtagaaggtgccgtgagcaggctttgggaacactggcctgggtctccctggtggggtgtgcatgccacgccccgtgtctggatgcacagatgccatggcctgtgctgggccagtggctgggggtgctagacacccggcaccattctcccttctctcttttcttctcaggatttaaaatttaattatatcagtaaagagattaattttaacgtaactctttctatgcccgtgtaaagtatgtgaatcgcaaggcctgtgctgcatgcgacagcgtccggggtggtggacagggcccccggccacgctccctctcctgtagccactggcatagccctcctgagcacccgctgacatttccgttgtacatgttcctgtttatgcattcacaaggtgactgggatgtagagaggcgttagtgggcaggtggccacagcaggactgaggacaggcccccattatcctaggggtgcgctcacctgcagcccctcctcctcgggcacagacgactgtcgttctccacccaccagtcagggacagcagcctccctgtcactcagctgagaaggccagccctccctggctgtgagcagcctccactgtgtccagagacatgggcctcccactcctgttccttgctagccctggggtggcgtctgcctaggagctggctggcaggtgttgggacctgctgctccatggatgcatgccctaagagtgtcactgagctgtgttttgtctgagcctctctcggtcaacagcaaagcttggtgtcttggcactgttagtgacagagcccagcatcccttctgcccccgttccagctgacatcttgcacggtgaccccttttagtcaggagagtgcagatctgtgctcatcggagactgccccacggccctgtcagagccgccactcctatccccaggccaggtccctggaccagcctcctgtttgcaggcccagaggagccaagtcattaaaatggaagtggattctggatggccgggctgctgctgatgtaggagctggatttgggagctctgcttgccgactggctgtgagacgaggcaggggctctgcttcctcagccctagaggcgagccaggcaaggttggcgactgtcatgtggcttggtttggtcatgcccgtcgatgttttgggtattgaatgtggtaagtggaggaaatgttggaactctgtgcaggtgctgccttgagacccccaagcttccacctgtccctctcctatgtggcagctggggagcagctgagatgtggacttgtatgctgcccacatacgtgagggggagctgaaagggagcccctcctctgagcagcctctgccaggcctgtatgaggcttttcccaccagctcccaacagaggcctcccccagccaggaccacctcgtcctcgtggcggggcagcaggagcggtagaaaggggtccgatgtttgaggaggcccttaagggaagctactgaattataacacgtaagaaaatcaccattccgtattggttgggggctcctgtttctcatcctagctttttcctggaaagcccgctagaaggtttgggaacgaggggaaagttctcagaactgttggctgctccccacccgcctcccgcctcccccgcaggttatgtcagcagctctgagacagcagtatcacaggccagatgttgttcctggctagatgtttacatttgtaagaaataacactgtgaatgtaaaacagagccattcccttggaatgcatatcgctgggctcaacatagagtttgtcttcctcttgtttacgacgtgatctaaaccagtccttagcaaggggctcagaacaccccgctctggcagtaggtgtcccccacccccaaagacctgcctgtgtgctccggagatgaatatgagctcattagtaaaaatgacttcacccacgcatatacataaagtatccatgcatgtgcatatagacacatctataattttacacacacacctctcaagacggagatgcatggcctctaagagtgcccgtgtcggttcttcctggaagttgactttccttagacccgccaggtcaagttagccgcgtgacggacatccaggcgtgggacgtggtcagggcagggctcattcattgcccactaggatcccactggcgaagatggtctccatatcagctctctgcagaagggaggaagactttatcatgttcctaaaaatctgtggcaagcacccatcgtattatccaaattttgttgcaaatgtgattaatttggttgtcaagttttgggggtgggctgtggggagattgcttttgttttcctgctggtaatatcgggaaagattttaatgaaaccagggtagaattgtttggcaatgcactgaagcgtgtttctttcccaaaatgtgcctcccttccgctgcgggcccagctgagtctatgtaggtgatgtttccagctgccaagtgctctttgttactgtccaccctcatttctgccagcgcatgtgtcctttcaaggggaaaatgtgaagctgaaccccctccagacacccagaatgtagcatctgagaaggccctgtgccctaaaggacacccctcgcccccatcttcatggagggggtcatttcagagccctcggagccaatgaacagctcctcctcttggagctgagatgagccccacgtggagctcgggacggatagtagacagcaataactcggtgtgtggccgcctggcaggtggaacttcctcccgttgcggggtggagtgaggttagttctgtgtgtctggtgggtggagtcaggcttctcttgctacctgtgagcatccttcccagcagacatcctcatcgggctttgtccctcccccgcttcctccctctgcggggaggacccgggaccacagctgctggccagggtagacttggagctgtcctccagaggggtcacgtgtaggagtgagaagaaggaagatcttgagagctgctgagggaccttggagagctcaggatggctcagacgaggacactcgcttgccgggcctgggcctcctgggaaggagggagctgctcagaatgccgcatgacaactgaaggcaacctggaaggttcaggggccgctcttcccccatgtgcctgtcacgctctggtgcagtcaaaggaacgccttcccctcagttgtttctaagagcagagtctcccgctgcaatctgggtggtaactgccagccttggaggatcgtggccaacgtggacctgcctacggagggtgggctctgacccaagtggggcctccttgtccaggtctcactgctttgcaccgtggtcagagggactgtcagctgagcttgagctcccctggagccagcagggctgtgatgggcgagtcccggagccccacccagacctgaatgcttctgagagcaaagggaaggactgacgagagatgtatatttaattttttaactgctgcaaacattgtacatccaaattaaaggaaaaaaatggaaaccatca

SEQ ID NO: 3 - DNA, 20 bp, synthetic construct
  source 1..20: mol_type = other DNA
ctccgtccggtagacatgct

SEQ ID NO: 4 - DNA, 23 bp, synthetic construct
  source 1..23: mol_type = other DNA
ggaaatcagaaccctcaaaatgg

SEQ ID NO: 5 - DNA, 29 bp, synthetic construct
  source 1..29: mol_type = other DNA
tgagcactgttcaactgtggatatcggga

SEQ ID NO: 6 - DNA, 20 bp, synthetic construct
  source 1..20: mol_type = other DNA
tctctattgcacattccaag

SEQ ID NO: 7 - DNA, 20 bp, synthetic construct
  source 1..20: mol_type = other DNA
gccgtagcctgggacccgcc

SEQ ID NO: 8 - DNA, 19 bp, synthetic construct
  source 1..19: mol_type = other DNA
taacactcgattaaccctg

SEQ ID NO: 9 - DNA, 19 bp, synthetic construct
  source 1..19: mol_type = other DNA
taacacttgattaaccctg

SEQ ID NO: 10 - DNA, 19 bp, synthetic construct
  source 1..19: mol_type = other DNA
gttaacactcgattaaccc

SEQ ID NO: 11 - DNA, 19 bp, synthetic construct
  source 1..19: mol_type = other DNA
gttaacacttgattaaccc

SEQ ID NO: 12 - DNA, 19 bp, synthetic construct
  source 1..19: mol_type = other DNA
gctagttcatcccagtgag

SEQ ID NO: 13 - DNA, 19 bp, synthetic construct
  source 1..19: mol_type = other DNA
gctagttcaccccagtgag

SEQ ID NO: 14 - DNA, 19 bp, synthetic construct
  source 1..19: mol_type = other DNA
tggaaatgggtttttccac

SEQ ID NO: 15 - DNA, 19 bp, synthetic construct
  source 1..19: mol_type = other DNA
tggaaatggctttttccac

SEQ ID NO: 16 - DNA, 19 bp, synthetic construct
  source 1..19: mol_type = other DNA
tttaaccgtggcatgggca

SEQ ID NO: 17 - DNA, 19 bp, synthetic construct
  source 1..19: mol_type = other DNA
tttaaccgtagcatgggca

SEQ ID NO: 18 - DNA, 19 bp, synthetic construct
  source 1..19: mol_type = other DNA
ttcaagctagtaacgatgc

SEQ ID NO: 19 - DNA, 19 bp, synthetic construct
  source 1..19: mol_type = other DNA
ttcaagccagtaacgatgc

SEQ ID NO: 20 - DNA, 19 bp, synthetic construct
  source 1..19: mol_type = other DNA
acttcaagctagtaacgat

SEQ ID NO: 21 - DNA, 19 bp, synthetic construct
  source 1..19: mol_type = other DNA
acttcaagccagtaacgat

SEQ ID NO: 22 - DNA, 19 bp, synthetic construct
  source 1..19: mol_type = other DNA
gcagctaggttaaagagtc

SEQ ID NO: 23 - DNA, 19 bp, synthetic construct
  source 1..19: mol_type = other DNA
gcagctaggctaaagagtc

SEQ ID NO: 24 - DNA, 19 bp, synthetic construct
  source 1..19: mol_type = other DNA
aataagaaacacaatcaaa

SEQ ID NO: 25 - DNA, 19 bp, synthetic construct
  source 1..19: mol_type = other DNA
aataagaaatacaatcaaa

SEQ ID NO: 26 - DNA, 19 bp, synthetic construct
  source 1..19: mol_type = other DNA
cagaggaggcatactgtat

SEQ ID NO: 27 - DNA, 19 bp, synthetic construct
  source 1..19: mol_type = other DNA
cagaggaggtatactgtat

SEQ ID NO: 28 - DNA, 19 bp, synthetic construct
  source 1..19: mol_type = other DNA
cacagtgctacccaacctt

SEQ ID NO: 29 - DNA, 19 bp, synthetic construct
  source 1..19: mol_type = other DNA
cacagtgctccccaacctt

SEQ ID NO: 30 - DNA, 19 bp, synthetic construct
  source 1..19: mol_type = other DNA
taattttctagactttatg

SEQ ID NO: 31 - DNA, 19 bp, synthetic construct
  source 1..19: mol_type = other DNA
taattttctggactttatg

SEQ ID NO: 32 - DNA, 19 bp, synthetic construct
  source 1..19: mol_type = other DNA
gctacaacgcaggtcaaat

SEQ ID NO: 33 - DNA, 19 bp, synthetic construct
  source 1..19: mol_type = other DNA
gctacaatgcaggtcaaat

SEQ ID NO: 34 - DNA, 19 bp, synthetic construct
  source 1..19: mol_type = other DNA
gagctacaacgcaggtcaa

SEQ ID NO: 35 - DNA, 19 bp, synthetic construct
  source 1..19: mol_type = other DNA
gagctacaatgcaggtcaa

SEQ ID NO: 36 - DNA, 19 bp, synthetic construct
  source 1..19: mol_type = other DNA
agagagaacgagaaggctc

SEQ ID NO: 37 - DNA, 19 bp, synthetic construct
  source 1..19: mol_type = other DNA
agagagaacaagaaggctc

SEQ ID NO: 38 - DNA, 19 bp, synthetic construct
  source 1..19: mol_type = other DNA
agcccctctgtgtaagttt

SEQ ID NO: 39 - DNA, 19 bp, synthetic construct
  source 1..19: mol_type = other DNA
agcccttctgtgtaagttt

SEQ ID NO: 40 - DNA, 19 bp, synthetic construct
  source 1..19: mol_type = other DNA
gagcccctctgtgtaagtt

SEQ ID NO: 41 - DNA, 19 bp, synthetic construct
  source 1..19: mol_type = other DNA
gagcccttctgtgtaagtt

SEQ ID NO: 42 - DNA, 19 bp, synthetic construct
  source 1..19: mol_type = other DNA
tgagcccctctgtgtaagt

SEQ ID NO: 43 - DNA, 19 bp, synthetic construct
  source 1..19: mol_type = other DNA
tgagcccttctgtgtaagt

SEQ ID NO: 44 - DNA, 19 bp, synthetic construct
  source 1..19: mol_type = other DNA
atgagcccctctgtgtaag

SEQ ID NO: 45 - DNA, 19 bp, synthetic construct
  source 1..19: mol_type = other DNA
atgagcccttctgtgtaag

SEQ ID NO: 46 - DNA, 19 bp, synthetic construct
  source 1..19: mol_type = other DNA
gatgagcccctctgtgtaa

SEQ ID NO: 47 - DNA, 19 bp, synthetic construct
  source 1..19: mol_type = other DNA
gatgagcccttctgtgtaa

SEQ ID NO: 48 - DNA, 19 bp, synthetic construct
  source 1..19: mol_type = other DNA
tgatgagcccctctgtgta

SEQ ID NO: 49 - DNA, 19 bp, synthetic construct
  source 1..19: mol_type = other DNA
tgatgagcccttctgtgta

SEQ ID NO: 50 - DNA, 19 bp, synthetic construct
  source 1..19: mol_type = other DNA
atgatgagcccctctgtgt

SEQ ID NO: 51 - DNA, 19 bp, synthetic construct
  source 1..19: mol_type = other DNA
atgatgagcccttctgtgt

SEQ ID NO: 52 - DNA, 19 bp, synthetic construct
  source 1..19: mol_type = other DNA
taatgatgagcccctctgt

SEQ ID NO: 53 - DNA, 19 bp, synthetic construct
  source 1..19: mol_type = other DNA
taatgatgagcccttctgt

SEQ ID NO: 54 - DNA, 19 bp, synthetic construct
  source 1..19: mol_type = other DNA
agaatacgggtaacatttt

SEQ ID NO: 55 - DNA, 19 bp, synthetic construct
  source 1..19: mol_type = other DNA
agaatacaggtaacatttt

SEQ ID NO: 56 - DNA, 19 bp, synthetic construct
  source 1..19: mol_type = other DNA
ggagaatacgggtaacatt

SEQ ID NO: 57 - DNA, 19 bp, synthetic construct
  source 1..19: mol_type = other DNA
ggagaatacaggtaacatt

SEQ ID NO: 58 - DNA, 19 bp, synthetic construct
  source 1..19: mol_type = other DNA
ttagtaatcaattttaatg

SEQ ID NO: 59 - DNA, 19 bp, synthetic construct
  source 1..19: mol_type = other DNA
ttagtaaccaattttaatg

SEQ ID NO: 60 - DNA, 19 bp, synthetic construct
  source 1..19: mol_type = other DNA
agttagtaatcaattttaa

SEQ ID NO: 61 - DNA, 19 bp, synthetic construct
  source 1..19: mol_type = other DNA
agttagtaaccaattttaa

SEQ ID NO: 62 - DNA, 19 bp, synthetic construct
  source 1..19: mol_type = other DNA
gaaggaatgcttttactag

SEQ ID NO: 63 - DNA, 19 bp, synthetic construct
  source 1..19: mol_type = other DNA
gaaggaatgtttttactag

SEQ ID NO: 64 - DNA, 19 bp, synthetic construct
  source 1..19: mol_type = other DNA
ctaaaactaacttgagaat

SEQ ID NO: 65 - DNA, 19 bp, synthetic construct
  source 1..19: mol_type = other DNA
ctaaaaccaacttgagaat

SEQ ID NO: 66 - DNA, 19 bp, synthetic construct
  source 1..19: mol_type = other DNA
atctaaaactaacttgaga

SEQ ID NO: 67 - DNA, 19 bp, synthetic construct
  source 1..19: mol_type = other DNA
atctaaaaccaacttgaga

SEQ ID NO: 68 - DNA, 19 bp, synthetic construct
  source 1..19: mol_type = other DNA
ggtgggcaggaaggactga

SEQ ID NO: 69 - DNA, 19 bp, synthetic construct
  source 1..19: mol_type = other DNA
ggtgggcagaaaggactga

SEQ ID NO: 70 - DNA, 19 bp, synthetic construct
  source 1..19: mol_type = other DNA
cctaaatcaatctacaagt

SEQ ID NO: 71 - DNA, 19 bp, synthetic construct
  source 1..19: mol_type = other DNA
cctaaattaatctacaagt

SEQ ID NO: 72 - DNA, 19 bp, synthetic construct
  source 1..19: mol_type = other DNA
tccctaaatcaatctacaa

SEQ ID NO: 73 - DNA, 19 bp, synthetic construct
  source 1..19: mol_type = other DNA
tccctaaattaatctacaa

SEQ ID NO: 74 - DNA, 19 bp, synthetic construct
  source 1..19: mol_type = other DNA
gaaaatgtgagtggatcta

SEQ ID NO: 75 - DNA, 19 bp, synthetic construct
  source 1..19: mol_type = other DNA
gaaaatgtgcgtggatcta

SEQ ID NO: 76 - DNA, 19 bp, synthetic construct
  source 1..19: mol_type = other DNA
gtaaggcgagactgactag

SEQ ID NO: 77 - DNA, 19 bp, synthetic construct
  source 1..19: mol_type = other DNA
gtaaggcaagactgactag

SEQ ID NO: 78 - DNA, 19 bp, synthetic construct
  source 1..19: mol_type = other DNA
aggtaaggcgagactgact

SEQ ID NO: 79 - DNA, 19 bp, synthetic construct
  source 1..19: mol_type = other DNA
aggtaaggcaagactgact

SEQ ID NO: 80 - DNA, 19 bp, synthetic construct
  source 1..19: mol_type = other DNA
ctgagcggagaaaccctcc

SEQ ID NO: 81 - DNA, 19 bp, synthetic construct
  source 1..19: mol_type = other DNA
ctgagcgaagaaaccctcc

SEQ ID NO: 82 - DNA, 19 bp, synthetic construct
  source 1..19: mol_type = other DNA
ggctgagcggagaaaccct

SEQ ID NO: 83 - DNA, 19 bp, synthetic construct
  source 1..19: mol_type = other DNA
ggctgagcgaagaaaccct

SEQ ID NO: 84 - DNA, 19 bp, synthetic construct
  source 1..19: mol_type = other DNA
aaggctgagcggagaaacc

SEQ ID NO: 85 - DNA, 19 bp, synthetic construct
  source 1..19: mol_type = other DNA
ttccctaaaaacaaaaaca

SEQ ID NO: 86 - DNA, 19 bp, synthetic construct
  source 1..19: mol_type = other DNA
ttccctagaaacaaaaaca

SEQ ID NO: 87 - DNA, 19 bp, synthetic construct
  source 1..19: mol_type = other DNA
gattccctaaaaacaaaaa

SEQ ID NO: 88 - DNA, 19 bp, synthetic construct
  source 1..19: mol_type = other DNA
gattccctagaaacaaaaa

SEQ ID NO: 89 - DNA, 19 bp, synthetic construct
  source 1..19: mol_type = other DNA
cttttctattgtctgtccc

SEQ ID NO: 90 - DNA, 19 bp, synthetic construct
  source 1..19: mol_type = other DNA
cttttctgttgtctgtccc

SEQ ID NO: 91 - DNA, 19 bp, synthetic construct
  source 1..19: mol_type = other DNA
tgcttttctattgtctgtc

SEQ ID NO: 92 - DNA, 19 bp, synthetic construct
  source 1..19: mol_type = other DNA
tgcttttctgttgtctgtc

SEQ ID NO: 93 - DNA, 19 bp, synthetic construct
  source 1..19: mol_type = other DNA
aagggatgccgacttgggc

SEQ ID NO: 94 - DNA, 19 bp, synthetic construct
  source 1..19: mol_type = other DNA
aagggatgctgacttgggc

SEQ ID NO: 95 - DNA, 19 bp, synthetic construct
  source 1..19: mol_type = other DNA
accttcctcactgaggatg

SEQ ID NO: 96 - DNA, 19 bp, synthetic construct
  source 1..19: mol_type = other DNA
accttcctcgctgaggatg

SEQ ID NO: 97 - DNA, 19 bp, synthetic construct
  source 1..19: mol_type = other DNA
caaaccactgtgggatgaa

SEQ ID NO: 98 - DNA, 19 bp, synthetic construct
  source 1..19: mol_type = other DNA
caaaccactttgggatgaa

SEQ ID NO: 99 - DNA, 19 bp, synthetic construct
  source 1..19: mol_type = other DNA
aataaattgtcatcaccag

SEQ ID NO: 100 - DNA, 19 bp, synthetic construct
  source 1..19: mol_type = other DNA
aataaattgccatcaccag

SEQ ID NO: 101 - DNA, 19 bp, synthetic construct
  source 1..19: mol_type = other DNA
tcacagctatcttctcatc

SEQ ID NO: 102 - DNA, 19 bp, synthetic construct
  source 1..19: mol_type = other DNA
tcacagctaacttctcatc

SEQ ID NO: 103 - DNA, 19 bp, synthetic construct
  source 1..19: mol_type = other DNA
gcacacagtagatgaggga

SEQ ID NO: 104 - DNA, 19 bp, synthetic construct
  source 1..19: mol_type = other DNA
gcacacagtggatgaggga

SEQ ID NO: 105 - DNA, 19 bp, synthetic construct
  source 1..19: mol_type = other DNA
cagaacaaagagaagaatt

SEQ ID NO: 106 - DNA, 19 bp, synthetic construct
  source 1..19: mol_type = other DNA
cagaacaaacagaagaatt

SEQ ID NO: 107 - DNA, 19 bp, synthetic construct
  source 1..19: mol_type = other DNA
gcttacatgccttcagtga

SEQ ID NO: 108 - DNA, 19 bp, synthetic construct
  source 1..19: mol_type = other DNA
gcttacacgccttcagtga

SEQ ID NO: 109 - DNA, 19 bp, synthetic construct
  source 1..19: mol_type = other DNA
cagcttacatgccttcagt

SEQ ID NO: 110 - DNA, 19 bp, synthetic construct
  source 1..19: mol_type = other DNA
cagcttacacgccttcagt

SEQ ID NO: 111 - DNA, 19 bp, synthetic construct
  source 1..19: mol_type = other DNA
aagaagcctgataaaatct

SEQ ID NO: 112 - DNA, 19 bp, synthetic construct
  source 1..19: mol_type = other DNA
aagaagcctaataaaatct

SEQ ID NO: 113 - DNA, 19 bp, synthetic construct
  source 1..19: mol_type = other DNA
catacatcagctcaaactg

SEQ ID NO: 114 - DNA, 19 bp, synthetic construct
  source 1..19: mol_type = other DNA
catacattagctcaaactg

SEQ ID NO: 115 - DNA, 19 bp, synthetic construct
  source 1..19: mol_type = other DNA
cacatacatcagctcaaac

SEQ ID NO: 116 - DNA, 19 bp, synthetic construct
  source 1..19: mol_type = other DNA
cacatacattagctcaaac

SEQ ID NO: 117 - DNA, 19 bp, synthetic construct
  source 1..19: mol_type = other DNA
gtcacatacatcagctcaa

SEQ ID NO: 118 - DNA, 19 bp, synthetic construct
  source 1..19: mol_type = other DNA
gagactatagcacccagat

SEQ ID NO: 119 - DNA, 19 bp, synthetic construct
  source 1..19: mol_type = other DNA
gagactataacacccagat

SEQ ID NO: 120 - DNA, 19 bp, synthetic construct
  source 1..19: mol_type = other DNA
tagaggacgccgtgcaggg

SEQ ID NO: 121 - DNA, 19 bp, synthetic construct
  source 1..19: mol_type = other DNA
tagaggatgccgtgcaggg

SEQ ID NO: 122 - DNA, 19 bp, synthetic construct
  source 1..19: mol_type = other DNA
catagaggacgccgtgcag

SEQ ID NO: 123 - DNA, 19 bp, synthetic construct
  source 1..19: mol_type = other DNA
catagaggatgccgtgcag

SEQ ID NO: 124 - DNA, 19 bp, synthetic construct
  source 1..19: mol_type = other DNA
cacatagaggacgccgtgc

SEQ ID NO: 125 - DNA, 19 bp, synthetic construct
  source 1..19: mol_type = other DNA
acgtgtgtacagaacctgc

SEQ ID NO: 126 - DNA, 19 bp, synthetic construct
  source 1..19: mol_type = other DNA
acgtgtgtatagaacctgc

SEQ ID NO: 127 - DNA, 19 bp, synthetic construct
  source 1..19: mol_type = other DNA
tgttcagaatgcctcatct

SEQ ID NO: 128 - DNA, 19 bp, synthetic construct
  source 1..19: mol_type = other DNA
tgttcagaacgcctcatct

SEQ ID NO: 129 - DNA, 19 bp, synthetic construct
  source 1..19: mol_type = other DNA
aaacggcgcagcgggaagg

SEQ ID NO: 130 - DNA, 19 bp, synthetic construct
  source 1..19: mol_type = other DNA
aaacggcacagcgggaagg

SEQ ID NO: 131 - DNA, 19 bp, synthetic construct
  source 1..19: mol_type = other DNA
agaaacggcgcagcgggaa

SEQ ID NO: 132 - DNA, 19 bp, synthetic construct
  source 1..19: mol_type = other DNA
agaaacggcacagcgggaa

SEQ ID NO: 133 - DNA, 19 bp, synthetic construct
  source 1..19: mol_type = other DNA
agggcgcagacttccaaag

SEQ ID NO: 134 - DNA, 19 bp, synthetic construct
  source 1..19: mol_type = other DNA
agggcacagacttccaaag

SEQ ID NO: 135 - DNA, 19 bp, synthetic construct
  source 1..19: mol_type = other DNA
aagggcgcagacttccaaa

SEQ ID NO: 136 - DNA, 19 bp, synthetic construct
  source 1..19: mol_type = other DNA
aagggcacagacttccaaa

SEQ ID NO: 137 - DNA, 19 bp, synthetic construct
  source 1..19: mol_type = other DNA
caagggcgcagacttccaa

SEQ ID NO: 138 - DNA, 19 bp, synthetic construct
  source 1..19: mol_type = other DNA
caagggcacagacttccaa

SEQ ID NO: 139 - DNA, 19 bp, synthetic construct
  source 1..19: mol_type = other DNA
acaagggcgcagacttcca

SEQ ID NO: 140 - DNA, 19 bp, synthetic construct
  source 1..19: mol_type = other DNA
acaagggcacagacttcca

SEQ ID NO: 141 - DNA, 19 bp, synthetic construct
  source 1..19: mol_type = other DNA
cacaagggcgcagacttcc

SEQ ID NO: 142 - DNA, 19 bp, synthetic construct
  source 1..19: mol_type = other DNA
cacaagggcacagacttcc

SEQ ID NO: 143 - DNA, 19 bp, synthetic construct
  source 1..19: mol_type = other DNA
gcacaagggcgcagacttc

SEQ ID NO: 144 - DNA, 19 bp, synthetic construct
  source 1..19: mol_type = other DNA
gcacaagggcacagacttc

SEQ ID NO: 145 - DNA, 19 bp, synthetic construct
  source 1..19: mol_type = other DNA
ggcacaagggcgcagactt

SEQ ID NO: 146 - DNA, 19 bp, synthetic construct
  source 1..19: mol_type = other DNA
ggcacaagggcacagactt

SEQ ID NO: 147 - DNA, 19 bp, synthetic construct
  source 1..19: mol_type = other DNA
agggcacaagggcgcagac

SEQ ID NO: 148 - DNA, 19 bp, synthetic construct
  source 1..19: mol_type = other DNA
agggcacaagggcacagac

SEQ ID NO: 149 - DNA, 19 bp, synthetic construct
  source 1..19: mol_type = other DNA
gagcagctgcaacctggca

SEQ ID NO: 150 - DNA, 19 bp, synthetic construct
  source 1..19: mol_type = other DNA
gagcagctgtaacctggca

SEQ ID NO: 151 - DNA, 19 bp, synthetic construct
  source 1..19: mol_type = other DNA
tggtgccgggtgtctagca

SEQ ID NO: 152 - DNA, 19 bp, synthetic construct
  source 1..19: mol_type = other DNA
tggtgccaggtgtctagca

SEQ ID NO: 153 - DNA, 19 bp, synthetic construct
  source 1..19: mol_type = other DNA
aatggtgccgggtgtctag

SEQ ID NO: 154 - DNA, 19 bp, synthetic construct
  source 1..19: mol_type = other DNA
aatggtgccaggtgtctag

SEQ ID NO: 155 - DNA, 19 bp, synthetic construct
  source 1..19: mol_type = other DNA
ggggacagggtgtgctctc

SEQ ID NO: 156 - DNA, 19 bp, synthetic construct
  source 1..19: mol_type = other DNA
ggggacaggttgtgctctc

SEQ ID NO: 157 - DNA, 19 bp, synthetic construct
  source 1..19: mol_type = other DNA
gcttttcattgaaaagaaa

SEQ ID NO: 158 - DNA, 19 bp, synthetic construct
  source 1..19: mol_type = other DNA
gcttttcgttgaaaagaaa

SEQ ID NO: 159 - DNA, 19 bp, synthetic construct
  source 1..19: mol_type = other DNA
ctgcttttcattgaaaaga

SEQ ID NO: 160 - DNA, 19 bp, synthetic construct
  source 1..19: mol_type = other DNA
ctgcttttcgttgaaaaga

SEQ ID NO: 161 - DNA, 19 bp, synthetic construct
  source 1..19: mol_type = other DNA
actaggccgggcatgctgg

SEQ ID NO: 162 - DNA, 19 bp, synthetic construct
  source 1..19: mol_type = other DNA
actaggctgggcatgctgg

SEQ ID NO: 163 - DNA, 19 bp, synthetic construct
  source 1..19: mol_type = other DNA
agactaggccgggcatgct

SEQ ID NO: 164 - DNA, 19 bp, synthetic construct
  source 1..19: mol_type = other DNA
agactaggctgggcatgct

SEQ ID NO: 165 - DNA, 19 bp, synthetic construct
  source 1..19: mol_type = other DNA
aaacagctgttagttccca

SEQ ID NO: 166 - DNA, 19 bp, synthetic construct
  source 1..19: mol_type = other DNA
aaacagccgttagttccca

SEQ ID NO: 167 - DNA, 19 bp, synthetic construct
  source 1..19: mol_type = other DNA
agaaacagctgttagttcc

SEQ ID NO: 168 - DNA, 19 bp, synthetic construct
  source 1..19: mol_type = other DNA
agaaacagccgttagttcc

SEQ ID NO: 169 - DNA, 19 bp, synthetic construct
  source 1..19: mol_type = other DNA
gtgctacccaacctttctg

SEQ ID NO: 170 - DNA, 19 bp, synthetic construct
  source 1..19: mol_type = other DNA
agtgctacccaacctttct

SEQ ID NO: 171 - DNA, 19 bp, synthetic construct
  source 1..19: mol_type = other DNA
cagtgctacccaacctttc

SEQ ID NO: 172 - DNA, 19 bp, synthetic construct
  source 1..19: mol_type = other DNA
acagtgctacccaaccttt

SEQ ID NO: 173 - DNA, 18 bp, synthetic construct
  source 1..18: mol_type = other DNA
acagtgctacccaacctt

SEQ ID NO: 174 - DNA, 17 bp, synthetic construct
  source 1..17: mol_type = other DNA
acagtgctacccaacct

SEQ ID NO: 175 - DNA, 16 bp, synthetic construct
  source 1..16: mol_type = other DNA
cagtgctacccaacct

SEQ ID NO: 176 - DNA, 19 bp, synthetic construct
  source 1..19: mol_type = other DNA
tcacagtgctacccaacct

SEQ ID NO: 177 - DNA, 15 bp, synthetic construct
  source 1..15: mol_type = other DNA
cagtgctacccaacc

SEQ ID NO: 178 - DNA, 19 bp, synthetic construct
  source 1..19: mol_type = other DNA
atcacagtgctacccaacc

SEQ ID NO: 179 - DNA, 19 bp, synthetic construct
  source 1..19: mol_type = other DNA
tatcacagtgctacccaac

SEQ ID NO: 180 - DNA, 19 bp, synthetic construct
  source 1..19: mol_type = other DNA
atatcacagtgctacccaa

SEQ ID NO: 181 - DNA, 19 bp, synthetic construct
  source 1..19: mol_type = other DNA
atgctgacttgggccattc

SEQ ID NO: 182 - DNA, 19 bp, synthetic construct
  source 1..19: mol_type = other DNA
gatgctgacttgggccatt

SEQ ID NO: 183 - DNA, 19 bp, synthetic construct
  source 1..19: mol_type = other DNA
ggatgctgacttgggccat

SEQ ID NO: 184 - DNA, 19 bp, synthetic construct
  source 1..19: mol_type = other DNA
gggatgctgacttgggcca

SEQ ID NO: 185 - DNA, 19 bp, synthetic construct
  source 1..19: mol_type = other DNA
agggatgctgacttgggcc

SEQ ID NO: 186 - DNA, 18 bp, synthetic construct
  source 1..18: mol_type = other DNA
agggatgctgacttgggc

SEQ ID NO: 187 - DNA, 17 bp, synthetic construct
  source 1..17: mol_type = other DNA
agggatgctgacttggg

SEQ ID NO: 188 - DNA, 19 bp, synthetic construct
  source 1..19: mol_type = other DNA
caagggatgctgacttggg

SEQ ID NO: 189 - DNA, 15 bp, synthetic construct
  source 1..15: mol_type = other DNA
gggatgctgacttgg

SEQ ID NO: 190 - DNA, 19 bp, synthetic construct
  source 1..19: mol_type = other DNA
ccaagggatgctgacttgg

SEQ ID NO: 191 - DNA, 19 bp, synthetic construct
  source 1..19: mol_type = other DNA
gccaagggatgctgacttg

SEQ ID NO: 192 - DNA, 19 bp, synthetic construct
  source 1..19: mol_type = other DNA
tgccaagggatgctgactt

SEQ ID NO: 193 - DNA, 19 bp, synthetic construct
  source 1..19: mol_type = other DNA
ctgccaagggatgctgact

SEQ ID NO: 194 - DNA, 19 bp, synthetic construct
  source 1..19: mol_type = other DNA
attgtcatcaccagaaaaa

SEQ ID NO: 195 - DNA, 19 bp, synthetic construct
  source 1..19: mol_type = other DNA
aattgtcatcaccagaaaa

SEQ ID NO: 196 - DNA, 19 bp, synthetic construct
  source 1..19: mol_type = other DNA
aaattgtcatcaccagaaa

SEQ ID NO: 197 - DNA, 19 bp, synthetic construct
  source 1..19: mol_type = other DNA
taaattgtcatcaccagaa

SEQ ID NO: 198 - DNA, 19 bp, synthetic construct
  source 1..19: mol_type = other DNA
ataaattgtcatcaccaga

SEQ ID NO: 199 - DNA, 18 bp, synthetic construct
  source 1..18: mol_type = other DNA
ataaattgtcatcaccag

SEQ ID NO: 200 - DNA, 17 bp, synthetic construct
  source 1..17: mol_type = other DNA
ataaattgtcatcacca

SEQ ID NO: 201 - DNA, 16 bp, synthetic construct
  source 1..16: mol_type = other DNA
taaattgtcatcacca

SEQ ID NO: 202 - DNA, 19 bp, synthetic construct
  source 1..19: mol_type = other DNA
taataaattgtcatcacca

SEQ ID NO: 203 - DNA, 15 bp, synthetic construct
  source 1..15: mol_type = other DNA
taaattgtcatcacc

SEQ ID NO: 204 - DNA, 19 bp, synthetic construct
  source 1..19: mol_type = other DNA
ttaataaattgtcatcacc

SEQ ID NO: 205 - DNA, 19 bp, synthetic construct
  source 1..19: mol_type = other DNA
attaataaattgtcatcac

SEQ ID NO: 206 - DNA, 19 bp, synthetic construct
  source 1..19: mol_type = other DNA
tattaataaattgtcatca

SEQ ID NO: 207 - DNA, 19 bp, synthetic construct
  source 1..19: mol_type = other DNA
ctattaataaattgtcatc

SEQ ID NO: 208 - DNA, 19 bp, synthetic construct
  source 1..19: mol_type = other DNA
cagtagatgagggagcagg

SEQ ID NO: 209 - DNA, 19 bp, synthetic construct
  source 1..19: mol_type = other DNA
acagtagatgagggagcag

SEQ ID NO: 210 - DNA, 19 bp, synthetic construct
  source 1..19: mol_type = other DNA
cacagtagatgagggagca

SEQ ID NO: 211 - DNA, 19 bp, synthetic construct
  source 1..19: mol_type = other DNA
acacagtagatgagggagc

SEQ ID NO: 212 - DNA, 19 bp, synthetic construct
  source 1..19: mol_type = other DNA
cacacagtagatgagggag

SEQ ID NO: 213 - DNA, 18 bp, synthetic construct
  source 1..18: mol_type = other DNA
cacacagtagatgaggga

SEQ ID NO: 214 - DNA, 17 bp, synthetic construct
  source 1..17: mol_type = other DNA
cacacagtagatgaggg

SEQ ID NO: 215 - DNA, 16 bp, synthetic construct
  source 1..16: mol_type = other DNA
acacagtagatgaggg

SEQ ID NO: 216 - DNA, 19 bp, synthetic construct
  source 1..19: mol_type = other DNA
tgcacacagtagatgaggg

SEQ ID NO: 217 - DNA, 15 bp, synthetic construct
  source 1..15: mol_type = other DNA
acacagtagatgagg

SEQ ID NO: 218 - DNA, 19 bp, synthetic construct
  source 1..19: mol_type = other DNA
gtgcacacagtagatgagg

SEQ ID NO: 219 - DNA, 19 bp, synthetic construct
  source 1..19: mol_type = other DNA
agtgcacacagtagatgag

SEQ ID NO: 220 - DNA, 19 bp, synthetic construct
  source 1..19: mol_type = other DNA
aagtgcacacagtagatga

SEQ ID NO: 221 - DNA, 19 bp, synthetic construct
  source 1..19: mol_type = other DNA
gaagtgcacacagtagatg

SEQ ID NO: 222 - DNA, 19 bp, synthetic construct
  source 1..19: mol_type = other DNA
gctgcaacctggcaacaac

SEQ ID NO: 223 - DNA, 19 bp, synthetic construct
  source 1..19: mol_type = other DNA
agctgcaacctggcaacaa

SEQ ID NO: 224 - DNA, 19 bp, synthetic construct
  source 1..19: mol_type = other DNA
cagctgcaacctggcaaca

SEQ ID NO: 225 - DNA, 19 bp, synthetic construct
  source 1..19: mol_type = other DNA
gcagctgcaacctggcaac

SEQ ID NO: 226 - DNA, 19 bp, synthetic construct
  source 1..19: mol_type = other DNA
agcagctgcaacctggcaa

SEQ ID NO: 227 - DNA, 18 bp, synthetic construct
  source 1..18: mol_type = other DNA
agcagctgcaacctggca

SEQ ID NO: 228 - DNA, 17 bp, synthetic construct
  source 1..17: mol_type = other DNA
agcagctgcaacctggc

SEQ ID NO: 229 - DNA, 16 bp, synthetic construct
  source 1..16: mol_type = other DNA
gcagctgcaacctggc

SEQ ID NO: 230 - DNA, 19 bp, synthetic construct
  source 1..19: mol_type = other DNA
agagcagctgcaacctggc

SEQ ID NO: 231 - DNA, 15 bp, synthetic construct
  source 1..15: mol_type = other DNA
gcagctgcaacctgg

SEQ ID NO: 232 - DNA, 19 bp, synthetic construct
  source 1..19: mol_type = other DNA
aagagcagctgcaacctgg

SEQ ID NO: 233 - DNA, 19 bp, synthetic construct
  source 1..19: mol_type = other DNA
caagagcagctgcaacctg

SEQ ID NO: 234 - DNA, 19 bp, synthetic construct
  source 1..19: mol_type = other DNA
gcaagagcagctgcaacct

SEQ ID NO: 235 - DNA, 19 bp, synthetic construct
  source 1..19: mol_type = other DNA
tgcaagagcagctgcaacc

SEQ ID NO: 236 - DNA, 19 bp, synthetic construct
  source 1..19: mol_type = other DNA
accatgatatctccagcac

SEQ ID NO: 237 - DNA, 19 bp, synthetic construct
  source 1..19: mol_type = other DNA
accatgacatctccagcac

SEQ ID NO: 238 - DNA, 19 bp, synthetic construct
  source 1..19: mol_type = other DNA
ccaccatgatatctccagc

SEQ ID NO: 239 - DNA, 19 bp, synthetic construct
  source 1..19: mol_type = other DNA
ccaccatgacatctccagc

SEQ ID NO: 240 - DNA, 19 bp, synthetic construct
  source 1..19: mol_type = other DNA
ttaacactcgattaaccct

SEQ ID NO: 241 - DNA, 19 bp, synthetic construct
  source 1..19: mol_type = other DNA
tagttcatcccagtgagaa

SEQ ID NO: 242 - DNA, 19 bp, synthetic construct
  source 1..19: mol_type = other DNA
ctagttcatcccagtgaga

SEQ ID NO: 243 - DNA, 19 bp, synthetic construct
  source 1..19: mol_type = other DNA
gaaatgggtttttccacat

SEQ ID NO: 244 - DNA, 19 bp, synthetic construct
  source 1..19: mol_type = other DNA
ggaaatgggtttttccaca

SEQ ID NO: 245 - DNA, 19 bp, synthetic construct
  source 1..19: mol_type = other DNA
taaccgtggcatgggcagt

SEQ ID NO: 246 - DNA, 19 bp, synthetic construct
  source 1..19: mol_type = other DNA
ttaaccgtggcatgggcag

SEQ ID NO: 247 - DNA, 19 bp, synthetic construct
  source 1..19: mol_type = other DNA
cttcaagctagtaacgatg

SEQ ID NO: 248 - DNA, 19 bp, synthetic construct
  source 1..19: mol_type = other DNA
agctaggttaaagagtcac

SEQ ID NO: 249 - DNA, 19 bp, synthetic construct
  source 1..19: mol_type = other DNA
cagctaggttaaagagtca

SEQ ID NO: 250 - DNA, 19 bp, synthetic construct
  source 1..19: mol_type = other DNA
taagaaacacaatcaaaga

SEQ ID NO: 251 - DNA, 19 bp, synthetic construct
  source 1..19: mol_type = other DNA
ataagaaacacaatcaaag

SEQ ID NO: 252 - DNA, 19 bp, synthetic construct
  source 1..19: mol_type = other DNA
attttctagactttatgat

SEQ ID NO: 253 - DNA, 19 bp, synthetic construct
  source 1..19: mol_type = other DNA
aattttctagactttatga

SEQ ID NO: 254 - DNA, 19 bp, synthetic construct
  source 1..19: mol_type = other DNA
gagaatacgggtaacattt

SEQ ID NO: 255 - DNA, 19 bp, synthetic construct
  source 1..19: mol_type = other DNA
tgggcaggaaggactgaac

SEQ ID NO: 256 - DNA, 19 bp, synthetic construct
  source 1..19: mol_type = other DNA
gtgggcaggaaggactgaa

SEQ ID NO: 257 - DNA, 19 bp, synthetic construct
  source 1..19: mol_type = other DNA
ccctaaatcaatctacaag

SEQ ID NO: 258 - DNA, 19 bp, synthetic construct
  source 1..19: mol_type = other DNA
cttttccgtgctgttctga

SEQ ID NO: 259 - DNA, 19 bp, synthetic construct
  source 1..19: mol_type = other DNA
acttttccgtgctgttctg

SEQ ID NO: 260 - DNA, 19 bp, synthetic construct
  source 1..19: mol_type = other DNA
aacttttccgtgctgttct

SEQ ID NO: 261 - DNA, 19 bp, synthetic construct
  source 1..19: mol_type = other DNA
gctgagcggagaaaccctc

SEQ ID NO: 262 - DNA, 19 bp, synthetic construct
  source 1..19: mol_type = other DNA
attccctaaaaacaaaaac

SEQ ID NO: 263 - DNA, 19 bp, synthetic construct
  source 1..19: mol_type = other DNA
gcttttctattgtctgtcc

SEQ ID NO: 264 - DNA, 19 bp, synthetic construct
  source 1..19: mol_type = other DNA
cttcctcactgaggatgaa

SEQ ID NO: 265 - DNA, 19 bp, synthetic construct
  source 1..19: mol_type = other DNA
ccttcctcactgaggatga

SEQ ID NO: 266 - DNA, 19 bp, synthetic construct
  source 1..19: mol_type = other DNA
aaccactttgggatgaata

SEQ ID NO: 267 - DNA, 19 bp, synthetic construct
  source 1..19: mol_type = other DNA
aaaccactttgggatgaat

SEQ ID NO: 268 - DNA, 19 bp, synthetic construct
  source 1..19: mol_type = other DNA
acagctatcttctcatcaa

SEQ ID NO: 269 - DNA, 19 bp, synthetic construct
  source 1..19: mol_type = other DNA
cacagctatcttctcatca

SEQ ID NO: 270 - DNA, 19 bp, synthetic construct
  source 1..19: mol_type = other DNA
gaacaaagagaagaatttc

SEQ ID NO: 271 - DNA, 19 bp, synthetic construct
  source 1..19: mol_type = other DNA
agaacaaagagaagaattt

SEQ ID NO: 272 - DNA, 19 bp, synthetic construct
  source 1..19: mol_type = other DNA
gaagcctgataaaatctct

SEQ ID NO: 273 - DNA, 19 bp, synthetic construct
  source 1..19: mol_type = other DNA
agaagcctgataaaatctc

SEQ ID NO: 274 - DNA, 19 bp, synthetic construct
  source 1..19: mol_type = other DNA
tgatctgtagcagcagctt

SEQ ID NO: 275 - DNA, 19 bp, synthetic construct
  source 1..19: mol_type = other DNA
ttgatctgtagcagcagct

SEQ ID NO: 276 - DNA, 19 bp, synthetic construct
  source 1..19: mol_type = other DNA
gttgatctgtagcagcagc

SEQ ID NO: 277 - DNA, 19 bp, synthetic construct
  source 1..19: mol_type = other DNA
atagaggacgccgtgcagg

SEQ ID NO: 278 - DNA, 19 bp, synthetic construct
  source 1..19: mol_type = other DNA
gtgtgtacagaacctgccg

SEQ ID NO: 279 - DNA, 19 bp, synthetic construct
  source 1..19: mol_type = other DNA
cgtgtgtacagaacctgcc

SEQ ID NO: 280 - DNA, 19 bp, synthetic construct
  source 1..19: mol_type = other DNA
ttcagaatgcctcatctgg

SEQ ID NO: 281 - DNA, 19 bp, synthetic construct
  source 1..19: mol_type = other DNA
gttcagaatgcctcatctg

SEQ ID NO: 282 - DNA, 19 bp, synthetic construct
  source 1..19: mol_type = other DNA
ggacagggtgtgctctccg

SEQ ID NO: 283 - DNA, 19 bp, synthetic construct
  source 1..19: mol_type = other DNA
gggacagggtgtgctctcc

SEQ ID NO: 284 - DNA, 16 bp, synthetic construct
  source 1..16: mol_type = other DNA
gggatgctgacttggg

SEQ ID NO: 285 - DNA, 17 bp, synthetic construct
  source 1..17: mol_type = other DNA
acacagtagatgaggga

SEQ ID NO: 286 - RNA, 10081 nt, Mus musculus
  source 1..10081: mol_type = mRNA
gcactcgccgcgagggttgccgggacgggcccaagatggctgagcgccttggttccgcttctgcctgccgcgcagagccccattcattgccttgctgctaagtggcgccgcgtagtgccagtaggctccaagtcttcagggtctgtcccatcgggcaggaagccgtcatggcaaccctggaaaagctgatgaaggctttcgagtcgctcaagtcgtttcagcagcaacagcagcagcagccaccgccgcaggcgccgccgccaccgccgccgccgcctccgcctcaaccccctcagccgccgcctcaggggcagccgccgccgccaccaccgccgctgccaggtccggcagaggaaccgctgcaccgaccaaagaaggaactctcagccaccaagaaagaccgtgtgaatcattgtctaacaatatgtgaaaacattgtggcacagtctctcagaaattctccagaatttcagaaactcttgggcatcgctatggaactgtttctgctgtgcagtgacgatgcggagtcagatgtcagaatggtggctgatgagtgcctcaacaaagtcatcaaagctttgatggattctaatcttccaaggctacagttagaactctataaggaaattaaaaagaatggtgctcctcgaagtttgcgtgctgccctgtggaggtttgctgagctggctcacctggttcgacctcagaagtgcaggccttacctggtgaatcttcttccatgcctgacccgaacaagcaaaagaccggaggaatcagttcaggagaccttggctgcagctgttcctaaaattatggcttcttttggcaatttcgcaaatgacaatgaaattaaggttctgttgaaagctttcatagcaaatctgaagtcaagctctcccaccgtgcggcggacagcagccggctcagccgtgagcatctgccaacattctaggaggacacagtacttctacaactggctccttaatgtcctcctaggtctgctggttcccatggaagaagagcactccactctcctgatcctcggtgtgttgctcacattgaggtgtctagtgcccttgctccagcagcaggtcaaggacacaagtctaaaaggcagctttggggtgacacggaaagaaatggaagtctctccttctacagagcagcttgtccaggtttatgaactgactttgcatcatactcagcaccaagaccacaatgtggtgacaggggcactggagctcctgcagcagctcttccgtacccctccacctgaactcctgcaagcactgaccacaccaggagggcttgggcagctcactctggttcaagaagaggcccggggccgaggccgcagcgggagcatcgtggagcttttagctggagggggttcctcgtgcagccctgtcctctcaagaaagcagaaaggcaaagtgctcttaggagaggaagaagccttggaagatgactcggagtccaggtcagatgtcagcagctcagcctttgcagcctctgtgaagagtgagattggtggagagctcgctgcttcttcaggtgtttccactcctggttctgttggtcacgacatcatcactgagcagcctagatcccagcacacacttcaagcagactctgtggatttgtccggctgtgacctgaccagtgctgctactgatggggatgaggaggacatcttgagccacagctccagccagttcagtgctgtcccatccgaccctgccatggacctgaatgatgggacccaggcctcctcacccatcagtgacagttctcagaccaccactgaaggacctgattcagctgtgactccttcggacagttctgaaattgtgttagatggtgccgatagccagtatttaggcatgcagataggacagccacaggaggacgatgaggagggagctgcaggtgttctttctggtgaagtctcagatgttttcagaaactcttctctggcccttcaacaggcacacttgttggaaagaatgggccatagcaggcagccttccgacagcagtatagataagtatgtaacaagagatgaggttgctgaagccagtgatccagaaagcaagccttgccgaatcaaaggtgacataggacagcctaatgatgatgattctgctcctctggtacattgtgtccgtcttttatctgcttcctttttgttaactggtgaaaagaaagcactggttccagacagagacgtgagagtcagtgtgaaggccctggccctcagctgcattggtgcggctgtggcccttcatccagagtcgttcttcagcagactgtacaaagtacctcttaataccacggaaagtactgaggaacagtatgtttctgacatcttgaactacatcgatcatggagacccacaggtccgaggagctactgccattctctgtgggacccttgtctactccatcctcagtaggtcccgtctccgtgttggtgactggctgggcaacatcagaaccctgacaggaaatacattttctctggtggactgcattcctttactgcagaaaacgttgaaggatgaatcttctgttacttgcaagttggcttgtacagctgtgaggcactgtgtcctgagtctttgcagcagcagctacagtgacttgggattacaactgcttattgatatgctgcctctgaagaacagctcctactggctggtgaggaccgaactgctggacactctggcagagattgacttcaggctcgtgagttttttggaggcaaaagcagaaagtttacaccgaggggctcatcattatacagggtttctaaaactacaagaacgagtactcaataatgtggtcatttatttgcttggagatgaagaccccagggttcgacatgttgctgcaacatcattaacaaggcttgtcccaaagctgttttacaagtgtgaccaaggacaagctgatccagttgtggctgtagcgagggatcagagcagtgtctacctgaagctcctcatgcatgagacccagccaccatcacacttttctgtcagcaccatcaccagaatctatagaggctatagcttactgccaagtataacagatgtcaccatggaaaacaatctctcaagagttgttgccgcagtttctcatgaactcattacgtcaacaacacgggcactcacatttggatgctgtgaagccttgtgtcttctctcagcagcctttccagtttgcacttggagtttaggatggcactgtggagtgcccccactgagtgcctctgatgagtccaggaagagctgcactgttgggatggcctccatgattctcaccttgctttcatcagcttggttcccactggatctctcagcccatcaggatgccttgattttggctggaaacttgctagcagcgagtgcccccaagtctctgagaagttcatggacctctgaagaagaagccaactcagcagccaccagacaggaggaaatctggcctgctctgggggatcggactctagtgcccttggtggagcagcttttctcccacctgctgaaggtgatcaatatctgtgctcatgtcttggacgatgtgactcctggaccagcaatcaaggcagccttgccttctctaacaaaccccccttctctaagtcctattcgacggaaagggaaggagaaagaacctggagaacaagcttctactccaatgagtcccaagaaagttggtgaggccagtgcagcctctcgacaatcagacacctcaggacctgtcacagcaagtaaatcatcctcactggggagtttctaccatctcccctcctacctcaaactgcatgatgtcctgaaagccactcacgccaactataaggtcaccttagatcttcagaacagcactgaaaagtttggggggttcctgcgctctgccttggacgtcctttctcagattctagagctggcgacactgcaggacattggaaagtgtgttgaagaggtccttggatacctgaaatcctgctttagtcgagaaccaatgatggcaactgtctgtgtgcagcagctattgaagactctctttgggacaaacttagcctcacagtttgatggcttatcttccaaccccagcaagtctcagtgccgagctcagcgccttggctcttcaagtgtgaggcccggcttatatcactactgcttcatggcaccatacacgcacttcacacaggccttggctgacgcaagcctgaggaacatggtgcaggcggagcaggagcgtgatgcctcggggtggtttgatgtactccagaaagtgtctgcccaattgaagacgaacctaacaagcgtcacaaagaaccgtgcagataagaatgctattcataatcacattaggttatttgagcctcttgttataaaagcattgaagcagtacaccacgacaacatctgtacaattgcagaagcaggttttggatttgctggcacagctggttcagctacgggtcaattactgtctactggattcagaccaggtgttcatcgggtttgtgctgaagcagtttgagtacattgaagtgggccagttcagggaatcagaggcaattattccaaatatatttttcttcctggtattactgtcttatgagcgctaccattcaaaacagatcattggaattcctaaaatcatccagctgtgtgatggcatcatggccagtggaaggaaggccgttacacatgctatacctgctctgcagcccattgtccatgacctctttgtgttacgaggaacaaataaagctgatgcagggaaagagcttgagacacagaaggaggtggtggtctccatgctgttacgactcatccagtaccatcaggtgctggagatgttcatccttgtcctgcagcagtgccacaaggagaatgaggacaagtggaaacggctctctcggcaggtcgcagacatcatcctgcccatgttggccaagcagcagatgcatattgactctcatgaagcccttggagtgttaaataccttgtttgagattttggctccttcctccctacgtcctgtggacatgcttttgcggagtatgttcatcactccaagcacaatggcatctgtaagcactgtgcagctgtggatatctggaatcctcgccattctgagggttctcatttcccagtcaaccgaggacattgttctttgtcgtattcaggagctctccttctctccacacttgctctcctgtccagtgattaacaggttaaggggtggaggcggtaatgtaacactaggagaatgcagcgaagggaaacaaaagagtttgccagaagatacattctcaaggtttcttttacagctggttggtattcttctagaagacatcgttacaaaacagctcaaagtggacatgagtgaacagcagcatacgttctactgccaagagctaggcacactgctcatgtgtctgatccacatattcaaatctggaatgttccggagaatcacagcagctgccactagactcttcaccagtgatggctgtgaaggcagcttctatactctagagagcctgaatgcacgggtccgatccatggtgcccacgcacccagccctggtactgctctggtgtcagatcctacttctcatcaaccacactgaccaccggtggtgggcagaggtgcagcagacacccaagagacacagtctgtcctgcacgaagtcacttaacccccagaagtctggcgaagaggaggattctggctcggcagctcagctgggaatgtgcaatagagaaatagtgcgaagaggggcccttattctcttctgtgattatgtctgtcagaatctccatgactcagaacacttaacatggctcattgtgaatcacattcaagatctgatcagcttgtctcatgagcctccagtacaagactttattagtgccattcatcgtaattctgcagctagtggtctttttatccaggcaattcagtctcgctgtgaaaatctttcaacgccaaccactctgaagaaaacacttcagtgcttggaaggcatccatctcagccagtctggtgctgtgctcacactatatgtggacaggctcctgggcacccccttccgtgcgctggctcgcatggtcgacaccctggcctgtcgccgggtagaaatgcttttggctgcaaatttacagagcagcatggcccagttgccagaggaggaactaaacagaatccaagaacacctccagaacagtgggcttgcacaaagacaccaaaggctctattcactgctggacagattccgactctctactgtgcaggactcacttagccccttgcccccagtcacttcccacccactggatggggatgggcacacatctctggaaacagtgagtccagacaaagactggtacctccagcttgtcagatcccagtgttggaccagatcagattctgcactgctggaaggtgcagagctggtcaaccgtatccctgctgaagatatgaatgacttcatgatgagctcggagttcaacctaagccttttggctccctgtttaagccttggcatgagcgagattgctaatggccaaaagagtcccctctttgaagcagcccgtggggtgattctgaaccgggtgaccagtgttgttcagcagcttcctgctgtccatcaagtcttccagcccttcctgcctatagagcccacggcctactggaacaagttgaatgatctgcttggtgataccacatcataccagtctctgaccatacttgcccgtgccctggcacagtacctggtggtgctctccaaagtgcctgctcatttgcaccttcctcctgagaaggagggggacacggtgaagtttgtggtaatgacagttgaggccctgtcatggcatttgatccatgagcagatcccactgagtctggacctccaagccgggctagactgctgctgcctggcactacaggtgcctggcctctggggggtgctgtcctccccagagtacgtgactcatgcctgctccctcatccattgtgtgcgattcatcctggaagccattgcagtacaacctggagaccagcttctcggtcctgaaagcaggtcacatactccaagagctgtcagaaaggaggaagtagactcagatatacaaaacctcagtcatgtcacttcggcctgcgagatggtggcagacatggtggaatccctgcagtcagtgctggccttgggccacaagaggaacagcaccctgccttcatttctcacagctgtgctgaagaacattgttatcagtctggcccgactccccctagttaacagctatactcgtgtgcctcctctggtatggaaactcgggtggtcacccaagcctggaggggattttggcacagtgtttcctgagatccctgtagagttcctccaggagaaggagatcctcaaggagttcatctaccgcatcaacaccctagggtggaccaatcgtacccagttcgaagaaacttgggccaccctccttggtgtcctggtgactcagcccctggtgatggaacaggaagagagcccaccagaggaagacacagaaagaacccagatccatgtcctggctgtgcaggccatcacctctctagtgctcagtgcaatgaccgtgcctgtggctggcaatccagctgtaagctgcttggagcaacagccccggaacaagccactgaaggctctcgataccagatttggaagaaagctgagcatgatcagagggattgtagaacaagaaatccaagagatggtttcccagagagagaatactgccactcaccattctcaccaggcgtgggatcctgtcccttctctgttaccagctactacaggtgctcttatcagccatgacaagctgctgctgcagatcaacccagagcgggagccaggcaacatgagctacaagctgggccaggtgtccatacactccgtgtggctgggaaataacatcacacccctgagagaggaggaatgggatgaggaagaagaggaagaaagtgatgtccctgcaccaacgtcaccacctgtgtctccagtcaattccagaaaacaccgtgccggggttgatattcactcctgttcgcagtttctgcttgaattgtacagccgatggatcctgccatccagtgcagccagaaggacccccgtcatcctgatcagtgaagtggttcgatctcttcttgtagtgtcagacttattcaccgaacgtacccagtttgaaatgatgtatctgacgctgacagaactacggagagtgcacccttcagaagatgagatcctcattcagtacctggtgcctgccacctgtaaggcagctgctgtccttggaatggacaaaactgtggcagagccagtcagccgcctactggagagcacactgaggagcagccacctgcccagccagatcggagccctgcacggcatcctctatgtgttggagtgtgacctcttggatgacactgcaaagcagctcattccagttgttagtgactatctgctgtccaacctcaaaggaatagcccactgcgtgaacattcacagccagcagcatgtgctggtaatgtgtgccactgctttctacctgatggaaaactaccctctggatgtgggaccagaattttcagcatctgtgatacagatgtgtggagtaatgctgtctggaagtgaggagtccaccccctccatcatttaccactgtgccctccggggtctggagcggctcctgctgtctgagcagctatctcggctagacacagagtccttggtcaagctaagtgtggacagagtgaatgtacaaagcccacacagggccatggcagccctaggcctgatgctcacctgcatgtacacaggaaaggaaaaagccagtccaggcagagcttctgaccccagccctgctacacctgacagcgagtctgtgattgtagctatggagcgagtgtctgttctctttgataggatccgcaagggatttccctgtgaagccagggttgtggcaaggatcctgcctcagttcctagatgacttctttccacctcaagatgtcatgaacaaagtcattggagagttcctgtccaatcagcagccatacccacagttcatggccactgtagtttacaaggtttttcagactctgcacagtgctgggcagtcatccatggtccgggactgggtcatgctgtccctgtccaacttcacacaaagaactccagttgccatggccatgtggagcctctcctgcttccttgttagcgcatctaccagcccatgggtttctgcgatccttccacatgtcatcagcaggatgggcaaactggaacaggtggatgtgaaccttttctgcctggttgccacagacttctacagacaccagatagaggaggaattcgaccgcagggctttccagtctgtgtttgaggtggtggctgcaccaggaagtccataccacaggctgcttgcttgtttgcaaaatgttcacaaggtcaccacctgctgagtagtgcctgtgggacaaaaggctgaaagaaggcagctgctggggcctgagcctccaggagcctgctccaagcttctgctggggctgccttggccgtgcaggcttccacttgtgtcaagtggacagccaggcaatggcaggagtgctttgcaatgagggctatgcagggaacatgcactatgttggggttgagcctgagtcctgggtcctggcctcgctgcagctggtgacagtgctaggttgaccaggtgtttgtctttttcctagtgttcccctggccatagtcgccaggttgcagctgccctggtatgtggatcagaagtcctagctcttgccagatggttctgagcccgcctgctccactgggctggagagctccctcccacatttacccagtaggcatacctgccacaccagtgtctggacacaaaatgaatggtgtgtggggctgggaactggggctgccaggtgtccagcaccattttcctttctgtgttttcttctcaggagttaaaatttaattatatcagtaaagagattaattttaatgt

SEQ ID NO: 287 - DNA, 20 bp, synthetic construct
  source 1..20: mol_type = other DNA
attacagtctcaccacgccc

SEQ ID NO: 288 - DNA, 20 bp, synthetic construct
  source 1..20: mol_type = other DNA
gacaagggaagacccaagtg

(19) United States
(12) Patent Application Publication
(10) Pub. No.: US 2025/0223589 A1
(43) Pub. Date: Jul. 10, 2025

(54) Selective Reduction of Allelic Variants

(71) Applicant: Ionis Pharmaceuticals, Inc., Carlsbad, CA (US)

(72) Inventors: C. Frank Bennett, Carlsbad, CA (US); Susan M. Freier, San Diego, CA (US); Sarah Greenlee, San Diego, CA (US); Eric E. Swayze, Encinitas, CA (US)

(73) Assignee: Ionis Pharmaceuticals, Inc., Carlsbad, CA (US)

(21) Appl. No.: 18/811,243

(22) Filed: Aug. 21, 2024

Related U.S. Application Data
(63) Continuation of application No. 17/577,832, filed on Jan. 18, 2022, now Pat. No. 12,110,491.
Application No. 17/577,832 is a continuation of application No. 15/961,567, filed on Apr. 24, 2018, now abandoned.
Application No. 15/961,567 is a continuation of application No. 14/581,235, filed on Dec. 23, 2014, now abandoned.
Application No. 14/581,235 is a continuation of application No. 13/577,616, filed on Oct. 4, 2012, now Pat. No. 8,957,040.
(60) Provisional application No. 61/371,635, filed on Aug. 6, 2010.
(60) Provisional application No. 61/302,469, filed on Feb. 8, 2010.

(51) Int. Cl. C12N 15/113

(52) U.S. Cl. CPC C12N 15/113; C12N 2310/11; C12N 2310/315; C12N 2310/321; C12N 2310/3231; C12N 2310/3341; C12N 2310/341; C12N 2310/346; C12N 2320/34; C12N 2320/35

(57) ABSTRACT

Disclosed herein are antisense compounds and methods for selectively reducing expression of an allelic variant of a gene containing a single nucleotide polymorphism (SNP). Such methods, compounds, and composition are useful to treat, prevent, or ameliorate diseases, including neurodegenerative diseases, such as Huntington's Disease (HD).

### SEQUENCE LISTING

The present application contains a Sequence Listing which has been submitted electronically in XML format. Said XML copy, created on Aug. 2, 2024, is named “BIOL0124SEQ.xml” and is 479,584 bytes in size. The information in the electronic format of the sequence listing is incorporated herein by reference in its entirety.

### FIELD OF THE INVENTION

Embodiments of the present invention provide methods, compounds, and compositions for selectively reducing expression of an allelic variant of a gene containing a single nucleotide polymorphism (SNP). Such methods, compounds, and compositions are useful to treat, prevent, or ameliorate diseases.

### BACKGROUND OF THE INVENTION

Genetic diseases are caused by abnormalities in genes or chromosomes. Such abnormalities may include insertions, deletions, and expansions. Huntington's Disease (HD) is one example of a genetic disease caused by an expansion. HD is a progressive neurodegenerative disorder that is inherited in a dominant fashion and results from a mutation that expands the polymorphic trinucleotide (CAG) tract in the huntingtin gene (HTT). The average CAG tract size in the general population is 17-26 repeats (wild type allele), however, in HD patients the CAG tract has expanded to 36 repeats or more (mutant allele) (Huntington's Disease Collaborative Research Group 1993. Cell 72 (6): 971-83). The HTT gene encodes the HTT protein and the expanded CAG tract results in a pathological increase in the polyglutamine repeats near the N-terminal of the protein. Individuals carry two copies of the HTT gene and one mutant allele is sufficient to result in HD.

HTT protein appears to have a role during development of the nervous system and a protective role in cells. In mouse models, constitutive knockout of the HTT gene is lethal during embryonic development (Nasir et al 1995. Cell 81 (5): 811-23), while adult inactivation of the HTT gene leads to progressive cell death in the brain and the testes (Dragatsis et al 2000. Nat. Genet 26:300-306). Reduction of huntingtin expression from the wild type allele may, therefore, have negative consequences.

Like HD, there are disorders for which a strategy of selective reduction of a mutant allele would be beneficial. Thus, there remains an unmet need to selectively reduce expression of mutant allelic variants like that of HTT, which are causative of disease, over the wild type variant, which appears to be necessary for normal cellular processes.

### BRIEF DESCRIPTION OF THE DRAWINGS

FIG. 1A-EEEEEEE provides the mRNA (SEQ ID NO:2) and genomic (SEQ ID NO:1) HTT sequence showing SNP positions.

### SUMMARY OF THE INVENTION

Provided herein are methods, compounds, and compositions for selectively reducing expression of an allelic variant of a gene containing a single nucleotide polymorphism (SNP). Such methods, compounds, and compositions are useful to treat, prevent, or ameliorate diseases. SNPs may be associated with a mutant allele, the expression of which causes disease. In certain embodiments, the expressed gene product of a mutant allele results in aggregation of the mutant proteins causing disease. In certain embodiments, the expressed gene product of a mutant allele results in gain of function causing disease.

In certain embodiments, selective reduction of mRNA and protein expression of a mutant allele is achieved by targeting a SNP located on the mutant allele with an antisense compound. In certain embodiments, the antisense compound is an antisense oligonucleotide

In certain embodiments, antisense compounds designed to selectively reduce an allelic variant of a gene containing a SNP are created based on potency and selectivity of the antisense compound as well as population genetics.

### DETAILED DESCRIPTION OF THE INVENTION

It is to be understood that both the foregoing general description and the following detailed description are exemplary and explanatory only and are not restrictive of the invention, as claimed. Herein, the use of the singular includes the plural unless specifically stated otherwise. As used herein, the use of “or” means “and/or” unless stated otherwise. Furthermore, the use of the term “including” as well as other forms, such as “includes” and “included”, is not limiting. Also, terms such as “element” or “component” encompass both elements and components comprising one unit and elements and components that comprise more than one subunit, unless specifically stated otherwise.

The section headings used herein are for organizational purposes only and are not to be construed as limiting the subject matter described. All documents, or portions of documents, cited in this application, including, but not limited to, patents, patent applications, articles, books, and treatises, are hereby expressly incorporated by reference for the portions of the document discussed herein, as well as in their entirety.

### Definitions

Unless specific definitions are provided, the nomenclature utilized in connection with, and the procedures and techniques of, analytical chemistry, synthetic organic chemistry, and medicinal and pharmaceutical chemistry described herein are those well known and commonly used in the art. Standard techniques may be used for chemical synthesis, and chemical analysis. Where permitted, all patents, applications, published applications and other publications, GENBANK Accession Numbers and associated sequence information obtainable through databases such as National Center for Biotechnology Information (NCBI) and other data referred to throughout in the disclosure herein are incorporated by reference for the portions of the document discussed herein, as well as in their entirety.

Unless otherwise indicated, the following terms have the following meanings:

“2′-O-methoxyethyl” (also 2′-MOE and 2′-O(CH₂)₂—OCH₃) refers to an O-methoxy-ethyl modification of the 2′ position of a furosyl ring. A 2′-O-methoxyethyl modified sugar is a modified sugar.

“2′-O-methoxyethyl nucleotide” means a nucleotide comprising a 2′-O-methoxyethyl modified sugar moiety.

“5-methylcytosine” means a cytosine modified with a methyl group attached to the 5′ position. A 5-methylcytosine is a modified nucleobase.

“Active pharmaceutical agent” means the substance or substances in a pharmaceutical composition that provide a therapeutic benefit when administered to an individual. For example, in certain embodiments an antisense oligonucleotide targeted to an allelic variant is an active pharmaceutical agent.

“Active target region” or “target region” means a region to which one or more active antisense compounds is targeted. “Active antisense compounds” means antisense compounds that reduce target nucleic acid levels or protein levels.

“Administered concomitantly” refers to the co-administration of two agents in any manner in which the pharmacological effects of both are manifest in the patient at the same time. Concomitant administration does not require that both agents be administered in a single pharmaceutical composition, in the same dosage form, or by the same route of administration. The effects of both agents need not manifest themselves at the same time. The effects need only be overlapping for a period of time and need not be coextensive.

“Administering” means providing a pharmaceutical agent to an individual, and includes, but is not limited to administering by a medical professional and self-administering.

“Allele” is one member of a pair of genes or one member of a series of different forms of a DNA sequences that can exist at a single locus or marker on a specific chromosome. For a diploid organism or cell or for autosomal chromosomes, each allelic pair will normally occupy corresponding positions (loci) on a pair of homologous chromosomes, one inherited from the mother and one inherited from the father. If these alleles are identical, the organism or cell is said to be ‘homozygous’ for that allele; if they differ, the organism or cell is said to be ‘heterozygous’ for that allele. “Major allele” refers to an allele containing the nucleotide present in a statistically significant proportion of individuals in the human population. “Minor allele” refers to an allele containing the nucleotide present in a relatively small proportion of individuals in the human population. “Wild type allele” refers to the genotype typically not associated with disease or dysfunction of the gene product. “Mutant allele” refers to the genotype associated with disease or dysfunction of the gene product.

“Allelic variant” refers to one of the pair of genes or DNA sequence existing at a single locus. For example, an allelic variant may refer to either the major allele or the minor allele.

“Amelioration” refers to a lessening of at least one indicator, sign, or symptom of an associated disease, disorder, or condition. The severity of indicators may be determined by subjective or objective measures, which are known to those skilled in the art.

“Animal” refers to a human or non-human animal, including, but not limited to, mice, rats, rabbits, dogs, cats, pigs, and non-human primates, including, but not limited to, monkeys and chimpanzees.

“Antibody” refers to a molecule characterized by reacting specifically with an antigen in some way, where the antibody and the antigen are each defined in terms of the other. Antibody may refer to a complete antibody molecule or any fragment or region thereof, such as the heavy chain, the light chain, Fab region, and Fc region.

“Antisense activity” means any detectable or measurable activity attributable to the hybridization of an antisense compound to its target nucleic acid. In certain embodiments, antisense activity is a decrease in the amount or expression of a target nucleic acid or protein encoded by such target nucleic acid.

“Antisense compound” means an oligomeric compound that is capable of undergoing hybridization to a target nucleic acid through hydrogen bonding.

“Antisense inhibition” means reduction of target nucleic acid levels or target protein levels in the presence of an antisense compound complementary to a target nucleic acid compared to target nucleic acid levels or target protein levels in the absence of the antisense compound.

“Antisense oligonucleotide” means a single-stranded oligonucleotide having a nucleobase sequence that permits hybridization to a corresponding region or segment of a target nucleic acid.

“Bicyclic sugar” means a furosyl ring modified by the bridging of two ring atoms. A bicyclic sugar is a modified sugar.

“Bicyclic nucleoside” means a nucleoside having a sugar moiety comprising a bridge connecting two carbon atoms of the sugar ring, thereby forming a bicyclic ring system. In certain embodiments, the bridge connects the 4′-carbon and the 2′-carbon of the sugar ring.

“Cap structure” or “terminal cap moiety” means chemical modifications, which have been incorporated at either terminus of an antisense compound.

“cEt” or “constrained ethyl” means a bicyclic nucleoside having a sugar moiety comprising a bridge connecting the 4′-carbon and the 2′-carbon, wherein the bridge has the formula: 4′-CH(CH₃)—O-2′.

“Chemically distinct region” refers to a region of an antisense compound that is in some way chemically different than another region of the same antisense compound. For example, a region having 2′-O-methoxyethyl nucleotides is chemically distinct from a region having nucleotides without 2′-O-methoxyethyl modifications.

“Chimeric antisense compound” means an antisense compound that has at least two chemically distinct regions.

“Co-administration” means administration of two or more pharmaceutical agents to an individual. The two or more pharmaceutical agents may be in a single pharmaceutical composition, or may be in separate pharmaceutical compositions. Each of the two or more pharmaceutical agents may be administered through the same or different routes of administration. Co-administration encompasses parallel or sequential administration.

“Complementarity” means the capacity for pairing between nucleobases of a first nucleic acid and a second nucleic acid.

“Contiguous nucleobases” means nucleobases immediately adjacent to each other.

“Differentiating polymorphism” means a variation in a nucleotide sequence that permits differentiation between a wild type and a mutant allele of a nucleic acid sequence. Differentiating polymorphisms may include insertions or deletions of one or a few nucleotides in a sequence, or changes in one or a few nucleotides in a sequence. A differentiating polymorphism or polymorphic allele can be in linkage disequilibrium with one or more other polymorphisms or polymorphic alleles.

“Diluent” means an ingredient in a composition that lacks pharmacological activity, but is pharmaceutically necessary or desirable. For example, the diluent in an injected composition may be a liquid, e.g. saline solution.

“Dose” means a specified quantity of a pharmaceutical agent provided in a single administration, or in a specified time period. In certain embodiments, a dose may be administered in one, two, or more boluses, tablets, or injections. For example, in certain embodiments where subcutaneous administration is desired, the desired dose requires a volume not easily accommodated by a single injection, therefore, two or more injections may be used to achieve the desired dose. In certain embodiments, the pharmaceutical agent is administered by infusion over an extended period of time or continuously. Doses may be stated as the amount of pharmaceutical agent per hour, day, week, or month.

“Effective amount” means the amount of active pharmaceutical agent sufficient to effectuate a desired physiological outcome in an individual in need of the agent. The effective amount may vary among individuals depending on the health and physical condition of the individual to be treated, the taxonomic group of the individuals to be treated, the formulation of the composition, assessment of the individual's medical condition, and other relevant factors.

“Fully complementary” or “100% complementary” means each nucleobase of a first nucleic acid has a complementary nucleobase in a second nucleic acid. In certain embodiments, a first nucleic acid is an antisense compound and a target nucleic acid is a second nucleic acid.

“Gapmer” means a chimeric antisense compound in which an internal region having a plurality of nucleosides that support RNase H cleavage is positioned between external regions having one or more nucleosides, wherein the nucleosides comprising the internal region are chemically distinct from the nucleoside or nucleosides comprising the external regions. The internal region may be referred to as the “gap” and the external regions may be referred to as the “wings.”

“Gap-widened” means a chimeric antisense compound having a gap segment of 12 or more contiguous 2′-deoxyribonucleosides positioned between and immediately adjacent to 5′ and 3′ wing segments having from one to six nucleosides.

“Gene product” refers to a biochemical material, such as RNA or protein, resulting from expression of a gene.

“Haplotype” means a set of alleles of closely linked loci on a chromosome that are generally inherited together. For example, a polymorphic allele at a first site in a nucleic acid sequence on the chromosome may be found to be associated with another polymorphic allele at a second site on the same chromosome, at a frequency other than would be expected for a random associate (e.g. “linkage equilibrium”). These two polymorphic alleles may be described as being in “linkage disequilibrium.” A haplotype may comprise two, three, four, or more alleles. The set of alleles in a haplotype along a given segment of a chromosome are generally transmitted to progeny together unless there has been a recombination event.

“High-affinity sugar modification” is a modified sugar moiety which when it is included in a nucleoside and said nucleoside is incorporated into an antisense oligonucleotide, the stability (as measured by Tm) of said antisense oligonucleotide: RNA duplex is increased as compared to the stability of a DNA:RNA duplex.

“High-affinity sugar-modified nucleoside” is a nucleoside comprising a modified sugar moiety that when said nucleoside is incorporated into an antisense compound, the binding affinity (as measured by Tm) of said antisense compound toward a complementary RNA molecule is increased. In certain embodiments of the invention at least one of said sugar-modified high-affinity nucleosides confers a ΔTm of at least 1 to 4 degrees per nucleoside against a complementary RNA as determined in accordance with the methodology described in Freier et al., Nucleic Acids Res., 1997, 25, 4429-4443, which is incorporated by reference in its entirety. In another aspect, at least one of the high-affinity sugar modifications confers about 2 or more, 3 or more, or 4 or more degrees per modification. In the context of the present invention, examples of sugar-modified high affinity nucleosides include, but are not limited to, (i) certain 2′-modified nucleosides, including 2′-substituted and 4′ to 2′ bicyclic nucleosides, and (ii) certain other non-ribofuranosyl nucleosides which provide a per modification increase in binding affinity such as modified tetrahydropyran and tricycloDNA nucleosides. For other modifications that are sugar-modified high-affinity nucleosides see Freier et al., Nucleic Acids Res., 1997, 25, 4429-4443.

“Hybridization” means the annealing of complementary nucleic acid molecules. In certain embodiments, complementary nucleic acid molecules include an antisense compound and a target nucleic acid.

“Immediately adjacent” means there are no intervening elements between the immediately adjacent elements.

“Individual” means a human or non-human animal selected for treatment or therapy.

“Internucleoside linkage” refers to the chemical bond between nucleosides.

“Linked nucleosides” means adjacent nucleosides which are bonded together.

“Mismatch” or “non-complementary nucleobase” refers to the case when a nucleobase of a first nucleic acid is not capable of pairing with the corresponding nucleobase of a second or target nucleic acid.

“Modified internucleoside linkage” refers to a substitution or any change from a naturally occurring internucleoside bond (i.e. a phosphodiester internucleoside bond).

“Modified nucleobase” refers to any nucleobase other than adenine, cytosine, guanine, thymidine, or uracil. An “unmodified nucleobase” means the purine bases adenine (A) and guanine (G), and the pyrimidine bases thymine (T), cytosine (C), and uracil (U).

“Modified nucleotide” means a nucleotide having, independently, a modified sugar moiety, modified internucleoside linkage, or modified nucleobase. A “modified nucleoside” means a nucleoside having, independently, a modified sugar moiety or modified nucleobase.

“Modified oligonucleotide” means an oligonucleotide comprising a modified internucleoside linkage, a modified sugar, or a modified nucleobase.

“Modified sugar” refers to a substitution or change from a natural sugar.

“Motif” means the pattern of chemically distinct regions in an antisense compound.

“Naturally occurring internucleoside linkage” means a 3′ to 5′ phosphodiester linkage.

“Natural sugar moiety” means a sugar found in DNA (2′-H) or RNA (2′-OH).

“Nuclease resistant modification” means a sugar modification or modified internucleoside linkage which, when incorporated into an oligonucleotide, makes said oligonucleotide more stable to degradation under cellular nucleases (e.g. exo- or endo-nucleases). Examples of nuclease resistant modifications include, but are not limited to, phosphorothioate internucleoside linkages, bicyclic sugar modifications, 2′-modified nucleotides, or neutral internucleoside linkages.

“Nucleic acid” refers to molecules composed of monomeric nucleotides. A nucleic acid includes ribonucleic acids (RNA), deoxyribonucleic acids (DNA), single-stranded nucleic acids, double-stranded nucleic acids, small interfering ribonucleic acids (siRNA), and microRNAs (miRNA).

“Nucleobase” means a heterocyclic moiety capable of pairing with a base of another nucleic acid.

“Nucleobase sequence” means the order of contiguous nucleobases independent of any sugar, linkage, or nucleobase modification.

“Nucleoside” means a nucleobase linked to a sugar.

“Nucleoside mimetic” includes those structures used to replace the sugar or the sugar and the base and not necessarily the linkage at one or more positions of an oligomeric compound such as for example nucleoside mimetics having morpholino, cyclohexenyl, cyclohexyl, tetrahydropyranyl, bicyclo or tricyclo sugar mimetics e.g. non furanose sugar units. Nucleotide mimetic includes those structures used to replace the nucleoside and the linkage at one or more positions of an oligomeric compound such as for example peptide nucleic acids or morpholinos (morpholinos linked by —N(H)—C(—O)—O— or other non-phosphodiester linkage). Sugar surrogate overlaps with the slightly broader term nucleoside mimetic but is intended to indicate replacement of the sugar unit (furanose ring) only. The tetrahydropyranyl rings provided herein are illustrative of an example of a sugar surrogate wherein the furanose sugar group has been replaced with a tetrahydropyranyl ring system.

“Nucleotide” means a nucleoside having a phosphate group covalently linked to the sugar portion of the nucleoside.

“Oligomeric compound” or “oligomer” means a polymer of linked monomeric subunits which is capable of hybridizing to at least a region of a nucleic acid molecule.

“Oligonucleotide” means a polymer of linked nucleosides each of which can be modified or unmodified, independent one from another.

“Parenteral administration” means administration through injection or infusion. Parenteral administration includes subcutaneous administration, intravenous administration, intramuscular administration, intraarterial administration, intraperitoneal administration, or intracranial administration, e.g. intrathecal or intracerebroventricular administration.

“Peptide” means a molecule formed by linking at least two amino acids by amide bonds. Peptide refers to polypeptides and proteins.

“Pharmaceutical composition” means a mixture of substances suitable for administering to an individual. For example, a pharmaceutical composition may comprise one or more active pharmaceutical agents and a sterile aqueous solution.

“Pharmaceutically acceptable salts” means physiologically and pharmaceutically acceptable salts of antisense compounds, i.e., salts that retain the desired biological activity of the parent oligonucleotide and do not impart undesired toxicological effects thereto.

“Phosphorothioate linkage” means a linkage between nucleosides where the phosphodiester bond is modified by replacing one of the non-bridging oxygen atoms with a sulfur atom. A phosphorothioate linkage (P═S) is a modified internucleoside linkage.

“Portion” means a defined number of contiguous (i.e. linked) nucleobases of a nucleic acid. In certain embodiments, a portion is a defined number of contiguous nucleobases of a target nucleic acid. In certain embodiments, a portion is a defined number of contiguous nucleobases of an antisense compound.

“Prevent” refers to delaying or forestalling the onset or development of a disease, disorder, or condition for a period of time from minutes to indefinitely. Prevent also means reducing risk of developing a disease, disorder, or condition.

“Prodrug” means a therapeutic agent that is prepared in an inactive form that is converted to an active form within the body or cells thereof by the action of endogenous enzymes or other chemicals or conditions.

“Selectively reducing expression of an allelic variant” means reducing expression of one allele more than the other, differing allele among a set of alleles. For example, a mutant allele containing a single nucleotide polymorphism (SNP) may be reduced more than a wild type allele not containing the SNP.

“Side effects” means physiological responses attributable to a treatment other than the desired effects. In certain embodiments, side effects include injection site reactions, liver function test abnormalities, renal function abnormalities, liver toxicity, renal toxicity, central nervous system abnormalities, myopathies, and malaise. For example, increased aminotransferase levels in serum may indicate liver toxicity or liver function abnormality. For example, increased bilirubin may indicate liver toxicity or liver function abnormality.

“Single nucleotide polymorphism” or “SNP” means a single nucleotide variation between the genomes of individuals of the same species. In some cases, a SNP may be a single nucleotide deletion or insertion. In general, SNPs occur relatively frequently in genomes and thus contribute to genetic diversity. SNPs are thought to be mutationally more stable than other polymorphisms, lending their use in association studies in which linkage disequilibrium between markers and an unknown variant is used to map disease-causing mutations. The location of a SNP is generally flanked by highly conserved sequences. An individual may be homozygous or heterozygous for an allele at each SNP site. A heterozygous SNP allele can be a differentiating polymorphism. A SNP may be targeted with an antisense oligonucleotide, meaning that the SNP anneals to (or aligns with) position 1, 2, 3, 4, 5, 6, 7, 8, 9, 10, 11, 12, 13, 14, 15, 16, 17, 18, 19, or 20 of the antisense oligonucleotide. The remainder of the antisense oligonucleotide bases must have sufficient complementarity to the SNP site to facilitate hybridization.

“Single nucleotide polymorphism position” or “SNP position” refers to the nucleotide position of the SNP on a reference sequence.

“Single nucleotide polymorphism site” or “SNP site” refers to the nucleotides surrounding a SNP contained in a target nucleic acid to which an antisense compound is targeted.

“Single-stranded oligonucleotide” means an oligonucleotide which is not hybridized to a complementary strand.

“Specifically hybridizable” refers to an antisense compound having a sufficient degree of complementarity between an antisense oligonucleotide and a target nucleic acid to induce a desired effect, while exhibiting minimal or no effects on non-target nucleic acids under conditions in which specific binding is desired, i.e. under physiological conditions in the case of in vivo assays and therapeutic treatments.

“Targeting” or “targeted” means the process of design and selection of an antisense compound that will specifically hybridize to a target nucleic acid and induce a desired effect.

“Target nucleic acid,” “target RNA,” and “target RNA transcript” all refer to a nucleic acid capable of being targeted by antisense compounds.

“Target segment” means the sequence of nucleotides of a target nucleic acid to which an antisense compound is targeted. For example, for the purposes of this patent application, the target segment may be within the SNP site. “5′ target site” refers to the 5′-most nucleotide of a target segment. “3′ target site” refers to the 3′-most nucleotide of a target segment.

“Therapeutically effective amount” means an amount of a pharmaceutical agent that provides a therapeutic benefit to an individual.

“Treat” refers to administering a pharmaceutical composition to effect an alteration or improvement of a disease, disorder, or condition.

“Unmodified nucleotide” means a nucleotide composed of naturally occurring nucleobases, sugar moieties, and internucleoside linkages. In certain embodiments, an unmodified nucleotide is an RNA nucleotide (i.e. β-D-ribonucleosides) or a DNA nucleotide (i.e. β-D-deoxyribonucleoside).

### CERTAIN EMBODIMENTS

Embodiments of the present invention provide methods, compounds, and compositions for selectively inhibiting mRNA and protein expression of an allelic variant of a gene or DNA sequence. In certain embodiments, the allelic variant contains a single nucleotide polymorphism (SNP). In certain embodiments, the SNP is a differentiating polymorphism. In certain embodiments, a SNP is associated with a mutant allele. In certain embodiments, a SNP is in linkage disequilibrium with another polymorphism that is associated with or is causative of disease. In certain embodiments, a mutant allele is associated with disease. In certain embodiments, mRNA and protein expression of a mutant allele is associated with disease.

In certain embodiments, the expressed gene product of a mutant allele results in aggregation of the mutant proteins causing disease. In certain embodiments, the expressed gene product of a mutant allele results in gain of function causing disease. In certain embodiments, genes with an autosomal dominant mutation resulting in a toxic gain of function of the protein are the APP gene encoding amyloid precursor protein involved in Alzheimer's disease (Gene, 371:68, 2006); the PrP gene encoding prion protein involved in Creutzfeldt-Jakob disease and in fatal familial insomnia (Nat. Med. 1997, 3:1009); GFAP gene encoding glial fibrillary acidic protein involved in Alexander disease (J. Neurosci. 2006, 26:111623); alpha-synuclein gene encoding alpha-synuclein protein involved in Parkinson's disease (J. Clin. Invest. 2003, 111:145); SOD-1 gene encoding the SOD-1 protein involved in amyotrophic lateral sclerosis (Science 1998, 281:1851); atrophin-1 gene encoding atrophin-1 protein involved in dentato-rubral and pallido-luysian atrophy (DRPA) (Trends Mol. Med. 2001, 7:479); SCA1 gene encoding ataxin-1 protein involved in spino-cerebellar ataxia-1 (SCA1) (Protein Sci. 2003, 12:953); PLP gene encoding proteolipid protein involved in Pelizaeus-Merzbacher disease (NeuroMol Med. 2007, 4:73); DYTI gene encoding torsinA protein involved in Torsion dystonia (Brain Res. 2000, 877:379); and alpha-B crystalline gene encoding alpha-B crystalline protein involved in protein aggregation diseases, including cardiomyopathy (Cell 2007, 130: 427); alpha1-antitrypsin gene encoding alpha1-antitrypsin protein involved in chronic obstructive pulmonary disease (COPD), liver disease and hepatocellular carcinoma (New Engl J Med. 2002, 346:45); Ltk gene encoding leukocyte tyrosine kinase protein involved in systemic lupus erythematosus (Hum. Mol. Gen. 2004, 13:171); PCSK9 gene encoding PCSK9 protein involved in hypercholesterolemia (Hum Mutat. 2009, 30:520); prolactin receptor gene encoding prolactin receptor protein involved in breast tumors (Proc. Natl. Assoc. Sci. 2008, 105:4533); CCL5 gene encoding the chemokine CCL5 involved in COPD and asthma (Eur. Respir. J. 2008, 32:327); PTPN22 gene encoding PTPN22 protein involved in Type 1 diabetes, Rheumatoid arthritis, Graves disease, and SLE (Proc. Natl. Assoc. Sci. 2007, 104:19767); androgen receptor gene encoding the androgen receptor protein involved in spinal and bulbar muscular atrophy or Kennedy's disease (J Steroid Biochem. Mol. Biol. 2008, 108:245); CHMP4B gene encoding chromatin modifying protein-4B involved in progressive childhood posterior subcapsular cataracts (Am. J. Hum. Genet 2007, 81:596); FXR/NR1H4 gene encoding Farnesoid X receptor protein involved in cholesterol gallstone disease, arthrosclerosis and diabetes (Mol. Endocrinol. 2007, 21:1769); ABCA1 gene encoding ABCA1 protein involved in cardiovascular disease (Transl. Res. 2007, 149:205); CaSR gene encoding the calcium sensing receptor protein involved in primary hypercalciuria (Kidney Int. 2007, 71:1155); alpha-globin gene encoding alpha-globin protein involved in alpha-thallasemia (Science 2006, 312:1215); httlpr gene encoding HTTLPR protein involved in obsessive compulsive disorder (Am. J. Hum. Genet. 2006, 78:815); AVP gene encoding arginine vasopressin protein in stress-related disorders such as anxiety disorders and comorbid depression (CNS Neurol. Disord. Drug Targets 2006, 5:167); GNAS gene encoding G proteins involved in congenital visual defects, hypertension, metabolic syndrome (Trends Pharmacol. Sci. 2006, 27:260); APAF1 gene encoding APAF1 protein involved in a predisposition to major depression (Mol. Psychiatry 2006, 11:76); TGF-beta1 gene encoding TGF-beta1 protein involved in breast cancer and prostate cancer (Cancer Epidemiol. Biomarkers Prev. 2004, 13:759); AChR gene encoding acetylcholine receptor involved in congential myasthenic syndrome (Neurology 2004, 62:1090); P2Y12 gene encoding adenosine diphosphate (ADP) receptor protein involved in risk of peripheral arterial disease (Circulation 2003, 108:2971); LQT1 gene encoding LQT1 protein involved in atrial fibrillation (Cardiology 2003, 100:109); RET protooncogene encoding RET protein involved in sporadic pheochromocytoma (J. Clin. Endocrinol. Metab. 2003, 88:4911); filamin A gene encoding filamin A protein involved in various congenital malformations (Nat. Genet. 2003, 33:487); TARDBP gene encoding TDP-43 protein involved in amyotrophic lateral sclerosis (Hum. Mol. Gene.t 2010, 19:671); SCA3 gene encoding ataxin-3 protein involved in Machado-Joseph disease (PLOS One 2008, 3: e3341); SCA7 gene encoding ataxin-7 protein involved in spino-cerebellar ataxia-7 (PLOS One 2009, 4: e7232); and HTT gene encoding huntingtin protein involved in Huntington's disease (Neurobiol Dis. 1996, 3:183); and the CA4 gene encoding carbonic anhydrase 4 protein, CRX gene encoding cone-rod homeobox transcription factor protein, FSCN2 gene encoding retinal fascin homolog 2 protein, IMPDH1 gene encoding inosine monophosphate dehydrogenase 1 protein, NR2E3 gene encoding nuclear receptor subfamily 2 group E3 protein, NRL gene encoding neural retina leucine zipper protein, PRPF3 (RP18) gene encoding pre-mRNA splicing factor 3 protein, PRPF8 (RP13) gene encoding pre-mRNA splicing factor 8 protein, PRPF31 (RP11) gene encoding pre-mRNA splicing factor 31 protein, RDS gene encoding peripherin 2 protein, ROM1 gene encoding rod outer membrane protein 1 protein, RHO gene encoding rhodopsin protein, RP1 gene encoding RP1 protein, RPGR gene encoding retinitis pigmentosa GTPase regulator protein, all of which are involved in Autosomal Dominant Retinitis Pigmentosa disease (Adv Exp Med Biol. 2008, 613:203)

In certain embodiments, selective reduction of mRNA and protein expression of a mutant allele is achieved by targeting a SNP located on the mutant allele with an antisense compound. In certain embodiments, the antisense compound is an antisense oligonucleotide. In certain embodiments, the antisense compound is not a ribozyme, a double stranded siRNA, or an shRNA. In certain embodiments, the antisense oligonucleotide may have one or more modified sugar(s), nucleobase(s), or internucleoside linkage(s). In certain embodiments, the antisense oligonucleotide is complementary to the SNP site. In certain embodiments, the antisense oligonucleotide is at least 65%, 70%, 75%, 80%, 85%, 86%, 87%, 88%, 89%, 90%, 91%, 92%, 93%, 94%, 95%, 96%, 97%, 98%, or 99% complementary to the SNP site. In certain embodiments, the antisense oligonucleotide is 100% complementary to the SNP site. In certain embodiments, the SNP site is 12, 13, 14, 15, 16, 17, 18, 19, 20, 21, 22, 23, 24, 25, 26, 27, 28, 29, or 30 nucleobases in length. In certain embodiments, the SNP anneals to position 1, 2, 3, 4, 5, 6, 7, 8, 9, 10, 11, 12, 13, 14, 15, 16, 17, 18, 19, or 20 of the antisense oligonucleotide.

In certain embodiments, antisense compounds designed to selectively reduce an allelic variant of a gene containing a SNP are created based on potency and selectivity of the antisense compound as well as population genetics.

In certain embodiments, selective reduction of mRNA and protein expression of an allelic variant of a gene containing a SNP occurs in a cell or tissue. In certain embodiments, the cell or tissue is in an animal. In certain embodiments, the animal is a human.

In certain embodiments, described herein are compounds comprising a modified antisense oligonucleotide consisting of 12 to 30 linked nucleosides targeted to a single nucleotide polymorphism site, wherein the modified oligonucleotide comprises a wing-gap-wing motif with a 5′ wing region positioned at the 5′ end of a deoxynucleoside gap, and a 3′ wing region positioned at the 3′ end of the deoxynucleoside gap, wherein position 5, 6, 7, 8, 9, 10, 11, 12, 13, 14, or 15 of the modified oligonucleotide, as counted from the 5′ terminus of the modified oligonucleotide, or positions 1, 2, 3, 4, 5, 6, 7, 8, or 9 of the modified oligonucleotide, as counted from the 5′ terminus of the gap, aligns with the single nucleotide polymorphism.

In certain embodiments, the single nucleotide polymorphism site is on a mutant allele that is associated with a disease. In certain embodiments, the single nucleotide polymorphism site contains a differentiating polymorphism.

In certain embodiments, the modified antisense oligonucleotide consists of 12 to 20 linked nucleosides. In certain embodiments, modified antisense oligonucleotide consists of 15 to 20 linked nucleosides. In certain embodiments, the modified antisense oligonucleotide consists of 15 to 19 linked nucleosides.

In certain embodiments, position 8, 9, or 10 of the modified oligonucleotide, as counted from the 5′ terminus of the modified oligonucleotide, or positions 4, 5, or 6 of the modified oligonucleotide, as counted from the 5′ terminus of the gap, aligns with the single nucleotide polymorphism.

In certain embodiments, the gap region is 7-11 nucleosides in length, the 5′ wing region is 1-6 nucleobases in length and the 3′ wing region is 1-6 nucleobases in length.

In certain embodiments, the wing-gap-wing motif is any one of the group consisting of 5-10-5, 2-9-6, 3-9-3, 3-9-4, 3-9-5, 4-7-4, 4-9-3, 4-9-4, 4-9-5, 4-10-5, 4-11-4, 4-11-5, 5-7-5, 5-8-6, 5-9-3, 5-9-5, 5-10-4, 5-10-5, 6-7-6, 6-8-5, and 6-9-2. In certain embodiments, the wing-gap-wing motif is any one of the group consisting of 2-9-6, 4-9-5, and 4-11-4.

In certain embodiments, at least one internucleoside linkage is a modified internucleoside linkage. In certain embodiments, each internucleoside linkage is a phosphorothioate internucleoside linkage.

In certain embodiments, at least one nucleoside comprises a modified nucleobase. In certain embodiments, the modified nucleobase is a 5′-methylcytosine.

In certain embodiments, at least one nucleoside of at least one of the wing regions comprises a modified sugar or sugar surrogate. In certain embodiments, each of the nucleosides of each wing region comprises a modified sugar or sugar surrogate. In certain embodiments, the sugar or sugar surrogate is a 2′-O-methoxyethyl modified sugar.

In certain embodiments, at least one of the wing regions comprises a 4′ to 2′ bicyclic nucleoside and at least one of the remaining wing nucleosides is a non-bicyclic 2′-modified nucleoside.

In certain embodiments, the non-bicyclic 2′-modified nucleoside is a 2′-O-methoxyethyl nucleoside.

In certain embodiments, the 4′ to 2′ bicyclic nucleoside is 4′-CH(CH3)-O-2′ bicyclic nucleoside.

In certain embodiments, the modified antisense oligonucleotide consisting of 17 linked nucleosides and wherein position 9 of the modified oligonucleotide, as counted from the 5′ terminus of the modified oligonucleotide, aligns with the differentiating polymorphism. In certain embodiments, the wing-gap-wing motif is 2-9-6.

In certain embodiments, described herein are compounds comprising a modified oligonucleotide consisting of 18 linked nucleosides and 90% complementary to a differentiating polymorphism, wherein the modified oligonucleotide comprises a wing-gap-wing motif, wherein position 9 of the modified oligonucleotide, as counted from the 5′ terminus of the modified oligonucleotide, aligns with the differentiating polymorphism; wherein each nucleoside of each wing segment comprises a 2′-O-methoxyethyl sugar; and wherein the wing-gap-wing motif is 4-9-5.

In certain embodiments, described herein are compounds comprising a modified oligonucleotide consisting of 19 linked nucleosides and 90% complementary to a differentiating polymorphism, wherein the modified oligonucleotide comprises a wing-gap-wing motif, wherein position 10 of the modified oligonucleotide, as counted from the 5′ terminus of the modified oligonucleotide, aligns with the differentiating polymorphism; wherein each nucleoside of each wing segment comprises a 2′-O-methoxyethyl sugar; and wherein the wing-gap-wing motif is 4-11-4.

In certain embodiments, described herein are compounds comprising a modified oligonucleotide consisting of 15 to 19 linked nucleosides and fully complementary to a differentiating polymorphism, wherein the modified oligonucleotide comprises a wing-gap-wing motif, wherein position 6, 7, 8, 9, 10, 11, 12, 13, or 14 of the modified oligonucleotide, as counted from the 5′ terminus of the modified oligonucleotide, aligns with the differentiating polymorphism; and at least one high-affinity sugar modification. In certain embodiments, the modified oligonucleotide is 100% complementary to the single nucleotide polymorphism site.

In certain embodiments, at least one of the wing regions comprises a high-affinity sugar modification. In certain embodiments, the high-affinity sugar modification is a bicyclic sugar. In certain embodiments, the bicyclic sugar comprises a 4′-CH(CH3)-O-2′ bridge.

In certain embodiments, at least one of positions 2, 3, 6, 9, 10, 11, 13, or 14 of the modified oligonucleotide, as counted from the 5′ terminus of the modified oligonucleotide, comprises the at least one high-affinity sugar modification.

In certain embodiments, at least one of positions 2, 3, 13, and 14 of the modified oligonucleotide, as counted from the 5′ terminus of the modified oligonucleotide, comprises the at least one high-affinity sugar modification.

In certain embodiments, each of nucleoside positions 2, 3, 13, and 14 of the modified oligonucleotide, as counted from the 5′ terminus of the modified oligonucleotide, comprise the at least one high-affinity sugar modification.

In certain embodiments, the high-affinity sugar modification is a bicyclic sugar. In certain embodiments, the bicyclic sugar comprises a 4′-CH(CH3)-O-2′ bridge.

In certain embodiments, the wing-gap-wing motif is any of the group consisting of 3-9-3, 4-9-4, and 5-9-5.

In certain embodiments, described herein are compounds comprising a modified oligonucleotide consisting of 15, 17, or 19 linked nucleosides and fully complementary to a differentiating polymorphism, wherein the modified oligonucleotide comprises a wing-gap-wing motif, wherein position 6, 8, 10, or 14 of the modified oligonucleotide, as counted from the 5′ terminus of the modified oligonucleotide, aligns with the differentiating polymorphism; and at least one high-affinity sugar modification.

In certain embodiments, at least one of positions 2, 3, 6, 9, 10, 11, 13, or 14 of the modified oligonucleotide, as counted from the 5′ terminus of the modified oligonucleotide, comprises the at least one high-affinity sugar modification.

In certain embodiments, the high-affinity sugar modification is a bicyclic sugar. In certain embodiments, the bicyclic sugar comprises a 4′-CH(CH3)-O-2′ bridge.

In certain embodiments, the wing-gap-wing motif is any of the group consisting of 3-9-3, 4-9-4, and 5-95.

In certain embodiments, described herein are compounds comprising a modified oligonucleotide consisting of 15 linked nucleosides and 90% complementary to a differentiating polymorphism, wherein the modified oligonucleotide comprises a wing-gap-wing motif, wherein position 8 of the modified oligonucleotide, as counted from the 5′ terminus of the modified oligonucleotide, aligns with the differentiating polymorphism; and at least one high-affinity sugar modification. In certain embodiments, the modified oligonucleotide is 100% complementary to the differentiating polymorphism.

In certain embodiments, each of nucleoside positions 2, 3, 13, and 14 of the modified oligonucleotide, as counted from the 5′ terminus of the modified oligonucleotide, comprise the at least one high-affinity sugar modification.

In certain embodiments, the high-affinity sugar modification is a bicyclic sugar. In certain embodiments, the bicyclic sugar comprises a 4′-CH(CH3)-O-2′ bridge.

In certain embodiments, the wing-gap-wing motif is 3-9-3.

In certain embodiments, described herein are methods of selectively reducing expression of an allelic variant of a gene containing a single nucleotide polymorphism in a cell, tissue, or animal, comprising administering to the cell, tissue, or animal a compound comprising a modified oligonucleotide complementary to a differentiating polymorphism, wherein the modified oligonucleotide comprises a wing-gap-wing motif and wherein position 5, 6, 7, 8, 9, 10, 11, 12, 13, 14, or 15 of the modified oligonucleotide, as counted from the 5′ terminus of the modified oligonucleotide, aligns with the differentiating polymorphism. In certain embodiments, the modified oligonucleotide is 90% complementary to the single differentiating polymorphism. In certain embodiments, the modified oligonucleotide is 95% complementary to the single nucleotide polymorphism site. In certain embodiments, the modified oligonucleotide is 100% complementary to the single nucleotide polymorphism site.

In certain embodiments, the single nucleotide polymorphism site is from 12 to 30 nucleobases in length. In certain embodiments, the single nucleotide polymorphism site is from 15 to 25 nucleobases in length. In certain embodiments, the single nucleotide polymorphism site is from 17 to 22 nucleobases in length. In certain embodiments, the single nucleotide polymorphism site is 17 nucleobases in length. In certain embodiments, the single nucleotide polymorphism site is 18 nucleobases in length. In certain embodiments, the single nucleotide polymorphism site is 19 nucleobases in length. In certain embodiments, the single nucleotide polymorphism site is 20 nucleobases in length.

In certain embodiments, the allelic variant is associated with disease. In certain embodiments, the disease is Huntington's Disease.

In certain embodiments, the modified oligonucleotide is a single-stranded oligonucleotide.

In certain embodiments, at least one internucleoside linkage is a modified internucleoside linkage. In certain embodiments, each internucleoside linkage is a phosphorothioate internucleoside linkage.

In certain embodiments, at least one nucleoside comprises a modified nucleobase. In certain embodiments, the at least one modified nucleobase is a 5′-methylcytosine.

In certain embodiments, at least one nucleoside comprises a modified sugar. In certain embodiments, the modified sugar is a high-affinity sugar modification. In certain embodiments, the high-affinity sugar is a bicyclic sugar. In certain embodiments, each bicyclic sugar comprises a 4′-CH(CH₃)—O-2′ bridge.

In certain embodiments, at least one of nucleoside positions 2, 3, 13, and 14 of the modified oligonucleotide, counting from the 5′ terminus of the modified oligonucleotide, comprises a nucleoside having a bicyclic sugar wherein the bicyclic sugar comprises a 4′-CH(CH₃)—O-2′ bridge.

In certain embodiments, each of nucleoside positions 2, 3, 13, and 14 of the modified oligonucleotide, counting from the 5′ terminus of the modified oligonucleotide, comprises a bicyclic sugar wherein the bicyclic sugar comprises a 4′-CH(CH₃)—O-2′ bridge.

In certain embodiments, the at least one modified sugar comprises a 2′-O-methoxyethyl. In certain embodiments, each nucleoside positioned in a wing segment of the modified oligonucleotide comprises a 2′-O-methoxyethyl modification.

In certain embodiments, the wing-gap-wing motif is any of the group consisting of 2-9-6, 3-9-3, 3-9-4, 3-9-5, 4-7-4, 4-9-4, 4-9-5, 4-10-5, 4-11-4, 4-11-5, 5-7-5, 5-8-6, 5-9-3, 5-9-5, 5-10-4, 5-10-5, 6-7-6, 6-8-5, and 6-9-2.

In certain embodiments, the modified oligonucleotide is not a ribozyme, a double stranded siRNA, or an shRNA.

In certain embodiments, the single nucleotide polymorphism site is on a mutant allele that is associated with disease. In certain embodiments, the single nucleotide polymorphism site contains a differentiating polymorphism.

In certain embodiments, the modified antisense oligonucleotide consists of 12 to 20 linked nucleosides. In certain embodiments, the modified antisense oligonucleotide consists of 15 to 19 linked nucleosides.

In certain embodiments, the gap region is 7 to 11 nucleosides in length, the 5′ wing region is 1 to 6 nucleobases in length and 3′ wing region is 1 to 6 nucleobases in length.

In certain embodiments, wherein at least one nucleoside of at least one of the wing regions comprises a modified sugar or sugar surrogate.

In certain embodiments, each of the nucleosides of each wing region comprises a modified sugar or sugar surrogate. In certain embodiments, the sugar or sugar surrogate is a 2′-O-methoxyethyl modified sugar.

In certain embodiments, at least one of the wing regions comprises a 4′ to 2′ bicyclic nucleoside and at least one of the remaining wing nucleosides is a non-bicyclic 2′-modified nucleoside.

In certain embodiments, the non-bicyclic 2′-modified nucleoside is a 2′-O-methoxyethyl nucleoside.

In certain embodiments, 4′ to 2′ bicyclic nucleoside is a 4′-CH(CH3)-O-2′ bicyclic nucleoside.

In certain embodiments, described herein are methods of selectively reducing expression of an allelic variant of a gene containing a single nucleotide polymorphism in a cell, tissue, or animal, comprising administering to the cell, tissue, or animal a compound comprising a modified oligonucleotide complementary to a differentiating polymorphism, wherein the modified oligonucleotide comprises a wing-gap-wing motif and wherein position 5, 6, 7, 8, 9, 10, 11, 12, 13, 14, or 15 of the modified oligonucleotide, as counted from the 5′ terminus of the modified oligonucleotide, aligns with the differentiating polymorphism.

In certain embodiments, described herein are methods of selectively reducing expression of an allelic variant of a gene containing a single nucleotide polymorphism in a cell, tissue, or animal, comprising administering to the cell, tissue, or animal a compound comprising a modified oligonucleotide consisting of 12 to 30 linked nucleosides and complementary to a differentiating polymorphism, wherein the modified oligonucleotide comprises a wing-gap-wing motif and wherein position 5, 6, 7, 8, 9, 10, 11, 12, 13, 14, or 15 of the modified oligonucleotide, as counted from the 5′ terminus of the modified oligonucleotide aligns with the differentiating polymorphism; and wherein the allelic variant is a mutant allele.

In certain embodiments, the mutant allele is associated with any disease from the group consisting of Alzheimer's disease, Creutzfeldt-Jakob disease, fatal familial insomnia, Alexander disease, Parkinson's disease, amyotrophic lateral sclerosis, dentato-rubral and pallido-luysian atrophy DRPA, spino-cerebellar ataxia, Torsion dystonia, cardiomyopathy, chronic obstructive pulmonary disease (COPD), liver disease, hepatocellular carcinoma, systemic lupus erythematosus, hypercholesterolemia, breast cancer, asthma, Type 1 diabetes, Rheumatoid arthritis, Graves disease, SLE, spinal and bulbar muscular atrophy, Kennedy's disease, progressive childhood posterior subcapsular cataracts, cholesterol gallstone disease, arthrosclerosis, cardiovascular disease, primary hypercalciuria, alpha-thallasemia, obsessive compulsive disorder, Anxiety, comorbid depression, congenital visual defects, hypertension, metabolic syndrome, prostate cancer, congential myasthenic syndrome, peripheral arterial disease, atrial fibrillation, sporadic pheochromocytoma, congenital malformations, Machado-Joseph disease, Huntington's disease, and Autosomal Dominant Retinitis Pigmentosa disease.

In certain embodiments, described herein are methods of treating Huntington's Disease, comprising selectively reducing expression of an allelic variant of a gene containing a single nucleotide polymorphism in a cell, tissue, or animal, comprising administering to the cell, tissue, or animal a compound comprising a modified oligonucleotide consisting of 12 to 30 linked nucleosides and complementary to differentiating polymorphism, wherein the modified oligonucleotide comprises a wing-gap-wing motif and wherein position 5, 6, 7, 8, 9, 10, 11, 12, 13, 14, or 15 of the modified oligonucleotide, as counted from the 5′ terminus of the modified oligonucleotide, aligns with differentiating polymorphism; and wherein the allelic variant is associated with Huntington's Disease.

In certain embodiments, position 8, 9, or 10 of the modified oligonucleotide, as counted from eh 5′ terminus of the modified oligonucleotide, or positions 4, 5, or 6 of the modified oligonucleotide, as counted from the 5′ terminus of the gap, aligns with the single nucleotide polymorphism.

### Single Nucleotide Polymorphisms (SNPs)

Single-nucleotide polymorphisms (SNPs) are single base-pair alterations in the DNA sequence that represent a major source of genetic heterogeneity (Gene. 1999, 234:177). SNP genotyping is an important tool with which to investigate these genetic variants (Genome Res. 2000, 10:895; Trends Biotechnol. 2000, 18:77). In certain embodiments, antisense compounds designed to selectively reduce an allelic variant of a gene containing an SNP were selected based on potency, selectivity and population genetics coverage.

### Potency

In certain embodiments, antisense compounds designed to selectively reduce an allelic variant of a gene containing a SNP are created based on potency of the antisense compound. Potency generally refers to how amenable the targeted sequence area is to antisense inhibition. In certain embodiments, specific SNP sites may be particularly amenable to antisense inhibition. Certain such highly amenable SNP sites may be targeted by antisense compounds for selectively reducing an allelic variant of a gene. Potency is demonstrated by the percent inhibition of mutant mRNA achieved by the antisense oligonucleotides targeting a SNP compared to the percent inhibition of mutant mRNA achieved by the benchmark oligonucleotide.

### Selectivity

In certain embodiments, antisense compounds designed to selectively reduce an allelic variant of a gene containing a SNP are created based on selectivity of the antisense compound. Selectivity generally refers to antisense compounds comprising a particular sequence, motif, and chemical modification(s) that preferentially target the one or more differentiating polymorphisms (SNPs) in the RNA encoding a mutant HTT protein compared to the RNA encoding a wild type HTT protein. In certain embodiments, specific sequences, motifs, and chemical modification(s) are particularly selective in reducing an allelic variant of a gene containing a SNP. Certain such sequences, motifs, and chemical modification(s) are utilized to selectively reduce an allelic variant of a gene. Selectivity is demonstrated by the ability of the antisense oligonucleotide targeting a SNP to inhibit expression of the major allele or mutant allele preferentially compared to the minor allele or wild type allele.

### Population Genetics

In certain embodiments, antisense compounds designed to selectively reduce an allelic variant of a gene containing an SNP are created based on the population genetics of a population afflicted with disease. Population genetics means the frequency at which the SNP appears in the disease chromosome of patients afflicted with a particular disease. In certain embodiments, the disease is Huntington disease. Where potency and selectivity amongst antisense compounds is equal, SNP targets that have higher population genetics coverage are favored over SNPs that have a weaker association with disease chromosomes.

### Antisense Compounds

Oligomeric compounds may include, but are not limited to, oligonucleotides, oligonucleosides, oligonucleotide analogs, oligonucleotide mimetics, antisense compounds, antisense oligonucleotides, and siRNAs. An oligomeric compound may be “antisense” to a target nucleic acid, meaning that is capable of undergoing hybridization to a target nucleic acid through hydrogen bonding.

In certain embodiments, an antisense compound is an antisense oligonucleotide. In certain embodiments, the antisense compound is not a ribozyme, a double stranded siRNA, or an shRNA.

In certain embodiments, an antisense compound has a nucleobase sequence that, when written in the 5′ to 3′ direction, comprises the reverse complement of the target segment of a target nucleic acid to which it is targeted. In certain such embodiments, an antisense oligonucleotide has a nucleobase sequence that, when written in the 5′ to 3′ direction, comprises the reverse complement of the target segment of a target nucleic acid to which it is targeted.

In certain embodiments, antisense compounds are 12 to 30 subunits in length. In other words, such antisense compounds are from 12 to 30 linked subunits. In other embodiments, the antisense compound is 8 to 80, 12 to 50, 15 to 30, 18 to 24, 19 to 22, or 20 linked subunits. In certain such embodiments, the antisense compounds are 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, or 80 linked subunits in length, or a range defined by any two of the above values. In some embodiments the antisense compound is an antisense oligonucleotide, and the linked subunits are nucleosides.

In certain embodiments antisense oligonucleotides targeted to a nucleic acid may be shortened or truncated. For example, a single subunit may be deleted from the 5′ end (5′ truncation), or alternatively from the 3′ end (3′ truncation). A shortened or truncated antisense compound targeted to a nucleic acid may have two subunits deleted from the 5′ end, or alternatively may have two subunits deleted from the 3′ end, of the antisense compound. Alternatively, the deleted nucleosides may be dispersed throughout the antisense compound, for example, in an antisense compound having one nucleoside deleted from the 5′ end and one nucleoside deleted from the 3′ end.

When a single additional subunit is present in a lengthened antisense compound, the additional subunit may be located at the 5′ or 3′ end of the antisense compound. When two or more additional subunits are present, the added subunits may be adjacent to each other, for example, in an antisense compound having two subunits added to the 5′ end (5′ addition), or alternatively to the 3′ end (3′ addition), of the antisense compound. Alternatively, the added subunits may be dispersed throughout the antisense compound, for example, in an antisense compound having one subunit added to the 5′ end and one subunit added to the 3′ end.

It is possible to increase or decrease the length of an antisense compound, such as an antisense oligonucleotide, and/or introduce mismatch bases without eliminating activity. For example, in Woolf et al. (Proc. Natl. Acad. Sci. USA 89:7305-7309, 1992), a series of antisense oligonucleotides 13-25 nucleobases in length were tested for their ability to induce cleavage of a target RNA in an oocyte injection model. Antisense oligonucleotides 25 nucleobases in length with 8 or 11 mismatch bases near the ends of the antisense oligonucleotides were able to direct specific cleavage of the target mRNA, albeit to a lesser extent than the antisense oligonucleotides that contained no mismatches. Similarly, target specific cleavage was achieved using 13 nucleobase antisense oligonucleotides, including those with 1 or 3 mismatches.

Gautschi et al (J. Natl. Cancer Inst. 93:463-471, March 2001) demonstrated the ability of an oligonucleotide having 100% complementarity to the bcl-2 mRNA and having 3 mismatches to the bcl-xL mRNA to reduce the expression of both bcl-2 and bcl-xL in vitro and in vivo. Furthermore, this oligonucleotide demonstrated potent anti-tumor activity in vivo.

Maher and Dolnick (Nuc. Acid. Res. 16:3341-3358, 1988) tested a series of tandem 14 nucleobase antisense oligonucleotides, and a 28 and 42 nucleobase antisense oligonucleotides comprised of the sequence of two or three of the tandem antisense oligonucleotides, respectively, for their ability to arrest translation of human DHFR in a rabbit reticulocyte assay. Each of the three 14 nucleobase antisense oligonucleotides alone was able to inhibit translation, albeit at a more modest level than the 28 or 42 nucleobase antisense oligonucleotides.

However, selective reduction of expression of an allelic variant is optimized when the SNP contained in the target nucleic anneals to a complementary base in the antisense compound and not a mismatched base. Moreover, selectivity in general is increased when there are fewer mismatches between the SNP site and the antisense compound. However, a certain number of mismatches may be tolerated.

### Antisense Compound Motifs

In certain embodiments, antisense compounds targeted to a nucleic acid have chemically modified subunits arranged in patterns, or motifs, to confer to the antisense compounds properties such as enhanced the inhibitory activity, increased binding affinity for a target nucleic acid, or resistance to degradation by in vivo nucleases.

Chimeric antisense compounds typically contain at least one region modified so as to confer increased resistance to nuclease degradation, increased cellular uptake, increased binding affinity for the target nucleic acid, and/or increased inhibitory activity. A second region of a chimeric antisense compound may optionally serve as a substrate for the cellular endonuclease RNase H, which cleaves the RNA strand of an RNA:DNA duplex.

Antisense compounds having a gapmer motif are considered chimeric antisense compounds. In a gapmer an internal region having a plurality of nucleotides that supports RNaseH cleavage is positioned between external regions having a plurality of nucleotides that are chemically distinct from the nucleosides of the internal region. In the case of an antisense oligonucleotide having a gapmer motif, the gap segment generally serves as the substrate for endonuclease cleavage, while the wing segments comprise modified nucleosides. In the case of an antisense oligonucleotide for selectively reducing expression of an allelic variant of a gene containing a SNP, the SNP anneals to a nucleobase within the gap segment.

In certain embodiments, the SNP anneals or is complementary to a nucleobase at position 5, 6, 7, 8, 9, 10, 11, 12, 13, or 14 of the antisense oligonucleotide, wherein position refers to the orientation of a nucleobase within the antisense oligonucleotide counting from the 5′ terminus of the antisense oligonucleotide. For example, the 5′ most nucleobase within the antisense oligonucleotide is in the first position of the antisense oligonucleotide. In certain embodiments, the SNP anneals or is complementary to a nucleobase at position 6, 7, 8, 9, or 10 of the antisense oligonucleotide (counting from the 5′ terminus). In certain embodiments, the SNP anneals or is complementary to a nucleobase at position 9 or 10 of the antisense oligonucleotide (counting from the 5′ terminus).

In certain embodiments, the SNP anneals to a nucleobase at position 1, 2, 3, 4, 5, 6, 7, 8, 9, or 10 of the gap segment, wherein position refers to the orientation of a nucleobase within the gap segment counting from the 5′ terminus of the gap segment. For example, the 5′ most nucleobase within the gap segment is in the first position of the gap segment. In certain embodiments, the SNP anneals to a nucleobase at position 4, 5, 6, or 7 counting from the 5′ terminus of the gap segment. In certain embodiments, the SNP anneals to a nucleobase at position 4 or 5 beginning from the 5′ terminus of the gap segment.

In certain embodiments, the regions of a gapmer are differentiated by the types of sugar moieties comprising each distinct region. The types of sugar moieties that are used to differentiate the regions of a gapmer may in some embodiments include β-D-ribonucleosides, β-D-deoxyribonucleosides, 2′-modified nucleosides (such 2′-modified nucleosides may include 2′-MOE, and 2′-O—CH₃, among others), and bicyclic sugar modified nucleosides (such bicyclic sugar modified nucleosides may include those having a 4′-(CH₂)n-O-2′ bridge, where n=1 or n=2). The bicyclic moiety may be a cEt having the formula 4′-CH(CH₃)—O-2.′

The wing-gap-wing motif is frequently described as “X-Y-Z”, where “X” represents the length of the 5′ wing region, “Y” represents the length of the gap region, and “Z” represents the length of the 3′ wing region. As used herein, a gapmer described as “X-Y-Z” has a configuration such that the gap segment is positioned immediately adjacent to each of the 5′ wing segment and the 3′ wing segment. Thus, no intervening nucleotides exist between the 5′ wing segment and gap segment, or the gap segment and the 3′ wing segment. Any of the antisense compounds described herein can have a gapmer motif. In some embodiments, X and Z are the same, in other embodiments they are different. In certain embodiments, Y is between 8 and 15 nucleotides. In certain embodiments, Y is comprised of deoxynucleotides. X, Y or Z can be any of 1, 2, 3, 4, 5, 6, 7, 8, 9, 10, 11, 12, 13, 14, 15, 16, 17, 18, 19, 20, 25, 30 or more nucleotides. Thus, gapmers of the present invention include, but are not limited to, for example 1-10-1, 1-18-1, 2-8-2, 2-9-6, 2-10-2, 2-13-5, 2-16-2, 3-9-3, 3-9-5, 3-10-3, 3-14-3, 4-8-4, 4-9-5, 4-10-5, 4-11-4, 4-12-3, 4-12-4, 5-8-5, 5-9-5, 5-10-4, 5-10-5, or 6-8-6.

In certain embodiments, the antisense compound has a “wingmer” motif, having a wing-gap or gap-wing configuration, i.e. an X-Y or Y-Z configuration as described above for the gapmer configuration. Thus, wingmer configurations of the present invention include, but are not limited to, for example 5-10, 8-4, 4-12, 12-4, 3-14, 16-2, 18-1, 10-3, 2-10, 1-10, 8-2, 2-13, 5-13, 5-8, or 6-8.

In certain embodiments, antisense compounds targeted to a nucleic acid possess a 2-9-6 gapmer motif or a 6-9-2 gapmer motif.

In certain embodiments, antisense compounds targeted to a nucleic acid possess a 3-9-3 gapmer motif.

In certain embodiments, antisense compounds targeted to a nucleic acid possess a 3-9-5 gapmer motif or 5-9-3 gapmer motif.

In certain embodiments, antisense compounds targeted to a nucleic acid possess a 4-9-5 gapmer motif or 5-9-4 gapmer motif.

In certain embodiments, antisense compounds targeted to a nucleic acid possess a 4-10-5 gapmer motif or 5-10-4 gapmer motif.

In certain embodiments, antisense compounds targeted to a nucleic acid possess a 4-11-4 gapmer motif.

In certain embodiments, antisense compounds targeted to a nucleic acid possess a 5-9-5 gapmer motif.

In certain embodiments, antisense compounds targeted to a nucleic acid possess a 5-8-6 gapmer motif or a 6-8-5 gapmer motif.

In certain embodiments, antisense compounds targeted to a nucleic acid possess a 6-7-6 gapmer motif.

In certain embodiments, antisense compounds targeted to a nucleic acid possess a 6-8-5 gapmer motif or a 5-8-6 gapmer motif.

In certain embodiments, antisense compounds targeted to a nucleic acid possess a 3-9-4 gapmer motif or a 4-9-3 gapmer motif.

In certain embodiments, antisense compounds targeted to a nucleic acid possess a 5-7-5 gapmer motif.

In certain embodiments, antisense compounds targeted to a nucleic acid possess a 4-7-4 gapmer motif.

In certain embodiments, antisense compounds targeted to a nucleic acid possess a 5-10-5 gapmer motif.

In certain embodiments, an antisense compound targeted to a nucleic acid has a gap-widened motif.

### Certain Mixed Wings

In certain embodiments, the invention provides gapmer compounds wherein at least one nucleoside of one wing is differently modified compared to at least one other nucleoside of the same wing. Such antisense compounds are referred to as mixed wing antisense compounds (see WO 2008/049085). In certain embodiments, the modifications (or no modification) of one or more nucleosides of the 3′ wing are different from those of one or more other nucleosides of the 3′ wing. Such antisense compounds may be referred to as 3′ mixed wing gapmers. In certain embodiments, the modifications (or no modification) of one or more nucleosides of the 5′ wing are different from those of one or more other nucleosides of the 5′ wing. Such antisense compounds may be referred to as 5′ mixed wing gapmers. In certain embodiments, the modifications (or no modification) of one or more nucleosides of the 3′ wing are different from those of one or more other nucleosides of the 3′ wing and the modifications (or no modification) of one or more nucleosides of the 5′ wing are different from those of one or more other nucleosides of the 5′ wing. Such antisense compounds may be referred to as 3′, 5′ mixed wing gapmers. In such embodiment, the modifications and combination of modifications at the 3′ wing and at the 5′ wing may be the same or they may be different.

In certain embodiments, mixed wing compounds have desirable properties. Certain nucleoside modifications confer on the antisense compound a desirable property, for example increased affinity for a target or nuclease resistance, but also confer an undesirable property, for example increased toxicity. Incorporation of certain other nucleoside modifications results in antisense compounds with different profiles of properties. In certain embodiments, one may combine modifications in one or both wings to optimize desirable characteristics and/or minimize undesirable characteristics. In certain embodiments, the wings of a mixed wing antisense compound comprise one or more nucleoside comprising a first modification that increases affinity of the antisense compound for a target nucleic acid compared to an antisense compound comprising unmodified nucleosides; and one or more nucleoside comprising a second modification that results in reduced toxicity compared to an antisense compound with wings comprising nucleosides that all comprise the first modification.

In certain embodiments, an antisense compound comprises at least one wing comprising at least one MOE substituted nucleoside and at least one high affinity modification. In certain such embodiments, the at least one MOE substituted nucleoside and the at least one high affinity are in the 3′ wing. In certain such embodiments, the at least one MOE substituted nucleoside and the at least one high affinity are in the 5′ wing.

In certain embodiments, an antisense compound comprises 1, 2 or 3 high affinity modifications in the 5′ and/or 3′ wings.

### Target Nucleic Acids, Target Regions and Nucleotide Sequences

In certain embodiments, an allelic variant of huntingtin is selectively reduced. Nucleotide sequences that encode huntingtin include, without limitation, the following: GENBANK Accession No. NT_006081.18, truncated from nucleotides 1566000 to 1768000 (replaced by GENBANK Accession No. NT_006051), incorporated herein as SEQ ID NO: 1, and NM_002111.6, incorporated herein as SEQ ID NO: 2.

It is understood that the sequence set forth in each SEQ ID NO in the Examples contained herein is independent of any modification to a sugar moiety, an internucleoside linkage, or a nucleobase. As such, antisense compounds defined by a SEQ ID NO may comprise, independently, one or more modifications to a sugar moiety, an internucleoside linkage, or a nucleobase. Antisense compounds described by Isis Number (Isis No) indicate a combination of nucleobase sequence and motif.

In certain embodiments, a target region is a structurally defined region of the target nucleic acid. For example, a target region may encompass a 3′ UTR, a 5′ UTR, an exon, an intron, an exon/intron junction, a coding region, a translation initiation region, translation termination region, or other defined nucleic acid region. The structurally defined regions for huntingtin can be obtained by accession number from sequence databases such as NCBI and such information is incorporated herein by reference. In certain embodiments, a target region may encompass the sequence from a 5′ target site of one target segment within the target region to a 3′ target site of another target segment within the same target region.

Targeting includes determination of at least one target segment to which an antisense compound hybridizes, such that a desired effect occurs. In certain embodiments, the desired effect is a reduction in mRNA target nucleic acid levels of a particular allelic variant. In certain embodiments, the desired effect is reduction of levels of the protein encoded by the target nucleic acid or a phenotypic change associated with a particular allelic variant.

A target region may contain one or more target segments. Multiple target segments within a target region may be overlapping. Alternatively, they may be non-overlapping. In certain embodiments, target segments within a target region are separated by no more than about 300 nucleotides. In certain embodiments, target segments within a target region are separated by a number of nucleotides that is, is about, is no more than, is no more than about, 250, 200, 150, 100, 90, 80, 70, 60, 50, 40, 30, 20, or 10 nucleotides on the target nucleic acid, or is a range defined by any two of the preceeding values. In certain embodiments, target segments within a target region are separated by no more than, or no more than about, 5 nucleotides on the target nucleic acid. In certain embodiments, target segments are contiguous. Contemplated are target regions defined by a range having a starting nucleic acid that is any of the 5′ target sites or 3′ target sites listed herein.

Suitable target segments may be found within a 5′ UTR, a coding region, a 3′ UTR, an intron, an exon, or an exon/intron junction. Target segments containing a start codon or a stop codon are also suitable target segments. A suitable target segment may specifically exclude a certain structurally defined region such as the start codon or stop codon.

The determination of suitable target segments may include a comparison of the sequence of a target nucleic acid to other sequences throughout the genome. For example, the BLAST algorithm may be used to identify regions of similarity amongst different nucleic acids. This comparison can prevent the selection of antisense compound sequences that may hybridize in a non-specific manner to sequences other than a selected target nucleic acid (i.e., non-target or off-target sequences).

### Cell Lines

In certain embodiments, the GM04281, GM02171, and GM02173B cell lines are used in experiments described herein below. The GM04281 cell line has a wild-type HTT allele that contains 17 repeats and a mutant HTT allele that contains 69 repeats. The cell line was derived from a patient both of whose parents were also affected by the disease. The GM02171 cell line was chosen as a counter screen control to the GM04281. This cell line was derived from the daughter of parents, only one of whom had the disease. The daughter had not developed HD but was considered to be at risk. The GM02173B cell line was also patient-derived and was used as a haplotype test control.

Table 1 provides SNPs found in the GM04281, GM02171, and GM02173B cell lines. Also provided are the allelic variants found at each SNP position, the genotype for each of the cell lines, and the percentage of HD patients having a particular allelic variant. For example, the two allelic variants for SNP rs6446723 are T and C. The GM02171 cell line is homozygous CC, the GM02173 cell line is heterozygous TC, and the GM04281 cell line is homozygous TT. Fifty percent of HD patients have a T at SNP position rs6446723.

TABLE 1
Allelic Variations for SNPs Associated with HD

| SNP | Variation | GM02171 | GM02173 | GM04281 | TargetPOP | allele |
|---|---|---|---|---|---|---|
| rs6446723 | T/C | CC | TC | TT | 0.50 | T |
| rs3856973 | A/G | AA | AG | GG | 0.50 | G |
| rs2285086 | A/G | GG | AG | AA | 0.50 | A |
| rs363092 | A/C | AA | AC | CC | 0.49 | C |
| rs916171 | C/G | GG | GC | CC | 0.49 | C |
| rs6844859 | T/C | CC | TC | TT | 0.49 | T |
| rs7691627 | A/G | AA | AG | GG | 0.49 | G |
| rs4690073 | A/G | AA | AG | GG | 0.49 | G |
| rs2024115 | A/G | GG | AG | AA | 0.48 | A |
| rs11731237 | T/C | CC | TC | TT | 0.43 | T |
| rs362296 | A/C | AC | AC | AC | 0.42 | C |
| rs10015979 | A/G | AA | AG | GG | 0.42 | G |
| rs7659144 | C/G | CG | CG | CC | 0.41 | C |
| rs363096 | T/C | CC | TC | TT | 0.40 | T |
| rs362273 | A/G | AG | AG | AA | 0.39 | A |
| rs16843804 | T/C | TC | TC | CC | 0.38 | C |
| rs362271 | A/G | AG | AG | GG | 0.38 | G |
| rs362275 | T/C | TC | TC | CC | 0.38 | C |
| rs3121419 | T/C | TC | TC | CC | 0.38 | C |
| rs362272 | A/G | — | AG | GG | 0.38 | G |
| rs3775061 | A/G | AG | AG | AA | 0.38 | A |
| rs34315806 | T/C | TC | TC | CC | 0.38 | C |
| rs363099 | T/C | TC | TC | CC | 0.38 | C |
| rs2298967 | T/C | TC | TC | TT | 0.38 | T |
| rs363088 | A/T | TA | TA | AA | 0.38 | A |
| rs363064 | T/C | TC | TC | CC | 0.35 | C |
| rs363102 | A/G | AA | AA | AA | 0.23 | G |
| rs2798235 | A/G | GG | GG | GG | 0.21 | A |
| rs363080 | T/C | CC | CC | CC | 0.21 | T |
| rs363072 | A/T | TA | AA | AA | 0.13 | A |
| rs363125 | A/C | AC | CC | CC | 0.12 | C |
| rs362303 | T/C | TC | CC | CC | 0.12 | C |
| rs362310 | T/C | TC | CC | CC | 0.12 | C |
| rs10488840 | A/G | AG | GG | GG | 0.12 | G |
| rs362325 | T/C | TC | TT | TT | 0.11 | T |
| rs35892913 | A/G | GG | GG | GG | 0.10 | A |
| rs363102 | A/G | AA | AA | AA | 0.09 | A |
| rs363096 | T/C | CC | TC | TT | 0.09 | C |
| rs11731237 | T/C | CC | TC | TT | 0.09 | C |
| rs10015979 | A/G | AA | AG | GG | 0.08 | A |
| rs363080 | T/C | CC | CC | CC | 0.07 | C |
| rs2798235 | A/G | GG | GG | GG | 0.07 | G |
| rs1936032 | C/G | CC | CC | CC | 0.06 | C |
| rs2276881 | A/G | GG | GG | GG | 0.06 | G |
| rs363070 | A/G | AA | AA | AA | 0.06 | A |
| rs35892913 | A/G | GG | GG | GG | 0.04 | G |
| rs12502045 | T/C | CC | CC | CC | 0.04 | C |
| rs6446723 | T/C | CC | TC | TT | 0.04 | C |
| rs7685686 | A/G | GG | AG | AA | 0.04 | G |
| rs3733217 | T/C | CC | CC | CC | 0.03 | C |
| rs6844859 | T/C | CC | TC | TT | 0.03 | C |
| rs362331 | T/C | CC | TC | TT | 0.03 | C |

### Hybridization

In some embodiments, hybridization occurs between an antisense compound disclosed herein and a SNP site. The most common mechanism of hybridization involves hydrogen bonding (e.g., Watson-Crick, Hoogsteen or reversed Hoogsteen hydrogen bonding) between complementary nucleobases of the nucleic acid molecules.

Hybridization can occur under varying conditions. Stringent conditions are sequence-dependent and are determined by the nature and composition of the nucleic acid molecules to be hybridized.

In certain embodiments, the antisense compounds provided herein are specifically hybridizable with the nucleic acid of a particular allelic variant.

### Complementarity

An antisense compound and a target nucleic acid are complementary to each other when a sufficient number of nucleobases of the antisense compound can hydrogen bond with the corresponding nucleobases of the target nucleic acid, such that a desired effect will occur (e.g., selective reduction of a gene product of an allelic variant).

Non-complementary nucleobases between an antisense compound and a target nucleic acid may be tolerated provided that the antisense compound remains able to specifically hybridize to a target nucleic acid. Moreover, an antisense compound may hybridize over one or more segments of a target nucleic acid such that intervening or adjacent segments are not involved in the hybridization event (e.g., a loop structure, mismatch or hairpin structure).

In certain embodiments, the antisense compounds provided herein, or a specified portion thereof, are, or are at least, 70%, 80%, 85%, 86%, 87%, 88%, 89%, 90%, 91%, 92%, 93%, 94%, 95%, 96%, 97%, 98%, 99%, or 100% complementary to a target nucleic acid, a target region, target segment, SNP site, or specified portion thereof. Percent complementarity of an antisense compound with a target nucleic acid can be determined using routine methods. For example, an antisense compound in which 18 of 20 nucleobases of the antisense compound are complementary to a target region, and would therefore specifically hybridize, would represent 90 percent complementarity. In this example, the remaining noncomplementary nucleobases may be clustered or interspersed with complementary nucleobases and need not be contiguous to each other or to complementary nucleobases. As such, an antisense compound which is 18 nucleobases in length having 4 (four) noncomplementary nucleobases which are flanked by two regions of complete complementarity with the target nucleic acid would have 77.8% overall complementarity with the target nucleic acid and would thus fall within the scope of the present invention. Percent complementarity of an antisense compound with a region of a target nucleic acid can be determined routinely using BLAST programs (basic local alignment search tools) and PowerBLAST programs known in the art (Altschul et al., J. Mol. Biol., 1990, 215, 403 410; Zhang and Madden, Genome Res., 1997, 7, 649 656). Percent homology, sequence identity or complementarity, can be determined by, for example, the Gap program (Wisconsin Sequence Analysis Package, Version 8 for Unix, Genetics Computer Group, University Research Park, Madison Wis.), using default settings, which uses the algorithm of Smith and Waterman (Adv. Appl. Math., 1981, 2, 482 489).

In certain embodiments, the antisense compounds provided herein, or specified portions thereof, are fully complementary (i.e. 100% complementary) to a target nucleic acid, a SNP site, target region, target segment, or specified portion thereof. As used herein, “fully complementary” means each nucleobase of an antisense compound is capable of precise base pairing with the corresponding nucleobases of a target nucleic acid. For example, a 20 nucleobase antisense compound is fully complementary to a target sequence that is 400 nucleobases long, so long as there is a corresponding 20 nucleobase portion of the target nucleic acid that is fully complementary to the antisense compound. Fully complementary can also be used in reference to a specified portion of the first and/or the second nucleic acid. For example, a 20 nucleobase portion of a 30 nucleobase antisense compound can be “fully complementary” to a target sequence that is 400 nucleobases long. The 20 nucleobase portion of the 30 nucleobase oligonucleotide is fully complementary to the target sequence if the target sequence has a corresponding 20 nucleobase portion wherein each nucleobase is complementary to the 20 nucleobase portion of the antisense compound. At the same time, the entire 30 nucleobase antisense compound may or may not be fully complementary to the target sequence, depending on whether the remaining 10 nucleobases of the antisense compound are also complementary to the target sequence.

The location of a non-complementary nucleobase may be at the 5′ end or 3′ end of the antisense compound. Alternatively, the non-complementary nucleobase or nucleobases may be at an internal position of the antisense compound. When two or more non-complementary nucleobases are present, they may be contiguous (i.e. linked) or non-contiguous. In one embodiment, a non-complementary nucleobase is located in the wing segment of a gapmer antisense oligonucleotide.

In certain embodiments, antisense compounds that are, or are up to 12, 13, 14, 15, 16, 17, 18, 19, 20, 21, 22, 23, 24, 25, 26, 27, 28, 29, or 30 nucleobases in length comprise no more than 6, no more than 5, no more than 4, no more than 3, no more than 2, or no more than 1 non-complementary nucleobase(s) relative to a target nucleic acid, SNP site, or specified portion thereof.

In certain embodiments, antisense oligonucleotides that are, or are up to 12, 13, 14, 15, 16, 17, 18, 19, 20, 21, 22, 23, 24, 25, 26, 27, 28, 29, or 30 nucleobases in length comprise no more than 6, no more than 5, no more than 4, no more than 3, no more than 2, or no more than 1 non-complementary nucleobase(s) relative to a target nucleic acid, SNP site, or specified portion thereof.

The antisense compounds provided herein also include those which are complementary to a portion of a target nucleic acid. As used herein, “portion” refers to a defined number of contiguous (i.e. linked) nucleobases within a region or segment of a target nucleic acid. A “portion” can also refer to a defined number of contiguous nucleobases of an antisense compound. In certain embodiments, the antisense compounds, are complementary to at least an 8 nucleobase portion of a target segment. In certain embodiments, the antisense compounds are complementary to at least a 12 nucleobase portion of a target segment. In certain embodiments, the antisense compounds are complementary to at least a 15 nucleobase portion of a target segment. Also contemplated are antisense compounds that are complementary to at least a 9, 10, 11, 12, 13, 14, 15, 16, 17, 18, 19, 20, or more nucleobase portion of a target segment, or a range defined by any two of these values.

### Identity

The antisense compounds provided herein may also have a defined percent identity to a particular nucleotide sequence, SEQ ID NO, or compound represented by a specific Isis number, or portion thereof. As used herein, an antisense compound is identical to the sequence disclosed herein if it has the same nucleobase pairing ability. For example, a RNA which contains uracil in place of thymidine in a disclosed DNA sequence would be considered identical to the DNA sequence since both uracil and thymidine pair with adenine. Shortened and lengthened versions of the antisense compounds described herein as well as compounds having non-identical bases relative to the antisense compounds provided herein also are contemplated. The non-identical bases may be adjacent to each other or dispersed throughout the antisense compound. Percent identity of an antisense compound is calculated according to the number of bases that have identical base pairing relative to the sequence to which it is being compared.

In certain embodiments, the antisense compounds, or portions thereof, are at least 70%, 75%, 80%, 85%, 90%, 95%, 96%, 97%, 98%, 99% or 100% identical to one or more of the antisense compounds or SEQ ID NOs, or a portion thereof, disclosed herein.

In certain embodiments, a portion of the antisense compound is compared to an equal length portion of the target nucleic acid. In certain embodiments, an 8, 9, 10, 11, 12, 13, 14, 15, 16, 17, 18, 19, 20, 21, 22, 23, 24, or 25 nucleobase portion is compared to an equal length portion of the target nucleic acid.

In certain embodiments, a portion of the antisense oligonucleotide is compared to an equal length portion of the target nucleic acid. In certain embodiments, an 8, 9, 10, 11, 12, 13, 14, 15, 16, 17, 18, 19, 20, 21, 22, 23, 24, or 25 nucleobase portion is compared to an equal length portion of the target nucleic acid.

### Modifications

A nucleoside is a base-sugar combination. The nucleobase (also known as base) portion of the nucleoside is normally a heterocyclic base moiety. Nucleotides are nucleosides that further include a phosphate group covalently linked to the sugar portion of the nucleoside. For those nucleosides that include a pentofuranosyl sugar, the phosphate group can be linked to the 2′, 3′ or 5′ hydroxyl moiety of the sugar. Oligonucleotides are formed through the covalent linkage of adjacent nucleosides to one another, to form a linear polymeric oligonucleotide. Within the oligonucleotide structure, the phosphate groups are commonly referred to as forming the internucleoside linkages of the oligonucleotide.

Modifications to antisense compounds encompass substitutions or changes to internucleoside linkages, sugar moieties, or nucleobases. Modified antisense compounds are often preferred over native forms because of desirable properties such as, for example, enhanced cellular uptake, enhanced affinity for nucleic acid target, increased stability in the presence of nucleases, or increased inhibitory activity.

Chemically modified nucleosides may also be employed to increase the binding affinity of a shortened or truncated antisense oligonucleotide for its target nucleic acid. Consequently, comparable results can often be obtained with shorter antisense compounds that have such chemically modified nucleosides.

Chemically modified nucleosides may also be employed to increase selectivity in reducing expression the gene product of an allelic variant.

### Modified Internucleoside Linkages

The naturally occurring internucleoside linkage of RNA and DNA is a 3′ to 5′ phosphodiester linkage. Antisense compounds having one or more modified, i.e. non-naturally occurring, internucleoside linkages are often selected over antisense compounds having naturally occurring internucleoside linkages because of desirable properties such as, for example, enhanced cellular uptake, enhanced affinity for target nucleic acids, and increased stability in the presence of nucleases.

Oligonucleotides having modified internucleoside linkages include internucleoside linkages that retain a phosphorus atom as well as internucleoside linkages that do not have a phosphorus atom. Representative phosphorus containing internucleoside linkages include, but are not limited to, phosphodiesters, phosphotriesters, methylphosphonates, phosphoramidate, and phosphorothioate. Methods of preparation of phosphorous-containing and non-phosphorous-containing linkages are well known.

In certain embodiments, antisense compounds comprise one or more modified internucleoside linkages. In certain embodiments, the modified internucleoside linkages are phosphorothioate linkages. In certain embodiments, each internucleoside linkage of an antisense compound is a phosphorothioate internucleoside linkage.

### Modified Sugar Moieties

Antisense compounds of the invention can optionally contain one or more nucleosides wherein the sugar group has been modified. Such sugar modified nucleosides may impart enhanced nuclease stability, increased binding affinity, increased selectivity for an allelic variant, or some other beneficial biological property to the antisense compounds. In certain embodiments, nucleosides comprise a chemically modified ribofuranose ring moieties. Examples of chemically modified ribofuranose rings include without limitation, addition of substitutent groups (including 5′ and 2′ substituent groups, bridging of non-geminal ring atoms to form bicyclic nucleic acids (BNA), replacement of the ribosyl ring oxygen atom with S, N(R), or C(R1)(R)2(R═H, C1-C12 alkyl or a protecting group) and combinations thereof. Examples of chemically modified sugars include 2′-F-5′-methyl substituted nucleoside (see PCT International Application WO 2008/101157 Published on Aug. 21, 2008 for other disclosed 5′,2′-bis substituted nucleosides) or replacement of the ribosyl ring oxygen atom with S with further substitution at the 2′-position (see published U.S. Patent Application US2005-0130923, published on Jun. 16, 2005) or alternatively 5′-substitution of a BNA (see PCT International Application WO 2007/134181 Published on Nov. 22, 2007 wherein LNA is substituted with for example a 5′-methyl or a 5′-vinyl group).

Examples of nucleosides having modified sugar moieties include without limitation nucleosides comprising 5′-vinyl, 5′-methyl (R or S), 4′-S, 2′-F, 2′-OCH3 and 2′-O(CH2) 2OCH3 substituent groups. The substituent at the 2′ position can also be selected from allyl, amino, azido, thio, O-allyl, O-C1-C10 alkyl, OCF3, O(CH2)2SCH3, O(CH2)2-O—N(Rm)(Rn), and O—CH2-C(═O)—N(Rm)(Rn), where each Rm and Rn is, independently, H or substituted or unsubstituted C1-C10 alkyl.

As used herein, “bicyclic nucleosides” refer to modified nucleosides comprising a bicyclic sugar moiety. Examples of bicyclic nucleosides include without limitation nucleosides comprising a bridge between the 4′ and the 2′ ribosyl ring atoms. In certain embodiments, antisense compounds provided herein include one or more bicyclic nucleosides wherein the bridge comprises a 4′ to 2′ bicyclic nucleoside. Examples of such 4′ to 2′ bicyclic nucleosides, include but are not limited to one of the formulae: 4′-(CH₂)—O-2′ (LNA); 4′-(CH₂)—S-2′; 4′-(CH₂)₂—O-2′ (ENA); 4′-CH(CH₃)—O-2′ and 4′-CH(CH₂OCH₃)—O-2′ (and analogs thereof see U.S. Pat. No. 7,399,845, issued on Jul. 15, 2008); 4′-C(CH₃)(CH₃)—O-2′ (and analogs thereof see published International Application WO/2009/006478, published Jan. 8, 2009); 4′-CH₂—N(OCH₃)-2′ (and analogs thereof see published International Application WO/2008/150729, published Dec. 11, 2008); 4′-CH₂—O—N(CH₃)-2′ (see published U.S. Patent Application US2004-0171570, published Sep. 2, 2004); 4′-CH₂—N(R)—O-2′, wherein R is H, C₁-C₁₂ alkyl, or a protecting group (see U.S. Pat. No. 7,427,672, issued on Sep. 23, 2008); 4′-CH₂—C(H)(CH₃)-2′ (see Chattopadhyaya, et al., J. Org. Chem., 2009, 74, 118-134); and 4′-CH₂—C—(═CH₂)-2′ (and analogs thereof see published International Application WO 2008/154401, published on Dec. 8, 2008). See, for example: Singh et al., Chem. Commun., 1998, 4, 455-456; Koshkin et al., Tetrahedron, 1998, 54, 3607-3630; Wahlestedt et al., Proc. Natl. Acad. Sci. U.S.A, 2000, 97, 5633-5638; Kumar et al., Bioorg. Med. Chem. Lett., 1998, 8, 2219-2222; Singh et al., J. Org. Chem., 1998, 63, 10035-10039; Srivastava et al., J. Am. Chem. Soc., 129 (26) 8362-8379 (Jul. 4, 2007); U.S. Pat. Nos. 7,053,207; 6,268,490; 6,770,748; 6,794,499; 7,034,133; and 6,525,191; Elayadi et al., Curr. Opinion Invens. Drugs, 2001, 2, 558-561; Braasch et al., Chem. Biol., 2001, 8, 1-7; and Orum et al., Curr. Opinion Mol. Ther., 2001, 3, 239-243; and U.S. Pat. No. 6,670,461; International applications WO 2004/106356; WO 94/14226; WO 2005/021570; U.S. Patent Publication Nos. US2004-0171570; US2007-0287831; US2008-0039618; U.S. Pat. No. 7,399,845; U.S. patents Ser. Nos. 12/129,154; 60/989,574; 61/026,995; 61/026,998; 61/056,564; 61/086,231; 61/097,787; 61/099,844; PCT International Applications Nos. PCT/US2008/064591; PCT/US2008/066154; PCT/US2008/068922; and Published PCT International Applications WO 2007/134181. Each of the foregoing bicyclic nucleosides can be prepared having one or more stereochemical sugar configurations including for example α-L-ribofuranose and β-D-ribofuranose (see PCT international application PCT/DK98/00393, published on Mar. 25, 1999 as WO 99/14226).

In certain embodiments, bicyclic sugar moieties of BNA nucleosides include, but are not limited to, compounds having at least one bridge between the 4′ and the 2′ position of the pentofuranosyl sugar moiety wherein such bridges independently comprises 1 or from 2 to 4 linked groups independently selected from —[C(Rₐ)(R_{b})]ₙ—, —C(Rₐ)═C(R_{b})—, —C(Rₐ)═N—, —C(═NRₐ)—, —C(═O)—, —C(═S)—, —O—, —Si(Rₐ)₂—, —S(═O)ₓ—, and —N(Rₐ)—;

wherein:
x is 0, 1, or 2;
n is 1, 2, 3, or 4;
each Rₐ and R_{b} is, independently, H, a protecting group, hydroxyl, C₁-C₁₂ alkyl, substituted C₁-C₁₂ alkyl, C₂-C₁₂ alkenyl, substituted C₂-C₁₂ alkenyl, C₂-C₁₂ alkynyl, substituted C₂-C₁₂ alkynyl, C₅-C₂₀ aryl, substituted C₅-C₂₀ aryl, heterocycle radical, substituted heterocycle radical, heteroaryl, substituted heteroaryl, C₅-C₇ alicyclic radical, substituted C₅-C₇ alicyclic radical, halogen, OJ₁, NJ₁J₂, SJ₁, N₃, COOJ₁, acyl (C(═O)—H), substituted acyl, CN, sulfonyl (S(═O)₂-J₁), or sulfoxyl (S(═O)-J₁); and
each J₁ and J₂ is, independently, H, C₁-C₁₂ alkyl, substituted C₁-C₁₂ alkyl, C₂-C₁₂ alkenyl, substituted C₂-C₁₂ alkenyl, C₂-C₁₂ alkynyl, substituted C₂-C₁₂ alkynyl, C₅-C₂₀ aryl, substituted C₅-C₂₀ aryl, acyl (C(═O)—H), substituted acyl, a heterocycle radical, a substituted heterocycle radical, C₁-C₁₂ aminoalkyl, substituted C₁-C₁₂ aminoalkyl or a protecting group.

In certain embodiments, the bridge of a bicyclic sugar moiety is, —[C(Rₐ)(R_{b})]ₙ—, —[C(Rₐ)(R_{b})]ₙ—O—, —C(RₐR_{b})—N(R)—O— or —C(RₐR_{b})—O—N(R)—. In certain embodiments, the bridge is 4′-CH₂-2′, 4′-(CH₂)₂-2′, 4′-(CH₂)₃-2′, 4′-CH₂—O-2′, 4′-(CH₂)₂—O-2′, 4′-CH₂—O—N(R)-2′ and 4′-CH₂—N(R)—O-2′- wherein each R is, independently, H, a protecting group or C₁-C₁₂ alkyl.

In certain embodiments, bicyclic nucleosides are further defined by isomeric configuration. For example, a nucleoside comprising a 4′-2′ methylene-oxy bridge, may be in the α-L configuration or in the β-D configuration. Previously, α-L-methyleneoxy (4′-CH₂—O-2′) BNA's have been incorporated into antisense oligonucleotides that showed antisense activity (Frieden et al., Nucleic Acids Research, 2003, 21, 6365-6372).

In certain embodiments, bicyclic nucleosides include, but are not limited to, (A) α-L-Methyleneoxy (4′-CH₂—O-2′) BNA, (B) β-D-Methyleneoxy (4′-CH₂—O-2′) BNA, (C) Ethyleneoxy (4′-(CH₂)₂—O-2′) BNA, (D) Aminooxy (4′-CH₂—O—N(R)-2′) BNA, (E) Oxyamino (4′-CH₂—N(R)—O-2′) BNA, and (F) Methyl(methyleneoxy) (4′-CH(CH₃)—O-2′) BNA, (G) methylene-thio (4′-CH₂—S-2′) BNA, (H) methylene-amino (4′-CH2-N(R)-2′) BNA, (I) methyl carbocyclic (4′-CH₂—CH(CH₃)-2′) BNA, (J) propylene carbocyclic (4′-(CH₂)₃-2′) BNA, and (K) ethylene carbocyclic (4′-CH₂—CH₂-2′) (carba LNA or “cLNA”) as depicted below.

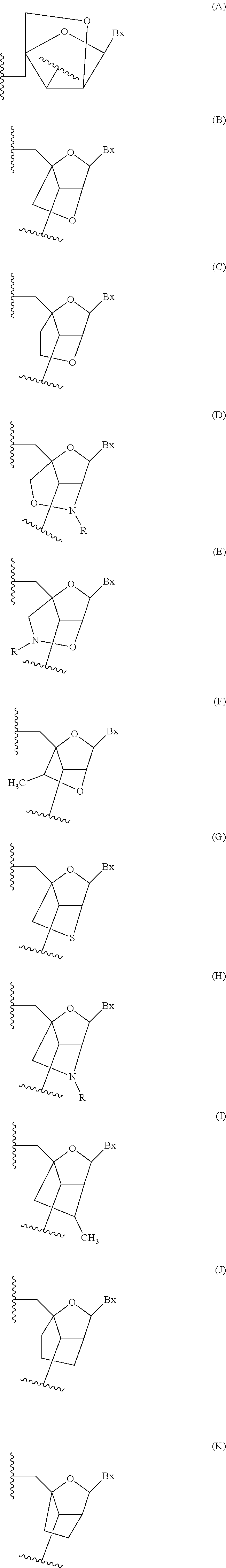

wherein Bx is the base moiety and R is independently H, a protecting group or C₁-C₁₂ alkyl.

In certain embodiments, bicyclic nucleoside having Formula I:

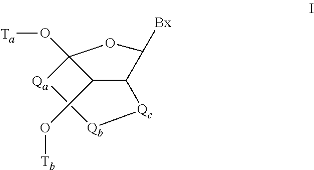

wherein:
Bx is a heterocyclic base moiety;
-Qₐ-Q_{b}-Q_{c}- is —CH₂—N(R_{c})—CH₂—, —C(═O)—N(R_{c})—CH₂—, —CH₂—O—N(R_{c})—, —CH₂—N(R_{c})—O— or —N(R_{c})—O—CH₂;
R_{c} is C₁-C₁₂ alkyl or an amino protecting group; and
Tₐ and T_{b} are each, independently H, a hydroxyl protecting group, a conjugate group, a reactive phosphorus group, a phosphorus moiety or a covalent attachment to a support medium.

In certain embodiments, bicyclic nucleoside having Formula II:

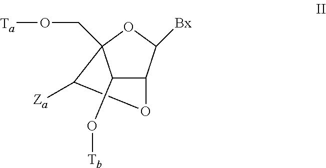

wherein:
Bx is a heterocyclic base moiety;
Tₐ and T_{b} are each, independently H, a hydroxyl protecting group, a conjugate group, a reactive phosphorus group, a phosphorus moiety or a covalent attachment to a support medium;
Zₐ is C₁-C₆ alkyl, C₂-C₆ alkenyl, C₂-C₆ alkynyl, substituted C₁-C₆ alkyl, substituted C₂-C₆ alkenyl, substituted C₂-C₆ alkynyl, acyl, substituted acyl, substituted amide, thiol or substituted thio.

In one embodiment, each of the substituted groups, is, independently, mono or poly substituted with substituent groups independently selected from halogen, oxo, hydroxyl, OJ_{c}, NJ_{c}J_{d}, SJ_{c}, N₃, OC(═X)J_{c}, and NJₑC(═X) NJ_{c}J_{d}, wherein each J_{c}, J_{d} and Jₑ is, independently, H, C₁-C₆ alkyl, or substituted C₁-C₆ alkyl and X is O or NJ_{c}.

In certain embodiments, bicyclic nucleoside having Formula III:

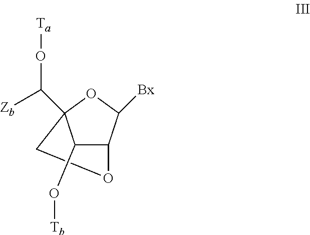

wherein:
Bx is a heterocyclic base moiety;
Tₐ and T_{b} are each, independently H, a hydroxyl protecting group, a conjugate group, a reactive phosphorus group, a phosphorus moiety or a covalent attachment to a support medium;
Z_{b} is C₁-C₆ alkyl, C₂-C₆ alkenyl, C₂-C₆ alkynyl, substituted C₁-C₆ alkyl, substituted C₂-C₆ alkenyl, substituted C₂-C₆ alkynyl or substituted acyl (C(═O)—).

In certain embodiments, bicyclic nucleoside having Formula IV:

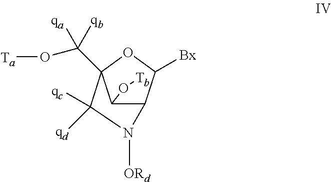

wherein:
Bx is a heterocyclic base moiety;
Tₐ and T_{b} are each, independently H, a hydroxyl protecting group, a conjugate group, a reactive phosphorus group, a phosphorus moiety or a covalent attachment to a support medium;
R_{d} is C₁-C₆ alkyl, substituted C₁-C₆ alkyl, C₂-C₆ alkenyl, substituted C₂-C₆ alkenyl, C₂-C₆ alkynyl or substituted C₂-C₆ alkynyl;
each qₐ, q_{b}, q_{c} and q_{d} is, independently, H, halogen, C₁-C₆ alkyl, substituted C₁-C₆ alkyl, C₂-C₆ alkenyl, substituted C₂-C₆ alkenyl, C₂-C₆ alkynyl or substituted C₂-C₆ alkynyl, C₁-C₆ alkoxyl, substituted C₁-C₆ alkoxyl, acyl, substituted acyl, C₁-C₆ aminoalkyl or substituted C₁-C₆ aminoalkyl;

In certain embodiments, bicyclic nucleoside having Formula V:

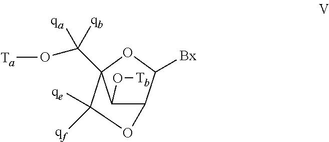

wherein:
Bx is a heterocyclic base moiety;
Tₐ and T_{b} are each, independently H, a hydroxyl protecting group, a conjugate group, a reactive phosphorus group, a phosphorus moiety or a covalent attachment to a support medium;
qₐ, q_{b}, qₑ and q_{f} are each, independently, hydrogen, halogen, C₁-C₁₂ alkyl, substituted C₁-C₁₂ alkyl, C₂-C₁₂ alkenyl, substituted C₂-C₁₂ alkenyl, C₂-C₁₂ alkynyl, substituted C₂-C₁₂ alkynyl, C₁-C₁₂ alkoxy, substituted C₁-C₁₂ alkoxy, OJⱼ, SJⱼ, SOJⱼ, SO₂Jⱼ, NJⱼJₖ, N₃, CN, C(═O)OJⱼ, C(═O)NJⱼJₖ, C(═O)Jⱼ, O—C(═O)NJⱼJₖ, N(H)C(═NH)NJⱼJₖ, N(H)C(═O)NJⱼJₖ or N(H)C(═S)NJⱼJₖ;
or qₑ and q_{f} together are ═C(q_{g})(qₕ);
q_{g} and qₕ are each, independently, H, halogen, C₁-C₁₂ alkyl or substituted C₁-C₁₂ alkyl.

The synthesis and preparation of the methyleneoxy (4′-CH₂—O-2′) BNA monomers adenine, cytosine, guanine, 5-methyl-cytosine, thymine and uracil, along with their oligomerization, and nucleic acid recognition properties have been described (Koshkin et al., Tetrahedron, 1998, 54, 3607-3630). BNAs and preparation thereof are also described in WO 98/39352 and WO 99/14226.

Analogs of methyleneoxy (4′-CH₂—O-2′) BNA, methyleneoxy (4′-CH₂—O-2′) BNA and 2′-thio-BNAs, have also been prepared (Kumar et al., Bioorg. Med. Chem. Lett., 1998, 8, 2219-2222). Preparation of locked nucleoside analogs comprising oligodeoxyribonucleotide duplexes as substrates for nucleic acid polymerases has also been described (Wengel et al., WO 99/14226). Furthermore, synthesis of 2′-amino-BNA, a novel conformationally restricted high-affinity oligonucleotide analog has been described in the art (Singh et al., J. Org. Chem., 1998, 63, 10035-10039). In addition, 2′-Amino- and 2′-methylamino-BNA's have been prepared and the thermal stability of their duplexes with complementary RNA and DNA strands has been previously reported.

In certain embodiments, bicyclic nucleoside having Formula VI:

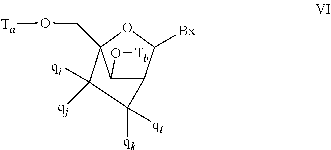

wherein:
Bx is a heterocyclic base moiety;
Tₐ and T_{b} are each, independently H, a hydroxyl protecting group, a conjugate group, a reactive phosphorus group, a phosphorus moiety or a covalent attachment to a support medium;
each qᵢ, qⱼ, qₖ and qₗ is, independently, H, halogen, C₁-C₁₂ alkyl, substituted C₁-C₁₂ alkyl, C₂-C₁₂ alkenyl, substituted C₂-C₁₂ alkenyl, C₂-C₁₂ alkynyl, substituted C₂-C₁₂ alkynyl, C₁-C₁₂ alkoxyl, substituted C₁-C₁₂ alkoxyl, OJⱼ, SJⱼ, SOJⱼ, SO₂Jⱼ, NJⱼJₖ, N₃, CN, C(—O)OJⱼ, C(═O)NJⱼJₖ, C(═O)Jⱼ, O—C(═O)NJⱼJₖ, N(H)C(═NH)NJⱼJₖ, N(H)C(═O)NJⱼJₖ or N(H)C(═S)NJⱼJₖ; and
qᵢ and qⱼ or qₗ and qₖ together are ═C(q_{g})(qₕ), wherein q_{g} and qₕ are each, independently, H, halogen, C₁-C₁₂ alkyl or substituted C₁-C₁₂ alkyl.

One carbocyclic bicyclic nucleoside having a 4′-(CH₂)₃-2′ bridge and the alkenyl analog bridge 4′-CH═CH—CH₂-2′ have been described (Frier et al., Nucleic Acids Research, 1997, 25 (22), 4429-4443 and Albaek et al., J. Org. Chem., 2006, 71, 7731-7740). The synthesis and preparation of carbocyclic bicyclic nucleosides along with their oligomerization and biochemical studies have also been described (Srivastava et al., J. Am. Chem. Soc. 2007, 129 (26), 8362-8379).

As used herein, “4′-2′ bicyclic nucleoside” or “4′ to 2′ bicyclic nucleoside” refers to a bicyclic nucleoside comprising a furanose ring comprising a bridge connecting two carbon atoms of the furanose ring connects the 2′ carbon atom and the 4′ carbon atom of the sugar ring.

As used herein, “monocylic nucleosides” refer to nucleosides comprising modified sugar moieties that are not bicyclic sugar moieties. In certain embodiments, the sugar moiety, or sugar moiety analogue, of a nucleoside may be modified or substituted at any position.

As used herein, “2′-modified sugar” means a furanosyl sugar modified at the 2′ position. In certain embodiments, such modifications include substituents selected from: a halide, including, but not limited to substituted and unsubstituted alkoxy, substituted and unsubstituted thioalkyl, substituted and unsubstituted amino alkyl, substituted and unsubstituted alkyl, substituted and unsubstituted allyl, and substituted and unsubstituted alkynyl. In certain embodiments, 2′ modifications are selected from substituents including, but not limited to: O[(CH₂)ₙO]ₘCH₃, O(CH₂)ₙNH₂, O(CH₂)ₙCH₃, O(CH₂)ₙONH₂, OCH₂C(═O)N(H)CH₃, and O(CH₂)ₙON [(CH₂)ₙCH₃]₂, where n and m are from 1 to about 10. Other 2′-substituent groups can also be selected from: C₁-C₁₂ alkyl, substituted alkyl, alkenyl, alkynyl, alkaryl, aralkyl, O-alkaryl or O-aralkyl, SH, SCH₃, OCN, Cl, Br, CN, CF₃, OCF₃, SOCH₃, SO₂CH₃, ONO₂, NO₂, N₃, NH₂, heterocycloalkyl, heterocycloalkaryl, aminoalkylamino, polyalkylamino, substituted silyl, an RNA cleaving group, a reporter group, an intercalator, a group for improving pharmacokinetic properties, or a group for improving the pharmacodynamic properties of an antisense compound, and other substituents having similar properties. In certain embodiments, modified nucleosides comprise a 2′-MOE side chain (Baker et al., J. Biol. Chem., 1997, 272, 11944-12000). Such 2′-MOE substitution have been described as having improved binding affinity compared to unmodified nucleosides and to other modified nucleosides, such as 2′-O-methyl, O-propyl, and O-aminopropyl. Oligonucleotides having the 2′-MOE substituent also have been shown to be antisense inhibitors of gene expression with promising features for in vivo use (Martin, P., Helv. Chim. Acta, 1995, 78, 486-504; Altmann et al., Chimia, 1996, 50, 168-176; Altmann et al., Biochem. Soc. Trans., 1996, 24, 630-637; and Altmann et al., Nucleosides Nucleotides, 1997, 16, 917-926).

As used herein, a “modified tetrahydropyran nucleoside” or “modified THP nucleoside” means a nucleoside having a six-membered tetrahydropyran “sugar” substituted in for the pentofuranosyl residue in normal nucleosides (a sugar surrogate). Modified THP nucleosides include, but are not limited to, what is referred to in the art as hexitol nucleic acid (HNA), anitol nucleic acid (ANA), manitol nucleic acid (MNA) (see Leumann, CJ. Bioorg. & Med. Chem. (2002) 10:841-854), fluoro HNA (F-HNA) or those compounds having Formula X:

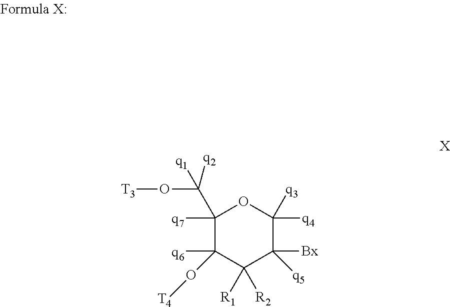

wherein independently for each of said at least one tetrahydropyran nucleoside analog of Formula X:
Bx is a heterocyclic base moiety;
T₃ and T₄ are each, independently, an internucleoside linking group linking the tetrahydropyran nucleoside analog to the antisense compound or one of T₃ and T₄ is an internucleoside linking group linking the tetrahydropyran nucleoside analog to the antisense compound and the other of T₃ and T₄ is H, a hydroxyl protecting group, a linked conjugate group or a 5′ or 3′-terminal group;
q₁, q₂, q₃, q₄, q₅, q₆ and q₇ are each independently, H, C₁-C₆ alkyl, substituted C₁-C₆ alkyl, C₂-C₆ alkenyl, substituted C₂-C₆ alkenyl, C₂-C₆ alkynyl or substituted C₂-C₆ alkynyl; and
one of R₁ and R₂ is hydrogen and the other is selected from halogen, substituted or unsubstituted alkoxy, NJ₁J₂, SJ₁, N₃, OC(═X)J₁, OC(═X)NJ₁J₂, NJ₃C(═X)NJ₁J₂ and CN, wherein X is O, S or NJ₁ and each J₁, J₂ and J₃ is, independently, H or C₁-C₆ alkyl.

In certain embodiments, the modified THP nucleosides of Formula X are provided wherein qₘ, qₙ, qₚ, qᵣ, qₛ, qₜ and qᵤ are each H. In certain embodiments, at least one of qₘ, qₙ, qₚ, qᵣ, qₛ, qₜ and qᵤ is other than H. In certain embodiments, at least one of qₘ, qₙ, qₚ, qᵣ, qₛ, qₜ and qᵤ is methyl. In certain embodiments, THP nucleosides of Formula X are provided wherein one of R₁ and R₂ is F. In certain embodiments, R₁ is fluoro and R₂ is H; R₁ is methoxy and R₂ is H, and R₁ is methoxyethoxy and R₂ is H.

As used herein, “2′-modified” or “2′-substituted” refers to a nucleoside comprising a sugar comprising a substituent at the 2′ position other than H or OH. 2′-modified nucleosides, include, but are not limited to, bicyclic nucleosides wherein the bridge connecting two carbon atoms of the sugar ring connects the 2′ carbon and another carbon of the sugar ring; and nucleosides with non-bridging 2′substituents, such as allyl, amino, azido, thio, O-allyl, O—C₁-C₁₀ alkyl, —OCF₃, O—(CH₂)₂—O-CH₃, 2′-O(CH₂)₂SCH₃, O—(CH₂)₂—O—N(Rₘ)(Rₙ), or O—CH₂—C(═O)−N(Rₘ)(Rₙ), where each Rₘ and Rₙ is, independently, H or substituted or unsubstituted C₁-C₁₀ alkyl. 2′-modified nucleosides may further comprise other modifications, for example at other positions of the sugar and/or at the nucleobase.

As used herein, “2′-F” refers to a nucleoside comprising a sugar comprising a fluoro group at the 2′ position.

As used herein, “2′-OMe” or “2′-OCH₃” or “2′-O-methyl” each refers to a nucleoside comprising a sugar comprising an —OCH₃ group at the 2′ position of the sugar ring.

As used herein, “MOE” or “2′-MOE” or “2′-OCH₂CH₂OCH₃” or “2′-O-methoxyethyl” each refers to a nucleoside comprising a sugar comprising a —OCH₂CH₂OCH₃ group at the 2′ position of the sugar ring.

As used herein, “oligonucleotide” refers to a compound comprising a plurality of linked nucleosides. In certain embodiments, one or more of the plurality of nucleosides is modified. In certain embodiments, an oligonucleotide comprises one or more ribonucleosides (RNA) and/or deoxyribonucleosides (DNA).

Many other bicyclo and tricyclo sugar surrogate ring systems are also know in the art that can be used to modify nucleosides for incorporation into antisense compounds (see for example review article: Leumann, J. C, Bioorganic & Medicinal Chemistry, 2002, 10, 841-854). Such ring systems can undergo various additional substitutions to enhance activity.

Methods for the preparations of modified sugars are well known to those skilled in the art.

In nucleotides having modified sugar moieties, the nucleobase moieties (natural, modified or a combination thereof) are maintained for hybridization with an appropriate nucleic acid target.

In certain embodiments, antisense compounds comprise one or more nucleotides having modified sugar moieties. In certain embodiments, the modified sugar moiety is 2′-MOE. In certain embodiments, the 2′-MOE modified nucleotides are arranged in a gapmer motif. In certain embodiments, the modified sugar moiety is a cEt. In certain embodiments, the cEt modified nucleotides are arranged throughout the wings of a gapmer motif.

### Modified Nucleobases

Nucleobase (or base) modifications or substitutions are structurally distinguishable from, yet functionally interchangeable with, naturally occurring or synthetic unmodified nucleobases. Both natural and modified nucleobases are capable of participating in hydrogen bonding. Such nucleobase modifications may impart nuclease stability, binding affinity, increased selectivity for an allelic variant, or some other beneficial biological property to antisense compounds. Modified nucleobases include synthetic and natural nucleobases such as, for example, 5-methylcytosine (5-me-C). Certain nucleobase substitutions, including 5-methylcytosine substitutions, are particularly useful for increasing the binding affinity of an antisense compound for a target nucleic acid. For example, 5-methylcytosine substitutions have been shown to increase nucleic acid duplex stability by 0.6-1.2° C. (Sanghvi, Y. S., Crooke, S. T. and Lebleu, B., eds., Antisense Research and Applications, CRC Press, Boca Raton, 1993, pp. 276-278).

Additional modified nucleobases include 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl (—C≡C—CH₃) uracil and cytosine and other alkynyl derivatives of pyrimidine bases, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 2-F-adenine, 2-aminoadenine, 8-azaguanine and 8-azaadenine, 7-deazaguanine and 7-deazaadenine and 3-deazaguanine and 3-deazaadenine.

Heterocyclic base moieties may also include those in which the purine or pyrimidine base is replaced with other heterocycles, for example 7-deaza-adenine, 7-deazaguanosine, 2-aminopyridine and 2-pyridone. Nucleobases that are particularly useful for increasing the binding affinity of antisense compounds include 5-substituted pyrimidines, 6-azapyrimidines and N-2, N-6 and O-6 substituted purines, including 2 aminopropyladenine, 5-propynyluracil and 5-propynylcytosine.

In certain embodiments, antisense compounds comprise one or more modified nucleobases. In certain embodiments, gap-widened antisense oligonucleotides comprise one or more modified nucleobases. In certain embodiments, the modified nucleobase is 5-methylcytosine. In certain embodiments, each cytosine is a 5-methylcytosine.

### Compositions and Methods for Formulating Pharmaceutical Compositions

Antisense oligonucleotides may be admixed with pharmaceutically acceptable active or inert substances for the preparation of pharmaceutical compositions or formulations.

Compositions and methods for the formulation of pharmaceutical compositions are dependent upon a number of criteria, including, but not limited to, route of administration, extent of disease, or dose to be administered.

An antisense compound can be utilized in pharmaceutical compositions by combining the antisense compound with a suitable pharmaceutically acceptable diluent or carrier. A pharmaceutically acceptable diluent includes phosphate-buffered saline (PBS). PBS is a diluent suitable for use in compositions to be delivered parenterally. Accordingly, in one embodiment, employed in the methods described herein is a pharmaceutical composition comprising an antisense compound and a pharmaceutically acceptable diluent. In certain embodiments, the pharmaceutically acceptable diluent is PBS. In certain embodiments, the antisense compound is an antisense oligonucleotide.

Pharmaceutical compositions comprising antisense compounds encompass any pharmaceutically acceptable salts, esters, or salts of such esters, or any other oligonucleotide which, upon administration to an animal, including a human, is capable of providing (directly or indirectly) the biologically active metabolite or residue thereof. Accordingly, for example, the disclosure is also drawn to pharmaceutically acceptable salts of antisense compounds, prodrugs, pharmaceutically acceptable salts of such prodrugs, and other bioequivalents. Suitable pharmaceutically acceptable salts include, but are not limited to, sodium and potassium salts.

A prodrug can include the incorporation of additional nucleosides at one or both ends of an antisense compound which are cleaved by endogenous nucleases within the body, to form the active antisense compound.

### Conjugated Antisense Compounds

Antisense compounds may be covalently linked to one or more moieties or conjugates which enhance the activity, cellular distribution, increased selectivity for an allelic variant, or cellular uptake of the resulting antisense oligonucleotides. Typical conjugate groups include cholesterol moieties and lipid moieties. Additional conjugate groups include carbohydrates, phospholipids, biotin, phenazine, folate, phenanthridine, anthraquinone, acridine, fluoresceins, rhodamines, coumarins, and dyes.

Antisense compounds can also be modified to have one or more stabilizing groups that are generally attached to one or both termini of antisense compounds to enhance properties such as, for example, nuclease stability. Included in stabilizing groups are cap structures. These terminal modifications protect the antisense compound having terminal nucleic acid from exonuclease degradation, and can help in delivery and/or localization within a cell. The cap can be present at the 5′-terminus (5′-cap), or at the 3′-terminus (3′-cap), or can be present on both termini. Cap structures are well known in the art and include, for example, inverted deoxy abasic caps. Further 3′ and 5′-stabilizing groups that can be used to cap one or both ends of an antisense compound to impart nuclease stability include those disclosed in WO 03/004602 published on Jan. 16, 2003.

### Cell Culture and Antisense Compounds Treatment

The effects of antisense compounds on the level, activity or expression target nucleic acids can be tested in vitro in a variety of cell types. Cell types used for such analyses are available from commercial vendors (e.g. American Type Culture Collection, Manassas, VA; Zen-Bio, Inc., Research Triangle Park, NC; Clonetics Corporation, Walkersville, MD) and are cultured according to the vendor's instructions using commercially available reagents (e.g. Invitrogen Life Technologies, Carlsbad, CA). Illustrative cell types include, but are not limited to, HepG2 cells, Hep3B cells, and primary hepatocytes. Illustrative cell lines include GM04281, GM02171, and GM02173B cells.

### In Vitro Testing of Antisense Oligonucleotides

Described herein are methods for treatment of cells with antisense oligonucleotides, which can be modified appropriately for treatment with other antisense compounds.

In general, cells are treated with antisense oligonucleotides when the cells reach approximately 60-80% confluency in culture.

One reagent commonly used to introduce antisense oligonucleotides into cultured cells includes the cationic lipid transfection reagent LIPOFECTIN (Invitrogen, Carlsbad, CA). Antisense oligonucleotides are mixed with LIPOFECTIN in OPTI-MEM 1 (Invitrogen, Carlsbad, CA) to achieve the desired final concentration of antisense oligonucleotide and a LIPOFECTIN concentration that typically ranges 2 to 12 μg/mL per 100 nM antisense oligonucleotide.

Another reagent used to introduce antisense oligonucleotides into cultured cells includes LIPOFECTAMINE (Invitrogen, Carlsbad, CA). Antisense oligonucleotide is mixed with LIPOFECTAMINE in OPTI-MEM 1 reduced serum medium (Invitrogen, Carlsbad, CA) to achieve the desired concentration of antisense oligonucleotide and a LIPOFECTAMINE concentration that typically ranges 2 to 12 μg/mL per 100 nM antisense oligonucleotide.

Another technique used to introduce antisense oligonucleotides into cultured cells includes electroporation.

Cells are treated with antisense oligonucleotides by routine methods. Cells are typically harvested 16-24 hours after antisense oligonucleotide treatment, at which time RNA or protein levels of target nucleic acids are measured by methods known in the art and described herein. In general, when treatments are performed in multiple replicates, the data are presented as the average of the replicate treatments.

The concentration of antisense oligonucleotide used varies from cell line to cell line. Methods to determine the optimal antisense oligonucleotide concentration for a particular cell line are well known in the art. Antisense oligonucleotides are typically used at concentrations ranging from 1 nM to 300 nM when transfected with LIPOFECTAMINE. Antisense oligonucleotides are used at higher concentrations ranging from 625 to 20,000 nM when transfected using electroporation.

### RNA Isolation

RNA analysis can be performed on total cellular RNA or poly (A)+ mRNA. Methods of RNA isolation are well known in the art. RNA is prepared using methods well known in the art, for example, using the TRIZOL Reagent (Invitrogen, Carlsbad, CA) according to the manufacturer's recommended protocols.

### Analysis of Inhibition of Target Levels or Expression

Reduction, inhibition, or expression of a target nucleic acid can be assayed in a variety of ways known in the art. For example, target nucleic acid levels can be quantitated by, e.g., Northern blot analysis, competitive polymerase chain reaction (PCR), or quantitative real-time PCR. RNA analysis can be performed on total cellular RNA or poly (A)+ mRNA. Methods of RNA isolation are well known in the art. Northern blot analysis is also routine in the art. Quantitative real-time PCR can be conveniently accomplished using the commercially available ABI PRISM 7600, 7700, or 7900 Sequence Detection System, available from PE-Applied Biosystems, Foster City, CA and used according to manufacturer's instructions.

### Quantitative Real-Time PCR Analysis of Target RNA Levels

Quantitation of target RNA levels may be accomplished by quantitative real-time PCR using the ABI PRISM 7600, 7700, or 7900 Sequence Detection System (PE-Applied Biosystems, Foster City, CA) according to manufacturer's instructions. Methods of quantitative real-time PCR are well known in the art.

Prior to real-time PCR, the isolated RNA is subjected to a reverse transcriptase (RT) reaction, which produces complementary DNA (cDNA) that is then used as the substrate for the real-time PCR amplification. The RT and real-time PCR reactions are performed sequentially in the same sample well. RT and real-time PCR reagents are obtained from Invitrogen (Carlsbad, CA). RT real-time-PCR reactions are carried out by methods well known to those skilled in the art.

Gene (or RNA) target quantities obtained by real time PCR are normalized using either the expression level of a gene whose expression is constant, such as cyclophilin A, or by quantifying total RNA using RIBOGREEN (Invitrogen, Inc. Carlsbad, CA). Cyclophilin A expression is quantified by real time PCR, by being run simultaneously with the target, multiplexing, or separately. Total RNA is quantified using RIBOGREEN RNA quantification reagent (Invetrogen, Inc. Eugene, OR). Methods of RNA quantification by RIBOGREEN are taught in Jones, L. J., et al, (Analytical Biochemistry, 1998, 265, 368-374). A CYTOFLUOR 4000 instrument (PE Applied Biosystems) is used to measure RIBOGREEN fluorescence.

Probes and primers are designed to hybridize to target nucleic acids. Methods for designing real-time PCR probes and primers are well known in the art, and may include the use of software such as PRIMER EXPRESS Software (Applied Biosystems, Foster City, CA).

### Analysis of Protein Levels

Reduction, inhibition, or expression of target nucleic acids can be assessed by measuring target protein levels. Target protein levels can be evaluated or quantitated in a variety of ways well known in the art, such as immunoprecipitation, Western blot analysis (immunoblotting), enzyme-linked immunosorbent assay (ELISA), quantitative protein assays, protein activity assays (for example, caspase activity assays), immunohistochemistry, immunocytochemistry or fluorescence-activated cell sorting (FACS). Antibodies directed to a target can be identified and obtained from a variety of sources, such as the MSRS catalog of antibodies (Aerie Corporation, Birmingham, MI), or can be prepared via conventional monoclonal or polyclonal antibody generation methods well known in the art. Antibodies useful for the detection of mouse, rat, monkey, and human proteins are commercially available.

### In Vivo Testing of Antisense Compounds

Antisense compounds, for example, antisense oligonucleotides, are tested in animals to assess their ability to selectively reduce or inhibit expression of target gene product and produce phenotypic changes, such as, amelioration of a disease symptom. Testing may be performed in normal animals, or in experimental disease models. For administration to animals, antisense oligonucleotides are formulated in a pharmaceutically acceptable diluent, such as phosphate-buffered saline. Administration includes parenteral routes of administration, such as intraperitoneal, intravenous, and subcutaneous. Calculation of antisense oligonucleotide dosage and dosing frequency is within the abilities of those skilled in the art, and depends upon factors such as route of administration and animal body weight. Following a period of treatment with antisense oligonucleotides, RNA or protein is isolated from tissue and changes in target nucleic acid or protein expression are measured.

### Administration

In certain embodiments, the compounds and compositions described herein may be administered in a number of ways depending upon whether local or systemic treatment is desired and upon the area to be treated. Administration may be topical (including ophthalmic, vaginal, rectal, intranasal), oral, pulmonary (including by inhalation or insufflation of powders or aerosols, including by nebulizer; intratracheal, intranasal, epidermal and transdermal) or parenteral, for example, by intravenous drip, intravenous injection or subcutaneous, intraperitoneal, intraocular, intravitreal, or intramuscular injection.

In certain embodiments, the compounds and compositions as described herein are administered parenterally.

In certain embodiments, parenteral administration is by infusion. Infusion can be chronic or continuous or short or intermittent. In certain embodiments, infused pharmaceutical agents are delivered with a pump. In certain embodiments, parenteral administration is by injection.

In certain embodiments, compounds and compositions are delivered to the CNS. In certain embodiments, compounds and compositions are delivered to the cerebrospinal fluid. In certain embodiments, compounds and compositions are administered to the brain parenchyma. In certain embodiments, compounds and compositions are delivered to an animal by intrathecal administration, or intracerebroventricular administration. Broad distribution of compounds and compositions, described herein, within the central nervous system may be achieved with intraparenchymal administration, intrathecal administration, or intracerebroventricular administration.

In certain embodiments, parenteral administration is by injection. The injection may be delivered with a syringe or a pump. In certain embodiments, the injection is a bolus injection. In certain embodiments, the injection is administered directly to a tissue, such as striatum, caudate, cortex, hippocampus and cerebellum.

In certain embodiments, methods of specifically localizing a pharmaceutical agent, such as by bolus injection, decreases median effective concentration (EC50) by a factor of 20, 25, 30, 35, 40, 45 or 50. In certain embodiments, the pharmaceutical agent in an antisense compound as further described herein. In certain embodiments, the targeted tissue is brain tissue. In certain embodiments the targeted tissue is striatal tissue. In certain embodiments, decreasing EC50 is desirable because it reduces the dose required to achieve a pharmacological result in a patient in need thereof.

In certain embodiments, an antisense oligonucleotide is delivered by injection or infusion once every month, every two months, every 90 days, every 3 months, every 6 months, twice a year or once a year.

### Certain Compounds and Indications

Provided herein are compounds and methods that provide potent inhibition and increased selectivity for a mutant allele. Potency is demonstrated by the percent inhibition of mutant mRNA achieved by the antisense oligonucleotides targeting a SNP compared to the percent inhibition of mutant mRNA achieved by the benchmark oligonucleotide. Selectivity is demonstrated by the ability of the antisense oligonucleotide targeting a SNP to inhibit expression of the major allele or mutant allele preferentially compared to the minor allele or wild type allele. The usage of three cell lines with different genotypes at each SNP position have facilitated the determination of design rules that provide for potent and selective SNP targeting antisense oligonucleotides.

In certain embodiments, the compounds are antisense oligonucleotides as further described herein. The antisense oligonucleotides preferentially target a SNP or differentiating polymorphism. Oligonucleotides of various lengths were tested and certain lengths were determined to be beneficial for the targeting of SNPs.

In certain embodiments, the antisense oligonucleotides have a sequence that is 12-30 nucleobases in length. In certain embodiments, the antisense oligonucleotides have a sequence that is 12-25 nucleobases in length. In certain embodiments, the antisense oligonucleotides have a sequence that is 12-21 nucleobases in length. In certain embodiments, the antisense oligonucleotides have a sequence that is 12-20 nucleobases in length. In certain embodiments, the antisense oligonucleotides have a sequence that is 13-20 nucleobases in length. In certain embodiments, the antisense oligonucleotides have a sequence that is 14-20 nucleobases in length. In certain embodiments, the antisense oligonucleotides have a sequence that is 15-20 nucleobases in length. In certain embodiments, the antisense oligonucleotides have a sequence that is 12-19 nucleobases in length. In certain embodiments, the antisense oligonucleotides have a sequence that is 13-19 nucleobases in length. In certain embodiments, the antisense oligonucleotides have a sequence that is 14-19 nucleobases in length. In certain embodiments, the antisense oligonucleotides have a sequence that is 15-19, nucleobases in length. In certain embodiments, the antisense oligonucleotides have a sequence that is 16-19 nucleobases in length. In certain embodiments, the antisense oligonucleotides have a sequence that is 17-19 nucleobases in length. In certain embodiments, the antisense oligonucleotides have a sequence that is 12, 13, 14, 15, 16, 17, 18, 19, 21, 22, 23, 24, 25, 26, 27, 28, 29, 30 nucleobases in length.

For oligonucleotides of various lengths, the position of the nucleoside complementary to the SNP position was shifted within the gap and the wings and the effect was tested. Certain positions within the antisense oligonucleotide are shown to be beneficial for targeting SNPs.

In certain embodiments, the antisense oligonucleotide is at least 12, at least 13, at least 14, at least 15, at least 16, at least 17 at least 18 or at least 19 nucleobases in length and the SNP is complementary to positions 6-15 counting from the 5′ terminus of the antisense oligonucleotide and/or positions 1-9 counting from the 5′ end of the gap. In certain embodiments, the antisense oligonucleotide is at least 12, at least 13, at least 14, at least 15, at least 16, at least 17 at least 18 or at least 19 nucleobases in length and the SNP is complementary to positions 8-14 counting from the 5′ terminus of the antisense oligonucleotide and/or positions 1-9 counting from the 5′ end of the gap. In certain embodiments, the antisense oligonucleotide is at least 12, at least 13, at least 14, at least 15, at least 16, at least 17 at least 18 or at least 19 nucleobases in length and the SNP is complementary to positions 8-14 counting from the 5′ terminus of the antisense oligonucleotide and/or positions 4-7 counting from the 5′ end of the gap. In certain embodiments, the antisense oligonucleotide is at least 12, at least 13, at least 14, at least 15, at least 16, at least 17 at least 18 or at least 19 nucleobases in length and the SNP is complementary to positions 8-10 counting from the 5′ terminus of the antisense oligonucleotide and/or positions 4-6 counting from the 5′ end of the gap.

In certain embodiments, the SNP is complementary to position 8, 9, or 10 counting from the 5′ terminus of the oligonucleotide or position 4, 5, or 6, counting from the 5′ end of the gap. For oligonucleotides of various lengths, the effect of the length of the gap, 5′ wing, and 3′ wing was tested.

Certain wing-gap-wing combinations were shown to be beneficial for a SNP targeting antisense oligonucleotide. In certain embodiments the gap is 7-11 nucleobases in length and each wing is independently 1-6 nucleobases in length. In certain embodiments the gap is 7-11 nucleobases in length and each wing is independently 2-6 nucleobases in length. In certain embodiments the gap is 8-11 nucleobases in length and each wing is independently 2-6 nucleobases in length. In certain embodiments the gap is 9-11 nucleobases in length and each wing is independently 2-6 nucleobases in length. In certain embodiments the gap is 9 nucleobases in length and each wing is independently 2-6 nucleobases in length. In certain embodiments the gap is 10 nucleobases in length and each wing is independently 2-6 or 4-5 nucleobases in length. In certain embodiments the gap is 11 nucleobases in length and each wing is independently 2-6, or 4-5 nucleobases in length. In certain embodiments, the wing-gap-wing configuration is one of 4-7-4, 5-8-6, 6-8-5, 6-7-6, 5-7-5. 6-8-5, 5-8-6, 3-9-4, 4-9-3, 2-9-6, 6,9,2,3-9-3, 3-9-5, 5-9-3, 5-9-4, 4-9-5, 5-9-5, 4-11-4, 4-10-5 and 5-10-4.

For oligonucleotides of various lengths, the effect of certain chemistries was tested. Certain chemistry modifications were shown to be beneficial for a SNP targeting antisense oligonucleotide. In certain embodiments, each nucleoside of each wing of the modified antisense oligonucleotide has a 2′-MOE modification. In certain embodiments, each nucleoside of each wing of the modified antisense oligonucleotide has a high affinity modification. In certain embodiments, the antisense oligonucleotide is a mixed wing gapmer. In such embodiment, the modifications and combination of modifications at the 3′ wing and at the 5′ wing may be the same or they may be different. In certain embodiments, the antisense oligonucleotide has one or more 2′-MOE modifications in the wings and/or one or more high affinity modifications in the wings. In certain embodiments, the high affinity modification is a cEt modification. In certain embodiments, the antisense oligonucleotide has a high affinity modification at positions 2, 3, 13, and 14 of the antisense oligonucleotide (counting from the 5′ terminus). In certain embodiments, the antisense oligonucleotide has one, two, three, or four high affinity modifications in at least one of the wings. In certain embodiments, the antisense oligonucleotide has one, two, three, or four high affinity modifications in each of the 5′ and 3′ wings independently. In certain embodiments, the antisense oligonucleotide has a high affinity modification at positions 2 and 3 in one or both of the 5′ and 3′ wings (counting from the 5′ terminus of the 5′ wing and the 3′ terminus of the 3′wing). In certain embodiments, the antisense oligonucleotide has a high affinity modification at positions 2, 3 and 4 in one or both of the 5′ and 3′ wings (counting from the 5′ terminus of the 5′ wing and the 3′ terminus of the 3′wing,). In certain embodiments, the antisense oligonucleotide has a high affinity modification at positions 1 of the 5′ and/or 3′ wings (counting from the 5′ terminus of the 5′ wing and the 3′ terminus of the 3′wing,). In certain embodiments, the antisense oligonucleotide has a high affinity modification at positions 1 of the 5′ and 3′ wings (counting from the 5′ terminus of the 5′ wing and the 3′ terminus of the 3′wing,) and at least one other position in the wing. In certain embodiments, the antisense oligonucleotide has alternating 2′-MOE and high affinity modification in at least one of the 5′ and 3′wings.

In certain embodiments, the compound comprises an antisense oligonucleotide incorporating one or more of the design rules provided above.

In certain embodiments, the compound comprises a modified antisense oligonucleotide consisting of 12 to 30 linked nucleosides, fully complementary to a single nucleotide polymorphism site, wherein the modified antisense oligonucleotide comprises a wing-gap-wing motif, wherein the single nucleotide polymorphism aligns with any one of positions 6-15 beginning from the 5′ terminus of the antisense oligonucleotide or positions 1-9 beginning from the 5′ end of the gap of the modified antisense oligonucleotide; and wherein each nucleoside of each wing has a modified sugar or sugar surrogate. In certain embodiments the single nucleotide polymorphism site contains a differentiating polymorphism. In certain embodiments, the single nucleotide polymorphism site is on a mutant allele. In certain embodiments, the mutant allele is associated with disease. In certain embodiments, the wing-gap-wing motif is any one of the group consisting of 4-7-4, 5-8-6, 6-8-5, 6-7-6, 5-7-5. 6-8-5, 5-8-6, 3-9-4, 4-9-3, 2-9-6, 6,9,2,3-9-3, 3-9-5,5-9-3, 5-9-4, 4-9-5, 5-9-5, 4-11-4,4-10-5 and 5-10-4.

In certain embodiments, the compound comprises a modified antisense oligonucleotide consisting of 12 to 20 linked nucleosides, fully complementary to a single nucleotide polymorphism site, wherein the modified antisense oligonucleotide comprises a wing-gap-wing motif, wherein the single nucleotide polymorphism aligns with any one of positions 6-15 beginning from the 5′ terminus of the antisense oligonucleotide or positions 1-9 beginning from the 5′ end of the gap of the modified antisense oligonucleotide; and wherein each nucleoside of each wing has a modified sugar or sugar surrogate. In certain embodiments, the wing-gap-wing motif is any one of the group consisting of 4-7-4, 5-8-6, 6-8-5, 6-7-6, 5-7-5. 6-8-5, 5-8-6, 3-9-4, 4-9-3, 2-9-6, 6,9,2,3-9-3, 3-9-5,5-9-3, 5-9-4, 4-9-5, 5-9-5, 4-11-4,4-10-5 and 5-10-4.

In certain embodiments, the compound comprises a modified antisense oligonucleotide consisting of 12 to 20 linked nucleosides, fully complementary to a single nucleotide polymorphism site, wherein the modified antisense oligonucleotide comprises a wing-gap-wing motif, wherein the single nucleotide polymorphism aligns with any one of positions 8-14 beginning from the 5′ terminus of the antisense oligonucleotide or positions 1-9 beginning from the 5′ end of the gap of the modified antisense oligonucleotide; and wherein each nucleoside of each wing has a modified sugar or sugar surrogate. In certain embodiments, the wing-gap-wing motif is any one of the group consisting of 4-7-4, 5-8-6, 6-8-5, 6-7-6, 5-7-5. 6-8-5, 5-8-6, 3-9-4, 4-9-3, 2-9-6, 6,9,2,3-9-3, 3-9-5,5-9-3, 5-9-4, 4-9-5, 5-9-5, 4-11-4,4-10-5 and 5-10-4.

In certain embodiments, the compound comprises a modified antisense oligonucleotide consisting of 12 to 20 linked nucleosides, fully complementary to a single nucleotide polymorphism site, wherein the modified antisense oligonucleotide comprises a wing-gap-wing motif, wherein the single nucleotide polymorphism aligns with any one of positions 8-14 beginning from the 5′ terminus of the antisense oligonucleotide or positions 4-7 beginning from the 5′ end of the gap of the modified antisense oligonucleotide; and wherein each nucleoside of each wing has a modified sugar or sugar surrogate. In certain embodiments, the wing-gap-wing motif is any one of the group consisting of 4-7-4, 5-8-6, 6-8-5, 6-7-6, 5-7-5. 6-8-5, 5-8-6, 3-9-4, 4-9-3, 2-9-6, 6,9,2,3-9-3, 3-9-5,5-9-3, 5-9-4, 4-9-5, 5-9-5, 4-11-4,4-10-5 and 5-10-4.

In certain embodiments, the compound comprises a modified antisense oligonucleotide consisting of 12 to 20 linked nucleosides, fully complementary to a single nucleotide polymorphism site, wherein the modified antisense oligonucleotide comprises a wing-gap-wing motif, wherein the single nucleotide polymorphism aligns with any one of positions 8-10 beginning from the 5′ terminus of the antisense oligonucleotide or positions 4-6 beginning from the 5′ end of the gap of the modified antisense oligonucleotide; and wherein each nucleoside of each wing has a modified sugar or sugar surrogate. In certain embodiments, the wing-gap-wing motif is any one of the group consisting of 4-7-4, 5-8-6, 6-8-5, 6-7-6, 5-7-5. 6-8-5, 5-8-6, 3-9-4, 4-9-3, 2-9-6, 6,9,2,3-9-3, 3-9-5,5-9-3, 5-9-4, 4-9-5, 5-9-5, 4-11-4,4-10-5 and 5-10-4.

In certain embodiments, the compound comprises a modified antisense oligonucleotide consisting of 12 to 19 linked nucleosides, fully complementary to a single nucleotide polymorphism site, wherein the modified antisense oligonucleotide comprises a wing-gap-wing motif, wherein the single nucleotide polymorphism aligns with any one of positions 8-10 beginning from the 5′ terminus of the antisense oligonucleotide or positions 4-6 beginning from the 5′ end of the gap of the modified antisense oligonucleotide; and wherein each nucleoside of each wing has a modified sugar or sugar surrogate. In certain embodiments, the wing-gap-wing motif is any one of the group consisting of 4-7-4, 5-8-6, 6-8-5, 6-7-6, 5-7-5. 6-8-5, 5-8-6, 3-9-4, 4-9-3, 2-9-6, 6,9,2,3-9-3, 3-9-5,5-9-3, 5-9-4, 4-9-5, 5-9-5, 4-11-4,4-10-5 and 5-10-4.

In certain embodiments, the compound comprises a modified antisense oligonucleotide consisting of 13 to 19 linked nucleosides, fully complementary to a single nucleotide polymorphism site, wherein the modified antisense oligonucleotide comprises a wing-gap-wing motif, wherein the single nucleotide polymorphism aligns with any one of positions 8-10 beginning from the 5′ terminus of the antisense oligonucleotide or positions 4-6 beginning from the 5′ end of the gap of the modified antisense oligonucleotide; and wherein each nucleoside of each wing has a modified sugar or sugar surrogate. In certain embodiments, the wing-gap-wing motif is any one of the group consisting of 4-7-4, 5-8-6, 6-8-5, 6-7-6, 5-7-5. 6-8-5, 5-8-6, 3-9-4, 4-9-3, 2-9-6, 6,9,2,3-9-3, 3-9-5,5-9-3, 5-9-4, 4-9-5, 5-9-5, 4-11-4,4-10-5 and 5-10-4.

In certain embodiments, the compound comprises a modified antisense oligonucleotide consisting of 14 to 19 linked nucleosides, fully complementary to a single nucleotide polymorphism site, wherein the modified antisense oligonucleotide comprises a wing-gap-wing motif, wherein the single nucleotide polymorphism aligns with any one of positions 8-10 beginning from the 5′ terminus of the antisense oligonucleotide or positions 4-6 beginning from the 5′ end of the gap of the modified antisense oligonucleotide; and wherein each nucleoside of each wing has a modified sugar or sugar surrogate. In certain embodiments, the wing-gap-wing motif is any one of the group consisting of 4-7-4, 5-8-6, 6-8-5, 6-7-6, 5-7-5. 6-8-5, 5-8-6, 3-9-4, 4-9-3, 2-9-6, 6,9,2,3-9-3, 3-9-5,5-9-3, 5-9-4, 4-9-5, 5-9-5, 4-11-4,4-10-5 and 5-10-4.

In certain embodiments, the compound comprises a modified antisense oligonucleotide consisting of 15 to 19 linked nucleosides, fully complementary to a single nucleotide polymorphism site, wherein the modified antisense oligonucleotide comprises a wing-gap-wing motif, wherein the single nucleotide polymorphism aligns with any one of positions 6-15 beginning from the 5′ terminus of the antisense oligonucleotide or positions 1-9 beginning from the 5′ end of the gap of the modified antisense oligonucleotide; and wherein each nucleoside of each wing has a modified sugar or sugar surrogate. In certain embodiments, the wing-gap-wing motif is any one of the group consisting of 4-7-4, 5-8-6, 6-8-5, 6-7-6, 5-7-5. 6-8-5, 5-8-6, 3-9-4, 4-9-3, 2-9-6, 6,9,2,3-9-3, 3-9-5,5-9-3, 5-9-4, 4-9-5, 5-9-5, 4-11-4,4-10-5 and 5-10-4.

In certain embodiments, the compound comprises a modified antisense oligonucleotide consisting of 15 to 19 linked nucleosides, fully complementary to a single nucleotide polymorphism site, wherein the modified antisense oligonucleotide comprises a wing-gap-wing motif, wherein the single nucleotide polymorphism aligns with any one of positions 8-10 beginning from the 5′ terminus of the antisense oligonucleotide or positions 4-6 beginning from the 5′ end of the gap of the modified antisense oligonucleotide; and wherein each nucleoside of each wing has a modified sugar or sugar surrogate. In certain embodiments, the wing-gap-wing motif is any one of the group consisting of 4-7-4, 5-8-6, 6-8-5, 6-7-6, 5-7-5. 6-8-5, 5-8-6, 3-9-4, 4-9-3, 2-9-6, 6,9,2,3-9-3, 3-9-5,5-9-3, 5-9-4, 4-9-5, 5-9-5, 4-11-4,4-10-5 and 5-10-4.

In certain embodiments, the compound comprises a modified antisense oligonucleotide consisting of 15 to 19 linked nucleosides, fully complementary to a single nucleotide polymorphism site, wherein the modified antisense oligonucleotide comprises a wing-gap-wing motif, wherein position 6, 8, 9, 10, 11, or 14 beginning from the 5′ terminus of the modified antisense oligonucleotide aligns with the single nucleotide polymorphism; and wherein each nucleoside of each wing segment modified sugar or sugar surrogate. In certain embodiments, the wing-gap-wing motif is any one of the group consisting of 2-9-6, 3-9-3, 3-9-5, 4-9-5, 4-11-4, and 5-10-4.

In certain embodiments, the compound comprises a modified antisense oligonucleotide consisting of 15 to 19 linked nucleosides, fully complementary to a single nucleotide polymorphism site, wherein the modified antisense oligonucleotide comprises a wing-gap-wing motif, wherein position 1, 4, 5, 6, 7, or 9 of the gap segment aligns with the single nucleotide polymorphism; and wherein each nucleoside of each wing segment has a modified sugar or sugar surrogate. In certain embodiments, the wing-gap-wing motif is any one of the group consisting of 2-9-6, 3-9-3, 3-9-5, 4-9-5, 4-11-4, and 5-10-4.

In certain embodiments, the compound comprises a modified antisense oligonucleotide consisting of 15 to 19 linked nucleosides, fully complementary to a single nucleotide polymorphism site, wherein the modified antisense oligonucleotide comprises a wing-gap-wing motif, wherein position 6, 7, 8, 9, 10, 11, or 12 of the modified antisense oligonucleotide aligns with the single nucleotide polymorphism; and positions 2 and 3 of the 5′ and 3′ wing segments comprise a 4′-CH(CH₃)—O-2′ bridge. In certain embodiments, the wing-gap-wing motif is any one of the group consisting of 2-9-6, 3-9-3, 3-9-5, 4-9-5, 4-11-4, and 5-10-4.

In certain embodiments, the compound comprises a modified antisense oligonucleotide consisting of 15 to 19 linked nucleosides and fully complementary to a single nucleotide polymorphism site, wherein the modified antisense oligonucleotide comprises a wing-gap-wing motif, wherein position 3, 4, 5, 6, 7, 8 or 9 of the gap segment aligns with the single nucleotide polymorphism; and positions 2 and 3 of the 5′ and 3′ wing segments comprise a 4′-CH(CH₃)—O-2′ bridge. In certain embodiments, the wing-gap-wing motif is any one of the group consisting of 2-9-6, 3-9-3, 3-9-5, 4-9-5, 4-11-4, and 5-10-4.

A compound comprising a modified antisense oligonucleotide consisting of 15 to 19 linked nucleosides and fully complementary to a single nucleotide polymorphism site, wherein the modified antisense oligonucleotide comprises a wing-gap-wing motif, wherein position 6, 7, 8, 9, 10, 11, or 12 of the modified antisense oligonucleotide aligns with the single nucleotide polymorphism; and positions 2, 3, 13, and 14 of the antisense oligonucleotide comprise a 4′-CH(CH₃)—O-2′ bridge. In certain embodiments, the wing-gap-wing motif is any one of the group consisting of 2-9-6, 3-9-3, 3-9-5, 4-9-5, 4-11-4, and 5-10-4.

A compound comprising a modified antisense oligonucleotide consisting of 15 to 19 linked nucleosides and fully complementary to a single nucleotide polymorphism site, wherein the modified antisense oligonucleotide comprises a wing-gap-wing motif, wherein position 3, 4, 5, 6, 7, 8, or 9 of the gap segment aligns with the single nucleotide polymorphism; and positions 2, 3, 13, and 14 of the antisense oligonucleotide comprise a 4′-CH(CH₃)—O-2′ bridge. In certain embodiments, the wing-gap-wing motif is any one of the group consisting of 2-9-6, 3-9-3, 3-9-5, 4-9-5, 4-11-4, and 5-10-4.

In certain embodiments, the compound comprise a modified antisense oligonucleotide consisting of 17 to 19 linked nucleosides, fully complementary to a single nucleotide polymorphism site, wherein the modified antisense oligonucleotide comprises a wing-gap-wing motif, wherein position 8, 9, or 10 of the modified antisense oligonucleotide aligns with the single nucleotide polymorphism; and wherein each nucleoside of each wing segment comprises a 2′-O-methoxyethyl sugar. In certain embodiments, the wing-gap-wing motif is any one of the group consisting of 2-9-6, 3-9-3, 3-9-5, 4-9-5, 4-11-4, and 5-10-4.

In certain embodiments, the compound comprises a modified antisense oligonucleotide consisting of 17 to 19 linked nucleosides, fully complementary to a single nucleotide polymorphism site, wherein the modified antisense oligonucleotide comprises a wing-gap-wing motif, wherein position 5, 6, or 7 of the gap segment aligns with the single nucleotide polymorphism; and wherein each nucleoside of each wing segment comprises a 2′-O-methoxyethyl sugar. In certain embodiments, the wing-gap-wing motif is any one of the group consisting of 2-9-6, 3-9-3, 3-9-5, 4-9-5, 4-11-4, and 5-10-4.

In certain embodiments, the compound comprises a modified antisense oligonucleotide consisting of 17 to 19 linked nucleosides, fully complementary to a single nucleotide polymorphism site, wherein the modified antisense oligonucleotide comprises a wing-gap-wing motif, wherein position 8, 9, or 10 of the modified antisense oligonucleotide aligns with the single nucleotide polymorphism; and positions 2 and 3 of the 5′ and 3′ wing segments comprise a 4′-CH(CH₃)—O-2′ bridge. In certain embodiments, the wing-gap-wing motif is any one of the group consisting of 2-9-6, 3-9-3, 3-9-5, 4-9-5, 4-11-4, and 5-10-4.

In certain embodiments, the compound comprises a modified antisense oligonucleotide consisting of 17 to 19 linked nucleosides and fully complementary to a single nucleotide polymorphism site, wherein the modified antisense oligonucleotide comprises a wing-gap-wing motif, wherein position 5, 6, or 7 of the gap segment aligns with the single nucleotide polymorphism; and positions 2 and 3 of the 5′ and 3′ wing segments comprise a 4′-CH(CH₃)—O-2′ bridge. In certain embodiments, the wing-gap-wing motif is any one of the group consisting of 2-9-6, 3-9-3, 3-9-5, 4-9-5, 4-11-4, and 5-10-4.

A compound comprising a modified antisense oligonucleotide consisting of 17 to 19 linked nucleosides and fully complementary to a single nucleotide polymorphism site, wherein the modified antisense oligonucleotide comprises a wing-gap-wing motif, wherein position 8, 9, or 10 of the modified oligonucleotide aligns with the single nucleotide polymorphism; and positions 2, 3, 13, and 14 of the antisense oligonucleotide comprise a 4′-CH(CH₃)—O-2′ bridge. In certain embodiments, the wing-gap-wing motif is any one of the group consisting of 2-9-6, 3-9-3, 3-9-5, 4-9-5, 4-11-4, and 5-10-4.

A compound comprising a modified antisense oligonucleotide consisting of 17 to 19 linked nucleosides and fully complementary to a single nucleotide polymorphism site, wherein the modified antisense oligonucleotide comprises a wing-gap-wing motif, wherein position 5, 6, or 7 of the gap segment aligns with the single nucleotide polymorphism; and positions 2, 3, 13, and 14 of the antisense oligonucleotide comprise a 4′-CH(CH₃)—O-2′ bridge. In certain embodiments, the wing-gap-wing motif is any one of the group consisting of 2-9-6, 3-9-3, 3-9-5, 4-9-5, 4-11-4, and 5-10-4.

In a certain embodiment, the antisense oligonucleotide is 11 to 20 linked nucleosides in length and has, independently, 2 to 5 linked nucleosides in the 5′ and 3′ wings and 7 to 11 linked nucleosides in the gap. The SNP is complementary to position 5, 6, 7, 8, 9, 10, 11, 12, 13, or 14 of the antisense oligonucleotide (counting from the 5′ terminus of the antisense oligonucleotide) or position 1, 2, 3, 4, 5, 6, 7, 8, 9, or 10 counting from the 5′ terminus of the gap segment.

In a certain embodiment, the antisense oligonucleotide is 15 to 19 linked nucleosides in length and has, independently, 2 to 5 linked nucleosides in the 5′ and 3′ wings and 7 to 11 linked nucleosides in the gap. The SNP is complementary to position 6, 7, 8, 9, or 10 of the antisense oligonucleotide (counting from the 5′ terminus of the antisense oligonucleotide) or position 4, 5, 6, or 7 counting from the 5′ terminus of the gap segment.

In a certain embodiment, the antisense oligonucleotide is 17 linked nucleosides in length and has, independently, 2 to 5 linked nucleosides in the 5′ and 3′ wing segments and 9 to 11 linked nucleosides in the gap segment. The SNP is complementary to position 8, 9, or 10 of the antisense oligonucleotide (counting from the 5′ terminus of the antisense oligonucleotide) or position 5, 6, or 7 (counting from the 5′ terminus of the gap segment).

In a certain embodiment, the antisense oligonucleotide is 18 linked nucleosides in length and has, independently, 2 to 5 linked nucleosides in the 5′ and 3′ wing segments and 9 to 11 linked nucleosides in the gap segment. The SNP is complementary to position 8, 9, or 10 of the antisense oligonucleotide (counting from the 5′ terminus of the antisense oligonucleotide) or position 5, 6, or 7 (counting from the 5′ terminus of the gap segment).

In a certain embodiment, the antisense oligonucleotide is 19 linked nucleosides in length and has, independently, 2 to 5 linked nucleosides in the 5′ and 3′ wing segments and 9 to 11 linked nucleosides in the gap segment. The SNP is complementary to position 8, 9, or 10 of the antisense oligonucleotide (counting from the 5′ terminus of the antisense oligonucleotide) or position 5, 6, or 7 (counting from the 5′ terminus of the gap segment).

In certain embodiments, the invention provides methods of treating an individual comprising administering one or more pharmaceutical compositions described herein. In certain embodiments, the individual has an allelic variant associated with a disease or disorder. The pharmaceutical compositions provided herein preferentially target a SNP. In certain embodiments, the SNP is a differentiating polymorphism.

Methods have been described for determining whether a SNP is specific to a disease associated allele and more specifically whether a SNP variant of an allele of a heterozygous patient is on the same allele as a disease-causing mutation that is at a remote region of the gene's mRNA (WO 2008/147930 and WO 2008/143774).

Diseases associated with SNPs have been described for certain genes. In certain embodiments, the gene and associated disease are any of the following: APP gene encoding amyloid precursor protein involved in Alzheimer's disease (Gene, 371:68, 2006); the PrP gene encoding prion protein involved in Creutzfeldt-Jakob disease and in fatal familial insomnia (Nat. Med. 1997, 3:1009); GFAP gene encoding glial fibrillary acidic protein involved in Alexander disease (J. Neurosci. 2006, 26:111623); alpha-synuclein gene encoding alpha-synuclein protein involved in Parkinson's disease (J. Clin. Invest. 2003, 111:145); SOD-1 gene encoding the SOD-1 protein involved in amyotrophic lateral sclerosis (Science 1998, 281:1851); atrophin-1 gene encoding atrophin-1 protein involved in dentato-rubral and pallido-luysian atrophy (DRPA) (Trends Mol. Med. 2001, 7:479); SCA1 gene encoding ataxin-1 protein involved in spino-cerebellar ataxia-1 (SCA1) (Protein Sci. 2003, 12:953); PLP gene encoding proteolipid protein involved in Pelizaeus-Merzbacher disease (NeuroMol Med. 2007, 4:73); DYTI gene encoding torsinA protein involved in Torsion dystonia (Brain Res. 2000, 877:379); and alpha-B crystalline gene encoding alpha-B crystalline protein involved in protein aggregation diseases, including cardiomyopathy (Cell 2007, 130:427); alpha1-antitrypsin gene encoding alpha1-antitrypsin protein involved in chronic obstructive pulmonary disease (COPD), liver disease and hepatocellular carcinoma (New Engl J Med. 2002, 346:45); Ltk gene encoding leukocyte tyrosine kinase protein involved in systemic lupus erythematosus (Hum. Mol. Gen. 2004, 13:171); PCSK9 gene encoding PCSK9 protein involved in hypercholesterolemia (Hum Mutat. 2009, 30:520); prolactin receptor gene encoding prolactin receptor protein involved in breast tumors (Proc. Natl. Assoc. Sci. 2008, 105:4533); CCL5 gene encoding the chemokine CCL5 involved in COPD and asthma (Eur. Respir. J. 2008, 32:327); PTPN22 gene encoding PTPN22 protein involved in Type 1 diabetes, Rheumatoid arthritis, Graves disease, and SLE (Proc. Natl. Assoc. Sci. 2007, 104:19767); androgen receptor gene encoding the androgen receptor protein involved in spinal and bulbar muscular atrophy or Kennedy's disease (J Steroid Biochem. Mol. Biol. 2008, 108:245); CHMP4B gene encoding chromatin modifying protein-4B involved in progressive childhood posterior subcapsular cataracts (Am. J. Hum. Genet 2007, 81:596); FXR/NR1H4 gene encoding Farnesoid X receptor protein involved in cholesterol gallstone disease, arthrosclerosis and diabetes (Mol. Endocrinol. 2007, 21:1769); ABCA1 gene encoding ABCA1 protein involved in cardiovascular disease (Transl. Res. 2007, 149:205); CaSR gene encoding the calcium sensing receptor protein involved in primary hypercalciuria (Kidney Int. 2007, 71:1155); alpha-globin gene encoding alpha-globin protein involved in alpha-thallasemia (Science 2006, 312:1215); httlpr gene encoding HTTLPR protein involved in obsessive compulsive disorder (Am. J. Hum. Genet. 2006, 78:815); AVP gene encoding arginine vasopressin protein in stress-related disorders such as anxiety disorders and comorbid depression (CNS Neurol. Disord. Drug Targets 2006, 5:167); GNAS gene encoding G proteins involved in congenital visual defects, hypertension, metabolic syndrome (Trends Pharmacol. Sci. 2006, 27:260); APAF1 gene encoding APAF1 protein involved in a predisposition to major depression (Mol. Psychiatry 2006, 11:76); TGF-beta1 gene encoding TGF-beta1 protein involved in breast cancer and prostate cancer (Cancer Epidemiol. Biomarkers Prev. 2004, 13:759); AChR gene encoding acetylcholine receptor involved in congential myasthenic syndrome (Neurology 2004, 62:1090); P2Y12 gene encoding adenosine diphosphate (ADP) receptor protein involved in risk of peripheral arterial disease (Circulation 2003, 108:2971); LQT1 gene encoding LQT1 protein involved in atrial fibrillation (Cardiology 2003, 100:109); RET protooncogene encoding RET protein involved in sporadic pheochromocytoma (J. Clin. Endocrinol. Metab. 2003, 88:4911); filamin A gene encoding filamin A protein involved in various congenital malformations (Nat. Genet. 2003, 33:487); TARDBP gene encoding TDP-43 protein involved in amyotrophic lateral sclerosis (Hum. Mol. Gene.t 2010, 19:671); SCA3 gene encoding ataxin-3 protein involved in Machado-Joseph disease (PLOS One 2008, 3: e3341); SCA7 gene encoding ataxin-7 protein involved in spino-cerebellar ataxia-7 (PLOS One 2009, 4: e7232); HTT gene encoding huntingtin protein involved in Huntington's disease (Neurobiol Dis. 1996, 3:183); and the CA4 gene encoding carbonic anhydrase 4 protein, CRX gene encoding cone-rod homeobox transcription factor protein, FSCN2 gene encoding retinal fascin homolog 2 protein, IMPDH1 gene encoding inosine monophosphate dehydrogenase 1 protein, NR2E3 gene encoding nuclear receptor subfamily 2 group E3 protein, NRL gene encoding neural retina leucine zipper protein, PRPF3 (RP18) gene encoding pre-mRNA splicing factor 3 protein, PRPF8 (RP13) gene encoding pre-mRNA splicing factor 8 protein, PRPF31 (RP11) gene encoding pre-mRNA splicing factor 31 protein, RDS gene encoding peripherin 2 protein, ROM1 gene encoding rod outer membrane protein 1 protein, RHO gene encoding rhodopsin protein, RP1 gene encoding RP1 protein, RPGR gene encoding retinitis pigmentosa GTPase regulator protein, all of which are involved in Autosomal Dominant Retinitis Pigmentosa disease (Adv Exp Med Biol. 2008, 613:203)

In certain embodiments, the disease is a neurodegenerative disorder. In certain embodiments, the neurodegenerative disorder is Huntington's Disease. In certain embodiments, the targeted SNP is one or more of: rs6446723, rs3856973, rs2285086, rs363092, rs916171, rs6844859, rs7691627, rs4690073, rs2024115, rs11731237, rs362296, rs10015979, rs7659144, rs363096, rs362273, rs16843804, rs362271, rs362275, rs3121419, rs362272, rs3775061, rs34315806, rs363099, rs2298967, rs363088, rs363064, rs363102, rs2798235, rs363080, rs363072, rs363125, rs362303, rs362310, rs10488840, rs362325, rs35892913, rs363102, rs363096, rs11731237, rs10015979, rs363080, rs2798235, rs1936032, rs2276881, rs363070, rs35892913, rs12502045, rs6446723, rs7685686, rs3733217, rs6844859, rs362331, rs1143646, rs2285086, rs2298969, rs4690072, rs916171, rs3025849, rs7691627, rs4690073, rs3856973, rs363092, rs362310, rs362325, rs363144, rs362303, rs34315806, rs363099, rs363081, rs3775061, rs2024115, rs10488840, rs363125, rs362296, rs2298967, rs363088, rs363064, rs362275, rs3121419, rs3025849, rs363070, rs362273, rs362272, rs362306, rs362271, rs363072, rs16843804, rs7659144, rs363120, and rs12502045. In certain embodiments the compounds are ISIS460065, ISIS 459978, ISIS 460028, ISIS 460209, ISIS 460208, and ISIS 460206.

### Therapeutically Effective Dosages

In certain embodiments, administration of a therapeutically effective amount of an antisense compound targeted to the mutant huntingtin allele is accompanied by monitoring of expression of a gene product in an individual, to determine an individual's response to administration of the antisense compound. In certain embodiments, the gene product is huntingtin mRNA or protein. An individual's response to administration of the antisense compound is used by a physician to determine the amount and duration of therapeutic intervention.

In certain embodiments, administration of an antisense compound targeted to a mutant nucleic acid results in reduction of mRNA or protein expression by at least 15, 20, 25, 30, 35, 40, 45, 50, 55, 60, 65, 70, 75, 80, 85, 90, 95 or 99%, or a range defined by any two of these values. In certain embodiments, the mutant nucleic acid is huntingtin nucleic acid, the mRNA is huntingtin mRNA, and the protein is huntingtin protein.

In certain embodiments, pharmaceutical compositions comprising an antisense compound targeted to a mutant allele are used for the preparation of a medicament for treating a patient suffering or susceptible to any of Huntington's Disease, Alzheimer's Disease, Crutzfeldt-Jakob Disease, Fatal Familial Insomnia, Huntington's Disease, Alexander Disease, Parkinson's Disease, Amyotrophic Lateral Sclerosis (ALS), Dentato-Rubral and Pallido-Luysian Atrophy, Spino-Cerebellar Ataxia 1, Pelizaeus-Merzbacher Disease, Torsion Dystonia, Cardiomyopathy, Chronic Obstructive Pulmonary Disease (COPD), liver disease and hepatocellular carcinoma, SLE, Hypercholesterolemia, breast tumors, Asthma, Type 1 Diabetes, Rheumatoid Arthritis, Graves Disease, Spinal and Bulbar Muscular Atrophy, Kennedy's Disease, progressive childhood posterior subcapsular cataracts, Cholesterol Gallstone Disease, Arthrosclerosis, cardiovascular disease, primary hypercalciuria, alpha-thallasemia, OCD, stress-related disorders (including anxiety disorders and comorbid depression), congential visual defects, hypertension, metabolic syndrome, major depression, breast cancer, prostate cancer, congenital myasthenic syndrome, peripheral arterial syndrome, atrial fibrillation, sporadic pheochromocytoma, congenital malformations, NJD, SCA7, and autosomal dominant retinitis pigmentosa adRP.

### Certain Combination Therapies

In certain embodiments, one or more pharmaceutical compositions of the present invention are co-administered with one or more other pharmaceutical agents. In certain embodiments, such one or more other pharmaceutical agents are designed to treat the same disease, disorder, or condition as the one or more pharmaceutical compositions of the present invention. In certain embodiments, such one or more other pharmaceutical agents are designed to treat a different disease, disorder, or condition as the one or more pharmaceutical compositions of the present invention. In certain embodiments, such one or more other pharmaceutical agents are designed to treat an undesired side effect of one or more pharmaceutical compositions of the present invention. In certain embodiments, one or more pharmaceutical compositions of the present invention are co-administered with another pharmaceutical agent to treat an undesired effect of that other pharmaceutical agent. In certain embodiments, one or more pharmaceutical compositions of the present invention are co-administered with another pharmaceutical agent to produce a combinational effect. In certain embodiments, one or more pharmaceutical compositions of the present invention are co-administered with another pharmaceutical agent to produce a synergistic effect.

In certain embodiments, one or more pharmaceutical compositions of the present invention and one or more other pharmaceutical agents are administered at the same time. In certain embodiments, one or more pharmaceutical compositions of the present invention and one or more other pharmaceutical agents are administered at different times. In certain embodiments, one or more pharmaceutical compositions of the present invention and one or more other pharmaceutical agents are prepared together in a single formulation. In certain embodiments, one or more pharmaceutical compositions of the present invention and one or more other pharmaceutical agents are prepared separately.

### EXAMPLES

### Non-Limiting Disclosure and Incorporation by Reference

While certain compounds, compositions and methods described herein have been described with specificity in accordance with certain embodiments, the following examples serve only to illustrate the compounds described herein and are not intended to limit the same. Each of the patents, applications, printed publications, and other published documents mentioned or referred to in this specification are herein incorporated by reference in their entirety.

### Example 1: Single Nucleotide Polymorphisms (SNPs) in the Huntingtin (HTT) Gene Sequence

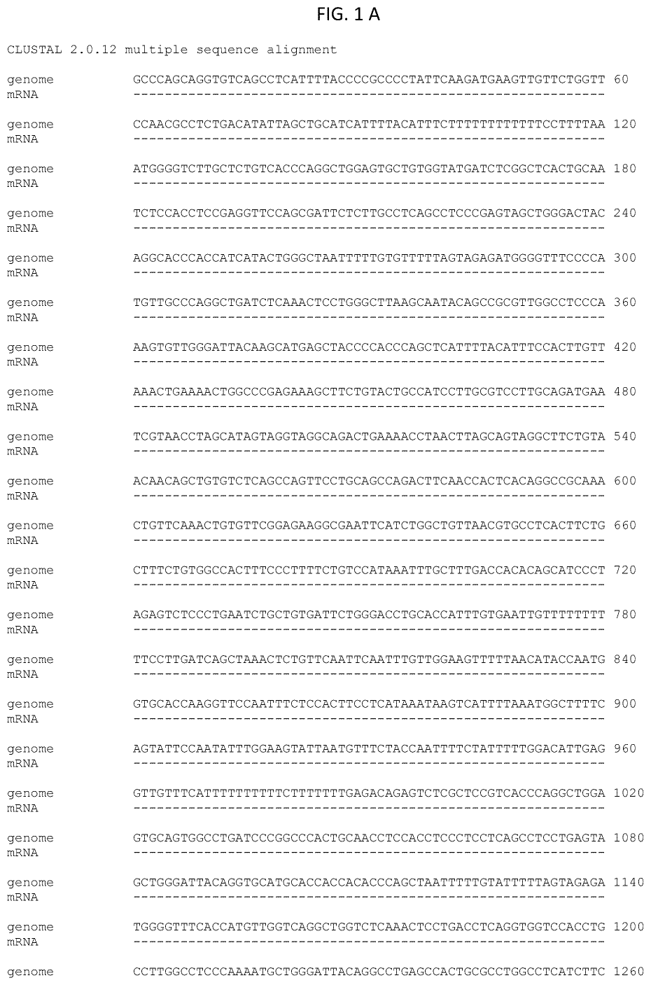

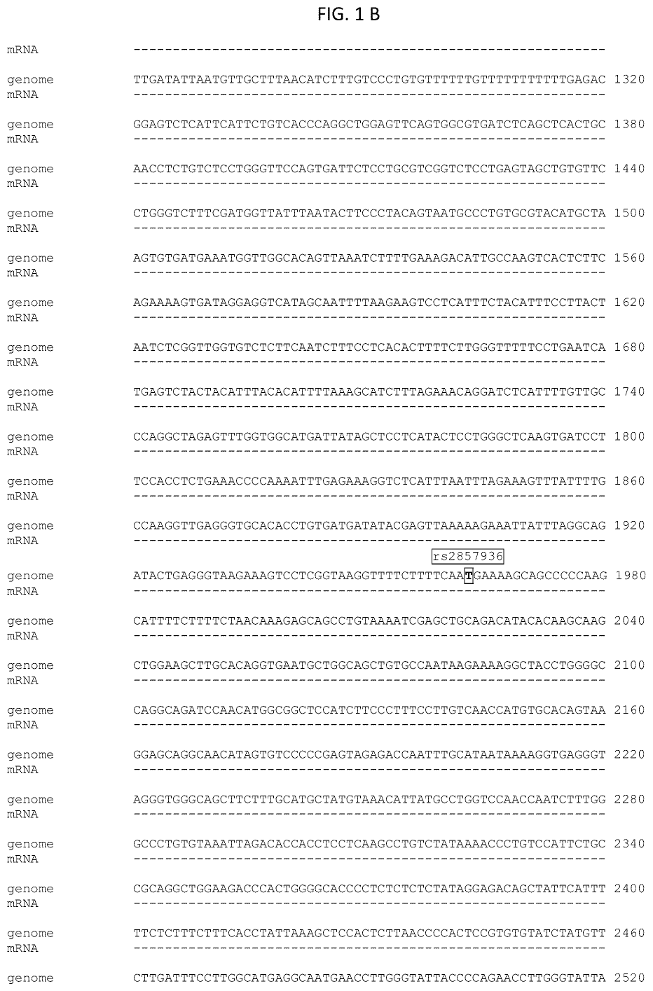

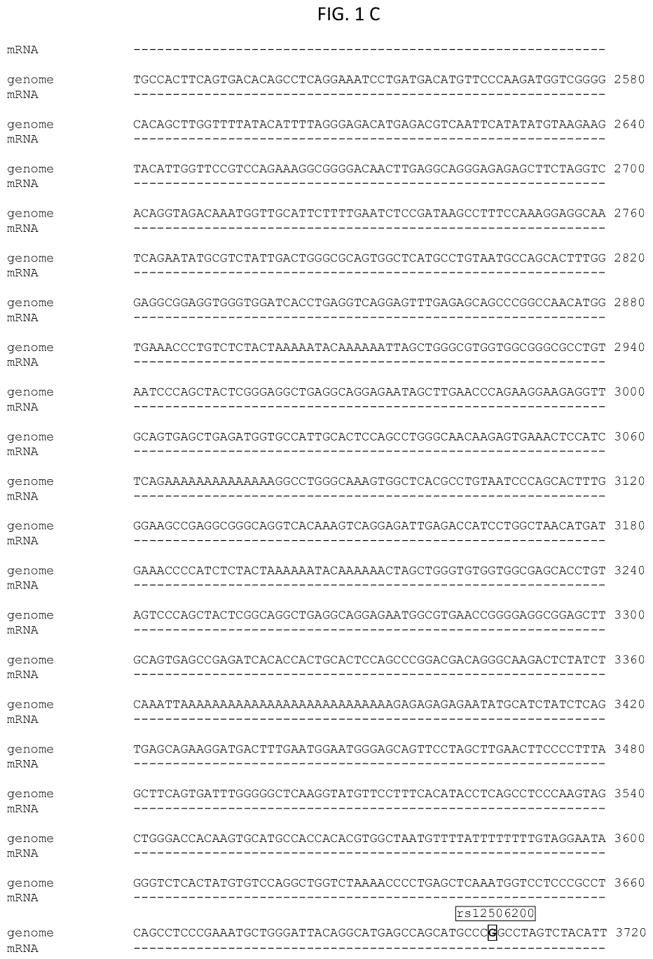

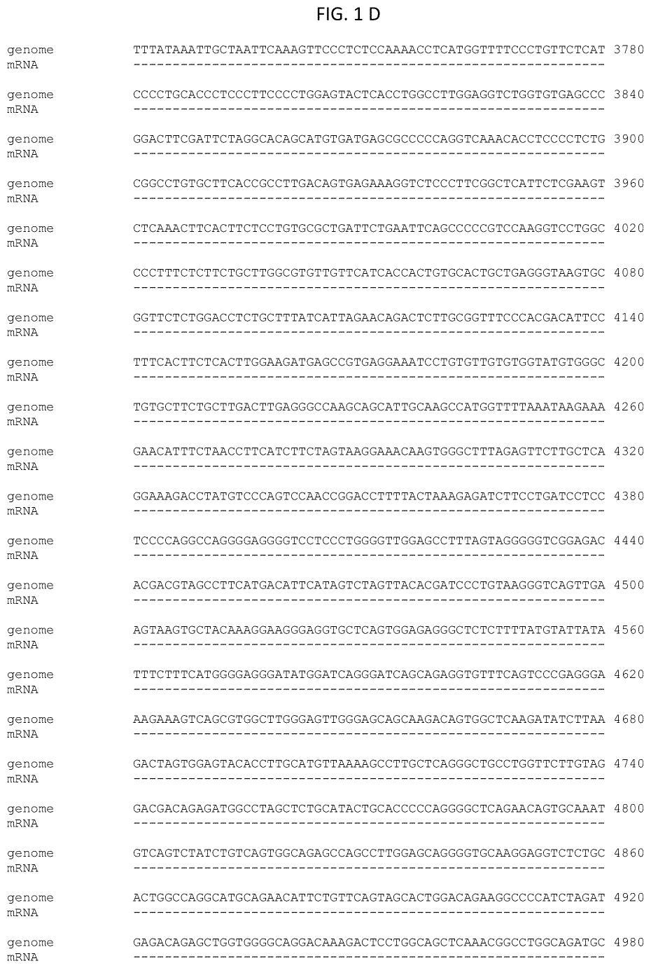

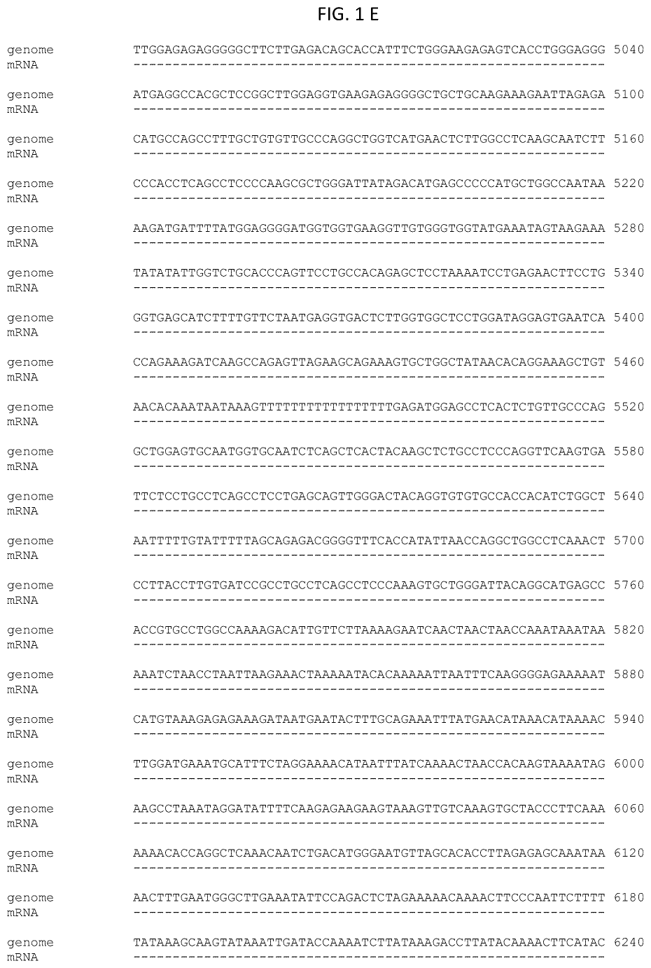

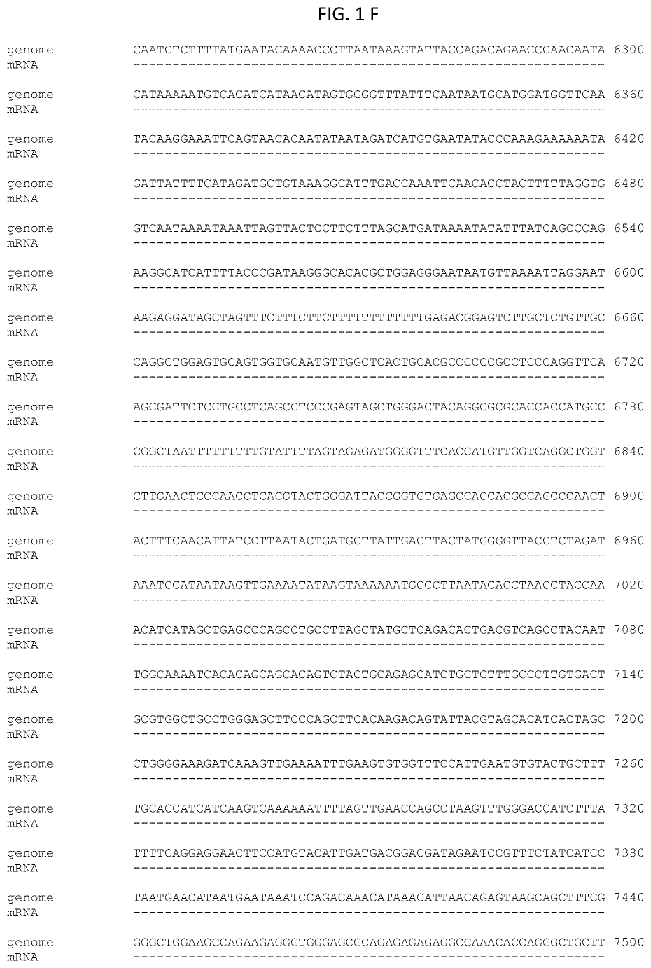

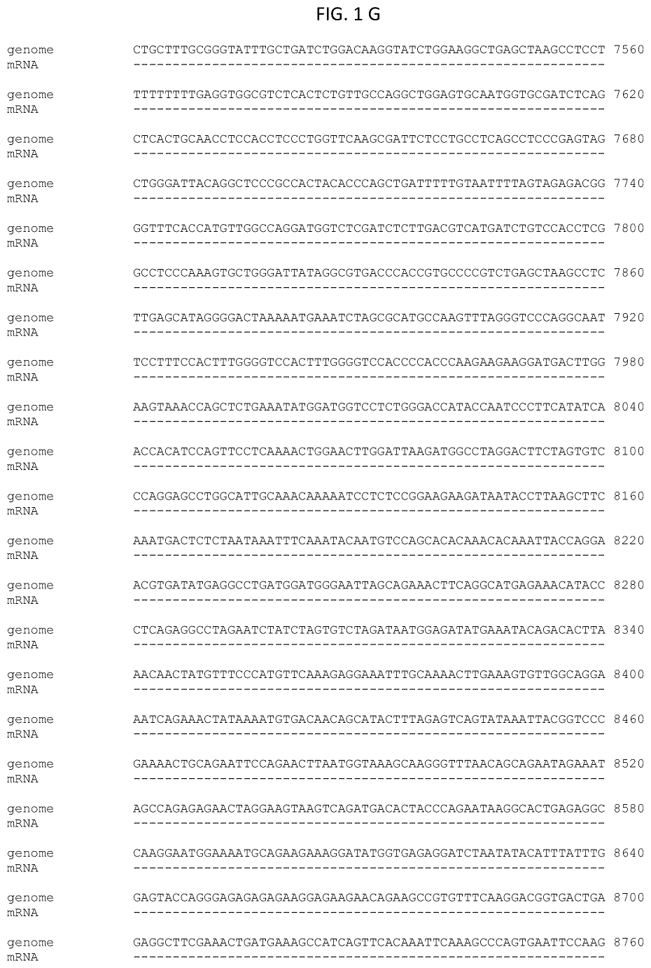

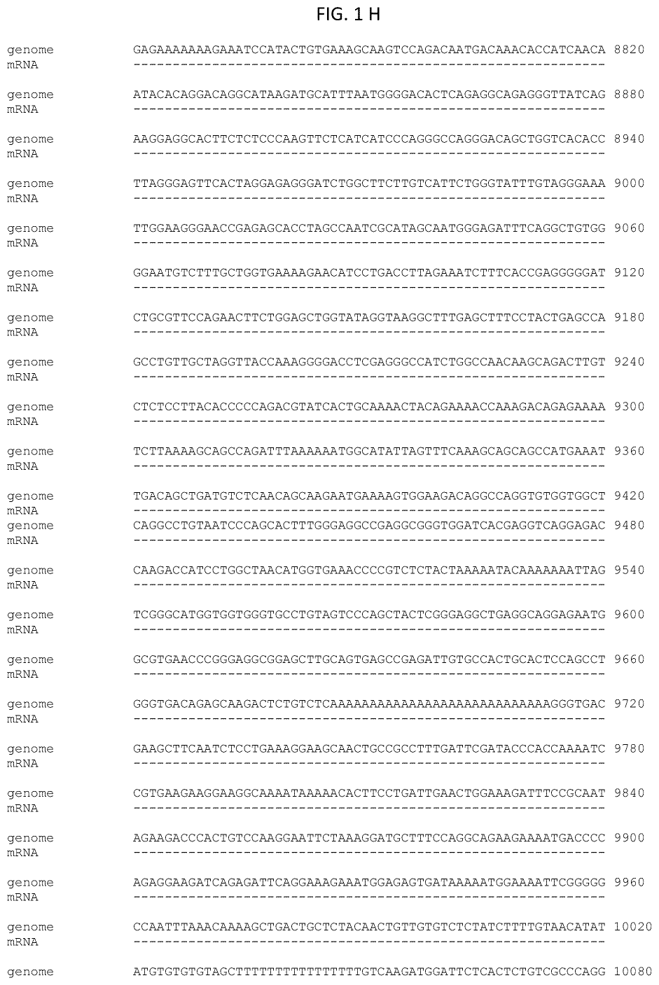

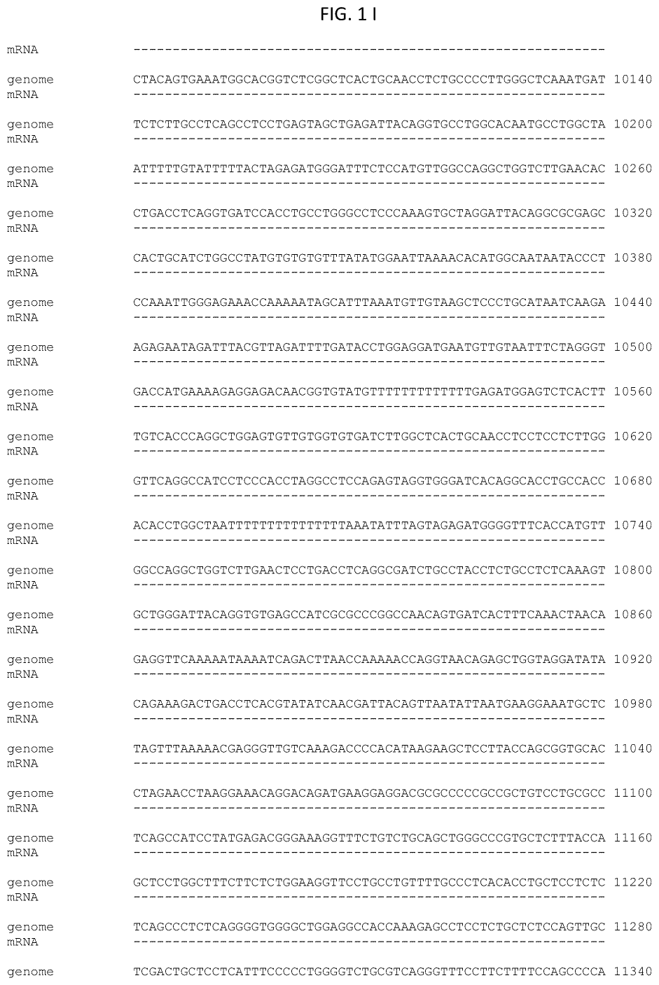

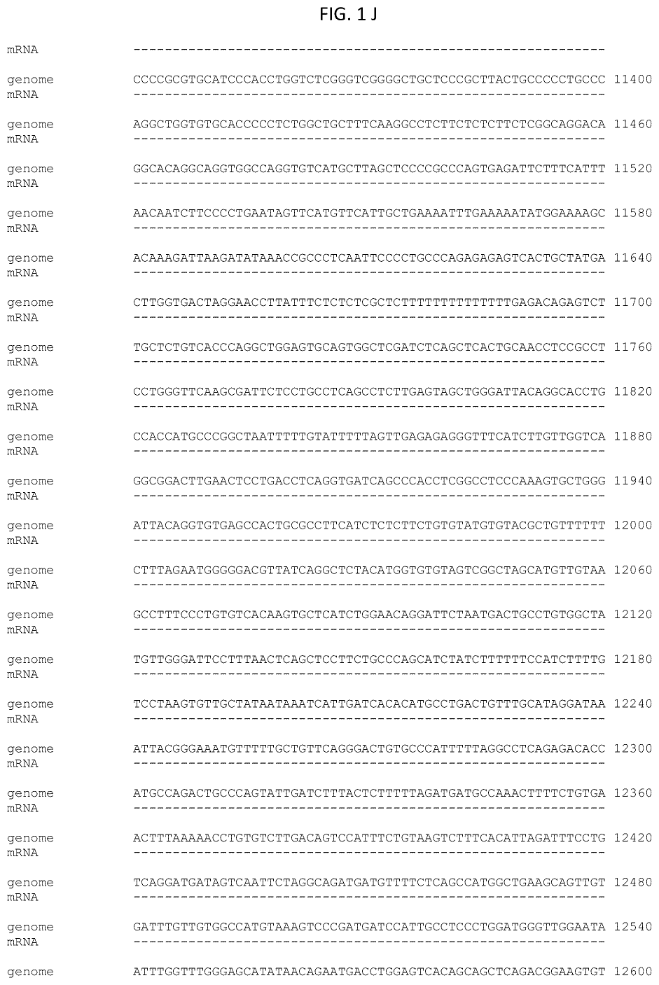

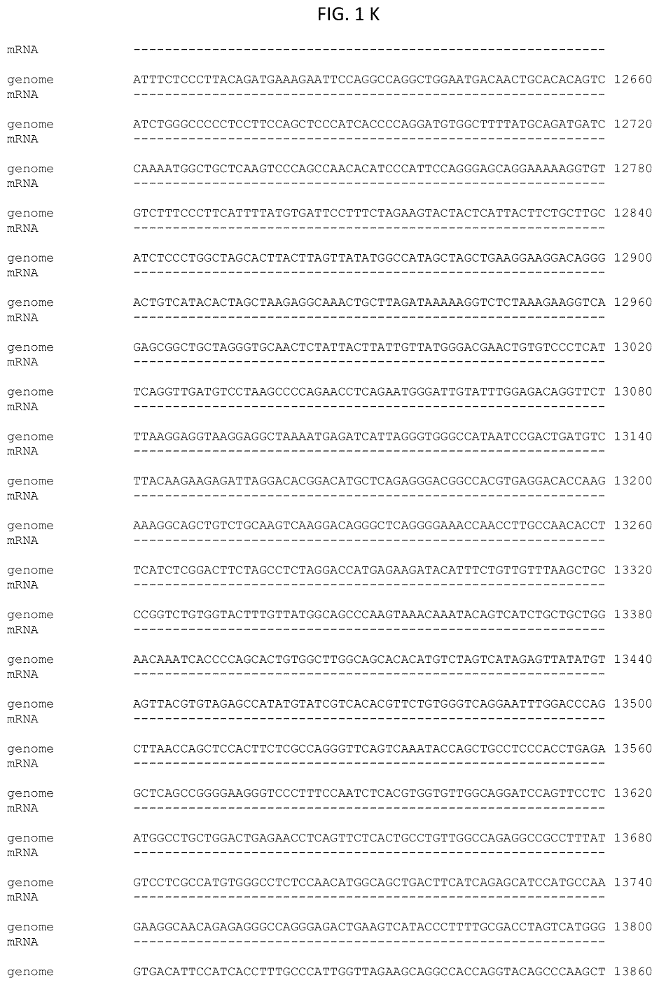

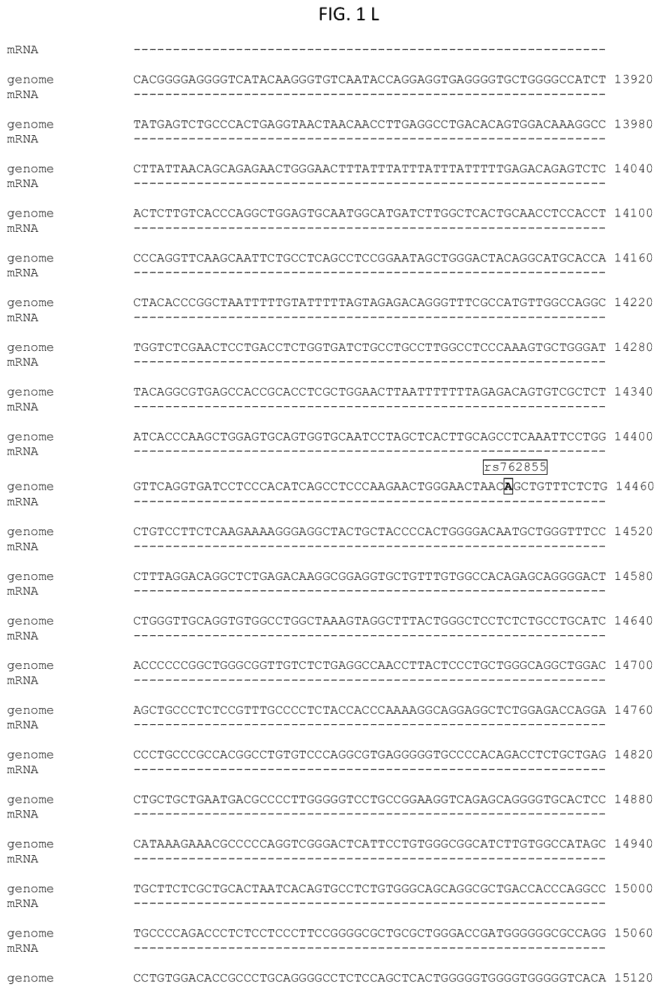

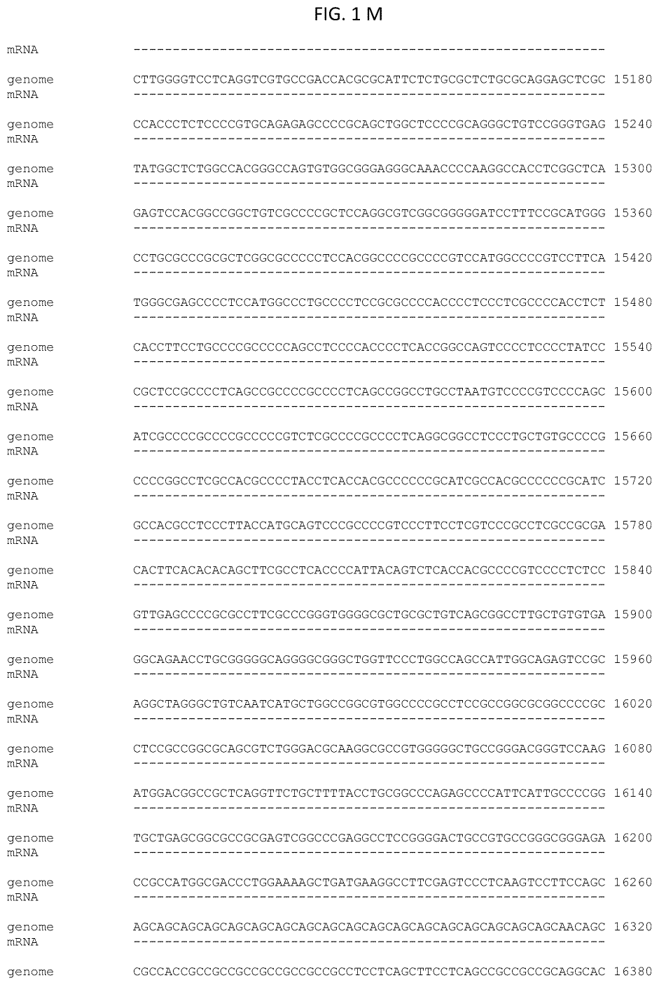

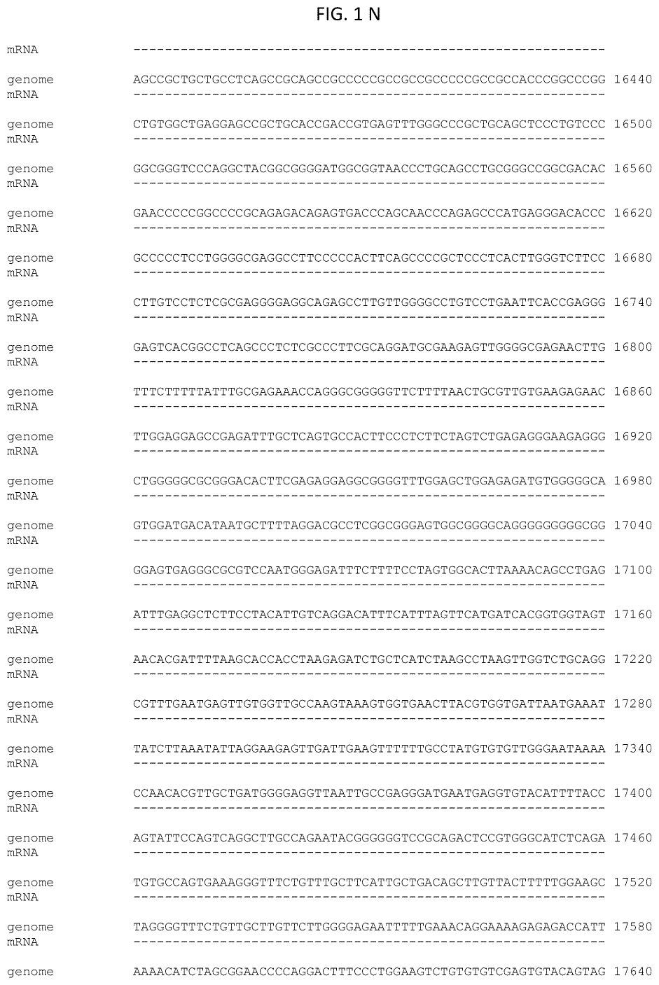

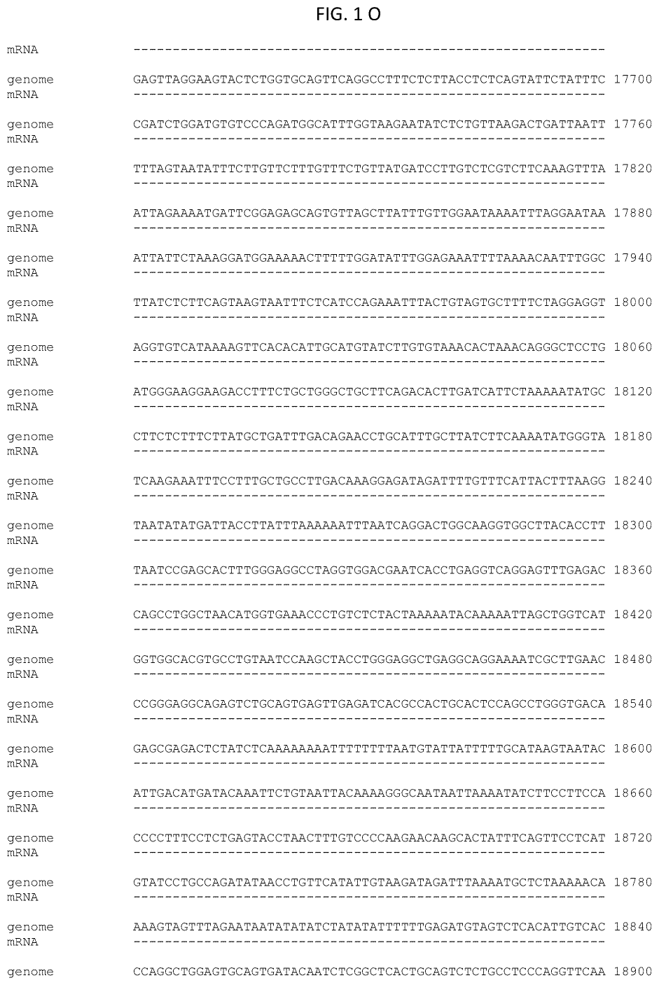

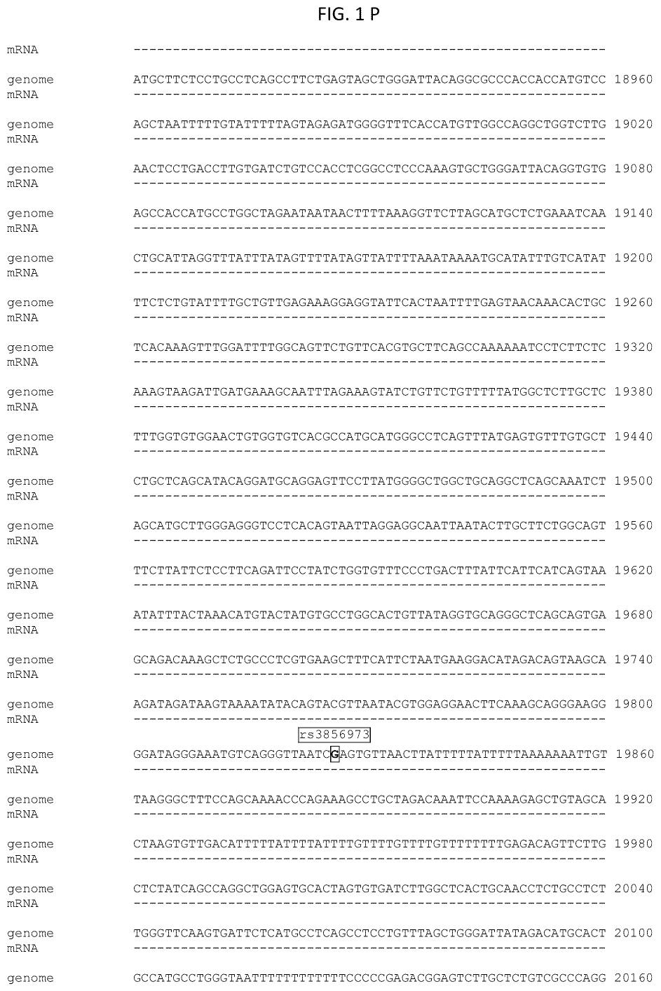

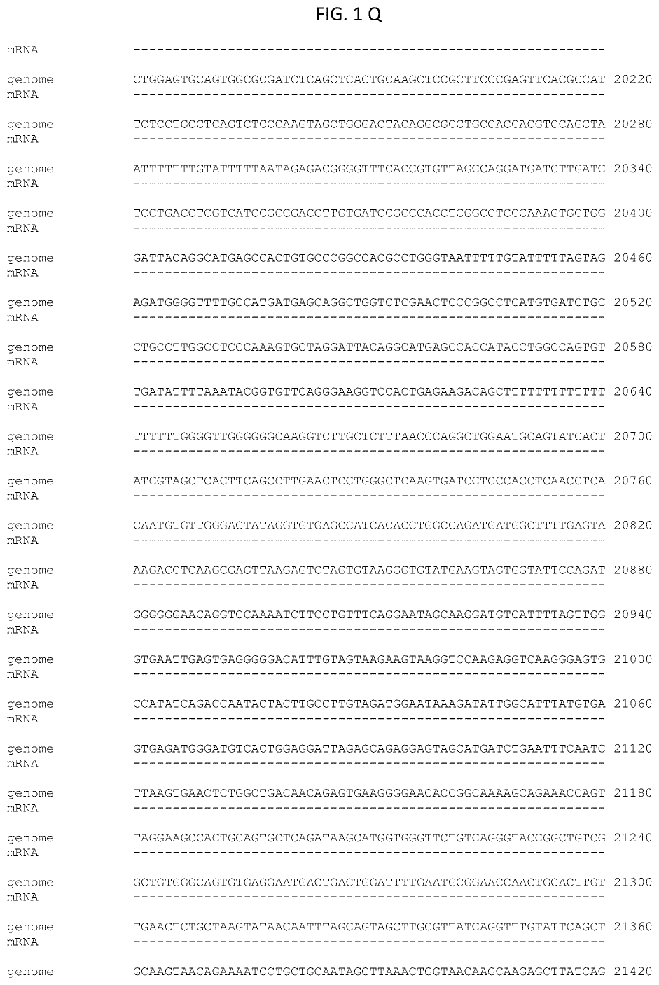

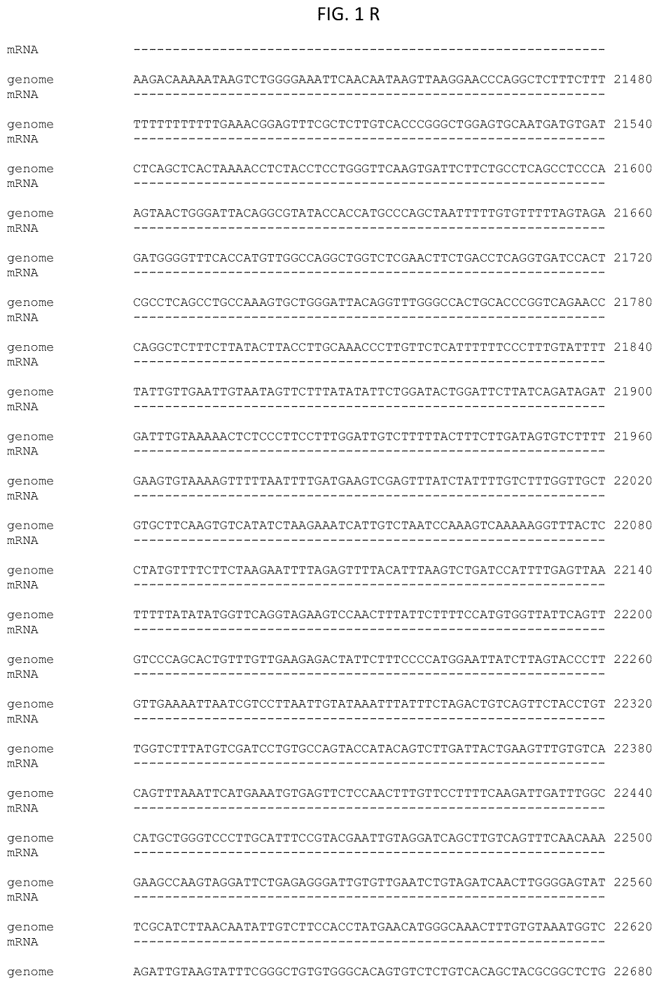

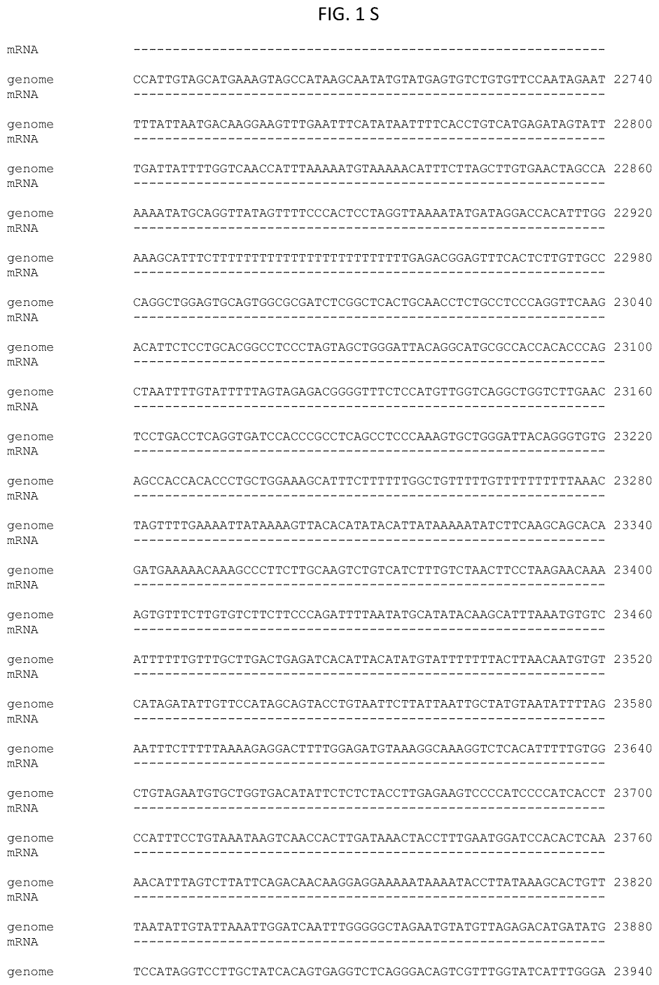

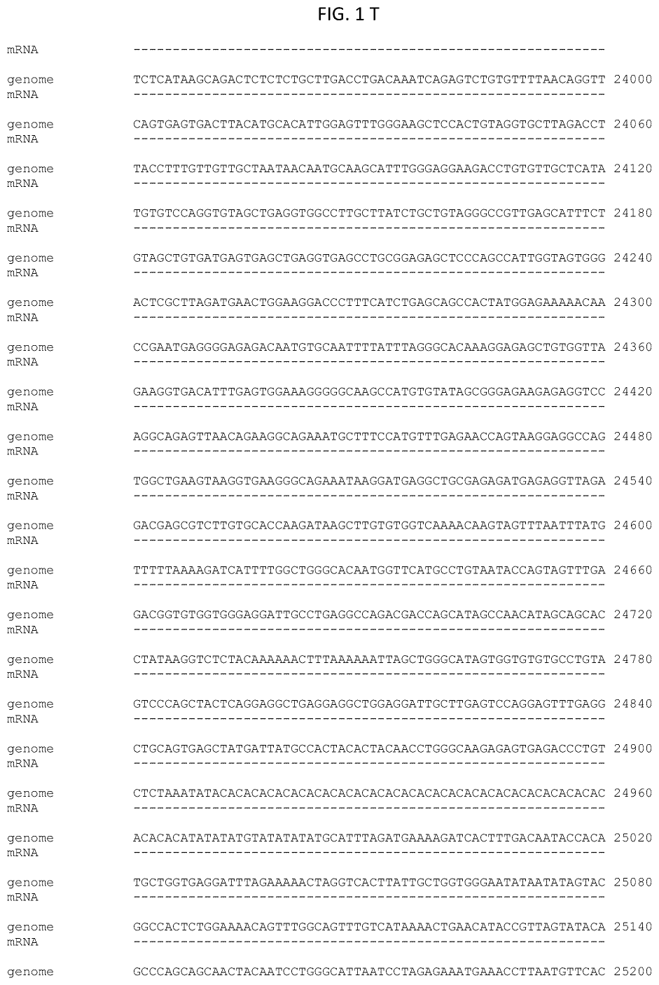

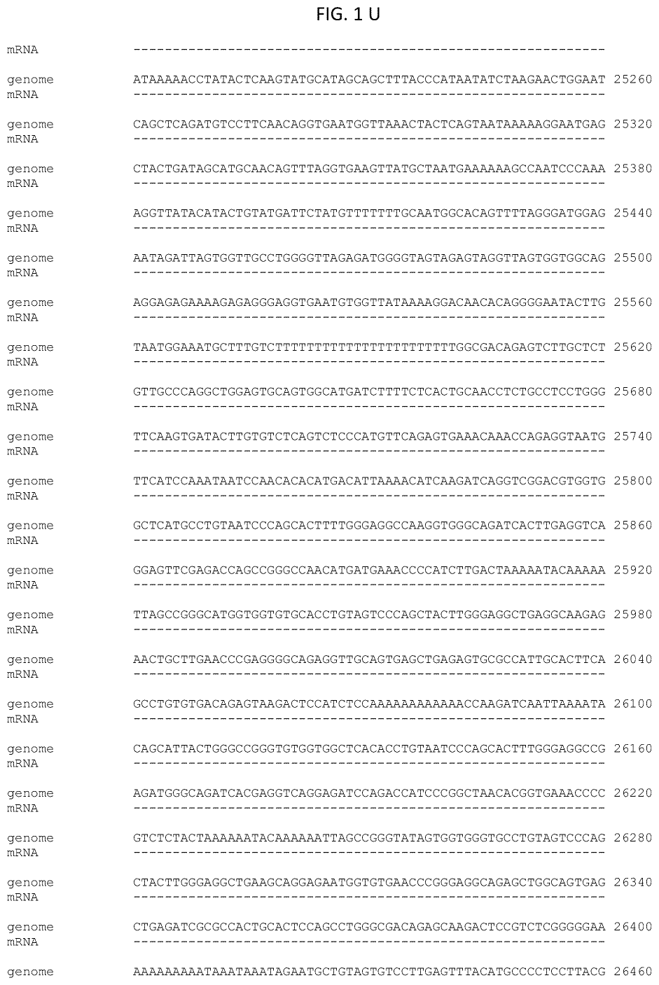

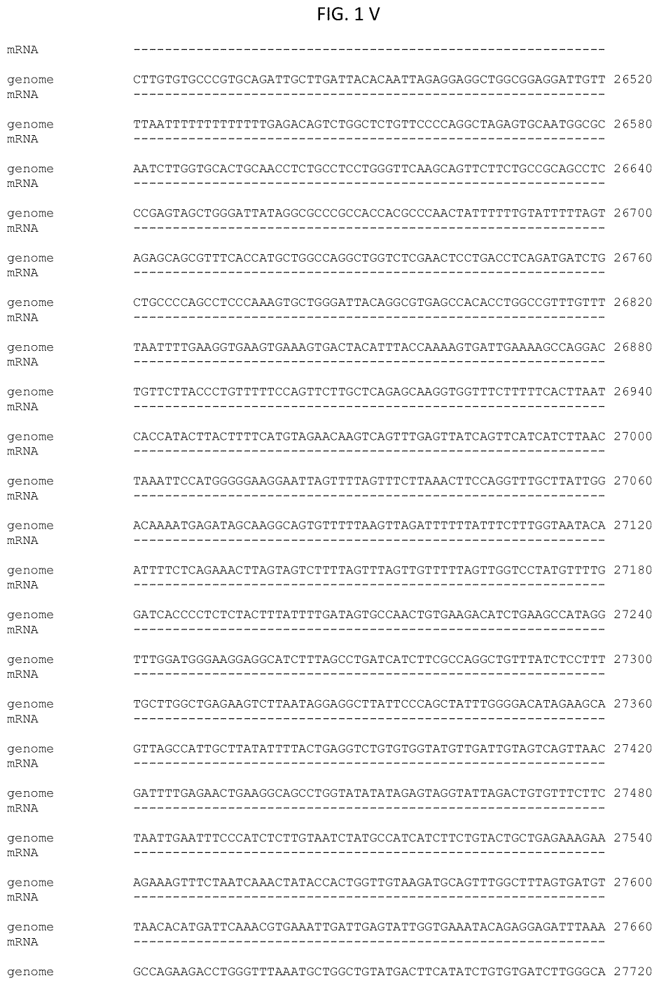

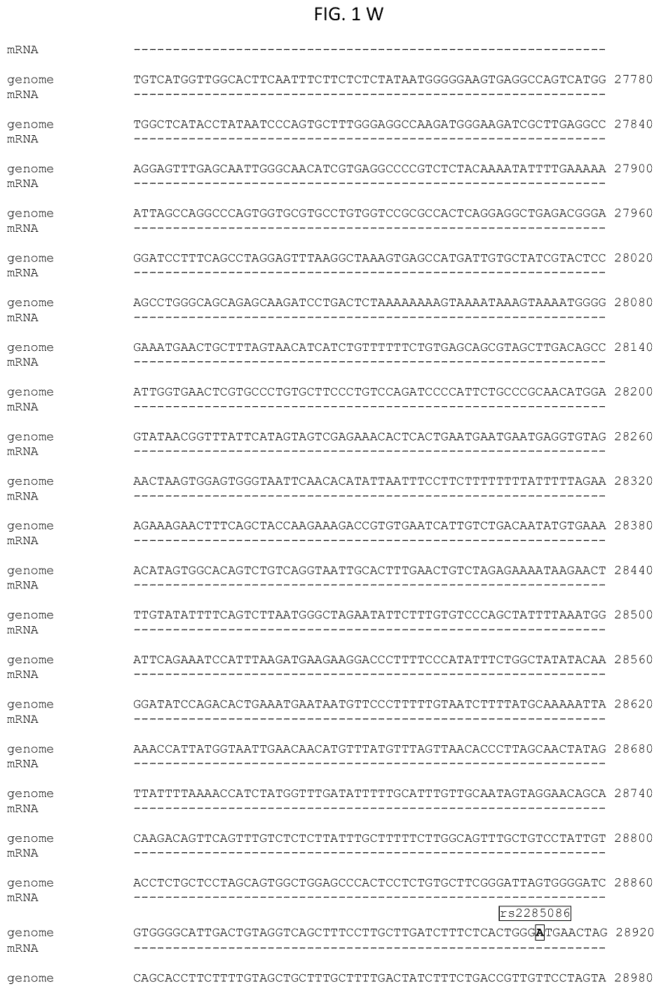

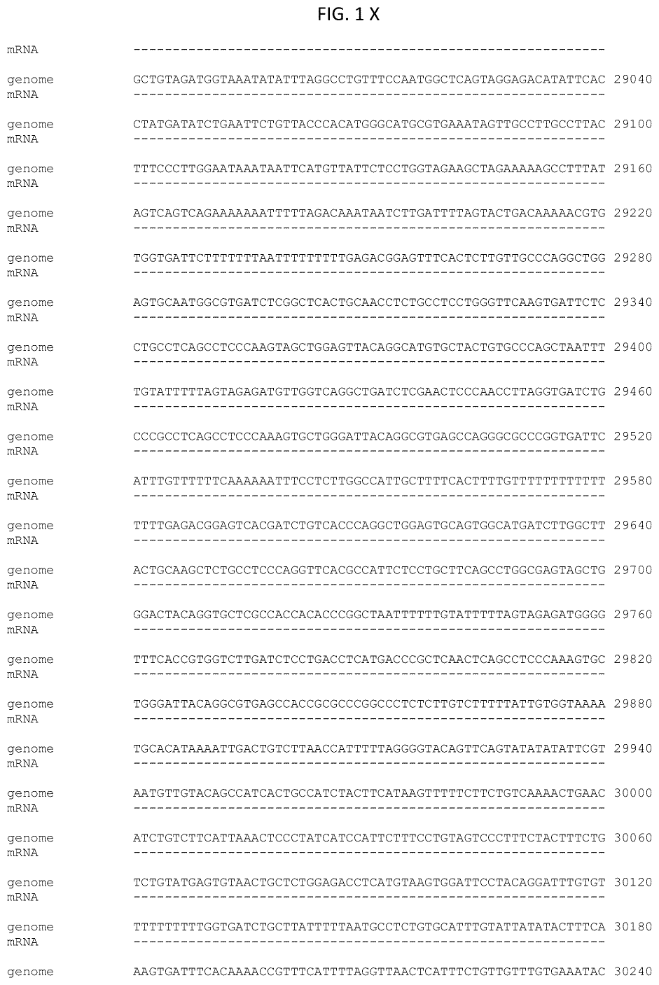

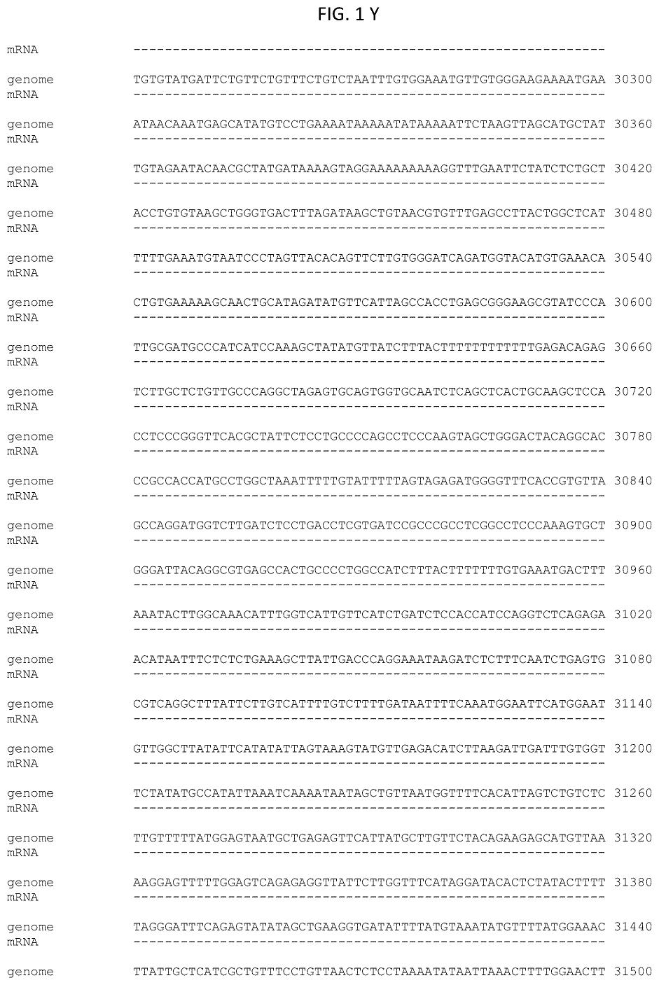

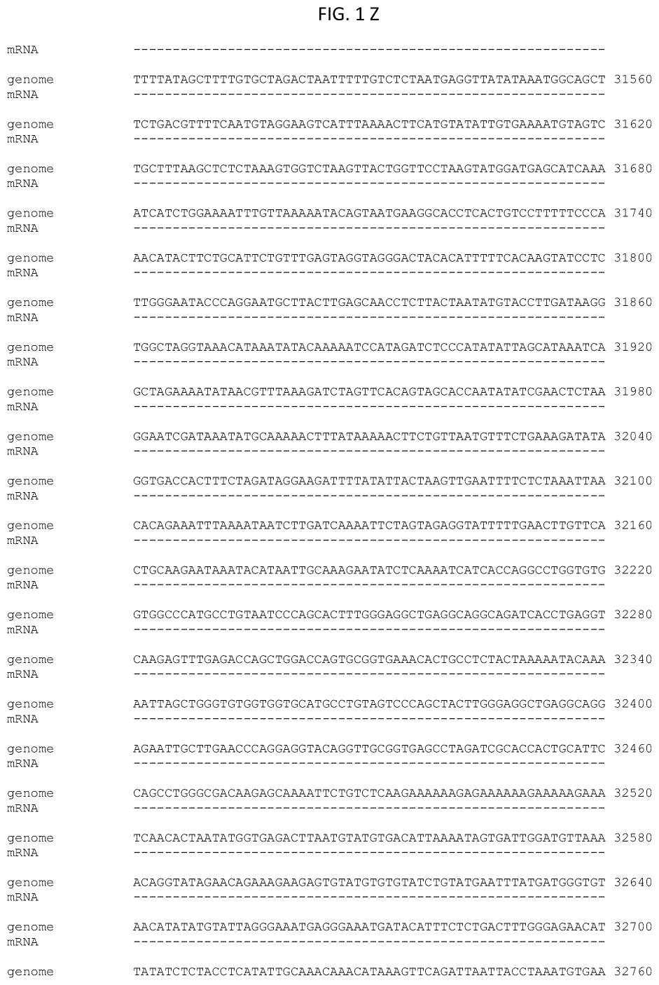

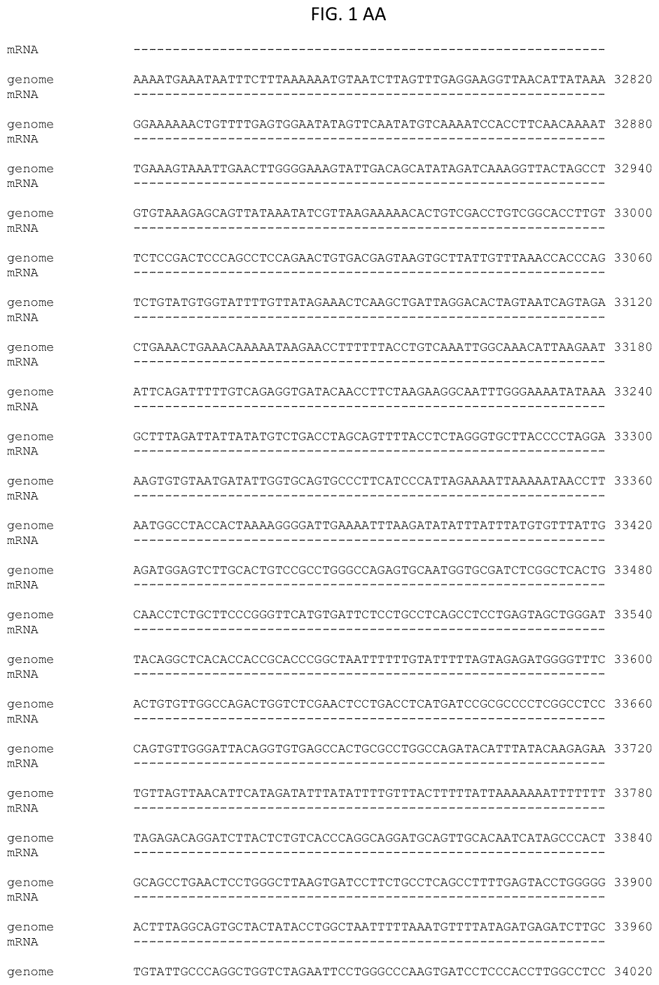

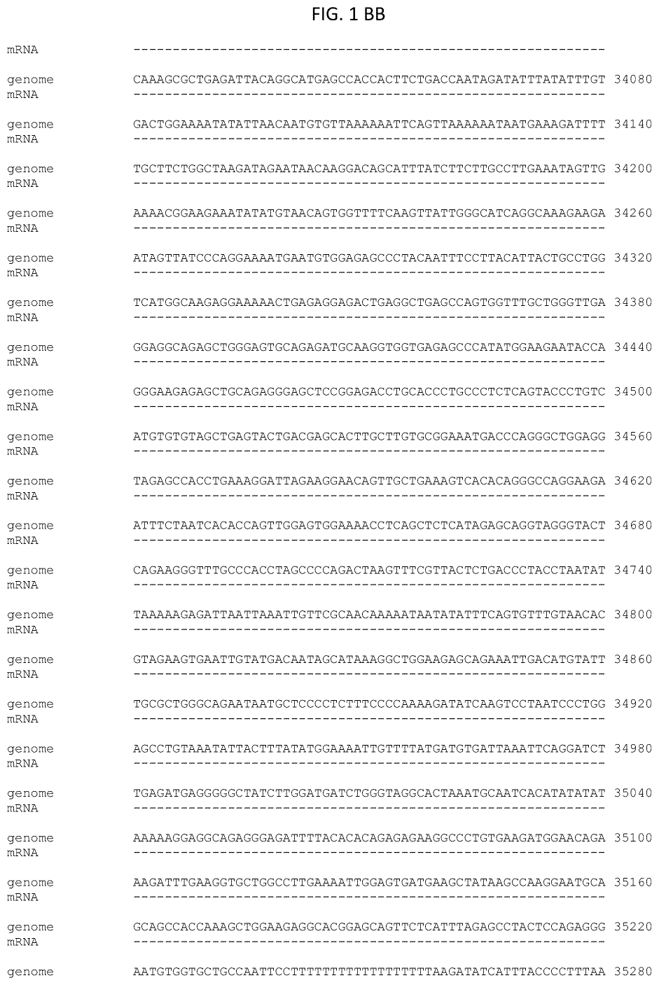

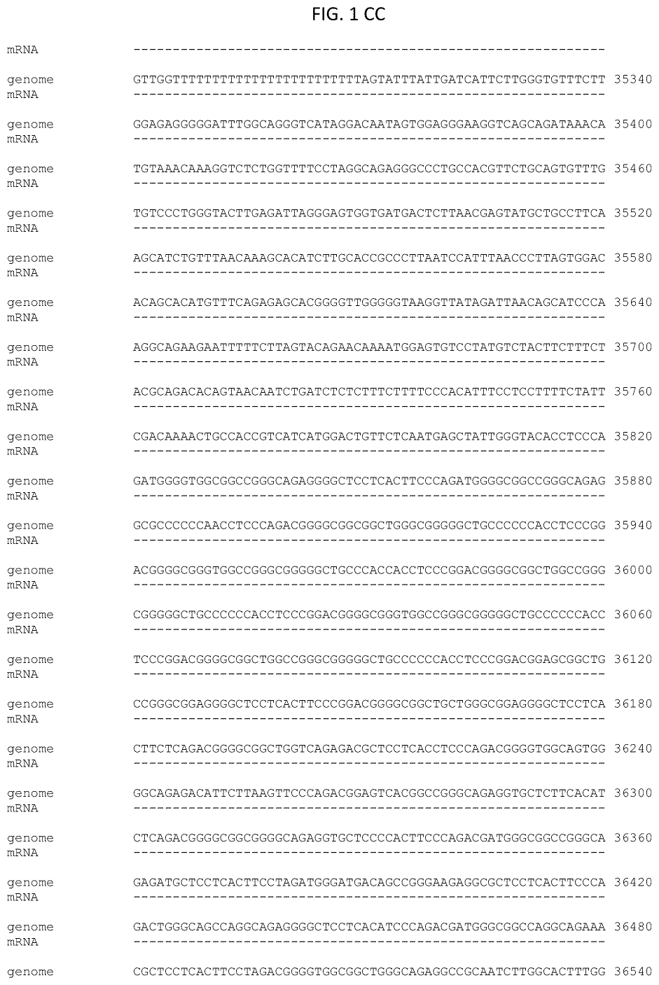

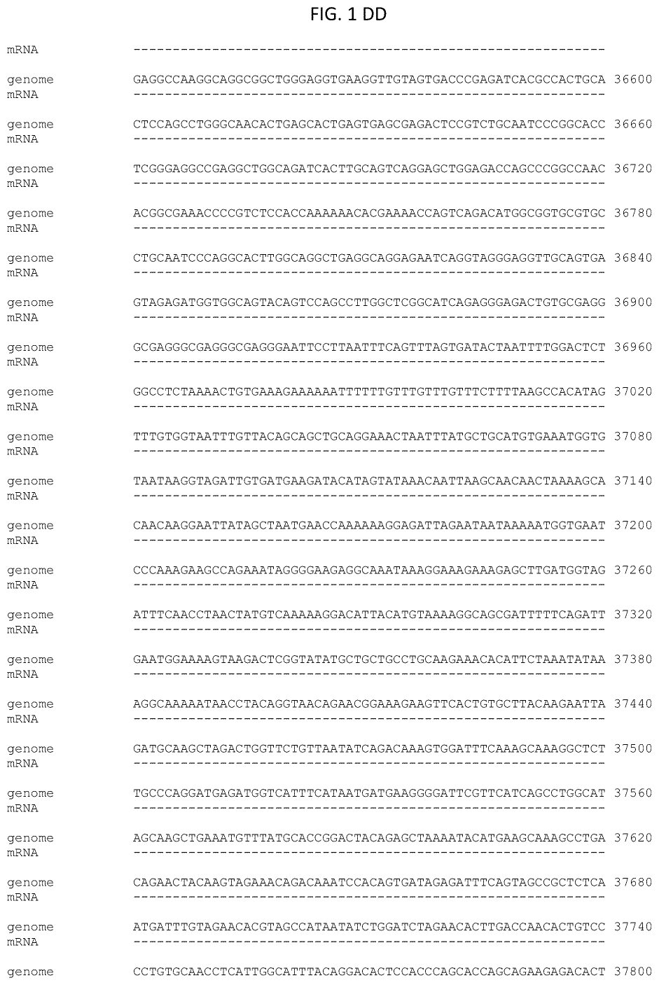

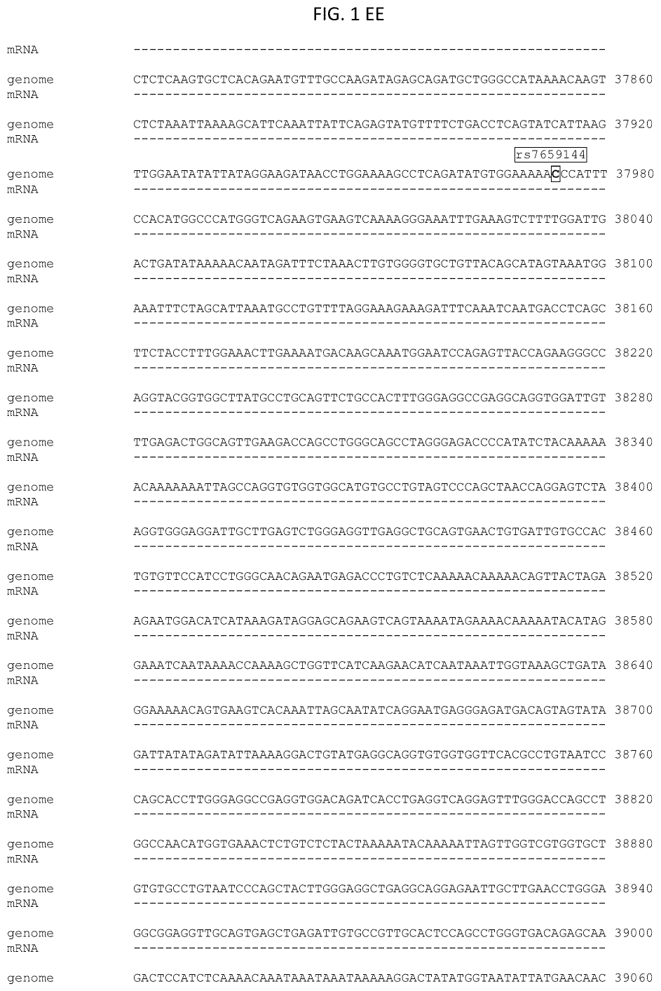

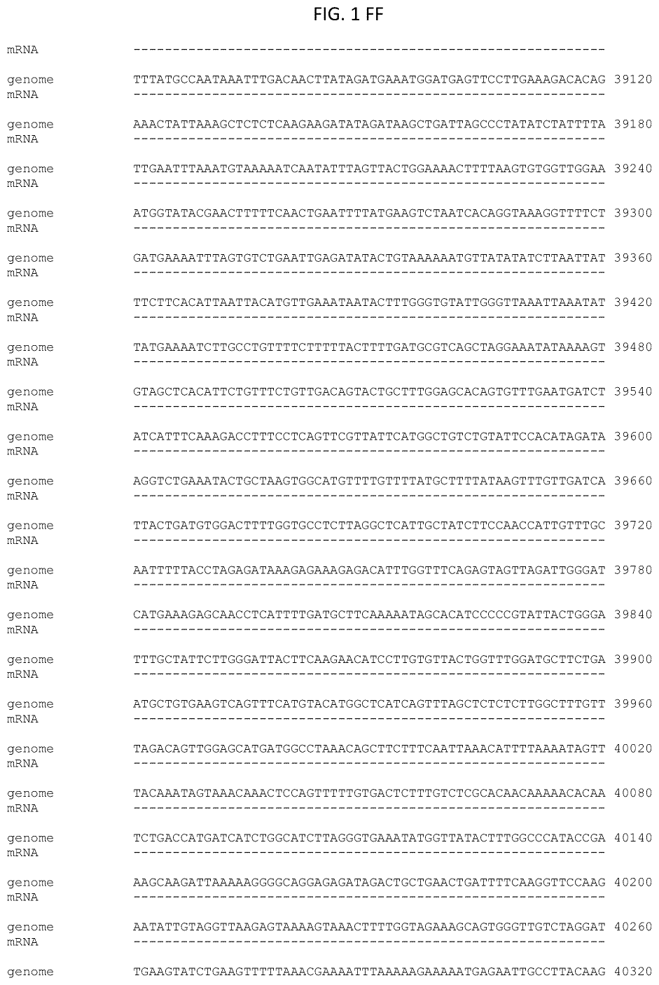

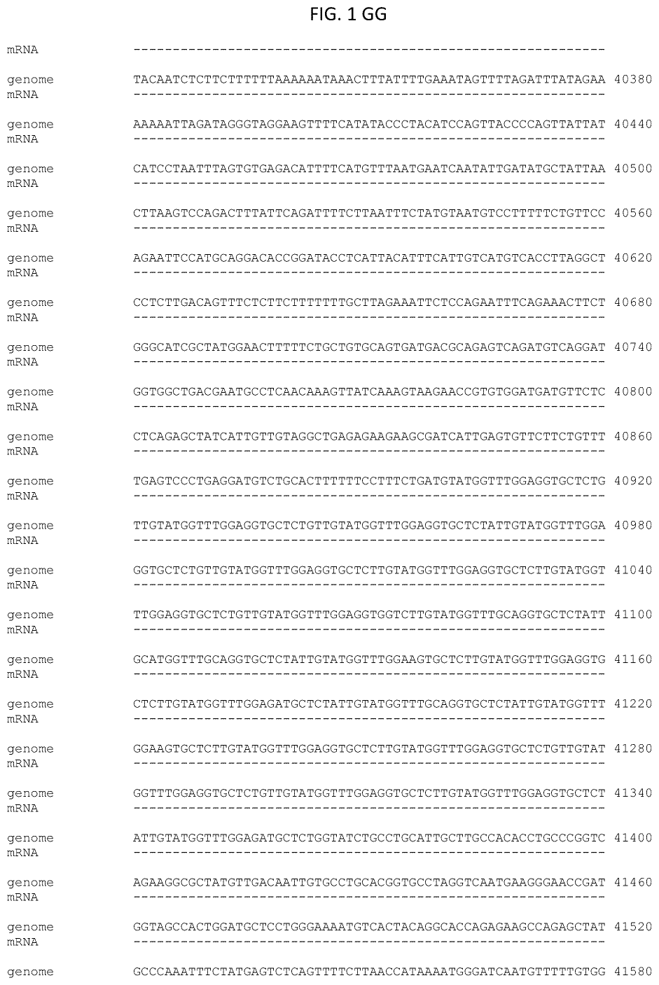

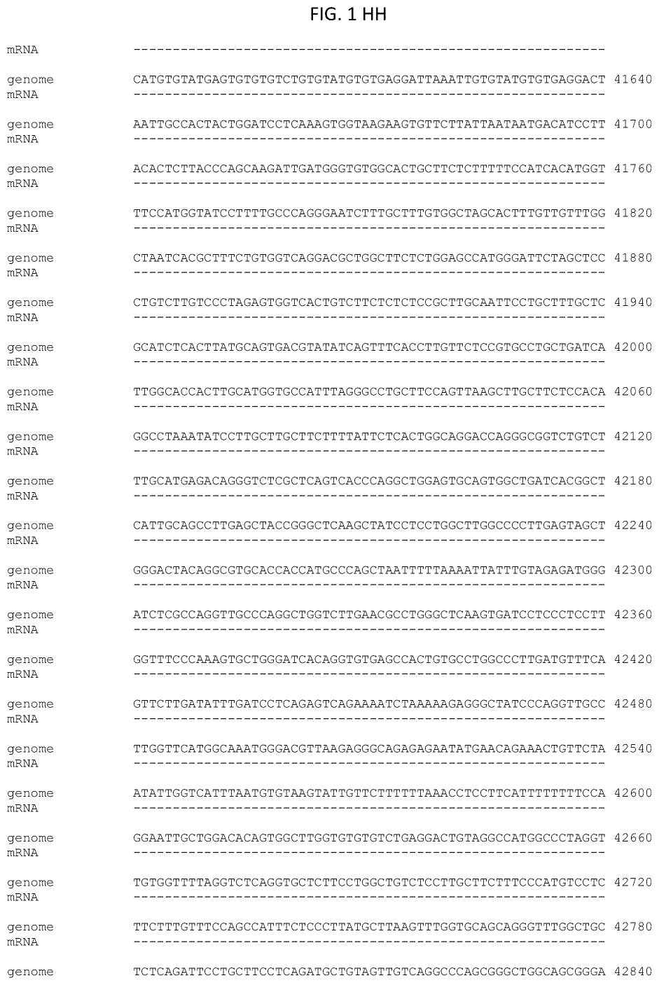

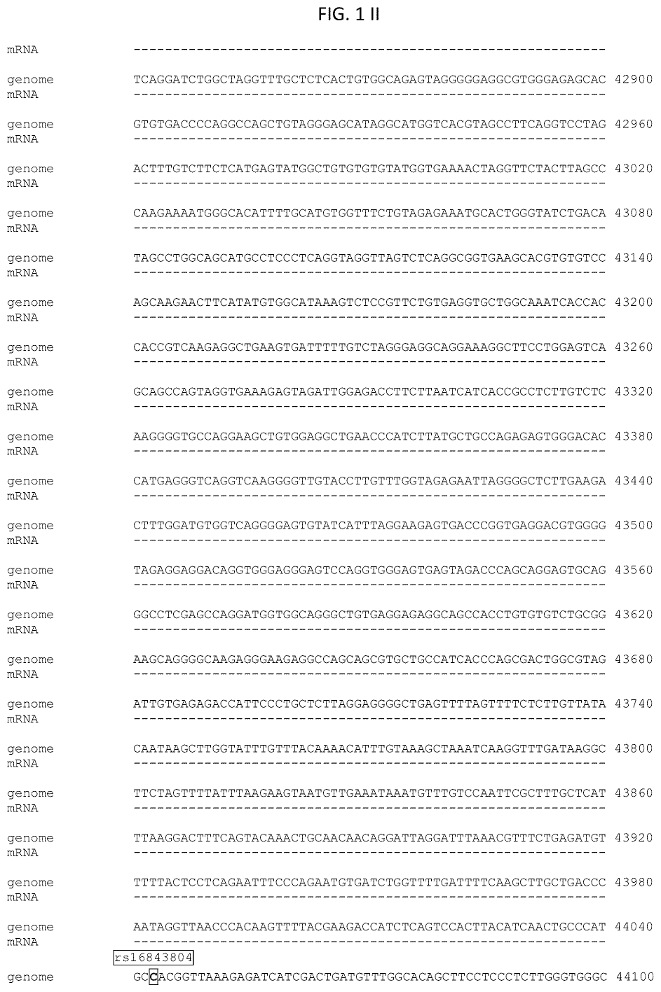

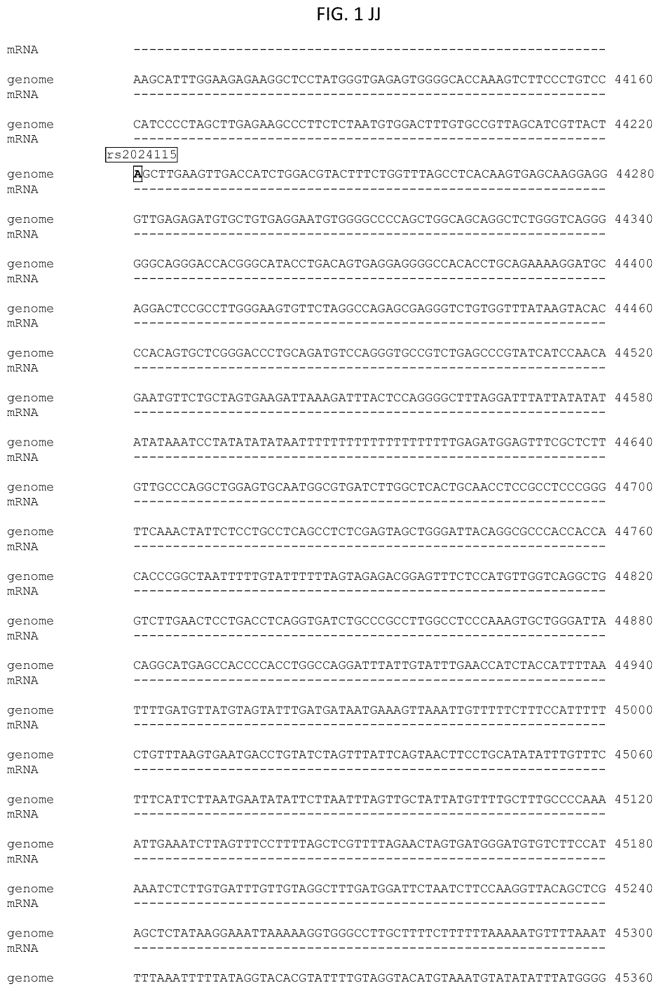

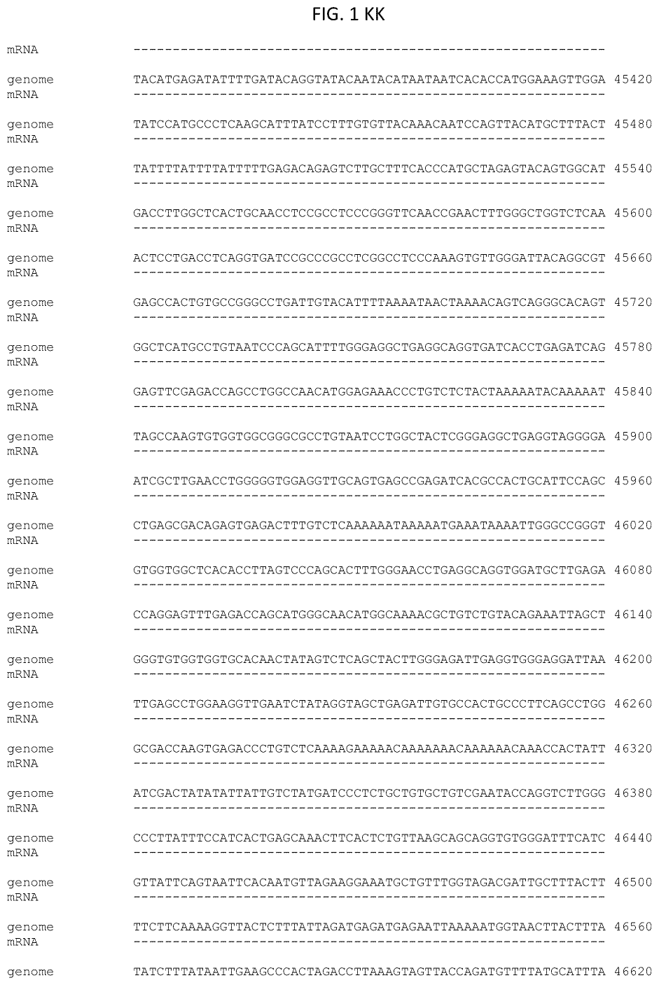

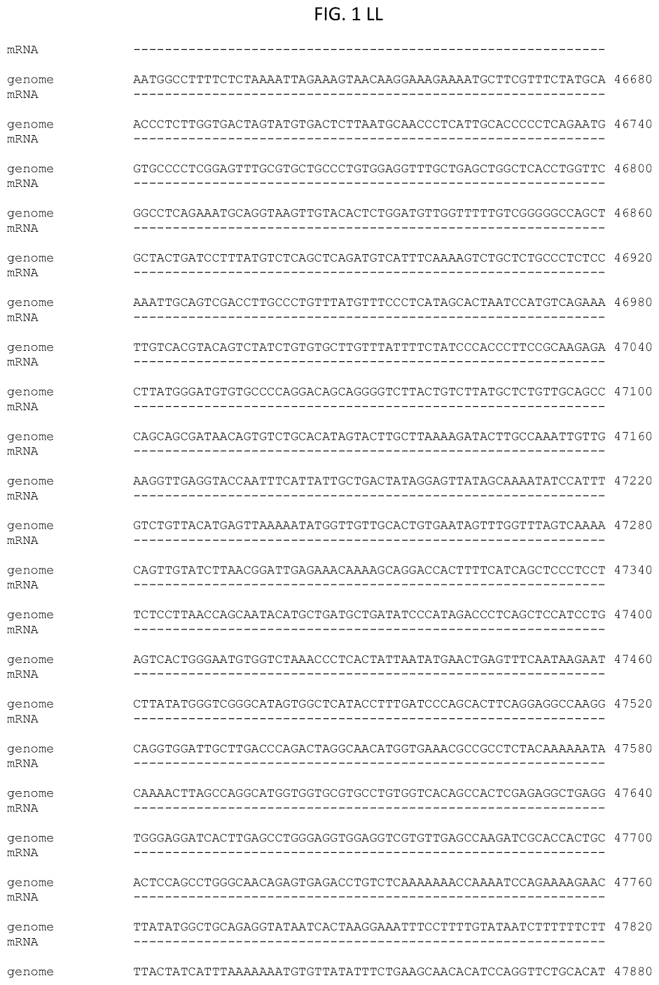

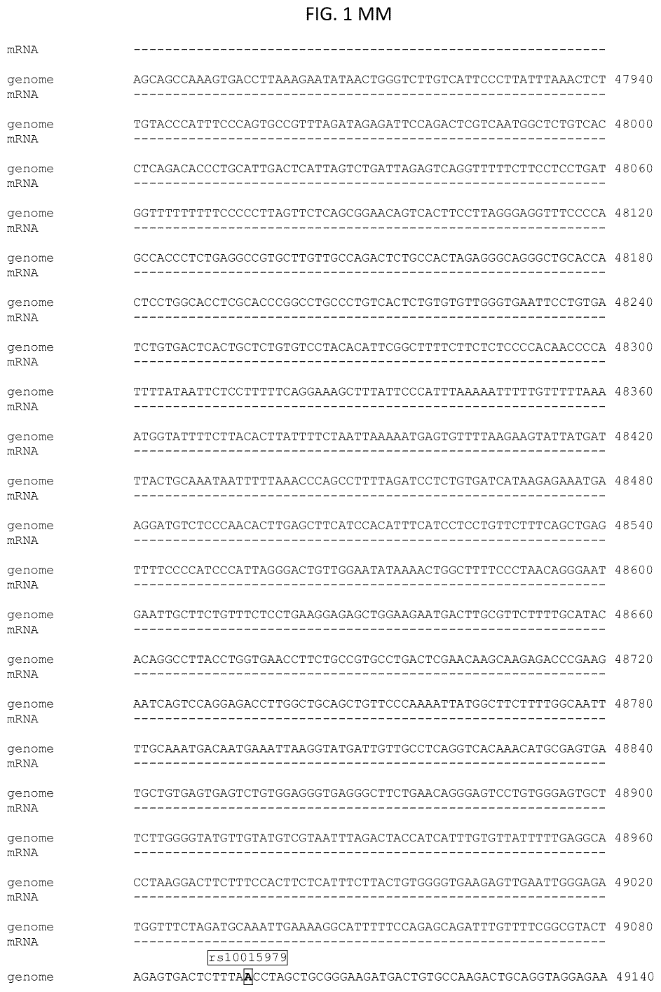

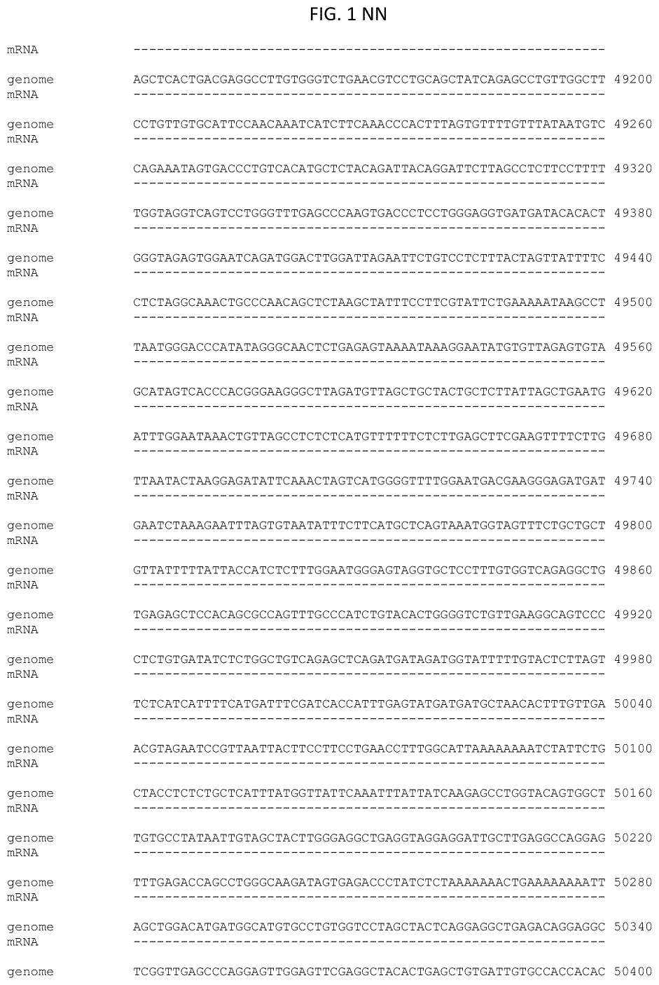

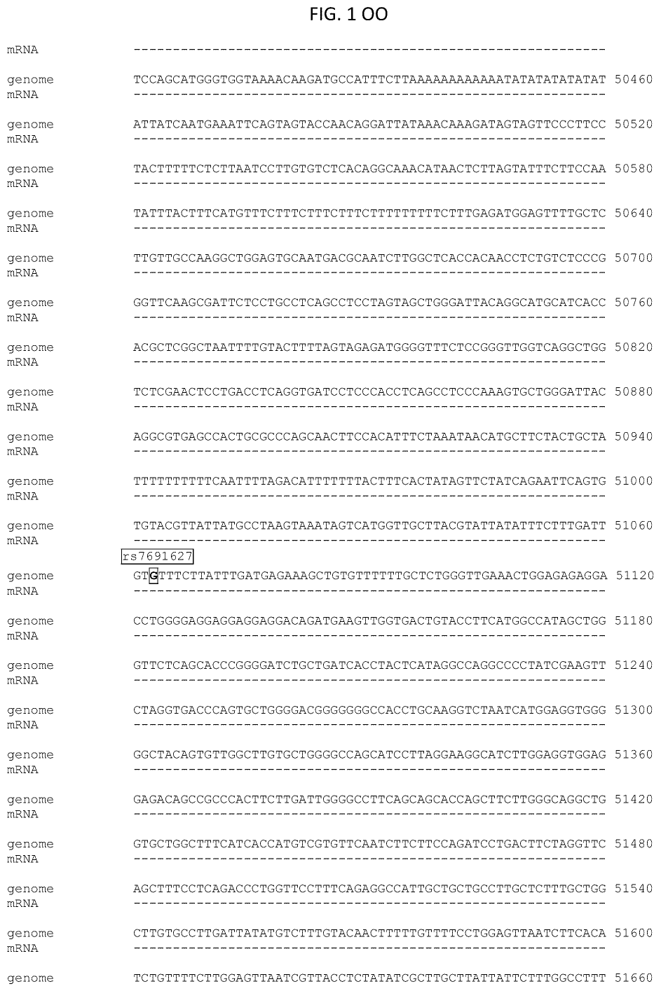

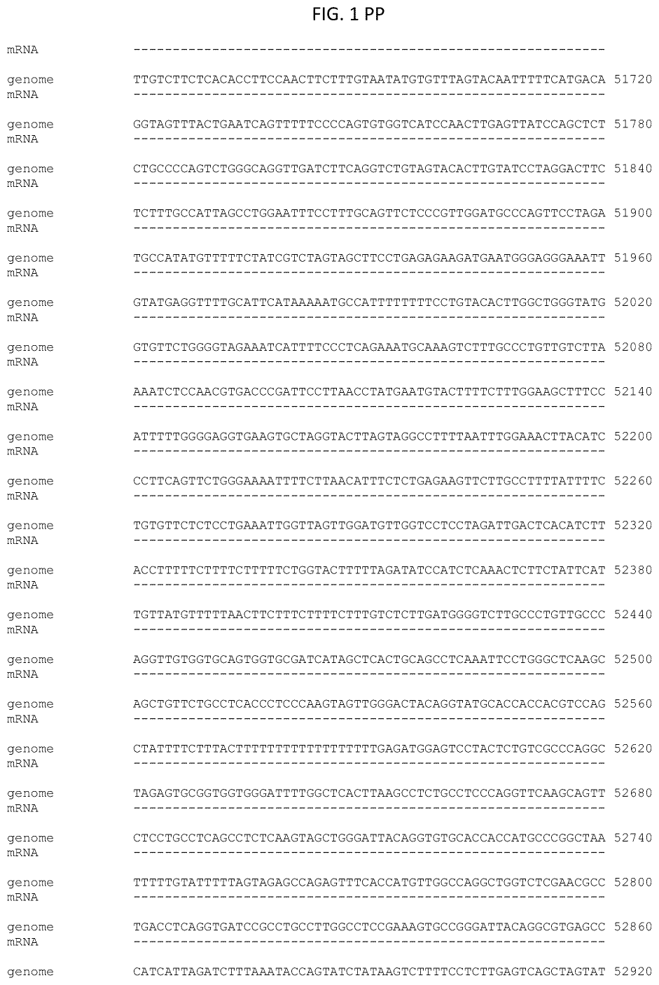

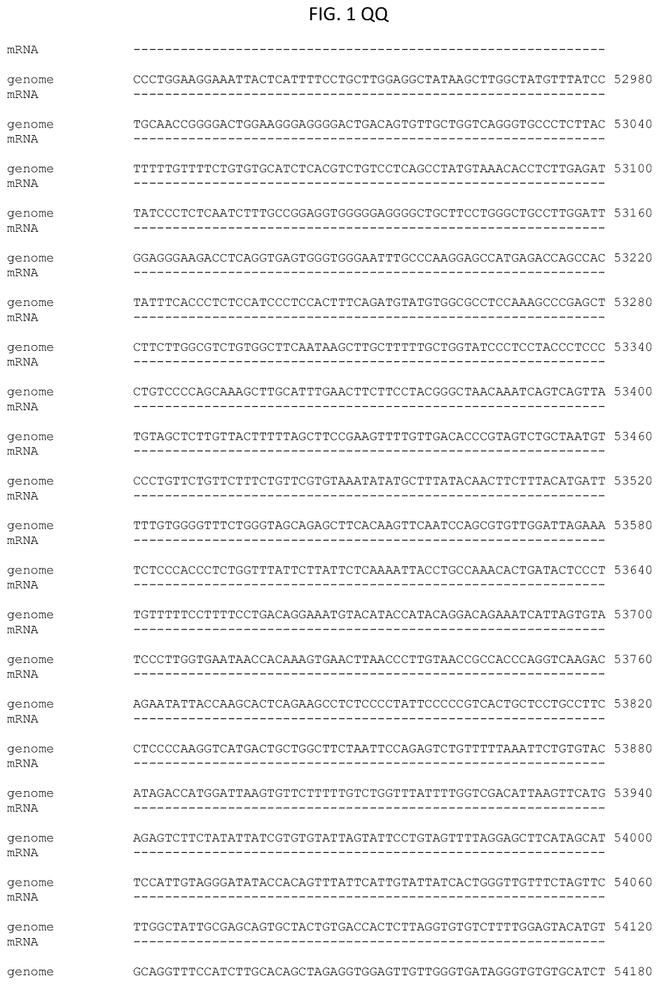

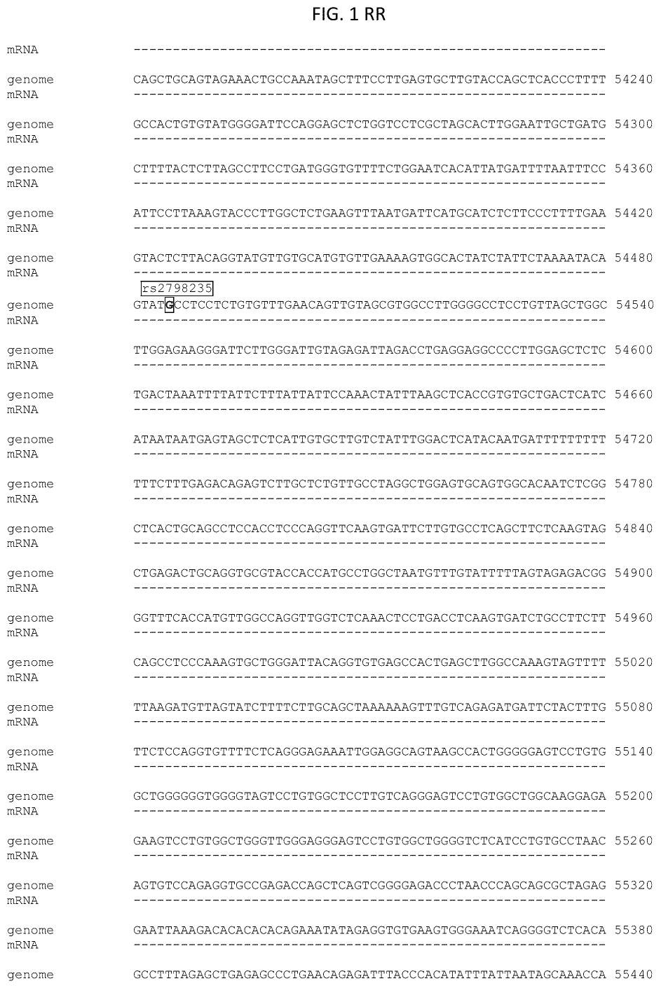

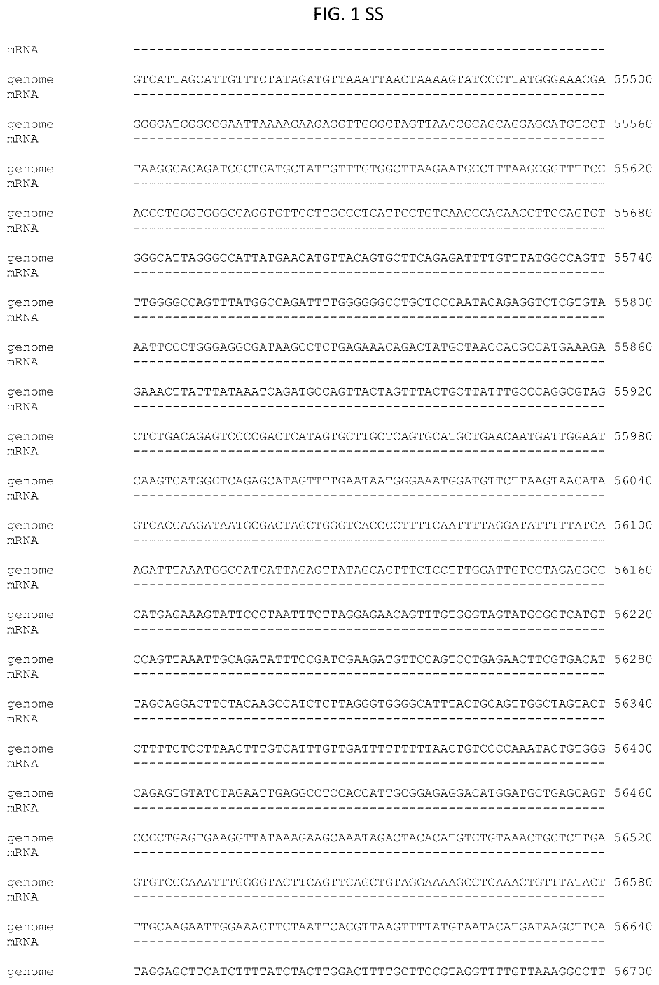

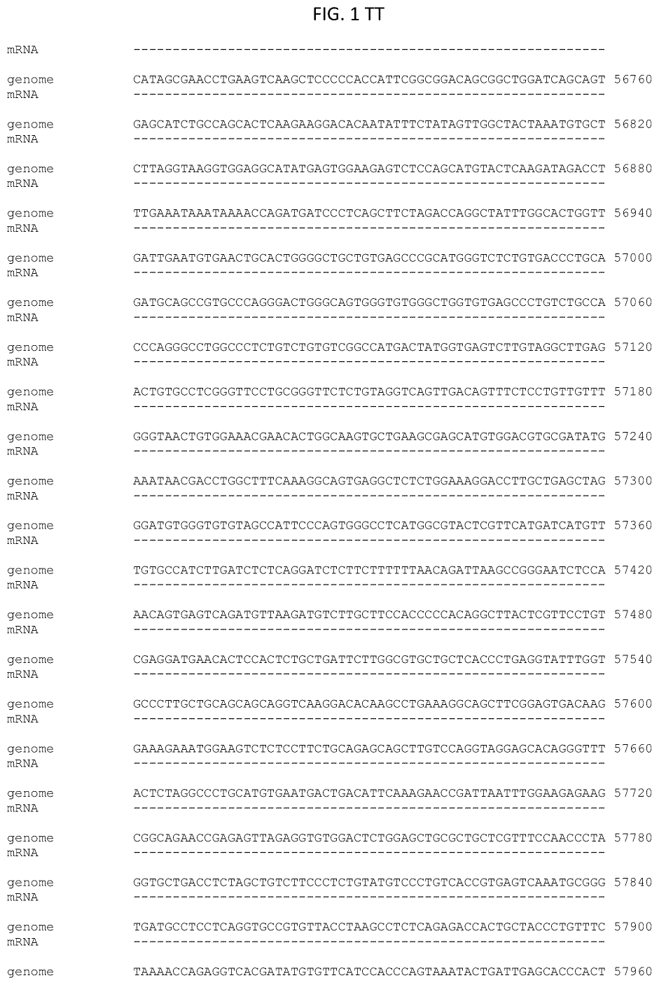

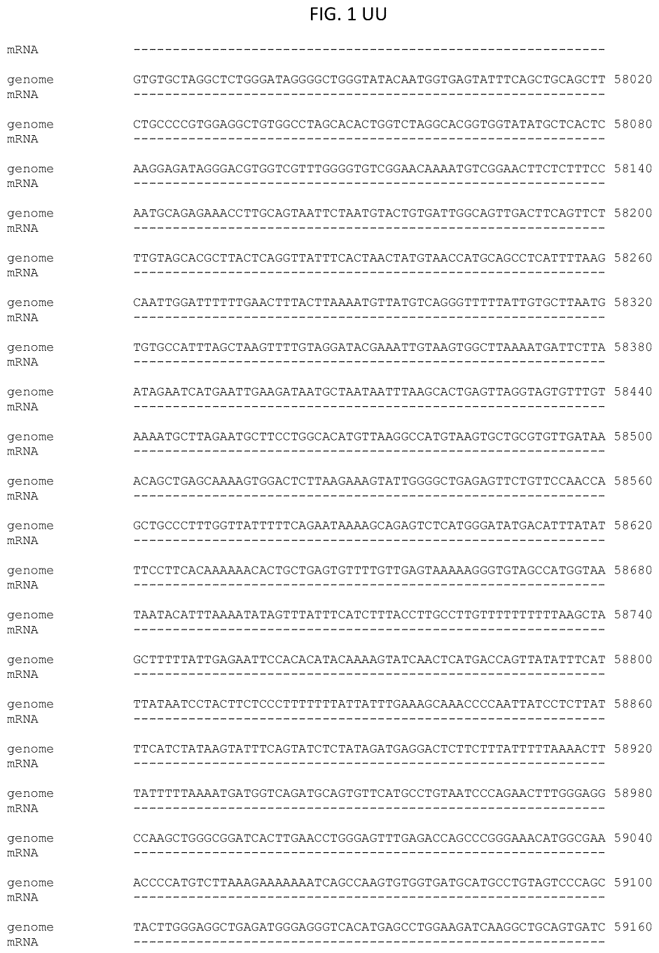

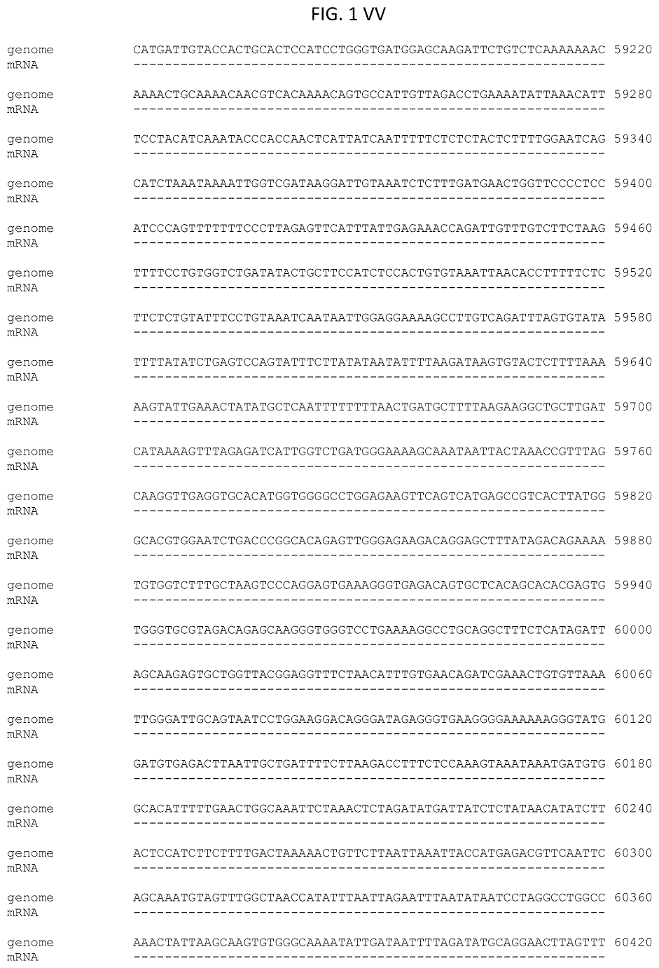

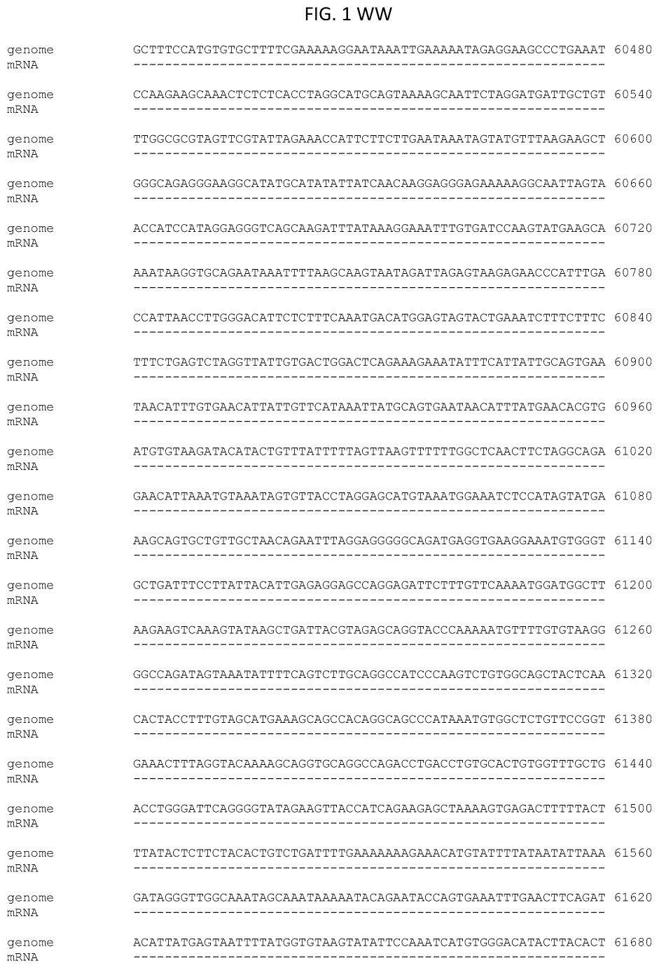

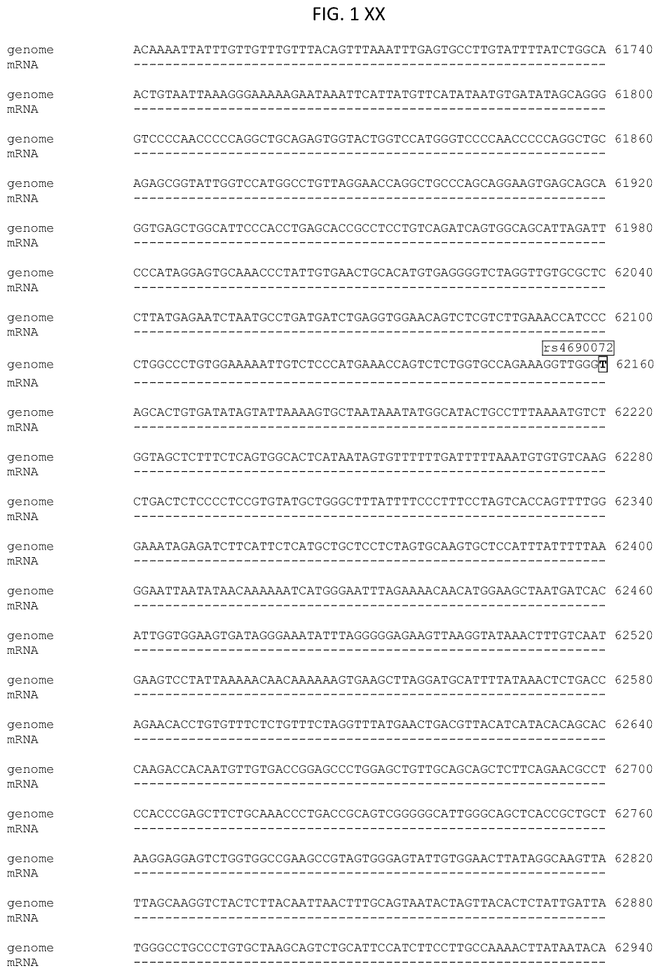

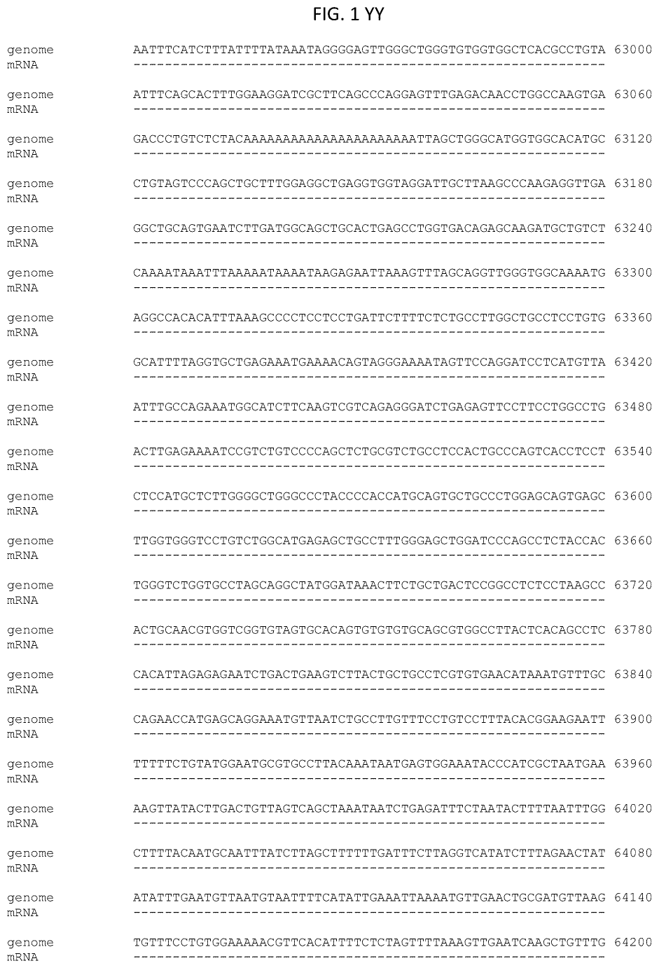

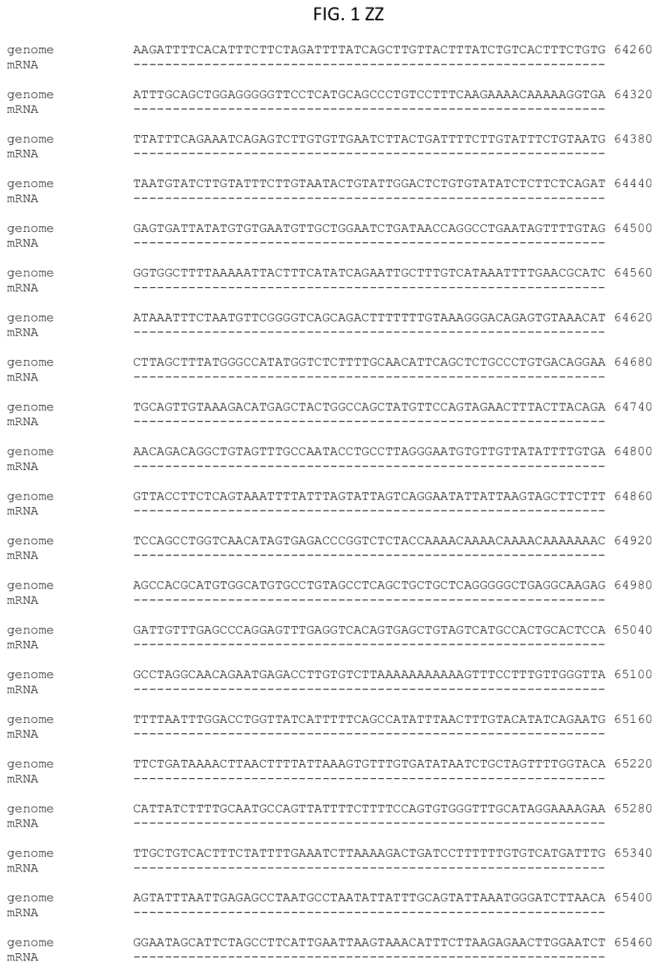

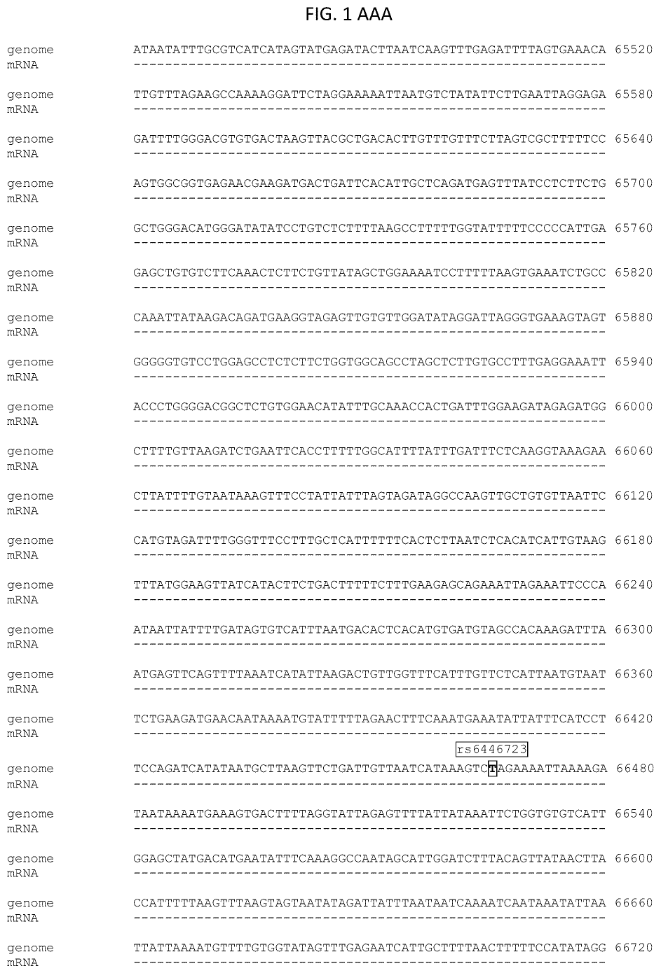

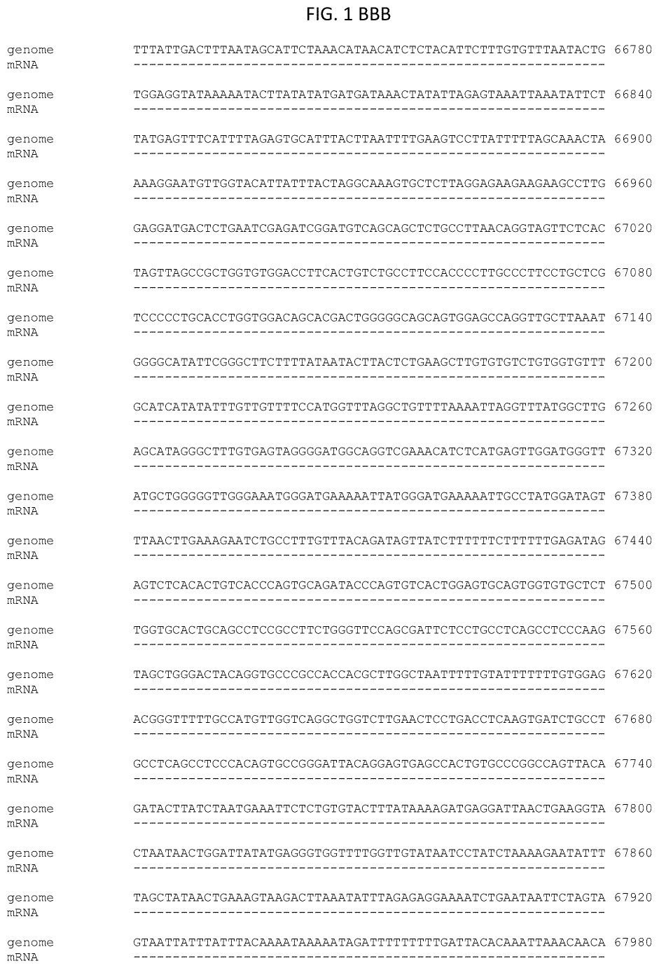

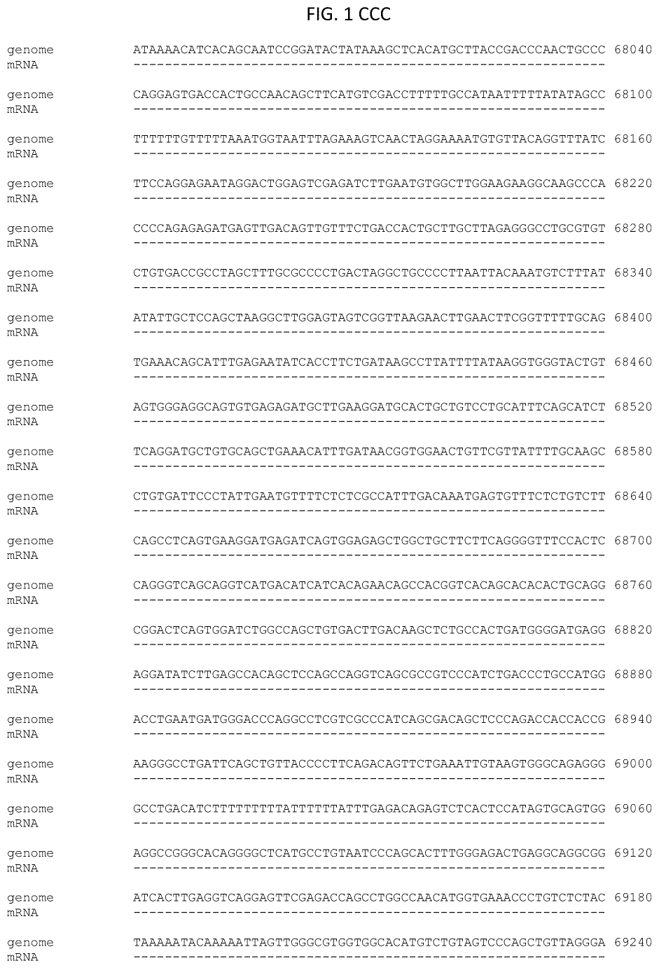

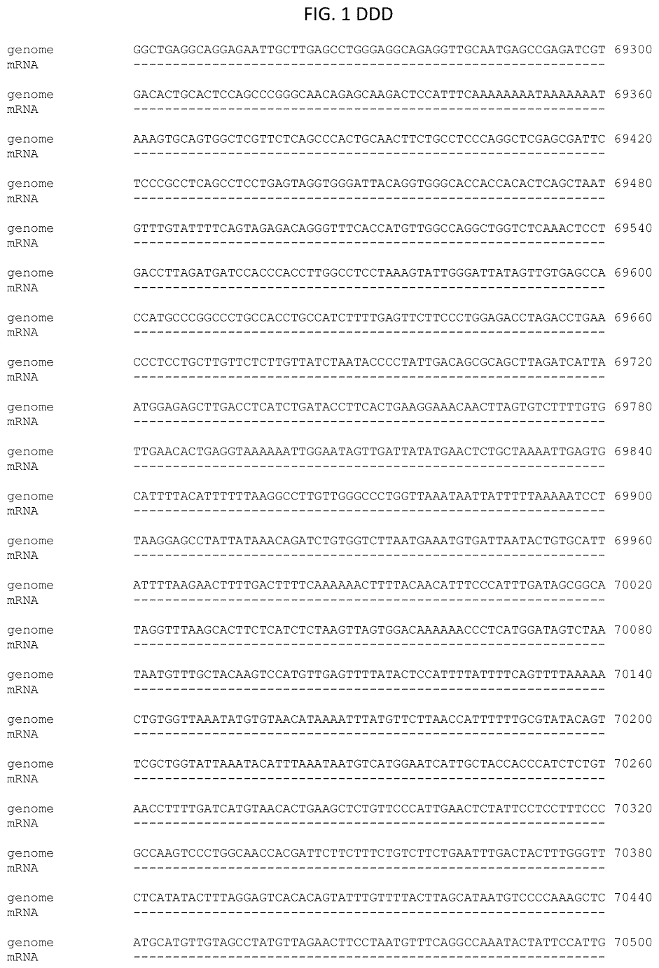

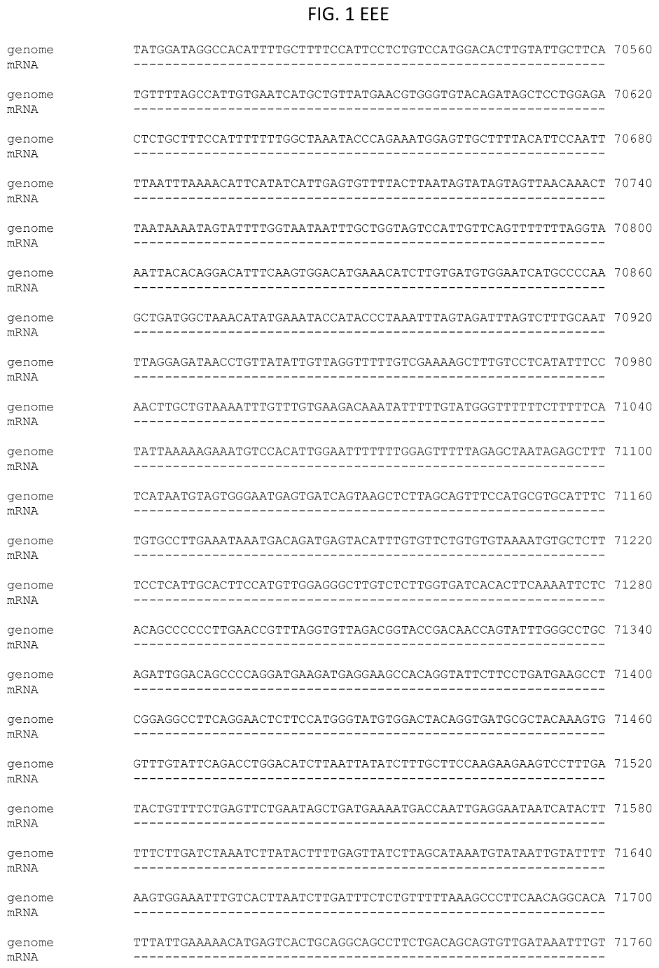

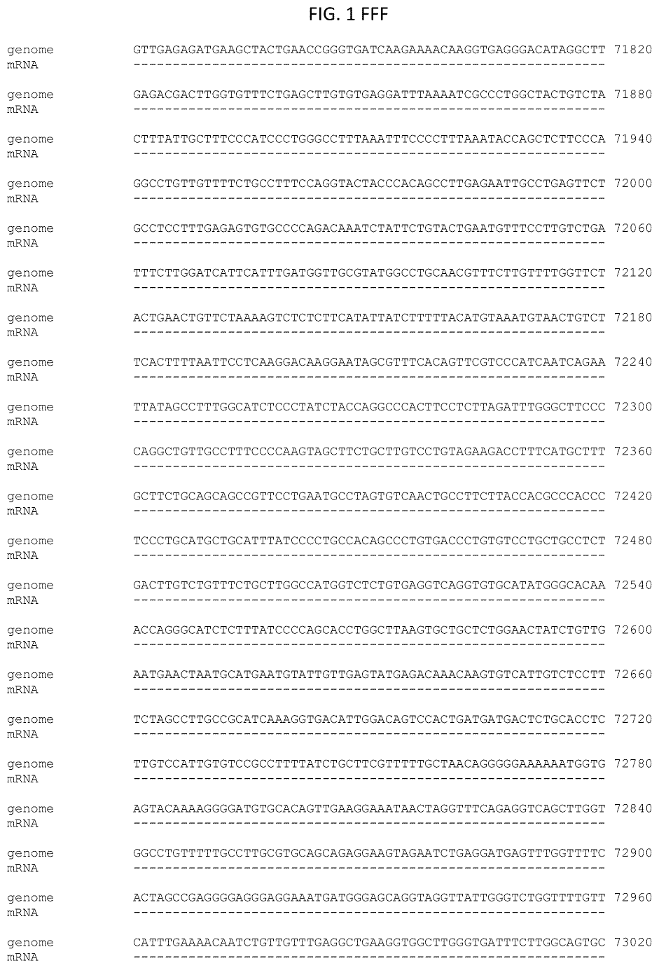

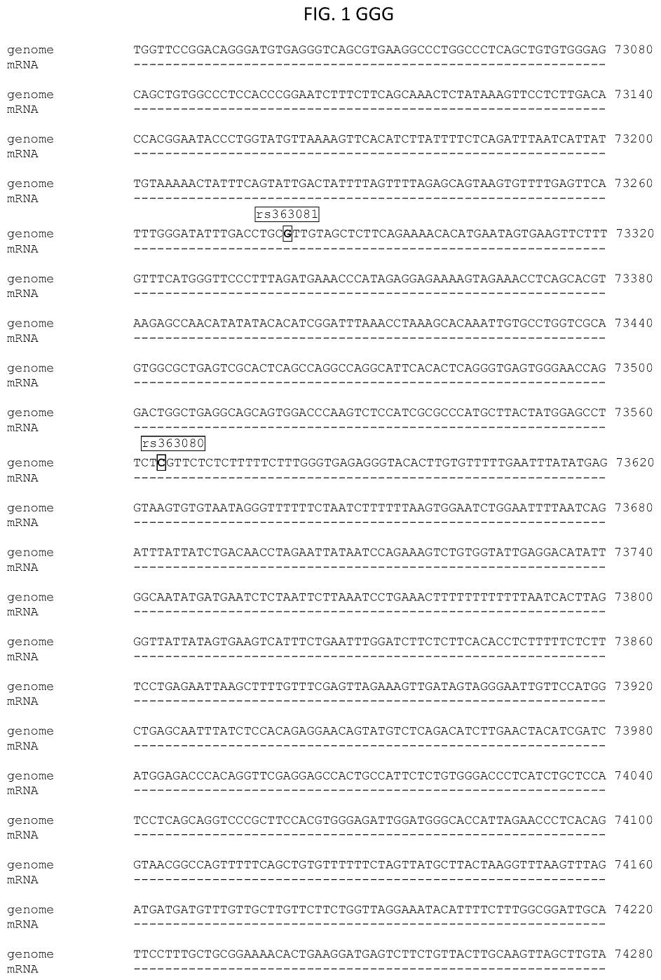

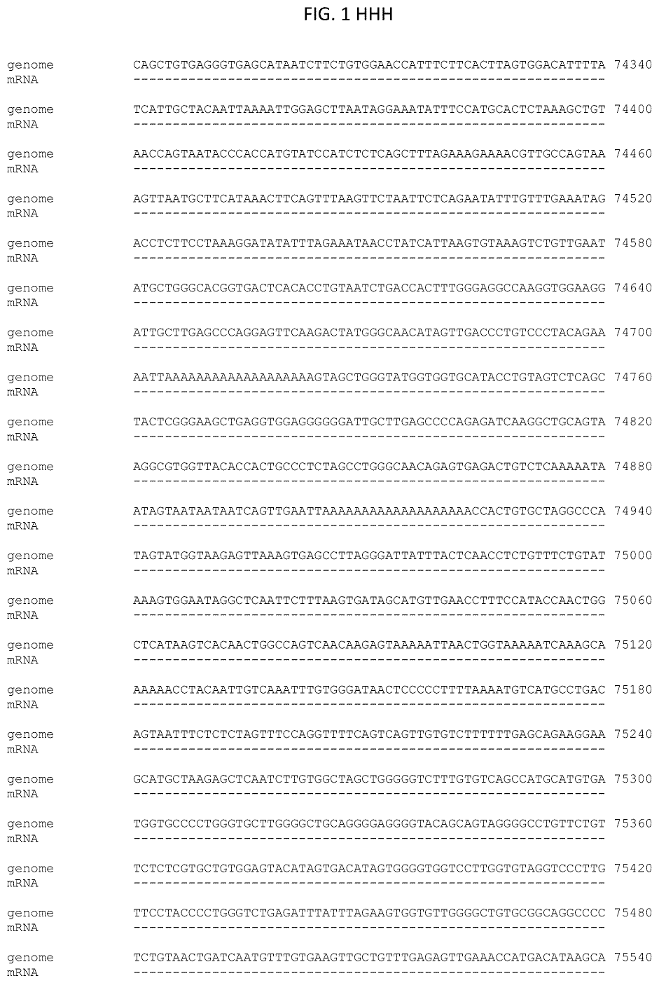

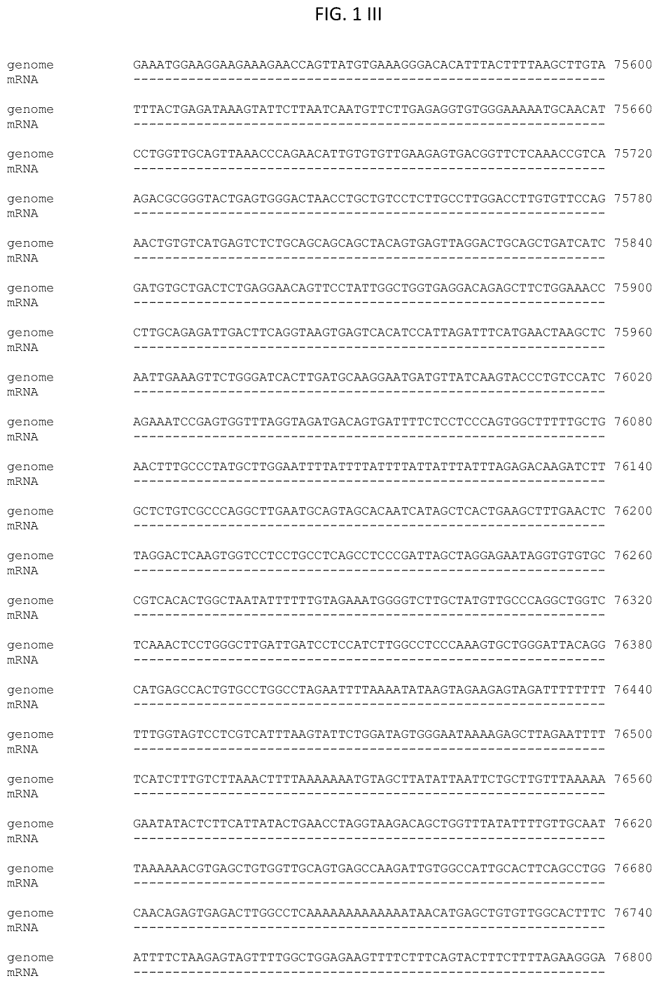

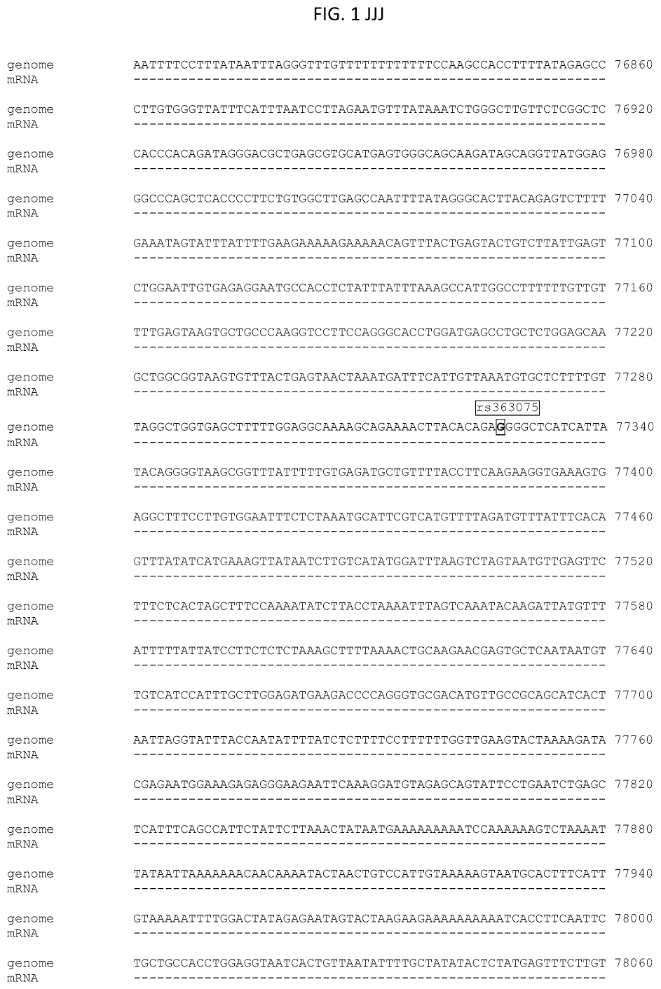

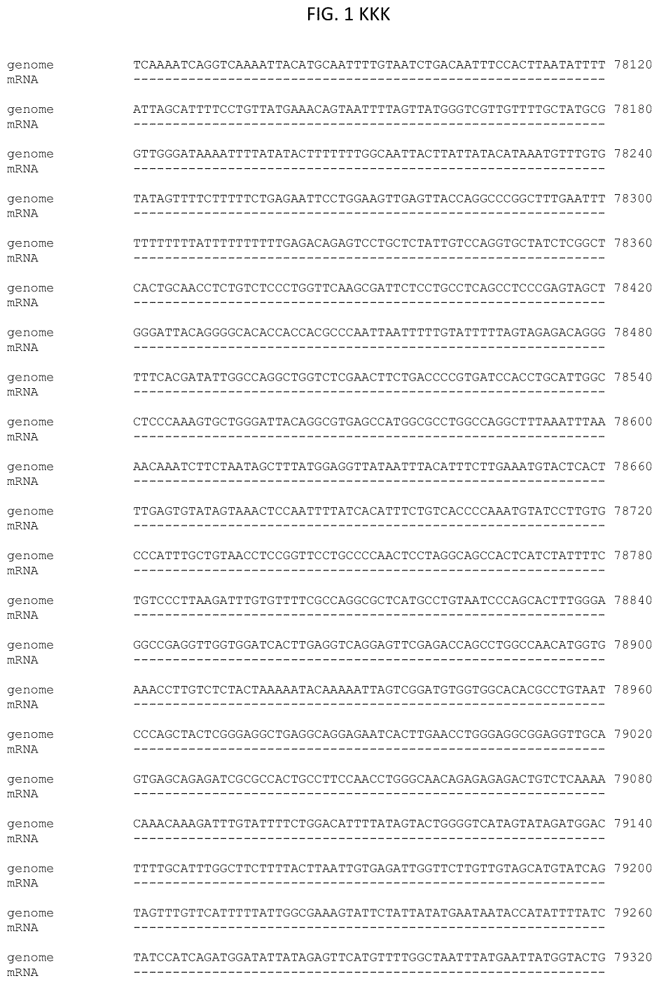

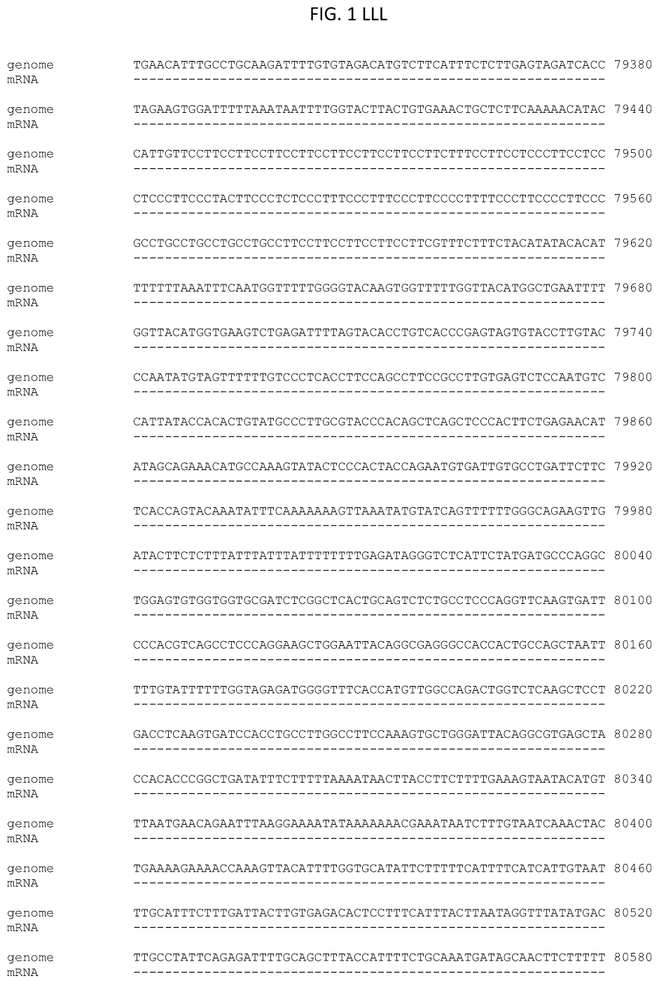

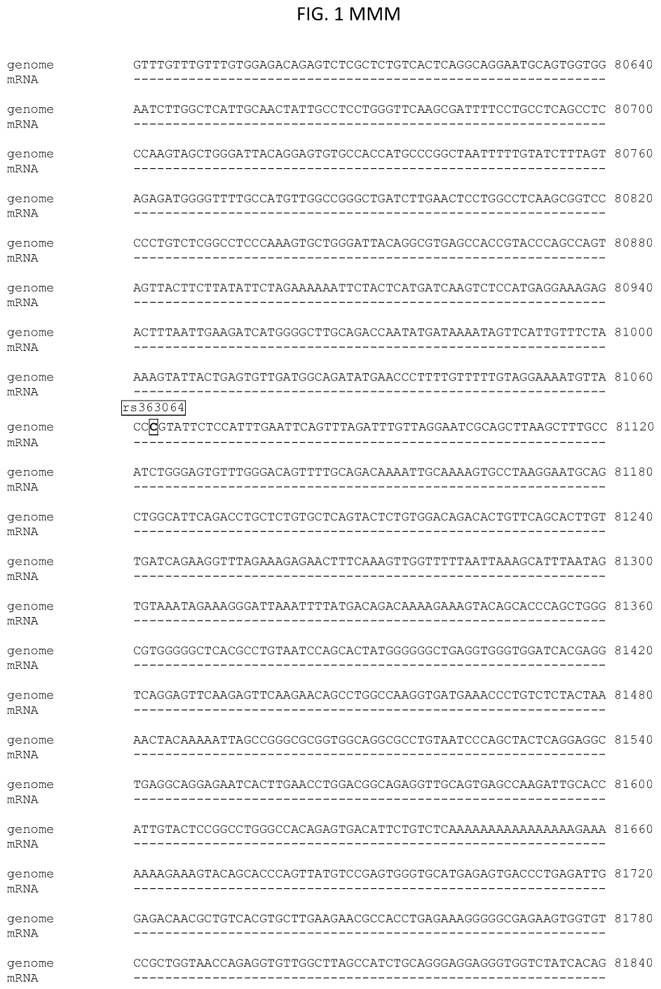

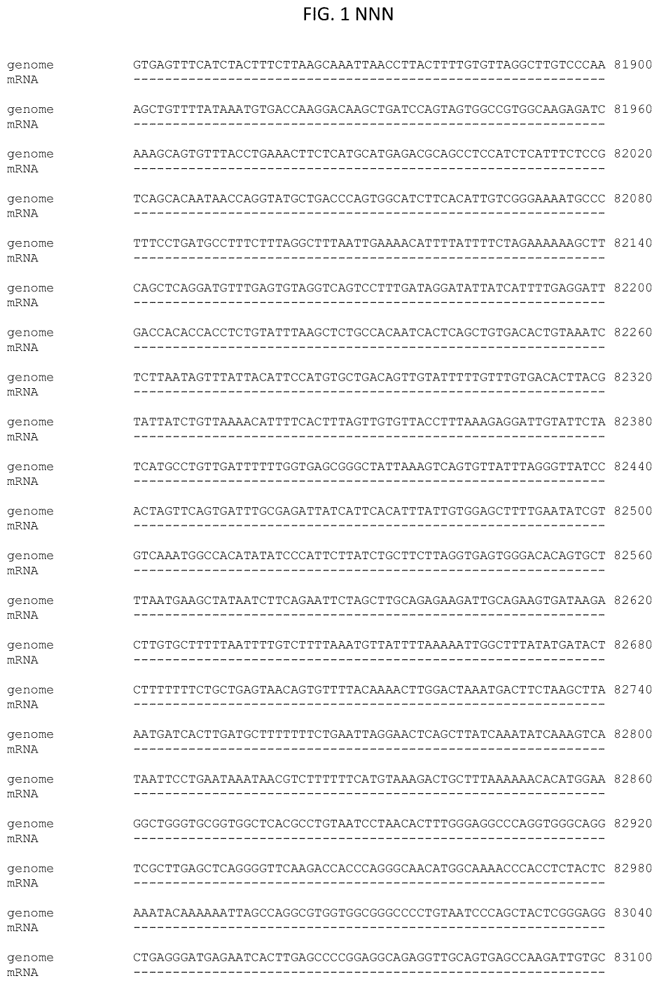

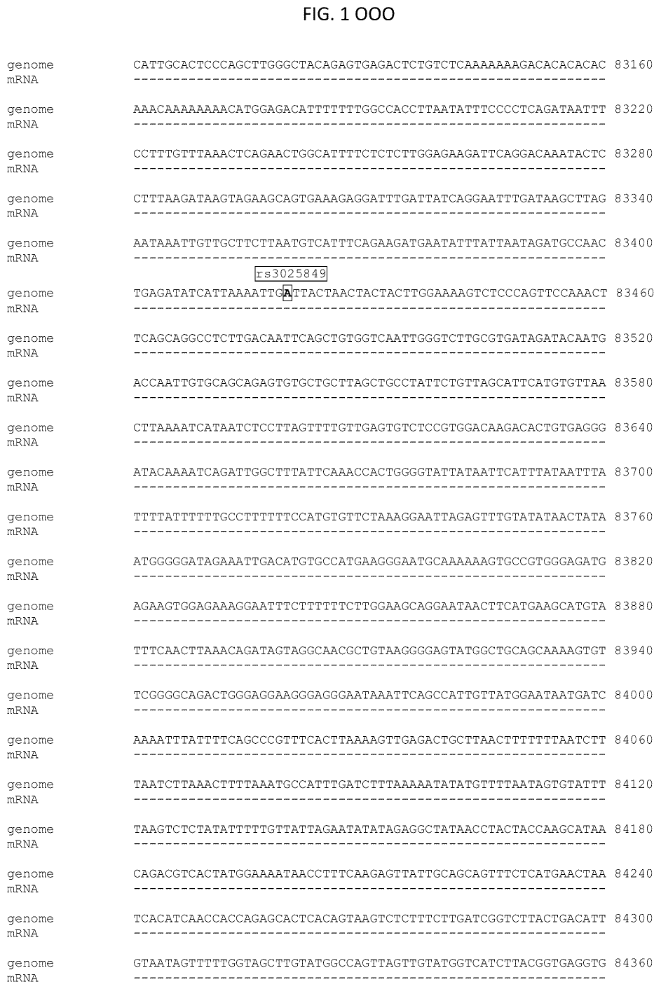

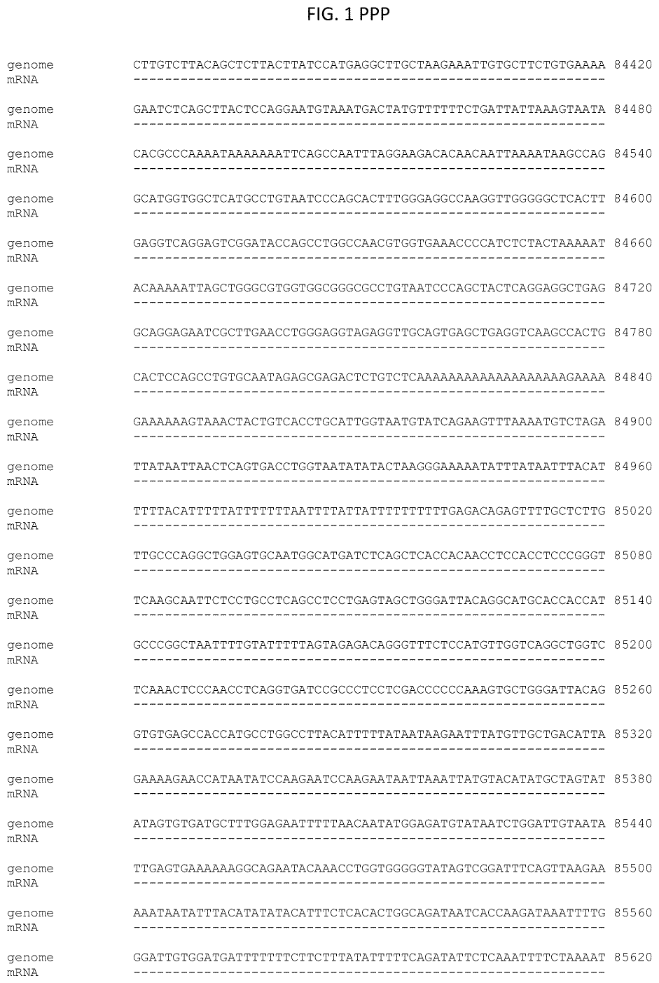

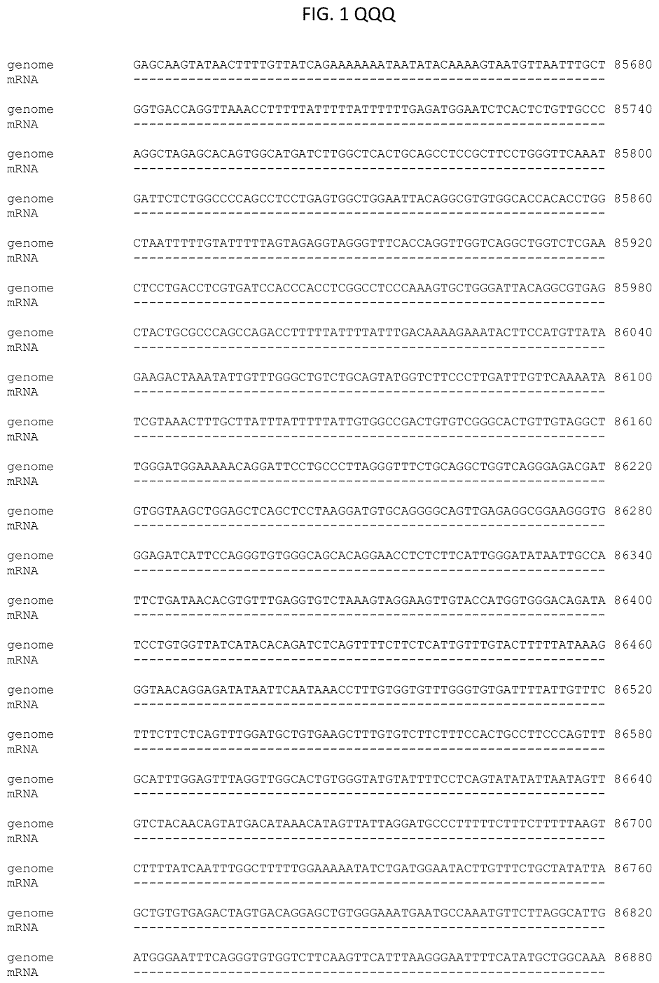

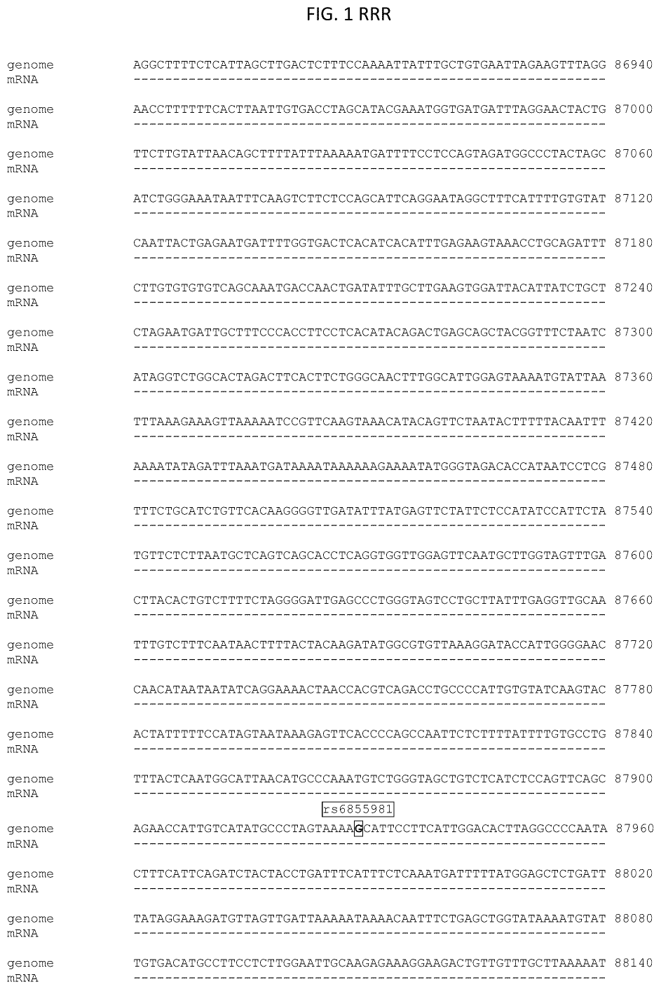

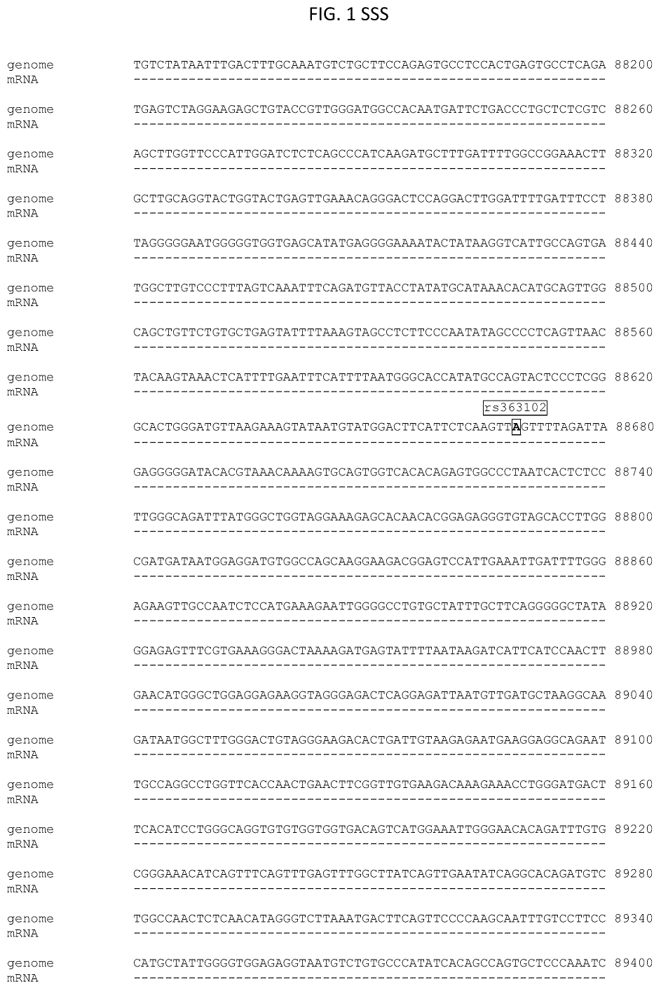

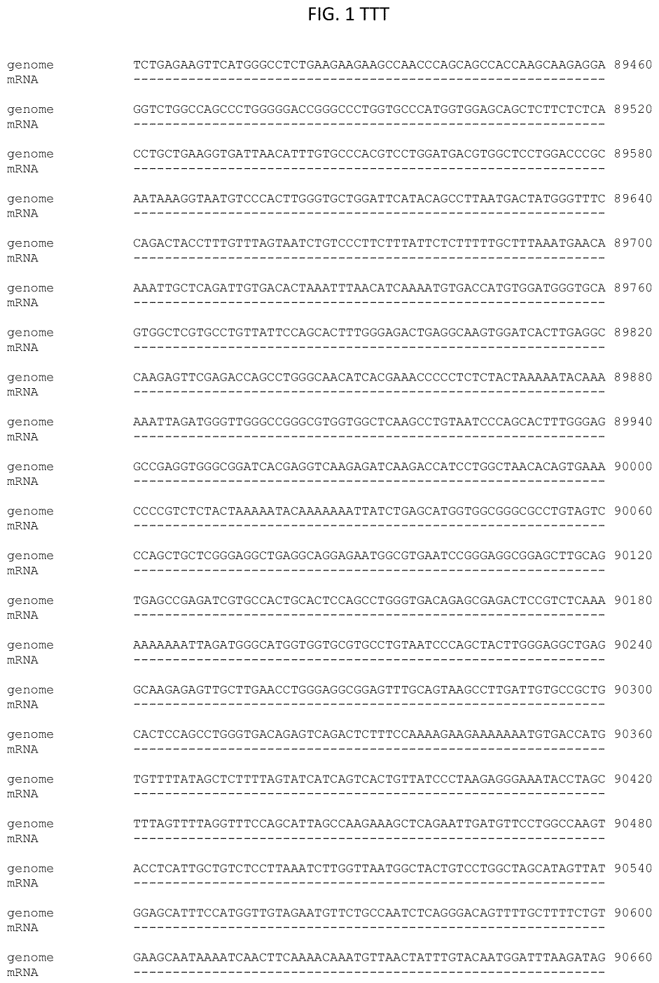

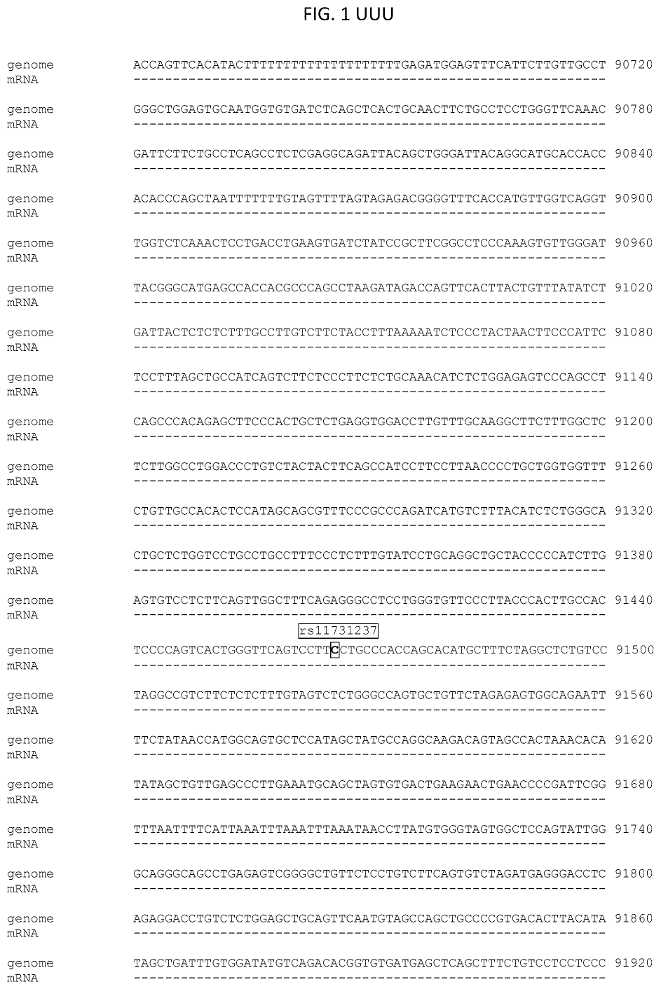

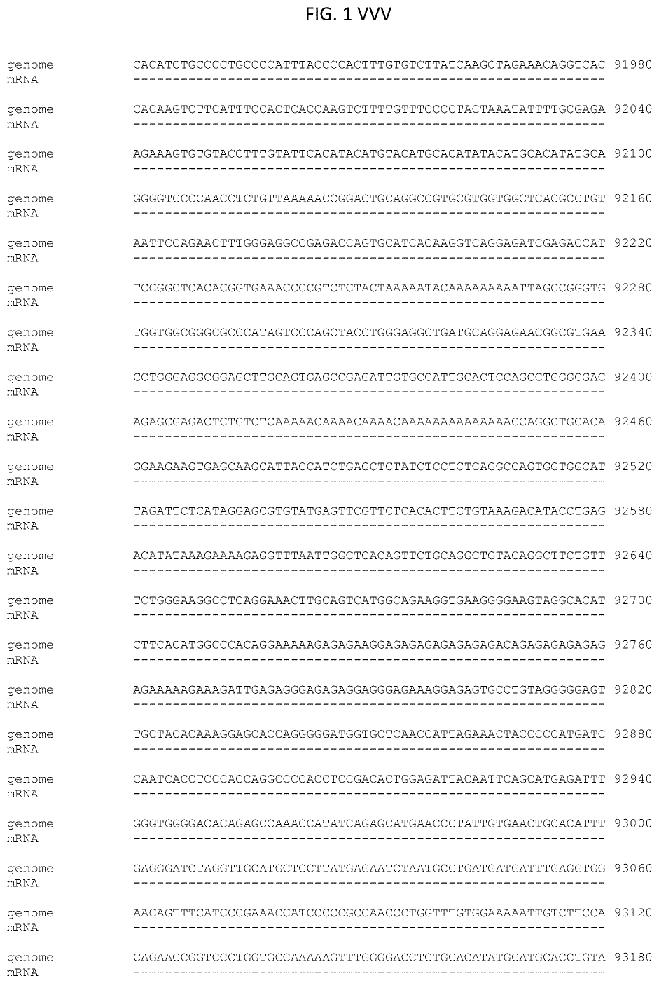

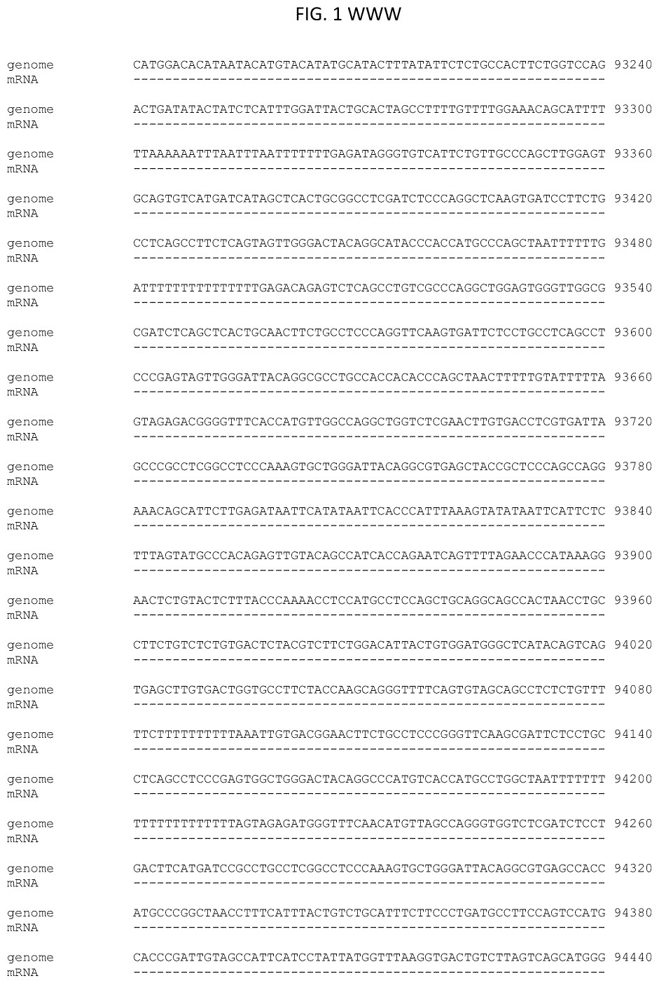

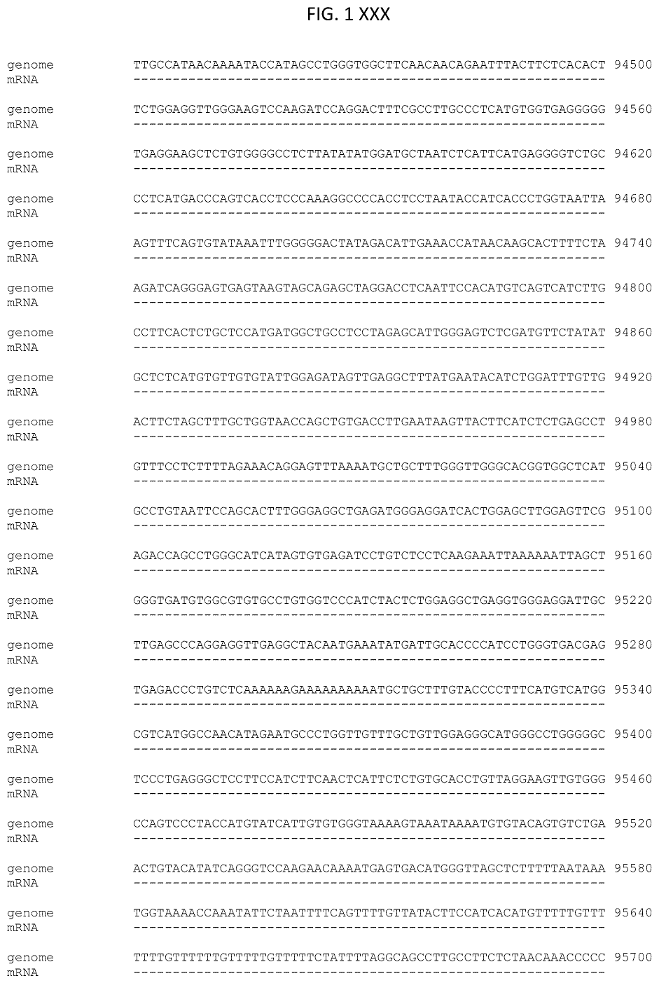

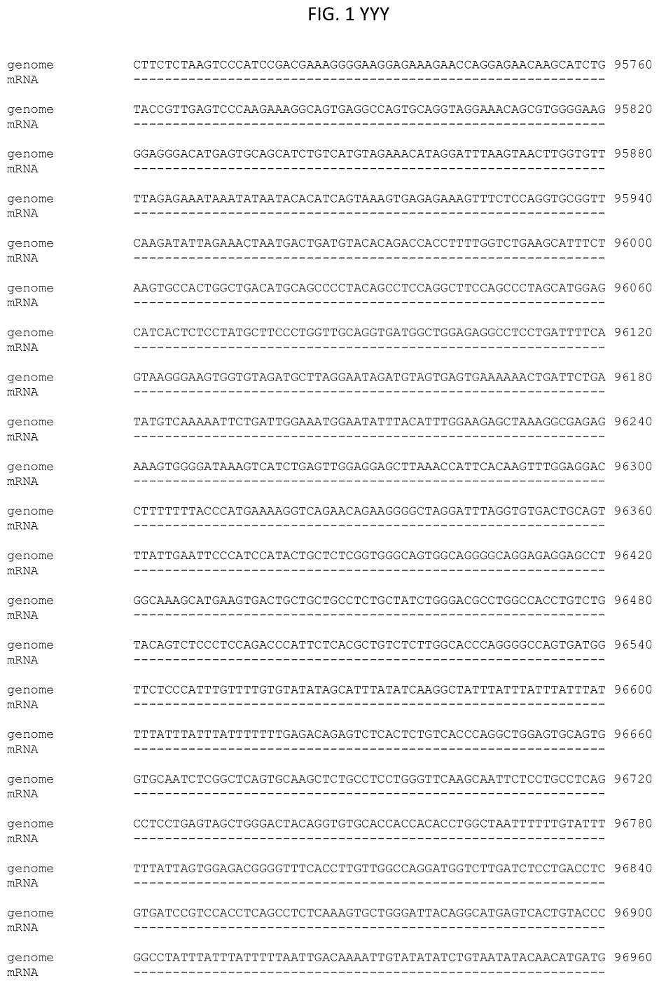

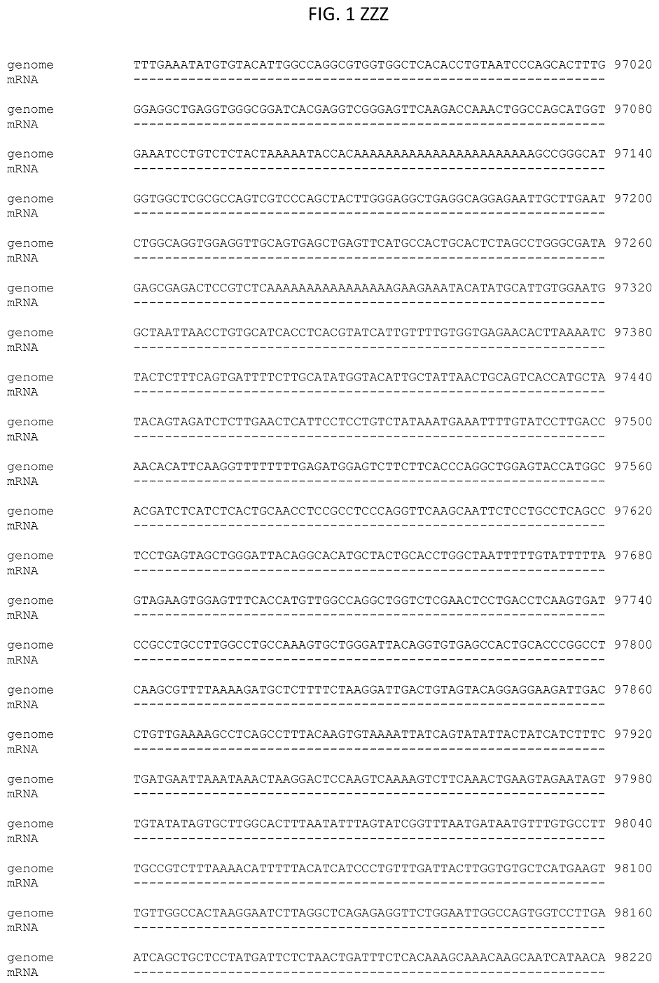

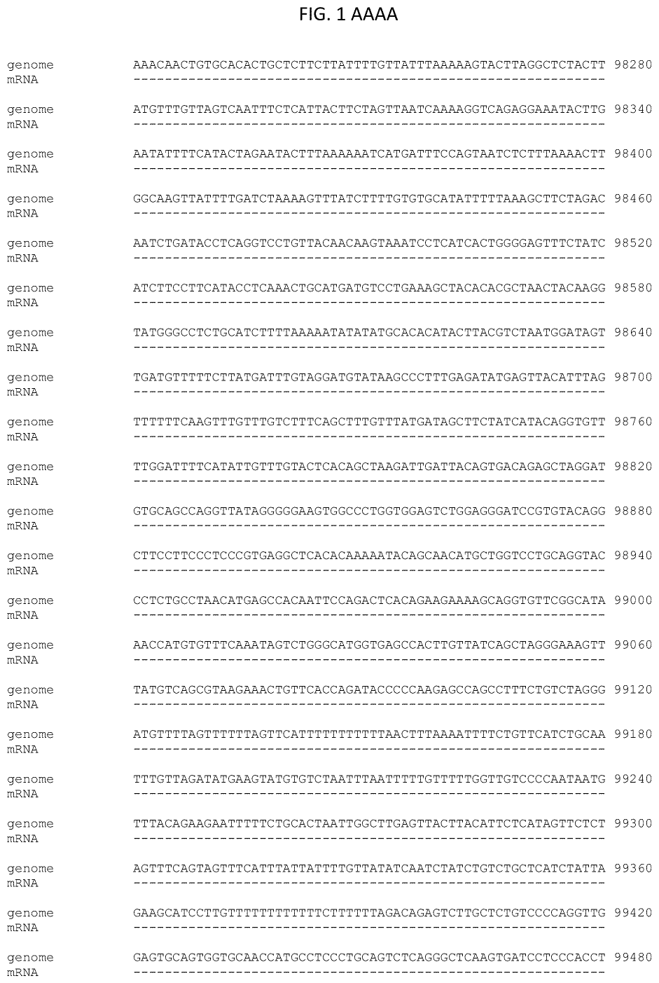

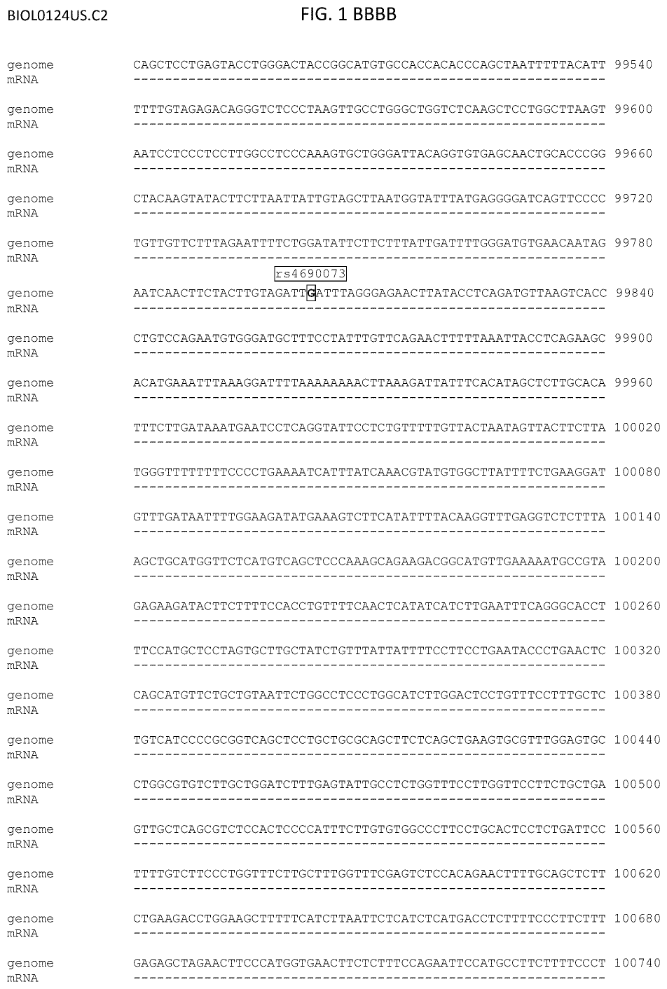

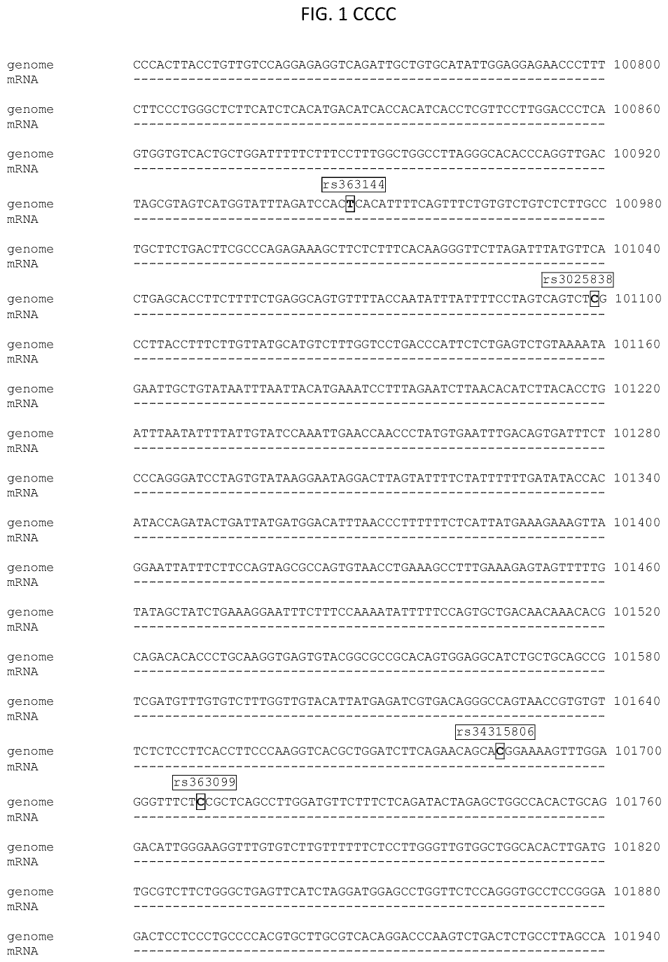

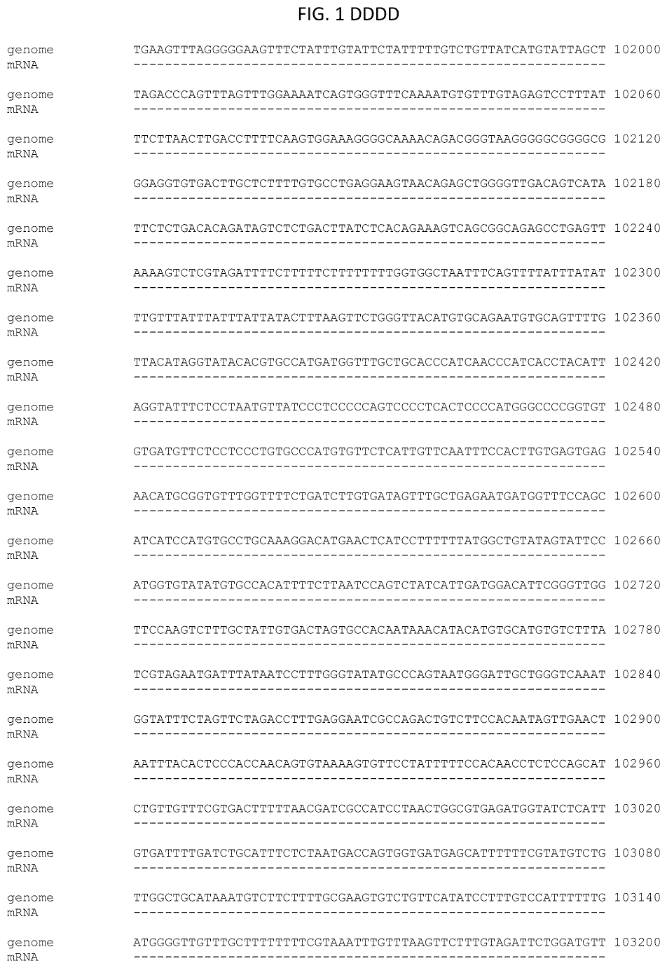

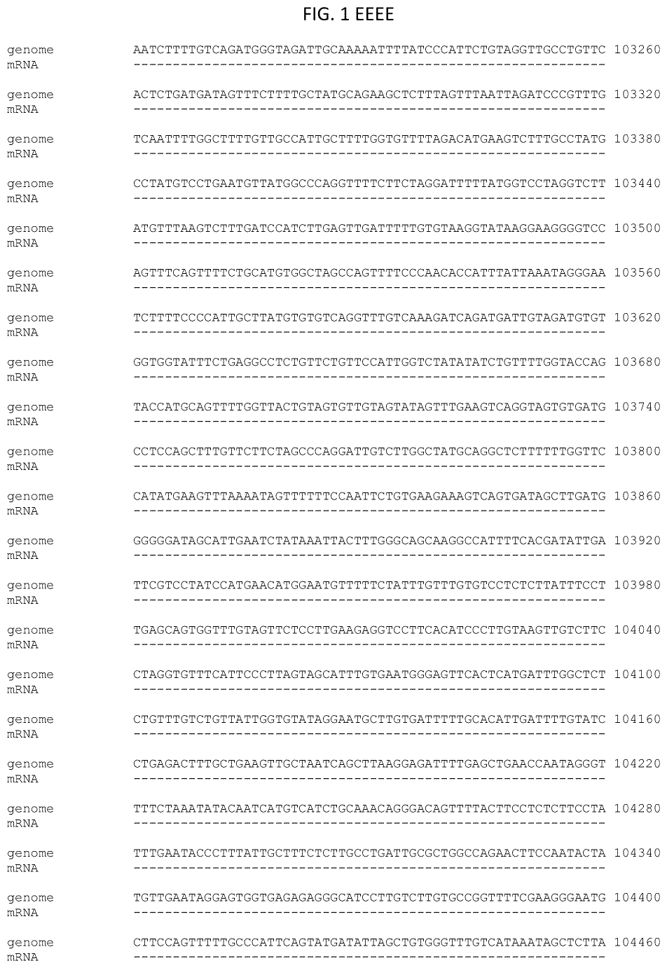

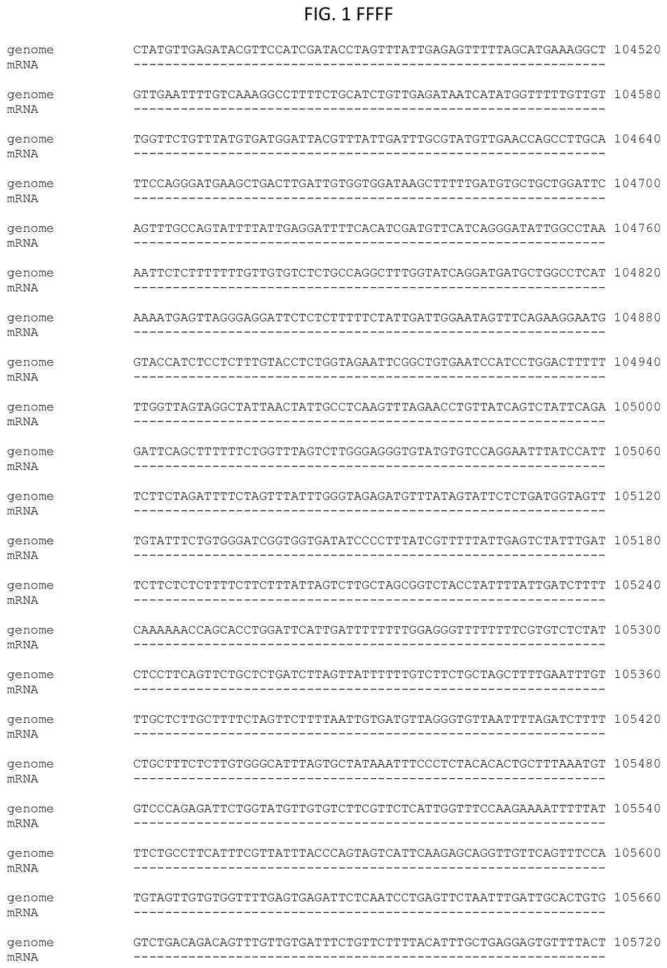

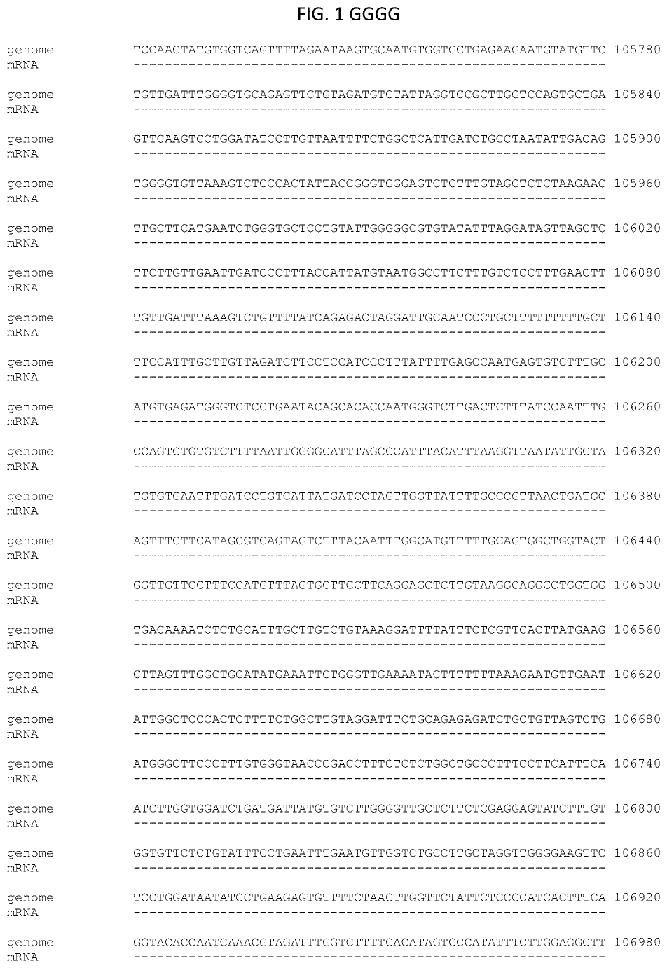

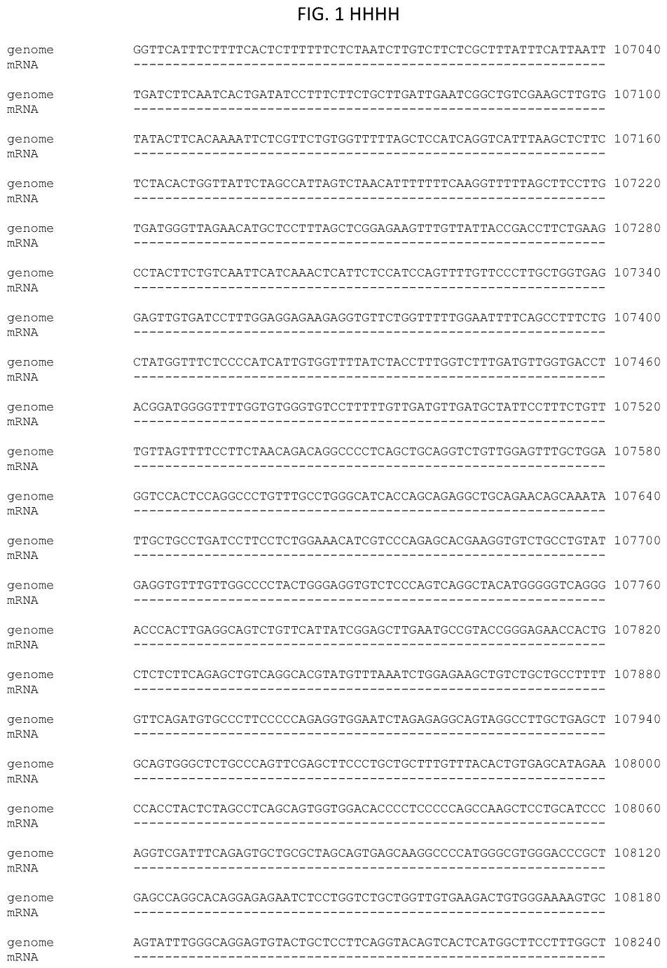

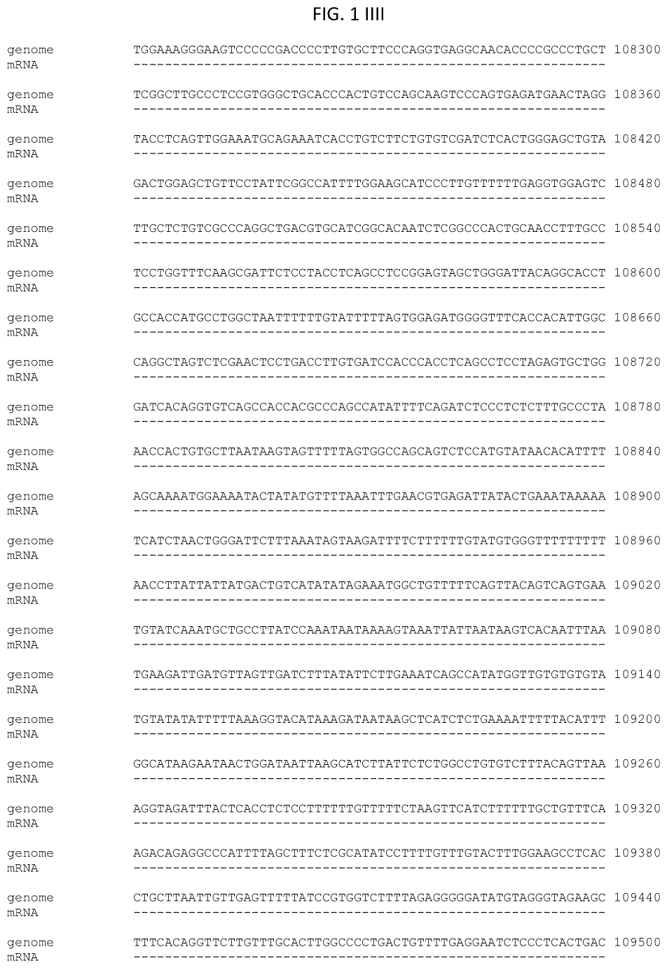

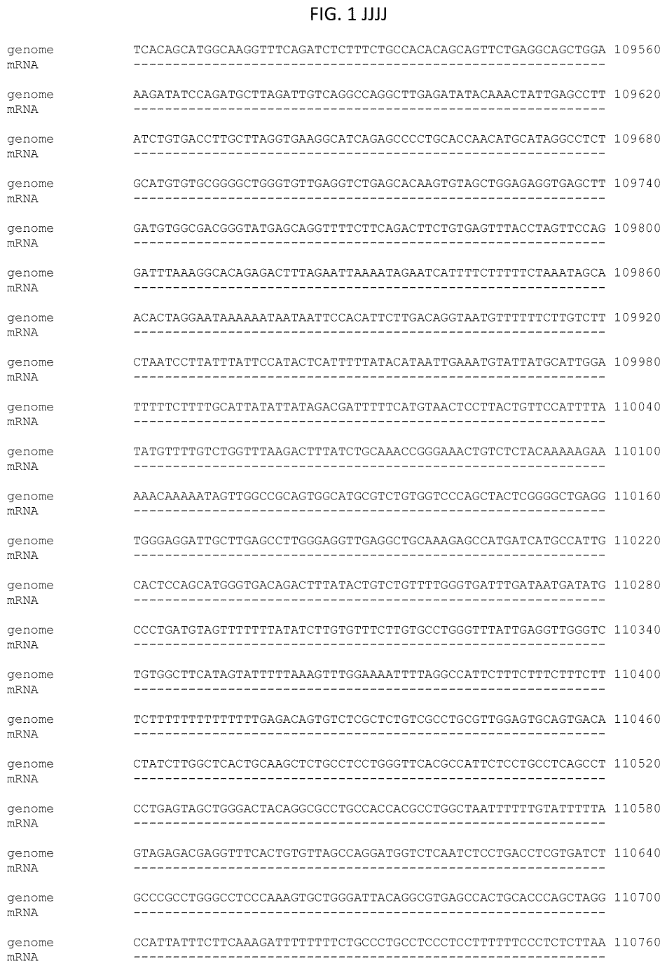

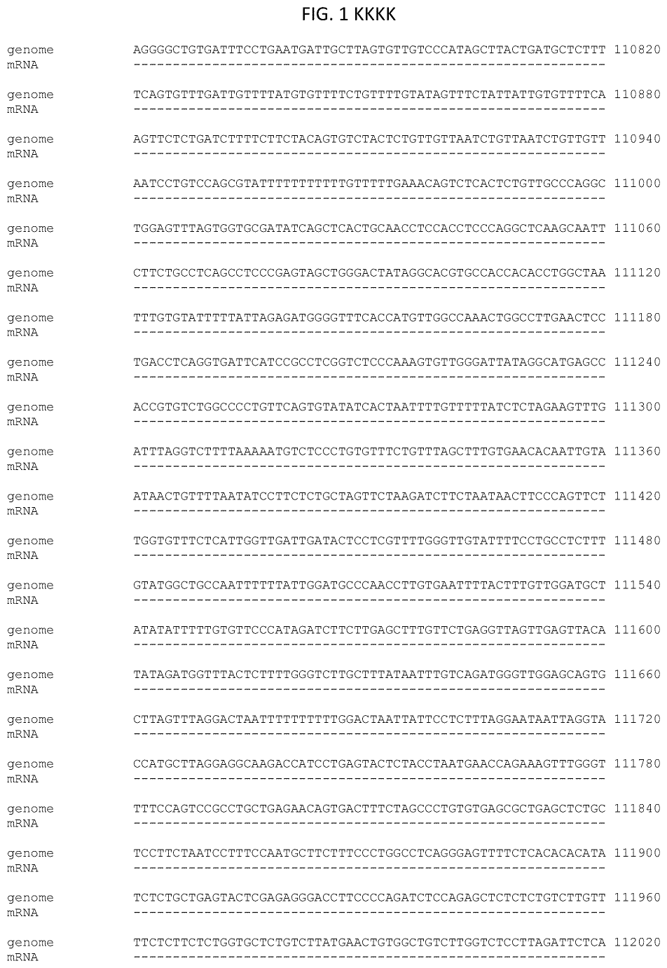

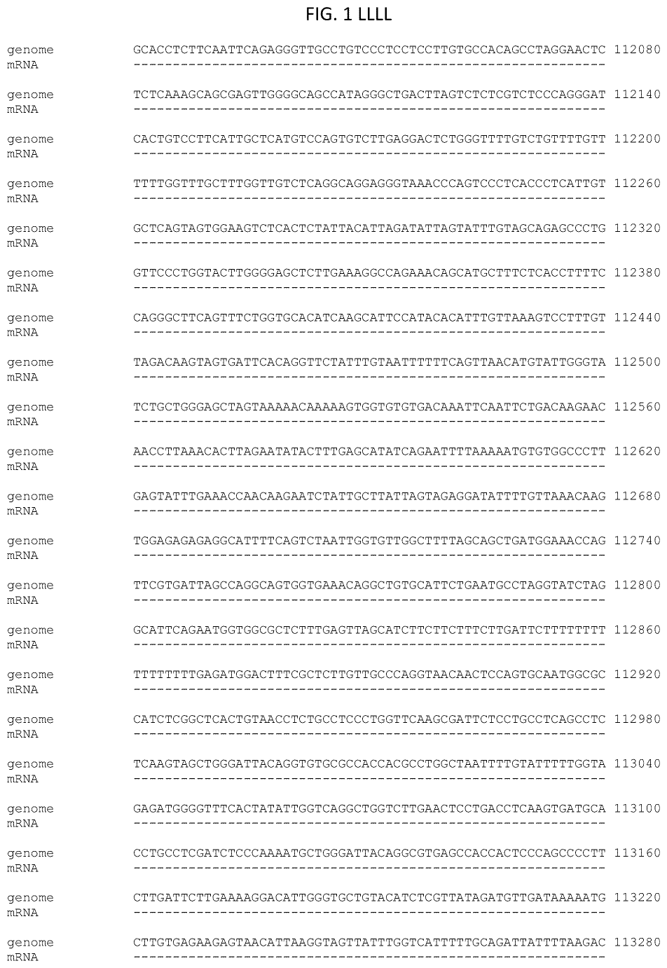

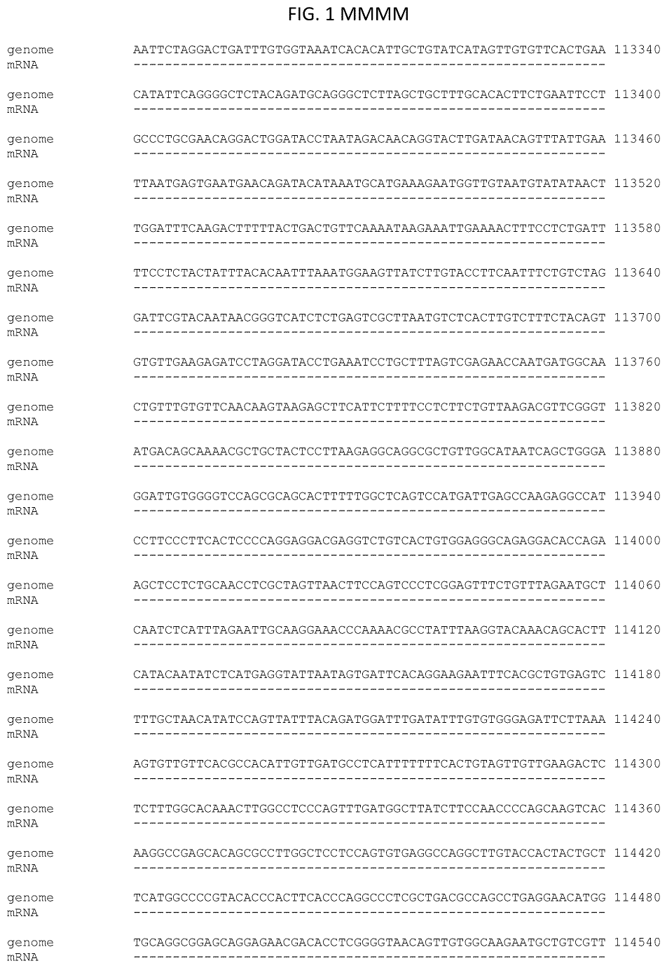

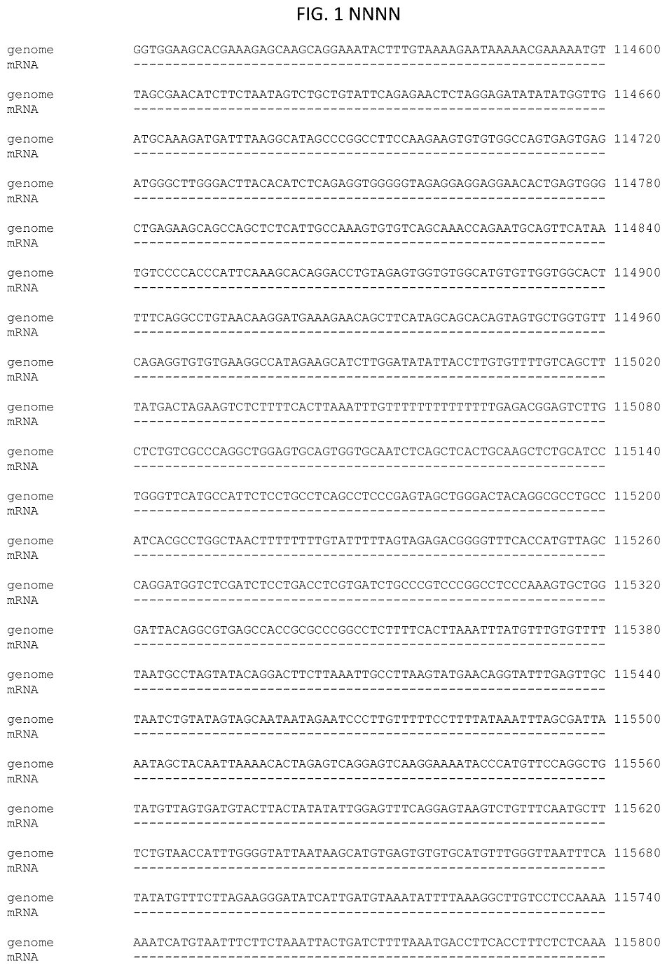

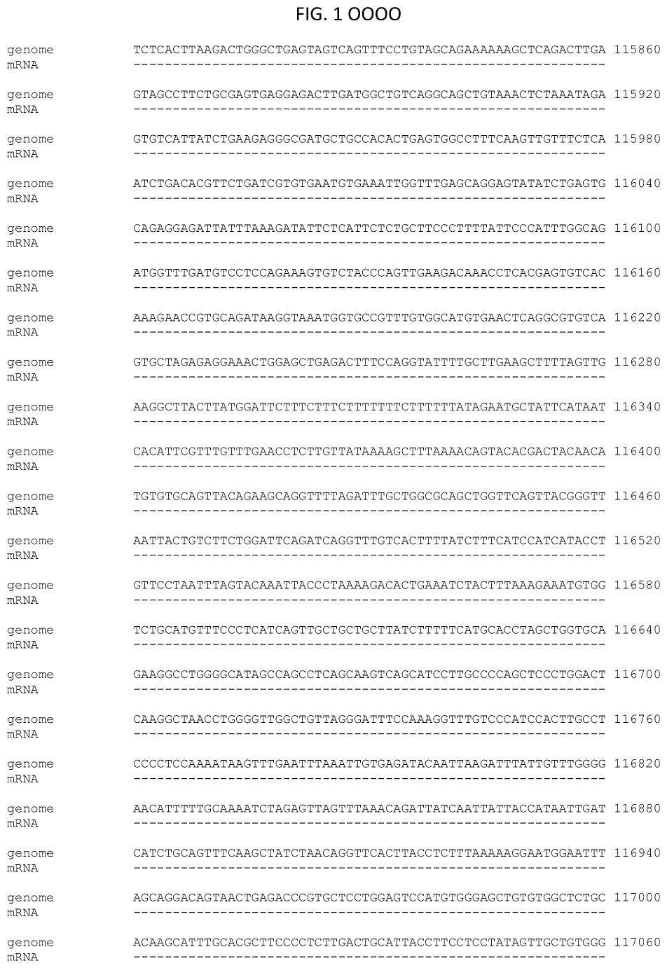

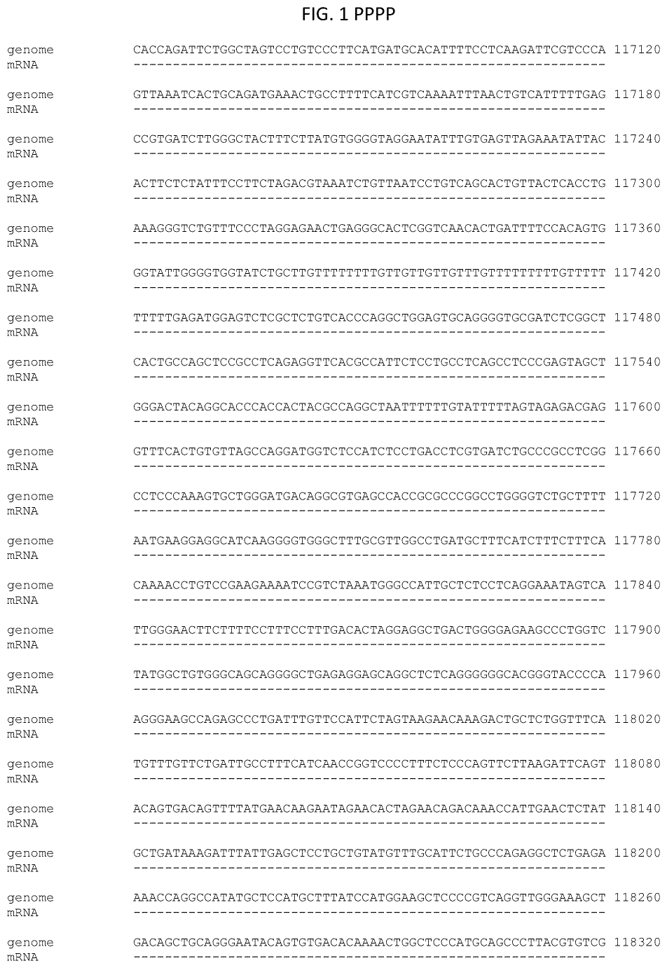

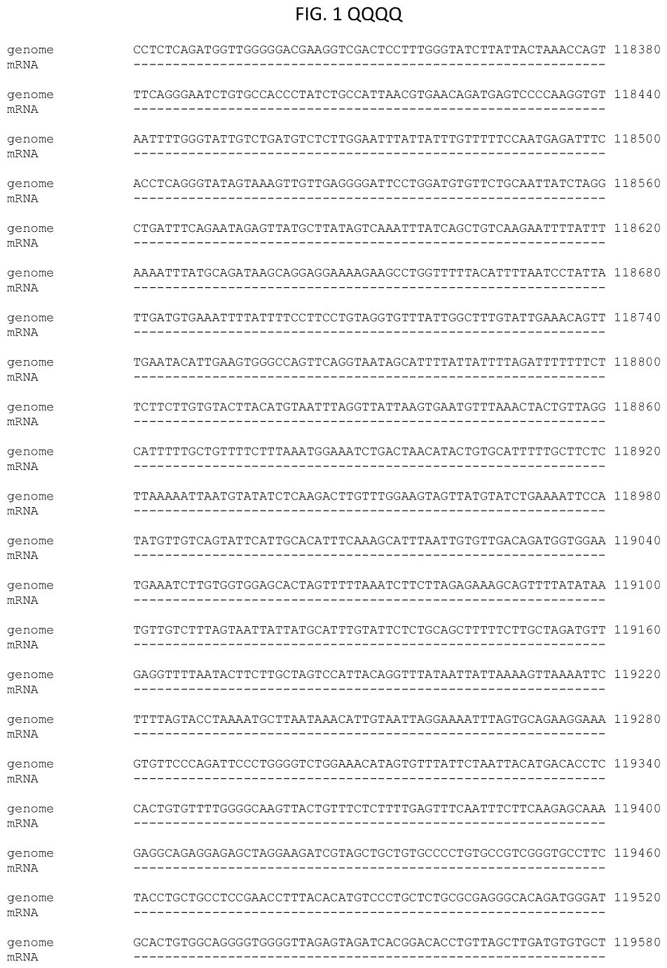

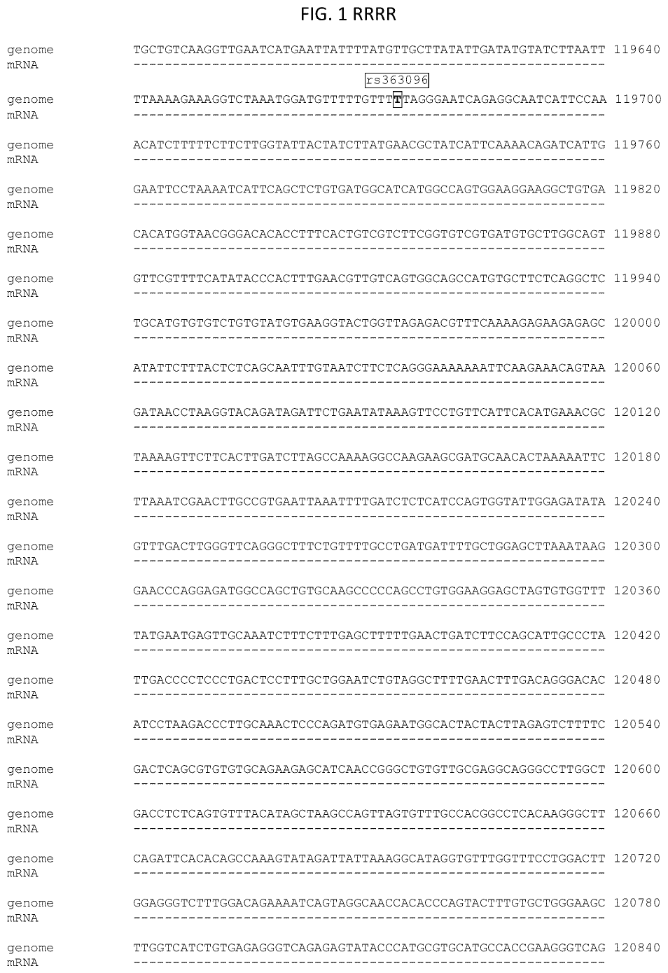

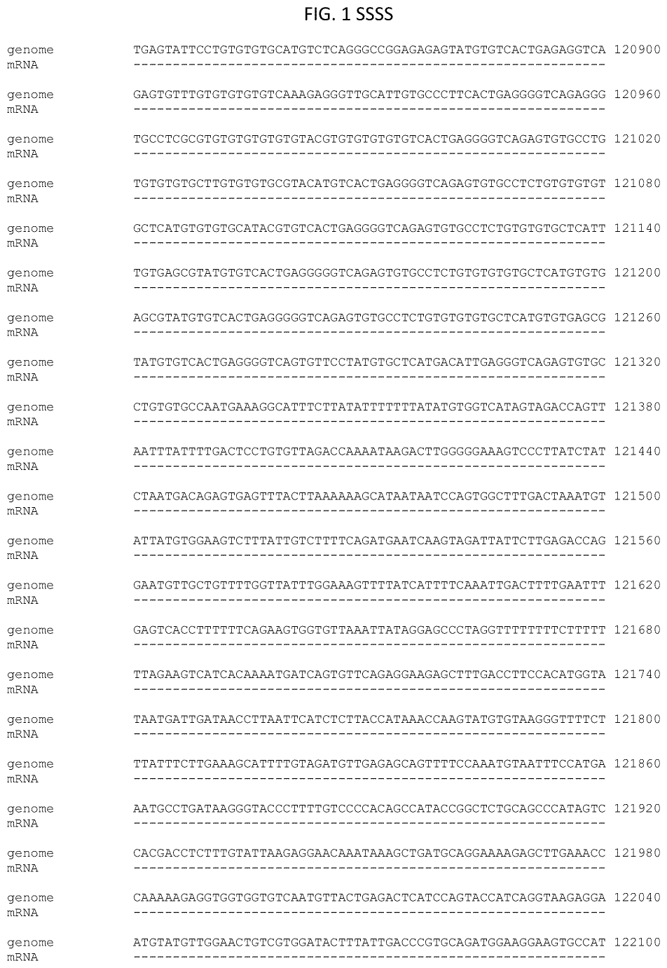

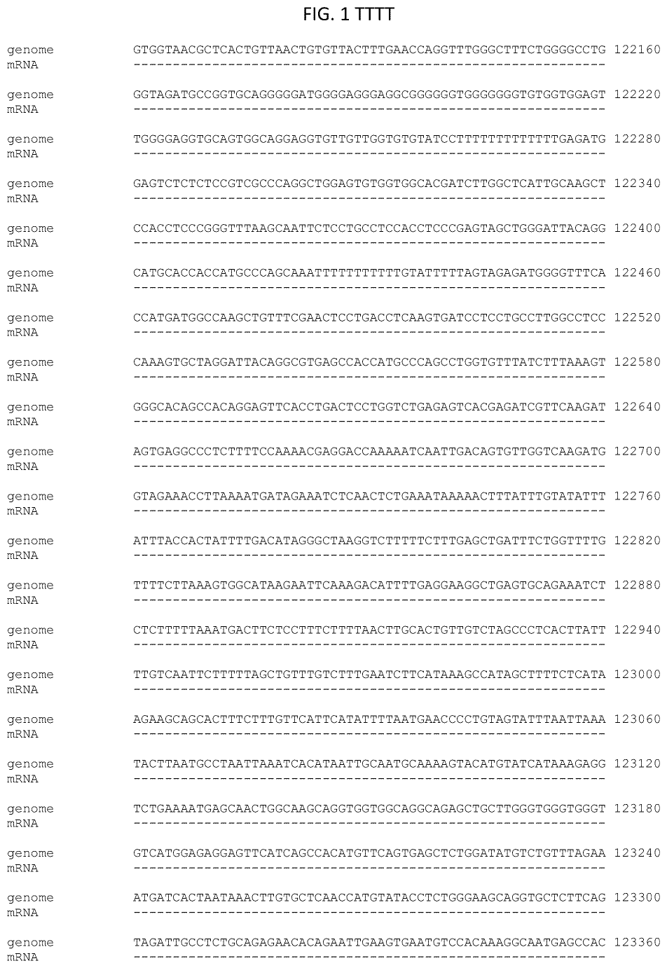

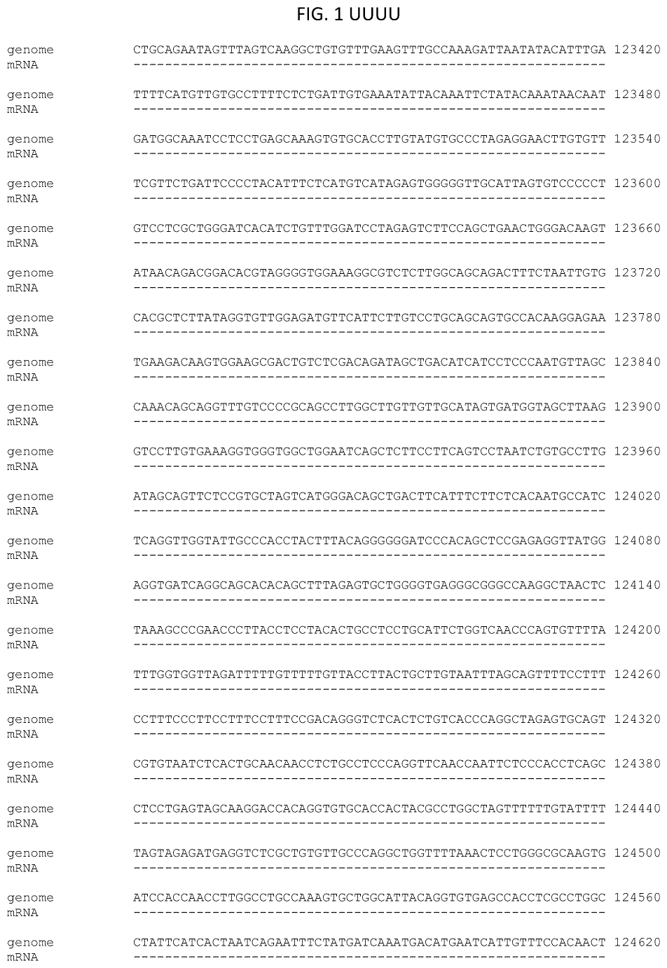

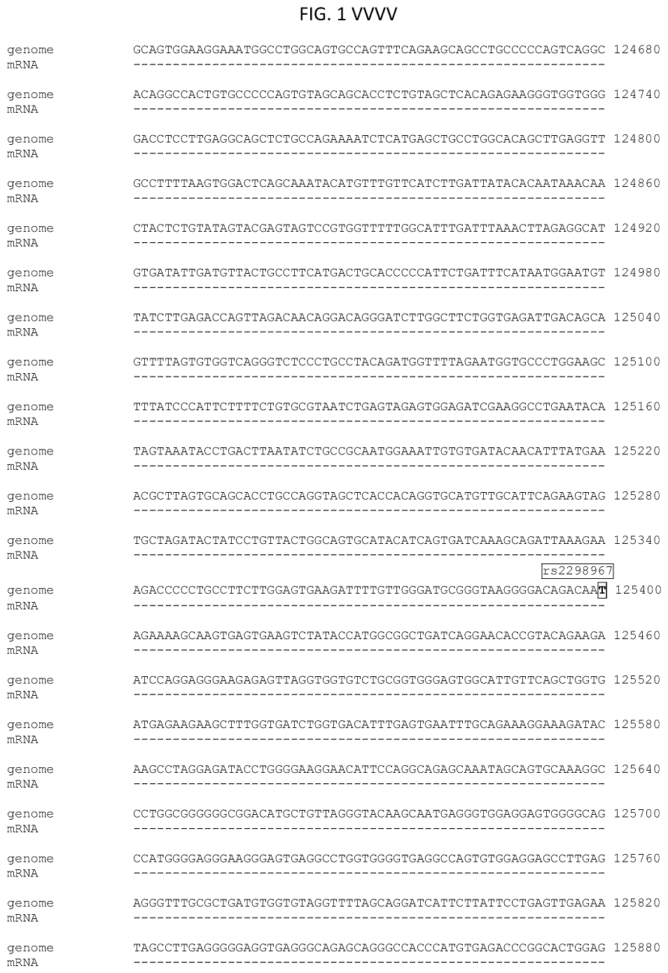

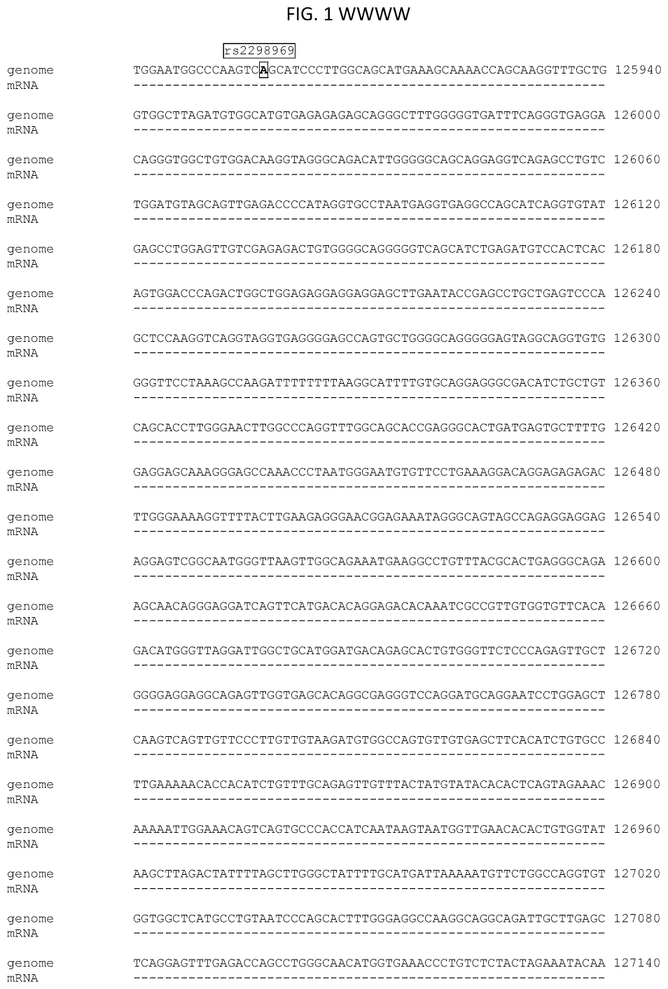

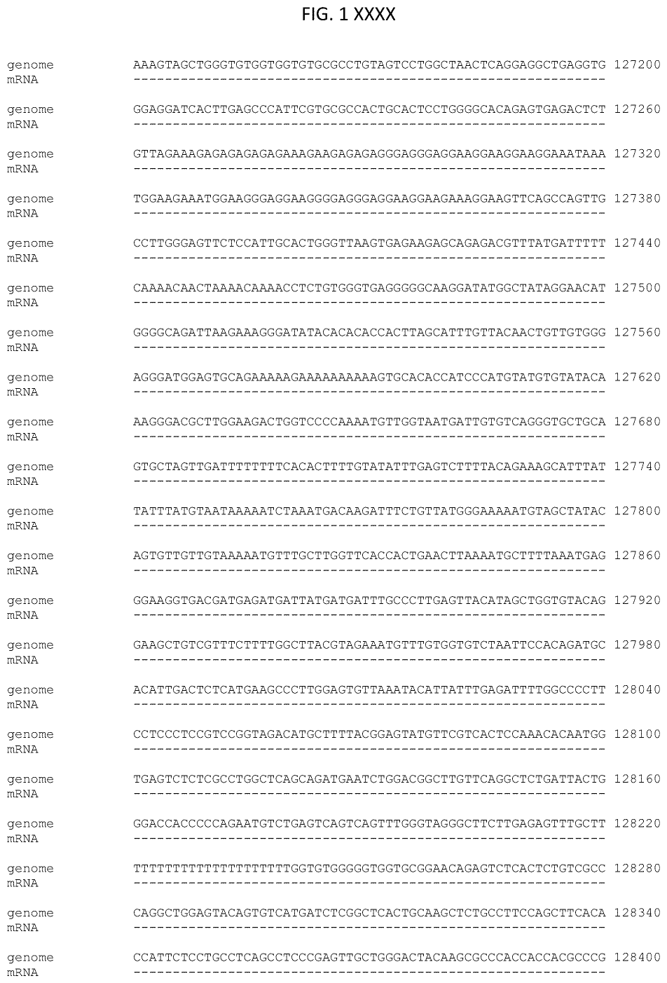

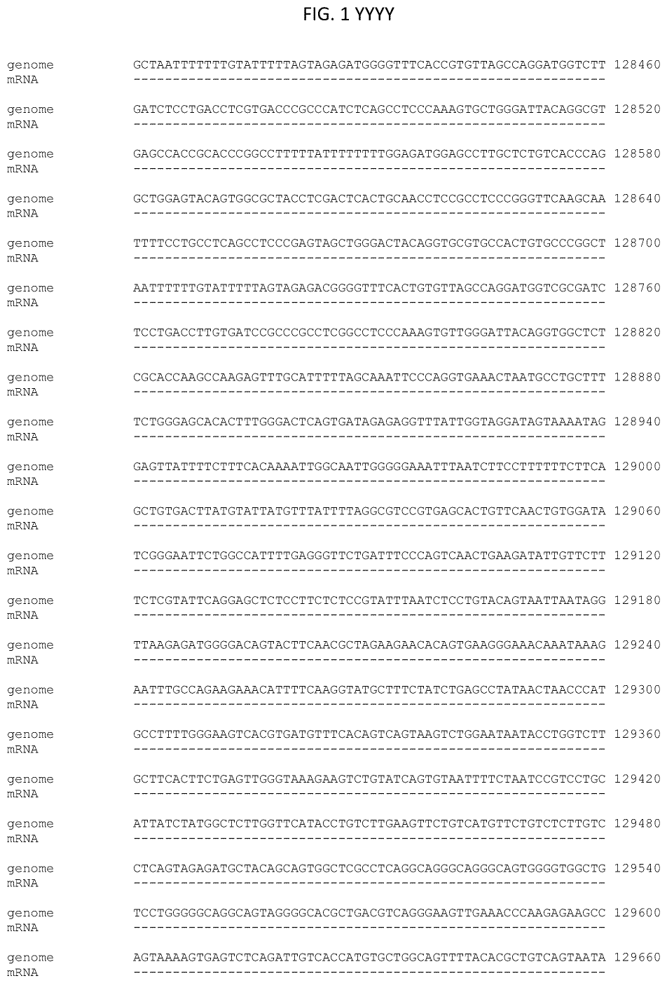

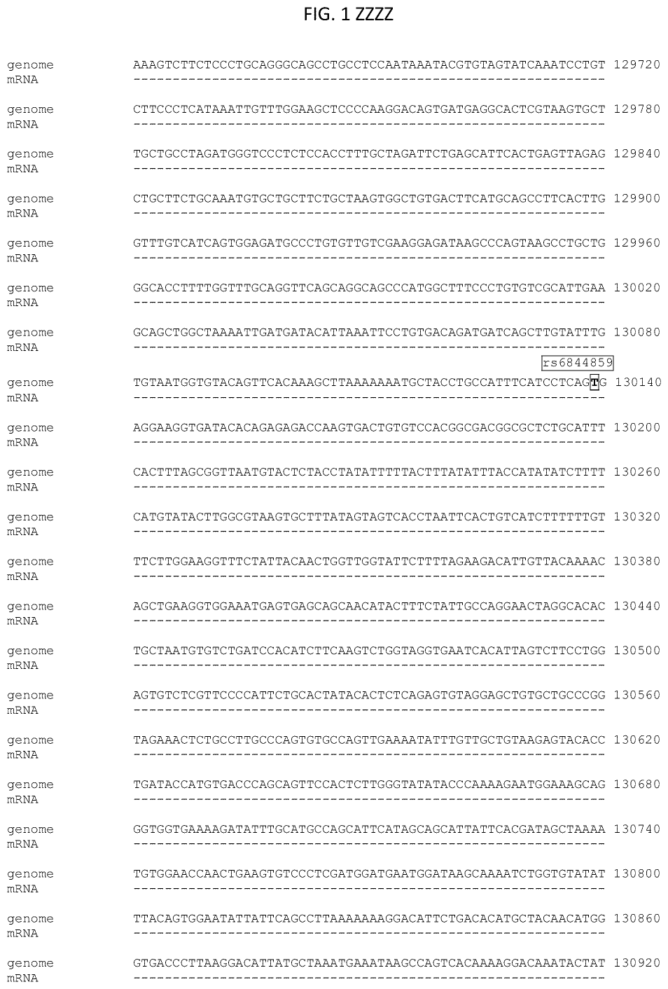

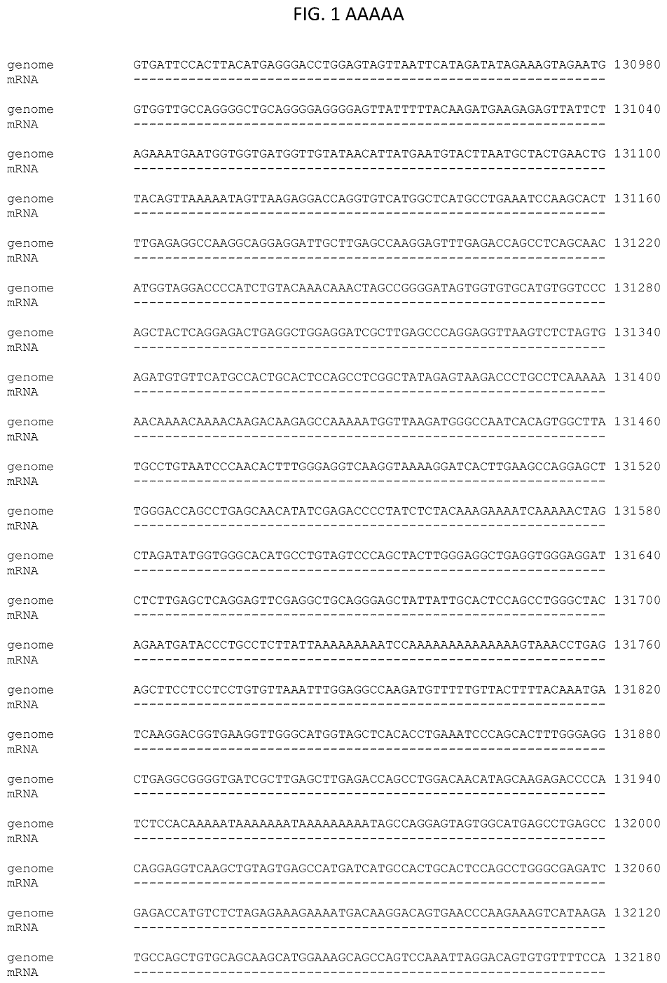

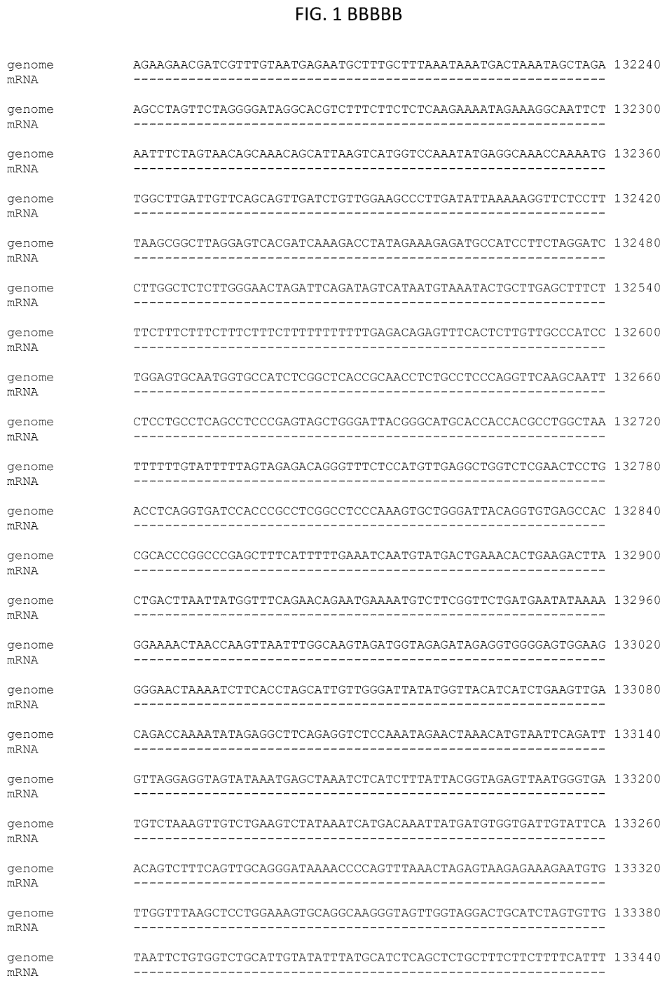

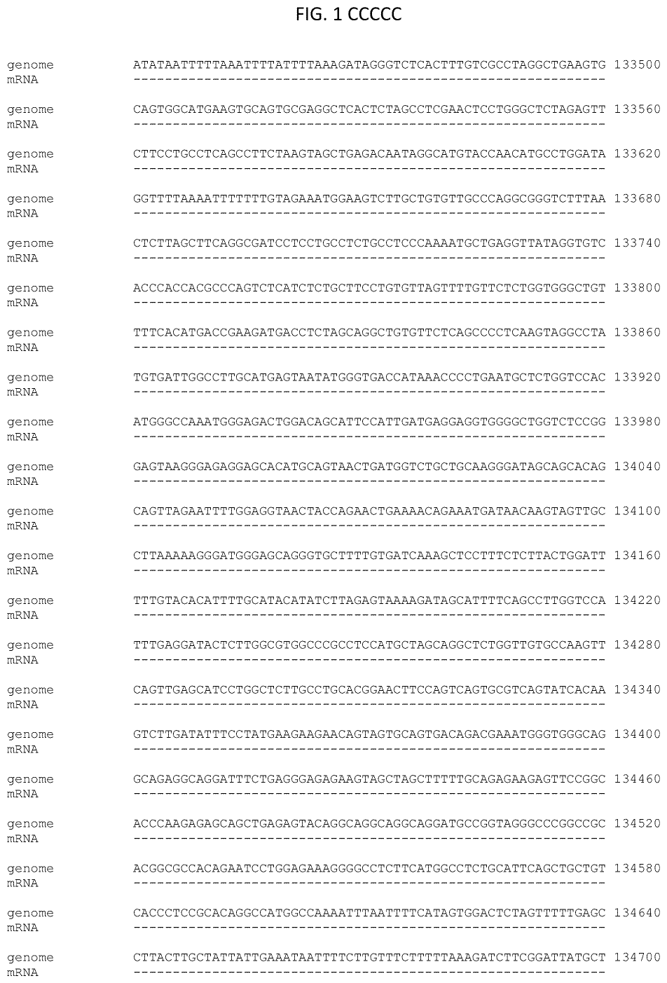

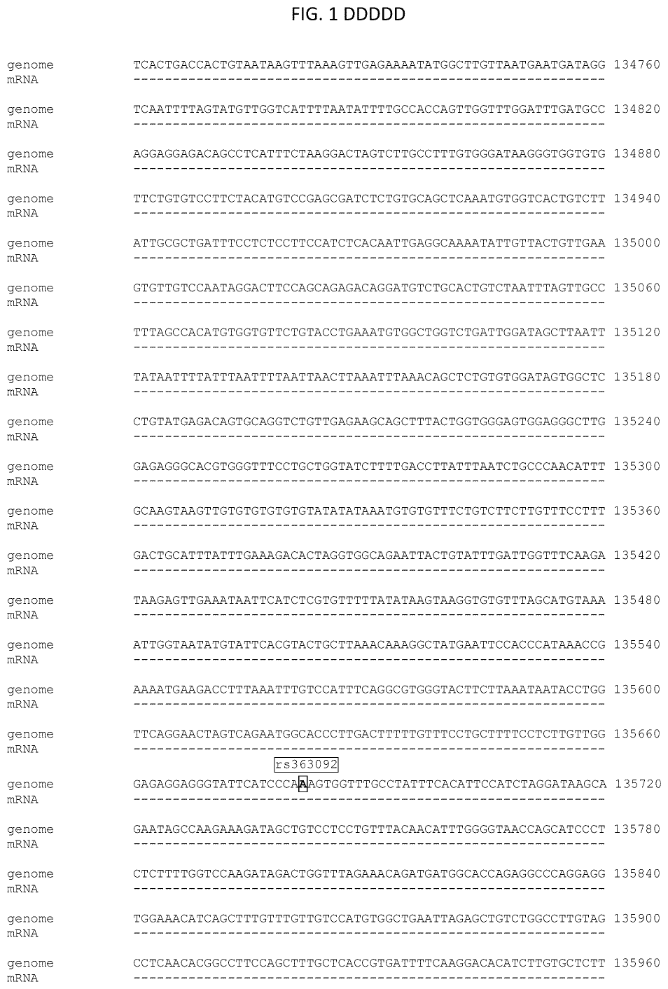

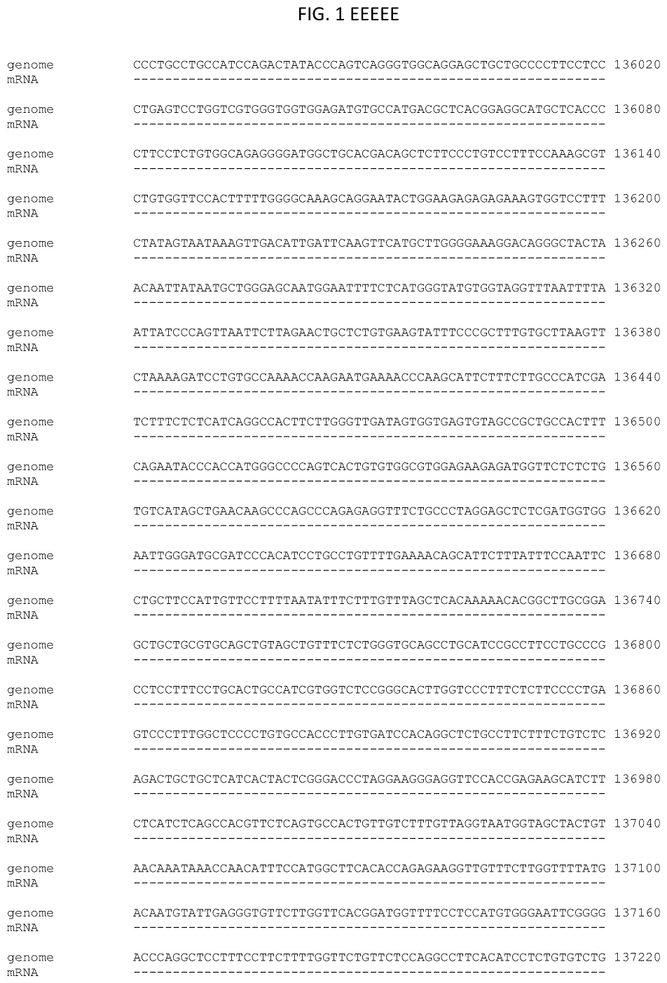

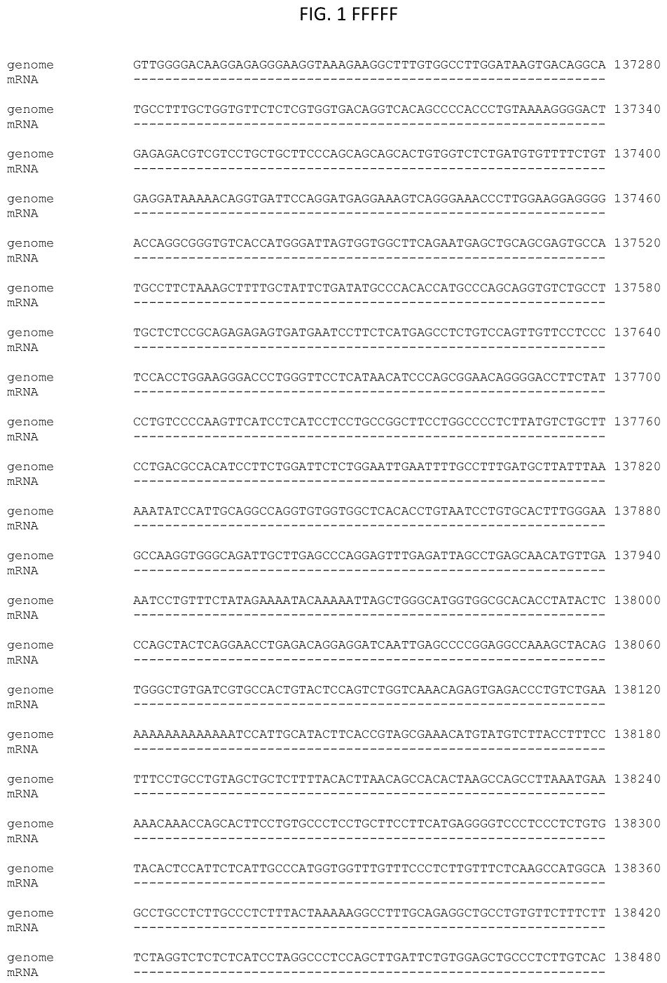

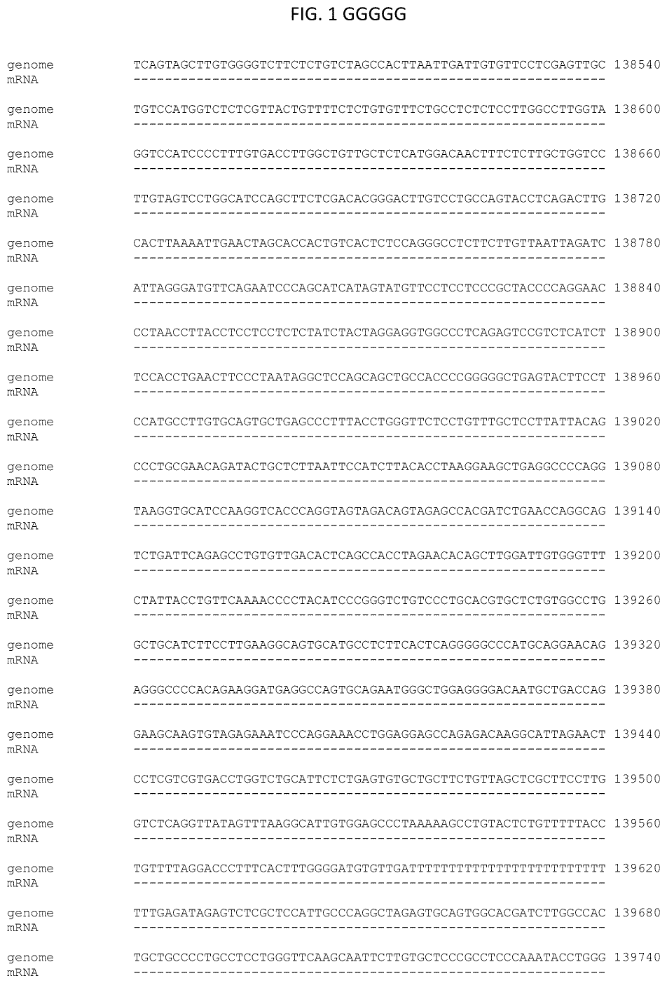

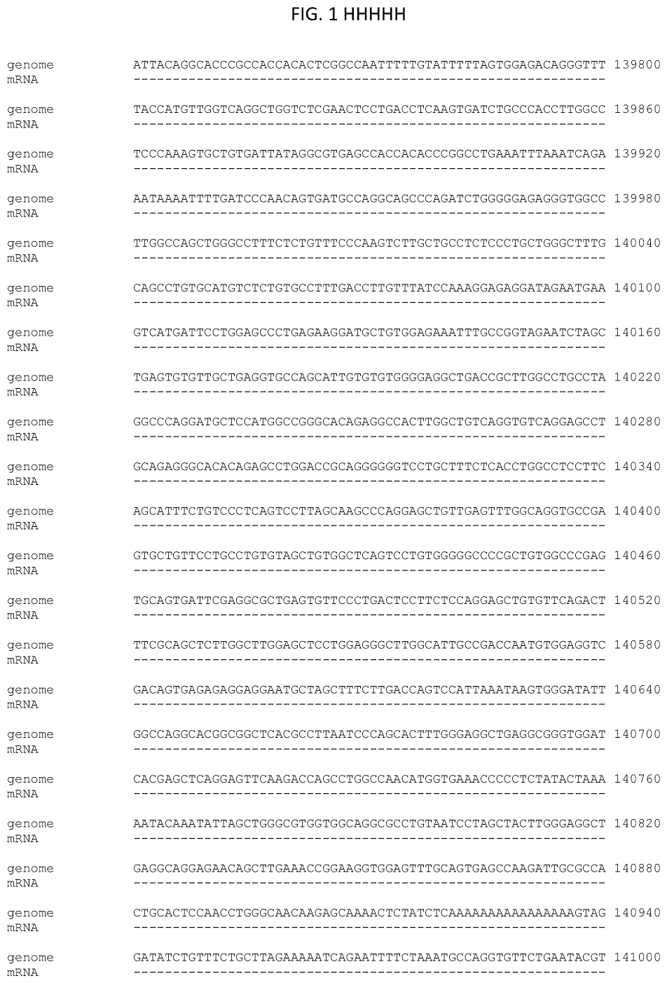

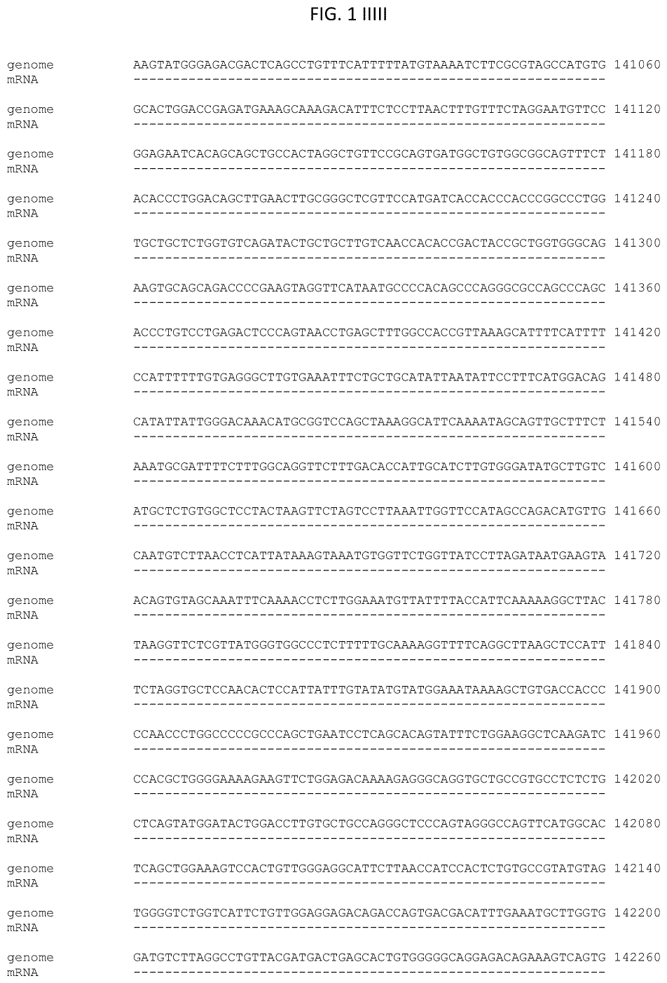

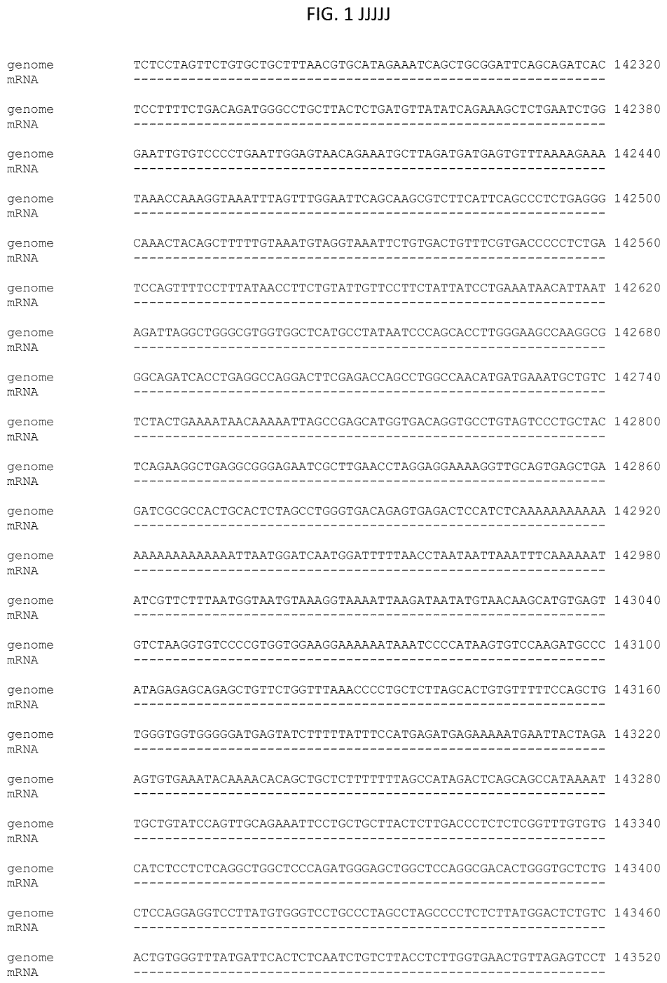

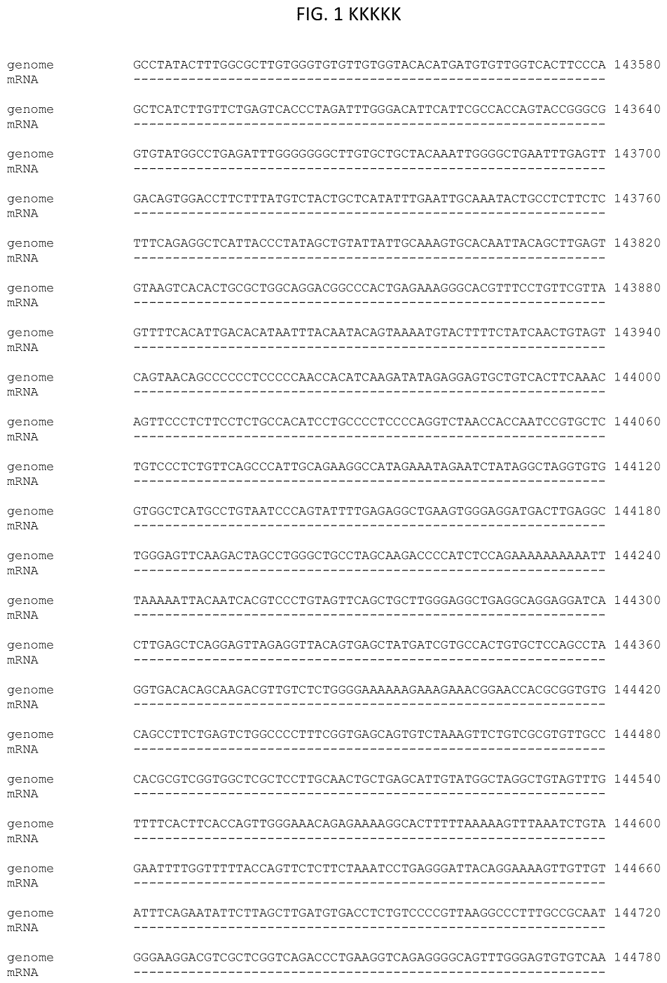

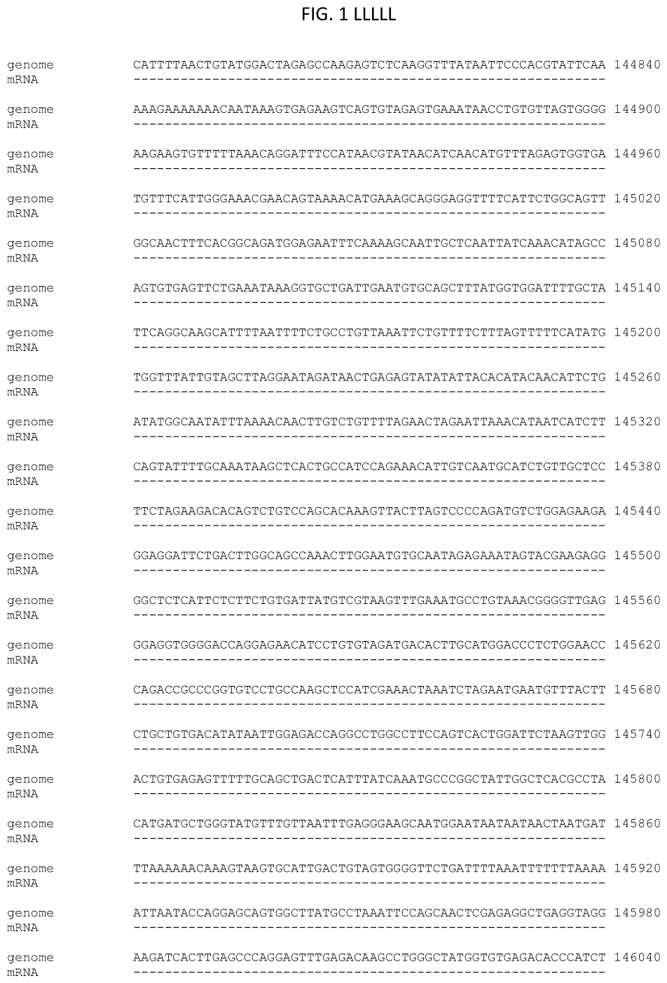

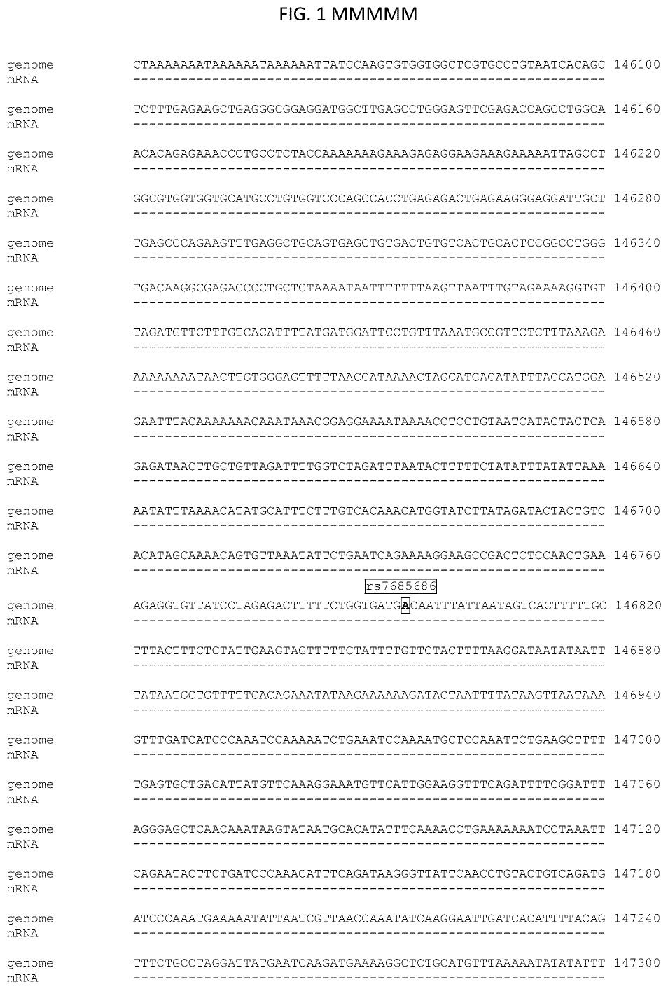

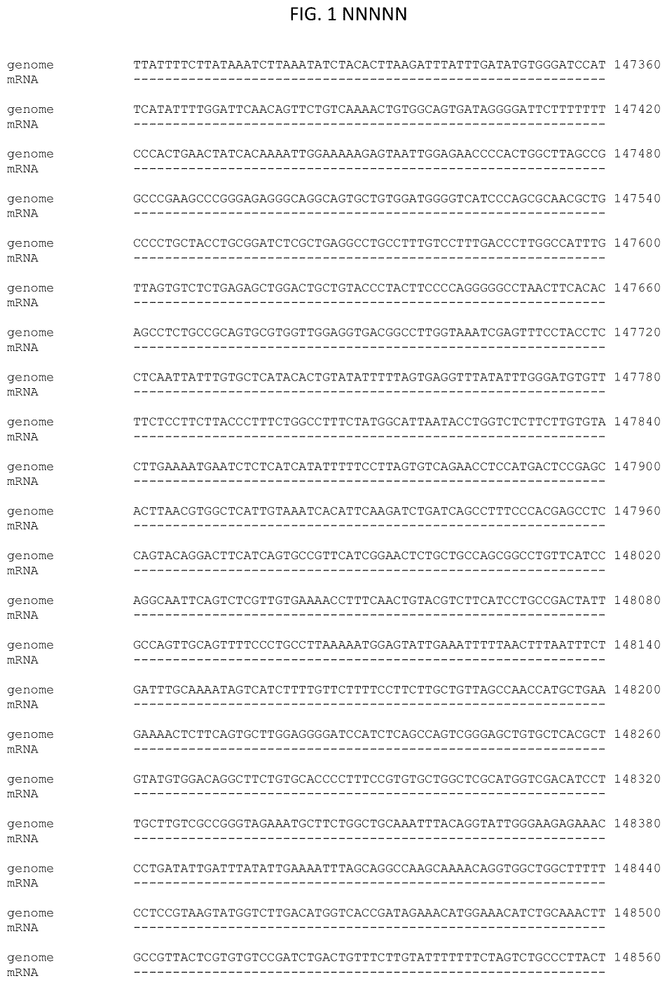

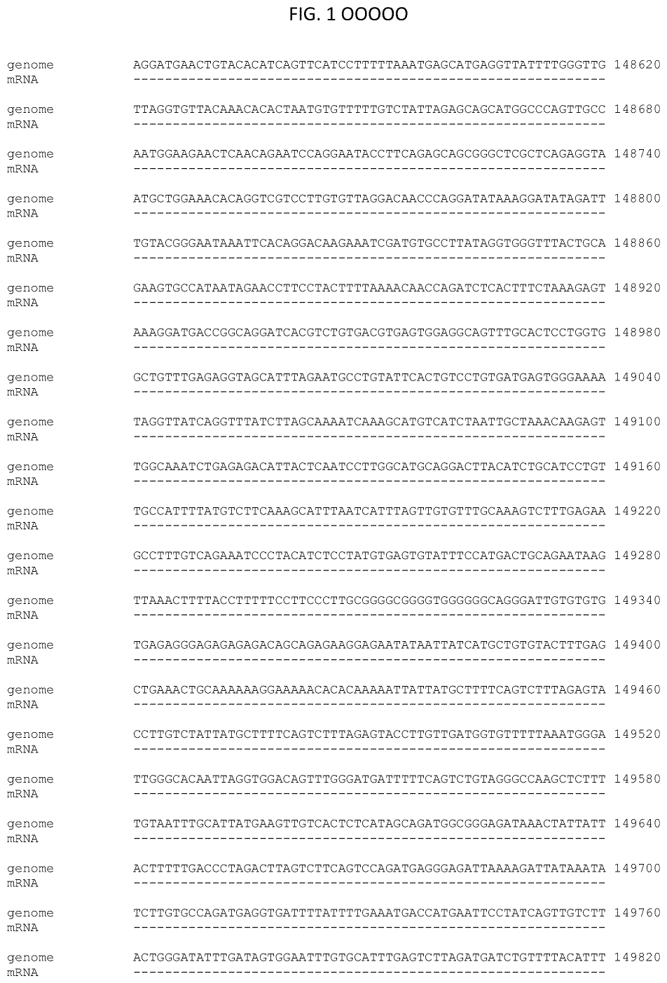

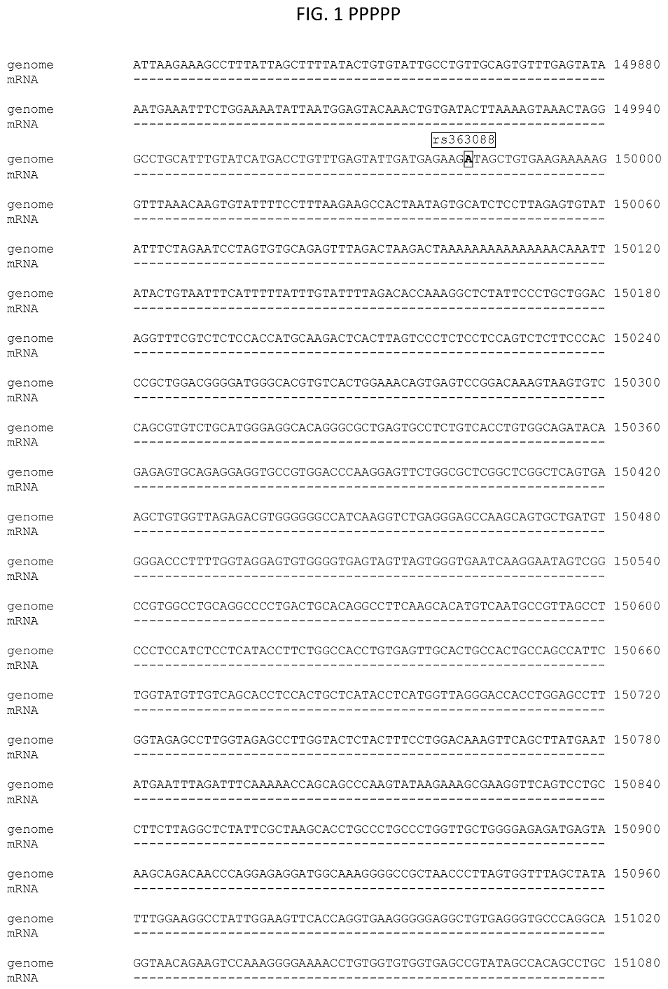

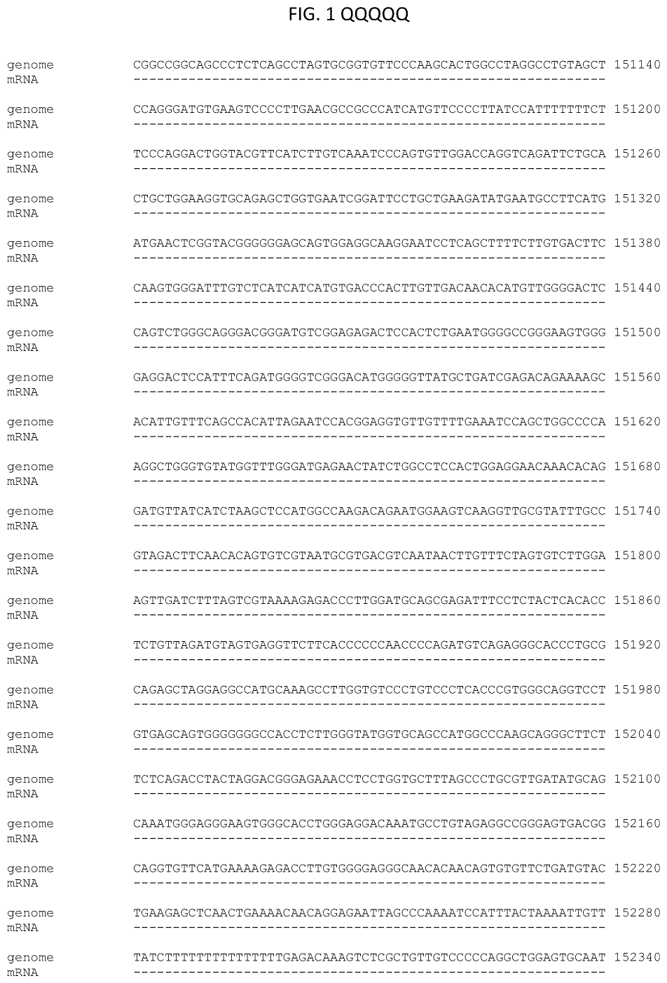

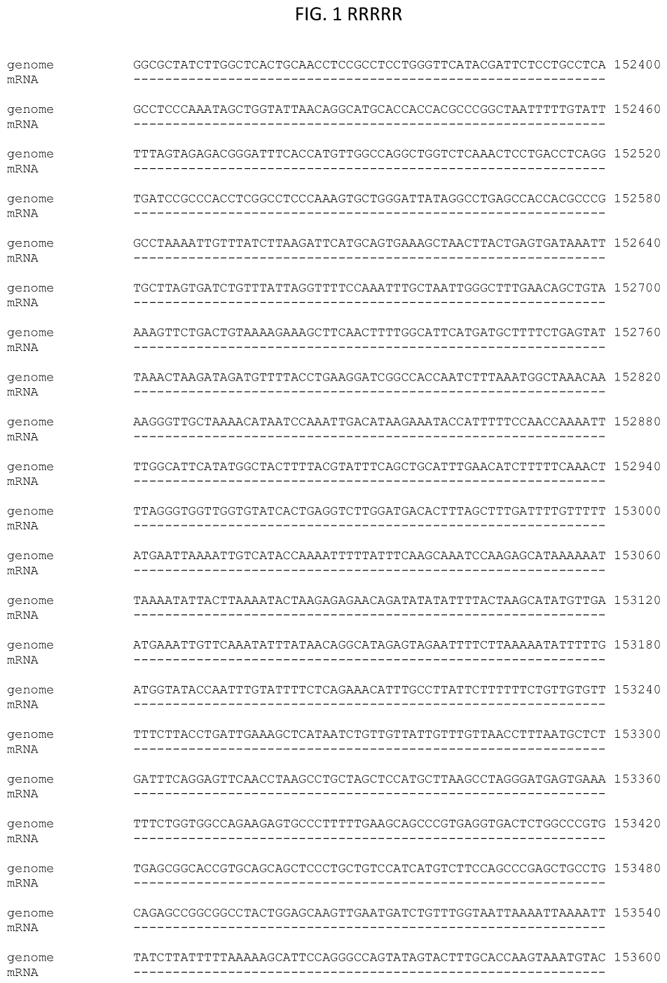

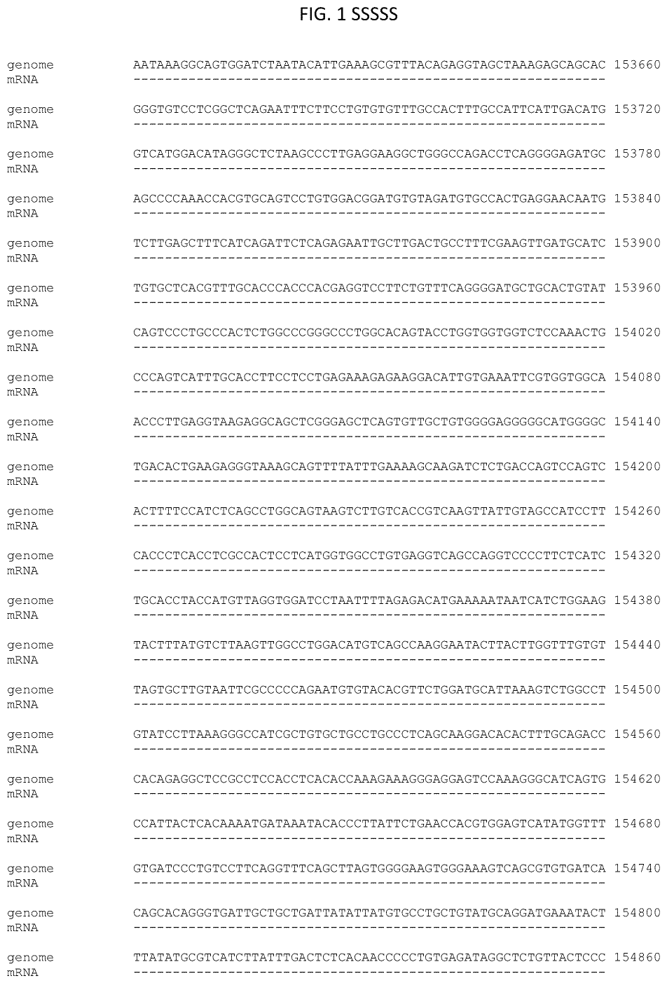

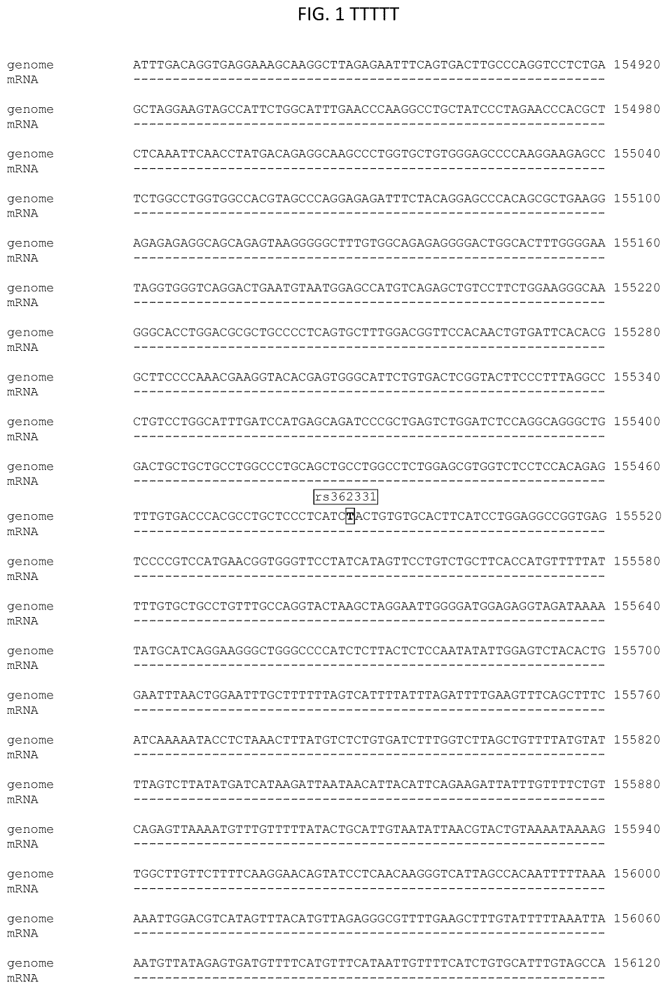

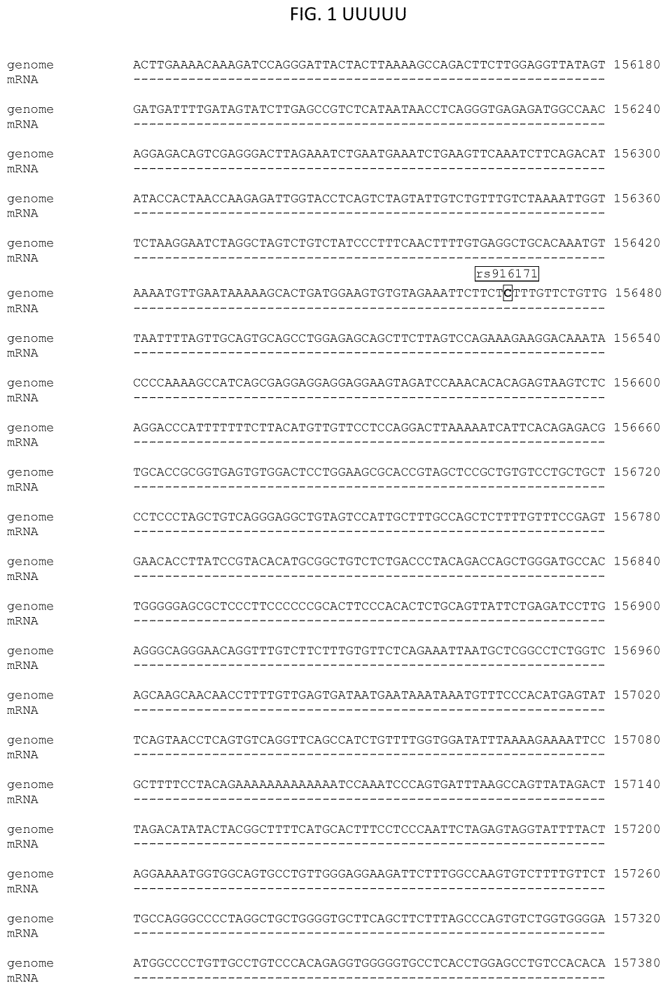

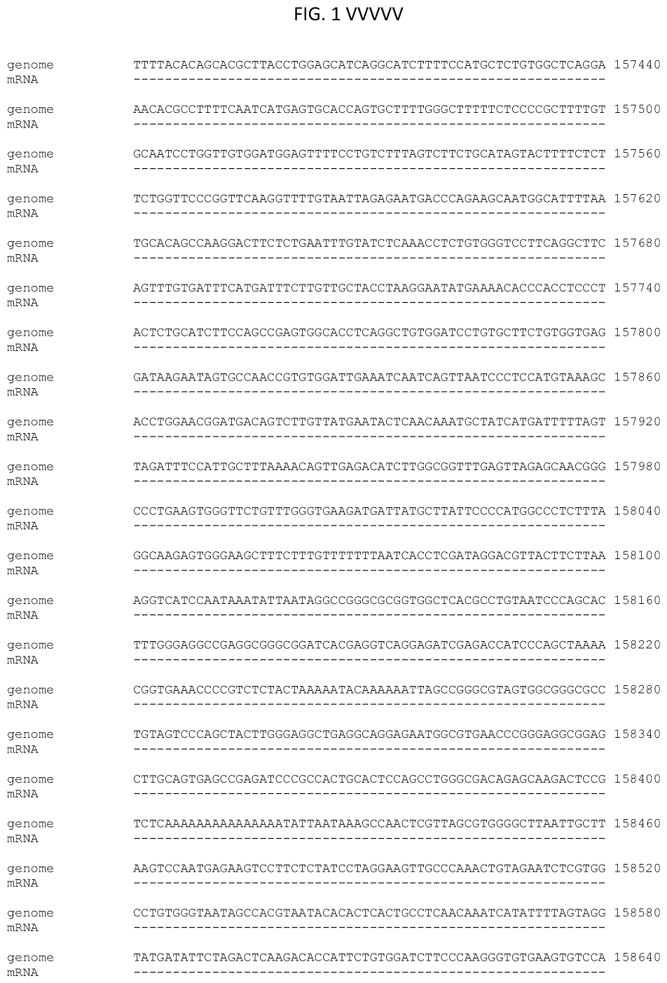

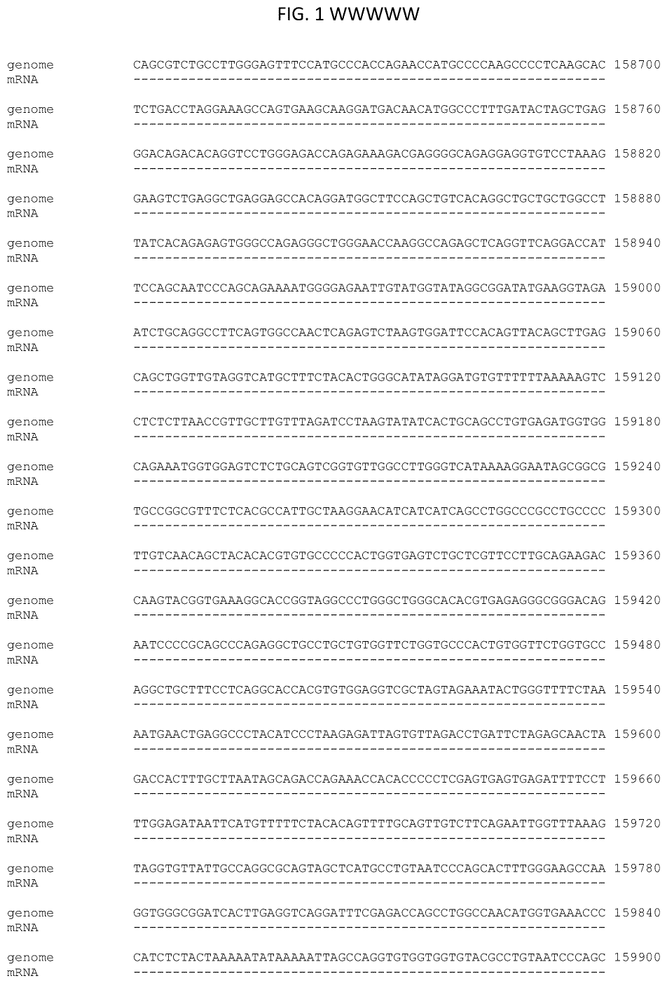

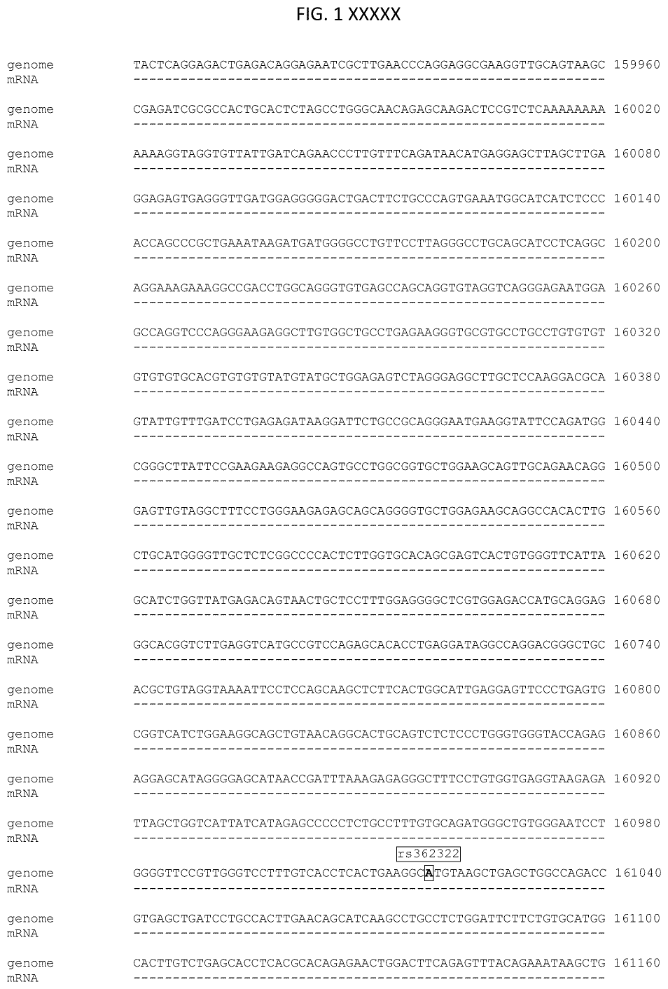

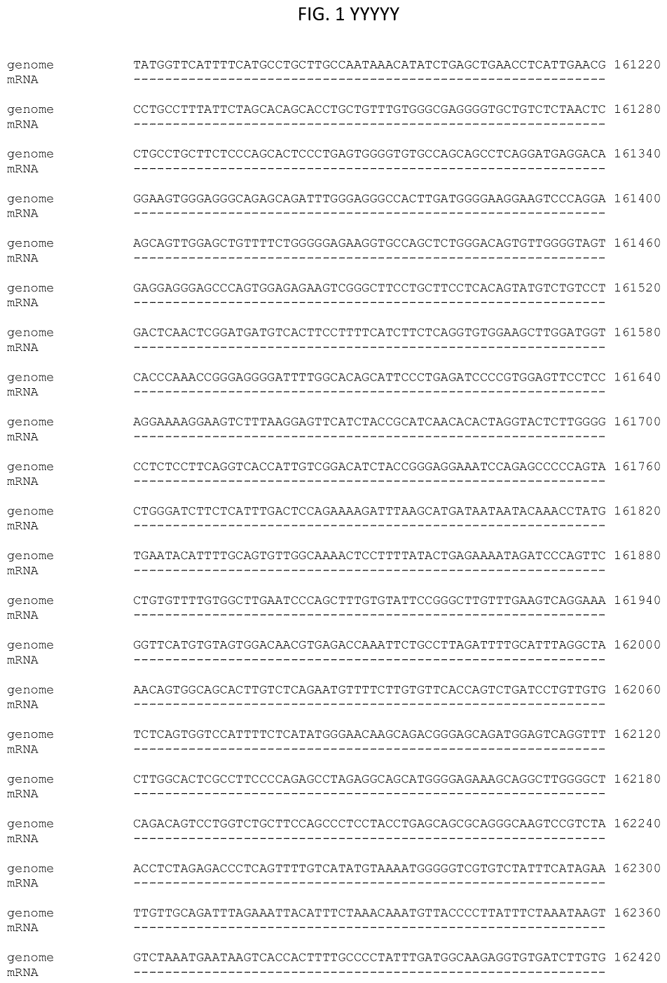

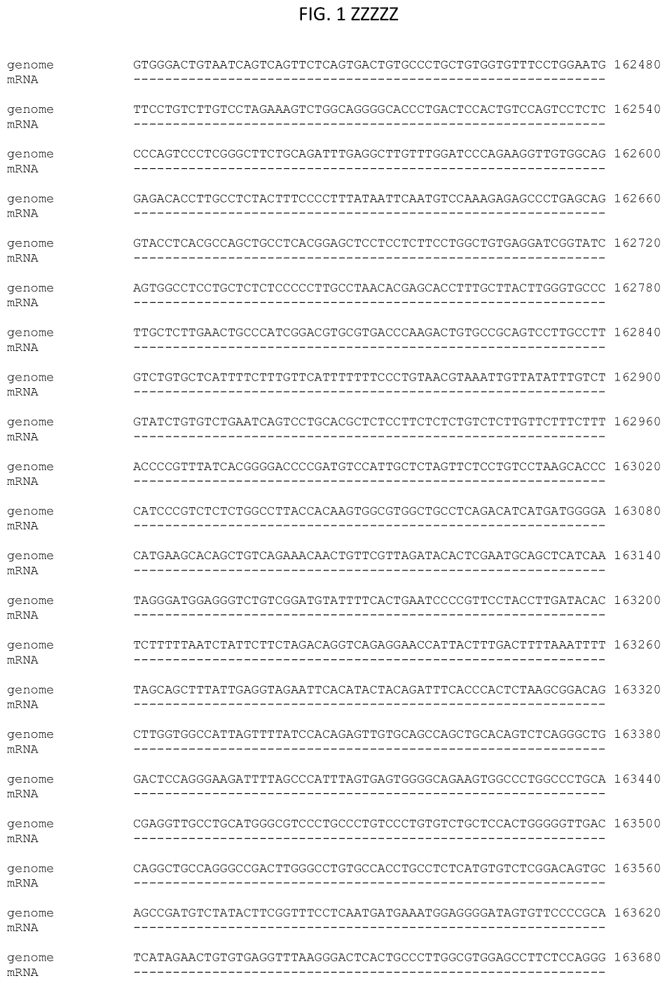

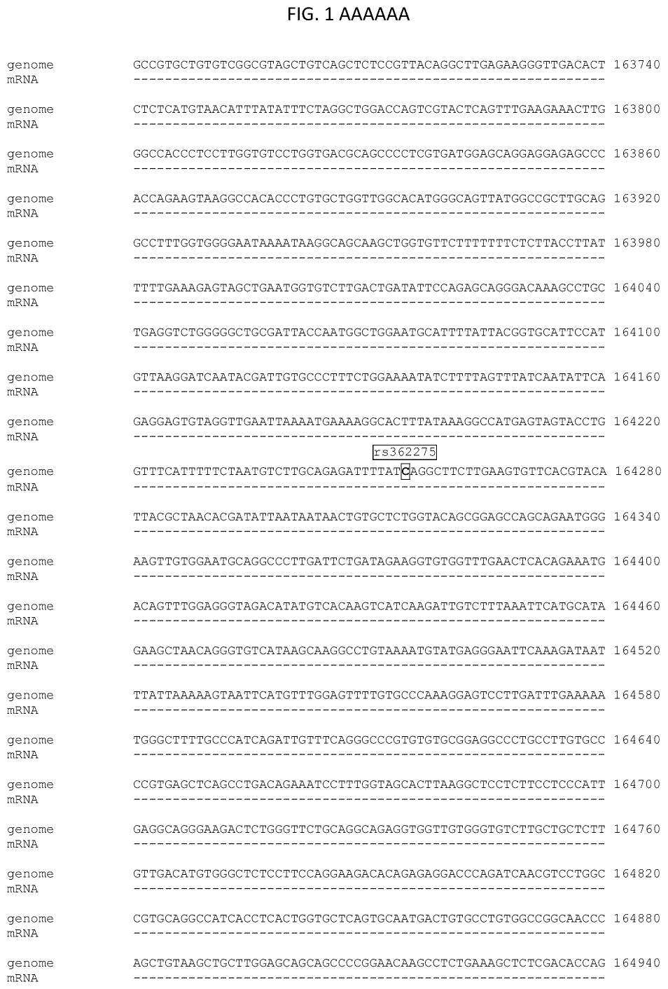

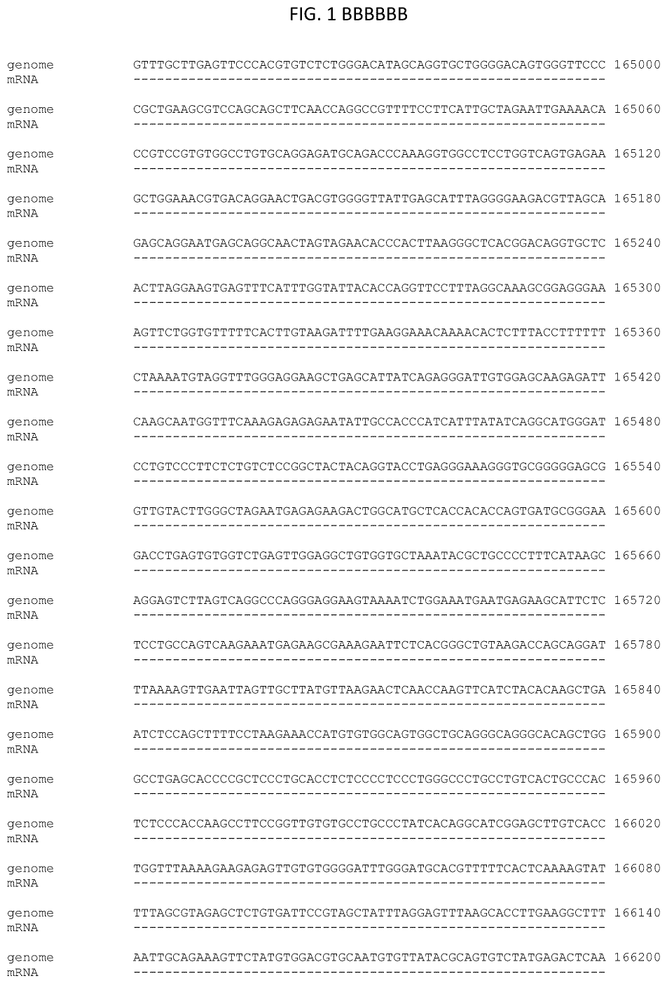

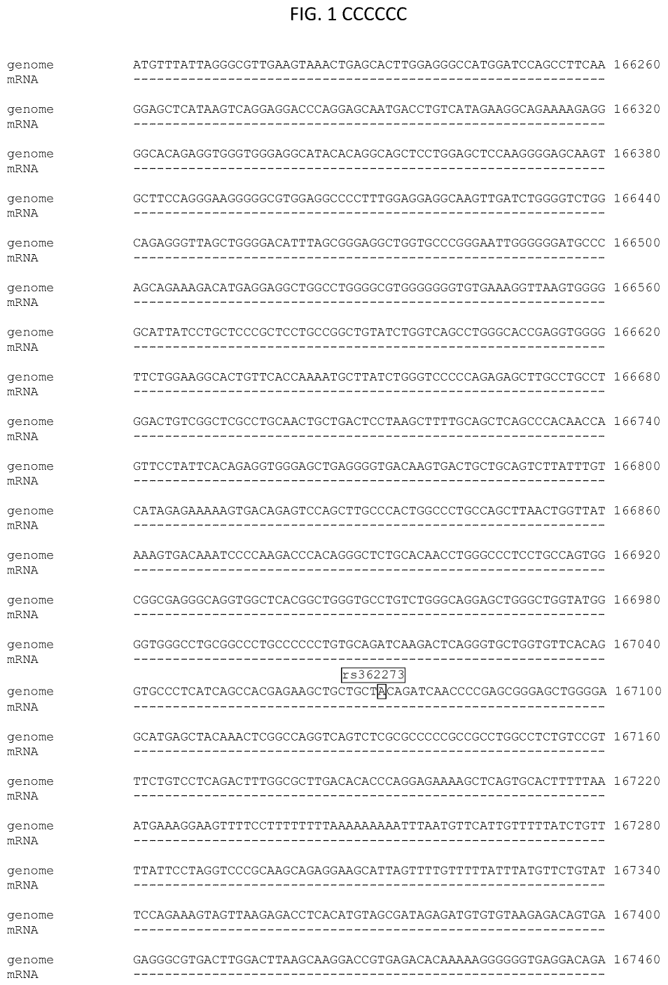

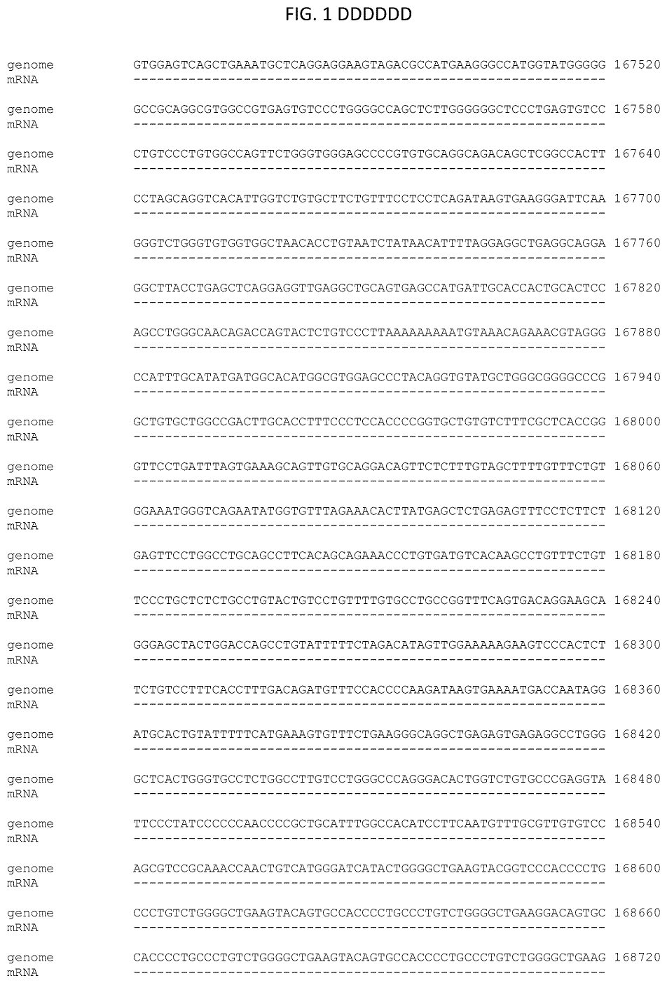

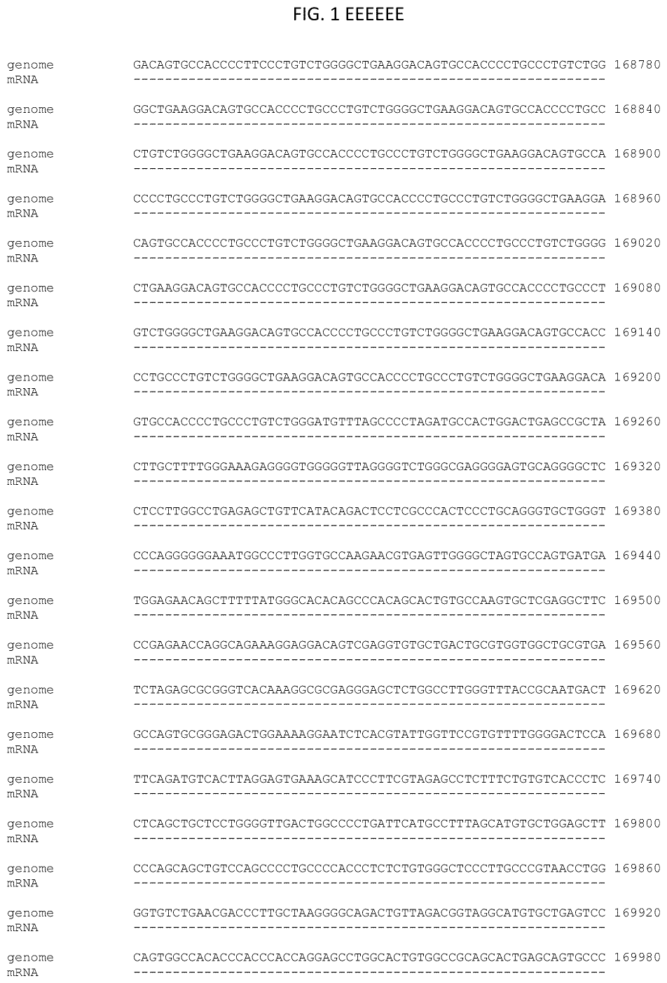

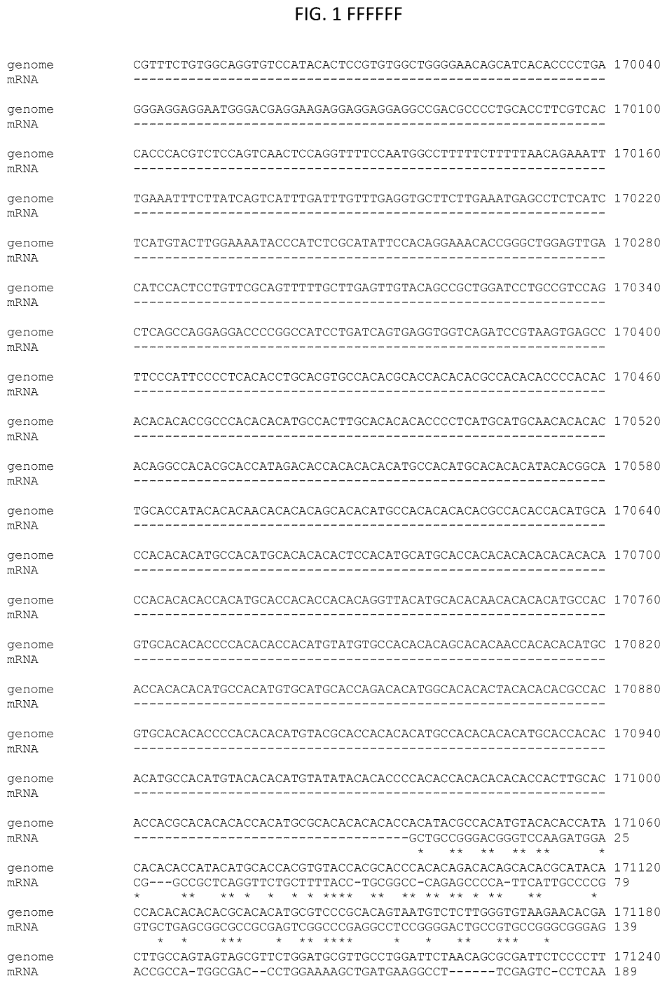

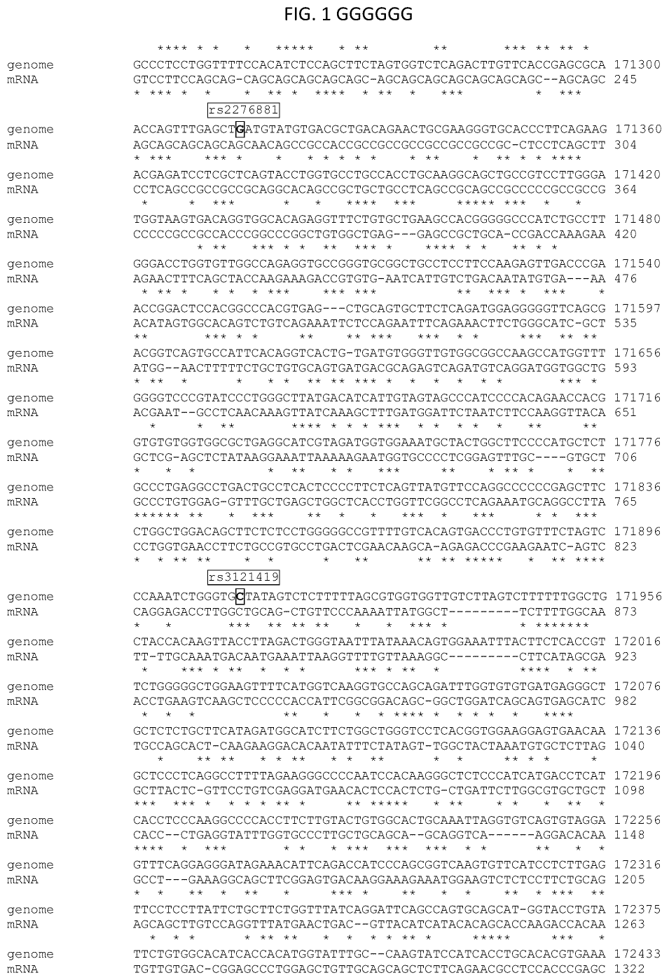

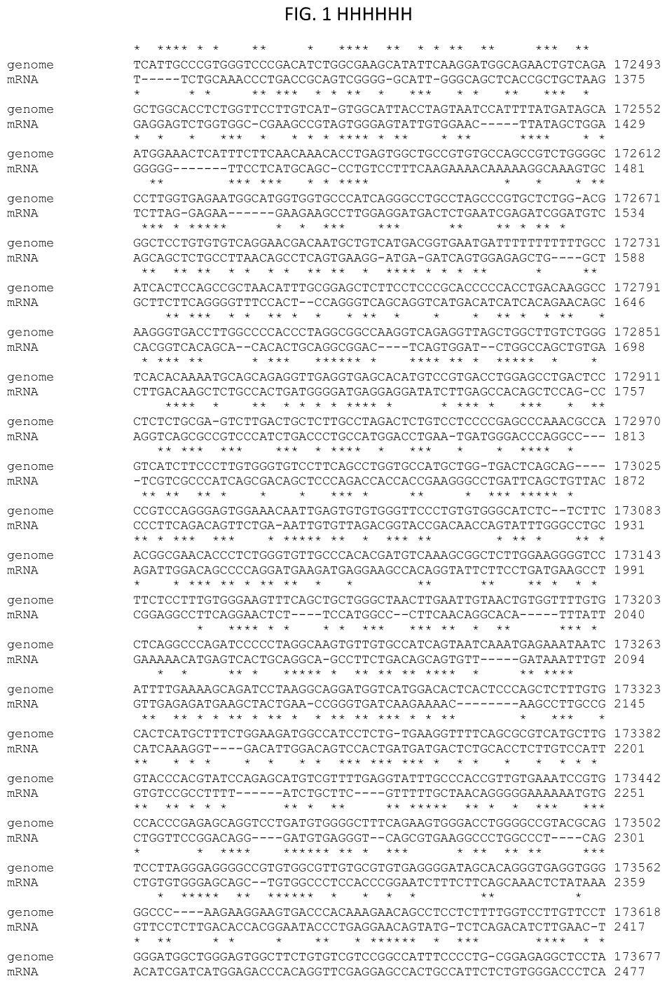

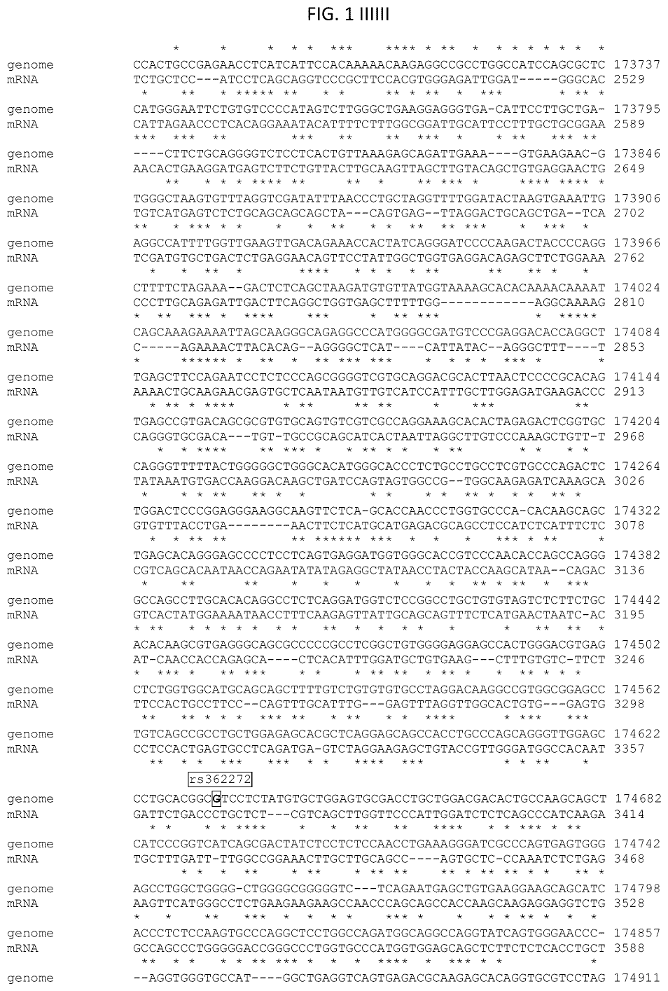

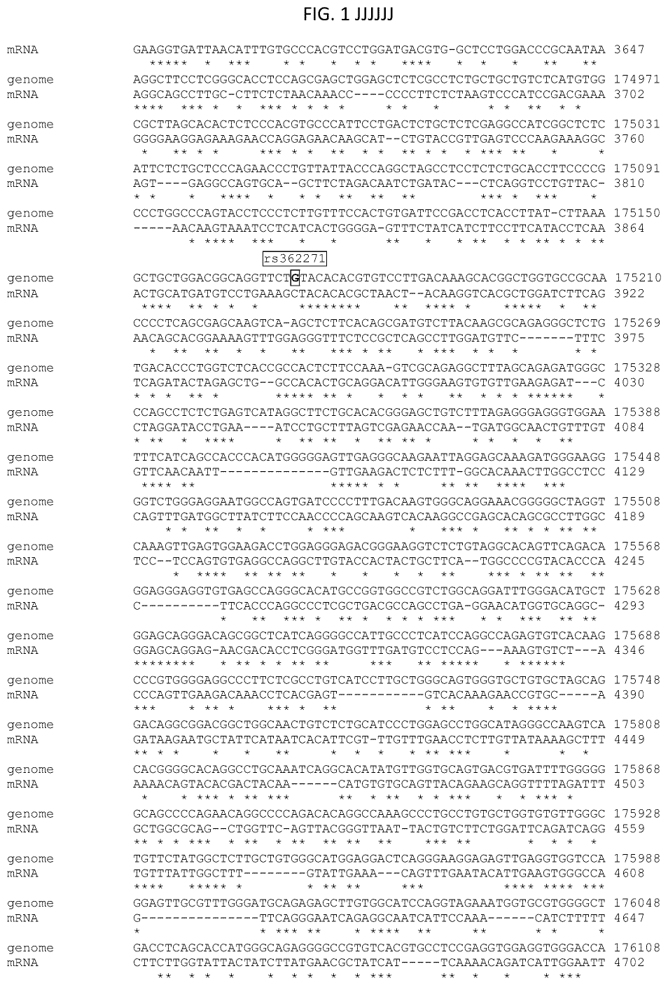

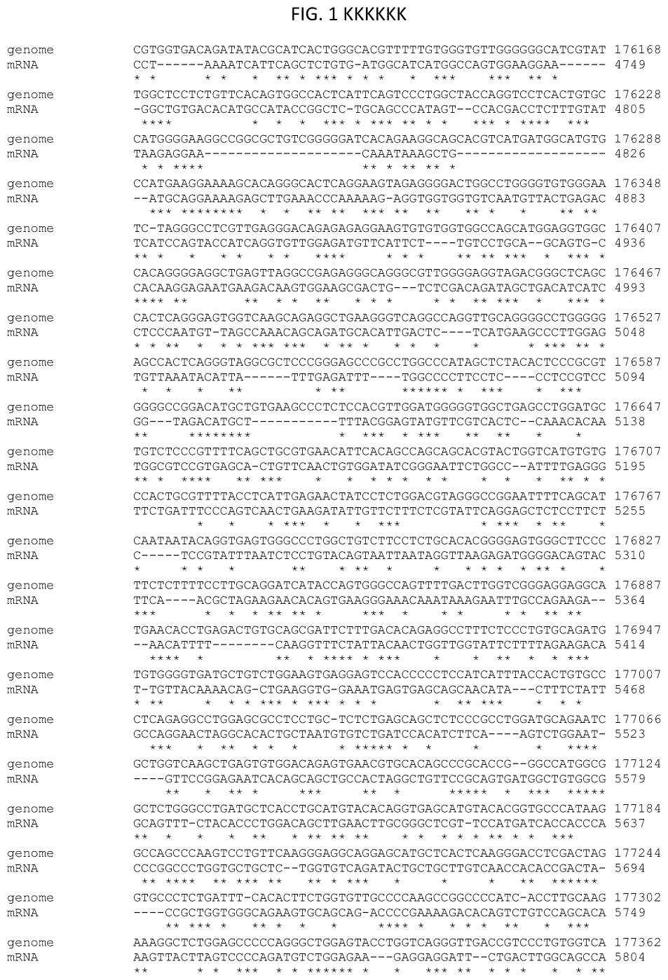

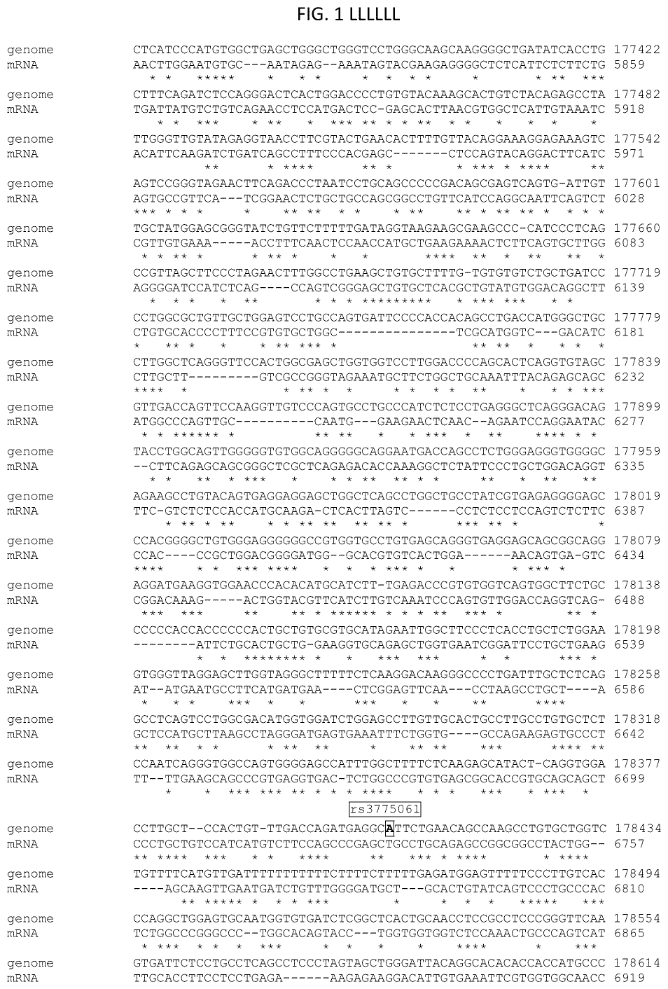

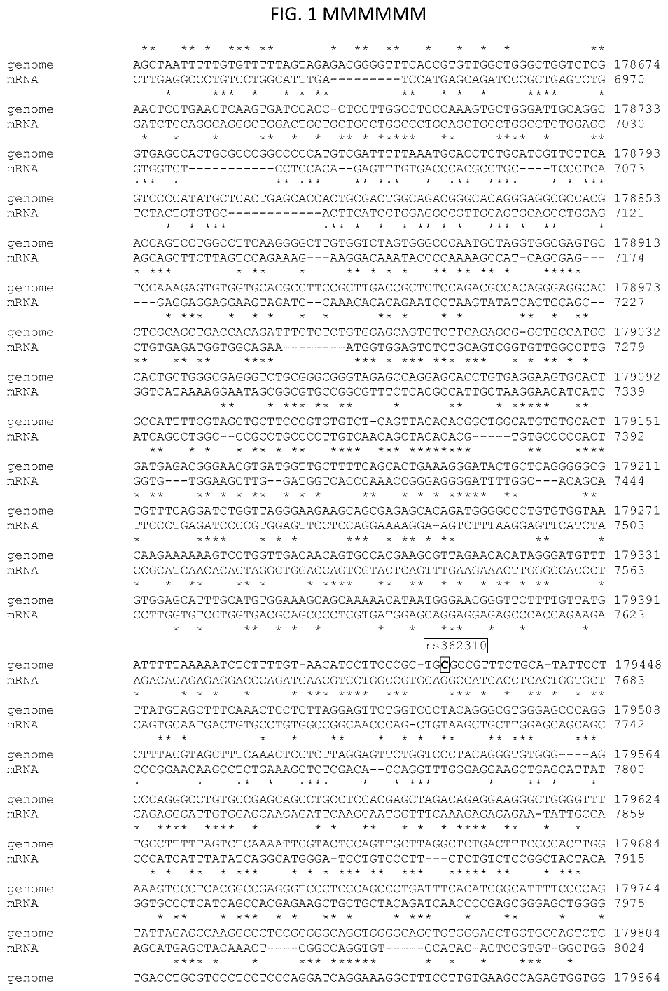

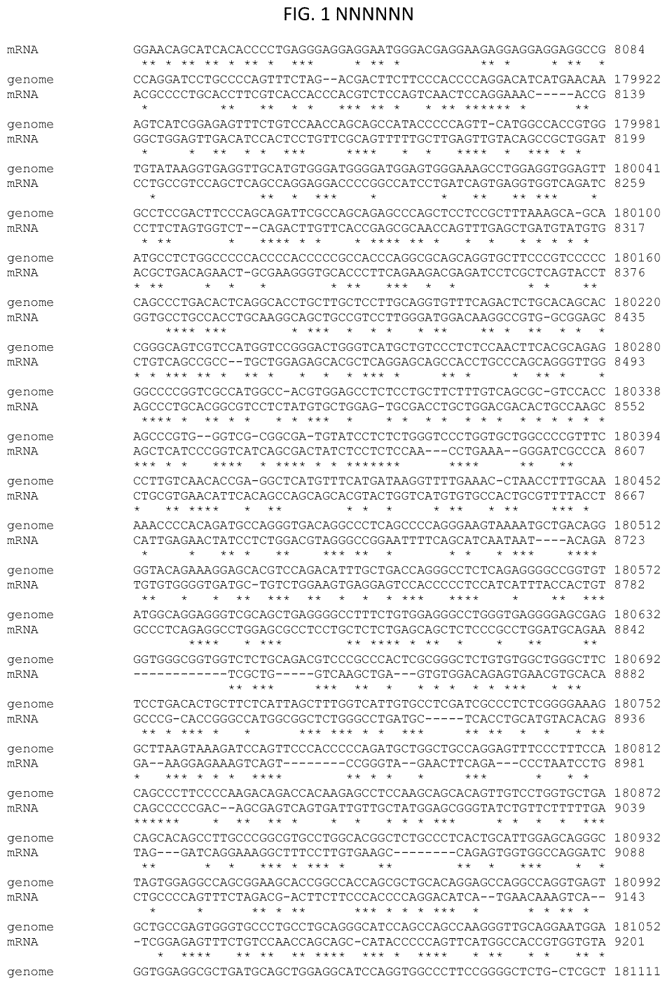

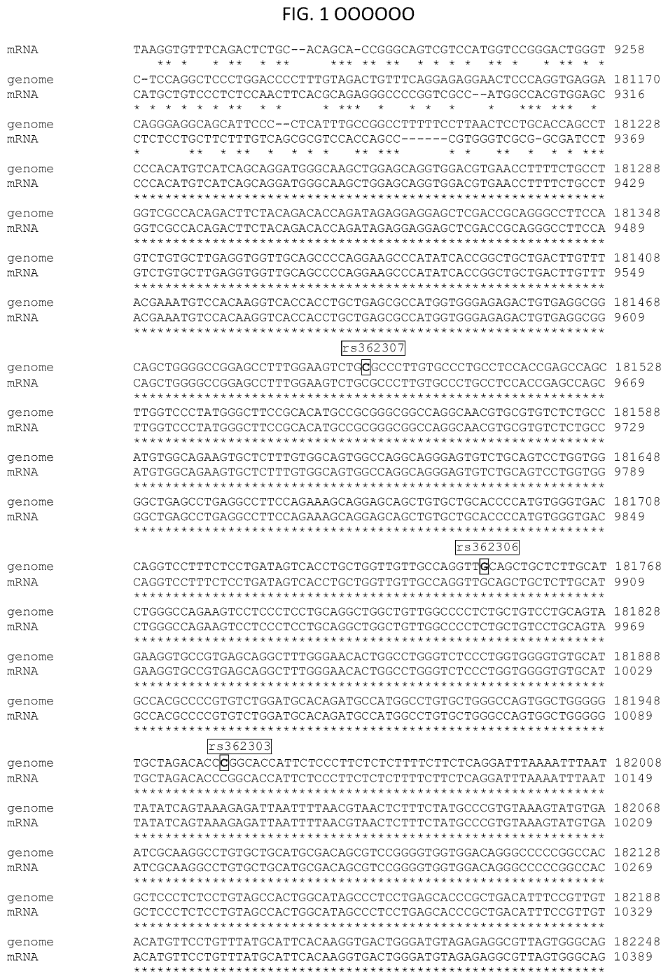

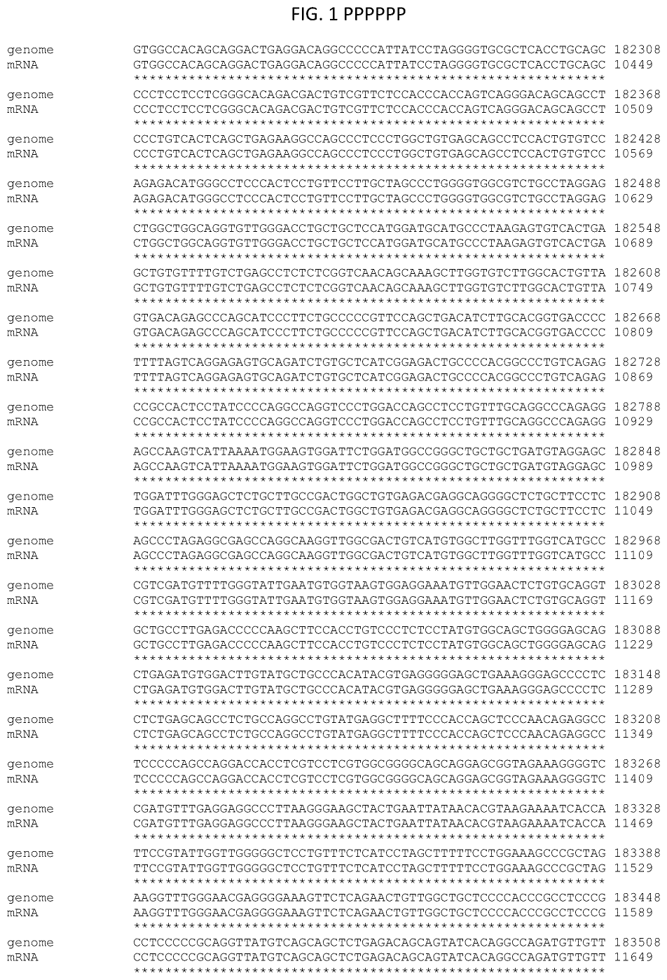

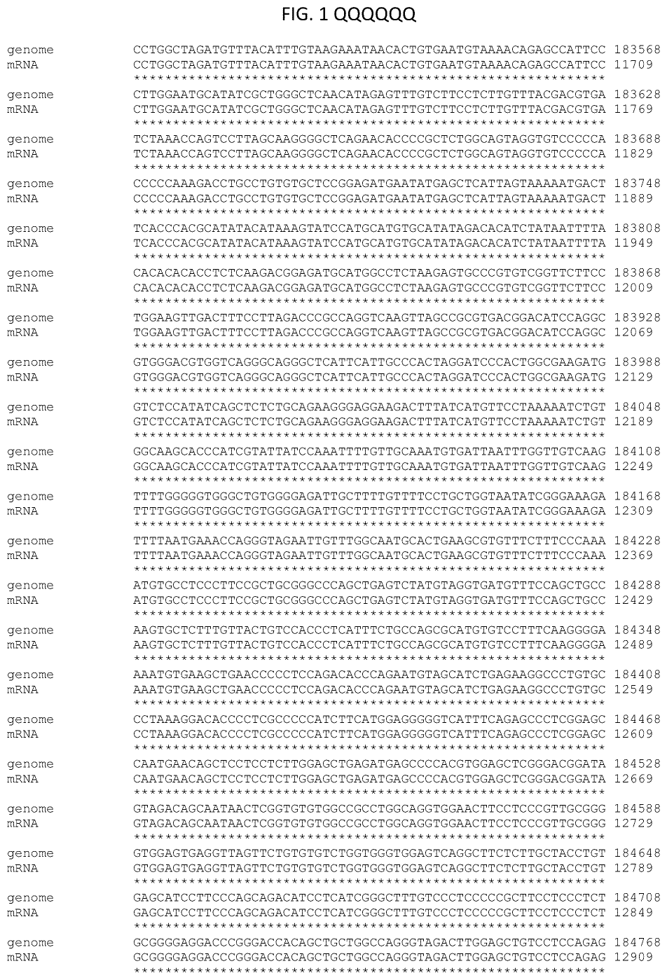

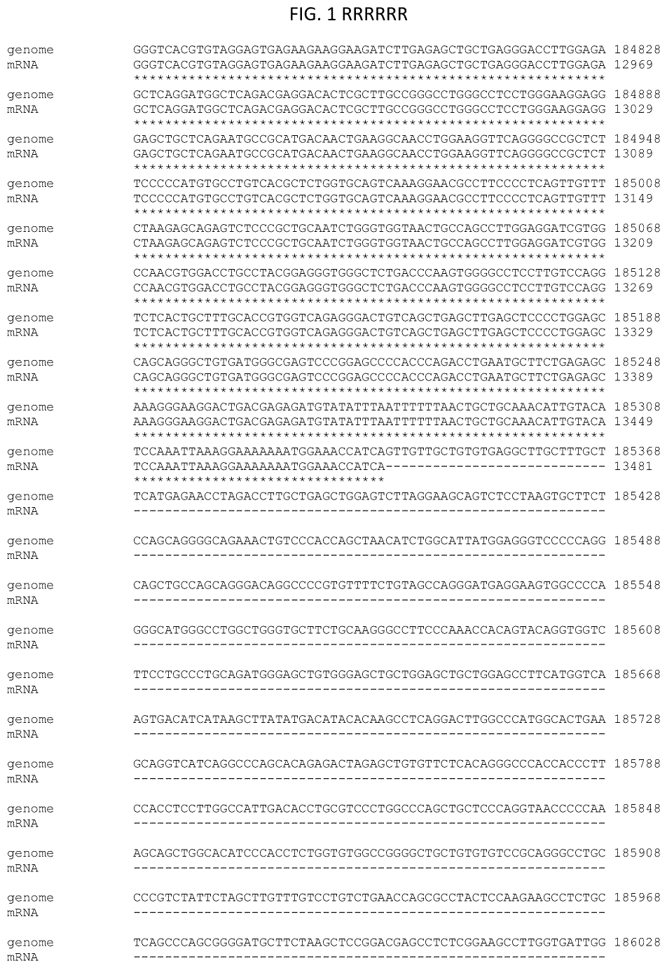

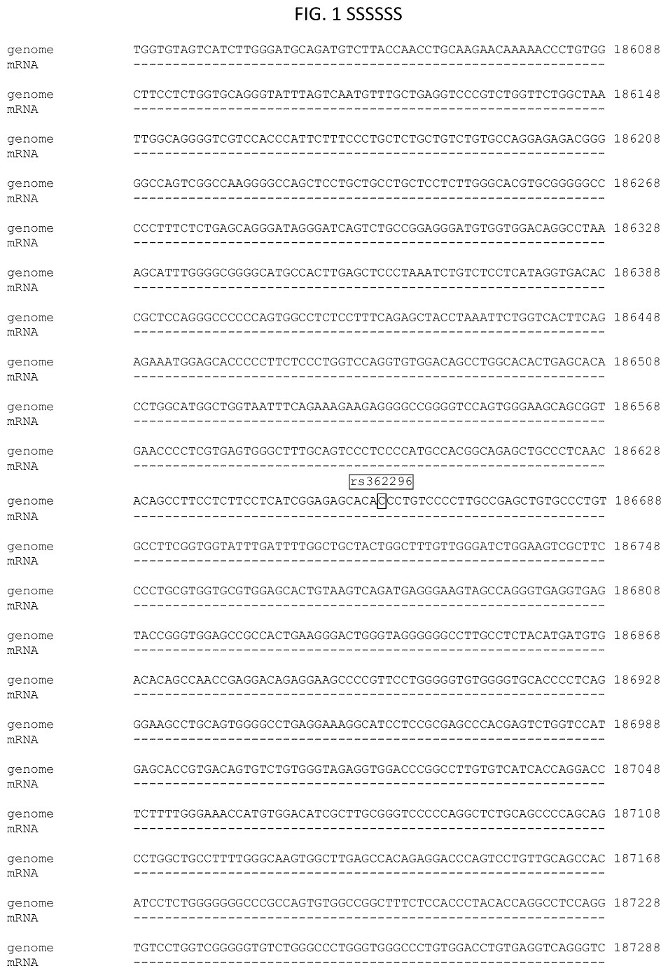

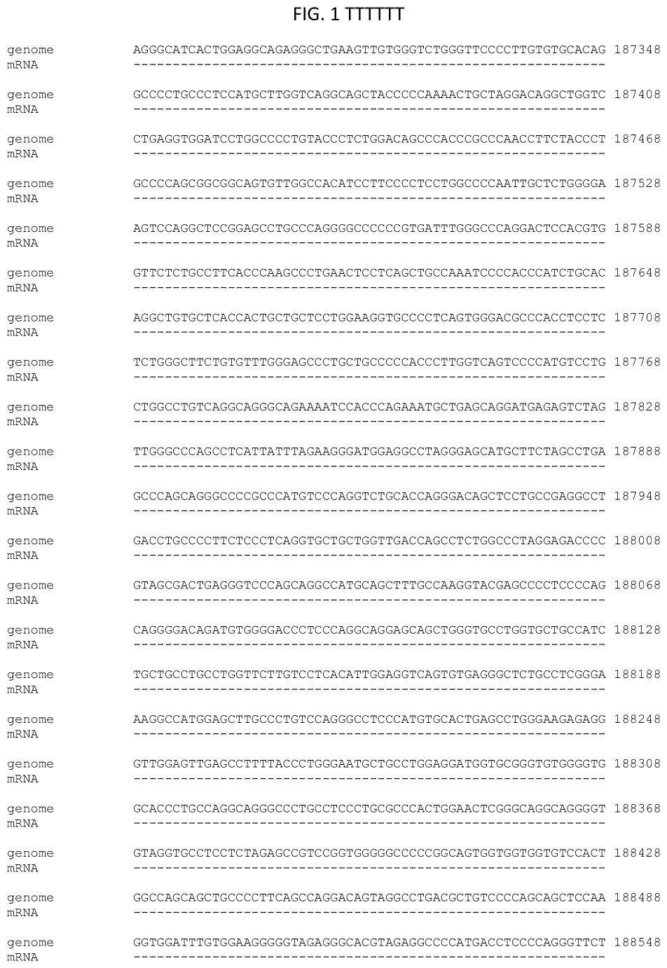

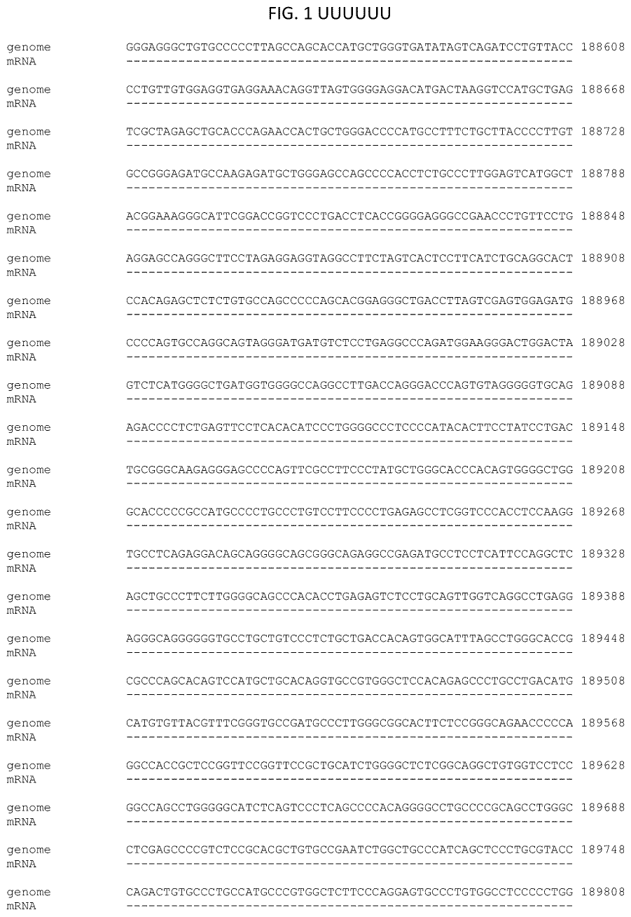

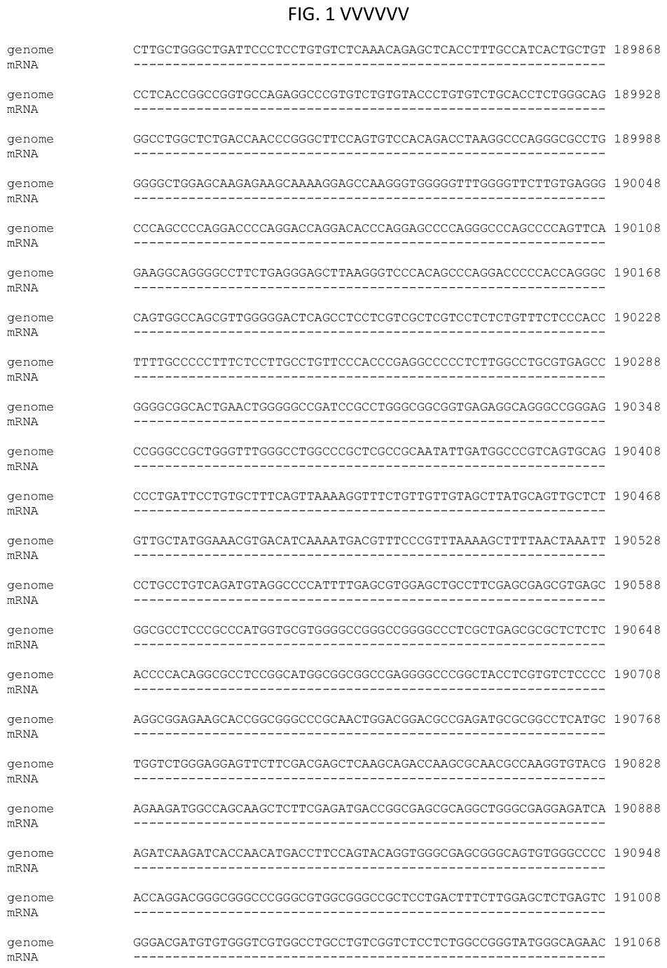

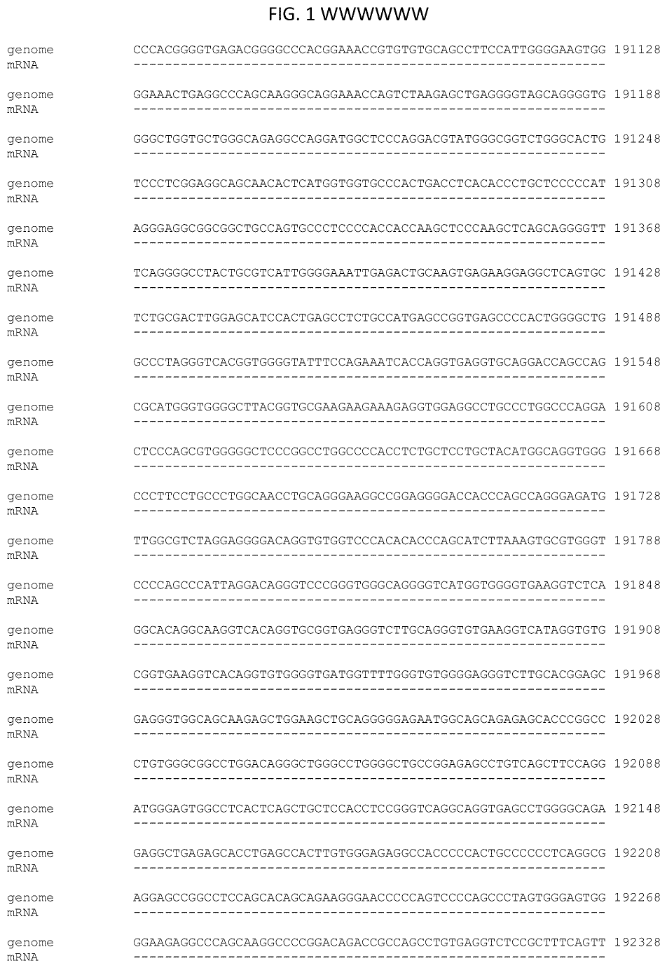

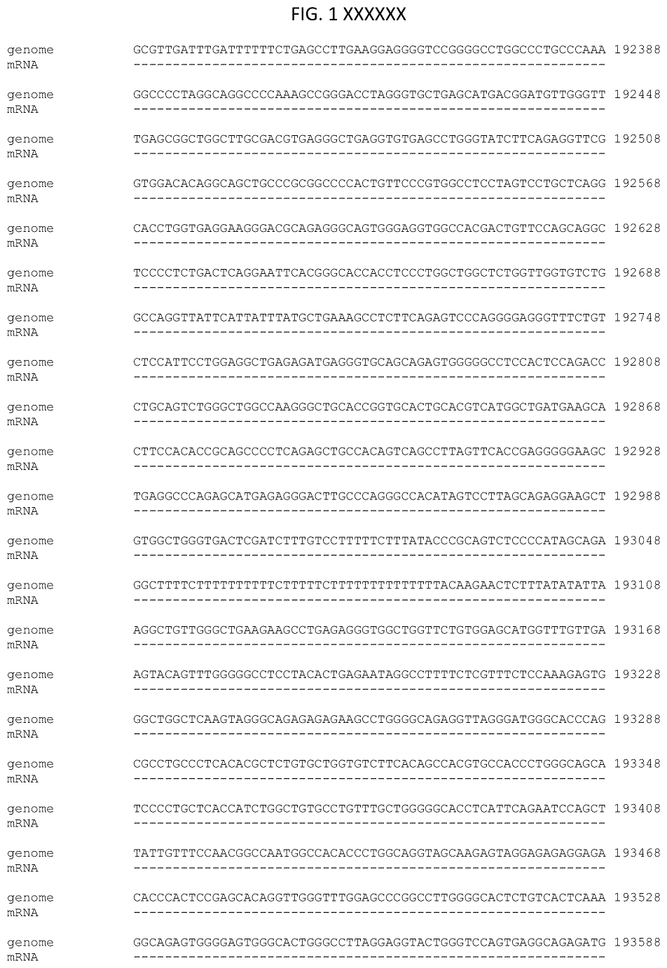

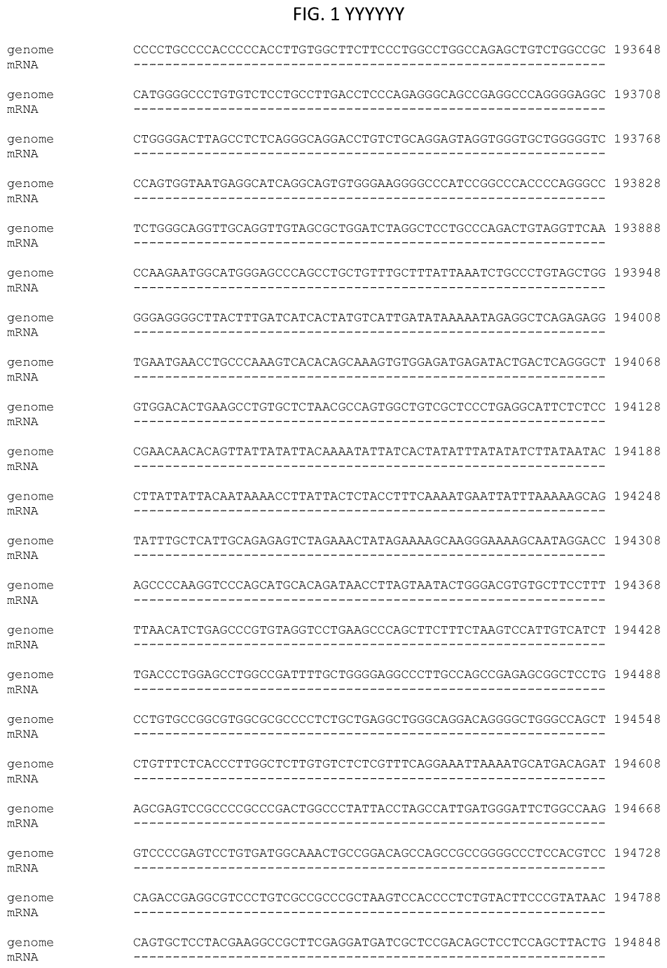

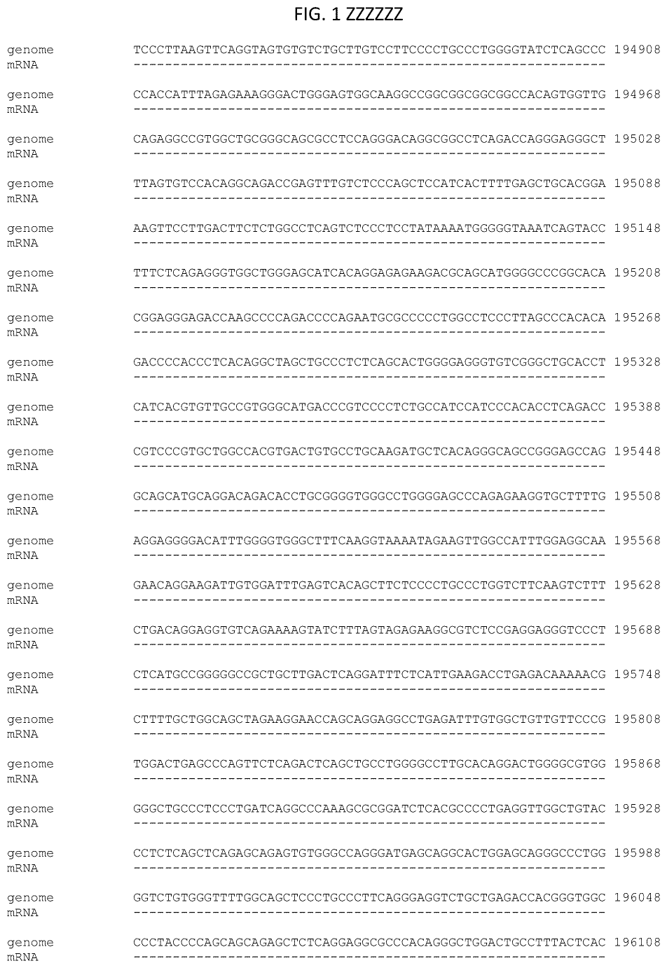

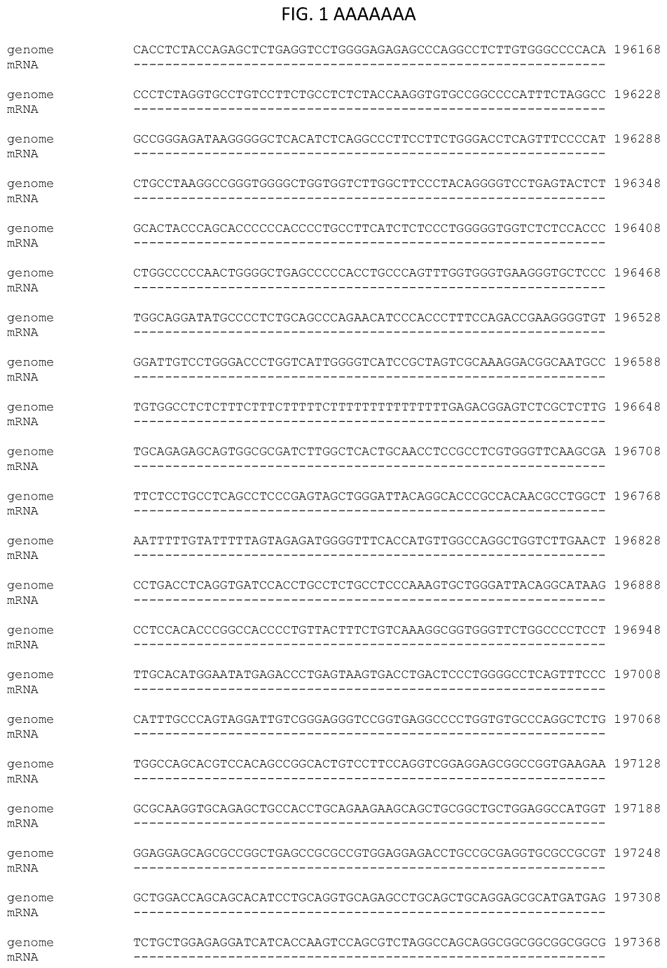

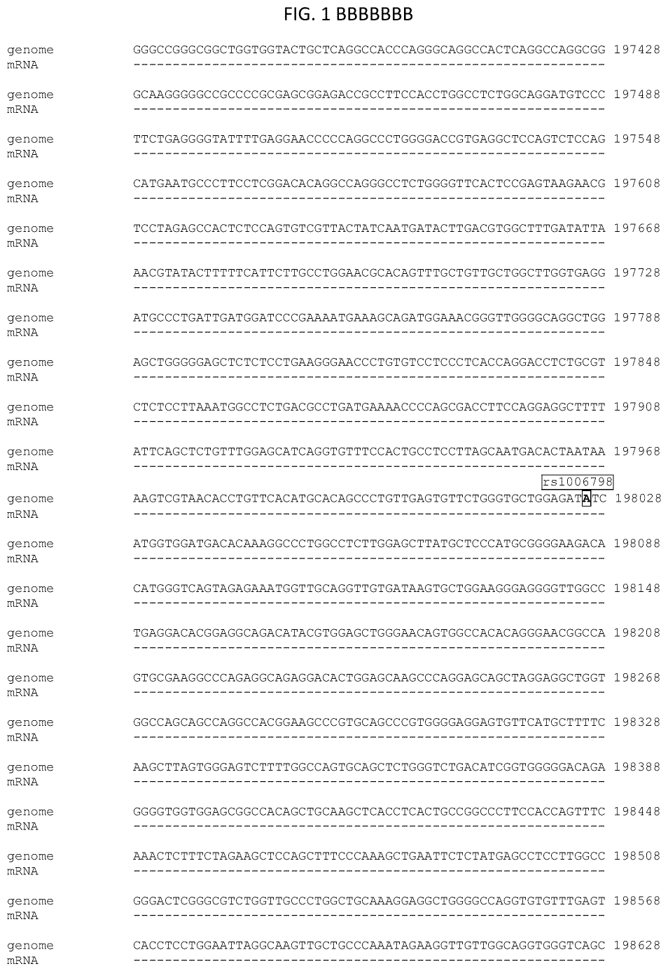

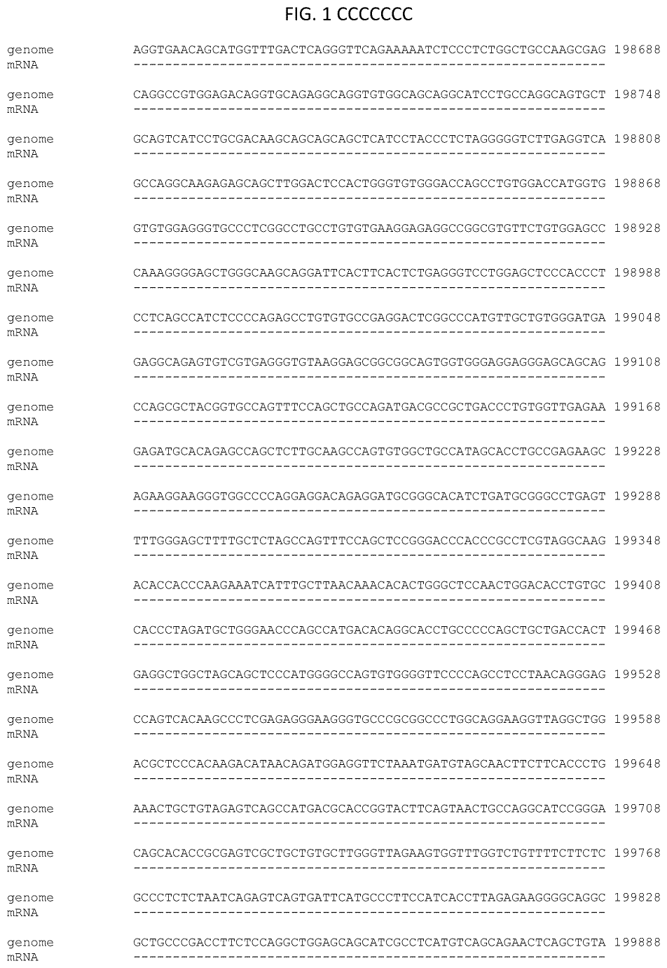

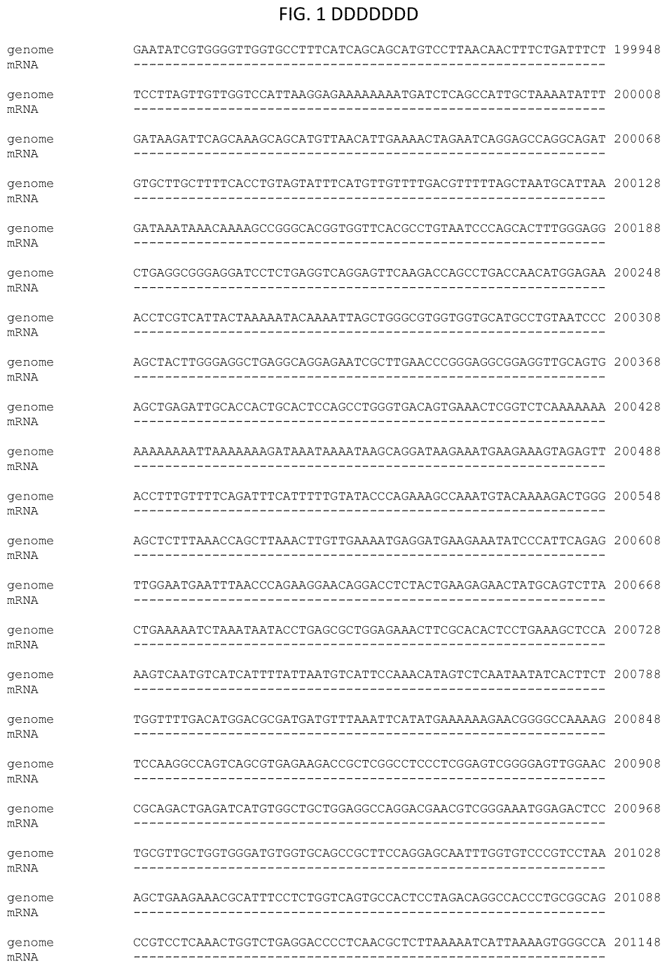

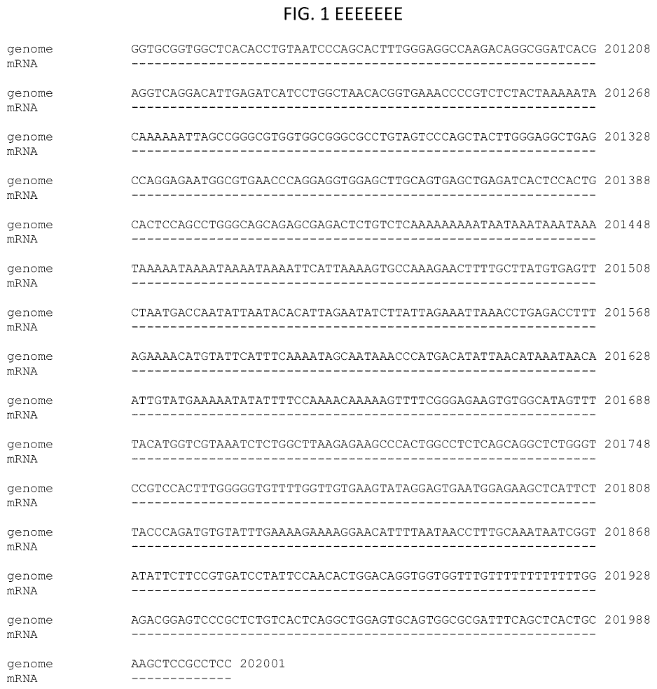

The HTT genomic sequence, designated herein as SEQ ID NO: 1 (NT_006081.18 truncated from nucleotides 1566000 to 1768000) was aligned with the HTT mRNA, designated herein as SEQ ID NO: 2 (NM_002111.6), using the EMBL-EBI sequence database (ClustalW2, http://www.ebi.ac.uk/Tools/clustalw2/index.html), and the output is presented in FIG. 1. SNP positions (identified by Hayden et al, WO/2009/135322) associated with the HTT gene were mapped to the two sequences and have been demarcated in FIG. 1 by their reference SNP ID number from the Entrez SNP database at the National Center for Biotechnology Information (NCBI, http://www.ncbi.nlm.nih.gov/sites/entrez?db=snp), incorporated herein by reference. Table 2 furnishes further details on each SNP. The ‘Reference SNP ID number’ or ‘RS number’ is the number designated to each SNP from the Entrez SNP database at NCBI, incorporated herein by reference. ‘SNP position’ refers to the nucleotide position of the SNP on SEQ ID NO: 1. ‘Polymorphism’ indicates the nucleotide variants at that SNP position. ‘Major allele’ indicates the nucleotide associated with the major allele, or the nucleotide present in a statistically significant proportion of individuals in the human population. ‘Minor allele’ indicates the nucleotide associated with the minor allele, or the nucleotide present in a relatively small proportion of individuals in the human population.

TABLE 2
Single Nuclear Polymorphisms (SNPs) and their positions on
SEQ ID NO: 1

| | SNP | | Major | Minor |
|---|---|---|---|---|
| RS No. | position | Polymorphism | allele | allele |
| rs2857936 | 1963 | C/T | C | T |
| rs12506200 | 3707 | A/G | G | A |
| rs762855 | 14449 | A/G | G | A |
| rs3856973 | 19826 | G/A | G | A |
| rs2285086 | 28912 | G/A | A | G |
| rs7659144 | 37974 | C/G | C | G |
| rs16843804 | 44043 | C/T | C | T |
| rs2024115 | 44221 | G/A | A | G |
| rs10015979 | 49095 | A/G | A | G |
| rs7691627 | 51063 | A/G | G | A |
| rs2798235 | 54485 | G/A | G | A |
| rs4690072 | 62160 | G/T | T | G |
| rs6446723 | 66466 | C/T | T | C |
| rs363081 | 73280 | G/A | G | A |
| rs363080 | 73564 | T/C | C | T |
| rs363075 | 77327 | G/A | G | A |
| rs363064 | 81063 | T/C | C | T |
| rs3025849 | 83420 | A/G | A | G |
| rs6855981 | 87929 | A/G | G | A |
| rs363102 | 88669 | G/A | A | G |
| rs11731237 | 91466 | C/T | C | T |
| rs4690073 | 99803 | A/G | G | A |
| rs363144 | 100948 | T/G | T | G |
| rs3025838 | 101099 | C/T | C | T |
| rs34315806 | 101687 | A/G | G | A |
| rs363099 | 101709 | T/C | C | T |
| rs363096 | 119674 | T/C | T | C |
| rs2298967 | 125400 | C/T | T | C |
| rs2298969 | 125897 | A/G | G | A |
| rs6844859 | 130139 | C/T | T | C |
| rs363092 | 135682 | C/A | C | A |
| rs7685686 | 146795 | A/G | A | G |
| rs363088 | 149983 | A/T | A | T |
| rs362331 | 155488 | C/T | T | C |
| rs916171 | 156468 | G/C | C | G |
| rs362322 | 161018 | A/G | A | G |
| rs362275 | 164255 | T/C | C | T |
| rs362273 | 167080 | A/G | A | G |
| rs2276881 | 171314 | G/A | G | A |
| rs3121419 | 171910 | T/C | C | T |
| rs362272 | 174633 | G/A | G | A |
| rs362271 | 175171 | G/A | G | A |
| rs3775061 | 178407 | C/T | C | T |
| rs362310 | 179429 | A/G | G | A |
| rs362307 | 181498 | T/C | C | T |
| rs362306 | 181753 | G/A | G | A |
| rs362303 | 181960 | T/C | C | T |
| rs362296 | 186660 | C/A | C | A |
| rs1006798 | 198026 | A/G | A | G |

### Example 2: Design of Antisense Oligonucleotides Targeting Huntingtin Gene SNPs and Inhibition of HTT mRNA in Coriell Fibroblast Cell Lines (GM04281, GM02171, and GM02173B)

Antisense oligonucleotides targeting nucleotides overlapping SNP positions presented in Table 1 were designed and tested for potency in three huntingtin patient-derived Coriell fibroblast cell lines, GM04281, GM02171, and GM02173B (from the Coriell Institute for Medical Research). Cultured GM04281 cells or GM02171 cells or GM02173B cells at a density of 20,000 cells per well were transfected using electroporation with 10,000 nM antisense oligonucleotide. After a treatment period of approximately 24 hours, RNA was isolated from the cells and HTT mRNA levels were measured by quantitative real time PCR using primer probe set RTS2617 (forward sequence CTCCGTCCGGTAGACATGCT, designated herein as SEQ ID NO: 3; reverse sequence GGAAATCAGAACCCTCAAAATGG, designated herein as SEQ ID NO: 4; probe sequence TGAGCACTGTTCAACTGTGGATATCGGGAX, designated herein as SEQ ID NO: 5). HTT mRNA levels were adjusted according to total RNA content, as measured by RIBOGREEN. Results are presented as percent inhibition of HTT mRNA, relative to untreated control cells.

ISIS 387916 (TCTCTATTGCACATTCCAAG, 5-10-5 MOE (SEQ ID NO: 6)) and ISIS 388816 (GCCGTAGCCTGGGACCCGCC, 5-10-5 MOE (SEQ ID NO: 7)) were included in each study as benchmark oligonucleotides against which the potency of the antisense oligonucleotides targeting nucleotides overlapping each SNP position could be compared.

The chimeric antisense oligonucleotides in Tables 3 and 4 were designed as 5-9-5 MOE gapmers. The gapmers are 19 nucleotides in length, wherein the central gap segment is comprised of nine 2′-deoxynucleotides and is flanked on both sides (in the 5′ and 3′ directions) by wings comprising five nucleotides each. Each nucleotide in the 5′ wing segment and each nucleotide in the 3′ wing segment has a 2′-MOE modification. The internucleoside linkages throughout each gapmer are phosphorothioate (P═S) linkages. All cytosine nucleobases throughout each gapmer are 5-methylcytosines.

The oligonucleotides are further described in Table 3. The percent inhibition of HTT mRNA by the antisense oligonucleotides in each cell line is shown in Table 4. ‘Target allele’ indicates whether the gapmer is targeted to the major or the minor allele at the SNP position. The number in parentheses indicates the nucleotide position in the gapmer opposite to the SNP position, starting from the 5′-terminus of the oligonucleotide. ‘Start site’ indicates the 5′-most nucleotide to which the gapmer is targeted. “Stop site” indicates the 3′-most nucleotide to which the gapmer is targeted. Each gapmer listed in Tables 3 and 4 is targeted to human HTT pre-mRNA, which is SEQ ID NO: 1.

TABLE 3
Chimeric oligonucleotides targeting SNP positions on the HTT gene

| | | | | | | SEQ |
|---|---|---|---|---|---|---|
| ISIS | SNP RS | Target | | Start | Stop | ID |
| No | No. | allele | Sequence | Site | Site | NO |
| 387916 | n/a | n/a | TCTCTATTGCACATTCCAAG | 145466 | 145485 | 6 |
| 388816 | n/a | n/a | GCCGTAGCCTGGGACCCGCC | 16501 | 16520 | 7 |
| 435330 | rs3856973 | Major (8) | TAACACTCGATTAACCCTG | 19815 | 19833 | 8 |
| 435348 | rs3856973 | Minor (8) | TAACACTTGATTAACCCTG | 19815 | 19833 | 9 |
| 435294 | rs3856973 | Major (10) | GTTAACACTCGATTAACCC | 19817 | 19835 | 10 |
| 435312 | rs3856973 | Minor (10) | GTTAACACTTGATTAACCC | 19817 | 19835 | 11 |
| 435864 | rs2285086 | Major (10) | GCTAGTTCATCCCAGTGAG | 28903 | 28921 | 12 |
| 435889 | rs2285086 | Minor (10) | GCTAGTTCACCCCAGTGAG | 28903 | 28921 | 13 |
| 435878 | rs7659144 | Major (10) | TGGAAATGGGTTTTTCCAC | 37965 | 37983 | 14 |
| 435903 | rs7659144 | Minor (10) | TGGAAATGGCTTTTTCCAC | 37965 | 37983 | 15 |
| 435863 | rs16843804 | Major (10) | TTTAACCGTGGCATGGGCA | 44034 | 44052 | 16 |
| 435888 | rs16843804 | Minor (10) | TTTAACCGTAGCATGGGCA | 44034 | 44052 | 17 |
| 435331 | rs2024115 | Major (8) | TTCAAGCTAGTAACGATGC | 44210 | 44228 | 18 |
| 435349 | rs2024115 | Minor (8) | TTCAAGCCAGTAACGATGC | 44210 | 44228 | 19 |
| 435295 | rs2024115 | Major (10) | ACTTCAAGCTAGTAACGAT | 44212 | 44230 | 20 |
| 435313 | rs2024115 | Minor (10) | ACTTCAAGCCAGTAACGAT | 44212 | 44230 | 21 |
| 435862 | rs10015979 | Major (10) | GCAGCTAGGTTAAAGAGTC | 49086 | 49104 | 22 |
| 435887 | rs10015979 | Minor (10) | GCAGCTAGGCTAAAGAGTC | 49086 | 49104 | 23 |
| 435880 | rs7691627 | Major (10) | AATAAGAAACACAATCAAA | 51054 | 51072 | 24 |
| 435905 | rs7691627 | Minor (10) | AATAAGAAATACAATCAAA | 51054 | 51072 | 25 |
| 435885 | rs2798235 | Major (10) | CAGAGGAGGCATACTGTAT | 54476 | 54494 | 26 |
| 435910 | rs2798235 | Minor (10) | CAGAGGAGGTATACTGTAT | 54476 | 54494 | 27 |
| 435874 | rs4690072 | Major (10) | CACAGTGCTACCCAACCTT | 62151 | 62169 | 28 |
| 435899 | rs4690072 | Minor (10) | CACAGTGCTCCCCAACCTT | 62151 | 62169 | 29 |
| 435875 | rs6446723 | Major (10) | TAATTTTCTAGACTTTATG | 66457 | 66475 | 30 |
| 435900 | rs6446723 | Minor (10) | TAATTTTCTGGACTTTATG | 66457 | 66475 | 31 |
| 435332 | rs363081 | Major (8) | GCTACAACGCAGGTCAAAT | 73269 | 73287 | 32 |
| 435350 | rs363081 | Minor (8) | GCTACAATGCAGGTCAAAT | 73269 | 73287 | 33 |
| 435296 | rs363081 | Major (10) | GAGCTACAACGCAGGTCAA | 73271 | 73289 | 34 |
| 435314 | rs363081 | Minor (10) | GAGCTACAATGCAGGTCAA | 73271 | 73289 | 35 |
| 435886 | rs363080 | Major (10) | AGAGAGAACGAGAAGGCTC | 73555 | 73573 | 36 |
| 435911 | rs363080 | Minor (10) | AGAGAGAACAAGAAGGCTC | 73555 | 73573 | 37 |
| 435914 | rs363075 | Major (6) | AGCCCCTCTGTGTAAGTTT | 77314 | 77332 | 38 |
| 435926 | rs363075 | Minor (6) | AGCCCTTCTGTGTAAGTTT | 77314 | 77332 | 39 |
| 435916 | rs363075 | Major (7) | GAGCCCCTCTGTGTAAGTT | 77315 | 77333 | 40 |
| 435928 | rs363075 | Minor (7) | GAGCCCTTCTGTGTAAGTT | 77315 | 77333 | 41 |
| 435333 | rs363075 | Major (8) | TGAGCCCCTCTGTGTAAGT | 77316 | 77334 | 42 |
| 435351 | rs363075 | Minor (8) | TGAGCCCTTCTGTGTAAGT | 77316 | 77334 | 43 |
| 435918 | rs363075 | Major (9) | ATGAGCCCCTCTGTGTAAG | 77317 | 77335 | 44 |
| 435930 | rs363075 | Minor (9) | ATGAGCCCTTCTGTGTAAG | 77317 | 77335 | 45 |
| 435297 | rs363075 | Major (10) | GATGAGCCCCTCTGTGTAA | 77318 | 77336 | 46 |
| 435315 | rs363075 | Minor (10) | GATGAGCCCTTCTGTGTAA | 77318 | 77336 | 47 |
| 435920 | rs363075 | Major (11) | TGATGAGCCCCTCTGTGTA | 77319 | 77337 | 48 |
| 435932 | rs363075 | Minor (11) | TGATGAGCCCTTCTGTGTA | 77319 | 77337 | 49 |
| 435366 | rs363075 | Major (12) | ATGATGAGCCCCTCTGTGT | 77320 | 77338 | 50 |
| 435924 | rs363075 | Minor (12) | ATGATGAGCCCTTCTGTGT | 77320 | 77338 | 51 |
| 435922 | rs363075 | Major (14) | TAATGATGAGCCCCTCTGT | 77322 | 77340 | 52 |
| 435934 | rs363075 | Minor (14) | TAATGATGAGCCCTTCTGT | 77322 | 77340 | 53 |
| 435334 | rs363064 | Major (8) | AGAATACGGGTAACATTTT | 81052 | 81070 | 54 |
| 435352 | rs363064 | Minor (8) | AGAATACAGGTAACATTTT | 81052 | 81070 | 55 |
| 435298 | rs363064 | Major (10) | GGAGAATACGGGTAACATT | 81054 | 81072 | 56 |
| 435316 | rs363064 | Minor (10) | GGAGAATACAGGTAACATT | 81054 | 81072 | 57 |
| 435335 | rs3025849 | Major (8) | TTAGTAATCAATTTTAATG | 83409 | 83427 | 58 |
| 435353 | rs3025849 | Minor (8) | TTAGTAACCAATTTTAATG | 83409 | 83427 | 59 |
| 435299 | rs3025849 | Major (10) | AGTTAGTAATCAATTTTAA | 83411 | 83429 | 60 |
| 435317 | rs3025849 | Minor (10) | AGTTAGTAACCAATTTTAA | 83411 | 83429 | 61 |
| 435877 | rs6855981 | Major (10) | GAAGGAATGCTTTTACTAG | 87920 | 87938 | 62 |
| 435902 | rs6855981 | Minor (10) | GAAGGAATGTTTTTACTAG | 87920 | 87938 | 63 |
| 435336 | rs363102 | Major (8) | CTAAAACTAACTTGAGAAT | 88658 | 88676 | 64 |
| 435354 | rs363102 | Minor (8) | CTAAAACCAACTTGAGAAT | 88658 | 88676 | 65 |
| 435300 | rs363102 | Major (10) | ATCTAAAACTAACTTGAGA | 88660 | 88678 | 66 |
| 435318 | rs363102 | Minor (10) | ATCTAAAACCAACTTGAGA | 88660 | 88678 | 67 |
| 435884 | rs11731237 | Major (10) | GGTGGGCAGGAAGGACTGA | 91457 | 91475 | 68 |
| 435909 | rs11731237 | Minor (10) | GGTGGGCAGAAAGGACTGA | 91457 | 91475 | 69 |
| 435337 | rs4690073 | Major (8) | CCTAAATCAATCTACAAGT | 99792 | 99810 | 70 |
| 435355 | rs4690073 | Minor (8) | CCTAAATTAATCTACAAGT | 99792 | 99810 | 71 |
| 435301 | rs4690073 | Major (10) | TCCCTAAATCAATCTACAA | 99794 | 99812 | 72 |
| 435319 | rs4690073 | Minor (10) | TCCCTAAATTAATCTACAA | 99794 | 99812 | 73 |
| 435883 | rs363144 | Major (10) | GAAAATGTGAGTGGATCTA | 100939 | 100957 | 74 |
| 435908 | rs363144 | Minor (10) | GAAAATGTGCGTGGATCTA | 100939 | 100957 | 75 |
| 435338 | rs3025838 | Major (8) | GTAAGGCGAGACTGACTAG | 101088 | 101106 | 76 |
| 435356 | rs3025838 | Minor (8) | GTAAGGCAAGACTGACTAG | 101088 | 101106 | 77 |
| 435302 | rs3025838 | Major (10) | AGGTAAGGCGAGACTGACT | 101090 | 101108 | 78 |
| 435320 | rs3025838 | Minor (10) | AGGTAAGGCAAGACTGACT | 101090 | 101108 | 79 |
| 435339 | rs363099 | Major (8) | CTGAGCGGAGAAACCCTCC | 101698 | 101716 | 80 |
| 435357 | rs363099 | Minor (8) | CTGAGCGAAGAAACCCTCC | 101698 | 101716 | 81 |
| 435303 | rs363099 | Major (10) | GGCTGAGCGGAGAAACCCT | 101700 | 101718 | 82 |
| 435321 | rs363099 | Minor (10) | GGCTGAGCGAAGAAACCCT | 101700 | 101718 | 83 |
| 435367 | rs363099 | Major (12) | AAGGCTGAGCGGAGAAACC | 101702 | 101720 | 84 |
| 435340 | rs363096 | Major (8) | TTCCCTAAAAACAAAAACA | 119663 | 119681 | 85 |
| 435358 | rs363096 | Minor (8) | TTCCCTAGAAACAAAAACA | 119663 | 119681 | 86 |
| 435304 | rs363096 | Major (10) | GATTCCCTAAAAACAAAAA | 119665 | 119683 | 87 |
| 435322 | rs363096 | Minor (10) | GATTCCCTAGAAACAAAAA | 119665 | 119683 | 88 |
| 435341 | rs2298967 | Major (8) | CTTTTCTATTGTCTGTCCC | 125389 | 125407 | 89 |
| 435359 | rs2298967 | Minor (8) | CTTTTCTGTTGTCTGTCCC | 125389 | 125407 | 90 |
| 435305 | rs2298967 | Major (10) | TGCTTTTCTATTGTCTGTC | 125391 | 125409 | 91 |
| 435323 | rs2298967 | Minor (10) | TGCTTTTCTGTTGTCTGTC | 125391 | 125409 | 92 |
| 435865 | rs2298969 | Major (10) | AAGGGATGCCGACTTGGGC | 125888 | 125906 | 93 |
| 435890 | rs2298969 | Minor (10) | AAGGGATGCTGACTTGGGC | 125888 | 125906 | 94 |
| 435876 | rs6844859 | Major (10) | ACCTTCCTCACTGAGGATG | 130130 | 130148 | 95 |
| 435901 | rs6844859 | Minor (10) | ACCTTCCTCGCTGAGGATG | 130130 | 130148 | 96 |
| 435872 | rs363092 | Major (10) | CAAACCACTGTGGGATGAA | 135673 | 135691 | 97 |
| 435897 | rs363092 | Minor (10) | CAAACCACTTTGGGATGAA | 135673 | 135691 | 98 |
| 435879 | rs7685686 | Major (10) | AATAAATTGTCATCACCAG | 146786 | 146804 | 99 |
| 435904 | rs7685686 | Minor (10) | AATAAATTGCCATCACCAG | 146786 | 146804 | 100 |
| 435871 | rs363088 | Major (10) | TCACAGCTATCTTCTCATC | 149974 | 149992 | 101 |
| 435896 | rs363088 | Minor (10) | TCACAGCTAACTTCTCATC | 149974 | 149992 | 102 |
| 435870 | rs362331 | Major (10) | GCACACAGTAGATGAGGGA | 155479 | 155497 | 103 |
| 435895 | rs362331 | Minor (10) | GCACACAGTGGATGAGGGA | 155479 | 155497 | 104 |
| 435881 | rs916171 | Major (10) | CAGAACAAAGAGAAGAATT | 156459 | 156477 | 105 |
| 435906 | rs916171 | Minor (10) | CAGAACAAACAGAAGAATT | 156459 | 156477 | 106 |
| 435342 | rs362322 | Major (8) | GCTTACATGCCTTCAGTGA | 161007 | 161025 | 107 |
| 435360 | rs362322 | Minor (8) | GCTTACACGCCTTCAGTGA | 161007 | 161025 | 108 |
| 435306 | rs362322 | Major (10) | CAGCTTACATGCCTTCAGT | 161009 | 161027 | 109 |
| 435324 | rs362322 | Minor (10) | CAGCTTACACGCCTTCAGT | 161009 | 161027 | 110 |
| 435868 | rs362275 | Major (10) | AAGAAGCCTGATAAAATCT | 164246 | 164264 | 111 |
| 435893 | rs362275 | Minor (10) | AAGAAGCCTAATAAAATCT | 164246 | 164264 | 112 |
| 435343 | rs2276881 | Major (8) | CATACATCAGCTCAAACTG | 171303 | 171321 | 113 |
| 435361 | rs2276881 | Minor (8) | CATACATTAGCTCAAACTG | 171303 | 171321 | 114 |
| 435307 | rs2276881 | Major (10) | CACATACATCAGCTCAAAC | 171305 | 171323 | 115 |
| 435325 | rs2276881 | Minor (10) | CACATACATTAGCTCAAAC | 171305 | 171323 | 116 |
| 435368 | rs2276881 | Major (12) | GTCACATACATCAGCTCAA | 171307 | 171325 | 117 |
| 435866 | rs3121419 | Major (10) | GAGACTATAGCACCCAGAT | 171901 | 171919 | 118 |
| 435891 | rs3121419 | Minor (10) | GAGACTATAACACCCAGAT | 171901 | 171919 | 119 |
| 435344 | rs362272 | Major (8) | TAGAGGACGCCGTGCAGGG | 174622 | 174640 | 120 |
| 435362 | rs362272 | Minor (8) | TAGAGGATGCCGTGCAGGG | 174622 | 174640 | 121 |
| 435308 | rs362272 | Major (10) | CATAGAGGACGCCGTGCAG | 174624 | 174642 | 122 |
| 435326 | rs362272 | Minor (10) | CATAGAGGATGCCGTGCAG | 174624 | 174642 | 123 |
| 435369 | rs362272 | Major (12) | CACATAGAGGACGCCGTGC | 174626 | 174644 | 124 |
| 435867 | rs362271 | Major (10) | ACGTGTGTACAGAACCTGC | 175162 | 175180 | 125 |
| 435892 | rs362271 | Minor (10) | ACGTGTGTATAGAACCTGC | 175162 | 175180 | 126 |
| 435873 | rs3775061 | Major (10) | TGTTCAGAATGCCTCATCT | 178398 | 178416 | 127 |
| 435898 | rs3775061 | Minor (10) | TGTTCAGAACGCCTCATCT | 178398 | 178416 | 128 |
| 435345 | rs362310 | Major (8) | AAACGGCGCAGCGGGAAGG | 179418 | 179436 | 129 |
| 435363 | rs362310 | Minor (8) | AAACGGCACAGCGGGAAGG | 179418 | 179436 | 130 |
| 435309 | rs362310 | Major (10) | AGAAACGGCGCAGCGGGAA | 179420 | 179438 | 131 |
| 435327 | rs362310 | Minor (10) | AGAAACGGCACAGCGGGAA | 179420 | 179438 | 132 |
| 435915 | rs362307 | Major (6) | AGGGCGCAGACTTCCAAAG | 181485 | 181503 | 133 |
| 435927 | rs362307 | Minor (6) | AGGGCACAGACTTCCAAAG | 181485 | 181503 | 134 |
| 435917 | rs362307 | Major (7) | AAGGGCGCAGACTTCCAAA | 181486 | 181504 | 135 |
| 435929 | rs362307 | Minor (7) | AAGGGCACAGACTTCCAAA | 181486 | 181504 | 136 |
| 435346 | rs362307 | Major (8) | CAAGGGCGCAGACTTCCAA | 181487 | 181505 | 137 |
| 435364 | rs362307 | Minor (8) | CAAGGGCACAGACTTCCAA | 181487 | 181505 | 138 |
| 435919 | rs362307 | Major (9) | ACAAGGGCGCAGACTTCCA | 181488 | 181506 | 139 |
| 435931 | rs362307 | Minor (9) | ACAAGGGCACAGACTTCCA | 181488 | 181506 | 140 |
| 435310 | rs362307 | Major (10) | CACAAGGGCGCAGACTTCC | 181489 | 181507 | 141 |
| 435328 | rs362307 | Minor (10) | CACAAGGGCACAGACTTCC | 181489 | 181507 | 142 |
| 435921 | rs362307 | Major (11) | GCACAAGGGCGCAGACTTC | 181490 | 181508 | 143 |
| 435933 | rs362307 | Minor (11) | GCACAAGGGCACAGACTTC | 181490 | 181508 | 144 |
| 435370 | rs362307 | Major (12) | GGCACAAGGGCGCAGACTT | 181491 | 181509 | 145 |
| 435925 | rs362307 | Minor (12) | GGCACAAGGGCACAGACTT | 181491 | 181509 | 146 |
| 435923 | rs362307 | Major (14) | AGGGCACAAGGGCGCAGAC | 181493 | 181511 | 147 |
| 435935 | rs362307 | Minor (14) | AGGGCACAAGGGCACAGAC | 181493 | 181511 | 148 |
| 435869 | rs362306 | Major (10) | GAGCAGCTGCAACCTGGCA | 181744 | 181762 | 149 |
| 435894 | rs362306 | Minor (10) | GAGCAGCTGTAACCTGGCA | 181744 | 181762 | 150 |
| 435347 | rs362303 | Major (8) | TGGTGCCGGGTGTCTAGCA | 181949 | 181967 | 151 |
| 435365 | rs362303 | Minor (8) | TGGTGCCAGGTGTCTAGCA | 181949 | 181967 | 152 |
| 435311 | rs362303 | Major (10) | AATGGTGCCGGGTGTCTAG | 181951 | 181969 | 153 |
| 435329 | rs362303 | Minor (10) | AATGGTGCCAGGTGTCTAG | 181951 | 181969 | 154 |
| 435882 | rs362296 | Major (10) | GGGGACAGGGTGTGCTCTC | 186651 | 186669 | 155 |
| 435907 | rs362296 | Minor (10) | GGGGACAGGTTGTGCTCTC | 186651 | 186669 | 156 |

TABLE 4
Comparison of inhibition of HTT mRNA levels by ISIS 387916
and ISIS 388816 with that by chimeric oligonucleotides
targeting SNP positions on the HTT gene (SEQ ID NO: 1)

| ISIS | SNP | Target | % inhibition | | | SEQ |
|---|---|---|---|---|---|---|
| No | RS No. | allele | GM04281 | GM02171 | GM02173B | ID NO |
| 387916 | n/a | n/a | 96 | 96 | 98 | 6 |
| 388816 | n/a | n/a | 76 | 88 | 85 | 7 |
| 435330 | rs3856973 | Major (8) | 64 | 51 | 36 | 8 |
| 435348 | rs3856973 | Minor (8) | 50 | 88 | 80 | 9 |
| 435294 | rs3856973 | Major (10) | 54 | 46 | 54 | 10 |
| 435312 | rs3856973 | Minor (10) | 20 | 82 | 58 | 11 |
| 435864 | rs2285086 | Major (10) | 54 | 28 | 26 | 12 |
| 435889 | rs2285086 | Minor (10) | 17 | 43 | 41 | 13 |
| 435878 | rs7659144 | Major (10) | 43 | 32 | 39 | 14 |
| 435903 | rs7659144 | Minor (10) | 16 | 37 | 29 | 15 |
| 435863 | rs16843804 | Major (10) | 63 | 78 | 81 | 16 |
| 435888 | rs16843804 | Minor (10) | 58 | 75 | 77 | 17 |
| 435331 | rs2024115 | Major (8) | 56 | 27 | 56 | 18 |
| 435349 | rs2024115 | Minor (8) | 26 | 91 | 66 | 19 |
| 435295 | rs2024115 | Major (10) | 53 | 57 | 62 | 20 |
| 435313 | rs2024115 | Minor (10) | 25 | 87 | 53 | 21 |
| 435862 | rs10015979 | Major (10) | 8 | 51 | 40 | 22 |
| 435887 | rs10015979 | Minor (10) | 40 | 22 | 28 | 23 |
| 435880 | rs7691627 | Major (10) | 43 | 17 | 21 | 24 |
| 435905 | rs7691627 | Minor (10) | 13 | 27 | 15 | 25 |
| 435885 | rs2798235 | Major (10) | 38 | 39 | 30 | 26 |
| 435910 | rs2798235 | Minor (10) | 17 | 30 | 16 | 27 |
| 435874 | rs4690072 | Major (10) | 61 | 34 | 48 | 28 |
| 435899 | rs4690072 | Minor (10) | 50 | 41 | 45 | 29 |
| 435875 | rs6446723 | Major (10) | 28 | 13 | 35 | 30 |
| 435900 | rs6446723 | Minor (10) | 24 | 56 | 37 | 31 |
| 435332 | rs363081 | Major (8) | 76 | 95 | 88 | 32 |
| 435350 | rs363081 | Minor (8) | 27 | 61 | 43 | 33 |
| 435296 | rs363081 | Major (10) | 59 | 77 | 66 | 34 |
| 435314 | rs363081 | Minor (10) | 38 | 66 | 40 | 35 |
| 435886 | rs363080 | Major (10) | 74 | 72 | 79 | 36 |
| 435911 | rs363080 | Minor (10) | 57 | 58 | 54 | 37 |
| 435914 | rs363075 | Major (6) | 95 | 92 | 95 | 38 |
| 435926 | rs363075 | Minor (6) | 88 | 81 | 79 | 39 |
| 435916 | rs363075 | Major (7) | 90 | 92 | 94 | 40 |
| 435928 | rs363075 | Minor (7) | 83 | 79 | 85 | 41 |
| 435333 | rs363075 | Major (8) | 86 | 97 | 91 | 42 |
| 435351 | rs363075 | Minor (8) | 59 | 80 | 58 | 43 |
| 435918 | rs363075 | Major (9) | 83 | 90 | 91 | 44 |
| 435930 | rs363075 | Minor (9) | 29 | 49 | 49 | 45 |
| 435297 | rs363075 | Major (10) | 74 | 84 | 83 | 46 |
| 435315 | rs363075 | Minor (10) | 47 | 63 | 45 | 47 |
| 435920 | rs363075 | Major (11) | 78 | 66 | 83 | 48 |
| 435932 | rs363075 | Minor (11) | 39 | 20 | 19 | 49 |
| 435366 | rs363075 | Major (12) | 80 | 91 | 85 | 50 |
| 435924 | rs363075 | Minor (12) | 37 | 49 | 58 | 51 |
| 435922 | rs363075 | Major (14) | 80 | 90 | 91 | 52 |
| 435934 | rs363075 | Minor (14) | 63 | 70 | 80 | 53 |
| 435334 | rs363064 | Major (8) | 50 | 59 | 44 | 54 |
| 435352 | rs363064 | Minor (8) | 12 | 37 | 48 | 55 |
| 435298 | rs363064 | Major (10) | 81 | 92 | 87 | 56 |
| 435316 | rs363064 | Minor (10) | 69 | 90 | 80 | 57 |
| 435335 | rs3025849 | Major (8) | 0 | 40 | 37 | 58 |
| 435353 | rs3025849 | Minor (8) | 0 | 29 | 18 | 59 |
| 435299 | rs3025849 | Major (10) | 0 | 34 | 67 | 60 |
| 435317 | rs3025849 | Minor (10) | 0 | 38 | 34 | 61 |
| 435877 | rs6855981 | Major (10) | 31 | 59 | 58 | 62 |
| 435902 | rs6855981 | Minor (10) | 0 | 43 | 27 | 63 |
| 435336 | rs363102 | Major (8) | 0 | 21 | 19 | 64 |
| 435354 | rs363102 | Minor (8) | 0 | 36 | 33 | 65 |
| 435300 | rs363102 | Major (10) | 0 | 34 | 24 | 66 |
| 435318 | rs363102 | Minor (10) | 0 | 30 | 20 | 67 |
| 435884 | rs11731237 | Major (10) | 7 | 46 | 51 | 68 |
| 435909 | rs11731237 | Minor (10) | 30 | 47 | 41 | 69 |
| 435337 | rs4690073 | Major (8) | 12 | 0 | 12 | 70 |
| 435355 | rs4690073 | Minor (8) | 0 | 26 | 33 | 71 |
| 435301 | rs4690073 | Major (10) | 23 | 0 | 10 | 72 |
| 435319 | rs4690073 | Minor (10) | 0 | 45 | 53 | 73 |
| 435883 | rs363144 | Major (10) | 24 | 23 | 39 | 74 |
| 435908 | rs363144 | Minor (10) | 27 | 20 | 22 | 75 |
| 435338 | rs3025838 | Major (8) | 31 | 46 | 69 | 76 |
| 435356 | rs3025838 | Minor (8) | 3 | 25 | 17 | 77 |
| 435302 | rs3025838 | Major (10) | 39 | 73 | 67 | 78 |
| 435320 | rs3025838 | Minor (10) | 21 | 49 | 32 | 79 |
| 435339 | rs363099 | Major (8) | 84 | 87 | 76 | 80 |
| 435357 | rs363099 | Minor (8) | 71 | 91 | 90 | 81 |
| 435303 | rs363099 | Major (10) | 83 | 92 | 85 | 82 |
| 435321 | rs363099 | Minor (10) | 84 | 95 | 89 | 83 |
| 435367 | rs363099 | Major (12) | 76 | 82 | 72 | 84 |
| 435340 | rs363096 | Major (8) | 0 | 47 | 52 | 85 |
| 435358 | rs363096 | Minor (8) | 0 | 25 | 35 | 86 |
| 435304 | rs363096 | Major (10) | 5 | 33 | 36 | 87 |
| 435322 | rs363096 | Minor (10) | 2 | 30 | 32 | 88 |
| 435341 | rs2298967 | Major (8) | 54 | 72 | 56 | 89 |
| 435359 | rs2298967 | Minor (8) | 25 | 59 | 63 | 90 |
| 435305 | rs2298967 | Major (10) | 66 | 80 | 78 | 91 |
| 435323 | rs2298967 | Minor (10) | 36 | 79 | 66 | 92 |
| 435865 | rs2298969 | Major (10) | 53 | 72 | 79 | 93 |
| 435890 | rs2298969 | Minor (10) | 65 | 46 | 54 | 94 |
| 435876 | rs6844859 | Major (10) | 70 | 67 | 77 | 95 |
| 435901 | rs6844859 | Minor (10) | 39 | 83 | 80 | 96 |
| 435872 | rs363092 | Major (10) | 46 | 41 | 54 | 97 |
| 435897 | rs363092 | Minor (10) | 37 | 69 | 57 | 98 |
| 435879 | rs7685686 | Major (10) | 83 | 31 | 70 | 99 |
| 435904 | rs7685686 | Minor (10) | 30 | 92 | 72 | 100 |
| 435871 | rs363088 | Major (10) | 70 | 55 | 70 | 101 |
| 435896 | rs363088 | Minor (10) | 66 | 74 | 80 | 102 |
| 435870 | rs362331 | Major (10) | 88 | 74 | 88 | 103 |
| 435895 | rs362331 | Minor (10) | 78 | 92 | 86 | 104 |
| 435881 | rs916171 | Major (10) | 0 | 57 | 51 | 105 |
| 435906 | rs916171 | Minor (10) | 14 | 26 | 17 | 106 |
| 435342 | rs362322 | Major (8) | 47 | 74 | 67 | 107 |
| 435360 | rs362322 | Minor (8) | 17 | 58 | 52 | 108 |
| 435306 | rs362322 | Major (10) | 50 | 77 | 65 | 109 |
| 435324 | rs362322 | Minor (10) | 42 | 61 | 64 | 110 |
| 435868 | rs362275 | Major (10) | 54 | 35 | 43 | 111 |
| 435893 | rs362275 | Minor (10) | 3 | 27 | 33 | 112 |
| 435343 | rs2276881 | Major (8) | 59 | 76 | 65 | 113 |
| 435361 | rs2276881 | Minor (8) | 58 | 44 | 20 | 114 |
| 435307 | rs2276881 | Major (10) | 69 | 82 | 81 | 115 |
| 435325 | rs2276881 | Minor (10) | 17 | 47 | 43 | 116 |
| 435368 | rs2276881 | Major (12) | 84 | 96 | 92 | 117 |
| 435866 | rs3121419 | Major (10) | 67 | 61 | 64 | 118 |
| 435891 | rs3121419 | Minor (10) | 53 | 76 | 73 | 119 |
| 435344 | rs362272 | Major (8) | 35 | 46 | 36 | 120 |
| 435362 | rs362272 | Minor (8) | 34 | 68 | 57 | 121 |
| 435308 | rs362272 | Major (10) | 26 | 30 | 35 | 122 |
| 435326 | rs362272 | Minor (10) | 29 | 50 | 39 | 123 |
| 435369 | rs362272 | Major (12) | 66 | 74 | 65 | 124 |
| 435867 | rs362271 | Major (10) | 73 | 74 | 75 | 125 |
| 435892 | rs362271 | Minor (10) | 52 | 74 | 79 | 126 |
| 435873 | rs3775061 | Major (10) | 40 | 32 | 47 | 127 |
| 435898 | rs3775061 | Minor (10) | 13 | 20 | 24 | 128 |
| 435345 | rs362310 | Major (8) | 38 | 55 | 52 | 129 |
| 435363 | rs362310 | Minor (8) | 45 | 67 | 60 | 130 |
| 435309 | rs362310 | Major (10) | 33 | 44 | 56 | 131 |
| 435327 | rs362310 | Minor (10) | 33 | 71 | 61 | 132 |
| 435915 | rs362307 | Major (6) | 61 | 54 | 58 | 133 |
| 435927 | rs362307 | Minor (6) | 31 | 35 | 44 | 134 |
| 435917 | rs362307 | Major (7) | 67 | 76 | 66 | 135 |
| 435929 | rs362307 | Minor (7) | 33 | 34 | 55 | 136 |
| 435346 | rs362307 | Major (8) | 67 | 89 | 66 | 137 |
| 435364 | rs362307 | Minor (8) | 46 | 72 | 66 | 138 |
| 435919 | rs362307 | Major (9) | 84 | 79 | 70 | 139 |
| 435931 | rs362307 | Minor (9) | 74 | 74 | 86 | 140 |
| 435310 | rs362307 | Major (10) | 74 | 81 | 71 | 141 |
| 435328 | rs362307 | Minor (10) | 47 | 69 | 75 | 142 |
| 435921 | rs362307 | Major (11) | 74 | 77 | 69 | 143 |
| 435933 | rs362307 | Minor (11) | 38 | 47 | 74 | 144 |
| 435370 | rs362307 | Major (12) | 64 | 74 | 38 | 145 |
| 435925 | rs362307 | Minor (12) | 60 | 66 | 80 | 146 |
| 435923 | rs362307 | Major (14) | 73 | 66 | 71 | 147 |
| 435935 | rs362307 | Minor (14) | 68 | 75 | 87 | 148 |
| 435869 | rs362306 | Major (10) | 82 | 77 | 81 | 149 |
| 435894 | rs362306 | Minor (10) | 28 | 79 | 72 | 150 |
| 435347 | rs362303 | Major (8) | 68 | 74 | 71 | 151 |
| 435365 | rs362303 | Minor (8) | 69 | 83 | 76 | 152 |
| 435311 | rs362303 | Major (10) | 46 | 56 | 72 | 153 |
| 435329 | rs362303 | Minor (10) | 49 | 62 | 39 | 154 |
| 435882 | rs362296 | Major (10) | 29 | 48 | 56 | 155 |
| 435907 | rs362296 | Minor (10) | 42 | 56 | 52 | 156 |

### Example 3: Dose-Dependent Antisense Inhibition of Human Huntingtin mRNA Levels in Coriell Fibroblast Cell Lines

Gapmers from the study described in Example 2 were selected and tested at various doses in GM04281, GM02171, and GM02173B cell lines. Each cell line was plated at a density of 25,000 cells per well and transfected using electroporation with 750 nM, 1,500 nM, 3,000 nM, 6,000 nM, and 12,000 nM concentrations of antisense oligonucleotide, as specified in Table 5, 6, and 7. After a treatment period of approximately 16 hours, RNA was isolated from the cells and HTT mRNA levels were measured by quantitative real-time PCR. Human HTT primer probe set RTS2617 was used to measure mRNA levels. HTT mRNA levels were adjusted according to total RNA content, as measured by RIBOGREEN. Results are presented as percent inhibition of HTT mRNA, relative to untreated control cells. IC₅₀ values are also provided in Tables 5, 6, and 7.

TABLE 5
Dose-dependent antisense inhibition
of human HTT in GM04281 cells

| ISIS | 750 | 1,500 | 3,000 | 6,000 | 12,000 | IC₅₀ |
|---|---|---|---|---|---|---|
| No. | nM | nM | nM | nM | nM | (μM) |
| 387916 | 51 | 81 | 80 | 91 | 97 | 0.6 |
| 435330 | 24 | 49 | 50 | 73 | 85 | 2.5 |
| 435331 | 23 | 38 | 64 | 72 | 74 | 2.4 |
| 435868 | 3 | 17 | 7 | 29 | 63 | 6.7 |
| 435870 | 53 | 73 | 77 | 86 | 93 | 0.6 |
| 435871 | 28 | 51 | 52 | 78 | 89 | 1.7 |
| 435874 | 14 | 21 | 28 | 64 | 82 | 3.3 |
| 435879 | 42 | 57 | 57 | 81 | 91 | 1.1 |
| 435890 | 48 | 56 | 62 | 76 | 91 | 0.9 |
| 435929 | 10 | 0 | 5 | 12 | 48 | 13.8 |
| 435931 | 20 | 17 | 53 | 62 | 81 | 2.9 |
| 435933 | 0 | 7 | 24 | 43 | 49 | 10.7 |
| 435935 | 0 | 38 | 38 | 62 | 29 | 4.2 |

TABLE 6
Dose-dependent antisense inhibition
of human HTT in GM02171 cells

| ISIS | 750 | 1,500 | 3.000 | 6,000 | 12,000 | IC₅₀ |
|---|---|---|---|---|---|---|
| No. | nM | nM | nM | nM | nM | (μM) |
| 387916 | 57 | 73 | 81 | 93 | 98 | 0.4 |
| 435330 | 27 | 37 | 0 | 44 | 63 | 4.4 |
| 435331 | 35 | 34 | 19 | 41 | 63 | 3.5 |
| 435868 | 21 | 21 | 39 | 24 | 12 | >12.0 |
| 435870 | 50 | 53 | 57 | 70 | 79 | 0.9 |
| 435871 | 32 | 46 | 45 | 58 | 62 | 3.9 |
| 435874 | 1 | 0 | 4 | 11 | 6 | >12.0 |
| 435879 | 32 | 14 | 17 | 45 | 38 | >12.0 |
| 435890 | 34 | 33 | 40 | 51 | 62 | 5.4 |
| 435929 | 25 | 22 | 31 | 5 | 29 | >12.0 |
| 435931 | 15 | 28 | 27 | 60 | 79 | 3.7 |
| 435933 | 13 | 36 | 21 | 43 | 48 | 12.2 |
| 435935 | 25 | 42 | 27 | 61 | 68 | 3.2 |

TABLE 7
Dose-dependent antisense inhibition
of human HTT in GM02173B cells

| ISIS | 750 | 1,500 | 3,000 | 6.000 | 12,000 | IC₅₀ |
|---|---|---|---|---|---|---|
| No. | nM | nM | nM | nM | nM | (μM) |
| 387916 | 43 | 67 | 80 | 86 | 97 | 1.1 |
| 435330 | 22 | 21 | 0 | 52 | 62 | 5.3 |
| 435331 | 19 | 17 | 32 | 50 | 55 | 9.4 |
| 435868 | 17 | 25 | 41 | 13 | 26 | >12.0 |
| 435870 | 24 | 57 | 70 | 78 | 75 | 1.8 |
| 435871 | 8 | 30 | 42 | 50 | 48 | 5.0 |
| 435874 | 31 | 35 | 28 | 35 | 42 | >12.0 |
| 435879 | 39 | 44 | 42 | 60 | 64 | 2.5 |
| 435890 | 38 | 36 | 50 | 65 | 73 | 3.1 |
| 435929 | 19 | 17 | 19 | 42 | 35 | 7.7 |
| 435931 | 40 | 19 | 31 | 48 | 71 | 5.8 |
| 435933 | 35 | 24 | 47 | 52 | 59 | 4.4 |
| 435935 | 25 | 23 | 40 | 73 | 77 | 3.7 |

### Example 4: Dose-Dependent Antisense Inhibition of Human Huntingtin mRNA Levels in Coriell Fibroblast Cell Lines

Gapmers from the study described in Example 2 were selected and tested at various doses in GM04281, GM02171, and GM02173B cell lines. Each cell line was plated at a density of 25,000 cells per well and transfected using electroporation with 750 nM, 1,500 nM, 3,000 nM, 6,000 nM, and 12,000 nM concentrations of antisense oligonucleotide, as specified in Table 8, 9, and 10. After a treatment period of approximately 16 hours, RNA was isolated from the cells and HTT mRNA levels were measured by quantitative real-time PCR. Human HTT primer probe set RTS2617 was used to measure mRNA levels. HTT mRNA levels were adjusted according to total RNA content, as measured by RIBOGREEN. Results are presented as percent inhibition of HTT mRNA relative to untreated control cells. IC₅₀ values are also provided in Tables 8, 9, and 10.

TABLE 8
Dose-dependent antisense inhibition
of human HTT in GM04281 cells

| ISIS | 750 | 1,500 | 3,000 | 6,000 | 12,000 | IC₅₀ |
|---|---|---|---|---|---|---|
| No. | nM | nM | nM | nM | nM | (μM) |
| 387916 | 61 | 78 | 90 | 94 | 97 | <0.8 |
| 435303 | 33 | 39 | 69 | 79 | 91 | 1.5 |
| 435328 | 0 | 12 | 16 | 51 | 75 | 5.3 |
| 435331 | 27 | 48 | 48 | 70 | 82 | 2.1 |
| 435339 | 46 | 37 | 61 | 73 | 89 | 2.3 |
| 435869 | 17 | 35 | 44 | 66 | 80 | 3.3 |
| 435870 | 44 | 60 | 64 | 84 | 84 | 1.1 |
| 435871 | 41 | 50 | 71 | 78 | 87 | 1.2 |
| 435874 | 24 | 36 | 35 | 65 | 73 | 3.1 |
| 435879 | 46 | 52 | 78 | 81 | 92 | 0.9 |
| 435890 | 41 | 53 | 63 | 80 | 86 | 1.3 |
| 435925 | 0 | 14 | 39 | 60 | 87 | 4.2 |
| 435926 | 20 | 28 | 67 | 81 | 89 | 2.0 |
| 435928 | 32 | 49 | 73 | 86 | 86 | 1.8 |
| 435931 | 22 | 24 | 40 | 59 | 90 | 3.8 |

TABLE 9
Dose-dependent antisense inhibition
of human HTT in GM02171 cells

| ISIS | 750 | 1,500 | 3,000 | 6,000 | 12,000 | IC₅₀ |
|---|---|---|---|---|---|---|
| No. | nM | nM | nM | nM | nM | (μM) |
| 387916 | 50 | 64 | 90 | 95 | 96 | 0.7 |
| 435303 | 14 | 32 | 68 | 79 | 85 | 2.8 |
| 435328 | 0 | 12 | 20 | 38 | 55 | 10.3 |
| 435331 | 0 | 13 | 5 | 30 | 36 | >12.0 |
| 435339 | 30 | 40 | 58 | 63 | 49 | 2.5 |
| 435869 | 13 | 25 | 31 | 47 | 87 | 4.0 |
| 435870 | 18 | 31 | 44 | 66 | 74 | 3.5 |
| 435871 | 1 | 20 | 29 | 49 | 64 | 6.5 |
| 435874 | 3 | 6 | 12 | 17 | 31 | >12.0 |
| 435879 | 0 | 2 | 12 | 35 | 44 | >12.0 |
| 435890 | 15 | 16 | 30 | 48 | 72 | 5.8 |
| 435925 | 0 | 0 | 22 | 48 | 29 | 6.3 |
| 435926 | 25 | 28 | 58 | 74 | 85 | 2.3 |
| 435928 | 18 | 53 | 61 | 86 | 83 | 2.5 |
| 435931 | 0 | 4 | 25 | 46 | 68 | 6.7 |

TABLE 10
Dose-dependent antisense inhibition
of human HTT in GM02173B cells

| ISIS | 750 | 1,500 | 3,000 | 6,000 | 12,000 | IC₅₀ |
|---|---|---|---|---|---|---|
| No. | nM | nM | nM | nM | nM | (μM) |
| 387916 | 27 | 65 | 84 | 81 | 96 | 1.9 |
| 435303 | 23 | 48 | 52 | 76 | 76 | 2.9 |
| 435328 | 8 | 14 | 19 | 34 | 50 | 15.7 |
| 435331 | 10 | 17 | 16 | 27 | 32 | >12.0 |
| 435339 | 28 | 26 | 38 | 67 | 82 | 3.8 |
| 435869 | 12 | 24 | 37 | 45 | 79 | 4.2 |
| 435870 | 20 | 26 | 58 | 53 | 78 | 2.7 |
| 435871 | 15 | 16 | 32 | 45 | 71 | 6.0 |
| 435874 | 13 | 8 | 28 | 36 | 31 | >12.0 |
| 435879 | 22 | 20 | 36 | 53 | 60 | 6.0 |
| 435890 | 21 | 28 | 34 | 54 | 71 | 4.3 |
| 435925 | 2 | 10 | 28 | 43 | 78 | 5.9 |
| 435926 | 7 | 25 | 37 | 73 | 79 | 3.5 |
| 435928 | 15 | 39 | 60 | 73 | 87 | 2.5 |
| 435931 | 13 | 13 | 32 | 61 | 62 | 6.7 |

### Example 5: Antisense Inhibition of Human HTT in GM04281 Cells

Additional antisense oligonucleotides were designed based on the gapmers selected from studies described in Example 4. These oligonucleotides were designed by creating gapmers shifted slightly upstream and downstream (i.e. “microwalk”) of the original gapmers from Tables 8, 9, and 10. Antisense oligonucleotides were also created with uniform MOE, as well as with various motifs, 2-9-6 MOE, 3-9-3 MOE, 3-9-4 MOE, 3-9-5 MOE, 4-10-5 MOE, 4-11-4 MOE, 4-7-4 MOE, 4-9-4 MOE, 4-9-5 MOE, 5-10-4 MOE, 5-7-5 MOE, 5-8-6 MOE, 5-9-3 MOE, 5-9-5 MOE, 6-7-6 MOE, 6-9-2 MOE, and 6-8-5 MOE.

In addition, antisense oligonucleotides were designed targeting SNP RS Nos. rs2857936, rs12506200, rs762855, and rs1006798 (refer to Table 2). The oligonucleotides were designed targeting either the major allele or the minor allele, and with the SNP position opposite either position 8 or position 10 of the gapmer.

These gapmers were tested in vitro. Cultured GM04281 cells at a density of 25,000 cells per well were transfected using electroporation with 10,000 nM antisense oligonucleotide. After a treatment period of approximately 24 hours, RNA was isolated from the cells and HTT mRNA levels were measured by quantitative real-time PCR. HTT mRNA levels were adjusted according to total RNA content, as measured by RIBOGREEN. Results are presented in Tables 11-19 as percent inhibition of HTT mRNA, relative to untreated control cells.

The gapmers, ISIS 435869, ISIS 435870, ISIS 435874, ISIS 435879, and ISIS 435890, from which some of the newly designed gapmers were derived are marked with an asterisk (*) in the table. ISIS 387916 was included in the study as a benchmark oligonucleotide against which the potency of the antisense oligonucleotides targeting nucleotides overlapping each SNP position could be compared.

The uniform MOE oligonucleotides are 15 nucleotides in length.

The 2-9-6 gapmers are 17 nucleotides in length, wherein the central gap segment is comprised of nine 2′-deoxynucleotides and is flanked on the 5′ direction by a wing comprising 2 nucleotides and on the 3′ direction by a wing comprising 6 nucleotides.

The 3-9-3 gapmers are 15 nucleotides in length, wherein the central gap segment is comprised of nine 2′-deoxynucleotides and is flanked on both 5′ and 3′ directions by wings comprising 3 nucleotides each.

The 3-9-4 gapmers are 16 nucleotides in length, wherein the central gap segment is comprised of nine 2′-deoxynucleotides and is flanked on the 5′ direction by a wing comprising 3 nucleotides and on the 3′ direction by a wing comprising 4 nucleotides.

The 3-9-5 gapmers are 17 nucleotides in length, wherein the central gap segment is comprised of nine 2′-deoxynucleotides and is flanked on the 5′ direction by a wing comprising 3 nucleotides and on the 3′ direction by a wing comprising 5 nucleotides.

The 4-10-5 gapmers are 19 nucleotides in length, wherein the central gap segment is comprised of ten 2′-deoxynucleotides and is flanked on the 5′ direction by a wing comprising 4 nucleotides and on the 3′ direction by a wing comprising 5 nucleotides.

The 4-11-4 gapmers are 19 nucleotides in length, wherein the central gap segment is comprised of eleven 2′-deoxynucleotides and is flanked on both 5′ and 3′ directions by wings comprising 4 nucleotides each.

The 4-7-4 gapmers are 15 nucleotides in length, wherein the central gap segment is comprised of seven 2′-deoxynucleotides and is flanked on both 5′ and 3′ directions by wings comprising 4 nucleotides each.

The 4-9-4 gapmers are 17 nucleotides in length, wherein the central gap segment is comprised of nine 2′-deoxynucleotides and is flanked on both 5′ and 3′ directions by wings comprising 4 nucleotides each.

The 4-9-5 gapmers are 18 nucleotides in length, wherein the central gap segment is comprised of nine 2′-deoxynucleotides and is flanked on the 5′ direction by a wing comprising 4 nucleotides and on the 3′ direction by a wing comprising 5 nucleotides.

The 5-10-4 gapmers are 19 nucleotides in length, wherein the central gap segment is comprised of ten 2′-deoxynucleotides and is flanked on the 5′ direction by a wing comprising 5 nucleotides and on the 3′ direction by a wing comprising 4 nucleotides.

The 5-7-5 gapmers are 17 nucleotides in length, wherein the central gap segment is comprised of seven 2′-deoxynucleotides and is flanked on both 5′ and 3′ directions by wings comprising 5 nucleotides each.

The 5-8-6 gapmers are 19 nucleotides in length, wherein the central gap segment is comprised of eight 2′-deoxynucleotides and is flanked on the 5′ direction by a wing comprising 5 nucleotides and on the 3′ direction by a wing comprising 6 nucleotides.

The 5-9-3 gapmers are 17 nucleotides in length, wherein the central gap segment is comprised of nine 2′-deoxynucleotides and is flanked on the 5′ direction by a wing comprising 5 nucleotides and on the 3′ direction by a wing comprising 3 nucleotides.

The 5-9-5 gapmers are 19 nucleotides in length, wherein the central gap segment is comprised of nine 2′-deoxynucleotides and is flanked on both 5′ and 3′ directions by wings comprising 5 nucleotides each.

The 6-7-6 gapmers are 19 nucleotides in length, wherein the central gap segment is comprised of seven 2′-deoxynucleotides and is flanked on both 5′ and 3′ directions by wings comprising 6 nucleotides each.

The 6-9-2 gapmers are 17 nucleotides in length, wherein the central gap segment is comprised of nine 2′-deoxynucleotides and is flanked on the 5′ direction by a wing comprising 6 nucleotides and on the 3′ direction by a wing comprising 2 nucleotides.

The 6-8-5 gapmers are 19 nucleotides in length, wherein the central gap segment is comprised of eight 2′-deoxynucleotides and is flanked on the 5′ direction by a wing comprising 6 nucleotides and on the 3′ direction by a wing comprising 5 nucleotides.

For each of the motifs, each nucleotide in the 5′ wing segment and each nucleotide in the 3′ wing segment has a 2′-MOE modification. The internucleoside linkages throughout each gapmer are phosphorothioate (P═S) linkages. All cytosine nucleobases throughout each gapmer are 5-methylcytosines.

The oligonucleotides are organized in tables according to the SNP they target. “Start site” indicates the 5′-most nucleotide to which the gapmer is targeted. “Stop site” indicates the 3′-most nucleotide to which the gapmer is targeted. ‘Target allele’ indicates whether the gapmer is targeted to the major or the minor allele. The number in parentheses indicates the position on the oligonucleotide opposite to the SNP position.

TABLE 11
Comparison of inhibition of human HTT mRNA levels by ISIS 387916 and chimeric antisense
oligonucleotides targeted to SNP rs2857936 (nucleobases 1952 to 1972 of SEQ ID NO: 1)

| | | | | | | | SEQ |
|---|---|---|---|---|---|---|---|
| Start | Stop | Target | ISIS | | | % | ID |
| Site | Site | allele | No. | Sequence | Motif | inhibition | NO |
| 145466 | 145485 | n/a | 387916 | TCTCTATTGCACATTCCAA | 5-10-5 | 98 | 6 |
| | | | | G | | | |
| 1952 | 1970 | Minor (8) | 459908 | GCTTTTCATTGAAAAGAA | 5-9-5 | 26 | 157 |
| | | | | A | | | |
| 1952 | 1970 | Major (8) | 459916 | GCTTTTCGTTGAAAAGAA | 5-9-5 | 8 | 158 |
| | | | | A | | | |
| 1954 | 1972 | Minor (10) | 459904 | CTGCTTTTCATTGAAAAGA | 5-9-5 | 23 | 159 |
| 1954 | 1972 | Major (10) | 459912 | CTGCTTTTCGTTGAAAAGA | 5-9-5 | 8 | 160 |

TABLE 12
Comparison of inhibition of human HTT mRNA levels by ISIS 387916 and chimeric antisense
oligonucleotides targeted to SNP rs12506200 (nucleobases 3695 to 3715 of SEQ ID NO: 1)

| | | | | | | | SEQ |
|---|---|---|---|---|---|---|---|
| Start | Stop | Target | ISIS | | | % | ID |
| Site | Site | allele | No. | Sequence | Motif | inhibition | NO |
| 145466 | 145485 | n/a | 387916 | TCTCTATTGCACATTCCAA | 5-10-5 | 98 | 6 |
| | | | | G | | | |
| 3695 | 3713 | Major (8) | 459909 | ACTAGGCCGGGCATGCTG | 5-9-5 | 48 | 161 |
| | | | | G | | | |
| 3695 | 3713 | Minor (8) | 459917 | ACTAGGCTGGGCATGCTG | 5-9-5 | 35 | 162 |
| | | | | G | | | |
| 3697 | 3715 | Major (10) | 459905 | AGACTAGGCCGGGCATGC | 5-9-5 | 33 | 163 |
| | | | | T | | | |
| 3697 | 3715 | Minor (10) | 459913 | AGACTAGGCTGGGCATGC | 5-9-5 | 45 | 164 |
| | | | | T | | | |

TABLE 13
Comparison of inhibition of human HTT mRNA levels by ISIS 387916 and chimeric antisense
oligonucleotides targeted to SNP rs762855 (nucleobases 14437 to 14457 of SEQ ID NO: 1)

| | | | | | | | SEQ |
|---|---|---|---|---|---|---|---|
| Start | Stop | Target | ISIS | | | % | ID |
| Site | Site | allele | No. | Sequence | Motif | inhibition | NO |
| 145466 | 145485 | n/a | 387916 | TCTCTATTGCACATTCCAA | 5-10-5 | 98 | 6 |
| | | | | G | | | |
| 14437 | 14455 | Minor (8) | 459910 | AAACAGCTGTTAGTTCCC | 5-9-5 | 27 | 165 |
| | | | | A | | | |
| 14437 | 14455 | Major (8) | 459918 | AAACAGCCGTTAGTTCCC | 5-9-5 | 39 | 166 |
| | | | | A | | | |
| 14439 | 14457 | Minor (10) | 459906 | AGAAACAGCTGTTAGTTC | 5-9-5 | 24 | 167 |
| | | | | C | | | |
| 14439 | 14457 | Major (10) | 459914 | AGAAACAGCCGTTAGTTC | 5-9-5 | 28 | 168 |
| | | | | C | | | |

TABLE 14
Comparison of inhibition of human HTT mRNA levels by ISIS 387916 and chimeric antisense
oligonucleotides targeted to SNP rs4690072 (nucleobases 62147 to 62173 of SEQ ID NO: 1)

| | | | | | | | SEQ |
|---|---|---|---|---|---|---|---|
| Start | Stop | Target | ISIS | | | % | ID |
| Site | Site | allele | No. | Sequence | Motif | inhibition | NO |
| 145466 | 145485 | n/a | 387916 | TCTCTATTGCACATTCCAAG | 5-10-5 | 98 | 6 |
| 62147 | 62165 | Major (6) | 460145 | GTGCTACCCAACCTTTCTG | 5-9-5 | 62 | 169 |
| 62148 | 62166 | Major (7) | 460144 | AGTGCTACCCAACCTTTCT | 5-9-5 | 61 | 170 |
| 62149 | 62167 | Major (8) | 460143 | CAGTGCTACCCAACCTTTC | 5-9-5 | 65 | 171 |
| 62150 | 62168 | Major (9) | 460142 | ACAGTGCTACCCAACCTTT | 5-9-5 | 83 | 172 |
| 62151 | 62169 | Major (10) | *435874 | CACAGTGCTACCCAACCTT | 5-9-5 | 76 | 28 |
| 62151 | 62169 | Major (10) | 460022 | CACAGTGCTACCCAACCTT | 4-10-5 | 75 | 28 |
| 62151 | 62169 | Major (10) | 460033 | CACAGTGCTACCCAACCTT | 4-11-4 | 89 | 28 |
| 62151 | 62168 | Major (9) | 460063 | ACAGTGCTACCCAACCTT | 4-9-5 | 77 | 173 |
| 62151 | 62169 | Major (10) | 460073 | CACAGTGCTACCCAACCTT | 5-10-4 | 86 | 28 |
| 62151 | 62169 | Major (10) | 460093 | CACAGTGCTACCCAACCTT | 5-8-6 | 61 | 28 |
| 62151 | 62169 | Major (10) | 460169 | CACAGTGCTACCCAACCTT | 6-7-6 | 16 | 28 |
| 62151 | 62169 | Major (10) | 460188 | CACAGTGCTACCCAACCTT | 6-8-5 | 53 | 28 |
| 62152 | 62168 | Major (9) | 459978 | ACAGTGCTACCCAACCT | 2-9-6 | 87 | 174 |
| 62152 | 62167 | Major (8) | 459999 | CAGTGCTACCCAACCT | 3-9-4 | 48 | 175 |
| 62152 | 62168 | Major (9) | 460012 | ACAGTGCTACCCAACCT | 3-9-5 | 84 | 174 |
| 62152 | 62168 | Major (9) | 460052 | ACAGTGCTACCCAACCT | 4-9-4 | 51 | 174 |
| 62152 | 62168 | Major (9) | 460083 | ACAGTGCTACCCAACCT | 5-7-5 | 37 | 174 |
| 62152 | 62168 | Major (9) | 460103 | ACAGTGCTACCCAACCT | 5-9-3 | 80 | 174 |
| 62152 | 62170 | Major (11) | 460137 | TCACAGTGCTACCCAACCT | 5-9-5 | 65 | 176 |
| 62152 | 62168 | Major (9) | 460179 | ACAGTGCTACCCAACCT | 6-9-2 | 67 | 174 |
| 62153 | 62167 | Major (8) | 459989 | CAGTGCTACCCAACC | 3-9-3 | 60 | 177 |
| 62153 | 62167 | Major (8) | 460043 | CAGTGCTACCCAACC | 4-7-4 | 24 | 177 |
| 62153 | 62171 | Major (12) | 460138 | ATCACAGTGCTACCCAACC | 5-9-5 | 76 | 178 |
| 62154 | 62172 | Major (13) | 460139 | TATCACAGTGCTACCCAAC | 5-9-5 | 68 | 179 |
| 62155 | 62173 | Major (14) | 460140 | ATATCACAGTGCTACCCAA | 5-9-5 | 79 | 180 |

TABLE 15
Comparison of inhibition of human HTT mRNA levels by ISIS 387916 and chimeric antisense
oligonucleotides targeted to SNP rs2298969 (nucleobases 125883 to 125911 of SEQ ID NO: 1)

| | | | | | | | SEQ |
|---|---|---|---|---|---|---|---|
| Start | Stop | Target | ISIS | | | % | ID |
| Site | Site | allele | No. | Sequence | Motif | inhibition | NO |
| 145466 | 145485 | n/a | 387916 | TCTCTATTGCACATTCCAAG | 5-10-5 | 98 | 6 |
| 125883 | 125901 | Minor (5) | 460166 | ATGCTGACTTGGGCCATTC | 5-9-5 | 83 | 181 |
| 125884 | 125902 | Minor (6) | 460165 | GATGCTGACTTGGGCCATT | 5-9-5 | 88 | 182 |
| 125885 | 125903 | Minor (7) | 460164 | GGATGCTGACTTGGGCCAT | 5-9-5 | 68 | 183 |
| 125886 | 125904 | Minor (8) | 460163 | GGGATGCTGACTTGGGCCA | 5-9-5 | 73 | 184 |
| 125887 | 125905 | Minor (9) | 460162 | AGGGATGCTGACTTGGGCC | 5-9-5 | 88 | 185 |
| 125888 | 125906 | Minor (10) | *435890 | AAGGGATGCTGACTTGGGC | 5-9-5 | 83 | 94 |
| 125888 | 125906 | Minor (10) | 460026 | AAGGGATGCTGACTTGGGC | 4-10-5 | 90 | 94 |
| 125888 | 125906 | Minor (10) | 460037 | AAGGGATGCTGACTTGGGC | 4-11-4 | 86 | 94 |
| 125888 | 125905 | Minor (9) | 460068 | AGGGATGCTGACTTGGGC | 4-9-5 | 90 | 186 |
| 125888 | 125906 | Minor (10) | 460076 | AAGGGATGCTGACTTGGGC | 5-10-4 | 90 | 94 |
| 125888 | 125906 | Minor (10) | 460096 | AAGGGATGCTGACTTGGGC | 5-8-6 | 88 | 94 |
| 125888 | 125906 | Minor (10) | 460171 | AAGGGATGCTGACTTGGGC | 6-7-6 | 87 | 94 |
| 125888 | 125906 | Minor (10) | 460190 | AAGGGATGCTGACTTGGGC | 6-8-5 | 69 | 94 |
| 125889 | 125905 | Minor (9) | 459983 | AGGGATGCTGACTTGGG | 2-9-6 | 80 | 187 |
| 125889 | 125904 | Minor (8) | 460005 | GGGATGCTGACTTGGG | 3-9-4 | 80 | 284 |
| 125889 | 125905 | Minor (9) | 460016 | AGGGATGCTGACTTGGG | 3-9-5 | 90 | 187 |
| 125889 | 125905 | Minor (9) | 460057 | AGGGATGCTGACTTGGG | 4-9-4 | 86 | 187 |
| 125889 | 125905 | Minor (9) | 460087 | AGGGATGCTGACTTGGG | 5-7-5 | 86 | 187 |
| 125889 | 125905 | Minor (9) | 460107 | AGGGATGCTGACTTGGG | 5-9-3 | 79 | 187 |
| 125889 | 125907 | Major (11) | 460157 | CAAGGGATGCTGACTTGGG | 5-9-5 | 88 | 188 |
| 125889 | 125905 | Minor (9) | 460181 | AGGGATGCTGACTTGGG | 6-9-2 | 62 | 187 |
| 125890 | 125904 | Minor (8) | 459972 | GGGATGCTGACTTGG | Uniform | 18 | 189 |
| 125890 | 125904 | Minor (8) | 459992 | GGGATGCTGACTTGG | 3-9-3 | 90 | 189 |
| 125890 | 125904 | Minor (8) | 460046 | GGGATGCTGACTTGG | 4-7-4 | 59 | 189 |
| 125890 | 125908 | Major (12) | 460158 | CCAAGGGATGCTGACTTGG | 5-9-5 | 79 | 190 |
| 125891 | 125909 | Major (13) | 460159 | GCCAAGGGATGCTGACTTG | 5-9-5 | 82 | 191 |
| 125892 | 125910 | Major (14) | 460160 | TGCCAAGGGATGCTGACTT | 5-9-5 | 87 | 192 |
| 125893 | 125911 | Major (15) | 460161 | CTGCCAAGGGATGCTGACT | 5-9-5 | 78 | 193 |

TABLE 16
Comparison of inhibition of human HTT mRNA levels by ISIS 387916 and chimeric antisense
oligonucleotides targeted to SNP rs7685686 (nucleobases 146781 to 146809 of SEQ ID NO: 1)

| | | | | | | | SEQ |
|---|---|---|---|---|---|---|---|
| Start | Stop | Target | ISIS | | | % | ID |
| Site | Site | allele | No. | Sequence | Motif | inhibition | NO |
| 145466 | 145485 | n/a | 387916 | TCTCTATTGCACATTCCAAG | 5-10-5 | 98 | 6 |
| 146781 | 146799 | Major (5) | 460156 | ATTGTCATCACCAGAAAAA | 5-9-5 | 88 | 194 |
| 146782 | 146800 | Major (6) | 460155 | AATTGTCATCACCAGAAAA | 5-9-5 | 89 | 195 |
| 146783 | 146801 | Major (7) | 460154 | AAATTGTCATCACCAGAAA | 5-9-5 | 89 | 196 |
| 146784 | 146802 | Major (8) | 460153 | TAAATTGTCATCACCAGAA | 5-9-5 | 93 | 197 |
| 146785 | 146803 | Major (9) | 460152 | ATAAATTGTCATCACCAGA | 5-9-5 | 95 | 198 |
| 146786 | 146804 | Major (10) | *435879 | AATAAATTGTCATCACCAG | 5-9-5 | 94 | 99 |
| 146786 | 146804 | Major (10) | 460024 | AATAAATTGTCATCACCAG | 4-10-5 | 88 | 99 |
| 146786 | 146804 | Major (10) | 460035 | AATAAATTGTCATCACCAG | 4-11-4 | 91 | 99 |
| 146786 | 146803 | Major (9) | 460065 | ATAAATTGTCATCACCAG | 4-9-5 | 96 | 199 |
| 146786 | 146804 | Major (10) | 460074 | AATAAATTGTCATCACCAG | 5-10-4 | 94 | 99 |
| 146786 | 146804 | Major (10) | 460095 | AATAAATTGTCATCACCAG | 5-8-6 | 92 | 99 |
| 146786 | 146804 | Major (10) | 460170 | AATAAATTGTCATCACCAG | 6-7-6 | 91 | 99 |
| 146786 | 146804 | Major (10) | 460189 | AATAAATTGTCATCACCAG | 6-8-5 | 94 | 99 |
| 146787 | 146803 | Major (9) | 459981 | ATAAATTGTCATCACCA | 2-9-6 | 85 | 200 |
| 146787 | 146802 | Major (8) | 460002 | TAAATTGTCATCACCA | 3-9-4 | 86 | 201 |
| 146787 | 146803 | Major (9) | 460014 | ATAAATTGTCATCACCA | 3-9-5 | 91 | 200 |
| 146787 | 146803 | Major (9) | 460055 | ATAAATTGTCATCACCA | 4-9-4 | 90 | 200 |
| 146787 | 146803 | Major (9) | 460085 | ATAAATTGTCATCACCA | 5-7-5 | 94 | 200 |
| 146787 | 146803 | Major (9) | 460104 | ATAAATTGTCATCACCA | 5-9-3 | 93 | 200 |
| 146787 | 146805 | Major (11) | 460147 | TAATAAATTGTCATCACCA | 5-9-5 | 91 | 202 |
| 146787 | 146803 | Major (9) | 460180 | ATAAATTGTCATCACCA | 6-9-2 | 91 | 200 |
| 146788 | 146802 | Major (8) | 459970 | TAAATTGTCATCACC | Uniform | 9 | 203 |
| 146788 | 146802 | Major (8) | 459990 | TAAATTGTCATCACC | 3-9-3 | 67 | 203 |
| 146788 | 146802 | Major (8) | 460045 | TAAATTGTCATCACC | 4-7-4 | 84 | 203 |
| 146788 | 146806 | Major (12) | 460148 | TTAATAAATTGTCATCACC | 5-9-5 | 88 | 204 |
| 146789 | 146807 | Major (13) | 460149 | ATTAATAAATTGTCATCAC | 5-9-5 | 32 | 205 |
| 146790 | 146808 | Major (14) | 460150 | TATTAATAAATTGTCATCA | 5-9-5 | 29 | 206 |
| 146791 | 146809 | Major (15) | 460151 | CTATTAATAAATTGTCATC | 5-9-5 | 33 | 207 |

TABLE 17
Comparison of inhibition of human HTT mRNA levels by ISIS 387916 and chimeric antisense
oligonucleotides targeted to SNP rs362331 (nucleobases 155474 to 155502 of SEQ ID NO: 1)

| | | | | | | | SEQ |
|---|---|---|---|---|---|---|---|
| Start | Stop | Target | ISIS | | | % | ID |
| Site | Site | allele | No. | Sequence | Motif | inhibition | NO |
| 145466 | 145485 | n/a | 387916 | TCTCTATTGCACATTCCAAG | 5-10-5 | 98 | 6 |
| 155474 | 155492 | Major (5) | 460136 | CAGTAGATGAGGGAGCAGG | 5-9-5 | 81 | 208 |
| 155475 | 155493 | Major (6) | 460135 | ACAGTAGATGAGGGAGCAG | 5-9-5 | 84 | 209 |
| 155476 | 155494 | Major (7) | 460134 | CACAGTAGATGAGGGAGCA | 5-9-5 | 87 | 210 |
| 155477 | 155495 | Major (8) | 460133 | ACACAGTAGATGAGGGAGC | 5-9-5 | 85 | 211 |
| 155478 | 155496 | Major (9) | 460132 | CACACAGTAGATGAGGGAG | 5-9-5 | 86 | 212 |
| 155479 | 155497 | Major (10) | *435870 | GCACACAGTAGATGAGGGA | 5-9-5 | 91 | 103 |
| 155479 | 155497 | Major (10) | 460019 | GCACACAGTAGATGAGGGA | 4-10-5 | 92 | 103 |
| 155479 | 155497 | Major (10) | 460031 | GCACACAGTAGATGAGGGA | 4-11-4 | 95 | 103 |
| 155479 | 155496 | Major (9) | 460061 | CACACAGTAGATGAGGGA | 4-9-5 | 87 | 213 |
| 155479 | 155497 | Major (10) | 460071 | GCACACAGTAGATGAGGGA | 5-10-4 | 94 | 103 |
| 155479 | 155497 | Major (10) | 460090 | GCACACAGTAGATGAGGGA | 5-8-6 | 86 | 103 |
| 155479 | 155497 | Major (10) | 460168 | GCACACAGTAGATGAGGGA | 6-7-6 | 84 | 103 |
| 155479 | 155497 | Major (10) | 460187 | GCACACAGTAGATGAGGGA | 6-8-5 | 89 | 103 |
| 155480 | 155496 | Major (9) | 459977 | CACACAGTAGATGAGGG | 2-9-6 | 90 | 214 |
| 155480 | 155495 | Major (8) | 459996 | ACACAGTAGATGAGGG | 3-9-4 | 37 | 215 |
| 155480 | 155496 | Major (9) | 460009 | CACACAGTAGATGAGGG | 3-9-5 | 90 | 214 |
| 155480 | 155496 | Major (9) | 460051 | CACACAGTAGATGAGGG | 4-9-4 | 73 | 214 |
| 155480 | 155496 | Major (9) | 460081 | CACACAGTAGATGAGGG | 5-7-5 | 77 | 214 |
| 155480 | 155496 | Major (9) | 460101 | CACACAGTAGATGAGGG | 5-9-3 | 84 | 214 |
| 155480 | 155498 | Major (11) | 460127 | TGCACACAGTAGATGAGGG | 5-9-5 | 89 | 216 |
| 155480 | 155496 | Major (9) | 460178 | CACACAGTAGATGAGGG | 6-9-2 | 92 | 214 |
| 155481 | 155495 | Major (8) | 459967 | ACACAGTAGATGAGG | Uniform | 81 | 217 |
| 155481 | 155495 | Major (8) | 459987 | ACACAGTAGATGAGG | 3-9-3 | 18 | 217 |
| 155481 | 155495 | Major (8) | 460041 | ACACAGTAGATGAGG | 4-7-4 | 54 | 217 |
| 155481 | 155499 | Major (12) | 460128 | GTGCACACAGTAGATGAGG | 5-9-5 | 73 | 218 |
| 155482 | 155500 | Major (13) | 460129 | AGTGCACACAGTAGATGAG | 5-9-5 | 86 | 219 |
| 155483 | 155501 | Major (14) | 460130 | AAGTGCACACAGTAGATGA | 5-9-5 | 60 | 220 |
| 155484 | 155502 | Major (15) | 460131 | GAAGTGCACACAGTAGATG | 5-9-5 | 73 | 221 |

TABLE 18
Comparison of inhibition of human HTT mRNA levels by ISIS 387916 and chimeric antisense
oligonucleotides targeted to SNP rs362306 (nucleobases 181739 to 181767 of SEQ ID NO: 1)

| | | | | | | | SEQ |
|---|---|---|---|---|---|---|---|
| Start | Stop | Target | ISIS | | | % | ID |
| Site | Site | allele | No. | Sequence | Motif | inhibition | NO |
| 145466 | 145485 | n/a | 387916 | TCTCTATTGCACATTCCAAG | 5-10-5 | 98 | 6 |
| 181739 | 181757 | Major (5) | 460126 | GCTGCAACCTGGCAACAAC | 5-9-5 | 87 | 222 |
| 181740 | 181758 | Major (6) | 460125 | AGCTGCAACCTGGCAACAA | 5-9-5 | 70 | 223 |
| 181741 | 181759 | Major (7) | 460123 | CAGCTGCAACCTGGCAACA | 5-9-5 | 83 | 224 |
| 181742 | 181760 | Major (8) | 460121 | GCAGCTGCAACCTGGCAAC | 5-9-5 | 47 | 225 |
| 181743 | 181761 | Major (9) | 460118 | AGCAGCTGCAACCTGGCAA | 5-9-5 | 75 | 226 |
| 181744 | 181762 | Major (10) | *435869 | GAGCAGCTGCAACCTGGCA | 5-9-5 | 91 | 149 |
| 181744 | 181762 | Major (10) | 460018 | GAGCAGCTGCAACCTGGCA | 4-10-5 | 86 | 149 |
| 181744 | 181762 | Major (10) | 460028 | GAGCAGCTGCAACCTGGCA | 4-11-4 | 89 | 149 |
| 181744 | 181761 | Major (9) | 460058 | AGCAGCTGCAACCTGGCA | 4-9-5 | 85 | 227 |
| 181744 | 181762 | Major (10) | 460069 | GAGCAGCTGCAACCTGGCA | 5-10-4 | 91 | 149 |
| 181744 | 181762 | Major (10) | 460089 | GAGCAGCTGCAACCTGGCA | 5-8-6 | 54 | 149 |
| 181744 | 181762 | Major (10) | 460167 | GAGCAGCTGCAACCTGGCA | 6-7-6 | 85 | 149 |
| 181744 | 181762 | Major (10) | 460186 | GAGCAGCTGCAACCTGGCA | 6-8-5 | 84 | 149 |
| 181745 | 181761 | Major (9) | 459975 | AGCAGCTGCAACCTGGC | 2-9-6 | 86 | 228 |
| 181745 | 181760 | Major (8) | 459995 | GCAGCTGCAACCTGGC | 3-9-4 | 87 | 229 |
| 181745 | 181761 | Major (9) | 460008 | AGCAGCTGCAACCTGGC | 3-9-5 | 83 | 228 |
| 181745 | 181761 | Major (9) | 460049 | AGCAGCTGCAACCTGGC | 4-9-4 | 88 | 228 |
| 181745 | 181761 | Major (9) | 460079 | AGCAGCTGCAACCTGGC | 5-7-5 | 46 | 228 |
| 181745 | 181761 | Major (9) | 460099 | AGCAGCTGCAACCTGGC | 5-9-3 | 44 | 228 |
| 181745 | 181763 | Major (11) | 460108 | AGAGCAGCTGCAACCTGGC | 5-9-5 | 50 | 230 |
| 181745 | 181761 | Major (9) | 460177 | AGCAGCTGCAACCTGGC | 6-9-2 | 67 | 228 |
| 181746 | 181760 | Major (8) | 459966 | GCAGCTGCAACCTGG | Uniform | 26 | 231 |
| 181746 | 181760 | Major (8) | 459985 | GCAGCTGCAACCTGG | 3-9-3 | 69 | 231 |
| 181746 | 181760 | Major (8) | 460039 | GCAGCTGCAACCTGG | 4-7-4 | 56 | 231 |
| 181746 | 181764 | Major (12) | 460110 | AAGAGCAGCTGCAACCTGG | 5-9-5 | 75 | 232 |
| 181747 | 181765 | Major (13) | 460113 | CAAGAGCAGCTGCAACCTG | 5-9-5 | 36 | 233 |
| 181748 | 181766 | Major (14) | 460115 | GCAAGAGCAGCTGCAACCT | 5-9-5 | 78 | 234 |
| 181749 | 181767 | Major (15) | 460117 | TGCAAGAGCAGCTGCAACC | 5-9-5 | 73 | 235 |

TABLE 19
Comparison of inhibition of human HTT mRNA levels by ISIS 387916 and chimeric antisense
oligonucleotides targeted to SNP rsl006798 (nucleobases 198015 to 198035 of SEQ ID NO: 1)

| | | | | | | | SEQ |
|---|---|---|---|---|---|---|---|
| Start | Stop | Target | ISIS | | | % | ID |
| Site | Site | allele | No. | Sequence | Motif | inhibition | NO |
| 145466 | 145485 | n/a | 387916 | TCTCTATTGCACATTCCAAG | 5-10-5 | 98 | 6 |
| 198015 | 198033 | Minor (8) | 459911 | ACCATGATATCTCCAGCAC | 5-9-5 | 33 | 236 |
| 198015 | 198033 | Minor (8) | 459919 | ACCATGACATCTCCAGCAC | 5-9-5 | 26 | 237 |
| 198017 | 198035 | Major (10) | 459907 | CCACCATGATATCTCCAGC | 5-9-5 | 32 | 238 |
| 198017 | 198035 | Minor (10) | 459915 | CCACCATGACATCTCCAGC | 5-9-5 | 51 | 239 |

### Example 6: Dose-Dependent Antisense Inhibition of Human Huntingtin mRNA Levels in Coriell Fibroblast Cell Lines

Gapmers from the studies described in Example 5 were selected and tested at various doses in GM04281, GM02171, and GM02173B cell lines. Each cell line was plated at a density of 25,000 cells per well and transfected using electroporation with 750 nM, 1,500 nM, 3,000 nM, 6,000 nM, and 12,000 nM concentrations of antisense oligonucleotide, as specified in Tables 20, 21, and 22. After a treatment period of approximately 16 hours, RNA was isolated from the cells and HTT mRNA levels were measured by quantitative real-time PCR. Human HTT primer probe set RTS2617 was used to measure mRNA levels. HTT mRNA levels were adjusted according to total RNA content, as measured by RIBOGREEN. Results are presented as percent inhibition of HTT mRNA, relative to untreated control cells. IC₅₀ values are also provided in Tables 20, 21, and 22.

TABLE 20
Dose-dependent antisense inhibition
of human HTT in GM04281 cells

| ISIS | 750 | 1,500 | 3,000 | 6,000 | 12,000 | IC₅₀ |
|---|---|---|---|---|---|---|
| No. | nM | nM | nM | nM | nM | (μM) |
| 387916 | 56 | 81 | 89 | 96 | 98 | 0.6 |
| 435869 | 38 | 49 | 66 | 86 | 91 | 1.4 |
| 435874 | 33 | 27 | 37 | 49 | 62 | 8.4 |
| 435879 | 42 | 55 | 73 | 86 | 96 | 1.1 |
| 435890 | 39 | 51 | 74 | 83 | 89 | 1.3 |
| 459978 | 29 | 33 | 51 | 69 | 86 | 2.5 |
| 459992 | 14 | 27 | 51 | 54 | 84 | 3.2 |
| 460012 | 15 | 24 | 54 | 70 | 81 | 3.1 |
| 460016 | 3 | 36 | 48 | 71 | 77 | 3.3 |
| 460019 | 54 | 59 | 74 | 87 | 94 | 0.7 |
| 460026 | 48 | 47 | 71 | 79 | 88 | 0.8 |
| 460028 | 39 | 38 | 73 | 77 | 87 | 1.4 |
| 460031 | 44 | 62 | 72 | 87 | 92 | 0.9 |
| 460033 | 11 | 38 | 52 | 64 | 87 | 3.0 |
| 460065 | 43 | 54 | 74 | 89 | 96 | 1.1 |
| 460068 | 47 | 28 | 63 | 76 | 90 | 2.6 |
| 460069 | 38 | 50 | 65 | 77 | 91 | 1.4 |
| 460071 | 53 | 61 | 80 | 89 | 93 | 0.6 |
| 460073 | 16 | 39 | 42 | 58 | 75 | 4.0 |
| 460076 | 26 | 47 | 54 | 70 | 86 | 2.1 |
| 460085 | 48 | 60 | 79 | 89 | 94 | 0.8 |
| 460140 | 6 | 24 | 44 | 44 | 64 | 6.6 |
| 460142 | 2 | 38 | 46 | 46 | 68 | 4.8 |
| 460152 | 35 | 61 | 76 | 92 | 94 | 1.2 |
| 460157 | 51 | 36 | 53 | 74 | 89 | 2.6 |
| 460162 | 64 | 41 | 71 | 76 | 85 | 2.1 |
| 460165 | 41 | 50 | 56 | 76 | 84 | 1.5 |

TABLE 21
Dose-dependent antisense inhibition
of human HTT in GM02171 cells

| ISIS | 750 | 1,500 | 3,000 | 6,000 | 12,000 | IC₅₀ |
|---|---|---|---|---|---|---|
| No. | nM | nM | nM | nM | nM | (μM) |
| 387916 | 53 | 66 | 88 | 96 | 98 | 0.7 |
| 435869 | 4 | 20 | 36 | 63 | 86 | 3.9 |
| 435870 | 25 | 39 | 48 | 62 | 83 | 2.8 |
| 435874 | 12 | 20 | 18 | 27 | 37 | >12.0 |
| 435879 | 10 | 7 | 11 | 42 | 51 | 10.6 |
| 435890 | 10 | 23 | 29 | 29 | 55 | 9.2 |
| 459978 | 15 | 7 | 6 | 29 | 52 | 12.7 |
| 459992 | 11 | 19 | 26 | 39 | 62 | 8.7 |
| 460012 | 3 | 3 | 10 | 19 | 41 | >12.0 |
| 460016 | 0 | 14 | 12 | 22 | 48 | >12.0 |
| 460019 | 27 | 21 | 41 | 60 | 73 | 4.4 |
| 460026 | 9 | 25 | 30 | 46 | 58 | 7.8 |
| 460028 | 24 | 8 | 32 | 54 | 77 | 5.3 |
| 460031 | 8 | 25 | 42 | 60 | 83 | 3.8 |
| 460033 | 11 | 25 | 30 | 40 | 75 | 4.1 |
| 460065 | 11 | 16 | 11 | 31 | 53 | 10.3 |
| 460068 | 15 | 13 | 39 | 44 | 53 | 8.8 |
| 460069 | 17 | 28 | 37 | 60 | 79 | 3.9 |
| 460071 | 16 | 36 | 58 | 70 | 88 | 2.6 |
| 460073 | 5 | 19 | 24 | 33 | 56 | 8.7 |
| 460076 | 19 | 29 | 44 | 54 | 83 | 3.3 |
| 460085 | 10 | 15 | 17 | 28 | 31 | >12.0 |
| 460140 | 8 | 22 | 22 | 28 | 47 | >12.0 |
| 460142 | 11 | 24 | 28 | 36 | 38 | >12.0 |
| 460152 | 14 | 21 | 8 | 25 | 44 | 22 |
| 460157 | 22 | 21 | 29 | 44 | 66 | 6.7 |
| 460162 | 24 | 55 | 52 | 62 | 82 | 2.8 |
| 460165 | 14 | 34 | 50 | 69 | 81 | 3.1 |

TABLE 22
Dose-dependent antisense inhibition
of human HTT in GM02173B cells

| ISIS | 750 | 1,500 | 3,000 | 6,000 | 12,000 | IC₅₀ |
|---|---|---|---|---|---|---|
| No. | nM | nM | nM | nM | nM | (μM) |
| 387916 | 37 | 63 | 86 | 88 | 98 | 1.0 |
| 435869 | 10 | 20 | 43 | 70 | 85 | 3.5 |
| 435870 | 24 | 24 | 56 | 72 | 87 | 2.3 |
| 435874 | 0 | 11 | 12 | 30 | 44 | >12.0 |
| 435879 | 4 | 17 | 43 | 64 | 74 | 4.3 |
| 435890 | 31 | 29 | 54 | 57 | 69 | 4.4 |
| 459978 | 7 | 13 | 17 | 35 | 64 | 8.4 |
| 459992 | 18 | 15 | 30 | 51 | 71 | 5.7 |
| 460012 | 0 | 10 | 24 | 37 | 72 | 7.1 |
| 460016 | 15 | 5 | 30 | 38 | 59 | 9.5 |
| 460019 | 10 | 32 | 51 | 65 | 87 | 3.1 |
| 460026 | 0 | 34 | 21 | 55 | 65 | 6.4 |
| 460028 | 0 | 14 | 31 | 51 | 77 | 5.2 |
| 460031 | 0 | 31 | 53 | 71 | 88 | 3.2 |
| 460033 | 11 | 8 | 6 | 52 | 84 | 5.0 |
| 460065 | 19 | 37 | 53 | 58 | 74 | 3.6 |
| 460068 | 17 | 11 | 31 | 59 | 69 | 5.5 |
| 460069 | 11 | 21 | 37 | 55 | 75 | 4.6 |
| 460071 | 6 | 42 | 61 | 83 | 88 | 2.6 |
| 460073 | 7 | 13 | 19 | 49 | 66 | 6.3 |
| 460076 | 27 | 31 | 49 | 43 | 81 | 2.9 |
| 460085 | 17 | 34 | 51 | 54 | 68 | 4.4 |
| 460140 | 0 | 2 | 28 | 18 | 46 | >12.0 |
| 460142 | 2 | 32 | 37 | 42 | 59 | 7.6 |
| 460152 | 17 | 32 | 35 | 51 | 66 | 5.5 |
| 460157 | 9 | 34 | 38 | 52 | 74 | 4.5 |
| 460162 | 22 | 45 | 57 | 65 | 79 | 2.5 |
| 460165 | 5 | 45 | 52 | 72 | 84 | 3.2 |

### Example 7: Antisense Inhibition of Human HTT in GM04281 Cells and GM02171 Cells

Additional antisense oligonucleotides were designed based on the gapmers selected from studies described in Example 2. These oligonucleotides were designed by creating gapmers shifted slightly upstream and downstream (i.e. “microwalk”) of the original gapmers from Table 4.

The gapmers were tested in the GM04281 and the GM02171 cell lines. Cultured GM04281 or GM02171 cells at a density of 25,000 cells per well were transfected using electroporation with 10,000 nM antisense oligonucleotide. After a treatment period of approximately 24 hours, RNA was isolated from the cells and HTT mRNA levels were measured by quantitative real-time PCR using primer probe set RTS2617. HTT mRNA levels were adjusted according to total RNA content, as measured by RIBOGREEN. Results are presented as percent inhibition of HTT mRNA, relative to untreated control cells.

The gapmers, from which the newly designed oligonucleotides were derived, were also included in the assay. These parent gapmers, ISIS 435294, ISIS 435295, ISIS 435301, ISIS 435303, ISIS 435304, ISIS 435305, ISIS 435308, ISIS 435330, ISIS 435331, ISIS 435337, ISIS 435339, ISIS 435340, ISIS 435341, ISIS 435344, ISIS 435862, ISIS 435863, ISIS 435864, ISIS 435866, ISIS 435867, ISIS 435868, ISIS 435871, ISIS 435873, ISIS 435875, ISIS 435876, ISIS 435878, ISIS 435880, ISIS 435881, ISIS 435882, ISIS 435884, ISIS 435890, and ISIS 435897 are marked with an asterisk (*) in the table. ISIS 387916 was included in the study as a benchmark oligonucleotide against which the potency of the antisense oligonucleotides targeting nucleotides overlapping each SNP position could be compared.

The chimeric antisense oligonucleotides in Tables 23-48 were designed as 5-9-5 MOE gapmers. The 5-9-5 gapmers are 19 nucleotides in length, wherein the central gap segment is comprised of nine 2′-deoxynucleotides and is flanked on both 5′ and 3′ directions by wings comprising 5 nucleotides each. Each nucleotide in the 5′ wing segment and each nucleotide in the 3′ wing segment has a 2′-MOE modification. The internucleoside linkages throughout each gapmer are phosphorothioate (P═S) linkages. All cytosine nucleobases throughout each gapmer are 5-methylcytosines.

The gapmers are organized in Tables 23-48, according to the SNP site they target. “Start site” indicates the 5′-most nucleotide to which the gapmer is targeted. “Stop site” indicates the 3′-most nucleotide to which the gapmer is targeted. ‘Target allele’ indicates whether the gapmer is targeted to the major or the minor allele. The number in parentheses indicates the position on the oligonucleotide opposite to the SNP position.

TABLE 23
Comparison of inhibition of human HTT mRNA levels by ISIS 387916 and chimeric antisense
oligonucleotides targeted to SNP rs3856973 (nucleobases 19815 to 19835 of SEQ ID NO: 1)

| | | | | | % | % | |
|---|---|---|---|---|---|---|---|
| | | | | | inhibition | inhibition | |
| Start | Stop | ISIS | Target | | in | in | SEQ |
| Site | Site | No | allele | Sequence | GM04281 | GM02171 | ID NO |
| 145466 | 145485 | 387916 | n/a | TCTCTATTGCACATTCCAAG | 100 | 99 | 6 |
| 19815 | 19833 | *435330 | Major (8) | TAACACTCGATTAACCCTG | 88 | 31 | 8 |
| 19816 | 19834 | 476441 | Major (9) | TTAACACTCGATTAACCCT | 88 | 0 | 240 |
| 19817 | 19835 | *435294 | Major (10) | GTTAACACTCGATTAACCC | 72 | 30 | 10 |

TABLE 24
Comparison of inhibition of human HTT mRNA levels by ISIS 387916 and chimeric antisense
oligonucleotides targeted to SNP rs2285086 (nucleobases 28901 to 28921 of SEQ ID NO: 1)

| | | | | | % | % | |
|---|---|---|---|---|---|---|---|
| | | | | | inhibition | inhibition | |
| Start | Stop | ISIS | Target | | in | in | SEQ |
| Site | Site | No | allele | Sequence | GM04281 | GM02171 | ID NO |
| 145466 | 145485 | 387916 | n/a | TCTCTATTGCACATTCCAAG | 100 | 99 | 6 |
| 28901 | 28919 | 463570 | Major (8) | TAGTTCATCCCAGTGAGAA | 66 | 12 | 241 |
| 28902 | 28920 | 463573 | Major (9) | CTAGTTCATCCCAGTGAGA | 66 | 36 | 242 |
| 28903 | 28921 | *435864 | Major (10) | GCTAGTTCATCCCAGTGAG | 40 | 18 | 12 |

TABLE 25
Comparison of inhibition of human HTT mRNA levels by ISIS 387916 and chimeric antisense
oligonucleotides targeted to SNP rs7659144 (nucleobases 37963 to 37983 of SEQ ID NO: 1)

| | | | | | % | % | |
|---|---|---|---|---|---|---|---|
| | | | | | inhibition | inhibition | SEQ |
| Start | Stop | ISIS | Target | | in | in | ID |
| Site | Site | No | allele | Sequence | GM04281 | GM02171 | NO |
| 145466 | 145485 | 387916 | n/a | TCTCTATTGCACATTCCAAG | 100 | 99 | 6 |
| 37963 | 37981 | 476462 | Major (8) | GAAATGGGTTTTTCCACAT | 38 | 0 | 243 |
| 37964 | 37982 | 476439 | Major (9) | GGAAATGGGTTTTTCCACA | 80 | 45 | 244 |
| 37965 | 37983 | *435878 | Major (10) | TGGAAATGGGTTTTTCCAC | 76 | 3 | 14 |

TABLE 26
Comparison of inhibition of human HTT mRNA levels by ISIS 387916 and chimeric antisense
oligonucleotides targeted to SNP rs16843804 (nucleobases 44032 to 44052 of SEQ ID NO: 1)

| | | | | | % | % | |
|---|---|---|---|---|---|---|---|
| | | | | | inhibition | inhibition | SEQ |
| Start | Stop | ISIS | Target | | in | in | ID |
| Site | Site | No | allele | Sequence | GM04281 | GM02171 | NO |
| 145466 | 145485 | 387916 | n/a | TCTCTATTGCACATTCCAAG | 100 | 99 | 6 |
| 44032 | 44050 | 476471 | Major (8) | TAACCGTGGCATGGGCAGT | 82 | 53 | 245 |
| 44033 | 44051 | 476452 | Major (9) | TTAACCGTGGCATGGGCAG | 84 | 44 | 246 |
| 44034 | 44052 | *435863 | Major (10) | TTTAACCGTGGCATGGGCA | 89 | 89 | 16 |

TABLE 27
Comparison of inhibition of human HTT mRNA levels by ISIS 387916 and chimeric antisense
oligonucleotides targeted to SNP rs2024115 (nucleobases 44210 to 44230 of SEQ ID NO: 1)

| | | | | | % | % | |
|---|---|---|---|---|---|---|---|
| | | | | | inhibition | inhibition | SEQ |
| Start | Stop | | Target | | in | in | ID |
| Site | Site | ISIS No | allele | Sequence | GM04281 | GM02171 | NO |
| 145466 | 145485 | 387916 | n/a | TCTCTATTGCACATTCCAAG | 100 | 99 | 6 |
| 44210 | 44228 | *435331 | Major (8) | TTCAAGCTAGTAACGATGC | 84 | 20 | 18 |
| 44211 | 44229 | 476447 | Major (9) | CTTCAAGCTAGTAACGATG | 87 | 57 | 247 |
| 44212 | 44230 | *435295 | Major (10) | ACTTCAAGCTAGTAACGAT | 85 | 67 | 20 |

TABLE 28
Comparison of inhibition of human HTT mRNA levels by ISIS 387916 and chimeric antisense
oligonucleotides targeted to SNP rs10015979 (nucleobases 49084 to 49104 of SEQ ID NO: 1)

| | | | | | % | % | |
|---|---|---|---|---|---|---|---|
| | | | | | inhibition | inhibition | SEQ |
| Start | Stop | | Target | | in | in | ID |
| Site | Site | ISIS No | allele | Sequence | GM04281 | GM02171 | NO |
| 145466 | 145485 | 387916 | n/a | TCTCTATTGCACATTCCAAG | 100 | 99 | 6 |
| 49084 | 49102 | 476470 | Major (8) | AGCTAGGTTAAAGAGTCAC | 55 | 74 | 248 |
| 49085 | 49103 | 476450 | Major (9) | CAGCTAGGTTAAAGAGTCA | 44 | 5 | 249 |
| 49086 | 49104 | *435862 | Major (10) | GCAGCTAGGTTAAAGAGTC | 56 | 49 | 22 |

TABLE 29
Comparison of inhibition of human HTT mRNA levels by ISIS 387916 and chimeric antisense
oligonucleotides targeted to SNP rs7691627 (nucleobases 51052 to 51072 of SEQ ID NO: 1)

| | | | | | % | % | |
|---|---|---|---|---|---|---|---|
| | | | | | inhibition | inhibition | SEQ |
| Start | Stop | ISIS | Target | | in | in | ID |
| Site | Site | No | allele | Sequence | GM04281 | GM02171 | NO |
| 145466 | 145485 | 387916 | n/a | TCTCTATTGCACATTCCAAG | 100 | 99 | 6 |
| 51052 | 51070 | 476467 | Major (8) | TAAGAAACACAATCAAAGA | 45 | 21 | 250 |
| 51053 | 51071 | 476445 | Major (9) | ATAAGAAACACAATCAAAG | 34 | 1 | 251 |
| 51054 | 51072 | *435880 | Major (10) | AATAAGAAACACAATCAAA | 68 | 7 | 24 |

TABLE 30
Comparison of inhibition of human HTT mRNA levels by ISIS 387916 and chimeric antisense
oligonucleotides targeted to SNP rs6446723 (nucleobases 66455 to 66475 of SEQ ID NO: 1)

| | | | | | % | % | |
|---|---|---|---|---|---|---|---|
| | | | | | inhibition | inhibition | |
| Start | Stop | | Target | | in | in | SEQ |
| Site | Site | ISIS No | allele | Sequence | GM04281 | GM02171 | ID NO |
| 145466 | 145485 | 387916 | n/a | TCTCTATTGCACATTCCAAG | 100 | 99 | 6 |
| 66455 | 66473 | 476463 | Major (8) | ATTTTCTAGACTTTATGAT | 37 | 7 | 252 |
| 66456 | 66474 | 476440 | Major (9) | AATTTTCTAGACTTTATGA | 57 | 0 | 253 |
| 66457 | 66475 | *435875 | Major (10) | TAATTTTCTAGACTTTATG | 42 | 0 | 30 |

TABLE 31
Comparison of inhibition of human HTT mRNA levels by ISIS 387916 and a chimeric antisense
oligonucleotide targeted to SNP rs363064 (nucleobases 81053 to 81071 of SEQ ID NO: 1)

| | | | | | % | % | |
|---|---|---|---|---|---|---|---|
| | | | | | inhibition | inhibition | SEQ |
| Start | Stop | | Target | | in | in | ID |
| Site | Site | ISIS No | allele | Sequence | GM04281 | GM02171 | NO |
| 145466 | 145485 | 387916 | n/a | TCTCTATTGCACATTCCAAG | 100 | 99 | 6 |
| 81053 | 81071 | 476461 | Major (9) | GAGAATACGGGTAACATTT | 87 | 62 | 254 |

TABLE 32
Comparison of inhibition of human HTT mRNA levels by ISIS 387916 and chimeric antisense
oligonucleotides targeted to SNP rs11731237 (nucleobases 91455 to 91475 of SEQ ID NO: 1)

| | | | | | % | % | |
|---|---|---|---|---|---|---|---|
| | | | | | inhibition | inhibition | |
| Start | Stop | | Target | | in | in | SEQ |
| Site | Site | ISIS No | allele | Sequence | GM04281 | GM02171 | ID NO |
| 145466 | 145485 | 387916 | n/a | TCTCTATTGCACATTCCAAG | 100 | 99 | 6 |
| 91455 | 91473 | 476468 | Major (8) | TGGGCAGGAAGGACTGAAC | 58 | 56 | 255 |
| 91456 | 91474 | 476448 | Major (9) | GTGGGCAGGAAGGACTGAA | 61 | 69 | 256 |
| 91457 | 91475 | *435884 | Major (10) | GGTGGGCAGGAAGGACTGA | 59 | 49 | 68 |

TABLE 33
Comparison of inhibition of human HTT mRNA levels by ISIS 387916 and chimeric antisense
oligonucleotides targeted to SNP rs4690073 (nucleobases 99792 to 99812 of SEQ ID NO: 1)

| | | | | | % | % | |
|---|---|---|---|---|---|---|---|
| | | | | | inhibition | inhibition | |
| Start | Stop | | Target | | in | in | SEQ |
| Site | Site | ISIS No | allele | Sequence | GM04281 | GM02171 | ID NO |
| 145466 | 145485 | 387916 | n/a | TCTCTATTGCACATTCCAAG | 100 | 99 | 6 |
| 99792 | 99810 | *435337 | Major (8) | CCTAAATCAATCTACAAGT | 69 | 7 | 70 |
| 99793 | 99811 | 476446 | Major (9) | CCCTAAATCAATCTACAAG | 61 | 0 | 257 |
| 99794 | 99812 | *435301 | Major (10) | TCCCTAAATCAATCTACAA | 63 | 1 | 72 |

TABLE 34
Comparison of inhibition of human HTT mRNA levels by ISIS 387916 and chimeric antisense
oligonucleotides targeted to SNP rs34315806 (nucleobases 101676 to 101696 of SEQ ID NO: 1)

| | | | | | % | % | |
|---|---|---|---|---|---|---|---|
| | | | | | inhibition | inhibition | |
| Start | Stop | | Target | | in | in | SEQ |
| Site | Site | ISIS No | allele | Sequence | GM04281 | GM02171 | ID NO |
| 145466 | 145485 | 387916 | n/a | TCTCTATTGCACATTCCAAG | 100 | 99 | 6 |
| 101676 | 101694 | 463569 | Major (8) | CTTTTCCGTGCTGTTCTGA | 96 | 95 | 258 |
| 101677 | 101695 | 463572 | Major (9) | ACTTTTCCGTGCTGTTCTG | 93 | 91 | 259 |
| 101678 | 101696 | 463567 | Major (10) | AACTTTTCCGTGCTGTTCT | 98 | 97 | 260 |

TABLE 35
Comparison of inhibition of human HTT mRNA levels by ISIS 387916 and chimeric antisense
oligonucleotides targeted to SNP rs363099 (nucleobases 101698 to 101718 of SEQ ID NO: 1)

| | | | | | % | % | |
|---|---|---|---|---|---|---|---|
| | | | | | inhibition | inhibition | |
| Start | Stop | | Target | | in | in | SEQ |
| Site | Site | ISIS No | allele | Sequence | GM04281 | GM02171 | ID NO |
| 145466 | 145485 | 387916 | n/a | TCTCTATTGCACATTCCAAG | 100 | 99 | 6 |
| 101698 | 101716 | *435339 | Major (8) | CTGAGCGGAGAAACCCTCC | 94 | 85 | 80 |
| 101699 | 101717 | 476458 | Major (9) | GCTGAGCGGAGAAACCCTC | 92 | 79 | 261 |
| 101700 | 101718 | *435303 | Major (10) | GGCTGAGCGGAGAAACCCT | 96 | 93 | 82 |

TABLE 36
Comparison of inhibition of human HTT mRNA levels by ISIS 387916 and chimeric antisense
oligonucleotides targeted to SNP rs363096 (nucleobases 119663 to 119683 of SEQ ID NO: 1)

| | | | | | % | % | |
|---|---|---|---|---|---|---|---|
| | | | | | inhibition | inhibition | |
| Start | Stop | | Target | | in | in | SEQ |
| Site | Site | ISIS No | allele | Sequence | GM04281 | GM02171 | ID NO |
| 145466 | 145485 | 387916 | n/a | TCTCTATTGCACATTCCAAG | 100 | 99 | 6 |
| 119663 | 119681 | *435340 | Major (8) | TTCCCTAAAAACAAAAACA | 42 | 21 | 85 |
| 119664 | 119682 | 476451 | Major (9) | ATTCCCTAAAAACAAAAAC | 0 | 0 | 262 |
| 119665 | 119683 | *435304 | Major (10) | GATTCCCTAAAAACAAAAA | 41 | 27 | 87 |

TABLE 37
Comparison of inhibition of human HTT mRNA levels by ISIS 387916 and chimeric antisense
oligonucleotides targeted to SNP rs2298967 (nucleobases 125389 to 125409 of SEQ ID NO: 1)

| | | | | | % | % | |
|---|---|---|---|---|---|---|---|
| | | | | | inhibition | inhibition | SEQ |
| Start | Stop | | Target | | in | in | ID |
| Site | Site | ISIS No | allele | Sequence | GM04281 | GM02171 | NO |
| 145466 | 145485 | 387916 | n/a | TCTCTATTGCACATTCCAAG | 100 | 99 | 6 |
| 125389 | 125407 | *435341 | Major (8) | CTTTTCTATTGTCTGTCCC | 83 | 65 | 89 |
| 125390 | 125408 | 476459 | Major (9) | GCTTTTCTATTGTCTGTCC | 89 | 82 | 263 |
| 125391 | 125409 | *435305 | Major (10) | TGCTTTTCTATTGTCTGTC | 92 | 85 | 91 |

TABLE 38
Comparison of inhibition of human HTT mRNA levels by ISIS 387916 and a chimeric antisense
oligonucleotide targeted to SNP rs2298969 (nucleobases 125888 to 125906 of SEQ ID NO: 1)

| | | | | | % | % | |
|---|---|---|---|---|---|---|---|
| | | | | | inhibition | inhibition | SEQ |
| Start | Stop | | Target | | in | in | ID |
| Site | Site | ISIS No | allele | Sequence | GM04281 | GM02171 | NO |
| 145466 | 145485 | 387916 | n/a | TCTCTATTGCACATTCCAAG | 100 | 99 | 6 |
| 125888 | 125906 | *435890 | Minor (10) | AAGGGATGCTGACTTGGGC | 91 | 64 | 94 |

TABLE 39
Comparison of inhibition of human HTT mRNA levels by ISIS 387916 and chimeric antisense
oligonucleotides targeted to SNP rs6844859 (nucleobases 130128 to 130148 of SEQ ID NO: 1)

| | | | | | % | % | |
|---|---|---|---|---|---|---|---|
| | | | | | inhibition | inhibition | SEQ |
| Start | Stop | | Target | | in | in | ID |
| Site | Site | ISIS No | allele | Sequence | GM04281 | GM02171 | NO |
| 145466 | 145485 | 387916 | n/a | TCTCTATTGCACATTCCAAG | 100 | 99 | 6 |
| 130128 | 130146 | 476466 | Major (8) | CTTCCTCACTGAGGATGAA | 87 | 64 | 264 |
| 130129 | 130147 | 476444 | Major (9) | CCTTCCTCACTGAGGATGA | 92 | 77 | 265 |
| 130130 | 130148 | *435876 | Major (10) | ACCTTCCTCACTGAGGATG | 94 | 87 | 95 |

TABLE 40
Comparison of inhibition of human HTT mRNA levels by ISIS 387916 and chimeric antisense
oligonucleotides targeted to SNP rs363092 (nucleobases 135671 to 135691 of SEQ ID NO: 1)

| | | | | | % | % | |
|---|---|---|---|---|---|---|---|
| | | | | | inhibition | inhibition | SEQ |
| Start | Stop | | Target | | in | in | ID |
| Site | Site | ISIS No | allele | Sequence | GM04281 | GM02171 | NO |
| 145466 | 145485 | 387916 | n/a | TCTCTATTGCACATTCCAAG | 100 | 99 | 6 |
| 135671 | 135689 | 476464 | Major (8) | AACCACTTTGGGATGAATA | 51 | 71 | 266 |
| 135672 | 135690 | 476442 | Major (9) | AAACCACTTTGGGATGAAT | 58 | 59 | 267 |
| 135673 | 135691 | *435897 | Minor (10) | CAAACCACTTTGGGATGAA | 48 | 78 | 98 |

TABLE 41
Comparison of inhibition of human HTT mRNA levels by ISIS 387916 and chimeric antisense
oligonucleotides targeted to SNP rs363088 (nucleobases 149972 to 149992 of SEQ ID NO: 1)

| | | | | | % | % | |
|---|---|---|---|---|---|---|---|
| | | | | | inhibition | inhibition | SEQ |
| Start | Stop | | Target | | in | in | ID |
| Site | Site | ISIS No | allele | Sequence | GM04281 | GM02171 | NO |
| 145466 | 145485 | 387916 | n/a | TCTCTATTGCACATTCCAAG | 100 | 99 | 6 |
| 149972 | 149990 | 476476 | Major (8) | ACAGCTATCTTCTCATCAA | 90 | 65 | 268 |
| 149973 | 149991 | 476460 | Major (9) | CACAGCTATCTTCTCATCA | 86 | 39 | 269 |
| 149974 | 149992 | *435871 | Major (10) | TCACAGCTATCTTCTCATC | 91 | 54 | 101 |

TABLE 42
Comparison of inhibition of human HTT mRNA levels by
ISIS 387916 and chimeric antisense oligonucleotides targeted to
SNP rs916171 (nucleobases 156457 to 156477 of SEQ ID NO: 1)

| | | | | | % | % | SEQ |
|---|---|---|---|---|---|---|---|
| Start | Stop | ISIS | Target | | inhibition | inhibition | ID |
| Site | Site | No | allele | Sequence | in GM04281 | in GM02171 | NO |
| 145466 | 145485 | 387916 | n/a | TCTCTATTGC | 100 | 99 | 6 |
| | | | | ACATTCCAAG | | | |
| 156457 | 156475 | 476465 | Major (8) | GAACAAAGAG | 38 | 0 | 270 |
| | | | | AAGAATTTC | | | |
| 156458 | 156476 | 476443 | Major (9) | AGAACAAAGA | 58 | 0 | 271 |
| | | | | GAAGAATTT | | | |
| 156459 | 156477 | *435881 | Major (10) | CAGAACAAAG | 59 | 16 | 105 |
| | | | | AGAAGAATT | | | |

TABLE 43
Comparison of inhibition of human HTT mRNA levels by
ISIS 387916 and chimeric antisense oligonucleotides targeted to
SNP rs362275 (nucleobases 164244 to 164264 of SEQ ID NO: 1)

| | | | | | % | % | SEQ |
|---|---|---|---|---|---|---|---|
| Start | Stop | ISIS | Target | | inhibition | inhibition | ID |
| Site | Site | No | allele | Sequence | in GM04281 | in GM02171 | NO |
| 145466 | 145485 | 387916 | n/a | TCTCTATTGC | 100 | 99 | 6 |
| | | | | ACATTCCAAG | | | |
| 164244 | 164262 | 476473 | Major (8) | GAAGCCTGAT | 83 | 51 | 272 |
| | | | | AAAATCTCT | | | |
| 164245 | 164263 | 476454 | Major (9) | AGAAGCCTGA | 79 | 61 | 273 |
| | | | | TAAAATCTC | | | |
| 164246 | 164264 | *435868 | Major (10) | AAGAAGCCTG | 69 | 56 | 111 |
| | | | | ATAAAATCT | | | |

TABLE 44
Comparison of inhibition of human HTT mRNA levels by
ISIS 387916 and chimeric antisense oligonucleotides targeted to
SNP rs362273 (nucleobases 167061 to 167081 of SEQ ID NO: 1)

| | | | | | % | % | SEQ |
|---|---|---|---|---|---|---|---|
| Start | Stop | ISIS | Target | | inhibition | inhibition | ID |
| Site | Site | No | allele | Sequence | in GM04281 | in GM02171 | NO |
| 145466 | 145485 | 387916 | n/a | TCTCTATTGC | 100 | 99 | 6 |
| | | | | ACATTCCAAG | | | |
| 167061 | 167079 | 463568 | Major (8) | TGATCTGTAG | 96 | 78 | 274 |
| | | | | CAGCAGCTT | | | |
| 167062 | 167080 | 463571 | Major (9) | TTGATCTGTA | 95 | 86 | 275 |
| | | | | GCAGCAGCT | | | |
| 167063 | 167081 | 463566 | Major (10) | GTTGATCTGT | 94 | 78 | 276 |
| | | | | AGCAGCAGC | | | |

TABLE 45
Comparison of inhibition of human HTT mRNA levels by
ISIS 387916 and chimeric antisense oligonucleotides targeted to
SNP rs362272 (nucleobases 174622 to 174642 of SEQ ID NO: 1)

| | | | | | % | % | SEQ |
|---|---|---|---|---|---|---|---|
| Start | Stop | ISIS | Target | | inhibition | inhibition | ID |
| Site | Site | No | allele | Sequence | in GM04281 | in GM02171 | NO |
| 145466 | 145485 | 387916 | n/a | TCTCTATTGC | 100 | 99 | 6 |
| | | | | ACATTCCAAG | | | |
| 174622 | 174640 | *435344 | Major (8) | TAGAGGACGC | 78 | 63 | 120 |
| | | | | CGTGCAGGG | | | |
| 174623 | 174641 | 476456 | Major (9) | ATAGAGGACG | 87 | 60 | 277 |
| | | | | CCGTGCAGG | | | |
| 174624 | 174642 | *435308 | Major (10) | CATAGAGGAC | 76 | 48 | 122 |
| | | | | GCCGTGCAG | | | |

TABLE 46
Comparison of inhibition of human HTT mRNA levels by
ISIS 387916 and chimeric antisense oligonucleotides targeted to
SNP rs362271 (nucleobases 175160 to 175180 of SEQ ID NO: 1)

| | | | | | % | % | SEQ |
|---|---|---|---|---|---|---|---|
| Start | Stop | ISIS | Target | | inhibition | inhibition | ID |
| Site | Site | No | allele | Sequence | in GM04281 | in GM02171 | NO |
| 145466 | 145485 | 387916 | n/a | TCTCTATTGC | 100 | 99 | 6 |
| | | | | ACATTCCAAG | | | |
| 175160 | 175178 | 476472 | Major (8) | GTGTGTACAG | 85 | 52 | 278 |
| | | | | AACCTGCCG | | | |
| 175161 | 175179 | 476453 | Major (9) | CGTGTGTACA | 88 | 69 | 279 |
| | | | | GAACCTGCC | | | |
| 175162 | 175180 | *435867 | Major (10) | ACGTGTGTAC | 91 | 80 | 125 |
| | | | | AGAACCTGC | | | |

TABLE 47
Comparison of inhibition of human HTT mRNA levels by
ISIS 387916 and chimeric antisense oligonucleotides targeted to
SNP rs3775061 (nucleobases 178396 to 178416 of SEQ ID NO: 1)

| | | | | | % | % | SEQ |
|---|---|---|---|---|---|---|---|
| Start | Stop | ISIS | Target | | inhibition | inhibition | ID |
| Site | Site | No | allele | Sequence | in GM04281 | in GM02171 | NO |
| 145466 | 145485 | 387916 | n/a | TCTCTATTGC | 100 | 99 | 6 |
| | | | | ACATTCCAAG | | | |
| 178396 | 178414 | 476475 | Major (8) | TTCAGAATGC | 61 | 1 | 280 |
| | | | | CTCATCTGG | | | |
| 178397 | 178415 | 476457 | Major (9) | GTTCAGAATG | 80 | 50 | 281 |
| | | | | CCTCATCTG | | | |
| 178398 | 178416 | *435873 | Major (10) | TGTTCAGAAT | 80 | 43 | 127 |
| | | | | GCCTCATCT | | | |

TABLE 48
Comparison of inhibition of human HTT mRNA levels by
ISIS 387916 and chimeric antisense oligonucleotides targeted to
SNP rs362296 (nucleobases 186649 to 1786669 of SEQ ID NO: 1)

| | | | | | % | % | SEQ |
|---|---|---|---|---|---|---|---|
| Start | Stop | ISIS | Target | | inhibition | inhibition | ID |
| Site | Site | No | allele | Sequence | in GM04281 | in GM02171 | NO |
| 145466 | 145485 | 387916 | n/a | TCTCTATTGC | 100 | 99 | 6 |
| | | | | ACATTCCAAG | | | |
| 186649 | 186667 | 476469 | Major (8) | GGACAGGGTG | 80 | 58 | 282 |
| | | | | TGCTCTCCG | | | |
| 186650 | 186668 | 476449 | Major (9) | GGGACAGGGT | 80 | 64 | 283 |
| | | | | GTGCTCTCC | | | |
| 186651 | 186669 | *435882 | Major (10) | GGGGACAGGG | 61 | 61 | 155 |
| | | | | TGTGCTCTC | | | |

### Example 8: Dose-Dependent Antisense Inhibition of Human Huntingtin mRNA Levels in Coriell Fibroblast Cell Lines

Gapmers from the studies described in Example 7 were selected and tested at various doses in GM04281, GM02171, and GM02173B cell lines. Each cell line was plated at a density of 25,000 cells per well and transfected using electroporation with 750 nM, 1,500 nM, 3,000 nM, 6,000 nM, and 12,000 nM concentrations of antisense oligonucleotide, as specified in Tables 49, 50, and 51. After a treatment period of approximately 16 hours, RNA was isolated from the cells and HTT mRNA levels were measured by quantitative real-time PCR. Human HTT primer probe set RTS2617 was used to measure mRNA levels. HTT mRNA levels were adjusted according to total RNA content, as measured by RIBOGREEN. Results are presented as percent inhibition of HTT mRNA, relative to untreated control cells. IC₅₀ values are also provided in Tables 49, 50, and 51.

TABLE 49
Dose-dependent antisense inhibition
of human HTT in GM04281 cells

| ISIS | 750 | 1500 | 3000 | 6000 | 12000 | IC₅₀ |
|---|---|---|---|---|---|---|
| No. | nM | nM | nM | nM | nM | (μM) |
| 387916 | 67 | 88 | 95 | 97 | 99 | <0.8 |
| 463566 | 25 | 65 | 79 | 88 | 95 | 1.5 |
| 463567 | 34 | 73 | 90 | 93 | 98 | 1.1 |
| 463568 | 33 | 56 | 75 | 87 | 92 | 1.3 |
| 463571 | 32 | 21 | 70 | 90 | 93 | 1.4 |
| 476441 | 11 | 27 | 50 | 70 | 87 | 3.1 |
| 476444 | 20 | 31 | 68 | 49 | 93 | 2.3 |
| 476449 | 4 | 28 | 34 | 47 | 77 | 4.9 |
| 476453 | 21 | 21 | 48 | 73 | 85 | 2.7 |
| 476455 | 5 | 19 | 34 | 56 | 80 | 4.6 |
| 476458 | 36 | 72 | 83 | 93 | 96 | 1.1 |
| 476459 | 23 | 59 | 75 | 85 | 91 | 1.5 |
| 476469 | 17 | 27 | 47 | 47 | 67 | 5.5 |
| 476473 | 0 | 6 | 32 | 50 | 68 | 6.2 |
| 476476 | 3 | 7 | 32 | 53 | 86 | 4.9 |

TABLE 50
Dose-dependent antisense inhibition
of human HTT in GM02171 cells

| ISIS | 750 | 1500 | 3000 | 6000 | 12000 | IC₅₀ |
|---|---|---|---|---|---|---|
| No. | nM | nM | nM | nM | nM | (μM) |
| 387916 | 59 | 79 | 93 | 98 | 98 | <0.8 |
| 463566 | 4 | 33 | 42 | 62 | 79 | 3.8 |
| 463567 | 38 | 41 | 69 | 85 | 94 | 1.5 |
| 463568 | 21 | 26 | 41 | 58 | 64 | 4.8 |
| 463571 | 8 | 23 | 56 | 63 | 75 | 3.7 |
| 476441 | 0 | 13 | 7 | 0 | 12 | >12.0 |
| 476444 | 11 | 0 | 0 | 67 | 59 | 8.8 |
| 476449 | 4 | 27 | 37 | 51 | 63 | 5.8 |
| 476453 | 6 | 40 | 40 | 51 | 73 | 4.9 |
| 476455 | 32 | 15 | 18 | 47 | 61 | 7.8 |
| 476458 | 42 | 54 | 71 | 86 | 84 | 1.2 |
| 476459 | 22 | 38 | 70 | 44 | 73 | 4.3 |
| 476469 | 7 | 24 | 30 | 56 | 58 | 7.8 |
| 476473 | 4 | 10 | 15 | 33 | 43 | >12.0 |
| 476476 | 5 | 16 | 18 | 23 | 41 | >12.0 |

TABLE 51
Dose-dependent antisense inhibition
of human HTT in GM02171 cells

| ISIS | 750 | 1500 | 3000 | 6000 | 12000 | IC₅₀ |
|---|---|---|---|---|---|---|
| No. | nM | nM | nM | nM | nM | (μM) |
| 387916 | 66 | 89 | 95 | 97 | 99 | <0.8 |
| 463566 | 32 | 55 | 76 | 77 | 93 | 1.3 |
| 463567 | 51 | 61 | 87 | 94 | 97 | 0.7 |
| 463568 | 26 | 23 | 72 | 87 | 94 | 1.6 |
| 463571 | 32 | 34 | 60 | 86 | 94 | 1.9 |
| 476441 | 18 | 18 | 27 | 47 | 44 | >12.0 |
| 476444 | 15 | 0 | 31 | 51 | 58 | 7.1 |
| 476449 | 27 | 33 | 56 | 80 | 81 | 2.6 |
| 476453 | 24 | 28 | 55 | 75 | 83 | 2.7 |
| 476455 | 24 | 26 | 52 | 55 | 73 | 3.7 |
| 476458 | 63 | 77 | 87 | 89 | 94 | 0.2 |
| 476459 | 37 | 55 | 56 | 62 | 86 | 1.5 |
| 476469 | 22 | 41 | 40 | 63 | 76 | 2.9 |
| 476473 | 7 | 28 | 33 | 51 | 73 | 5.0 |
| 476476 | 11 | 29 | 26 | 55 | 69 | 4.6 |

### Example 9: Antisense Inhibition of Human HTT in GM04281 Cells by Oligonucleotides Designed by Microwalk

Additional gapmers were designed based on the gapmers selected from studies described in Example 4. These gapmers were designed by creating gapmers shifted slightly upstream and downstream (i.e. “microwalk”) of the original gapmers from Tables 8, 9, and 10. Gapmers were also created with 3-9-3 or 5-9-5 motifs, and with constrained 6(S)—CH₃-bicyclic nucleic acid (BNA) molecules at various nucleoside positions.

These gapmers were tested in vitro. Cultured GM04281 cells at a density of 25,000 cells per well were transfected using electroporation with 5,000 nM antisense oligonucleotide. After a treatment period of approximately 24 hours, RNA was isolated from the cells and HTT mRNA levels were measured by quantitative real-time PCR. HTT mRNA levels were adjusted according to total RNA content, as measured by RIBOGREEN. Results are presented as percent inhibition of HTT mRNA, relative to untreated control cells.

The chimeric antisense oligonucleotides in Tables 52-56 were designed as 3-9-3 or 5-9-5 gapmers. The parent gapmers, ISIS 435869, ISIS 435870, ISIS 435874, ISIS 435879, and ISIS 435890, from which the newly designed gapmers were derived are marked with an asterisk (*) in the table. ISIS 387916 was included in the study as a benchmark oligonucleotide against which the potency of the antisense oligonucleotides targeting nucleotides overlapping each SNP position could be compared.

The 3-9-3 gapmers are 15 nucleotides in length, wherein the central gap segment is comprised of nine 2′-deoxynucleosides and is flanked on both 5′ and 3′ directions by wings comprising 3 sugar modified nucleosides each.

The 5-9-5 gapmers are 19 nucleotides in length, wherein the central gap segment is comprised of nine 2′-deoxynucleosides and is flanked on both 5′ and 3′ directions by wings comprising 5 sugar modified nucleosides each.

The internucleoside linkages throughout each gapmer are phosphorothioate (P═S) linkages. All cytosine nucleobases throughout each gapmer are 5-methylcytosines. Bolded and underlined nucleotides in Tables 52-56 indicate the positions of the 6(S)—CH₃-BNA molecules (e.g. cEt molecules) in each gapmer. Italicized nucleotides are MOE subunits.

“Start site” indicates the 5′-most nucleotide to which the gapmer is targeted. “Stop site” indicates the 3′-most nucleotide to which the gapmer is targeted. ‘Target allele’ indicates whether the gapmer is targeted to the major or the minor allele. The number in parentheses indicates the position on the oligonucleotide opposite to the SNP position.

TABLE 52
Comparison of inhibition of human HTT mRNA levels by
ISIS 387916 and chimeric antisense oligonucleotides targeted to
SNP rs4690072 (nucleobases 62147 to 62173 of SEQ ID NO: 1)

| | | | | | | | SEQ |
|---|---|---|---|---|---|---|---|
| Start | Stop | Target | ISIS | | | % | ID |
| Site | Site | allele | No. | Sequence | Motif | inhibition | NO |
| 145466 | 145485 | n/a | 387916 | TCTCTATTGC | 5-10-5 | 97 | 6 |
| | | | | ACATTCCAAG | | | |
| 62147 | 62165 | Major (6) | 460266 | GTGCTACCCA | 5-9-5 | 63 | 169 |
| | | | | ACCTTTCTG | | | |
| 62151 | 62169 | Major (10) | *435874 | CACAGTGCTA | 5-9-5 | 50 | 28 |
| | | | | CCCAACCTT | | | |
| 62151 | 62169 | Major (10) | 460213 | CACAGTGCTA | 5-9-5 | 22 | 28 |
| | | | | CCCAACCTT | | | |
| 62151 | 62169 | Major (10) | 460220 | CACAGTGCTA | 5-9-5 | 24 | 28 |
| | | | | CCCAACCTT | | | |
| 62151 | 62169 | Major (10) | 460221 | CACAGTGCTA | 5-9-5 | 28 | 28 |
| | | | | CCCAACCTT | | | |
| 62153 | 62167 | Major (8) | 460208 | CAGTGCTACC | 3-9-3 | 81 | 177 |
| | | | | CAACC | | | |
| 62155 | 62173 | Major (14) | 460267 | ATATCACAGT | 5-9-5 | 37 | 180 |
| | | | | GCTACCCAA | | | |

TABLE 53
Comparison of inhibition of human HTT mRNA levels by
ISIS 387916 and chimeric antisense oligonucleotides targeted to
SNP rs2298969 (nucleobases 125884 to 125910 of SEQ ID NO: 1)

| | | | | | | | SEQ |
|---|---|---|---|---|---|---|---|
| Start | Stop | Target | ISIS | | | % | ID |
| Site | Site | allele | No. | Sequence | Motif | inhibition | NO |
| 145466 | 145485 | n/a | 387916 | TCTCTATTGC | 5-10-5 | 97 | 6 |
| | | | | ACATTCCAAG | | | |
| 125884 | 125902 | Minor (6) | 460233 | GATGCTGACT | 5-9-5 | 76 | 182 |
| | | | | TGGGCCATT | | | |
| 125888 | 125906 | Minor (10) | *435890 | AAGGGATGCT | 5-9-5 | 75 | 94 |
| | | | | GACTTGGGC | | | |
| 125888 | 125906 | Minor (10) | 460215 | AAGGGATGCT | 5-9-5 | 26 | 94 |
| | | | | GACTTGGGC | | | |
| 125888 | 125906 | Minor (10) | 460224 | AAGGGATGCT | 5-9-5 | 38 | 94 |
| | | | | GACTTGGGC | | | |
| 125888 | 125906 | Minor (10) | 460225 | AAGGGATGCT | 5-9-5 | 49 | 94 |
| | | | | GACTTGGGC | | | |
| 125890 | 125904 | Minor (8) | 460210 | GGGATGCTGA | 3-9-3 | 97 | 189 |
| | | | | CTTGG | | | |
| 125892 | 125910 | Minor (14) | 460229 | TGCCAAGGGA | 5-9-5 | 60 | 192 |
| | | | | TGCTGACTT | | | |

TABLE 54
Comparison of inhibition of human HTT mRNA levels by
ISIS 387916 and chimeric antisense oligonucleotides targeted to
SNP rs7685686 (nucleobases 146782 to 146808 of SEQ ID NO: 1)

| | | | | | | | SEQ |
|---|---|---|---|---|---|---|---|
| Start | Stop | Target | ISIS | | | % | ID |
| Site | Site | allele | No. | Sequence | Motif | inhibition | NO |
| 145466 | 145485 | n/a | 387916 | TCTCTATTGC | 5-10-5 | 97 | 6 |
| | | | | ACATTCCAAG | | | |
| 146782 | 146800 | Major (6) | 460232 | AATTGTCATC | 5-9-5 | 82 | 195 |
| | | | | ACCAGAAAA | | | |
| 146786 | 146804 | Major (10) | *435879 | AATAAATTGT | 5-9-5 | 84 | 99 |
| | | | | CATCACCAG | | | |
| 146786 | 146804 | Major (10) | 460214 | AATAAATTGT | 5-9-5 | 33 | 99 |
| | | | | CATCACCAG | | | |
| 146786 | 146804 | Major (10) | 460222 | AATAAATTGT | 5-9-5 | 87 | 99 |
| | | | | CATCACCAG | | | |
| 146786 | 146804 | Major (10) | 460223 | AATAAATTGT | 5-9-5 | 75 | 99 |
| | | | | CATCACCAG | | | |
| 146788 | 146802 | Major (8) | 460209 | TAAATTGTCA | 3-9-3 | 96 | 203 |
| | | | | TCACC | | | |
| 146790 | 146808 | Major (14) | 460228 | TATTAATAAA | 5-9-5 | 0 | 206 |
| | | | | TTGTCATCA | | | |

TABLE 55
Comparison of inhibition of human HTT mRNA levels by
ISIS 387916 and chimeric antisense oligonucleotides targeted to
SNP rs362331 (nucleobases 155475 to 155501 of SEQ ID NO: 1)

| | | | | | | | SEQ |
|---|---|---|---|---|---|---|---|
| Start | Stop | Target | ISIS | | | % | ID |
| Site | Site | allele | No. | Sequence | Motif | inhibition | NO |
| 145466 | 145485 | n/a | 387916 | TCTCTATTGC | 5-10-5 | 97 | 6 |
| | | | | ACATTCCAAG | | | |
| 155475 | 155493 | Major (6) | 460231 | ACAGTAGATG | 5-9-5 | 88 | 209 |
| | | | | AGGGAGCAG | | | |
| 155479 | 155497 | Major (10) | *435870 | GCACACAGTA | 5-9-5 | 86 | 103 |
| | | | | GATGAGGGA | | | |
| 155479 | 155497 | Major (10) | 460212 | GCACACAGTA | 5-9-5 | 89 | 103 |
| | | | | GATGAGGGA | | | |
| 155479 | 155497 | Major (10) | 460218 | GCACACAGTA | 5-9-5 | 90 | 103 |
| | | | | GATGAGGGA | | | |
| 155479 | 155497 | Major (10) | 460219 | GCACACAGTA | 5-9-5 | 88 | 103 |
| | | | | GATGAGGGA | | | |
| 155481 | 155495 | Major (8) | 460207 | ACACAGTAGA | 3-9-3 | 89 | 217 |
| | | | | TGAGG | | | |
| 155483 | 155501 | Major (14) | 460227 | AAGTGCACAC | 5-9-5 | 45 | 220 |
| | | | | AGTAGATGA | | | |

TABLE 56
Comparison of inhibition of human HTT mRNA levels by
ISIS 387916 and chimeric antisense oligonucleotides targeted to
SNP rs362306 (nucleobases 181740 to 181766 of SEQ ID NO: 1)

| | | | | | | | SEQ |
|---|---|---|---|---|---|---|---|
| Start | Stop | Target | ISIS | | | % | ID |
| Site | Site | allele | No. | Sequence | Motif | inhibition | NO |
| 145466 | 145485 | n/a | 387916 | TCTCTATTGC | 5-10-5 | 97 | 6 |
| | | | | ACATTCCAAG | | | |
| 181740 | 181758 | Major (6) | 460230 | AGCTGCAACC | 5-9-5 | 66 | 223 |
| | | | | TGGCAACAA | | | |
| 181744 | 181762 | Major (10) | *435869 | GAGCAGCTGC | 5-9-5 | 69 | 149 |
| | | | | AACCTGGCA | | | |
| 181744 | 181762 | Major (10) | 460211 | GAGCAGCTGC | 5-9-5 | 22 | 149 |
| | | | | AACCTGGCA | | | |
| 181744 | 181762 | Major (10) | 460216 | GAGCAGCTGC | 5-9-5 | 18 | 149 |
| | | | | AACCTGGCA | | | |
| 181744 | 181762 | Major (10) | 460217 | GAGCAGCTGC | 5-9-5 | 56 | 149 |
| | | | | AACCTGGCA | | | |
| 181746 | 181760 | Major (8) | 460206 | GCAGCTGCAA | 3-9-3 | 83 | 231 |
| | | | | CCTGG | | | |
| 181748 | 181766 | Major (14) | 460226 | GCAAGAGCAG | 5-9-5 | 51 | 234 |
| | | | | CTGCAACCT | | | |

### Example 10: Dose-Dependent Antisense Inhibition of Human Huntingtin mRNA Levels in Coriell Fibroblast Cell Lines

Gapmers from studies described in Example 9 were selected and tested at various doses in GM04281, GM02171 and GM02173B cell lines. Each cell line was plated at a density of 25,000 cells per well and transfected using electroporation with 312.5 nM, 625 nM, 1,250 nM, 2,500 nM, 5,000 nM and 10,000 nM concentrations of antisense oligonucleotide, as specified in Tables 75, 58, and 59. After a treatment period of approximately 16 hours, RNA was isolated from the cells and HTT mRNA levels were measured by quantitative real-time PCR. Human HTT primer probe set RTS2617 was used to measure mRNA levels. HTT mRNA levels were adjusted according to total RNA content, as measured by RIBOGREEN. Results are presented as percent inhibition of HTT mRNA, relative to untreated control cells. IC₅₀ values are also provided in Tables 57, 58, and 59.

TABLE 57
Dose-dependent antisense inhibition
of human HTTin GM04281 cells

| ISIS | 312.5 | 625 | 1,250 | 2,500 | 5,000 | 10,000 | IC₅₀ |
|---|---|---|---|---|---|---|---|
| No. | nM | nM | nM | nM | nM | nM | (μM) |
| 387916 | 26 | 49 | 68 | 86 | 94 | 97 | 0.7 |
| 435869 | 0 | 0 | 23 | 48 | 62 | 82 | 3.2 |
| 435870 | 15 | 38 | 50 | 65 | 85 | 88 | 1.3 |
| 435874 | 14 | 22 | 32 | 49 | 65 | 73 | 2.7 |
| 435879 | 0 | 17 | 40 | 61 | 83 | 94 | 1.8 |
| 435890 | 5 | 13 | 37 | 56 | 70 | 82 | 2.3 |
| 460206 | 10 | 18 | 37 | 52 | 66 | 85 | 2.3 |
| 460207 | 20 | 27 | 50 | 65 | 80 | 91 | 1.4 |
| 460208 | 21 | 34 | 51 | 63 | 70 | 79 | 1.5 |
| 460209 | 52 | 74 | 89 | 94 | 94 | 95 | 0.2 |
| 460210 | 34 | 61 | 84 | 91 | 97 | 98 | 0.5 |
| 460212 | 13 | 31 | 50 | 62 | 75 | 82 | 1.6 |
| 460218 | 14 | 27 | 50 | 63 | 78 | 86 | 1.8 |
| 460219 | 9 | 32 | 42 | 64 | 77 | 87 | 1.6 |
| 460222 | 19 | 21 | 42 | 57 | 73 | 78 | 1.7 |
| 460231 | 12 | 24 | 41 | 57 | 71 | 84 | 1.9 |
| 460233 | 16 | 28 | 59 | 66 | 72 | 74 | 1.8 |
| 460266 | 4 | 17 | 32 | 48 | 60 | 75 | 2.9 |

TABLE 58
Dose-dependent antisense inhibition
of human HTT in GM02171 cells

| ISIS | 312.5 | 625 | 1,250 | 2,500 | 5,000 | 10,000 | IC₅₀ |
|---|---|---|---|---|---|---|---|
| No. | nM | nM | nM | nM | nM | nM | (μM) |
| 387916 | 32 | 56 | 77 | 89 | 95 | 97 | 0.7 |
| 435869 | 0 | 6 | 22 | 40 | 69 | 84 | 2.9 |
| 435870 | 15 | 19 | 32 | 51 | 68 | 77 | 2.4 |
| 435874 | 0 | 5 | 1 | 17 | 17 | 30 | >10.0 |
| 435879 | 0 | 8 | 0 | 16 | 36 | 47 | 15.3 |
| 435890 | 14 | 16 | 19 | 19 | 39 | 57 | 9.3 |
| 460206 | 5 | 13 | 33 | 41 | 68 | 80 | 2.7 |
| 460207 | 13 | 10 | 22 | 22 | 33 | 39 | 45.6 |
| 460208 | 13 | 15 | 11 | 11 | 15 | 53 | 10.8 |
| 460209 | 8 | 27 | 46 | 70 | 80 | 86 | 1.6 |
| 460210 | 19 | 37 | 55 | 75 | 88 | 96 | 1.1 |
| 460212 | 8 | 23 | 30 | 43 | 57 | 74 | 2.2 |
| 460218 | 15 | 26 | 27 | 36 | 52 | 78 | 3.2 |
| 460219 | 16 | 17 | 32 | 44 | 69 | 76 | 2.5 |
| 460222 | 14 | 3 | 0 | 0 | 13 | 0 | >10.0 |
| 460231 | 6 | 8 | 13 | 16 | 33 | 56 | 10.4 |
| 460233 | 27 | 30 | 39 | 46 | 61 | 73 | 2.4 |
| 460266 | 0 | 15 | 20 | 15 | 18 | 34 | >10.0 |

TABLE 59
Dose-dependent antisense inhibition
of human HTT in GM02173B cells

| ISIS | 312.5 | 625 | 1,250 | 2,500 | 5,000 | 10,000 | IC₅₀ |
|---|---|---|---|---|---|---|---|
| No. | nM | nM | nM | nM | nM | nM | (μM) |
| 387916 | 22 | 47 | 76 | 88 | 96 | 98 | 0.7 |
| 435869 | 10 | 0 | 16 | 38 | 59 | 76 | 3.9 |
| 435870 | 22 | 36 | 44 | 58 | 69 | 81 | 2.0 |
| 435874 | 11 | 6 | 25 | 23 | 32 | 42 | >10.0 |
| 435879 | 0 | 9 | 21 | 30 | 52 | 68 | 4.8 |
| 435890 | 12 | 16 | 30 | 31 | 48 | 66 | 4.5 |
| 460206 | 11 | 13 | 18 | 35 | 59 | 74 | 3.5 |
| 460207 | 15 | 25 | 30 | 37 | 42 | 66 | 4.3 |
| 460208 | 5 | 14 | 27 | 32 | 52 | 51 | 9.0 |
| 460209 | 27 | 49 | 61 | 79 | 81 | 74 | 0.8 |
| 460210 | 19 | 40 | 61 | 77 | 89 | 95 | 1.0 |
| 460212 | 0 | 19 | 32 | 32 | 61 | 78 | 2.9 |
| 460218 | 4 | 17 | 26 | 38 | 64 | 82 | 3.0 |
| 460219 | 5 | 6 | 26 | 47 | 68 | 84 | 2.9 |
| 460222 | 13 | 19 | 23 | 30 | 35 | 50 | 16.1 |
| 460231 | 7 | 33 | 25 | 35 | 54 | 77 | 3.7 |
| 460233 | 11 | 20 | 37 | 52 | 68 | 69 | 2.3 |
| 460266 | 12 | 6 | 10 | 21 | 25 | 47 | >10.0 |

### Example 11: Dose-Dependent Antisense Inhibition of Human HTT in GM04281 and GM02171 Cells by Oligonucleotides Designed by Microwalk

Additional gapmers were designed based on the gapmers selected from studies described in Example 10. These gapmers were designed by creating gapmers shifted slightly upstream and downstream (i.e. “microwalk”) of the original gapmers from Tables 57, 58, and 59. Gapmers were also created with 4-9-4 MOE or 5-9-5 MOE motifs, and with constrained 6(S)—CH₃-bicyclic nucleic acid (BNA) molecules at various nucleotide positions.

These gapmers were tested in the GM04281 and GM02171 cell lines. Cultured GM04281 or GM02171 cells at a density of 25,000 cells per well were transfected using electroporation with 2,500 nM or 5,000 nM antisense oligonucleotide. After a treatment period of approximately 24 hours, RNA was isolated from the cells and HTT mRNA levels were measured by quantitative real-time PCR. HTT mRNA levels were adjusted according to total RNA content, as measured by RIBOGREEN. Results are presented as percent inhibition of HTT mRNA, relative to untreated control cells.

The chimeric antisense oligonucleotides in Tables 60, 61, and 62 were designed as 3-9-3, 4-9-4, or 5-9-5 MOE gapmers. The parent gapmers, ISIS 435890, ISIS 460210, ISIS 435879, ISIS 460209, ISIS 435870, and ISIS 460207, from which the newly designed gapmers were derived are marked with an asterisk (*) in the table. ISIS 387916 was included in the study as a benchmark oligonucleotide against which the potency of the antisense oligonucleotides targeting nucleotides overlapping each SNP position could be compared.

The 3-9-3 gapmers are 15 nucleotides in length, wherein the central gap segment is comprised of nine 2′-deoxynucleotides and is flanked on both 5′ and 3′ directions by wings comprising 3 nucleotides each.

The 4-9-4 gapmers are 17 nucleotides in length, wherein the central gap segment is comprised of nine 2′-deoxynucleotides and is flanked on both 5′ and 3′ directions by wings comprising 4 nucleotides each.

The 5-9-5 gapmers are 19 nucleotides in length, wherein the central gap segment is comprised of nine 2′-deoxynucleotides and is flanked on both 5′ and 3′ directions by wings comprising 5 nucleotides each.

The internucleoside linkages throughout each gapmer are phosphorothioate (P═S) linkages. All cytosine nucleobases throughout each gapmer are 5-methylcytosines. Bolded and underlined nucleotides in Tables 60, 61, and 62 indicate the positions of the 6 (S)—CH₃-BNA (e.g. cEt molecules) molecules in each gapmer. Italicized nucleotides are MOE subunits.

The gapmers are organized in Tables 60, 61, and 62, according to the SNP site they target. “Start site” indicates the 5′-most nucleotide to which the gapmer is targeted. “Stop site” indicates the 3′-most nucleotide to which the gapmer is targeted. ‘Target allele’ indicates whether the gapmer is targeted to the major or the minor allele. The number in parentheses indicates the position on the oligonucleotide opposite to the SNP position.

TABLE 60
Comparison of inhibition of human HTT mRNA levels by
ISIS 387916 and chimeric antisense oligonucleotides targeted to
SNP rs2298969 (nucleobases 125888 to 125907 of SEQ ID NO: 1)

| | | | | | Concen- | % in- | % in- | SEQ |
|---|---|---|---|---|---|---|---|---|
| Start | Stop | ISIS | | | tration | hibition | hibition | ID |
| position | position | No. | Sequence | Motif | (nM) | in GM04281 | in GM02171 | NO |
| 145466 | 145485 | 387916 | TCTCTATTGCA | 5-10-5 | 5000 | 57 | 24 | 6 |
| | | | CATTCCAAG | | | | | |
| 125888 | 125907 | *435890 | AAGGGATGCTG | 5-9-5 | 2500 | 22 | 0 | 94 |
| | | | ACTTGGGC | | 5000 | 41 | 23 | |
| 125890 | 125904 | *460210 | GGGATGCTGAC | 3-9-3 | 2500 | 59 | 24 | 189 |
| | | | TTGG | | 5000 | 81 | 33 | |
| 125889 | 125905 | 474870 | AGGGATGCTG | 4-9-4 | 2500 | 23 | 3 | 187 |
| | | | ACTTGGG | | 5000 | 44 | 34 | |
| 125889 | 125905 | 474890 | AGGGATGCTG | 4-9-4 | 2500 | 38 | 6 | 187 |
| | | | ACTTGGG | | 5000 | 49 | 25 | |
| 125889 | 125905 | 474910 | AGGGATGCTGA | 4-9-4 | 2500 | 34 | 8 | 187 |
| | | | CTTGGG | | 5000 | 49 | 41 | |
| 125889 | 125905 | 474914 | AGGGATGCTGA | 4-9-4 | 2500 | 44 | 14 | 187 |
| | | | CTTGGG | | 5000 | 44 | 21 | |
| 125888 | 125907 | 474918 | AAGGGATGCT | 5-9-5 | 2500 | 31 | 0 | 94 |
| | | | GACTTGGGC | | 5000 | 26 | 25 | |
| 125888 | 125907 | 474922 | AAGGGATGCT | 5-9-5 | 2500 | 33 | 14 | 94 |
| | | | GACTTGGGC | | 5000 | 65 | 24 | |
| 125889 | 125905 | 476332 | AGGGATGCTG | 4-9-4 | 2500 | 23 | 13 | 187 |
| | | | ACTTGGG | | 5000 | 51 | 42 | |
| 125888 | 125907 | 476336 | AAGGGATGCTG | 5-9-5 | 2500 | 5 | 0 | 94 |
| | | | ACTTGGGC | | 5000 | 43 | 9 | |

TABLE 61
Comparison of inhibition of human HTT mRNA levels by
ISIS 387916 and chimeric antisense oligonucleotides targeted to
SNP rs7685686 (nucleobases 146786 to 146805 of SEQ ID NO: 1)

| | | | | | Concen- | % | % | SEQ |
|---|---|---|---|---|---|---|---|---|
| Start | Stop | ISIS | | | tration | inhibition | inhibition | ID |
| position | position | No. | Sequence | Motif | (nM) | in GM04281 | in GM02171 | NO |
| 145466 | 145485 | 387916 | TCTCTATTGCA | 5-10-5 | 5000 | 57 | 24 | 6 |
| | | | CATTCCAAG | | | | | |
| 146786 | 146805 | *435879 | AATAAATTGTC | 5-9-5 | 2500 | 39 | 0 | 99 |
| | | | ATCACCAG | | 5000 | 59 | 19 | |
| 146788 | 146802 | *460209 | TAAATTGTCAT | 3-9-3 | 2500 | 3 | 0 | 203 |
| | | | CACC | | 5000 | 13 | 5 | |
| 146787 | 146803 | 474871 | ATAAATTGTCA | 4-9-4 | 2500 | 82 | 32 | 200 |
| | | | TCACCA | | 5000 | 83 | 58 | |
| 146787 | 146803 | 474891 | ATAAATTGTCA | 4-9-4 | 2500 | 84 | 29 | 200 |
| | | | TCACCA | | 5000 | 89 | 56 | |
| 146787 | 146803 | 474911 | ATAAATTGTCA | 4-9-4 | 2500 | 70 | 18 | 200 |
| | | | TCACCA | | 5000 | 83 | 40 | |
| 146787 | 146803 | 474915 | ATAAATTGTCA | 4-9-4 | 2500 | 38 | 9 | 200 |
| | | | TCACCA | | 5000 | 74 | 14 | |
| 146786 | 146805 | 474919 | AATAAATTGTC | 5-9-5 | 2500 | 80 | 7 | 99 |
| | | | ATCACCAG | | 5000 | 84 | 37 | |
| 146786 | 146805 | 474923 | AATAAATTGTC | 5-9-5 | 2500 | 74 | 32 | 99 |
| | | | ATCACCAG | | 5000 | 83 | 51 | |
| 146787 | 146803 | 476333 | ATAAATTGTCA | 4-9-4 | 2500 | 75 | 28 | 200 |
| | | | TCACCA | | 5000 | 86 | 21 | |
| 146786 | 146805 | 476337 | AATAAATTGTC | 5-9-5 | 2500 | 71 | 6 | 99 |
| | | | ATCACCAG | | 5000 | 83 | 31 | |

TABLE 62
Comparison of inhibition of human HTT mRNA levels by
ISIS 387916 and chimeric antisense oligonucleotides targeted to
SNP rs362331 (nucleobases 155478 to 155498 of SEQ ID NO: 1)

| | | | | | Concen- | % | % | SEQ |
|---|---|---|---|---|---|---|---|---|
| Start | Stop | ISIS | | | tration | inhibition | inhibition | ID |
| position | position | No. | Sequence | Motif | (nM) | in GM04281 | in GM02171 | NO |
| 145466 | 145485 | 387916 | TCTCTATTGCAC | 5-10-5 | 5000 | 57 | 24 | 6 |
| | | | ATTCCAAG | | | | | |
| 155479 | 155498 | *435870 | GCACACAGTAG | 5-9-5 | 2500 | 19 | 1 | 103 |
| | | | ATGAGGGA | | 5000 | 49 | 34 | |
| 155481 | 155495 | *460207 | ACACAGTAGAT | 3-9-3 | 2500 | 0 | 0 | 217 |
| | | | GAGG | | 5000 | 7 | 8 | |
| 155480 | 155496 | 474872 | CACACAGTAGA | 4-9-4 | 2500 | 35 | 9 | 214 |
| | | | TGAGGG | | 5000 | 63 | 37 | |
| 155480 | 155496 | 474892 | CACACAGTAGA | 4-9-4 | 2500 | 43 | 16 | 214 |
| | | | TGAGGG | | 5000 | 69 | 31 | |
| 155480 | 155496 | 474912 | CACACAGTAGA | 4-9-4 | 2500 | 16 | 9 | 214 |
| | | | TGAGGG | | 5000 | 36 | 6 | |
| 155480 | 155496 | 474916 | CACACAGTAGA | 4-9-4 | 2500 | 22 | 5 | 214 |
| | | | TGAGGG | | 5000 | 47 | 7 | |
| 155479 | 155498 | 474920 | GCACACAGTAG | 5-9-5 | 2500 | 19 | 0 | 103 |
| | | | ATGAGGGA | | 5000 | 43 | 23 | |
| 155479 | 155498 | 474924 | GCACACAGTAG | 5-9-5 | 2500 | 29 | 8 | 103 |
| | | | ATGAGGGA | | 5000 | 48 | 22 | |
| 155480 | 155496 | 476334 | CACACAGTAGA | 4-9-4 | 2500 | 35 | 7 | 214 |
| | | | TGAGGG | | 5000 | 62 | 32 | |
| 155479 | 155498 | 476338 | GCACACAGTAG | 5-9-5 | 2500 | 26 | 9 | 103 |
| | | | ATGAGGGA | | 5000 | 40 | 4 | |
| 155479 | 155495 | 474873 | ACACAGTAGAT | 4-9-4 | 2500 | 53 | 9 | 285 |
| | | | GAGGGA | | 5000 | 61 | 29 | |
| 155479 | 155495 | 474893 | ACACAGTAGAT | 4-9-4 | 2500 | 47 | 5 | 285 |
| | | | GAGGGA | | 5000 | 59 | 30 | |
| 155479 | 155495 | 474913 | ACACAGTAGAT | 4-9-4 | 2500 | 30 | 16 | 285 |
| | | | GAGGGA | | 5000 | 29 | 17 | |
| 155479 | 155495 | 474917 | ACACAGTAGAT | 4-9-4 | 2500 | 23 | 12 | 285 |
| | | | GAGGGA | | 5000 | 40 | 5 | |
| 155478 | 155497 | 474921 | CACACAGTAGA | 5-9-5 | 2500 | 28 | 0 | 212 |
| | | | TGAGGGAG | | 5000 | 43 | 23 | |
| 155478 | 155497 | 474925 | CACACAGTAGA | 5-9-5 | 2500 | 30 | 9 | 212 |
| | | | TGAGGGAG | | 5000 | 61 | 34 | |
| 155479 | 155495 | 476335 | ACACAGTAGAT | 4-9-4 | 2500 | 35 | 2 | 285 |
| | | | GAGGGA | | 5000 | 53 | 31 | |
| 155478 | 155497 | 476339 | CACACAGTAGA | 5-9-5 | 2500 | 15 | 0 | 212 |
| | | | TGAGGGAG | | 5000 | 34 | 13 | |

### Example 12: Dose-Dependent Antisense Inhibition of Human Huntingtin mRNA Levels in Coriell Fibroblast Cell Lines

Gapmers from the studies described in Example 11 were selected and tested at various doses in GM04281, GM02171 and GM02173B cell lines. Each cell line was plated at a density of 25,000 cells per well and transfected using electroporation with 625 nM, 1,250 nM, 2,500 nM, 5,000 nM and 10,000 nM concentrations of antisense oligonucleotide, as specified in Tables 63, 64, and 65. After a treatment period of approximately 16 hours, RNA was isolated from the cells and HTT mRNA levels were measured by quantitative real-time PCR. Human HTT primer probe set RTS2617 was used to measure mRNA levels. HTT mRNA levels were adjusted according to total RNA content, as measured by RIBOGREEN. Results are presented as percent inhibition of HTT mRNA, relative to untreated control cells. IC₅₀ values are also provided in Tables 63, 64, and 65.

TABLE 63
Dose-dependent antisense inhibition
of human HTT in GM04281 cells

| ISIS | 625 | 1250 | 2500 | 5000 | 10000 | IC₅₀ |
|---|---|---|---|---|---|---|
| No | nM | nM | nM | nM | nM | (μM) |
| 387916 | 70 | 83 | 94 | 96 | 98 | <0.6 |
| 460207 | 51 | 63 | 83 | 91 | 93 | 0.5 |
| 460209 | 83 | 93 | 96 | 97 | 97 | <0.6 |
| 460210 | 70 | 89 | 94 | 97 | 98 | 0.6 |
| 474871 | 94 | 97 | 96 | 96 | 95 | <0.6 |
| 474873 | 51 | 73 | 89 | 94 | 95 | 0.5 |
| 474891 | 93 | 95 | 97 | 96 | 95 | <0.6 |
| 474892 | 48 | 72 | 89 | 93 | 95 | 0.6 |
| 474911 | 85 | 92 | 96 | 95 | 94 | <0.6 |
| 474919 | 89 | 94 | 95 | 94 | 96 | <0.6 |
| 474922 | 21 | 47 | 73 | 86 | 96 | 1.5 |
| 474923 | 86 | 94 | 96 | 95 | 94 | <0.6 |
| 476333 | 92 | 94 | 95 | 95 | 96 | <0.6 |
| 476334 | 45 | 70 | 87 | 92 | 95 | 0.6 |
| 476337 | 83 | 92 | 95 | 96 | 96 | <0.6 |

TABLE 64
Dose-dependent antisense inhibition
of human HTT in GM02171 cells

| ISIS | 625 | 1250 | 2500 | 5000 | 10000 | IC₅₀ |
|---|---|---|---|---|---|---|
| No | nM | nM | nM | nM | nM | (μM) |
| 387916 | 28 | 38 | 63 | 82 | 99 | 1.6 |
| 460207 | 16 | 0 | 20 | 22 | 55 | 10.0 |
| 460209 | 27 | 50 | 61 | 87 | 94 | 9.9 |
| 460210 | 34 | 60 | 80 | 86 | 97 | 0.9 |
| 474871 | 62 | 74 | 84 | 87 | 90 | 0.1 |
| 474873 | 13 | 29 | 61 | 77 | 89 | 2.2 |
| 474891 | 57 | 72 | 80 | 83 | 88 | 0.2 |
| 474892 | 23 | 26 | 51 | 68 | 81 | 2.5 |
| 474911 | 47 | 58 | 68 | 72 | 82 | 0.7 |
| 474919 | 44 | 48 | 65 | 71 | 83 | 1.1 |
| 474922 | 15 | 27 | 49 | 74 | 79 | 2.6 |
| 474923 | 27 | 53 | 74 | 79 | 84 | 1.5 |
| 476333 | 42 | 53 | 75 | 76 | 84 | 1.0 |
| 476334 | 20 | 23 | 58 | 71 | 87 | 2.3 |
| 476337 | 23 | 34 | 60 | 62 | 75 | 2.7 |

TABLE 65
Dose-dependent antisense inhibition
of human HTT in GM02173B cells

| ISIS | 625 | 1250 | 2500 | 5000 | 10000 | IC₅₀ |
|---|---|---|---|---|---|---|
| No | nM | nM | nM | nM | nM | (μM) |
| 387916 | 38 | 75 | 89 | 95 | 99 | 0.9 |
| 460207 | 13 | 27 | 52 | 46 | 63 | 6.5 |
| 460209 | 79 | 68 | 84 | 90 | 92 | <0.6 |
| 460210 | 37 | 62 | 79 | 92 | 97 | 0.9 |
| 474871 | 74 | 83 | 87 | 92 | 89 | <0.6 |
| 474873 | 22 | 32 | 67 | 72 | 92 | 1.9 |
| 474891 | 69 | 78 | 84 | 89 | 89 | <0.6 |
| 474892 | 26 | 50 | 75 | 83 | 91 | 1.3 |
| 474911 | 50 | 66 | 76 | 86 | 86 | 0.6 |
| 474919 | 57 | 67 | 74 | 87 | 82 | <0.6 |
| 474922 | 15 | 32 | 61 | 71 | 90 | 2.2 |
| 474923 | 49 | 67 | 78 | 83 | 85 | 0.5 |
| 476333 | 58 | 71 | 78 | 87 | 89 | <0.6 |
| 476334 | 20 | 42 | 63 | 76 | 91 | 1.8 |
| 476337 | 48 | 63 | 71 | 79 | 80 | 0.6 |

### Example 13: Strategy for Selection of Antisense Oligonucleotides Based on Potency and Selectivity

Gapmers from each of the studies described above were selected for further analysis based on potency and selectivity.

Potency was based on the percent inhibition of HTT mRNA achieved by the antisense oligonucleotides targeting a SNP compared to the percent inhibition of HTT mRNA achieved by the benchmark oligonucleotide, ISIS 387916.

Selectivity was based on the ability of the antisense oligonucleotides targeting a SNP to inhibit expression of the major allele and not of the minor allele. The usage of the three cell lines with different genotypes at each SNP position facilitated this process.

ISIS 460065 (5′-ATAAATTGTCATCACCAG-3′ (SEQ ID NO: 199)) is a 4-9-5 MOE gapmer targeted to SNP rs7685686 (major allele A, minor allele G) at position 9 of the oligonucleotide. The GM04281 cell line is homozygous AA at SNP position rs7685686. The GM02173B cell line is heterozygous AG at SNP position rs7685686. The GM02171 cell line is homozygous GG at SNP position rs7685686. Therefore, selectivity is shown if ISIS 460065 causes potent inhibition of HTT mRNA in GM04281, less potent inhibition of HTT mRNA in GM02173, and little to no significant inhibition of HTT mRNA in GM02171. IC50 values taken from Table 20, 21, and 22, and presented below in Table 66, confirm varying degrees of inhibition in the three cell lines, wherein expression was most reduced in the homozygous AA cell line, moderately reduced in the heterozygous AG cell line, and less reduced in the homozygous GG cell line. IC50 is the concentration of antisense oligonucleotide required for 50 percent inhibition HTT mRNA. IC50 values are in μM.

TABLE 66
Genotype of the Coriell cell lines for SNP rs7685686 and comparison of
inhibition of HTT mRNA by ISIS 460065 in each cell line

| | GM04281 | GM02173B | GM02171 |
|---|---|---|---|
| Genotype | AA | AG | GG |
| IC₅₀ with ISIS | 1.1 | 3.6 | 10.3 |
| 460065 | | | |

ISIS 459978 (5′-ACAGTGCTACCCAACCT-3′ (SEQ ID NO: 174)) is a 2-9-6 MOE gapmer targeted to SNP rs4690072 (major allele T, minor allele G) at position 9 of the oligonucleotide. The GM04281 cell line is homozygous TT at SNP position rs4690072. The GM02173B cell line is heterozygous TG at SNP position rs4690072. The GM02171 cell line is homozygous GG at SNP position rs4690072. Therefore, selectivity is shown if ISIS 459978 causes potent inhibition of HTT mRNA in GM04281, less potent inhibition of HTT mRNA in GM02173, and little to no significant inhibition of HTT mRNA in GM02171. IC50 values taken from Table 20, 21, and 22, and presented below in Table 67, confirm varying degrees of inhibition in the three cell lines, wherein expression was most reduced in the homozygous TT cell line, moderately reduced in the heterozygous TG cell line, and less reduced in the homozygous GG cell line. IC50 is the concentration of antisense oligonucleotide required for 50 percent inhibition HTT mRNA. IC50 values are in μM.

TABLE 67
Genotype of the Coriell cell lines for SNP rs4690072 and comparison of
inhibition of HTT mRNA by ISIS 459978 in each cell line

| | GM04281 | GM02173B | GM02171 |
|---|---|---|---|
| Genotype | TT | TG | GG |
| IC₅₀ with ISIS | 2.5 | 8.4 | 12.7 |
| 459978 | | | |

ISIS 460028 (5′-GAGCAGCTGCAACCTGGCA-3′ (SEQ ID NO: 149)) is a 4-11-4 MOE gapmer targeted to SNP rs362306 (major allele G, minor allele A) at position 10 of the oligonucleotide. The GM04281 cell line is homozygous GG at SNP position rs362306. The GM02173B and GM02171 cell lines are heterozygous GA at SNP position rs362306. Therefore, selectivity is shown if ISIS 460028 causes potent inhibition of HTT mRNA in GM04281 and less potent inhibition of HTT mRNA in GM02173 and GM02171. IC50 values taken from Table 20, 21, and 22, and presented below in Table 68, confirm varying degrees of inhibition between the GM04281 cell line and the GM02173B and GM02171 cell lines, wherein expression was most reduced in the homozygous GG cell line and less reduced in the heterozygous AG cell line. IC50 is the concentration of antisense oligonucleotide required for 50 percent inhibition HTT mRNA. IC50 values are in μM.

TABLE 68
Genotype of the Coriell cell lines for SNP rs362306 and comparison of
inhibition of HTT mRNA by ISIS 460028 in each cell line

| | GM04281 | GM02173B | GM02171 |
|---|---|---|---|
| Genotype | GG | AG | AG |
| IC₅₀ with ISIS | 1.4 | 5.2 | 5.3 |
| 460028 | | | |

### Example 14: Strategy for Selection of Antisense Oligonucleotides with cEt Motifs Based on Potency and Selectivity

Gapmers from each of the studies described above were selected for further analysis based on potency and selectivity.

Potency was based on the percent inhibition of HTT mRNA achieved by the antisense oligonucleotides targeting a SNP compared to the percent inhibition of HTT mRNA achieved by the benchmark oligonucleotide, ISIS 387916.

Selectivity was based on the ability of the antisense oligonucleotides targeting a SNP to inhibit expression of the major allele and not of the minor allele. The usage of the three cell lines with different genotypes at each SNP position facilitated this process.

ISIS 460209 (5′-TAAATTGTCATCACC-3′ (SEQ ID NO: 203)) is a 3-9-3 gapmer with cEt subunits at positions 2, 3, 13, and 14, targeted to SNP rs7685686 (major allele A, minor allele G) at position 8 of the oligonucleotide. The GM04281 cell line is homozygous AA at SNP position rs7685686. The GM02173B cell line is heterozygous AG at SNP position rs7685686. The GM02171 cell line is homozygous GG at SNP position rs7685686. Therefore, selectivity is shown if ISIS 460209 causes potent inhibition of HTT mRNA in GM04281, less potent inhibition of HTT mRNA in GM02173, and little to no significant inhibition of HTT mRNA in GM02171. IC₅₀ values taken from Table 57, 58, and 59, and presented below in Table 69, confirm varying degrees of inhibition in the three cell lines, wherein expression was most reduced in the homozygous AA cell line, moderately reduced in the heterozygous AG cell line, and less reduced in the homozygous GG cell line. IC₅₀ is the concentration of antisense oligonucleotide required for 50 percent inhibition HTT mRNA. IC₅₀ values are in μM.

TABLE 69
Genotype of the Coriell cell lines for SNP rs7685686 and comparison of
inhibition of HTT mRNA by ISIS 460209 in each cell line

| | GM04281 | GM02173B | GM02171 |
|---|---|---|---|
| Genotype | AA | AG | GG |
| IC₅₀ with ISIS | 0.2 | 0.8 | 1.6 |
| 460209 | | | |

ISIS 460208 (5′-CAGTGCTACCCAACC-3′ (SEQ ID NO: 177)) is a 3-9-3 gapmer with cEt subunits at positions 2, 3, 13, and 14, targeted to SNP rs4690072 (major allele T, minor allele G) at position 8 of the oligonucleotide. The GM04281 cell line is homozygous TT at SNP position rs4690072. The GM02173B cell line is heterozygous TG at SNP position rs4690072. The GM02171 cell line is homozygous GG at SNP position rs4690072. Therefore, selectivity is shown if ISIS 460208 causes potent inhibition of HTT mRNA in GM04281, less potent inhibition of HTT mRNA in GM02173, and little to no significant inhibition of HTT mRNA in GM02171. IC₅₀ values taken from Table 57, 58, and 59, and presented below in Table 70, confirm varying degrees of inhibition in the three cell lines, wherein expression was most reduced in the homozygous TT cell line, moderately reduced in the heterozygous TG cell line, and less reduced in the homozygous GG cell line. IC₅₀ is the concentration of antisense oligonucleotide required for 50 percent inhibition HTT mRNA. IC₅₀ values are in μM.

TABLE 70
Genotype of the Coriell cell lines for SNP rs4690072 and comparison
of inhibition of HTT mRNA by ISIS 460208 in each cell line

| | GM04281 | GM02173B | GM02171 |
|---|---|---|---|
| Genotype | TT | TG | GG |
| IC₅₀ with ISIS | 1.5 | 9.0 | 10.8 |
| 460208 | | | |

ISIS 460206 (5′-GCAGCTGCAACCTGG-3′ (SEQ ID NO: 231)) is a 3-9-3 gapmer with cEt subunits at positions 2, 3, 13, and 14, targeted to SNP rs362306 (major allele G, minor allele A) at position 8 of the oligonucleotide. The GM04281 cell line is homozygous GG at SNP position rs362306. The GM02173B and GM02171 cell lines are heterozygous GA at SNP position rs362306. Therefore, selectivity is shown if ISIS 460206 causes potent inhibition of HTT mRNA in GM04281 and less potent inhibition of HTT mRNA in GM02173 and GM02171. IC₅₀ values taken from Table 57, 58, and 59, and presented below in Table 71, confirm varying degrees of inhibition between the GM04281 cell line and the GM02173B and GM02171 cell lines, wherein expression was most reduced in the homozygous GG cell line and less reduced in the heterozygous AG cell line. IC₅₀ is the concentration of antisense oligonucleotide required for 50 percent inhibition HTT mRNA. IC₅₀ values are in μM.

TABLE 71
Genotype of the Coriell cell lines for SNP rs362306 and comparison
of inhibition of HTT mRNA by ISIS 460206 in each cell line

| | GM04281 | GM02173B | GM02171 |
|---|---|---|---|
| Genotype | GG | AG | AG |
| IC₅₀ with ISIS | 2.3 | 2.7 | 2.7 |
| 460206 | | | |

### Example 15: Comparison of SNPs in Various Cell Lines and Mouse Models Associated with Huntington's Disease

The genotype at various SNP positions associated with Huntington's disease was compared amongst the three Corriell cell lines, used in the above Examples, as well as with the GM04022 fibroblast, the BACHD mouse model and the YAC18 mouse model.

The donor patient of the GM04022 fibroblast cell line was heterozygous at SNP position rs363125 (NCBI Entrez SNP database), harboring an A allele (adenine) and a C allele (cytosine) at nucleotide 5310 of SEQ ID NO: 2 (van Bilsen, P. H. J. et al., Human Gene Therapy. 19:710-718, 2008). YAC18 mice were developed with a YAC transgene containing human huntingtin gene (Hodgson, et al. Hum. Mol. Genet. 5:1875-85, 1996). BACHD mice were developed expressing a full-length mutant huntingtin gene with 97 glutamine repeats under the control of a bacterial artificial chromosome (Gray, M. et al., J. Neurosc. 28:6182-95, 2008). The comparative genotype at the indicated SNP positions in all four cell lines and mouse models is presented in Table 72.

TABLE 72
Genotypes of the Coriell cell lines and Huntington mouse models

| SNP | GM02171 | GM02173 | GM04281 | GM04022 | BACHD | YAC18 |
|---|---|---|---|---|---|---|
| rs3856973 | AA | AG | GG | AG | GG | AA |
| rs2285086 | GG | AG | AA | AG | AA | GG |
| rs7659144 | CG | CG | CC | CG | CC | GG |
| rs16843804 | TC | TC | CC | CC | CC | TT |
| rs2024115 | GG | AG | AA | AG | AA | GG |
| rs3733217 | CC | CC | CC | CC | CC | CC |
| rs10015979 | AA | AG | GG | AA | AA | AA |
| rs7691627 | AA | AG | GG | AG | GG | AA |
| rs2798235 | GG | GG | GG | AG | GG | GG |
| rs4690072 | GG | TG | TT | TG | TT | GG |
| rs6446723 | CC | TC | TT | TC | TT | CC |
| rs363081 | GG | GG | GG | GG | GG | GG |
| rs363080 | CC | CC | CC | TC | CC | CC |
| rs363075 | GG | GG | GG | GG | GG | GG |
| rs363064 | TC | TC | CC | CC | CC | TT |
| rs3025849 | AA | AA | AA | AA | AA | AA |
| rs363102 | AA | AA | AA | AG | AA | AA |
| rs11731237 | CC | TC | TT | CC | CC | CC |
| rs4690073 | AA | AG | GG | AG | GG | AA |
| rs363144 | TT | TT | TT | TT | TT | TT |
| rs3025838 | CC | CC | CC | CC | CC | CC |
| rs34315806 | TC | TC | CC | CC | CC | TT |
| rs363099 | TC | TC | CC | CC | CC | TT |
| rs363096 | CC | TC | TT | CC | TT | CC |
| rs2298967 | TC | TC | TT | TT | TT | CC |
| rs2298969 | GG | AG | AA | AG | AA | GG |
| rs6844859 | CC | TC | TT | TC | TT | CC |
| rs363092 | AA | AC | CC | AC | AA | AA |
| rs7685686 | GG | AG | AA | AG | AA | GG |
| rs363088 | TA | TA | AA | AA | AA | TT |
| rs362331 | CC | TC | TT | TC | TT | CC |
| rs916171 | GG | GC | CC | GC | CC | GG |
| rs362322 | AA | AA | AA | AA | AA | AA |
| rs362275 | TC | TC | CC | CC | CC | TT |
| rs362273 | AG | AG | AA | AA | AA | GG |
| rs2276881 | GG | GG | GG | GG | GG | GG |
| rs3121419 | TC | TC | CC | CC | CC | TT |
| rs362272 | — | AG | GG | GG | GG | AA |
| rs362271 | AG | AG | GG | GG | GG | AA |
| rs3775061 | AG | AG | AA | AA | AA | GG |
| rs362310 | TC | CC | CC | TC | CC | CC |
| rs362307 | CC | TC | CC | CC | CC | CC |
| rs362306 | AG | AG | GG | GG | GG | AA |
| rs362303 | TC | CC | CC | TC | CC | CC |
| rs362296 | AC | AC | AC | CC | CC | AA |

### Example 16: Allele-Specific Inhibition Measured in BacHD Cortical Neurons

Antisense oligonucleotides, ISIS 460209 (5′-TAAATTGTCATCACC-3′ (SEQ ID NO: 203)), targeting SNP rs7685686 of human HTT, and ISIS 387916 (TCTCTATTGCACATTCCAAG (SEQ ID NO: 6)), and with no human or murine SNP target site, were tested for their effect on Htt protein levels in vitro. ISIS 387916 is cross-reactive with murine Htt mRNA (GENBANK Accession No. NM_010414.1, designated herein as SEQ ID NO: 286) at target start site 5763 with one mismatch. ISIS 460209 is cross-reactive with murine Htt mRNA at target start site 6866 with three mismatches.

Primary BacHD cortical neurons, which express human Htt and murine Htt, were isolated in the following way: Embryos were dissected from E15.5-E17.5 pregnant females. Cortices were dissected into ice-cold divalent-free Hank's Balanced Salt Solution (Invitrogen, 14025-134). The cortices were chopped into pieces and digested with 0.05% Trypsin-EDTA (Invitrogen, 25300-120) at 37° C. for 8 minutes. The digestion was halted by addition of complete neurobasal media (Invitrogen, 10888-022). Cells were resuspended in media and treated with DNAse I (Invitrogen, 18047-019). After titration through a 100 ul pipette tip, cells are resuspended in neurobasal media with B27 supplement (Invitrogen, 17504-044), and counted. 1.7×10⁵ cells/well were plated in 24-well plates precoated with poly-D-lysine (BD Biosciences, 354210). Neurons were fed with 200 μl neurobasal media with B27 on the second day in vitro.

ISIS 460209 or ISIS 387916 was added to the supplementary media fed to neurons on division 2 at 0.7 μM, 1.4 μM or 1.5 μM final concentrations. Cells were harvested after 8 days with into 1 mL of media using a cell scraper. Cells were centrifuged at 2,500 rpm for 5 min at 4° C. and the pellets were resuspended in a buffer of 50 mM Tris, pH=8.0, 150 mM NaCl, 1% Igepal, 40 mM β-glycerophosphate, 10 mM NaF, 1× Roche complete protease inhibitor, 1 mM Sodium Orthovanadate and 800 μM PMSF. The lysates were centrifuged after 15 min incubation and protein concentration was measured with the DC assay (BioRad).

Protein lysates were run on low-bis gels to separate huntingtin alleles (resolving gel-2001: Acrylamide: BIS (10% acrylamide, 0.5% BIS, 375 mM Tris pH 8.8; stacking gel-4% Acrylamide-BIS (29:1), 156 mM Tris pH6.8; Running buffer-25 mM Tris, 190 mM Glycine, 0.1% SDS+10 μM beta-mercaptoethanol added fresh). After electrophoresis, proteins in the gel were transferred to a nitrocellulose membrane (Hybond-C Extra; GE Healthcare Bio-Sciences) at 90V for 40′ to allow samples to penetrate the stacking gel and then at 190V for 2.5 h to resolve proteins.

Primary antibodies specific for human Htt and murine calnexin protein were used at 1:10,000 dilutions. HRP-conjugated anti-mouse secondary antibody (1:10,000, Jackson ImmunoResearch Laboratories) was used for visualizing proteins using SuperSignal West Pico Chemiluminescent Substrate (Thermo Scientific). Protein bands were quantified using ImageJ software and normalized to calnexin levels. Protein bands were quantified using ImageJ software. Table 73 provides an estimate of the percentage inhibition relative to the negative control sample. The comparative percent inhibitions of the human Htt protein and the murine Htt protein are presented.

TABLE 73
Effect of antisense inhibition on mutant human and wild-type murine
Htt protein (percent inhibition normalized to PBS control)

| | Dose | | |
|---|---|---|---|
| | (μM) | Human | Murine |
| ISIS 387916 | 0.7 | 54 | 38 |
| | 1.4 | 75 | 58 |
| | 1.5 | 92 | 88 |
| ISIS 460209 | 0.2 | 71 | 35 |
| | 0.4 | 82 | 41 |
| | 1.5 | 94 | 56 |

### Example 17: Dose-Dependent Antisense Inhibition of Human Huntingtin mRNA Levels in Coriell Fibroblast Cell Lines

Gapmers from the studies described in Examples, 3, 4, 10, and 12 were selected and tested at various doses in GM04281, GM02171 and GM02173B cell lines. Each cell line was plated at a density of 25,000 cells per well and transfected using electroporation with 0.4747 nM, 1.5011 nM, 4.7463 nM, 15.0079 nM 45.455 nM, 150.0527 nM, 474.4673 nM, 1,500.27 nM, 4,743.833 nM, and 15,000 nM concentrations of antisense oligonucleotide, as specified in Tables 72, 73, and 74. After a treatment period of approximately 16 hours, RNA was isolated from the cells and HTT mRNA levels were measured by quantitative real-time PCR. Human HTT primer probe set RTS2617 was used to measure mRNA levels. HTT mRNA levels were adjusted according to total RNA content, as measured by RIBOGREEN. Results are presented as percent inhibition of HTT mRNA, relative to untreated control cells. IC₅₀ values are also provided in Tables 72, 73, and 74.

TABLE 74
Dose-dependent antisense inhibition of human HTT in GM04281 cells

| ISIS | 0.4747 | 1.5011 | 4.7463 | 15.0079 | 47.455 | 150.0527 | 474.4673 | 1500.27 | 4743.833 | 15000.0 | IC₅₀ |
|---|---|---|---|---|---|---|---|---|---|---|---|
| No | nM | nM | nM | nM | nM | nM | nM | nM | nM | nM | (μM) |
| 387916 | 15 | 12 | 4 | 5 | 7 | 26 | 70 | 89 | 98 | 99 | 0.33 |
| 435879 | 0 | 8 | 19 | 13 | 24 | 23 | 45 | 53 | 84 | 93 | 0.25 |
| 435890 | 16 | 1 | 8 | 12 | 25 | 23 | 32 | 52 | 61 | 91 | 0.82 |
| 460209 | 2 | 9 | 21 | 17 | 36 | 46 | 80 | 89 | 94 | 93 | 0.09 |
| 460210 | 4 | 7 | 5 | 19 | 20 | 35 | 69 | 85 | 98 | 98 | 0.21 |
| 476333 | 7 | 10 | 8 | 11 | 42 | 65 | 86 | 93 | 93 | 95 | 0.05 |

TABLE 75
Dose-dependent antisense inhibition of human HTT in GM02171 cells

| ISIS | 0.4747 | 1.5011 | 4.7463 | 15.0079 | 47.455 | 150.0527 | 474.4673 | 1500.27 | 4743.833 | 15000.0 | IC₅₀ |
|---|---|---|---|---|---|---|---|---|---|---|---|
| No | nM | nM | nM | nM | nM | nM | nM | nM | nM | nM | (μM) |
| 387916 | 22 | 8 | 0 | 9 | 0 | 32 | 60 | 90 | 96 | 97 | 0.27 |
| 435879 | 0 | 1 | 6 | 2 | 0 | 0 | 8 | 9 | 46 | 57 | 7.62 |
| 435890 | 0 | 0 | 0 | 6 | 0 | 0 | 0 | 31 | 27 | 71 | 4.37 |
| 460209 | 11 | 5 | 15 | 0 | 0 | 7 | 30 | 69 | 82 | 88 | 0.96 |
| 460210 | 0 | 0 | 0 | 2 | 17 | 18 | 38 | 70 | 93 | 95 | 0.56 |
| 476333 | 0 | 0 | 0 | 0 | 13 | 18 | 44 | 69 | 72 | 91 | 0.75 |

TABLE 76
Dose-dependent antisense inhibition of human HTT in GM02173B cells

| ISIS | 0.4747 | 1.5011 | 4.7463 | 15.0079 | 47.455 | 150.0527 | 474.4673 | 1500.27 | 4743.833 | 15000.0 | IC₅₀ |
|---|---|---|---|---|---|---|---|---|---|---|---|
| No | nM | nM | nM | nM | nM | nM | nM | nM | nM | nM | (μM) |
| 387916 | 3 | 17 | 7 | 25 | 27 | 33 | 65 | 88 | 98 | 99 | 0.19 |
| 435879 | 0 | 6 | 0 | 8 | 3 | 10 | 16 | 24 | 50 | 68 | 3.72 |
| 435890 | 0 | 13 | 0 | 1 | 2 | 12 | 16 | 23 | 49 | 82 | 4.60 |
| 460209 | 0 | 7 | 29 | 2 | 9 | 32 | 52 | 71 | 82 | 86 | 0.27 |
| 460210 | 0 | 13 | 0 | 5 | 16 | 18 | 49 | 74 | 93 | 97 | 0.27 |
| 476333 | 11 | 13 | 20 | 7 | 23 | 36 | 63 | 75 | 83 | 90 | 0.13 |

### Example 18: Validation of the Specificity of ISIS Oligonucleotides Targeting SNPs of Human Huntingtin by the Molecular Beacon Assay

Some of the gapmers from the study described in Example 17 were tested in GM04022 fibroblasts (from the Coriell Institute for Medical Research).

To verify allele-specific suppression of HTT mRNA in GM04022 fibroblasts by ISIS 435879, ISIS 460209, and ISIS 476333, the Molecular Beacon assay, as described in the van Bilsen at el publication (van Bilsen, P. H. J. et al., Human Gene Therapy. 19:710-718, 2008), was conducted using ‘molecular beacon’ synthetic oligonucleotides linked with a fluorophore and quencher. GM04022 fibroblasts were transfected by electroporation with ISIS 435879, ISIS 460209, or ISIS 476333 at 0.06 μM, 0.19 μM, 0.56 μM, 1.67 μM, 5 μM and 15 μM concentrations of antisense oligonucleotide, as specified in Tables 75-77. ISIS 387916 was included in the assay as a benchmark oligonucleotide. The qRT-PCR assay for molecular beacon for the A allele was conducted with the annealing temperature at 56.5° C. The qRT-PCR assay for molecular beacon for the C allele was conducted with the annealing temperature at 62.0° C. Primer probe set RTS2617 was used to measure the total HTT mRNA reduction. The results of the assay are presented in Tables 77-79 as percent inhibition over the PBS control. The results demonstrate that the SNP-specific ISIS oligonucleotides specifically target the C allele of rs7685686 compared to the A allele (Table 80).

TABLE 77
Dose-dependent antisense inhibition of the A
allele of rs7685686 in GM04022 fibroblasts

| ISIS | 0.06 | 0.19 | 0.56 | 1.67 | 5.00 | 15.00 | IC₅₀ |
|---|---|---|---|---|---|---|---|
| No | μM | μM | μM | μM | μM | μM | (μM) |
| 387916 | 33 | 40 | 53 | 90 | 99 | 98 | 0.56 |
| 435879 | 0 | 0 | 50 | 29 | 38 | 47 | 10.8 |
| 460209 | 14 | 4 | 54 | 73 | 81 | 95 | 0.53 |
| 476333 | 2 | 44 | 41 | 77 | 91 | 86 | 0.64 |

TABLE 78
Dose-dependent antisense inhibition of the C
allele of rs7685686 in GM04022 fibroblasts

| ISIS | 0.06 | 0.19 | 0.56 | 1.67 | 5.00 | 15.00 | IC₅₀ |
|---|---|---|---|---|---|---|---|
| No | μM | μM | μM | μM | μM | μM | (μM) |
| 387916 | 41 | 42 | 46 | 86 | 95 | 92 | 0.54 |
| 435879 | 0 | 0 | 75 | 60 | 68 | 81 | 2.9 |
| 460209 | 35 | 48 | 76 | 84 | 88 | 92 | 0.19 |
| 476333 | 22 | 60 | 75 | 84 | 90 | 93 | 0.15 |

TABLE 79
Dose-dependent antisense inhibition of
total HTT mRNA in GM04022 fibroblasts

| ISIS | 0.06 | 0.19 | 0.56 | 1.67 | 5.00 | 15.00 |
|---|---|---|---|---|---|---|
| No | μM | μM | μM | μM | μM | μM |
| 387916 | 32 | 59 | 49 | 89 | 98 | 99 |
| 435879 | 0 | 0 | 42 | 25 | 41 | 62 |
| 460209 | 26 | 27 | 54 | 75 | 84 | 96 |
| 476333 | 25 | 51 | 58 | 82 | 92 | 90 |

TABLE 80
IC₅₀ ratio (A/C) in GM04022 fibroblasts

| ISIS No | Ratio |
|---|---|
| 387916 | 1.0 |
| 435879 | 4.2 |
| 460209 | 2.8 |
| 476333 | 4.3 |

### Example 19: Allele-Specific Inhibition Measured in Cortical Neurons from BACHD and YAC18 Mice

In order to identify potential SNPs for screening of human allele-specific ISIS oligonucleotides, the HTT mRNA of YAC18 and BACHD mice were sequenced by the Goldengate 96SNP assay. It was determined that the BAC and YAC mice carried different alleles at several key SNP positions (Table 72) and could therefore be used as a screening tool for allele-specific knockdown. Each of the SNP positions chosen for targeting in the mouse strains were also compared to human HD chromosomes. For each target, approximately 50% of the human HD population is heterozygous for the target expressed in the BACHD mice, but not the YAC18 mice.

In order to verify the allele-specificity of the ISIS oligonucleotides (described in Examples 2, 9, 17 and 18), the antisense oligonucleotides, ISIS 460207, targeting SNP rs362331; ISIS 460209, targeting SNP rs7685686; ISIS 435879, targeting SNP rs7685686; ISIS 476333, targeting SNP rs7685686; ISIS 460210, targeting SNP rs2298969; ISIS 435874, targeting SNP rs4690072; ISIS 460208, targeting SNP rs4690072; ISIS 435331, targeting SNP rs2024115; and ISIS 435871, targeting SNP rs363088, were tested for their effect on HTT protein levels in BACHD and YAC18 cortical neurons. ISIS 387916, which has no human or murine SNP target site, was used as the benchmark. ISIS 387916 is cross-reactive with murine HTT mRNA (GENBANK Accession No. NM_010414.1, designated herein as SEQ ID NO: 286) at target start site 5763 with one mismatch. It was expected that treatment with the allele-specific antisense oligonucleotides would cause significant inhibition of HTT mRNA in the BACHD neurons and not in the YAC18 neurons. It was also expected that treatment with ISIS 387916 would cause inhibition of HTT mRNA in both sets of neurons.

YAC18 cultures were prepared from E16.5 pregnant female YAC18 (line 60, +/+) mice who had been bred with YAC18 (line 60, +/+) males. All progeny are thus homozygous YAC18 (line 60), facilitating pooled cortical cultures. BACHD E16.5 embryos were isolated from pregnant BACHD (+/−) mice who had been bred with pregnant BACHD (+/−) male mice, necessitating single pup cultures and genotyping. Single cortices were isolated, using caution to prevent cross-contamination of samples. Each dissociated cortex was used to seed 5 wells of a 6-well plate. After genotyping, only BACHD (+/−) cultures were used for ASO treatment. The antisense oligonucleotides were added to the supplementary media fed to the neurons on division 2. Cells were harvested after 8 days with into 1 mL of media using a cell scraper. Cells were centrifuged at 2,500 rpm for 5 min at 4° C. and the pellets were resuspended in a buffer of 50 mM Tris, pH=8.0, 150 mM NaCl, 1% Igepal, 40 mM β-glycerophosphate, 10 mM NaF, 1× Roche complete protease inhibitor, 1 mM Sodium Orthovanadate and 800 μM PMSF. The lysates were centrifuged after 15 min incubation and protein concentration was measured with the DC assay (BioRad).

Protein lysates were run on low-bis gels to separate huntingtin alleles (resolving gel-2001: Acrylamide: BIS (10% acrylamide, 0.5% BIS, 375 mM Tris pH 8.8; stacking gel-4% Acrylamide-BIS (29:1), 156 mM Tris pH6.8; Running buffer-25 mM Tris, 190 mM Glycine, 0.1% SDS+10 μM beta-mercaptoethanol added fresh). After electrophoresis, proteins in the gel were transferred to a nitrocellulose membrane (Hybond-C Extra; GE Healthcare Bio-Sciences) at 90V for 40′ to allow samples to penetrate the stacking gel and then at 190V for 2.5 h to resolve proteins.

Primary antibodies specific for human HTT and murine calnexin protein were used at 1:10,000 dilutions. HRP-conjugated anti-mouse secondary antibody (1:10,000, Jackson ImmunoResearch Laboratories) was used for visualizing proteins using SuperSignal West Pico Chemiluminescent Substrate (Thermo Scientific). Protein bands were quantified using ImageJ software and normalized to calnexin levels. Tables 81-91 provide the percentage inhibition relative to the untreated control sample. The percentage inhibition of human HTT protein levels in BACHD and YAC18 neurons are presented.

TABLE 81
HTT SNPs in BACHD and YAC18 mice and
correlation with human HTT SNPs

| | Allele | Allele | Allele present | % of human |
|---|---|---|---|---|
| | present in | present in | in human patients | patients |
| | YAC18 | BACHD | with high | heterozgous at |
| SNP | Mice | Mice | CAG repeats | the SNP position |
| rs2024115 | G | A | A | 48 |
| rs2298969 | G | A | A | 52 |
| rs362331 | C | T | T | 49 |
| rs363088 | G | T | T | 38 |
| rs4690072 | T | A | A | 49 |
| rs7685686 | G | A | A | 49 |

TABLE 82
Effect of antisense inhibition by ISIS
387916 in BACHD and YAC18 neurons

| | 500 nM | 1500 nM |
|---|---|---|
| YAC18 | 69 | 81 |
| BACHD | 84 | 90 |

TABLE 83
Effect of antisense inhibition by ISIS 435331,
targeting rs2024115 in BACHD and YAC18 neurons

| | 500 nM | 1500 nM |
|---|---|---|
| YAC18 | 0 | 0 |
| BACHD | 39 | 43 |

TABLE 84
Effect of antisense inhibition by ISIS 460210,
targeting rs2298969 in BACHD and YAC18 neurons

| | 500 nM | 1500 nM |
|---|---|---|
| YAC18 | 31 | 51 |
| BACHD | 79 | 89 |

TABLE 85
Effect of antisense inhibition by ISIS 460207,
targeting rs362331 in BACHD and YAC18 neurons

| | 500 nM | 1500 nM |
|---|---|---|
| YAC18 | 0 | 0 |
| BACHD | 29 | 44 |

TABLE 86
Effect of antisense inhibition by ISIS 435871,
targeting rs363088 in BACHD and YAC18 neurons

| | 500 nM | 1500 nM |
|---|---|---|
| YAC18 | 0 | 0 |
| BACHD | 51 | 68 |

TABLE 87
Effect of antisense inhibition by ISIS 435874,
targeting rs4690072 in BACHD and YAC18 neurons

| | 500 nM | 1500 nM |
|---|---|---|
| YAC18 | 9 | 5 |
| BACHD | 30 | 44 |

TABLE 88
Effect of antisense inhibition by ISIS 460208,
targeting rs4690072 in BACHD and YAC18 neurons

| | 500 nM | 1500 nM |
|---|---|---|
| YAC18 | 1 | 8 |
| BACHD | 54 | 68 |

TABLE 89
Effect of antisense inhibition by ISIS 460209,
targeting rs7685686 in BACHD and YAC18 neurons

| | 500 nM | 1500 nM |
|---|---|---|
| YAC18 | 12 | 32 |
| BACHD | 72 | 83 |

TABLE 90
Effect of antisense inhibition by ISIS 435879,
targeting rs7685686 in BACHD and YAC18 neurons

| | 500 nM | 1500 nM |
|---|---|---|
| YAC18 | 0 | 7 |
| BACHD | 36 | 58 |

TABLE 91
Effect of antisense inhibition by ISIS 476333,
targeting rs7685686 in BACHD and YAC18 neurons

| | 500 nM | 1500 nM |
|---|---|---|
| YAC18 | 46 | 61 |
| BACHD | 89 | 91 |

1.-102. (canceled)

103. A compound comprising a modified oligonucleotide consisting of 15 to 20 linked nucleosides and having at least 90% sequence identity to the nucleobase sequence of SEQ ID NO: 8, 9, 10, 11, or 240, wherein the modified oligonucleotide comprises at least one nucleoside comprising a modified sugar and at least one modified internucleoside linkage, and wherein each T in SEQ ID NO: 8, 9, 10, 11, or 240 is independently a T or a U.

104. The compound of claim 103, wherein the modified oligonucleotide has at least 95% sequence identity to the nucleobase sequence of SEQ ID NO: 8, 9, 10, 11, or 240.

105. The compound of claim 103, wherein the modified oligonucleotide has 100% sequence identity to the nucleobase sequence of SEQ ID NO: 8, 9, 10, 11, or 240.

106. The compound of claim 103, wherein the modified oligonucleotide consists of 20 linked nucleosides.

107. The compound of claim 103, wherein the modified sugar is a 2′MOE sugar.

108. The compound of claim 103, wherein the modified sugar is a 2′-OMe sugar.

109. The compound of claim 103, wherein the at least one modified internucleoside linkage is a phosphorothioate internucleoside linkage.

110. The compound of claim 103, wherein at least one nucleoside comprises a modified nucleobase.

111. The compound of claim 110, wherein the modified nucleobase is a 5-methylcytosine.

112. The compound of claim 103, wherein the modified oligonucleotide is a gapmer.

113. The compound of claim 113, wherein the gapmer has a 5-10-5 wing-gap-wing motif.

114. A pharmaceutical composition comprising the compound of claim 103 and a pharmaceutically acceptable diluent or carrier.

115. A compound comprising a modified oligonucleotide consisting of 15 to 20 linked nucleosides, wherein the modified oligonucleotide is complementary to a differentiating polymorphism site, wherein the modified oligonucleotide comprises at least a 15 contiguous nucleobase portion of the nucleobase sequence of any of SEQ ID NOs: 8-11 and 240, wherein position 8, 9, or 10 of the modified oligonucleotide, as counted from the 5′ terminus of the modified oligonucleotide, aligns with the differentiating polymorphism site, wherein the modified oligonucleotide comprises at least one nucleoside comprising a modified sugar and at least one modified internucleoside linkage, and wherein each T in SEQ ID NO: 8, 9, 10, 11, or 240 is independently a T or a U.

116. The compound of claim 115, wherein the modified oligonucleotide comprises a 16, 17, 18, or 19 contiguous nucleobase portion of the nucleobase sequence of any of SEQ ID NOs: 8-11 and 240.

117. The compound of claim 115, wherein the modified oligonucleotide consists of 20 linked nucleosides.

118. The compound of claim 115, wherein the modified sugar is a 2′MOE sugar.

119. The compound of claim 115, wherein the modified sugar is a 2′-OMe sugar.

120. The compound of claim 115, wherein the at least one modified internucleoside linkage is a phosphorothioate internucleoside linkage.

121. The compound of claim 115, wherein at least one nucleoside comprises a modified nucleobase.

122. The compound of claim 121, wherein the modified nucleobase is a 5-methylcytosine.

123. The compound of claim 115, wherein the modified oligonucleotide is a gapmer.

124. The compound of claim 123, wherein the gapmer has a 5-10-5 wing-gap-wing motif.

125. A pharmaceutical composition comprising the compound of claim 115 and a pharmaceutically acceptable diluent or carrier.

126. A compound comprising a modified oligonucleotide consisting of 15 to 20 linked nucleosides and having a nucleobase sequence comprising a portion that is complementary to at least 15 contiguous nucleobases of nucleobases 19815 to 19835 of SEQ ID NO: 1, wherein the modified oligonucleotide comprises at least one nucleoside comprising a modified sugar and at least one modified internucleoside linkage.

127. The compound of claim 126, wherein the modified oligonucleotide has a nucleobase sequence comprising a portion that is complementary to at least 16, 17, 18, or 19 contiguous nucleobases of nucleobases 19815 to 19835 of SEQ ID NO: 1.

128. The compound of claim 126, wherein the modified oligonucleotide consists of 20 linked nucleosides.

129. The compound of claim 126, wherein the modified sugar is a 2′MOE sugar.

130. The compound of claim 126, wherein the modified sugar is a 2′-OMe sugar.

131. The compound of claim 126, wherein the at least one modified internucleoside linkage is a phosphorothioate internucleoside linkage.

132. The compound of claim 126, wherein at least one nucleoside comprises a modified nucleobase.

133. The compound of claim 132, wherein the modified nucleobase is a 5-methylcytosine.

134. The compound of claim 126, wherein the modified oligonucleotide is a gapmer.

135. The compound of claim 134, wherein the gapmer has a 5-10-5 wing-gap-wing motif.

136. A pharmaceutical composition comprising the compound of claim 126 and a pharmaceutically acceptable diluent or carrier.
